(19) United States
(12) Patent Application Publication
(10) Pub. No.: US 2007/0128183 A1
(43) Pub. Date: Jun. 7, 2007

(54) Td antigens

(75) Inventors: Andreas Meinke, Pressbaum (AT); Christine Triska, Vienna (AT); Tamas Henics, Vienna (AT); Duc Minh Bui, Vienna (AT); Eszter Nagy, Vienna (AT); Sonja Prustomersky, Vienna (AT)

Correspondence Address: FULBRIGHT & JAWORSKI L.L.P., 600 CONGRESS AVE., SUITE 2400, AUSTIN, TX, 78701, US

(73) Assignee: INTERCELL AG, Vienna (AT)

(21) Appl. No.: 11/568,436

(22) PCT Filed: Apr. 26, 2005
(86) PCT No.: PCT/EP05/51857

§ 371 Date: Oct. 27, 2006

(30) Foreign Application Priority Data
Apr. 27, 2004 (EP) 04450095.7

(51) Int. Cl. A61K 39/395; C12P 21/04

(52) U.S. Cl. 424/130.1; 435/70.21

(57) ABSTRACT

The present invention discloses isolated nucleic acid molecules encoding a hyperimmune serum reactive antigen or a fragment thereof as well as hyperimmune serum reactive antigens or fragments thereof from enteroaggregative E. coli, enterotexigenic E. coli, S. flexneri and C. jejuni, methods for isolating such antigens and specific uses thereof.

The present invention relates to isolated nucleic acid molecules, which encode antigens for bacterial pathogens causing travelers' diarrhea, which are suitable for use in preparation of pharmaceutical medicaments for the prevention and treatment of bacterial infections caused by Escherichia coli, Shigella flexneri and Campylobacter jejuni.

Travelers' diarrhea (TD) is a syndrome characterized by a twofold or greater increase in the frequency of unformed bowel movements and is the most frequent health-problem affecting travelers from industrialised to high-risk countries. Commonly associated symptoms include abdominal cramps, nausea, bloating, urgency, fever, and malaise. Episodes of TD usually begin abruptly, occur during travel or soon after returning home, and are generally self-limited. The most important determinant of risk is the destination of the traveler, but also point of origin, host factors and exposure to contaminated food or water are main risk factors. Attack rates of 20% to 50% are commonly reported among the approximately 50 million travelers from industrialized to developing countries each year. High-risk destinations include most of the developing countries of Latin America, Africa, the Middle East, and Asia. Intermediate-risk destinations include most of the southern European countries and a few Caribbean islands. Low-risk destinations include Canada, northern Europe, Australia, New Zealand, the United States, and some of the Caribbean islands.

TD is slightly more common in young adults than in older people. The reasons for this difference are unclear, but could include a lack of acquired immunity, more adventurous travel styles, and different eating habits. Attack rates are similar in men and women. The onset of TD is usually within the first week of travel, but can occur at any time during the visit and even after returning home.

TD is acquired through ingestion of fecally contaminated food or water, or both. Both cooked and uncooked foods might be implicated if they have been improperly handled. Especially risky foods include raw or undercooked meat and seafood and raw fruits and vegetables. Tap water, ice, and unpasteurized milk and dairy products can be associated with increased risk of TD.

Infectious agents are the primary cause of TD. Travelers from developed countries to developing countries frequently experience a rapid, dramatic change in the type of organisms in their gastrointestinal tract, and often, potential enteric pathogens are among these new organisms. Those individuals, who develop diarrheal disease, have ingested an inoculum of virulent organisms sufficiently large to overcome individual defense mechanisms, resulting in symptoms. The bacterial causative agents for travelers' diarrhea are listed below. They constitute approximately 80% of all identified cases of TD.

| Pathogen | Epidemiological feature |
|---|---|
| Enterotoxigenic and | Worldwide, summer |
| enteroaggregative E. coli (ETEC, | |
| EAEC) | |
| Invasive E. coli | Uncommon cause, wet season |
| Hemorrhagic E. coli | Uncommon cause, food-borne |
| Shigella spp. | Worldwide, summer |
| Campylobacter jejuni, other spp. | Worldwide, year round, esp. winter, |
| | animals |
| Aeromonas (4 species) | Worldwide, summer |
| Plesiomonas shigelloides | Worldwide, summer, Japan |
| Nontyphoidal Salmonella | Worldwide, summer |
| Typhoidal Salmonella | Worldwide, summer |
| Vibrio parahemolyticus | Coastal areas, summer, seafood |
| Yersinia enterocolitica | Northern climatzes, year round, esp. |
| | winter, meats, dogs |
| Vibrio cholera | Indian subcontinent, South Africa |
| Vibrio fluvialis | |

Travelers' Diarrhea

TD typically results in three to five loose or watery stools per day. The median duration of untreated diarrhea is 3 to 5 days. Approximately 10% of the cases persist longer than 1 week, approximately 2% longer than 1 month, and <1% longer than 3 months. Persistent diarrhea is, thus, quite uncommon and can differ considerably from acute TD with respect to etiology and risk factors. Diarrhea is accompanied by abdominal cramps in 50-73%, malaise in 50-58%, nausea in 46-50%, feverish feeling in 37%, bloody dysentery in 2-10%, and vomiting in 8-15%. More than half of the cases are mild and do not confine the travelers' activities, but 20% are severe and will confine the traveler to the hotel room for 2-3 days. Travelers can experience more than one episode of TD during a single trip. Rarely is TD life threatening. Diarrhea results from either an imbalance in the absorptive-secretory processes of the bowel or from intestinal hypermotility. Usually, the intestinal tract is a site of absorption for water and electrolytes. In the case of diarrhea, this physiological process is reversed, and the gut becomes the site of water and electrolyte loss. Most of the morbidity from diarrheal illness is due to dehydration and electrolyte imbalances.

The infectious dose for microorganisms varies from 10¹ to 10² organisms for Shigella and Giardia lamblia up to 10⁸ for Vibrio cholerae and Escherichia coli. Factors that predispose one to acquiring diarrheal illness include H₂-blocker use, broad-spectrum antibiotic use, abnormal intestinal motility, and interruption of the gastric mucosa. Diarrheal disease is caused by pathogens that are typically categorized as inflammatory and non-inflammatory. Inflammatory pathogens (e.g., Shigella) invade the bowel mucosa. Non-inflammatory or “secretory” pathogens (e.g. E. coli) elaborate enterotoxins. Some bacteria that cause “food poisoning” elaborate toxins even prior to ingestion (e.g. Staphylococcus aureus, Bacillus cereus). Enterotoxin-induced diarrheal stool is watery and contains few fecal leukocytes. The prototypic form of secretory diarrhea (cholera) is caused by V. cholerae. In cholera, large amounts of isotonic solution are passed into the bowel, making the patient prone to dehydration. A similar pattern of diarrhea is seen with viruses and enterotoxic E. coli. Inflammatory bacteria enter the endothelium of the distal small bowel and colon, producing fever and abdominal pain. The stool may contain blood, mucus, and abundant fecal leukocytes. Common invasive organisms are Shigella, Campylobacter jejuni, and Salmonella. Yersinia enterocolitica and some forms of E. coli are also invasive.

Diarrhea in Children

Infants, 2 years of age or younger are at high risk of acquiring TD. The greatest risk to the infant with diarrhea is dehydration. It is recommended by the WHO to give Oral Rehydration Solution (ORS) for Diarrheal Illness to children to prevent dehydration. Nevertheless, diarrhea is still one of the major causes (10 to 20% of deaths to children under age of 5) contributing to the high mortality rate for children in developing countries (40 to 60 deaths/1,000 live birth).

In the United States, despite the many improvements in water treatment, sanitation, education, and medical care, diarrhea remains one of the most common pediatric illnesses. Each year, children less than 5 years of age experience 20-35 million episodes of diarrhea, which result in 2-3.5 million doctor visits, greater than 200,000 hospitalizations, and 325-425 deaths. Approximately 65% of the hospitalizations and 85% of the diarrheal deaths occur in the first year of life. While Rotavirus is the most common cause of acute diarrhea among children, accounting for one-fourth of all cases {Cohen, M., 1991}, many other viruses can cause childhood diarrhea as well, including Norwalk-like viruses, enteric adenoviruses, astroviruses, and caliciviruses. Importantly, bacterial pathogens including Shigella, Campylobacter, Salmonella and certain strains of Escherichia coli, are also causes of acute diarrhea in children. Most of these bacterial pathogens are subject to the present invention.

Foodborne Diseases

The most common foodborne diseases are infections caused by such bacteria as Salmonella and Campylobacter, or by viruses, such as Norwalk or hepatitis A. Food poisoning caused by bacteria comprises about two-thirds of U.S. poisoning outbreaks linked to a known etiology. C. jejuni alone was responsible for more than 2 million illnesses in the United States in 1999. Specifically, about 75% of reported cases of bacterial food poisoning in the United States can be traced to Campylobacter, Salmonella, Staphylococcus and Clostridium perfringens. In addition, the two bacterial pathogens Shigella and ETEC are also listed among those causing food-borne diarrheal diseases.

Travelers' diarrhea caused by bacterial pathogens in travelers from developed countries accounts for at least 10 million cases of disease per year. Despite high sanitation standards in developed countries, there are for example in the U.S.A. still millions of cases reported every year, especially among children below 5 years of age, leading to millions of pediatric doctor office visits and a large burden on the healthcare system of the respective countries. In addition, diarrhea caused by enteric bacterial pathogens still kills many children in developing countries and is responsible also for the death of hundreds of children per year in developed countries.

At present, there is no vaccine on the market that could prevent diarrheal diseases and treatment relies especially in more severe cases mainly on antibiotics. Travelers who develop diarrhea with three or more loose stools in an 8-hour period, especially if associated with nausea, vomiting, abdominal cramps, fever, or blood in the stools, might benefit from antimicrobial treatment. A typical 3- to 5-day illness can often be shortened to 1 to 1½ days by effective antimicrobial agents. For these more severe forms of travelers' diarrhea an antimicrobial treatment may be effective, but is dependent on the etiologic agent and its antibiotic sensitivity. The antibiotic regimen most likely to be effective is ciprofloxacin (500 mg) taken twice a day. Other fluroquinolones, such as norfloxacin, ofloxacin or levofloxacin might be equally as effective. Fewer side effects and less widespread antibiotic resistance have been reported with the fluoroquinolones than with TMP/SMX. Three days of treatment is recommended, although 2 or fewer days might be sufficient. Bismuth subsalicylate (Pepto-Bismol) also may be used as treatment: 1 fluid ounce or two 262 mg tablets every 30 minutes for up to eight doses in a 24 hour period, which can be repeated on a second day. At present there is no report on marketed vaccines for the four pathogens subject of this patent.

Besides travelers' diarrhea, the same enteric bacterial pathogens cause millions of deaths yearly among young children in developing countries. Furthermore, the enteric bacterial pathogens are very frequently the source of food-borne diseases (see above). Estimates of the number of cases of food-borne disease in the United States range from 6.5 to 81 million cases per year, with from 525 to more than 7000 associated deaths. Thus, there remains a need for an effective treatment to prevent bacterial infections causing diarrhea and travelers' diarrhea. A vaccine could not only prevent relative mild diarrheal diseases, but also many cases of severe disease and high mortality rates in developing countries caused by the targeted pathogens. In developed countries, a vaccine could furthermore prevent the recently increasing cases of food borne diseases caused especially by C. jejuni and E. coli.

The importance of surface proteins in human immunity to enteric pathogens already has been appreciated. It is apparent that all pathogens express surface proteins with activity relevant to host immune defense; Fimbriae proteins in pathogenic E. coli (e.g. CS2 and CS3; {Altboum, Z. et al., 2001}) and invasion proteins from Shigella spp. {Turbyfill, K. et al., 2000} for example have been shown to be immunogenic and protective in animal models of the respective disease. The major problem with some of these proteins as vaccine candidates seems to be their variability in prevalenve among the different clinical isolates of the enteric pathogens. Thus there is a need to systematically identify vaccine candidates conserved in various clinically relevant strains in order to provide protection against human diarrheal disease as caused by the heterogeneity of clinical strains of the various pathogens.

A vaccine can contain a whole variety of different antigens. Examples of antigens are whole-killed or attenuated organisms, subfractions of these organisms/tissues, proteins, or, in their most simple form, peptides. Antigens can also be recognized by the immune system in form of glycosylated proteins or peptides and may also be or contain polysaccharides or lipids. Short peptides can be used since for example cytotoxic T-cells (CTL) recognize antigens in form of short usually 8-11 amino acids long peptides in conjunction with major histocompatibility complex (MHC). B-cells can recognize linear epitopes as short as 4-5 amino acids, as well as three-dimensional structures (conformational epitopes). In order to obtain sustained, antigen-specific immune responses, adjuvants need to trigger immune cascades that involve all cells of the immune system. Primarily, adjuvants are acting, but are not restricted in their mode of action, on so-called antigen presenting cells (APCs). These cells usually first encounter the antigen(s) followed by presentation of processed or unmodified antigen to immune effector cells. Intermediate cell types may also be involved. Only effector cells with the appropriate specificity are activated in a productive immune response. The adjuvant may also locally retain antigens and co-injected other factors. In addition the adjuvant may act as a chemoattractant for other immune cells or may act locally and/or systemically as a stimulating agent for the immune system.

Vaccine development for enteric bacterial diseases has focused on several approaches including attenuated strains, O polysaccharide-based conjugates and proteosomes, but a safe, effective product is still not available. However, ETEC and Shigella are ranked as important targets by the World Health Organisation for the development of vaccines. Currently a number of vaccines against infection by enteric bacterial pathogens are in the research stages of development Most of these efforts are focused on strategies using attenuated, live or killed whole cell vaccine preparations. Only recently were approaches introduced based on recombinant proteins. However, the present invention for the first time assesses the possibility to design a polypeptide-based vaccine against more than one bacterial enteric pathogen

Protein vaccines are without any doubt of great value for the prevention of TD disease, because the vaccine can cover a broad range of bacterial serotypes and is therefore much less prone to ecological pressure, leading to a replacement disease by novel serotypes, in comparison to polysaccharide-based vaccines. This already suggests that there is a need to develop new generation vaccines composed of proteins, or their derivatives, expressed by all strains under in vivo conditions with the ability to induce opsonizing and/or neutralizing antibodies in humans.

Certain proteins or enzymes displayed on the surface of or secreted by gram-negative enteric pathogens significantly contribute to pathogenesis and are involved in the disease process caused by these pathogens. Often, these proteins are involved in direct interactions with host tissues or constitute toxins responsible for cytotoxic effects on mucosal host cells. Several surface proteins are characterized as virulence factors, important for pathogenicity, such as the fimbriae proteins of ETEC and EAEC, the flagellin proteins of C. jejuni or the invasin proteins of Shigella. The use of some of the above-described proteins as antigens for potential vaccines as well as a number of additional candidates resulted mainly from a selection based on easiness of identification or chance of availability. There is a demand to identify relevant antigens for enteric bacterial pathogens, which may be shared among several bacteria, in a more comprehensive way.

The present inventors have developed a method for identification, isolation and production of hyperimmune serum reactive antigens from a specific pathogen, especially from Staphylococcus aureus and Staphylococcus epidermidis (WO 02/059148). However, given the differences in biological property, pathogenic function and genetic background, the Gram-negative enteric pathogens described in this patent are very distinct from the Gram-positive Staphylococcus strains. Importantly, the selection of sera for the identification of antigens from the enteric pathogens is different from that applied to the S. aureus screens. Serum samples to be used as antibody sources were collected from healthy adults living in endemic areas, such as Bangladesh and Egypt, since these individuals encounter the diarrhogenic pathogens multiple times in their life and they become protected by developing pathogen specific antibodies.

To be able to select for relevant serum sources, a series of ELISA and immunoblotting experiments measuring pathogen-specific IgG antibody levels were performed with bacterial wole cells, lysates and culture supernatant proteins. Sera determined to have the highest titer against the individual pathogens were pooled (5 samples/pool) and IgG purified for screening purposes.

The present invention applies a high throughput genomic method to identify in vivo expressed pathogen-specific proteins with the ability to induce antibodies in humans during the course of infections and colonization.

The genomes of the Gram-negative enteric bacteria ETEC, EAEC and S. flexneri are closely related, whereas the genome of C. jejuni is only distantly related to the former three bacterial genomes. Importantly, all four genomes show a number of important differences as compared to the genome of S. aureus. The genomes of ETEC, EAEC and S. flexneri contain app. 4.6 Mb, while S. aureus harbours 2.85 Mb and C. jejuni only 1.64 Mb. While E. coli and Shigella genomes have an average GC content of more than 50%, S. aureus only contains 33%, and C. jejuni app. 30% GC bases. Approximately 52% of the encoded genes of the S. aureus genome are shared with the E. coli and Shigella genomes, while less than 40% are shared between S. aureus and C. jejuni. In addition, S. aureus requires different growth conditions and media for propagation than the enteric pathogens. A list of the most important diseases, which can be inflicted by S. aureus and enteric pathogens is presented below. S. aureus causes mainly nosocomial, opportunistic infections: impetigo, folliculitis, abscesses, boils, infected lacerations, endocarditis, meningitis, septic arthritis, pneumonia, osteomyelitis, scalded skin syndrome (SSS), toxic shock syndrome. The enteric pathogens cause mainly mild to life-threatening diarrheal disease. C. jejuni infections may also lead to a disease termed Guillain-Barré syndrome.

The complete genome sequence has been determined for C. jejuni NCTC11168, two S. flexneri serotype 2a strains (301 and 2457T), but not for the two E. coli strains ETEC and EAEC. Genomic sequences are available for the E. coli strains K12 and enteropathogenic O157:H7 (see http://www.ncbi.nlm.nih.gov/genomes/Complete.html or http://www.tigr.org/tdb/mdb/mdbcomplete.html).

The problem underlying the present invention was to provide means for the development of medicaments such as vaccines against bacterial pathogens causing diarrheal disease. More particularly, the problem was to provide an efficient, relevant and comprehensive set of nucleic acid molecules or hyperimmune serum reactive antigens from enteroaggregative and enterotoxigenic E. coli, S. flexneri and C. jejuni that can be used for the manufacture of said medicaments.

Therefore, the present invention provides an isolated nucleic acid molecule encoding a hyperimmune serum reactive antigen or a fragment thereof comprising a nucleic acid sequence, which is selected from the group consisting of: a nucleic acid molecule having at least 70% sequence identity to a nucleic acid molecule selected from Seq ID No 2-4, 7-9, 14-17, 19, 23-24, 26, 29-30, 33-35, 39-40, 42, 48-49, 52-53, 57-58, 60-64, 66-74, 76-79, 82-84, 86-91, 93-95, 97-99, 103-104, 109-110, 114-118, 121-123, 126-127, 132, 138-142, 145-146, 149, 151-152, 154-161, 163, 165, 167-168, 170-172, 174-300, 925-926, 928-935, 937-946, 949-953, 955-961, 964, 966-970, 972-996, 1069-1108, 1110-1202, 602, 605-607, 612-614, 616, 620-621, 623-624, 626, 628-629, 633-634, 637-638, 641-644, 646-656, 659-664, 666-667, 669-671, 675, 678-679, 681-683, 685-686, 689-690, 695, 698-701, 704-705, 707-762 and 1337-1364.
a) a nucleic acid molecule which is complementary to the nucleic acid molecule of a),
b) a nucleic acid molecule comprising at least 15 sequential bases of the nucleic acid molecule of a) or b)
c) a nucleic acid molecule which anneals under stringent hybridisation conditions to the nucleic add molecule of a), b), or c)
d) a nucleic acid molecule which, but for the degeneracy of the genetic code, would hybridise to the nucleic add molecule defined in a), b), c) or d).

According to a preferred embodiment of the present invention the sequence identity is at least 80%, preferably at least 95%/,, especially 100%.

Furthermore, the present invention provides an isolated nucleic acid molecule encoding a hyperimmune serum reactive antigen or a fragment thereof comprising a nucleic acid sequence selected from the group consisting of
a) a nucleic acid molecule having at least 96%, preferably at least 98%, especially 100 % sequence identity to a nucleic acid molecule selected from Seq ID No 10-13, 20-21, 27-28, 31-32, 41, 44-47, 50-51, 54-56, 59, 80, 85, 96, 100, 105-107, 112, 124-125, 128-131, 137, 143, 162, 166, 169, 936, 948, 954, 963, 971, 608-611, 617-618, 622, 631-632, 635-636, 639-640, 657, 668, 672, 676-677, 680, 687-688, 691-694 and 702.
b) a nucleic acid molecule which is complementary to the nucleic acid molecule of a),
c) a nucleic acid molecule comprising at least 15 sequential bases of the nucleic acid molecule of a) or b)
d) a nucleic acid molecule which anneals under stringent hybridisation conditions to the nucleic add molecule of a), b) or c),
e) a nucleic acid molecule which, but for the degeneracy of the genetic code, would hybridise to the nucleic acid defined in a), b), c) or d).

According to another aspect, the present invention provides an isolated nucleic acid molecule comprising a nucleic acid sequence selected from the group consisting of
a) a nucleic acid molecule selected from Seq ID No 1, 5-6, 18, 22, 25, 36, 38, 43, 65, 75, 81, 92, 101-102, 108, 111, 113, 119-120, 133-136, 144, 147-148, 150, 153, 164, 173, 927, 947, 962, 965, 1109, 601, 603-604, 615, 619, 625, 627, 630, 645, 658, 665, 673-674, 684, 696-697, 703 and 706.
b) a nucleic acid molecule which is complementary to the nucleic acid of a),
c) a nucleic acid molecule which, but for the degeneracy of the genetic code, would hybridise to the nucleic acid defined in a), b), c) or d).

Preferably, the nucleic acid molecule is DNA or RNA.

According to a preferred embodiment of the present invention, the nucleic acid molecule is isolated from a genomic DNA, especially from a enteroaggregative E. coli, enterotoxigenic E. coli, S. flexneri and C. jejuni genomic DNA.

According to the present invention a vector comprising a nucleic acid molecule according to any of the present invention is provided.

In a preferred embodiment the vector is adapted for recombinant expression of the hyperimmune serum reactive antigens or fragments thereof encoded by the nucleic acid molecule according to the present invention.

The present invention also provides a host cell comprising the vector according to the present invention.

According to another aspect the present invention further provides a hyperimmune serum-reactive antigen comprising an amino acid sequence being encoded by a nucleic acid molecule according to the present invention.

In a preferred embodiment the amino add sequence (polypeptide) is selected from the group consisting of Seq ID No 302-304, 307-309, 314-317, 319, 323-324, 326, 329-330, 333-335, 339-340, 342, 348-349, 352-353, 357-358, 360-364, 366-374, 376-379, 382-384, 386-391, 393-395, 397-399, 403-404, 409-410, 414-418, 421-423, 426-427, 432, 438-442, 445-446, 449, 451-452, 454-461, 463, 465, 467-468, 470-472, 474-600, 997-998, 1000-1007, 1009-1018, 1021-1025, 1027-1033, 1036, 1038-1042, 1044-1068, 1203-1242, 1244-1336, 764, 767-769, 774-776, 778, 782-783, 785-786, 788, 790-791, 795-796, 799-800, 803-806, 808-818, 821-826, 828-829, 831-833, 837, 840-841, 843-845, 847-848, 851-852, 857, 860-863, 866-867, 869-924 and 1365-1393.

In another preferred embodiment the amino acid sequence (polypeptide) is selected from the group consisting of Seq ID No 310-313, 320-321, 327-328, 331-332, 341, 344-347, 350-351, 354-356, 359, 380, 385, 396, 400, 405-407, 412, 424-425, 428-431, 437, 443, 462, 466, 469, 1008, 1020, 1026, 1035, 1043, 770-773, 779-780, 784, 793-794, 797-798, 801-802, 819, 830, 834, 838-839, 842, 849-850, 853-856 and 864.

In a further preferred embodiment the amino acid sequence (polypeptide) is selected from the group consisting of Seq ID No 301, 305-306, 318, 322, 325, 336, 338, 343, 365, 375, 381, 392, 401-402, 408, 411, 413, 419-420, 433-436, 444, 447-448, 450, 453, 464, 473, 999, 1019, 1034, 1037, 1243, 763, 765-766, 777, 781, 787, 789, 792, 807, 820, 827, 835-836, 846, 858-859, 865 and 868.

According to a further aspect the present invention provides fragments of hyperimmune serum-reactive antigens selected from the group consisting of peptides comprising amino acid sequences of column “predicted immunogenic aa” and “location of identified immunogenic region” of Table 1, 2, 3, 4 and 8, and the immunogenic epitopes-column “aa (start-stop)” of Table 6, the serum reactive epitope as defined in Table 7.

The present invention also provides a process for producing an enteroaggregative E. coli, enterotoxigenic E. coli, S. flexneri and/or C. jejuni hyperimmune serum reactive antigen or a fragment thereof according to the present invention comprising expressing one or more of the nucleic acid molecules according to the present invention in a suitable expression system.

Moreover, the present invention provides a process for producing a cell, which expresses an enteroaggregative E. coli, enterotoxigenic E. coli, S. flexneri and/or C. jejuni hyperimmune serum reactive antigen or a fragment thereof according to the present invention comprising transforming or transfecting a suitable host cell with the vector according to the present invention.

According to the present invention a pharmaceutical composition, especially a vaccine, comprising a hyperimmune serum-reactive antigen or a fragment thereof as defined in the present invention or a nucleic acid molecule as defined in the present invention is provided.

In a preferred embodiment the pharmaceutical composition further comprises an immunostimulatory substance, preferably selected from the group comprising polycationic polymers, especially polycationic peptides, immunostimulatory deoxynucleotides (ODNs), peptides containing at least two LysLeuLys motifs, especially KLKLKLK, neuroactive compounds, especially human growth hormone, alumn, Freund's complete or incomplete adjuvants or combinations thereof.

In a more preferred embodiment the immunostimulatory substance is a combination of either a polycationic polymer and immunostimulatory deoxynucleotides or of a peptide containing at least two LysLeuLys motifs and immunostimulatory deoxynucleotides.

In a still more preferred embodiment the polycationic polymer is a polycationic peptide, especially polyarginie.

According to the present invention the use of a nucleic acid molecule according to the present invention or a hyperimmune serum-reactive antigen or fragment thereof according to the present invention for the manufacture of a pharmaceutical preparation, especially for the manufacture of a vaccine against infection by enteroaggregative E. coli, enterotoxigenic E. coli, S. flexneri and/or C. jejune, is provided.

Also an antibody, or at least an effective part thereof, which binds at least to a selective part of the hyperimmune serum-reactive antigen or a fragment thereof according to the present invention, is provided herewith.

In a preferred embodiment the antibody is a monoclonal antibody.

In another preferred embodiment the effective part of the antibody comprises Fab fragments.

In a further preferred embodiment the antibody is a chimeric antibody.

In a still preferred embodiment the antibody is a humanized antibody.

The present invention also provides a hybridoma cell line, which produces an antibody according to the present invention.

Moreover, the present invention provides a method for producing an antibody according to the present invention, characterized by the following steps:
initiating an immune response in a non-human animal by administrating an hyperimmune serum-reactive antigen or a fragment thereof, as defined in the invention, to said animal,
removing an antibody containing body fluid from said animal, and
producing the antibody by subjecting said antibody containing body fluid to further purification steps.

Accordingly, the present invention also provides a method for producing an antibody according to the present invention, characterized by the following steps:
initiating an immune response in a non-human animal by administrating an hyperimmune serum-reactive antigen or a fragment thereof, as defined in the present invention, to said animal,
removing the spleen or spleen cells from said animal,
producing hybridoma cells of said spleen or spleen cells,
selecting and cloning hybridoma cells specific for said hyperimmune serum-reactive antigens or a fragment thereof,
producing the antibody by cultivation of, said cloned hybridoma cells and optionally further purification steps.

The antibodies provided or produced according to the above methods may be used for the preparation of a medicament for treating or preventing infections caused by enteroaggregative E. coli, enterotoxigenic E. coli, S. flexneri and/or C. jejuni.

According to another aspect the present invention provides an antagonist, which binds to a hyperimmune serum-reactive antigen or a fragment thereof according to the present invention.

Such an antagonist capable of binding to a hyperimmune serum-reactive antigen or fragment thereof according to the present invention may be identified by a method comprising the following steps:
a) contacting an isolated or immobilized hyperimmune serum-reactive antigen or a fragment thereof according to the present invention with a candidate antagonist under conditions to permit binding of said candidate antagonist to said hyperimmune serum-reactive antigen or fragment, in the presence of a component capable of providing a detectable signal in response to the binding of the candidate antagonist to said hyperimmune serum reactive antigen or fragment thereof; and
b) detecting the presence or absence of a signal generated in response to the binding of the antagonist to the hyperimmune serum reactive antigen or the fragment thereof.

An antagonist capable of reducing or inhibiting the interaction activity of a hyperimmune serum-reactive antigen or a fragment thereof according to the present invention to its interaction partner may be identified by a method comprising the following steps:
a) providing a hyperimmune serum reactive antigen or a hyperimmune fragment thereof according to the present invention,
b) providing an interaction partner to said hyperimmune serum reactive antigen or a fragment thereof, especially an antibody according to the present invention,
c) allowing interaction of said hyperimmune serum reactive antigen or fragment thereof to said interaction partner to form an interaction complex,
d) providing a candidate antagonist,
e) allowing a competition reaction to occur between the candidate antagonist and the interaction complex,
f) determining whether the candidate antagonist inhibits or reduces the interaction activities of the hyperimmune serum reactive antigen or the fragment thereof with the interaction partner.

The hyperimmune serum reactive antigens or fragments thereof according to the present invention may be used for the isolation and/or purification and/or identification of an interaction partner of said hyperimmune serum reactive antigen or fragment thereof.

The present invention also provides a process for in vitro diagnosing a disease related to expression of a hyperimmune serum-reactive antigen or a fragment thereof according to the present invention comprising determining the presence of a nucleic acid sequence encoding said hyperimmune serum reactive antigen or fragment thereof according to the present invention or the presence of the hyperimmune serum reactive antigen or fragment thereof according to the present invention.

The present invention also provides a process for in vitro diagnosis of a bacterial infection, especially a enteroaggregative E. coli, enterotoxigenic E. coli, S. flexneri and/or C. jejuni infection, comprising analyzing for the presence of a nucleic acid sequence encoding said hyperimmune serum reactive antigen or fragment thereof according to the present invention or the presence of the hyperimmune serum reactive antigen or fragment thereof according to the present invention.

Moreover, the present invention provides the use of a hyperimmune serum reactive antigen or fragment thereof according to the present invention for the generation of a peptide binding to said hyperimmune serum reactive antigen or fragment thereof, wherein the peptide is an anticaline.

The present invention also provides the use of a hyperimmune serum-reactive antigen or fragment thereof according to the present invention for the manufacture of a functional nucleic acid, wherein the functional nucleic acid is selected from the group comprising aptamers and spiegelmers.

The nucleic acid molecule according to the present invention may also be used for the manufacture of a functional ribonucleic acid, wherein the functional ribonucleic add is selected from the group comprising ribozymes, antisense nucleic acids and siRNA.

The present invention advantageously provides an efficient, relevant and comprehensive set of isolated nucleic acid molecules and their encoded hyperimmune serum reactive antigens or fragments thereof identified from enteroaggregative E. coli, enterotoxigenic E. coli, S. flexneri and/or C. jejuni using an antibody preparation from multiple human plasma pools and surface expression libraries derived from the genome of enteroaggregative E. coli, enterotoxigenic E. coli, S. flexneri and/or C. jejuni. Thus, the present invention fulfils a widely felt demand for enteroaggregative E. coli, enterotoxigenic E. coli, S. flexneri and/or C. jejuni antigens, vaccines, diagnostics and products useful in procedures for preparing antibodies and for identifying compounds effective against infections caused by enteroaggregative E. coli, enterotoxigenic E. coli, S. flexneri and/or C. jejuni.

An effective vaccine should be composed of proteins or polypeptides, which are expressed by all strains and are able to induce high affinity, abundant antibodies against cell surface components of enteroaggregative E. coli, enterotoxigenic E. coli, S. flexneri and/or C. jejuni. The antibodies should be IgG1 and/or IgG3 for opsonization, and any IgG subtype and IgA for neutralisation of adherence and toxin action. A chemically defined vaccine must be definitely superior compared to a whole cell vaccine (attenuated or killed), since components of enteroaggregative E. coli, enterotoxigenic E. coli, S. flexneri and/or C. jejuni, which cross-react with human tissues or inhibit opsonization can be eliminated, and the individual proteins inducing protective antibodies and/or a protective immune response can be selected.

The approach, which has been employed for the present invention, is based on the interaction of proteins or peptides encoded by enteroaggregative E. coli, enterotoxigenic E. coli, S. flexneri and/or C. jejuni with the antibodies present in human sera. The antibodies produced against enteroaggregative E. coli, enterotoxigenic E. coli, S. flexneri and/or C. jejuni by the human immune system and present in human sera are indicative of the in vivo expression of the antigenic proteins and their immunogenicity. In addition, the antigenic proteins as identified by the bacterial surface display expression libraries using pools of pre-selected sera, are processed in a second and third round of screening by individual selected or generated sera. Thus the present invention supplies an efficient, relevant, comprehensive set of antigens as a pharmaceutical composition, especially a vaccine preventing infections caused by enteroaggregative E. coli, enterotoxigenic E. coli, S. flexneri and/or C. jejuni.

In the antigen identification program for identifying a comprehensive set of antigens according to the present invention, at least two different bacterial surface expression libraries from each pathogen are screened with several serum pools or plasma fractions or other pooled antibody containing body fluids (antibody pools). The antibody pools are derived from a serum collection, which has been tested against antigenic compounds of enteroaggregative E. coli, enterotoxigenic E. coli, S. flexneri and/or C. jejuni, such as whole cell, total extracts and culture supernatant proteins. Preferably, two pools of sera (with 10 individual samples) are used. Sera determined to have high ELISA titter have to react with multiple proteins in immunoblotting in order to be considered hyperimmune and therefore relevant in the screening method applied for the present invention.

The expression libraries as used in the present invention should allow expression of all potential antigens, e.g. derived from all secreted and surface proteins of enteroaggregative E. coli, enterotoxigenic E. coli, S. flexneri and C. jejuni. Bacterial surface display libraries will be represented by a recombinant library of a bacterial host displaying a (total) set of expressed peptide sequences of enteroaggregative E. coli, enterotoxigenic E. coli, S. flexneri and C. jejuni on two selected outer membrane proteins (LamB and FhuA) at the bacterial host membrane {Georgiou, G., 1997}; {Etz, H. et al., 2001}. One of the advantages of using recombinant expression libraries is that the identified hyperimmune serum-reactive antigens may be instantly produced by expression of the coding sequences of the screened and selected dones expressing the hyperimmune serum-reactive antigens without further recombinant DNA technology or cloning steps necessary.

The comprehensive set of antigens identified by the described program according to the present invention is analysed further by one or more additional rounds of screening. Therefore individual antibody preparations or antibodies generated against selected peptides, which were identified as immunogenic are used. According to a preferred embodiment the individual antibody preparations for the second round of screening are derived from healthy adults and/or challenged adults who show an antibody titer above a certain minimum level, for example an antibody titer being higher than 80 percentile, preferably higher than 90 percentile, especially higher than 95 percentile of the human (patient or healthy individual) sera tested. Using such high titer individual antibody preparations in the second screening round allows a very selective identification of the hyperimmune serum-reactive antigens and fragments thereof from enteroaggregative E. coli, enterotoxigenic E. coli, S. flexneri and/or C. jejuni.

Following the comprehensive screening procedure, the selected antigenic proteins, expressed as recombinant proteins or in vitro translated products, in case it can not be expressed in prokaryotic expression systems, or the identified antigenic peptides (produced synthetically) are tested in a second screening by a series of ELISA and Western blotting assays for the assessment of their immunogenicity with a large human serum collection (minimum ˜150 healthy and patients sera).

It is important that the individual antibody preparations (which may also be the selected serum) allow a selective identification of the most promising candidates of all the hyperimmune serum-reactive antigens from all the promising candidates from the first round. Therefore, preferably at least 10 individual antibody preparations (i.e. antibody preparations (e.g. sera) from at least 10 different individuals having suffered from an infection to the chosen pathogen) should be used in identifying these antigens in the second screening round. Of course, it is possible to use also less than 10 individual preparations, however, selectivity of the step may not be optimal with a low number of individual antibody preparations. On the other hand, if a given hyperimmune serum-reactive antigen (or an antigenic fragment thereof) is recognized by at least 10 individual antibody preparations, preferably at least 30, especially at least 50 individual antibody preparations, identification of the hyperimmune serum-reactive antigen is also selective enough for a proper identification. Hyperimmune serum-reactivity may of course be tested with as many individual preparations as possible (e.g. with more than 100 or even with more than 1,000).

Therefore, the relevant portion of the hyperimmune serum-reactive antibody preparations according to the method of the present invention should preferably be at least 10, more preferred at least 30, especially at least 50 individual antibody preparations. Alternatively (or in combination) hyperimmune serum-reactive antigens may preferably be also identified with at least 20%, preferably at least 30%, especially at least 40% of all individual antibody preparations used in the second screening round.

According to a preferred embodiment of the present invention, the sera from which the individual antibody preparations for the second round of screening are prepared (or which are used as antibody preparations), are selected by their titer against enteroaggregative E. coli, enterotoxigenic E. coli, S. flexneri and/or C. jejuni (e.g. against a preparation of these pathogens, such as a lysate, cell wall components and recombinant proteins). Preferably, some are selected with a total IgA titer above 300 U, especially above 500 U, and/or an IgG titer above 5,000 U, especially above 10,000 U (U=units, calculated from the OD_{405 nm} reading at a given dilution) when the whole organism (total lysate or whole cells) is used as antigen in the ELISA.

The antibodies produced against streptococci by the human immune system and present in human sera are indicative of the in vivo expression of the antigenic proteins and their immunogenicity. The recognition of linear epitopes recognized by serum antibodies can be based on sequences as short as 4-5 amino acids. Of course it does not necessarily mean that these short peptides are capable of inducing the given antibody in vivo. For that reason the defined epitopes, polypeptides and proteins are further to be tested in animals (mainly in mice) for their capacity to induce antibodies against the selected proteins in vivo.

The preferred antigens are located on the cell surface or secreted, and are therefore accessible extracellularly. Antibodies against cell wall proteins are expected to serve multiple purposes: to inhibit adhesion, to interfere with nutrient acquisition, to inhibit immune evasion and to promote phagocytosis {Hornef, M. et al., 2002}. Antibodies against secreted proteins are beneficial in neutralisation of their function as toxin or virulence component. It is also known that bacteria communicate with each other through secreted proteins. Neutralizing antibodies against these proteins will interrupt growth-promoting cross-talk between or within infection causing pathogen species. Bioinformatic analyses (signal sequences, cell wall localisation signals, transmembrane domains) proved to be very useful in assessing cell surface localisation or secretion. The experimental approach includes the isolation of antibodies with the corresponding epitopes and proteins from human serum, and the generation of immune sera in mice against (poly) peptides selected by the bacterial surface display screens. These sera are then used in a third round of screening as reagents in at least one of the following assays: cell surface staining of enteroaggregative E. coli, enterotoxigenic E. coli, S. flexneri and C. jejuni grown under different conditions (FACS or microscopy), determination of neutralizing capacity (toxin, adherence), and promotion of opsonization and phagocytosis (in vitro phagocytosis assay).

For that purpose, bacterial E. coli clones are directly injected into mice and immune sera are taken and tested in the relevant in vitro assay for functional opsonic or neutralizing antibodies. Alternatively, specific antibodies may be purified from human or mouse sera using peptides or proteins as substrate.

Most pathogens enter at mucosal surfaces lining digestive, respiratory, and urino-reproductive tracts, which are collectively the largest immunologically active tissues in body, rendering the mucosal immune system the first line of defense. Host defense against enteric pathogenic bacteria relies mainly on the mucosal immune system, including intestinal secretory IgA, mucosal cellular immunity and toxin neutralisation. Vaccines against enteric pathogens should therefore stimulate both mucosal and humoral immunity, which combined provide good protective immunity against these pathogens. Antigens need to reach the gut associated lymphoid tissues (GALT; M-cells residing in the epithelium over Peyer's patches and lymphoid follicles), where they are recognized. Stimulation of these specific lymphocytes in the mucosal immune system and the resulting communication between the mucosal and serum systems leads to production of sIgA (secretory IgA), which is characteristic of a mucosal response and can for example prevent interaction of pathogens with receptors on mucosal surfaces. Inducing high affinity secretory antibodies by vaccination helps the immune system to eliminate bacteria and toxins. As the method according to the present invention uses antibodies from human serum, which were induced by previous encounters with the respective pathogens, it is an optimal tool for the identification of antigenic proteins from enteroaggregative E. coli, enterotoxigenic E. coli, S. flexneri and C. jejuni.

According to the antigen identification method used hereir, the present invention can surprisingly provide a set of comprehensive novel nucleic acids and novel hyperimmune serum reactive antigens and fragments thereof of enteroaggregative E. coli, enterotoxigenic E. coli, S. flexneri and C. jejuni, among other things, as described below. According to one aspect, the invention particularly relates to the nucleotide sequences encoding hyperimmune serum reactive antigens which sequences are set forth in the Sequence listing Seq ID No: 1-300, 601-762, 925-996 and 1069-1202 and the corresponding encoded amino acid sequences representing hyperimmune serum reactive antigens are set forth in the Sequence Listing Seq ID No 301-600, 763-924, 997-1068 and 1203-1336.

In a preferred embodiment of the present invention, a nucleic acid molecule is provided which exhibits 70% identity over their entire length to a nucleotide sequence set forth with Seq ID No 1-300, 601-762, 925-996 and 1069-1202. Most highly preferred are nucleic adds that comprise a region that is at least 80% or at least 85% identical over their entire length to a nucleic acid molecule set forth with Seq ID No 1-300, 601-762, 925-996 and 1069-1202. In this regard, nucleic acid molecules at least 90%, 91%, 92%, 93%, 94%, 95%, or 96% identical over their entire length to the same are particularly preferred. Furthermore, those with at least 97% are highly preferred, those with at least 98% and at least 99% are particularly highly preferred, with at least 99% or 99.5% being the more preferred, with 100% identity being especially preferred. Moreover, preferred embodiments in this respect are nucleic acids, which encode hyperimmune serum reactive antigens or fragments thereof (polypeptides) which retain substantially the same biological function or activity as the mature polypeptide encoded by said nucleic acids set forth in the Seq ID No 301-600, 763-924, 997-1068 and 1203-1336.

Identity, as known in the art and used herein, is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity can be readily calculated. While there exist a number of methods to measure identity between two polynucleotide or two polypeptide sequences, the term is well known to skilled artisans (e.g. Sequence Analysis in Molecular Biology, von Heinje, G., Academic Press, 1987). Preferred methods to determine identity are designed to give the largest match between the sequences tested. Methods to determine identity are codified in computer programs. Preferred computer program methods to determine identity between two sequences include, but are not limited to, GCG program package {Devereux, J. et al., 1984}, BLASTP, BLASTN, and PASTA {Altschul, S. et al, 1990}.

According to another aspect of the invention, nucleic acid molecules are provided which exhibit at least 96%, preferably at least 98%, expecially 100% identity to the nucleic acid sequence set forth with Seq ID No 10-13, 20-21, 27-28, 31-32, 41, 44-47, 50-51, 54-56, 59, 80, 85, 96, 100, 105-107, 112, 124-125, 128-131, 137, 143, 162, 166, 169, 936, 948, 954, 963, 971, 608-611, 617-618, 622, 631-632, 635-636, 639-640, 657, 668, 672, 676-677, 680, 687-688, 691-694 and 702.

According to a further aspect of the present invention, nucleic acid molecules are provided which are identical to the nucleic acid sequences set forth with Seq ID No 1, 5-6, 18, 22, 25, 36, 38,43, 65, 75, 81, 92, 101-102, 108, 111, 113, 119-120, 133-136, 144, 147-148, 150, 153, 164, 173, 927, 947, 962, 965, 1109, 601, 603-604, 615, 619, 625, 627, 630, 645, 658, 665, 673-674, 684, 696-697, 703 and 706.

The nucleic acid molecules according to the present invention can as a second alternative also be a nucleic acid molecule, which is at least essentially complementary to the nucleic add described as the first alternative above. As used herein complementary means that a nucleic acid strand is base pairing via Watson-Crick base pairing with a second nucleic acid strand. Essentially complementary as used herein means that the base pairing is not occurring for all of the bases of the respective strands but leaves a certain number or percentage of the bases unpaired or wrongly paired. The percentage of correctly pairing bases is preferably at least 70%, more preferably 80%, even more preferably 90% and most preferably any percentage higher than 90%. It is to be noted that a percentage of 70% matching bases is considered as homology and the hybridization having this extent of matching base pairs is considered as stringent Hybridization conditions for this kind of stringent hybridization may be taken from Current Protocols -in Molecular Biology (ohn Wiley and Sons, Inc., 1987). More particularly, the hybridization conditions can be as follows:
Hybridization performed e.g. in 5×SSPE, 5× Denhardt's reagent, 0.1% SDS, 100 g/mL sheared DNA at 68° C.
Moderate stringency wash in 0.2×SSC, 0.1% SDS at 42° C.
   High stringency wash in 0.1×SSC, 0.1% SDS at 68° C.

Genomic DNA with a GC content of 50% has an approximate T_{M} of 96° C. For 1% mismatch, the T_{M} is reduced by approximately 1° C.

In addition, any of the further hybridization conditions described herein are in principle applicable as well.

Of course, all nucleic acid sequence molecules which encode the same polypeptide molecule as those identified by the present invention are encompassed by any disclosure of a given coding sequence, since the degeneracy of the genetic code is directly applicable to unambiguously determine all possible nucleic acid molecules which encode a given polypeptide molecule, even if the number of such degenerated nucleic acid molecules may be high. This is also applicable for fragments of a given polypeptide, as long as the fragments encode a polypeptide being suitable to be used in a vaccination connection, e.g. as an active or passive vaccine.

The nucleic add molecule according to the present invention can as a third alternative also be a nucleic acid which comprises a stretch of at least 15 bases of the nucleic acid molecule according to the first and second alternative of the nucleic acid molecules according to the present invention as outlined above. Preferably, the bases form a contiguous stretch of bases. However, it is also within the scope of the present invention that the stretch consists of two or more moieties, which are separated by a number of bases.

The present nucleic acids may preferably consist of at least 20, even more preferred at least 30, especially at least 50 contiguous bases from the sequences disclosed herein. The suitable length may easily be optimized due to the planned area of use (e.g. as (PCR) primers, probes, capture molecules (e.g. on a (DNA) chip), etc.). Preferred nucleic acid molecules contain at least a contiguous 15 base portion of one or more of the predicted immunogenic amino acid sequences listed in tables 1 to 4. Specifically preferred are nucleic acids containing a contiguous portion of a DNA sequence of any sequence in the sequence protocol of the present application which shows 1 or more, preferably more than 2, especially more than 5, non-identical nucleic acid residues compared to the enteroaggregative E. coli, enterotoxigenic E. coli, S. flexneri 2a and C. jejuni NCTC 11168 genomes (NCBI accession: C. jejuni, NC 002163; S. flexneri 2a 301, NC 004337; ETEC & EAEC, no genome published) or the sequences listed in this patent and/or any other published genome sequence or parts thereof, (E. coli K12, NC 000913; E. coli O157:H7, NC 002695; S. flexneri 2a 2457T, NC 004741). Specifically preferred non-identical nucleic acid residues are residues, which lead to a non-identical amino acid residue. Preferably, the nucleic acid sequences encode polypeptides having at least 1, preferably at least 2, preferably at least 3 different amino acid residues compared to the published or listed enteroaggregative E. coli, enterotoxigenic E. coli, S. flexneri and C. jejuni counterparts mentioned above. Also such isolated polypeptides, being fragments of the proteins (or the whole protein) mentioned herein e.g. in the sequence listing, having at least 6, 7, or 8 amino acid residues and being encoded by these nudeic acids are preferred.

The nucleic acid molecule according to the present invention can as a fourth alternative also be a nucleic acid molecule which anneals under stringent hybridisation conditions to any of the nucleic acids of the present invention according to the above outlined first, second, and third alternative. Stringent hybridisation conditions are typically those described herein.

Finally, the nucleic acid molecule according to the present invention can as a fifth alternative also be a nucleic acid molecule which, but for the degeneracy of the genetic code, would hybridise to any of the nucleic acid molecules according to any nucleic acid molecule of the present invention according to the first, second, third, and fourth alternative as outlined above. This kind of nucleic acid molecule refers to the fact that preferably the nucleic acids according to the present invention code for the hyperimmune serum reactive antigens or fragments thereof according to the present inventions lTis kind of nucleic acid molecule is particularly useful in the detection of a nucleic acid molecule according to the present invention and thus the diagnosis of the respective microorganisms such as enteroaggregative E. coli, enterotoxigenic E. coli, S. flexneri and/or C. jejuni and any disease or diseased condition where these kinds of microorganims are involved. Preferably, the hybridisation would occur or be preformed under stringent conditions as described in connection with the fourth alternative described above.

Nucleic acid molecule as used herein generally refers to any ribonucleic acid molecule or deoxyribonucleic acid molecule, which may be unmodified RNA or DNA or modified RNA or DNA. Thus, for instance, nucleic acid molecule as used herein refers to, among other, single-and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term nucleic acid molecule includes DNAs or RNAs as described above that contain one or more modified bases. Thus, DNAs or RNAs with backbones modified for stability or for other reasons are “nucleic acid molecule” as that term is intended herein. Moreover, DNAs or RNAs comprising unusual bases, such as inosine, or modified bases, such as tritylated bases, to name just two examples, are nucleic acid molecule as the term is used herein. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic add molecule, as well as the chemical forms of DNA and RNA characteristic of viruses and cells, including simple and complex cells, inter alia. The term nucleic acid molecule also embraces short nucleic acid molecules often referred to as oligonucleotide(s). “Polynudeotide” and “nucleic acid” or “nucleic acid molecule” are often used interchangeably herein.

Nucleic acid molecules provided in the present invention also encompass numerous unique fragments, both longer and shorter than the nucleic acid molecule sequences set forth in the sequencing listing of the enteroaggregative E. coli, enterotoxigenic E. coli, S. flexneri and/or C. jejuni coding regions, which can be generated by standard cloning methods. To be unique, a fragment must be of sufficient size to distinguish it from other known nucleic acid sequences, most readily determined by comparing any selected enteroaggregative E. coli, enterotoxigenic E. coli, S. flexneri and/or C. jejuni fragment to the nucleotide sequences in computer databases such as GenBank.

Additionally, modifications can be made to the nucleic acid molecules and polypeptides that are encompassed by the present invention. For example, nucleotide substitutions can be made which do not affect the polypeptide encoded by the nucleic acid, and thus any nucleic add molecule which encodes a hyperimmune serum reactive antigen or fragments thereof is encompassed by the present invention.

Furthermore, any of the nucleic add molecules encoding hyperimmune serum reactive antigens or fragments thereof provided by the present invention can be functionally linked, using standard techniques such as standard cloning techniques, to any desired regulatory sequences, whether an enteroaggregative E. coli, enterotoxigenic E. coli, S. flexneri and/or C. jejuni regulatory sequence or a heterologous regulatory sequence, heterologous leader sequence, heterologous marker sequence or a heterologous coding sequence to create a fusion protein.

Nucleic add molecules of the present invention may be in the form of RNA, such as mRNA or cRNA, or in the form of DNA, including, for instance, cDNA and genomic DNA obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand.

The present invention further relates to variants of the herein above described nucleic acid molecules which encode fragments, analogs and derivatives of the hyperimmune serum reactive antigens and fragments thereof having a deducted enteroaggregative E. coli, enterotoxigenic E. coli, S. flexneri and/or C. jejuni amino acid sequence set forth in the Sequence Listing. A variant of the nucleic acid molecule may be a naturally occurring variant such as a naturally occurring allelic variant, or it may be a variant that is not known to occur naturally. Such non-naturally occurring variants of the nucleic acid molecule may be made by mutagenesis techniques, including those applied to nucleic add molecules, cells or organisms.

Among variants in this regard are variants that differ from the aforementioned nucleic acid molecules by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions. Preferred are nucleic acid molecules encoding a variant, analog, derivative or fragment, or a variant, analogue or derivative of a fragment, which have an enteroaggregative E. coli, enterotoxigenic E. coli, S. flexneri and/or C. jejuni sequence as set forth in the Sequence Listing, in which several, a few, 5 to 10, 1 to 5, 1 to 3, 2, 1 or no amino acid(s) is substituted, deleted or added, in any combination. Especially preferred among these are silent substitutions, additions and deletions, which do not alter the properties and activities of the enteroaggregative E. coli, enterotoxigenic E. coli, S. flexneri and/or C. jejuni polypeptides set forth in the Sequence Listing. Also especially preferred in this regard are conservative substitutions.

The peptides and fragments according to the present invention also include modified epitopes wherein preferably one or two of the amino acids of a given epitope are modified or replaced according to the rules disclosed in e.g. {Tourdot, S. et al, 2000}, as well as the nucleic acid sequences encoding such modified epitopes.

It is dear that also epitopes derived from the present epitopes by amino acid exchanges improving, conserving or at least not significantly impeding the T cell activating capability of the epitopes are covered by the epitopes according to the present invention. Therefore the present epitopes also cover epitopes, which do not contain the original sequence as derived from enteroaggregative E. coli, enterotoxigenic E. coli, S. flexneri and/or C. jejuni, but trigger the same or preferably an improved T cell response. These epitope are referred to as “heteroclitic”; they need to have a similar or preferably greater affinity to MHC/HLA molecules, and the need the ability to stimulate the T cell receptors (TCR) directed to the original epitope in a similar or preferably stronger manner.

Heteroclitic epitopes can be obtained by rational design i.e. taking into account the contribution of individual residues to binding to MHC/HLA as for instance described by {Rammensee, H. et al., 1999}, combined with a systematic exchange of residues potentially interacting with the TCR and testing the resulting sequences with T cells directed against the original epitope. Such a design is possible for a skilled man in the art without much experimentation.

Another possibility includes the screening of peptide libraries with T cells directed against the original epitope. A preferred way is the positional scanning of synthetic peptide libraries. Such approaches have been described in detail for instance by {Hemmer, B. et al., 1999} and the references given therein.

As an alternative to epitopes represented by the present derived amino acid sequences or heteroclitic epitopes, also substances mimicking these epitopes e.g. “peptidemimetica” or “retro-inverso-peptides” can be applied.

Another aspect of the design of improved epitopes is their formulation or modification with substances increasing their capacity to stimulate T cells. These include T helper cell epitopes, lipids or liposomes or preferred modifications as described in WO 01/78767.

Another way to increase the T cell stimulating capacity of epitopes is their formulation with immune stimulating substances for instance cytokines or chemokines like interleukin-2, -7, -12, -18, class I and II interferons (IFN), especially IFN-gamma, GM-CSF, TNF-alpha, flt3-ligand and others.

As discussed additionally herein regarding nucleic acid molecule assays of the invention, for instance, nucleic acid molecules of the invention as discussed above, may be used as a hybridization probe for RNA, cDNA and genomic DNA to isolate full-length cDNAs and genomic clones encoding polypeptides of the present invention and to isolate cDNA and genomic clones of other genes that have a high sequence similarity to the nucleic acid molecules of the present invention. Such probes generally will comprise at least 15 bases. Preferably, such probes will have at least 20, at least 25 or at least 30 bases, and may have at least 50 bases. Particularly preferred probes will have at least 30 bases, and will have 50 bases or less, such as 30, 35, 40, 45, or 50 bases.

For example, the coding region of a nucleic acid molecule of the present invention may be isolated by screening a relevant library using the known DNA sequence to synthesize an oligonucleotide probe. A labeled oligonucleotide having a sequence complementary to that of a gene of the present invention is then used to screen a library of cDNA, genomic DNA or mRNA to determine to which members of the library the probe hybridizes.

The nucleic acid molecules and polypeptides of the present invention may be employed as reagents and materials for development of treatments of and diagnostics for disease, particularly human disease, as further discussed herein relating to nucleic acid molecule assays, inter alia.

The nucleic acid molecules of the present invention that are oligonucleotides can be used in the processes herein as described, but preferably for PCR, to determine whether or not the enteroaggregative E. coli, enterotoxigenic E. coli, S. flexneri and/or C. jejuni genes identified herein in whole or in part are present and/or transcribed in infected tissue such as blood. It is recognized that such sequences will also have utility in diagnosis of the stage of infection and type of infection the pathogen has attained. For this and other purposes the arrays comprising at least one of the nucleic acids according to the present invention as described herein, may be used.

The nucleic acid molecules according to the present invention may be used for the detection of nucleic acid molecules and organisms or samples containing these nucleic adds. Preferably such detection is for diagnosis, more preferable for the diagnosis of a disease related or linked to the present or abundance of enteroaggregative E. coli, enterotoxigenic E. coli, S. flexneri and/or C. jejuni.

Eukaryotes (herein also “individual(s)”), particularly mammals, and especially humans, infected with enteroaggregative E. coli, enterotoxigenic E. coli, S. flexneri and/or C. jejuni may be identifiable by detecting any of the nucleic acid molecules according to the present invention detected at the DNA level by a variety of techniques. Preferred nucleic add molecules candidates for distinguishing enteroaggregative E. coli, enterotoxigenic E. coli, S. flexneri and/or C. jejuni from other organisms can be obtained.

The invention provides a process for diagnosing disease, arising from infection with enteroaggregative E. coli, enterotoxigenic E. coli, S. flexneri and/or C. jejune, comprising determining from a sample isolated or derived from an individual an increased level of expression of a nucleic acid molecule having the sequence of a nucleic acid molecule set forth in the Sequence Listing. Expression of nucleic acid molecules can be measured using any one of the methods well known in the art for the quantitation of nucleic acid molecules, such as, for example, PCR, RT-PCR, Rnase protection, Northern blotting, other hybridisation methods and the arrays described herein.

Isolated as used herein means separated “by the hand of man” from its natural state; i.e., that, if it occurs in nature, it has been changed or removed from its original environment, or both. For example, a naturally occurring nucleic acid molecule or a polypeptide naturally present in a living organism in its natural state is not “isolated,” but the same nucleic acid molecule or polypeptide separated from the coexisting materials of its natural state is “isolated”, as the term is employed herein. As part of or following isolation, such nucleic acid molecules can be joined to other nudeic add molecules, such as DNAs, for mutagenesis, to form fusion proteins, and for propagation or expression in a host, for instance. The isolated nucleic acid molecules, alone or joined to other nucleic acid molecules such as vectors, can be introduced into host cells, in culture or in whole organisms. Introduced into host cells in culture or in whole organisms, such DNAs still would be isolated, as the term is used herein, because they would not be in their naturally occurring form or environment. Similarly, the nucleic acid molecules and polypeptides may occur in a composition, such as a media formulations, solutions for introduction of nucleic add molecules or polypeptides, for example, into cells, compositions or solutions for chemical or enzymatic reactions, for instance, which are not naturally occurring compositions, and, therein remain isolated nucleic acid molecules or polypeptides within the meaning of that term as it is employed herein.

The nucleic acids according to the present invention may be chemically synthesized. Alternatively, the nucleic acids can be isolated from enteroaggregative E. coli, enterotoxigenic E. coli, S. flexneri and/or C. jejuni by methods known to the one skilled in the art.

According to another aspect of the present invention, a comprehensive set of novel hyperimmune serum reactive antigens and fragments thereof are provided by using the herein described antigen identification method. In a preferred embodiment of the invention, a hyperimmune serum-reactive antigen comprising an amino acid sequence being encoded by any one of the nucleic acids molecules herein described and fragments thereof are provided. In another preferred embodiment of the invention a novel set of hyperimmune serun-reactive antigens which comprises amino acid sequences selected from a group consisting of the polypeptide sequences as represented in Seq ID No 302-304, 307-309, 314-317, 319, 323-324, 326, 329-330, 333-335, 339-340, 342, 348-349, 352-353, 357-358, 360-364, 366-374, 376-379, 382-384, 386-391, 393-395, 397-399, 403-404, 409-410, 414-418, 421-423, 426-427, 432, 438-442, 445-446, 449, 451-452, 454-461, 463, 465, 467-468, 470-472, 474-600, 997-998, 1000-1007, 1009-1018, 1021-1025, 1027-1033, 1036, 1038-1042, 1044-1068, 1203-1242, 1244-1336, 764, 767-769, 774-776, 778, 782-783, 785-786, 788, 790-791, 795-796, 799-800, 803-806, 808-818, 821-826, 828-829, 831-833, 837, 840-841, 843-845, 847-848, 851-852, 857, 860-863, 866-867, 869-924 and 1365-1393 and fragments thereof are provided. In a further preferred embodiment of the invention hyperimmune serum-reactive antigens, which comprise amino acid sequences selected from a group consisting of the polypeptide sequences as represented in Seq ID No 310-313, 320-321, 327-328, 331-332, 341, 344-347, 350-351, 354-356, 359, 380, 385, 396, 400, 405-407, 412, 424-425, 428-431, 437, 443, 462, 466, 469, 1008, 1020, 1026, 1035, 1043, 770-773, 779-780, 784, 793-794, 797-798, 801-802, 819, 830, 834, 838-839, 842, 849-850, 853-856 and 864 and fragments thereof are provided. In a still preferred embodiment of the invention hyperimmune serum-reactive antigens, which comprise amino acid sequences selected from a group consisting of the polypeptide sequences as represented in Seq ID No 301, 305-306, 318, 322, 325, 336, 338, 343, 365, 375, 381, 392, 401-402, 408, 411, 413, 419-420, 433-436, 444, 447-448, 450, 453, 464, 473, 999, 1019, 1034, 1037, 1243, 763, 765-766, 777, 781, 787, 789, 792, 807, 820, 827, 835-836, 846, 858-859, 865 and 868 and fragments thereof are provided.

The hyperimmune serum reactive antigens and fragments thereof as provided in the invention include any polypeptide set forth in the Sequence Listing as well as polypeptides whidh have at least 70% identity to a polypeptide set forth in the Sequence Listing, preferably at least 80% or 85% identity to a polypeptide set forth in the Sequence Listing, and more preferably at least 90% similarity (more preferably at least 90% identity) to a polypeptide set forth in the Sequence Listing and still more preferably at least 95%, 96%, 97%, 98%, 99% or 99.5% similarity (still more preferably at least 95%, 96%, 97%, 98%, 99%, or 99.5% identity) to a polypeptide set forth in the Sequence Listing and also include portions of such polypeptides with such portion of the polypeptide generally containing at least 4 amino acids and more preferably at least 8, still more preferably at least 30, still more preferably at least 50 amino acids, such as 4, 8, 10, 20, 30, 35, 40, 45 or 50 amino acids.

The invention also relates to fragments, analogs, and derivatives of these hyperimmune serum reactive antigens and fragments thereof. The terms “fragment”, “derivative” and “analog” when referring to an antigen whose amino add sequence is set forth in the Sequence Listing, means a polypeptide which retains essentially the same or a similar biological function or activity as such hyperimmune serum reactive antigen and fragment thereof.

The fragment, derivative or analog of a hyperimmune serum reactive antigen and fragment thereof may be 1) one in which one or more of the amino add residues are substituted with a conserved or non-conserved amino acid residue (preferably a conserved amino acid residue) and such substituted amino acid residue may or may not be one encoded by the genetic code, or 2) one in which one or more of the amino acid residues includes a substituent group, or 3) one in which the mature hyperimmune serum reactive antigen or fragment thereof is fused with another compound, such as a compound to increase the half-life of the hyperimmune serum reactive antigen and fragment thereof (for example, polyethylene glycol), or 4) one in which the additional amino acids are fused to the mature hyperimmune serum reactive antigen or fragment thereof, such as a leader or secretory sequence or a sequence which is employed for purification of the mature hyperimmune serum reactive antigen or fragment thereof or a proprotein sequence. Such fragments, derivatives and analogs are deemed to be within the scope of those skilled in the art from the teachings herein.

The present invention also relates to antigens of different enteroaggregative E. coli, enterotoxigenic E. coli, S. flexneri and/or C. jejuni isolates. Such homologues may easily be isolated based on the nucleic acid and amino acid sequences disclosed herein. There are multiple serotypes or clinical strains distinguished to date for each of the pathogens and the typing is based on serotype specific antisera or molecular approaches. The presence of any antigen can accordingly be determined for every serotype. In addition, it is possible to determine the variability of a particular antigen in the various serotypes as described for the S. pyogenes sic gene {Hoe, N. et al., 2001}. The contribution of the various serotypes to the different diarrheal infections varies in different age groups and especially geographical regions. It is an important aspect that the most valuable protective antigens need to be conserved among various clinical strains.

Among the particularly preferred embodiments of the invention in this regard are the hyperimmune serum reactive antigens set forth in the Sequence Listing, variants, analogs, derivatives and fragments thereof, and variants, analogs and derivatives of fragments. Additionally, fusion polypeptides comprising such hyperimmune serum reactive antigens, variants, analogs, derivatives and fragments thereof, and variants, analogs and derivatives of the fragments are also encompassed by the present invention. Such fusion polypeptides and proteins, as well as nucleic add molecules encoding them, can readily be made using standard techniques, including standard recombinant techniques for producing and expression of a recombinant polynucleic add encoding a fusion protein.

Among preferred variants are those that vary from a reference by conservative amino acid substitutions. Such substitutions are those that substitute a given amino acid in a polypeptide by another amino acid of like characteristics. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr.

Further particularly preferred in this regard are variants, analogs, derivatives and fragments, and variants, analogs and derivatives of the fragments, having the amino acid sequence of any polypeptide set forth in the Sequence Listing, in which several, a few, 5 to 10, 1 to 5, 1 to 3, 2, 1 or no amino acid residues are substituted, deleted or added, in any combination Especially preferred among these are silent substitutions, additions and deletions, which do not alter the properties and activities of the polypeptide of the present invention. Also especially preferred in this regard are conservative substitutions. Most highly preferred are polypeptides having an amino acid sequence set forth in the Sequence Listing without substitutions.

The hyperimmune serum reactive antigens and fragments thereof of the present invention are preferably provided in an isolated form, and preferably are purified to homogeneity.

Also among preferred embodiments of the present invention are polypeptides comprising fragments of the polypeptides having the amino acid sequence set forth in the Sequence Listing, and fragments of variants and derivatives of the polypeptides set forth in the Sequence Listing.

In this regard a fragment is a polypeptide having an amino acid sequence that entirely is the same as part but not all of the amino acid sequence of the afore mentioned hyperimmune serum reactive antigen and fragment thereof, and variants or derivative, analogs, fragments thereof Such fragments may be “free-standing”, i.e., not part of or fused to other amino acids or polypeptides, or they may be comprised within a larger polypeptide of which they form a part or region. Also preferred in this aspect of the invention are fragments characterised by structural or functional attributes of the polypeptide of the present invention, i.e. fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta-amphipathic regions, flexible regions, surface-forming regions, substrate binding regions, and high antigenic index regions of the polypeptide of the present invention, and combinations of such fragments. Preferred regions are those that mediate activities of the hyperimmune serum reactive antigens and fragments thereof of the present invention. Most highly preferred in this regard are fragments that have a chemical, biological or other activity of the hyperimmune serum reactive antigen and fragments thereof of the present invention, including those with a similar activity or an improved activity, or with a decreased undesirable activity. Particularly preferred are fragments comprising receptors or domains of enzymes that confer a function essential for viability of enteroaggregative E. coli, enterotoxigenic E. coli, S. flexneri and/or C. jejuni or the ability to cause disease in humans. Further preferred polypeptide fragments are those that comprise or contain antigenic or immunogenic determinants in an animal, especially in a human.

An antigenic fragment is defined as a fragment of the identified antigen, which is for itself antigenic or may be made antigenic when provided as a hapten. Therefore, also antigens or antigenic fragments showing one or (for longer fragments) only a few amino acid exchanges are enabled with the present invention, provided that the antigenic capacities of such fragments with amino acid exchanges are not severely deteriorated on the exchange(s), i.e., suited for eliciting an appropriate immune response in an individual vaccinated with this antigen and identified by individual antibody preparations from individual sera.

Preferred examples of such fragments of hyperimmune serum-reactive antigens selected from the group consisting of peptides comprising amino acid sequences of column “predicted immunogenic aa” and “location of identified immunogenic region” of Table 1 to 4 and Table 8, “aa (start-stop)” of Table 6, the serum reactive epitope as defined in Table 7, especially peptides comprising amino add 12-19, 24-29, 37-43, 47-53, 65-72, 83-95, 112-122, 136-147, 162-168, 174-181, 189-195, 201-208, 216-221, 234-243, 270-276, 278-288, 305-316, 318-342, 350-356, 368-400, 420-428, 434-443, 471-477, 481-488, 530-535, 540-547, 566-575, 591-601, 603-609, 624-629 and 192-333 of Seq ID No 301; 9-22, 38-46, 51-61, 66-73, 108-126, 136-154, 162-169, 177-186, 198-204, 231-254, 256-272, 277-295, 297-311, 314-328, 331-338, 379-385, 393-402 and 69-88 of Seq ID No 302; 18-36, 43-53, 80-86, 94-110, 112-118, 152-168, 170-182, 188-198, 200-220, 225-230, 237-243, 248-259, 265-289, 298-317, 325-331, 338-344, 349-360, 382-389, 400-407, 413-419, 433-453, 494-501, 503-524, 530-536, 557-565, 574-582, 586-592, 603-629, 631-637, 643-657, 673-680, 699-705, 715-720, 754, 764-771, 778-793, 800-844, 853-858, 874-893, 899-905, 915-929, 957-965, 134-148, 525-552 and 821-920 of Seq ID No 303; 11-22, 28-34, 40-45, 65-86, 99-107, 115-125, 132-141, 143-150, 158-190, 203-211, 216-239, 246-257, 259-270, 272-279, 286-306, 313-332, 338-364, 369-380, 387-397, 410-418, 422-435, 449-455, 467-510, 515-521, 532-538, 547-563, 251-323 and 368-389 of Seq ID No 304; 7-20, 28-45, 51-66, 81-104, 108-115, 124-137, 149-155, 161-206, 209-214, 222-239, 250-262, 276-282, 309-343, 351-363, 365-386, 405-413, 435-440, 446-454, 458-466, 470-477, 482-492 and 235-269 of Seq ID No 305; 12-43, 51-60, 65-74, 76-86, 102-108, 110-118, 129-139, 146-160, 164-172, 180-192, 195-208, 212-220, 228-250, 252-267, 271-277, 281-288, 296-313, 344-353, 364-379, 381-387, 394-414, 435-443, 451-460, 468-474, 484-491, 500-510, 541-556, 560-586, 604-618, 635-641, 647-657, 668-705, 715-727, 729-734, 740-745, 760-780, 803-809 and 752-825 of Seq ID No 306; 16-23, 66-90, 98-110, 125-131, 144-150, 194-200, 213-219, 221-232, 237-256, 263-281, 293-298, 311-318, 326-337, 339-354, 373-389, 396-402, 404-421, 427-439, 441-448, 452-462, 467-479, 508-530, 534-541, 544-550, 562-569, 575-581, 583-592, 595-628, 636-656, 658-672, 674-680, 687-697, 715-721, 731-736, 739-749, 754-761, 771-788, 790-797, 813-824 and 623-653 of Seq ID No 307; 14-42, 51-57, 66-77, 84-96, 103-111, 129-148, 158-193, 198-208, 212-222, 242-262 and 31-133 of Seq ED No 308; 4-23, 36-62, 65-84, 98-104, 128-135, 144-161, 175-204, 219-240, 250-264, 266-278, 280-290 and 119-152 of Seq ID No 309; 13-26, 33-45, 50-60, 75-81, 97-105, 123-131, 138-145, 158-166, 168-177 and 66-170 of Seq ID No 310; 20-26, 35-50, 52-67, 85-100, 106-149, 189-199, 202-208, 217-226, 236-244, 270-294, 310-332, 340-347, 350-356, 364-373, 375-380, 401-428, 438-445, 477-493, 513-548, 555-564, 568-596, 600-612, 650-665, 667-674, 680-687, 696-707, 715-722, 728-764, 779-784, 795-809, 813-820, 837-843, 858-864, 885-891, 894-900, 907-917, 922-935, 941-946, 970-977, 979-986, 1022-1032 and 881-924 of Seq ID No 311; 13-21, 48-57, 72-83, 105-119, 125-133, 146-153, 170-177, 221-239, 245-274, 283-292, 299-305, 317-329, 335-343, 358-367, 374-380, 399-407, 430-438, 449-454, 473-479, 483-505, 517-527, 531-537, 554-560, 586-599, 601-616, 623-629, 639-647, 649-654, 658-667, 669-676, 690-709, 714-729 and 277-306 of Seq ID No 312; 14-28, 34-40, 45-54, 69-83, 86-100, 116-123, 135-143, 146-161, 168-179, 187-200, 203-225, 237-250, 255-265, 271-292, 298-314 and 104-138 of Seq ID No 313; 4-28, 36-42, 78-85, 106-122, 130-135, 144-150, 161-175, 180-190, 194-200, 226-234, 256-265, 274-294, 309-316, 324-333, 373-379, 382-389, 398-404, 407-416, 422-446, 451-462, 530-541 and 448-483 of Seq ID No 314; 5-50, 53-64, 75-80, 110-116, 119-125, 131-148, 150-156, 192-217, 224-229, 260-267, 275-281, 283-300, 305-310, 323-343, 358-365, 400-406, 422-429, 447-464, 498-503, 512-518, 520-526, 530-537, 552-562, 614-623, 697-703, 705-711, 719-727, 729-743, 745-753, 788-796, 802-810, 813-834 and 377-400 of Seq ID No 315; 4-26, 55-61, 64-70, 74-99, 107-119, 128-134, 137-154, 167-178 and 108-137 of Seq ID No 316; 12-54, 70-76, 117-125, 135-143, 196-202, 205-213, 243-261, 263-269, 278-298, 312-318, 320-326, 334-346, 358-368, 377-389, 396-404, 414-423, 429-438, 448-455, 483-490, 540-549, 557-563, 592-599, 601-609, 622-628, 632-653, 656-692, 695-712, 714-730, 760-766, 775-786, 788-803, 807-814, 820-831, 848-854, 875-886, 892-899, 911-917, 919-925, 927-935, 954-974, 980-992, 1017-1026, 1032-1044, 1074-1079, 1082-1089, 1098-1108, 1115-1120, 1129-1137, 1139-1145, 1161-1170, 1189-1195, 1197-1212, 1219-1225, 1234-1250, 1267-1283, 1302-1318, 1321-1329, 1362-1368, 1377-1388, 1395-1408 and 894-924 of Seq ID No 317; 58-76, 82-87, 95-103, 107-114, 122-136, 139-148, 150-162, 165-172, 184-190, 203-220, 228-238, 245-258, 260-267, 288-295, 299-328, 333-352, 357-386, 424-429 and 2-20 of Seq ID No 318; 5-26, 59-66, 68-74, 81-87, 105-116, 122-132, 144-160, 185-212, 217-223, 228-234, 241-252, 269-274, 291-313, 318-326, 335-342, 352-358, 368-375, 397-404, 411-422, 431-439, 458-472, 474-485, 493-499, 509-519, 521-527, 530-558, 563-579, 590-601 and 554-596 of Seq ID No 319; 6-12, 18-28, 39-45, 69-102, 120-128, 137-147, 164-170, 173-179, 223-230, 236-254, 265-277, 320-326, 350-368, 376-400, 404-416, 441-448, 462-477 and 178-285 of Seq ID No 320; 4-19, 21-47, 52-57, 59-73, 79-86, 88-95, 100-108, 114-129, 136-143, 145-151, 176-182, 235-242, 248-258, 273-281, 301-308, 310-316, 329-340, 347-354, 363-380, 384-400, 407-415, 430-441, 469-480, 491-504, 507-526, 530-540, 547-554, 563-579, 609-617, 620-626, 630-636, 643-655, 665-680, 706-714, 718-725, 729-740, 747-754, 756-779, 790-803, 806-816, 818-824, 829-840, 842-853, 862-877, 901-912, 928-939, 941-952, 961-978, 988-999, 1026-1037, 1067-1078, 1080-1087, 1089-1098, 1104-1115, 1117-1124, 1128-1139, 1143-1151, 1155-1176, 1180-1186, 1205-1211, 1218-1224, 1228-1242, 1244-1251, 1258-1278, 1280-1287, 1290-1298, 1304-1326, 1331-1341, 1363-1378, 1392-1399, 1407-1415, 1430-1443, 1445-1454, 179-265 and 1343-1368 of Seq ID No 321; 4-24, 31-37, 61-75, 83-89, 94-102, 117-123, 130-143, 184-191, 203-210, 212-228, 270-284, 286-292, 301-307, 312-319, 329-335 and 238-319 of Seq ID No 322; 4-16, 22-35, 39-44, 50-59, 65-73, 86-104, 108-117, 128-137, 139-147, 153-159, 165-171, 185-192, 198-223 and 102-120 of Seq ID No 323; 8-16, 23-30, 32-40, 45-50, 61-68, 81-89, 91-114, 121-129, 131-149, 161-185, 191-200, 217-224, 229-249, 253-262, 266-273, 282-289, 297-303 and 265-282 of Seq ID No 324; 10-48, 64-71, 81-88, 100-112, 130-140, 153-170, 177-184, 197-202, 236-250, 266-272, 284-292, 294-300, 306-318, 320-326, 346-357, 379-387, 389-396, 405-416, 424-435, 447-453, 474-483, 501-510, 529-536, 550-568, 582-594, 606-611, 625-631, 633-645, 664-672, 685-692, 703-711, 730-745, 761-775, 782-790, 792-804, 816-825, 827-834, 840-866 and 178-193 of Seq ID No 325; 11-25, 39-57, 69-94, 100-107, 118-155, 158-171, 189-201, 226-233, 236-245, 249-263, 268-277, 287-312, 315-329, 333-342, 351-357, 364-374, 382-388, 399-407, 419-449, 454-471, 486-492, 494-504, 515-541, 547-552, 578-600, 611-623, 625-641, 651-657, 678-692, 699-709, 713-720, 746-752, 772-781, 791-801, 829-844, 880-893, 900-910, 915-923, 936-942, 953-970, 88-167 and 804-839 of Seq ID No 326; 6-16, 1940, 55-61, 67-92, 103-108, 125-142, 154-162, 190-196, 236-247, 257-272, 280-290, 310-320, 331-343, 367-375, and 281-297 of Seq ID No 327; 42-48, 63-116, 151-172, 181-187, 189-196, 200-205, 217-232, 256-261, 288-319, 326-352, 360-374, 380-404, 412-435, 453-466, 474-485, 500-507, 514-519, 525-530, 558-564, 568-582, 590-595, 600-610, 633-640, 666-671, 694-702, 715-722, 730-737, 757-768, 774-785, 820-827, 832-848, 873-878, 883-890, 906-915, 918-930, 937-944, 948-956, 960-966, 970-978, 1023-1040, 1049-1056, 1065-1071, 1085-1090, 1105-1117, 1122-1132, 1165-1171, 1186-1195, 1210-1216, 1309-1342, 1345-1352, 1354-1360, 1375-1385, 1400-1407, 1414-1421, 1430-1439, 1446-1467, 1479-1485, 1522-1530, 1565-1572, 1577-1586, 1596-1608 and 717-741 of Seq ID No 328; 5-11, 17-24, 26-32, 36-43, 50-61, 67-73, 91-102, 111-126, 133-148, 154-161, 167-173, 179-195, 208-223, 230-252, 270-286, 292-306, 308-347, 352-371, 373-380, 386-395, 404-410, 418-431, 436-444, 447-460, 463-477, 486-492, 522-533, 545-553 and 36-133 of Seq ID No 329; 4-23, 68-78, 100-107, 135-149, 152-159 and 1-88 of Seq ID No 330; 5-12, 18-27, 35-55, 68-95, 100-109, 117-122, 129-135, 157-162 and 37-98 of Seq ID No 331; 5-52, 64-80, 86-106, 108-155, 175-190, 223-231, 234-248 and 53-70 of Seq ID No 332; 25-46, 59-64, 69-75, 83-90, 93-100, 107-115, 124-135, 151-177, 183-189, 194-206, 209-215, 219-224, 251-263, 267-276, 305-311, 318-327, 332-338, 350-356, 380-396, 406-412, 414-423, 431-437, 453-461, 463-481, 483-491, 505-510, 513-523, 528-545, 568-575 and 226-275 of Seq ID No 333; 5-29, 37-43, 47-54, 61-70 and 31-65 of Seq ID No 334; 10-35, 42-59, 65-70, 76-85, 92-104, 149-155, 184-191, 234-243, 248-259, 268-277, 383-389, 391-398, 410-430, 445-454, 488-504, 518-523, 530-538, 574-590, 615-623, 627-633, 652-660, 662-670, 674-683, 703-714, 720-728, 731-737, 751-757 and 547-572 of Seq ID No 335; 5-12, 39-51, 57-64, 67-84, 86-108, 124-130, 138-159, 167-179, 181-202, 226-235 and 133-214 of Seq ID No 336; 12-20, 29-40, 57-77, 79-88, 97-103, 111-117, 119-137, 174-200, 202-218, 221-229, 231-238, 240-246, 254-264, 266-280, 296-308, 321-331 and 23-54 of Seq ID No 337, 7-17, 19-54, 66-101, 114-133, 146-182, 193-210, 219-226, 232-238, 244-250, 253-261, 266-279 and 1-31 of Seq ID No 338; 4-11, 17-32, 38-58, 68-111, 113-130, 132-186, 200-212, 219-227, 240-249, 256-265, 270-278, 285-305, 311-317, 328-341, 343-351, 373-386, 389-414, 419-433, 439-504, 510-535, 553-564, 588-594, 599-604, 609-618, 620-631, 635-657, 664-670, 684-703, 705-717 and 661-695 of Seq ID No 339; 5-33, 67-78, 122-129, 141-150, 172-185, 201-209, 217-223, 235-252, 289-295, 303-316, 355-368, 383-389, 398-406, 426-437, 445-451, 459-467, 479-496, 512-517, 523-530, 535-562, 577-584, 590-605, 610-616, 618-632, 644-654, 663-669, 680-688, 70-85 and 271-403 of Seq ID No 340; 4-53, 55-62 and 59-71 of Seq ID No 341; 26-38, 43-63, 67-76, 78-98, 105-112, 115-121, 132-144, 148-153, 179-184, 194-203, 239-245, 261-278, 282-315 and 231-283 of Seq ID No 342; 13-22, 24-30, 49-61, 65-72, 90-97, 99-105, 115-131, 152-160, 165-171, 176-188, 202-221, 231-250, 255-274, 280-286, 288-296, 331-337, 339-347, 350-358, 374-385, 391-408, 418-427, 438-453, 468-476, 482-490, 497-506, 526-532, 534-583, 696-702, 713-719, 730-748, 750-758, 762-776, 802-808, 825-857, 864-950, 963-1004, 1015-1023, 1046-1058 and 571-665 of Seq ID No 343; 5-13, 18-24, 29-45, 51-58, 78-85, 87-94, 109-117, 122-128, 146-161, 175-189 and 1-83 of Seq ID No 344; 5-17, 40-46, 50-65, 73-86, 89-98, 114-139, 151-157, 165-173, 186-195, 197-213, 215-227, 245-260, 310-315, 364-369, 405-415, 428-436, 456-464, 471-482, 507-514, 518-531, 539-565, 648-654, 681-687, 690-707, 720-731, 743-756, 764-771, 801-806, 815-828, 830-836, 204-283 and 287-363 of Seq ID No 345; 6-27, 33-40, 62-78, 83-89, 91-99, 102-109, 115-134, 150-156, 160-170, 172-204, 233-244 and 109-127 of Seq ID No 346; 4-11, 17-25, 31-38, 55-80, 105-113, 115-123, 160-171, 195-201, 210-222, 254-264, 290-296, 301-310, 315-321, 329-334, 349-358, 377-394, 403-409, 424-432, 452-460, 466-474, 480-495, 510-516, 527-539, 554-562, 568-579, 587-592, 599-608, 627-635, 637-646, 661-668, 702-709, 723-736, 744-766, 768-778, 785-802, 815-821, 828-835, 862-868, 876-888, 892-906, 908-918, 921-950, 192-275 and 394-430 of Seq ID No 347; 18-26, 33-78, 80-87, 120-128, 141-182, 184-208, 251-257, 269-300, 305-311 and 208-230 of Seq ID No 348; 11-72, 84-91, 99-109, 112-120, 143-155, 162-183, 188-197, 222-231 and 30-176 of Seq ID No 349; 4-17, 41-56, 61-67, 74-109, 142-149, 158-185, 193-210, 216-236, 241-249 and 84-162 of Seq ID No 350; 845, 135-140, 176-182, 189-196, 206-216, 218-235, 260-269, 272-278, 307-313, 331-344, 352-359, 371-395, 403-414, 416-422, 426-438, 451-470, 478-484, 493-502, 504-511, 514-525, 527-534 and 104-180 of Seq ID No 351; 6-25, 49-59, 65-94, 107-115, 117-124, 135-151, 176-185, 203-209 and 145-173 of Seq ID No 352; 5-15, 46-56, 58-81, 83-111, 118-138, 146-158, 165-175 and 946 of Seq ID No 353; 7-15, 36-43, 54-60, 65-73, 88-94, 107-113, 122-128, 134-141, 162-171, 182-216, 218-235, 249-258, 266-278, 290-301, 308-338, 362-368 and 100-141 of Seq ID No 354; 4-14, 19-24, 27-36, 38-51, 59-73, 90-96, 102-121, 138-150, 157-174, 176-202, 212-225, 229-241, 250-258, 261-268, 279-291, 293-310, 319-338, 358-368, 371-389, 393-398, 404-413, 416-433, 435-442, 458-471 and 327-355 of Seq ID No 355; 10-15, 32-49, 61-69, 97-103, 128-134, 143-154, 164-179 and 99-124 of Seq ID No 356; 6-37, 40-48, 56-68, 108-127, 135-141, 145-163, 170-179, 207-216, 224-234, 238-244, 249-261, 266-283, 352-363, 371-378, 380-392, 447-454, 468-489, 497-503, 505-510 and 434-465 of Seq ID No 357; 6-33, 46-68, 78-84, 120-131, 135-142, 183-188, 201-219, 227-233, 236-244, 246-252, 282-295, 307-315, 335-350, 392-399, 409-414, 427-433, 435-447, 468-482, 487-494, 500-506, 529-537, 543-550, 555-575, 577-584, 606-611, 619-625, 637-651, 682-711, 713-727, 729-738, 756-764, 783-795, 801-815, 817-830, 833-847, 861-890, 898-904, 909-928 and 406-428 of Seq ID No 358; 7-17, 36-53 and 41-65 of Seq ID No 359; 4-10, 17-24, 28-34, 42-49, 55-61, 70-106, 112-127, 135-142, 154-172, 178-184, 187-201, 213-219, 225-230, 235-246, 253-260, 267-302, 318-331, 339-353, 363-373, 376-387 and 258-268 of Seq ID No 360; 8-17, 29-43, 45-52, 58-69, 87-100, 102-111, 148-163, 172-187, 190-208, 210-227, 232-239, 245-253, 258-263, 286-299, 313-334, 346-362, 373-388, 391-411, 425-430, 434-446, 457-489, 496-502, 518-524, 537-546, 555-560, 602-610, 637-646, 676-689, 698-704, 706-742, 750-778, 780-791, 806-842, 864-879, 881-888, 890-899, 901-908, 910-921, 941-947, 953-959, 967-980, 990-995, 1000-1061, 1073-1079, 1081-1092, 1096-1118, 1121-1185, 1195-1209, 1219-1232, 1237-1243, 1250-1274, 1276-1282, 1302-1317, 1324-1333, 1339-1344, 1349-1361, 1370-1376, 1406-1413, 1415-1427, 1433-1450, 1453-1469, 1473-1478, 1482-1495, 1509-1517, 1519-1526 and 611-689 of Seq ID No 361; 7-29, 74-83, 101-107, 115-124, 127-142, 166-184, 209-215, 222-232, 245-252, 255-262 and 40-60 of Seq ID No 362; 4-44, 16-32, 42-47, 65-71, 82-109, 128-145, 158-171, 177-191, 197-228, 230-236 and 138-167 of Seq ID No 363; 4-22, 47-59, 61-71, 76-82, 96-103, 119-146, 165-172, 174-188, 191-202, 214-232, 240-247, 267-273, 278-293, 303-323, 326-340, 348-353, 365-387, 389-398, 405-411, 416-421, 423-451, 453-480, 482-495, 507-512, 518-529, 537-545, 563-570, 581-587, 591-597, 611-624, 626-634, 637-647, 671-692, 694-725, 735-742, 747-768, 786-825, 859-875, 887-894, 897-909, 915-938, 943-954, 967-976, 984-1001, 1006-1015 and 943-1016 of Seq ID No 364; 4-14, 24-34, 47-69, 81-90, 98-112, 144-153, 161-169, 189-196, 202-208, 213-220, 243-249, 256-262, 265-271, 279-285, 299-307, 310-324, 326-345, 356-369, 397-416, 424-429, 432-441 and 361-403 of Seq ID No 365; 4-22, 27-40, 44-54, 77-91, 112-127, 155-161, 196-207, 210-216, 228-234 and 171-210 of Seq ID No 366; 4-9, 14-23, 50-56, 59-68, 77-102, 111-120, 126-152, 161-167, 174-180, 189-202, 204-228, 237-245, 259-266, 278-285, 300-309 and 113-157 of Seq ID No 367; 6-26, 31-37, 41-47, 62-69, 71-93, 106-119, 126-136, 167-180, 196-202, 210-216, 242-254, 258-264, 272-286, 292-298, 300-309, 312-322, 346-352, 354-380, 385-392, 401-420, 434-449, 451-459, 465-473, 497-514, 555-562, 566-574, 604-612, 642-648, 657-669 and 517-643 of Seq ID No 368; 23-35, 71-77, 94-100, 134-140, 157-163, 185-191, 228-237, 255-265, 269-283, 310-315, 334-340, 366-392, 395-400, 404-411, 423-428, 434-445, 451-458, 468-478, 496-508, 512-519, 558-563, 565-582, 11-149 and 507-577 of Seq ID No 369; 14-20, 35-48, 53-63, 71-77, 95-101, 114-121, 123-130, 144-151, 153-160, 162-170, 187-197, 201-211 and 23-52 of Seq ID No 370; 7-17, 24-44, 63-70, 88-99 and 1-78 of Seq ID No 371; 21-39, 46-53, 68-96, 107-113, 118-124, 126-135, 158-185, 196-202, 204-213, 219-226, 246-253, 267-275, 277-285, 299-317, 319-338, 404-410, 421-428, 435-463, 92-170 and 178-199 of Seq ID No 372; 28-43, 47-55, 59-68, 72-79, 106-112, 121-139, 151-160, 168-175, 177-183, 194-212, 223-229, 232-248, 254-263, 270-276, 317-323, 331-338, 342-356, 363-369, 378-391, 415-424, 432-441, 443-456, 464-470, 499-505, 521-527, 534-552, 586-599, 624-634, 639-647, 651-667, 685-690, 694-702, 711-731, 733-744, 752-776, 784-791, 801-807, 837-859, 879-890, 906-914, 918-924, 926-940, 945-958, 965-971, 980-1002, 1010-1016, 1018-1028, 1034-1044, 1046-1053, 1065-1075, 1079-1092, 1095-1104; 1125-1142, 1154-1162, 1176-1181, 1194-1207, 1233-1244, 1252-1261, 1267-1274, 1283-1288, 1318-1324, 1327-1342 and 403-455 of Seq ID No 373; 17-25, 32-77, 82-91, 100-128, 163-169, 189-207, 211-218, 227-232, 239-245, 255-260, 278-300, 311-325, 342-356, 382-390, 393-401, 416-460, 467-487, 491-497, 505-512, 516-532, 551-565, 568-575, 594-601, 610-632, 638-643, 647-670, 672-685, 699-710, 712-726 and 290-399 of Seq ID No 374; 4-39, 56-73, 107-128, 134-142, 144-153, 155-183, 198-203, 205-212, 215-223, 232-244, 248-265, 273-292, 294-301, 304-311, 322-329, 338-343, 369-378, 397-404, 408-416, 420-426, 436-443 and 177-199 of Seq ID No 375; 4-22, 25-31, 35-41, 53-61, 74-83, 101-145, 157-162, 199-216, 247-257, 266-276, 282-289, 291-298, 306-313, 324-335, 345-353, 360-368, 392-400, 404-421, 432-445, 455-462, 478-486, 494-499, 501-511, 525-551, 554-561, 581-588, 600-613, 637-661, 669-676, 684-697, 699-705, 720-730, 746-751 and 393-543 of Seq ID No 376; 11-25, 65-79, 89-97, 106-112, 115-121, 126-132, 134-141, 218-230, 255-260, 287-294, 304-309, 328-334, 339-345, 347-363, 366-382, 421-428, 457-463, 471-477, 484-492, 504-511, 513-518, 547-554, 559-572, 598-604, 617-628, 641-647, 658-665, 691-696, 701-714, 744-751, 760-770, 774-780, 792-801, 805-817 and 635-686 of Seq ID No 377; 5-25, 31-39, 72-79, 93-102, 104-110, 122-132, 138-146, 157-189, 192-198, 205-214, 226-233, 240-248, 269-275, 282-298, 304-310, 313-327, 342-348 and 74-108 of Seq ID No 378; 7-34, 44-50, 54-64, 90-99, 101-106, 111-123, 156-175, 182-212, 224-232, 235-247, 249-264, 266-273, 306-321, 326-333, 343-351, 359-365, 370-403, 424-455, 466-477, 481-495, 503-511, 516-531, 534-543, 555-573, 578-600, 638-650, 657-671, 677-682, 686-692, 698-710, 721-729, 732-741, 752-763, 773-784, 786-809, 816-821, 829-849, 885-894, 911-920, 931-940, 942-949, 954-962, 979-986, 988-995, 1007-1016, 1034-1039, 1060-1065, 1076-1093, 1131-1137, 1144-1152, 1160-1165, 1170-1181, 1186-1196, 1220-1260, 1271-1280, 1287-1295, 1318-1328, 1346-1356, 1361-1367, 1378-1392, 1401-1407, 1412-1420, 1426-1443, 1478-1489, 1491-1499, 1501-1525 and 1352-1387 of Seq ID No 379; 18-53, 64-93, 95-105,124-135, 143-148, 155-161, 163-171, 184-198, 238-245, 258-271, 273-284, 287-292, 302-310, 312-320, 322-341, 349-365, 377-403, 407-414, 417-423, 444-453, 455-469, 471-495, 503-511, 536-557, 579-586, 588-609, 619-626, 632-638, 643-649, 656-663, 669-680, 682-688, 699-714, 729-739, 755-761, 768-776, 781-793, 801-815, 821-826, 833-842, 863-869 and 7-84 of Seq ID No 380; 8-15, 24-40, 51-65, 78-89, 102-111, 117-154, 164-177, 181-192, 198-209, 216-222, 230-237, 241-248, 254-268, 285-293, 298-321, 331-338, 366-373, 379-389, 392-415, 429-439, 441-451, 453-459, 471-486, 489-501, 524-535 and 1-26 of Seq ID No 381; 10-18, 26-38, 48-54, 60-69, 77-83, 88-95, 119-126, 133-169, 172-185, 193-206, 214-225, 236-250, 255-261, 269-275, 278-301, 320-329, 336-341, 345-353, 356-369, 389-397 and 64-88 of Seq ID No 382; 27-32, 37-50, 68-82, 84-108, 134-145, 147-154, 162-170, 172-182, 194-200, 205-224, 232-270, 293-299, 312-328 and 86-134 of Seq ID No 383; 7-13, 18-44, 64-74, 81-86, 94-104, 134-148, 153-159, 174-183, 204-225, 228-243, 248-255, 283-295, 297-303, 320-347 and 100-141 of Seq ID No 384; 4-27, 36-42, 55-62, 64-73, 92-106, 112-118, 120-127, 135-154, 170-179, 242-257, 270-277, 286-325, 335-341, 359-365, 381-387, 410-440, 470-478, 520-528, 543-553, 575-582, 603-612, 617-623, 628-649, 657-663, 685-691, 693-699, 703-708, 712-719, 740-747, 755-763, 766-782, 800-809, 811-833, 835-851, 856-862, 864-876 and 344-369 of Seq ID No 385; 4-10, 15-24, 26-53, 55-71, 78-83, 90-113, 128-148, 156-163, 165-179, 203-213, 228-239, 250-259, 277-285, 292-314, 322-330, 334-340, 345-360, 381-396, 404-409, 416-427 and 204-232 of Seq ID No 386; 4-17, 21-30, 42-49, 56-63, 67-73, 78-87, 92-99, 105-111, 122-130, 151-160, 168-197, 209-226, 243-276, 286-293, 295-301, 306-319, 322-332, 335-342 and 104-126 of Seq ID No 387; 4-10, 12-23, 28-34, 37-60, 65-84, 97-103, 113-127, 135-143, 182-187, 200-223, 227-233, 236-271, 274-279, 282-287, 293-299, 314-329, 334-358 and 300-362 of Seq ID No 388; 20-32, 37-46, 48-65, 75-83, 86-95, 121-133, 138-151, 183-190, 199-205, 216-227 and 1-38 of Seq ID No 389; 9-25, 29-48, 50-100, 102-126, 131-149, 167-173, 210-217, 224-256, 259-270, 275-292, 295-301, 308-313, 319-335, 337-359, 362-382, 393-423, 436-449, 468-476, 481-487, 492-500, 526-534, 537-548, 560-567, 569-579, 590-598, 604-613, 629-636, 644-656 and 506-577 of Seq ID No 390; 25-45, 53-78, 80-102, 116-128, 161-167, 180-186, 193-219, 235-258, 261-268, 291-318 and 214-233 of Seq ID No 391; 4-18, 25-31, 33-39, 47-53, 64-92, 97-106, 123-129, 134-146, 165-171, 173-190, 192-213, 226-239, 251-273, 283-298, 316-324, 339-345, 350-356, 361-376, 400-408, 418-440, 444-451, 476-481, 505-516, 524-542, 555-563, 581-594, 607-629, 634-641, 647-670, 711-719, 728-738, 755-765, 772-780, 800-815, 822-833, 842-852, 860-865, 874-880, 891-913, 926-938, 941-946, 961-978, 984-990, 1013-1024, 1052-1092, 1099-1111, 1120-1140, 1153-1168, 1170-1190, 1193-1211, 1221-1233, 1253-1264, 1268-1274, 1283-1289, 1295-1300, 1303-1327, 1338-1351, 1362-1368, 1391-1396, 1403-1416, 1429-1436, 1471-1477, 1483-1513, 1526-1555, 1585-1591, 1596-1630, 1632-1639 and 299-326 of Seq ID No 392; 10-25, 34-54, 57-67, 77-96, 111-121, 127-139, 151-157, 161-179, 183-198, 201-219, 233-239, 247-252, 268-276, 283-294, 299-309, 319-324 and 156-268 of Seq ID No 393; 6-28, 34-45, 64-79, 88-95, 98-115, 120-141, 159-167, 174-179, 186-192, 198-208, 216-225, 232-246, 248-273, 275-283, 291-299, 304-316, 370-381, 386-393, 401-417, 421-445, 460-468, 470-487, 497-509, 511-535, 542-558, 564-574, 603-609, 619-648, 661-675, 682-694, 720-738, 742-759, 762-788, 793-805, 825-851, 885-893, 898-906, 918-935, 941-953, 971-978, 986-993, 1001-1017, 1019-1026, 1050-1070, 1072-1089, 1097-1102, 1107-1121 and 934-1003 of Seq ID No 394; 6-13, 31-38, 47-60, 71-102, 107-124, 128-155, 173-180, 213-220 and 202-243 of Seq ID No 395; 4-38,49-71, 75-85, 110-115, 168-173, 202-210, 221-228, 240-245, 258-264, 302-316, 348-362, 386-391, 456-462, 474483, -494-499, 511-516, 523-528, 533-539, 549-557, 579-585, 587-593, 618-625, 627-634, 654-660, 664-670, 682-688, 697-702, 729-735, 783-793, 804-812, 817-829, 862-868, 908-920, 954-960, 1000-1006, 1008-1031, 1044-1050, 1069-1077, 1079-1084, 1097-1118, 1139-1146, 1152-1158, 1165-1176, 1181-1186, 1201-1213, 1261-1267, 1272-1280, 1282-1289, 1358-1364, 1373-1382, 1390-1400, 1443-1450, 1497-1505, 1530-1552, 1560-1568, 454-483 and 1142-1349 of Seq ID No 396; 4-13, 20-34, 47-53, 58-65, 76-82, 89-106, 139-160, 165-182, 191-205 and 10-41 of Seq ID No 397; 31-42, 59-75, 91-102, 104-123, 147-153, 172-184, 193-206, 257-266, 306-316, 318-329 and 4-34 of Seq ID No 398; 5-14, 26-35, 38-45, 54-60, 63-79, 121-127, 137-145, 152-162, 167-173, 175-183, 191-202, 218-228, 238-263, 278-295, 303-316, 320-335, 337-345, 359-365, 382-400 and 64-148 of Seq ID No 399; 4-17, 31-39, 46-61, 68-73, 76-97, 128-139, 150-156, 166-172, 174-182, 184-215, 219-225, 238-245, 249-262 and 187-223 of Seq ID No 400; 4-23, 30-41, 44-53, 58-70, 82-91, 107-114, 122-129, 148-155, 201-207, 223-232 and 1-69 of Seq ID No 401; 4-16, 28-41, 44-52, 60-66, 73-82, 92-101, 108-114, 133-138, 145-155, 177-185, 194-202 and 89-130 of Seq ID No 402; 4-9, 21-39, 72-78, 82-88, 99-131, 136-143, 151-162, 164-187, 189-204, 208-216, 223-229, 232-240, 246-256, 269-283, 288-299, 311-321, 328-335 and 209-237 of Seq ID No 403; 4-14, 36-48, 66-73, 75-89, 95-103, 115-123, 128-133, 140-145, 151-158, 165-176, 178-188, 224-254, 267-278, 289-297, 302-311 and 178-262 of Seq ID No 404; 19-25, 55-70, 76-82, 88-107, 114-129, 136-145, 154-177, 205-219, 227-233 and 1-35 of Seq ID No 405; 26-33, 39-45, 50-62, 76-85, 87-101, 116-131, 142-152, 154-186, 193-199, 201-217, 221-243, 266-272, 281-298, 324-330, 335-342, 345-355, 375-383, 407-413, 254-315 and 323-407 of Seq ID No 406; 4-22,27-36, 60-69, 90-98, 107-113, 117-123, 127-134, 137-151, 154-161, 169-178, 185-192, 202-208, 214-223, 230-239, 245-255, 266-275, 307-317, 323-337, 339-353, 361-379, 385-391, 393-401, 415-422, 424-429, 434-442, 444-449, 470-480 and 358-400 of Seq ID No 407; 4-25, 31-42, 83-101, 109-123, 127-136, 139-145, 154-166, 169-182, 194-201, 210-220, 226-237, 251-275, 277-304, 309-329, 341-362, 367-372, 377-393, 400-406 and 223-239 of Seq ID No 408; 4-22, 29-34, 37-44, 48-78, 98-110, 127-142, 144-156, 158-165 and 59-127 of Seq ID No 409; 4-12, 14-20, 27-34, 39-47, 51-67, 69-81, 89-97, 105-119, 121-133, 140-149, 151-161 and 67-105 of Seq ID No 410; 6-19, 25-35, 43-48, 56-69, 73-93, 137-146, 152-161, 164-207, 212-229, 236-241, 244-250, 273-288, 292-299, 314-324 and 259-291 of Seq ID No 411; 17-24, 34-40, 78-85, 227-233, 294-315, 327-335, 345-351, 354-359, 363-368, 388-403, 405-411, 413-419, 425-434, 462-472, 480-500, 528-536, 542-560, 566-573, 579-589, 593-606, 614-646, 651-658, 663-669, 686-726, 734-747, 754-778, 787-806, 809-825, 827-839, 876-887 and 80-214 of Seq ID No 412; 4-9, 15-29, 38-43, 50-81, 83-96, 98-108, 116-122, 136-143 and 133-148 of Seq ID No 413; 5-79, 98-105, 133-146, 158-182, 189-207, 213-225, 231-252, 272-278, 283-303, 312-317, 333-346, 361-367, 370-379, 387-419, 421-433, 439-453, 460-468 and 132-153 of Seq ID No 414; 9-29, 35-40, 49-63, 69-76, 110-134, 141-147, 160-169 and 63-101 of Seq ID No 415; 4-9, 13-20, 25-43, 50-56, 75-86, 102-115, 120-126, 128-135, 139-145, 161-166, 170-189, 202-210, 212-219, 221-232, 240-248, 252-264, 54-161 and 256-285 of Seq ID No 416; 7-17, 19-33, 44-51, 56-70, 74-79, 85-93, 96-102, 110-117, 124-132, 140-148, 157-176, 204-248, 256-269, 289-306, 318-324, 331-339, 377-382, 389-397, 399-411, 413-420, 424-432, 436-441, 462-469, 499-506, 522-547, 549-563, 569-575, 578-594, 609-614, 621-630, 637-646, 652-685, 688-711, 713-724, 739-744, 752-783, 793-799, 811-823, 825-841, 846-855, 861-868, 874-886, 895-907, 935-966, 977-1024, 1026-1035, 1037-1055, 1063-1091, 1094-1103, 1105-1119, 1128-1149, 1160-1173, 1182-1194, 1210-1222, 1227-1234, 1244-1258, 1275-1285, 1293-1300, 1316-1322, 1336-1354, 1357-1364, 1367-1372, 1386-1392, 1403-1411, 1424-1430, 1439-1456, 1458-1469, 1485-1504, 1018-1052 and 1134-1262 of Seq ID No 417; 4-30, 32-42, 59-65, 78-88, 104-127, 132-147, 158-171, 181-187, 195-214, 220-226, 238-269 and 6-90 of Seq ID No 418; 10-16, 25-53, 64-74 and 1-83 of Seq ID No 419; 4-29, 48-57, 75-86, 99-113, 146-155, 164-174, 190-201, 215-234, 236-251 and 101-185 of Seq ID No 420; 4-9, 32-56, 58-67, 71-81, 90-95, 97-105, 112-118, 124-132, 138-144, 147-167, 170-177, 211-217, 231-241, 250-258, 260-272, 274-282, 289-296, 299-309, 319-331, 344-350, 356-362, 368-377, 381-394, 399-406, 412-430, 432-450, 459-473, 486-503, 508-515, 520-548, 564-570, 581-587, 616-623, 628-635, 638-660, 678-684, 691-696, 703-709, 716-723, 760-772, 787-795, 835-844 and 177-207 of Seq ID No 421; 5-43, 46-81, 88-95, 137-142, 163-191, 195-203, 210-235, 241-254, 256-276, 280-288, 292-305, 307-313, 317-333, 335-343, 347-353, 357-363, 372-381, 384-389, 399-409 and 58-179 of Seq ID No 422; 26-32, 38-44, 68-75, 85-100, 104-114, 126-132, 140-150, 153-164, 175-193, 200-209, 218-224, 226-232, 243-249, 251-260, 275-293, 304-329, 335-353, 364-479, 485-490, 500-512, 514-523, 532-556, 577-589, 622-628, 631-653, 656-678, 542-627 and 691-724 of Seq ID No 423; 8-22, 25-30, 46-62, 67-73, 98-103, 105-114, 120-141, 144-153, 168-175, 181-193, 198-204, 208-227, 235-242, 249-258, 281-288, 291-306, 327-336, 340-361, 368-380, 389-409, 417-426, 428-435, 442-453, 468-486, 488-496, 498-509, 511-523, 540-553, 566-579, 587-603, 629-636, 677-682 and 170-207 of Seq ID No 424; 9-25, 41-61, 68-75, 81-102, 106-141, 158-165, 173-191 and 32-53 of Seq ID No 425; 7-26, 28-37, 43-58, 67-79, 92-99, 103-111, 118-128, 130-139, 152-165, 170-186, 192-214, 216-223, 225-251 and 95-122 of Seq ID No 426; 4-16, 21-36, 38-47, 54-64, 92-103, 117-126, 134-155, 157-200, 202-223, 246-262and 70-163 of Seq ID No 427; 30-38, 45-51, 75-90, 93-114, 119-127, 134-143, 151-159, 166-180, 189-207, 217-222, 230-239, 267-276, 283-292, 324-350, 374-386, 392-403, 409-416, 418-424, 451-458, 466-477, 483-494, 501-509, 521-528, 540-549, 556-561, 573-579, 581-588, 621-626, 628-636, 654-661, 695-701, 711-717, 734-743, 751-757, 764-771, 789-798, 832-837, 860-867, 869-883, 911-917, 942-950, 958-964, 966-981, 992-999, 44-131 and 484-615 of Seq ID No 428; 5-14, 27-42, 48-67, 71-83, 85-91, 105-112, 114-135, 139-147, 159-165, 169-185, 188-195, 199-208, 212-221, 231-253, 264-272, 275-282, 290-303, 309-319, 324-331, 340-358, 380-405, 419-425, 438-444, 450-463, 468-477, 497-514, 520-533, 549-556, 568-574, 617-626, 637-643, 661-668, 674-684, 705-713, 718-733, 735-775 and 541-569 of Seq ID No 429; 13-19, 21-32, 45-69, 79-87, 90-109, 140-148, 156-208, 215-232, 243-274, 276-284, 288-298, 301-316, 339-347, 369-384, 405-412, 430-437, 445-457, 464-470, 475-483, 490-509, 517-524, 532-591, 609-628, 647-677, 681-709, 731-740, 752-767, 770-781, 787-793, 798-807, 825-836, 839-869 and 104-137 of Seq ID No 430; 4-10, 33-44, 73-78, 93-101, 123-129, 135-165, 201-214, 251-261, 268-274, 285-292, 316-322, 328-336, 342-350, 353-360, 374-387, 391-399, 401-406, 417-425, 437-443, 447-454, 511-522, 530-535, 727-737, 762-774, 781-787, 803-809, 827-838, 852-859, 877-883, 901-907, 910-918, 951-956, 996-1002, 1071-1079, 1086-1096, 1098-1104, 1106-1119, 1129-1135, 1142-1148, 1155-1161, 1167-1185, 1199-1205, 1224-1232, 1242-1251, 1279-1287, 1293-1299, 1305-1321, 1372-1396, 1426-1435, 1467-1473, 1479-1492, 1526-1533, 1548-1558, 1578-1585 and 1520-1553 of Seq ID No 431; 4-10, 36-45, 56-92, 108-114, 125-133, 137-146, 156-162, 164-186, 194-203, 225-234, 242-251, 272-283, 285-306, 310-315, 322-330, 358-371, 373-379, 389-396 and 95-189 of Seq ID No 432; 4-32, 38-46, 66-83, 88-95, 110-118, 123-141, 169-180, 200-208, 217-225, 237-245, 247-261, 263-272, 275-282, 291-302, 310-338, 345-353, 360-369, 371-378, 386-394, 398-413, 416-422, 437-448 and 51-81 of Seq ID No 433; 4-10, 12-38, 59-64, 81-97, 122-132, 136-142, 149-165, 180-187, 192-199, 205-216, 222-228, 244-251, 255-274, 280-286, 294-320, 327-333, 339-346, 353-359, 372-385, 402-408, 413-420, 433-440, 443-453, 456-461, 464-472, 483-495 and 14-27 of Seq ID No 434; 4-40, 42-56, 59-73, 76-110, 115-128, 132-139, 148-170, 174-195, 197-207, 214-220, 222-236, 238-246, 252-283, 291-326, 334-413 and 239-261 of Seq ID No 435; 4-11, 18-26, 31-47, 59-68, 74-92, 98-144, 149-158, 173-180, 200-210, 216-223, 239-250 and 18-66 of Seq ID No 436; 4-30, 42-52, 59-67, 70-76, 80-86, 138-147, 154-159, 206-217, 26-232, 240-248, 250-257, 259-265, 280-294, 299-326, 328-334, 340-355, 393-398, 415-422, 440-447, 452-458 and 375-452 of Seq ID No 437; 4-12, 20-31, 43-49, 100-118, 121-138, 141-148, 153-161, 167-177, 206-213, 225-231, 235-240, 256-267, 277-287, 301-322, 325-333, 336-360, 381-388, 400-415, 447-452, 459-472 and 55-75 of Seq ID No 438; 4-10, 29-56, 93-99, 119-124, 133-140, 159-171, 187-195, 200-214, 221-232, 249-255, 263-271, 285-291, 310-316 and 61-198 of Seq ID No 439; 9-15, 48-65, 72-79, 87-102, 104-115, 118-124, 126-138, 153-185, 188-207, 212-239, 257-265, 297-304, 306-313 and 1-54 of Seq ID No 440; 18-38, 48-62, 68-107, 143-158, 167-181, 193-198, 205-213, 220-231, 239-245, 258-264, 279-300, 308-314, 318-328, 343-354, 360-367, 425-433, 465-474, 496-505, 508-514, 529-536, 545-562, 572-580, 587-593, 595-609, 612-618, 627-637, 642-649, 652-673, 688-693, 696-701, 707-736, 748-754, 766-776, 779-786, 791-797, 815-825, 830-842, 857-865, 868-876, 880-887, 898-905, 911-923, 925-936, 961-983, 1011-1018, 1043-1059, 1073-1079, 1093-1104, 1110-1116, 1135-1144, 1146-1163, 1183-1189, 1196-1204, 1222-1242, 1250-1262, 1275-1296, 1322-1330 and 1056-1199 of Seq ID No 441; 15-27, 35-40, 47-55, 57-73, 77-93, 103-112, 126-138, 141-179, 192-218, 224-237, 244-257, 263-278 and 83-115 of Seq ID No 442; 4-22, 24-29, 36-43, 63-75, 90-96, 118-128, 137-145, 168-182, 198-210, 212-221, 242-250, 289-316, 318-323, 327-339, 381-387, 401-411, 424-434, 443-449, 453-465, 485-498, 500-508, 510-515, 521-528, 538-545, 554-560, 574-606, 619-627, 645-658, 681-688, 70-79 and 473-516 of Seq ID No 443; 8-18, 45-50, 52-62, 76-82, 84-107, 109-116, 130-137, 141-150, 152-158, 164-170, 175-186, 188-196 and 9-73 of Seq ID No 444; 4-24, 39-46, 84-95, 133-151, 166-174, 179-189, 196-203, 212-218, 223-236, 240-246, 255-261, 266-275, 286-293, 299-316, 323-331, 347-358, 368-380, 382-389, 391-398, 410-417, 420-429, 437-445, 451-456, 464-471, 504-514, 523-532, 539-545, 553-567, 572-588, 594-603, 620-630, 636-641, 656-663, 665-672, 676-687, 693-700, 40-166 and 577-605 of Seq ID No 445; 442, 48-59, 74-90, 92-119, 121-149, 163-180, 185-192, 199-209 and 1-9 of Seq ID No 446; 5-26, 60-76, 104-114, 119-128, 136-141, 156-167, 186-198, 218-237, 260-267, 275-290, 328-335 and 294-334 of Seq ID No 447; 4-10, 14-37, 40-47, 68-77, 87-95, 103-111, 116-153, 170-237, 245-251, 253-274, 280-299, 311-318, 321-338, 364-371, 378-392, 395-430, 438-451, 458-475, 479-507, 520-526, 542-560, 573-586, 591-598, 608-614, 636-668, 678-690, 692-698, 702-717, 724-731 and 302-448 of Seq ID No 448; 5-17, 23-48, 60-73, 75-82, 98-108, 110-128, 146-160, 168-180, 191-213, 229-237, 240-252, 269-277, 305-313 and 63-147 of Seq ID No 449; 4-10, 19-35, 41-47, 51-60, 70-80, 91-115, 170-191, 194-211, 226-232, 234-241, 256-273, 289-294, 311-349, 358-363, 392-400, 406-416 and 356-391 of Seq ID No 450; 5-25, 50-57, 67-75, 78-86, 94-112, 122-145, 152-165, 171-183, 193-199, 217-235, 238-253, 255-264, 281-287, 294-303, 309-314, 319-324, 327-341, 349-355, 364-382, 384-392, 397-411, 419-427, 435-452, 455-463, 488-504, 536-541, 558-563, 568-574, 595-601, 614-620, 637-644 and 7-127 of Seq ID No 451; 10-16, 39-45, 62-91, 102-114, 120-127, 136-147, 152-159, 163-173, 178-188, 196-217, 223-231, 234-254, 257-267, 270-283, 290-300, 306-312 and 224-295 of Seq ID No 452; 4-9, 12-21, 23-52, 54-65, 74-83, 103-117, 131-141, 144-163, 171-177, 183-189, 205-212, 252-262, 297-304, 308-314, 320-329, 343-349, 357-370, 375-380, 395-405, 434-441, 456-465, 474-484, 505-514, 528-536, 540-549, 576-582, 597-607, 616-622, 634-641, 648-654, 690-713, 715-724, 743-751, 757-763, 772-789, 791-802, 809-814, 828-837, 840-846, 853-875 and 283-379 of Seq ID No 453; 4-27, 40-46, 55-62, 84-100, 108-114, 118-123, 132-145, 165-171, 178-183, 192-223, 226-232, 234-243, 276-282, 299-305, 326-334, 340-351, 357-371, 374-387, 395-406, 417-437, 443-452, 470-478, 485-494, 496-503, 508-521, 527-537, 541-546 and 252-272 of Seq ID No 454; 4-36, 45-50, 58-63, 69-80, 89-97, 99-109, 111-118, 126-132, 141-147, 172-184, 188-197, 208-215, 220-231, 236-241, 253-264, 271-280, 288-297, 342-347, 361-367, 375-382, 388-394, 401-406, 408-414, 441-447, 452-458, 466-476, 483-491, 503-510, 521-528, 539-545, 547-558, 566-576, 584-589, 606-617, 624-636 and 418-432 of Seq ID No 455; 7-14, 5-32, 6-72, 95-100, 108-114, 123-135, 143-153, 203-221, 224-230, 260-269, 290-297, 302-308, 320-328, 333-339 and 149-248 of Seq ID No 456; 21-27, 30-48, 55-65, 70-90, 97-107, 122-128, 135-166, 172-180, 184-199, 205-224, 237-247, 252-269, 278-283 and 240-257 of Seq ID No 457; 4-14, 20-33, 36-43, 49-60, 72-114, 117-123, 125-132, 138-143, 157-175, 184-204, 208-217 and 58-89 of Seq ID No 458; 4-15, 23-36, 38-47, 54-64, 92-103, 117-126, 135-155, 157-200, 202-223, 231-239, 246-261 and 70-162 of Seq ID No 459; 4-10, 35-45, 56-92, 108-114, 127-146, 160-186, 194-203, 225-234, 242-251, 272-283, 285-306, 310-315, 322-330, 358-371, 373-382, 389-396 and 93-188 of Seq ID No 460; 18-32, 35-82, 85-115, 119-142, 149-172 and 4-36 of Seq ID No 461; 6-14, 20-28, 35-55, 64-87, 100-109, 144-149, 189-208, 210-218, 221-227, 242-247, 254-264, 283-297, 301-308, 310-322, 351-358, 372-378, 383-389, 421-432, 447-460, 537-545, 550-558, 581-593, 595-606, 645-658, 677-688 and 414-503 of Seq ID No 462; 9-34, 47-63 and 37-51 of Seq ID No 463; 6-22, 50-58, 73-101, 119-128, 139-154, 167-173, 209-217, 227-234 and 1-126 of Seq ID No 464; 10-34, 37-44, 72-78, 87-100, 111-117, 122-143, 177-196, 207-229, 232-249, 255-261, 268-278, 289-300, 315-349, 351-358, 371-378, 386-394, 404-466, 468-480 and 341-443 of Seq ID No 465; 16-22, 30-35, 45-52, 54-64, 74-84, 90-98, 120-125, 135-148, 156-162, 166-186, 190-199, 201-215, 226-232, 262-270, 281-289, 322-331, 335-340, 367-372, 379-385, 415-426, 438-445, 455-466, 472-479, 492-497 and 341-484 of Seq ID No 466; 4-31, 33-66, 77-83, 90-101, 118-135, 161-166, 168-189, 203-209, 216-222, 231-236, 269-277, 279-290, 298-310, 341-347, 371-376, 380-387, 389-395, 410-419, 446-455, 465-471, 476-484, 513-522, 532-537, 545-556, 560-585, 603-610, 630-657, 660-672, 694-704, 717-728, 738-745, 754-766, 782-792, 794-811, 813-819, 833-853, 901-906, 912-917, 951-979, 985-991, 998-1004, 1013-1019, 1052-1065, 1080-1086, 1124-1130, 1142-1150, 1168-1176, 1182-1193, 1209-1218, 1234-1245, 1271-1277, 1284-1298, 1301-1308, 1339-1345 and 529-629 of Seq ID No 467; 21-28, 57-71 and 1546 of Seq ID No 468; 63-99, 101-109, 111-137, 143-172, 175-200 and 11-48 of Seq ID No 469; 18-32, 35-82, 85-115, 119-142, 149-172 and 6-39 of Seq ID No 470; 67-74, 88-94, 112-118, 127-138, 155-169, 171-180, 183-190, 196-205, 243-249, 260-271, 308-344, 346-373, 381-414, 416-457, 473-513, 515-524, 528-535, 539-544, 556-566, 572-580, 585-590 and 26-129 of Seq ID No 471; 17-40, 47-66, 70-78 and 29-102 of Seq ID No 472; 4-9, 23-41, 44-51, 58-64, 70-86, 94-111 and 47-77 of Seq ID No 473; 4-22, 29-48, 50-58, 62-69, 71-78 and 6-35 of Seq ID No 474; 8-19, 31-47, 49-58, 64-79, 84-96 and 7-52 of Seq ID No 475; 4-17, 19-26, 41-49, 63-87, 92-99, 113-131 and 9-118 of Seq ID No 476; 10-37, 55-68, 71-78, 92-98, 115-122, 131-138, 149-158, 163-170, 172-189, 212-219, 239-257, 259-271, 289-302, 304-320, 322-340, 359-366, 373-384, 400-412, 444-453, 460-474, 485-527 and 186-224 of Seq ID No 477; 6-13 of Seq ID No 478; 5-12 of Seq ID No 480; 6-14 of Seq ID No 481; 4-10 of Seq ID No 483; 4-19 of Seq ID No 486; 9-14 of Seq ID No 487; 4-44, 47-60, 65-72, 92-98 and 61-95 of Seq ID No 488; 2-17 of Seq ID No 489; 19-29, 39-45, 52-59 and 7-32 of Seq ID No 490; 6-20, 36-44 and 78-97 of Seq ID No 491; 5-12 and 1-22 of Seq ID No 492; 17-24, 2641, 50-55 and 8-32 of Seq ID No 493; 7-18 and 13-22 of Seq ID No 494; 24-32 and 3-28 of Seq ID No 495; 6-11, 14-35, 40-56 and 17-35 of Seq ID No 496; 4-12, 17-23, 42-53, 69-81 and 45-64 of Seq ID No 497; 4-25, 28-34, 37-43, 59-69, 104-114 and 52-117 of Seq ID No 498; 4-12 and 4-22 of Seq ID No 499; 4-9 and 1-19 of Seq ID No 500; 4-22 and 2-19 of Seq ID No 501; 13-36, 48-63, 80-101, 141-149, 165-176, 184-198 and 28-54 of Seq ID No 502; 4-14, 21-26 and 21-29 of Seq ID No 503; 4-26 and 20-36 of Seq ID No 504; 4-13 and 7-22 of Seq ID No 505; 7-13, 21-34 and 26-54 of Seq ID No 506; 4-21, 25-44, 59-68 and 1-92 of Seq ID No 507; 4-14, 26-33 and 12-33 of Seq ID No 508; 12-31, 46-60, 87-94 and 1-98 of Seq ID No 509; 5-14 and 4-22 of Seq ID No 510; 6-15, 34-52 and 14-36 of Seq ID No 511; 5-18, 27-33, 40-48 and 17-39 of Seq ID No 512; 8-17, 63-70, 89-100 and 21-52 of Seq ID No 513; 4-18, 21-31, 51-68 and 34-66 of Seq ID No 514; 4-9, 14-19 and 3-31 of Seq ID No 515; 4-10, 21-30, 37-51 and 11-44 of Seq ID No 516; 4-9, 32-38 and 7-22 of Seq ID No 517; 4-22, 29-36, 38-47 and 12-43 of Seq ID No 518; 4-12, 14-23 and 26-37 of Seq ID No 519; 4-11, 20-46 and 32-59 of Seq ID No 520; 4-10 and 1-30 of Seq ID No 521; 4-28, 32-50 and 17-51 of Seq ID No 522; 14-28 and 2-14 of Seq ID No 523; 4-25 and 12-40 of Seq ID No 524; 33-41, 43-53 and 16-39 of Seq ID No 525; 9-16 and 7-18 of Seq ID No 526; 4-11, 24-31 and 18-34 of Seq ID No 527; 14-20, 48-56, 62-69, 81-87 and 456 of Seq ID No 528; 8-16 and 13-39 of Seq ID No 529; 11-16, 26-40, 50-63 and 6-48 of Seq ID No 530; 23-34, 59-72 and 1-28 of Seq ID No 531; 11-16, 26-82, 95-101, 103-113, 120-164, 179-185, 187-194, 224-248, 255-263, 276-293, 296-301, 304-312, 314-320, 351-374, 390-398, 401-407, 420-426, 434-444, 454-475, 481-508, 521-531, 535-549 and 46-77 of Seq ID No 532; 6-30, 32-53, 69-76, 78-86, 96-112, 121-135, 142-175, 177-199, 201-255, 263-269, 277-288, 290-303, 307-345, 353-364, 388-403, 443-455, 462-474, 480-485 and 6-30 of Seq ID No 533; 12-61 and 14-46 of Seq ID No 534; 4-12, 24-33, 39-51, 57-63, 78-87 and 26-51 of Seq ID No 535; 8-15, 29-40, 48-54 and 33-56 of Seq ID No 536; 22-31, 59-69 and 70-100 of Seq ID No 537; 12-61 and 12-43 of Seq ID No 538; 4-16, 28-39, 62-71, 85-97 and 54-85 of Seq ID No 539; 10-20, 23-31, 35-42, 48-62 and 30-53 of Seq ID No 540; 9-41, 46-52, 70-85 and 63-89 of Seq ID No 541; 18-34, 36-46, 71-83, 95-101, 130-143, 149-161 and 96-115 of Seq ID No 542; 4-18, 26-66, 68-95, 100-110, 120-135, 143-165, 168-175, 177-198 and 160-176 of Seq ID No 543; 4-16, 21-34, 51-56 and 10-29 of Seq ID No 544; 4-23, 29-37, 40-70, 78-91, 98-111 and 89-115 of Seq ID No 545; 4-34, 56-62 and 36-54 of Seq ID No 546; 4-9 and 1-29 of Seq ID No 547; 10-22, 24-37, 52-57, 74-81 and 16-69 of Seq ID No 548; 4-16, 42-70, 78-93, 95-101, 103-111 and 64-88 of Seq ID No 549; 4-9, 22-48, 51-59, 64-94, 100-124, 130-135 and 123-134 of Seq ID No 550; 10-20, 27-40, 48-57 and 15-58 of Seq ID No 551; 24-37 and 12-30 of Seq ID No 552; 30-42, 51-56, 67-76, 79-96 and 22-61 of Seq ID No 553; 11-22, 28-35 and 18-33 of Seq ID No 554; 4-9, 13-21, 37-42 and 23-36 of Seq ID No 555; 4-12 and 12-22 of Seq ID No 556; 39-50, 71-89, 95-106, 126-139, 154-162 and 58-143 of Seq ID No 557; 5-26, 38-46, 54-62, 69-81, 87-99, 103-117, 120-136, 138-161, 168-189, 201-207 and 136-167 of Seq ID No 558; 4-21, 33-39, 41-55, 61-68, 73-98, 104-110, 121-127, 131-156 and 97-115 of Seq ID No 559; 9-26, 36-48 and 21-40 of Seq ID No 560; 4-12, 26-32 and 12-24 of Seq ID No 561; 4-12, 17-23, 39-62 and 8-34 of Seq ID No 562; 17-41, 47-66 and 12-30 of Seq ID No 563; 4-11, 15-25, 33-52 and 1-27 of Seq ID No 564; 4-11, 33-40, 48-76, 96-104 and 84-106 of Seq ID No 565; 15-22 and 11-26 of Seq ID No 566; 8-19, 44-53, 61-71, 78-85, 97-107 and 49-89 of Seq ID No 567; 13-23, 31-44, 59-66, 84-90, 96-110 and 47-147 of Seq ID No 568; 4-24, 56-73, 83-97, 112-132, 140-150, 161-184 and 111-146 of Seq ID No 569; 4-17, 19-26, 41-48, 63-87, 92-99, 113-131 and 10-136 of Seq ID No 570; 4-11, 17-26 and 6-32 of Seq ID No 571; 26-38, 48-55, 72-86, 90-107, 123-132, 134-161, 181-187 and 3-19 of Seq ID No 572; 4-19, 26-35 and 11-46 of Seq ID No 573; 5-10, 21-38, 42-70, 84-103 and 92-127 of Seq ID No 574; 6-17, 34-40, 42-51, 75-85, 94-100 and 38-63 of Seq ID No 575; 6-13, 21-27, 29-35, 57-64 and 32-63 of Seq ID No 576; 4-19, 22-48, 54-62, 73-91, 94-109 and 75-98 of Seq ID No 577; 9-25 and 12-28 of Seq ID No 578; 16-21 and 8-25 of Seq ID No 579; 4-24, 26-32, 45-55, 58-67, 76-93, 104-111, 117-122, 128-136 and 33-119 of Seq ID No 580; -11, 31-37, 56-63, 66-84 and 32-58 of Seq ID No 581; 6-69, 75-87, 89-111, 149-156 and 52-139 of Seq ID No 582; 4-13, 19-29, 49-68 and 24-49of Seq ID No 583; 19-25, 27-33, 43-84, 86-92, 111-118, 125-136, 138-147 and 95-124 of Seq ID No 584; 20-29, 50-56, 63-85, 89-98, 110-128 and 2-29 of Seq ID No 585; 4-11, 41-47, 62-71 and 7-34 of Seq ID No 586; 23-30, 48-57, 67-72, 81-89 and 11-31 of Seq ID No 587; 14-27, 50-62 and 22-57 of Seq ID No 588; 4-17, 23-37 and 13-33 of Seq ID No 589; 5-33, 38-57, 60-68 and 18-37 of Seq ID No 590; 4-19, 44-51 and 24-45 of Seq ID No 591; 17-40, 47-66, 70-78 and 67-101 of Seq ID No 592; 4-20, 24-29, 43-55, 57-63, 74-83, 137-157, 171-179, 181-192, 237-243, 273-279, 300-310, 312-321, 324-330, 366-380, 401-417 and 61-120 of Seq ID No 593; 6-21 and 17-43 of Seq ID No 594; 11-17 and 9-31 of Seq ID No 595; 18-28, 38-44, 53-59, 64-74, 78-85 and 10-96 of Seq ID No 596; 12-40, 49-58 and 5-49 of Seq ID No 597; 28-34, 46-52, 54-68, 72-85, 95-104 and 66-94 of Seq ID No 598; 4-14 and 1-25 of Seq ID No 599; 418, 30-37, 51-65, 83-89, 99-105, 108-136 and 24-98 of Seq ID No 600; 26-34, 36-44, 68-78, 85-92, 96-101, 127-134, 141-148, 156-166, 186-192, 244-256, 281-287, 291-301, 308-316, 321-343, 368-382, 385-391, 394-404, 414-429, 453-465, 471-489 and 147-240 of Seq ID No 997; 10-18, 26-48, 58-68, 72-78, 94-105, 115-130, 155-164, 170-177, 179-185, 201-219, 243-260, 267-277, 295-302, 350-376, 398-403, 429-437, 451-462, 471-478, 504-512, 563-568, 570-583, 589-594, 614-629, 1-123 and 161-546 of Seq ID No 998; 10-20, 35-45, 59-93, 163-168, 190-196, 200-210, 233-261, 270-279, 306-329, 331-353, 363-381, 388-394, 399-425, 443-460, 462-474, 477-498, 506-516, 522-542, 546-559, 570-577, 100-301 and 446-518 of Seq ID No 999; 4-13, 28-34, 52-71, 78-90, 118-140, 147-156, 167-187, 264-275, 286-292, 305-310, 328-334, 340-346, 351-362, 31-117 and 165-354 of Seq ID No 1000; 38-45, 76-84, 91-103, 111-118, 147-162, 166-177, 187-201, 208-215, 242-249, 267-274, 295-301, 309-322, 18-100 and 111-296 of Seq ID No 1001; 4-9, 25-39, 41-47, 70-78, 82-103, 106-140, 152-188, 192-198, 200-207, 211-217, 232-251, 271-277, 285-299, 307-314, 323-335, 344-370, 376-382, 388-398, 417-422, 428-446, 448-456, 462-468, 494-504, 526-533, 565-573, 199-383 and 454-579 of Seq ID No 1002; 10-27, 50-87, 94-109, 115-128, 134-152, 155-169, 175-193, 196-213, 216-233, 235-251, 259-270, 272-283, 293-303, 311-318, 327-339, 348-374, 380-386, 392-402, 421-426, 432-450, 452-460, 466-472, 498-508, 530-537 and 206-363 of Seq ID No 1003; 9-16, 25-31, 48-86, 90-101, 109-127, 131-147, 150-187, 189-195, 204-211, 213-226, 237-248, 250-261, 271-281, 289-296, 305-317, 326-352, 358-364, 370-380, 399-404, 410-428, 430-438, 444-450, 476-486, 508-515, 547-555, 1-118, 208-283 and 494-564 of Seq ID No 1004; 31-37, 57-66, 73-97, 99-117, 119-137, 141-149, 157-172, 179-185, 190-196, 203-212, 216-224, 226-238, 240-251, 261-271, 279-286, 295-307, 316-342, 348-354, 360-370, 389-394, 400-418, 420-428, 434-440, 466-476, 498-505, 537-545, 1-98, 233-351 and 423-538 of Seq ID No 1005; 27-37, 44-58, 66-80 and 5-70 of Seq ID No 1006; 4-32, 35-69, 92-98, 113-137, 168-190, 224-235, 269-277, 279-289, 322-329, 370-376, 380-387, 389-395, 411-420, 442-457, 463-473, 476-487, 510-521, 531-554, 561-567, 570-585, 604-610, 612-624, 632-651, 660-672, 690-702, 718-730, 738-745, 782-809, 813-820, 823-829, 844-854, 901-906, 911-917, 959-967, 985-991, 997-1006, 1010-1019, 1036-1045, 1053-1059, 1079-1086, 1124-1132, 1137-1150, 1168-1180, 1182-1193, 1209-1214, 1216-1226, 1231-1245, 1271-1277, 1290-1298, 1301-1307, 1339-1345, 22-86, 138-260 and 490-641 of Seq ID No 1007; 37-43, 50-57, 65-82, 86-109, 123-129, 141-150, 152-157, 166-172, 179-203, 209-241, 249-296, 298-307, 312-326, 329-335, 341-348, 364-377, 379-399, 401-409, 411-417, 420-425, 438-444, 461-466, 473-480, 497-505, 522-534, 541-550, 586-597, 608-614, 622-632, 660-666, 679-694, 697-706, 708-731, 737-772, 784-789, 810-825, 837-873, 882-895, 901-928 and 214-233 of Seq ID No 1008; 10-16, 18-27, 41-61, 81-90, 133-142 and 43-118 of Seq ID No 1009; 21-47, 50-69, 83-89, 113-120, 129-146, 152-158, 160-189, 204-225, 235-249, 251-263, 265-275 and 107-140 of Seq ID No 1010; 7-12, 16-24, 28-46, 61-68, 79-85, 87-93, 95-102, 108-123, 148-164, 166-172, 177-202, 205-215, 231-246, 254-261, 267-273, 280-294, 309-315, 322-329, 337-342, 345-351, 386-394, 406-413, 449-455, 473-480, 490-497, 501-508, 532-539, 576-583, 623-629, 657-665, 681-708, 715-722, 751-757 and 584-613 of Seq ID No 1011; 10-26, 52-88, 94-109, 115-172, 175-189, 196-210, 215-232, 244-252, 260-270, 272-283, 293-303, 311-318, 327-339, 348-374, 380-386, 392-402, 421-426, 432-450, 452-460, 466-472, 498-508, 530-537, 569-577 and 258-365 of Seq ID No 1012; 2546, 59-65, 68-75, 83-90, 93-100, 107-115, 124-135, 151-177, 183-189, 194-206, 209-215, 219-224, 251-263, 267-276, 305-311, 318-327, 332-338, 350-356, 380-396, 406-412, 414-423, 431-437, 453-461, 463-481, 483-491, 505-510, 513-523, 528-545, 568-575 and 245-271 of Seq ID No 1013; 4-19, 21-29, 61-68, 87-95, 103-113, 145-154, 157-170 and 10-76 of Seq ID No 1014; 4-18, 28-36, 41-65, 69-84, 96-103, 106-115, 118-124, 126-132, 148-156, 169-181, 187-194, 221-227 and 1-63 of Seq ID No 1015; 5-33, 67-78, 122-129, 141-150, 172-185, 201-209, 217-223, 235-252, 303-316, 355-368, 383-389, 400-406, 411-420, 426-437, 445-451, 459-467, 479-496, 512-517, 523-530, 535-562, 577-584, 590-605, 610-632, 644-654, 663-669, 680-686 and 309-334 of Seq ID No 1016; 31-38, 73-84, 97-121, 124-135, 141-146, 156-170, 174-191, 205-210 and 85-153 of Seq ID No 1017; 24-30, 49-69, 71-111, 113-127, 133-145, 153-169, 172-203, 220-236, 247-254, 256-267, 277-287, 295-302, 311-323, 332-345, 348-358, 364-370, 376-386, 405-410, 416-434, 436-444, 450-456, 482-492, 514-521, 548-561 and 243-286 of Seq ID No 1018; 4-10, 22-31, 39-53, 85-94, 102-110, 130-137, 143-149, 154-161, 174-179, 184-190, 197-203, 206-212, 220-227, 240-248, 251-265, 267-286, 297-310, 338-357, 365-370, 373-382 and 260-345 of Seq ID No 1019; 9-48, 54-71, 84-92, 114-145, 160-188, 190-200, 216-236, 243-255, 261-268, 281-304, 315-322, 330-336, 342-351, 370-380, 383-393, 400-408, 412-420, 436-442, 452-459, 473-480, 501-512, 518-525, 532-541 and 1-94 of Seq ID No 1020; 50-59, 66-81, 90-126, 129-185, 187-205, 213-219, 228-240, 254-266, 268-279, 289-299, 307-314, 323-335, 344-370, 376-382, 388-398, 417-422, 428-446, 448-456, 462-468, 494-504, 526-533, 565-573 and 275-374 of Seq ID No 1021; 5-14, 61-74, 91-99, 110-116, 119-136, 138-149, 159-169, 188-194, 205-227, 236-244, 249-256, 294-305, 321-342, 350-364, 373-378, 385-392, 404-420, 422-430, 462-470, 491-500, 513-520, 583-591, 617-638, 652-663, 674-680, 684-698, 709-718, 745-753, 757-763, 781-788, 835-841, 844-854, 861-869, 882-890, 898-916, 934-940, 946-952, 979-990 and 443-534 of Seq ID No 1022; 23-35, 71-77, 94-100, 134-140, 157-163, 188-212, 214-221, 262-272, 287-293, 323-331, 360-372, 374-380, 402-411, 421-429, 438-443, 462-473, 477-486, 523-528, 530-547, 57-155 and 324-404 of Seq ID No 1023; 11-36, 43-59, 62-70, 78-90, 159-165, 176-188, 197-202, 206-213, 220-225, 234-251, 258-275, 286-293, 300-311, 329-337, 352-361, 363-369, 383-396, 399-417, 420-432, 451-466, 472-478, 504-526, 546-552, 558-566, 576-583, 591-609, 621-628, 642-650, 657-663, 676-681, 692-706, 713-724, 734-753, 763-778, 787-807, 819-825, 57-82 and 445-461 of Seq ID No 1024; 14-26, 38-46, 50-57, 76-87, 89-104, 107-112, 123-134, 136-142, 148-162, 173-194, 200-206, 208-216, 226-233, 243-256, 264-307, 338-343, 351-359, 366-376 and 133-157 of Seq ID No 1025; 13-19, 30-37, 46-60, 75-82, 100-105, 109-115, 134-140, 146-161, 186-192, 199-205, 207-215, 223-230, 254-260, 281-289, 297-311, 344-352 and 251-352 of Seq ID No 1026; 5-16, 19-24, 43-54, 56-89, 104-122, 126-139, 144-157, 165-202, 232-244, 252-265, 272-277, 281-287, 289-300, 308-320, 344-355, 364-390, 396-402, 408-418, 437-442, 448-466, 468-476, 482-488, 514-524, 546-553, 585-593, 284-400 and 486-600 of Seq ID No 1027; 21-27, 29-46, 50-58, 60-70, 76-98, 100-110, 116-122, 124-130, 145-166, 170-188, 199-209, 214-221, 229-236, 244-259, 270-305, 308-314, 319-329, 348-355, 376-383, 396-442, 446-456, 461-466, 479-485, 513-524, 528-533, 539-546, 556-563, 574-585, 595-602, 604-616, 618-625, 630-647, 649-656, 662-674, 680-686, 689-700, 716-739 and 586-630 of Seq ID No 1028; 4-19, 31-49, 82-88, 92-98, 111-141, 146-153, 161-172, 174-197, 199-214, 218-226, 233-239, 242-250, 256-266, 279-293, 298-309, 321-331, 338-345 and 223-248 of Seq ID No 1029; 24-41, 54-61, 63-77, 83-91, 100-111, 116-121, 128-133, 139-146, 153-164, 166-176, 212-242, 255-266, 269-275, 277-285, 290-299 and 144-277 of Seq ID No 1030; 4-29, 33-47, 93-109, 117-125, 152-168, 175-180, 213-219, 224-234, 261-267, 270-284, 321-326, 328-342, 359-365, 383-389, 401-407, 417-424, 471-478, 491-497, 532-537, 545-555, 569-583, 646-652, 688-694, 711-718, 735-745, 794-803, 813-823, 834-839, 851-857, 860-867, 874-882, 895-900, 902-910, 917-923, 933-940, 948-955, 960-966, 1001-1007, 1045-1053, 1058-1066, 1087-1101, 1103-1114, 1130-1139, 1141-1149, 1155-1166, 1192-1198, 1211-1219 and 549-657 of Seq ID No 1031; 13-48, 57-63, 73-81, 89-103, 107-114, 119-125, 130-140, 146-167, 175-186, 202-210, 212-220, 233-239, 255-270, 272-286, 288-301 and 10-23 of Seq ID No 1032; 5-41, 48-56, 75-81, 86-93, 100-111, 126-134, 150-156, 168-188, 209-236, 250-257, 260-274, 291-305, 311-335, 337-344, 373-379, 393-406, 423-450, 461-467 and 105-135 of Seq ID No 1033; 8-16, 37-88, 105-117, 141-169, 172-178, 189-197, 210-217, 243-249, 253-260, 269-282, 293-299, 315-332, 363-373, 378401, 406-415, 417-423, 454-460, 465-471, 503-510, 515-521, 538-576, 578-616, 619-625, 636-643, 651-669, 671-686, 691-698, 719-726, 734-748, 762-778, 782-795, 799-810, 821-845, 894-908, 917-925, 937-943, 946-967, 969-975 and 444-479 of Seq ID No 1034; 31-45, 47-55, 68-82, 87-100, 103-108, 112-117, 134-140, 157-163, 166-175, 178-185, 197-206, 213-219, 234-244, 265-272, 280-289, 292-305, 312-317, 322-328, 335-344, 349-356, 361-372, 376-383, 399-421, 426-444, 455-475, 490-495 and 1-84 of Seq ID No 1035; 4-10, 43-53, 64-100, 116-123, 135-141, 145-154, 164-194, 202-211, 233-242, 250-259, 280-291, 293-314, 318-323, 330-338, 366-379, 381-387, 397-404 and 70-188 of Seq ID No 1036; 4-13, 20-28, 33-49, 61-70, 76-94, 100-146, 151-163, 175-182, 202-212, 218-225, 241-252 and 12-53 of Seq ID No 1037; 12-46, 49-64, 81-97, 103-108, 119-140, 150-173, 179-193, 196-213, 215-223, 225-235, 238-253, 259-264, 267-274, 278-284, 292-305, 314-321, 340-346 and 26-49 of Seq ID No 1038; 18-23, 28-50, 100-150, 157-186, 197-203, 219-231, 233-240, 257-280 and 10-45 of Seq ID No 1039; 10-16, 29-49, 59-73, 79-118, 154-169, 178-192, 204-209, 216-224, 231-242, 250-256, 269-275, 290-311, 319-325, 329-339, 354-365, 371-378, 436-444, 476-485, 507-516, 519-525, 540-547, 556-573, 583-591, 598-604, 606-620, 623-629, 638-648, 653-660, 663-684, 699-704, 707-712, 718-747, 759-765, 777-787, 790-797, 802-808, 826-836, 841-853, 868-876, 879-887, 891-898, 909-916, 922-934, 936-947, 972-994, 1022-1029, 1054-1070, 1084-1090, 1104-1115, 1121-1127, 1146-1155, 1157-1174, 1194-1200, 1207-1215, 1233-1253, 1261-1273, 1286-1307, 1333-1341 and 1054-1156 of Seq ID No 1040; 14-26, 28-37, 40-49, 58-78, 89-95, 107-118, 125-132, 135-142, 155-161, 175-185, 195-213, 215-225, 233-241, 248-254, 263-271, 294-323, 334-340, 345-351, 355-368, 371-394, 401-410, 419-424, 449-456, 463-469, 483-496 and 113-187 of Seq ID No 1041; 10-35, 52-116, 133-143, 149-170, 176-184 and 94-134 of Seq ID No 1042; 4-10, 13-19, 31-39, 57-72, 92-103, 109-122, 126-144, 168-174, 179-192, 234-245, 257-262, 36-62, 157-180 and 190-270 of Seq ID No 1043; 4-29, 36-52, 60-68, 70-90 and 5-73 of Seq ID No 1044; 5-12, 22-56, 58-63, 69-82, 140-146, 175-180, 204-212, 240-248, 276-283, 307-312, 324-329, 335-353, 372-385, 403-412, 436-443, 448-464, 468-474, 476-483, 503-514, 566-573, 590-597, 601-611, 619-626, 630-639, 647-655, 666-679, 689-697, 707-719, 721-728, 761-769, 783-789, 797-803, 806-812, 845-853, 864-870, 893-904, 917-923, 949-956, 967-985, 1005-1021, 1027 1034, 1059-1065 and 1-335 of Seq ID No 1045; 4-16, 18-28, 48-55, 67-75, 81-87, 94-100, 108-124, 139-147, 150-156 and 110-136 of Seq ID No 1046; 4-22, 28-37, 39-46 and 28-38 of Seq ID No 1047; 4-10, 13-19, 27-34, 40-103, 106-113 and 79-100 of Seq ID No 1048; 11-23 of Seq ID No 1049; 4-10, 19-25, 45-51, 64-75, 79-89 and 73-96 of Seq ID No 1050; 4-22, 29-36, 38-47 and 21-44 of Seq ID No 1051; 8-20, 39-44 and 26-42 of Seq ID No 1052; 8-19, 31-47, 49-58, 64-79, 84-96 and 16-50 of Seq ID No 1053; 20-26, 31-37 and 4-13 of Seq ID No 1054; 4-10, 16-32, 34-42, 60-66 and 7-25 of Seq ID No 1055; 4-9, 23-45 and 8-44 of Seq ID No 1056; 4-19, 34-40, 53-81, 103-117, 122-187 and 22-51 of Seq ID No 1057; 4-24 and 1-30 of Seq ID No 1058; 2-27 of Seq ID No 1059; 4-18 and 9-44 of Seq ID No 1060; 4-17, 19-26, 41-49, 63-87, 92-99, 113-131 and 6-105 of Seq ID No 1061; 6-12 and 9-32 of Seq ID No 1062; 4-44, 49-62 and 22-65 of Seq ID No 1063; 20-28, 34-40 and 1-14 of Seq ID No 1064; 4-29, 35-57 and 17-43 of Seq ID No 1065; 14-29 and 24-35 of Seq ID No 1066; 4-19, 31-47, 62-73, 76-83, 87-93, 99-106 and 10-38 of Seq ID No 1067; 4-10, 12-26 and 1-28 of Seq ID No 1068; 16-30, 38-45, 55-61, 69-75, 131-141, 159-165, 169-179, 182-191, 206-214, 230-245, 252-262, 272-280, 299-305, 328-339, 362-370, 372-378, 388-393, 401-409 and 148-226 of Seq ID No 1203; 13-18, 24-33, 36-49, 69-79, 93-101, 113-119, 129-136, 139-164, 167-175, 182-193, 195-217, 224-231, 282-293, 336-345, 351-358, 373-378, 382-387, 407-415, 441-447, 451-459, 461-470, 479-485, 499-526, 533-544, 559-569, 572-581, 583-592, 598-609, 613-624, 632-649, 654-660, 666-694, 700-706, 712-722, 738-752, 771-778, 800-816, 824-839, 847-864, 879-885, 918-925, 927-946, 957-967, 971-999, 1006-1014, 1018-1037, 1046-1055, 1057-1071, 1090-1098, 1104-1113, 1115-1128, 1181-1187, 1193-1202, 1205-1211, 1223-1234, 1240-1248, 1259-1265, 1273-1281, 1289-1301, 1336-1343, 1346-1352, 1356-1363, 1365-1378, 1382-1391, 1401-1408, 1416-1423, 1433-1443, 1450-1455, 1461-1467, 1475-1481, 1486-1493, 1-14 and 1115-1214 of Seq ID No 1204; 6-11, 31-53, 69-84, 86-93, 102-108, 112-121, 135-145, 157-177, 185-202, 208-226, 232-241, 244-251, 260-289, 306-316 and 199-267 of Seq ID No 1205; 4-25, 36-41, 44-49, 68-78, 83-89, 95-102, 113-124, 137-142, 163-175, 179-185, 209-218, 233-244, 250-261, 279-288, 308-322, 330-336 and 49-119 of Seq ID No 1206; 4-24, 42-50, 57-75, 101-107, 109-131, 153-161, 180-190, 214-223, 226-240, 248-265, 197-220 and 252-267 of Seq ID No 1207; 4-24, 56-62, 111-117, 125-130, 138-143, 153-167, 169-175, 195-204, 207-217, 234-250, 284-298, 303-308, 314-326, 351-356, 359-371 and 15-111 of Seq ID No 1208; 431,58-64, 74-80, 88-94, 116-127, 131-138, 141-149, 1-63 and 105-163 of Seq ID No 1209; 4-9, 15-26, 33-40, 57-68, 91-108, 112-124, 132-152, 158-168, 186-213, 228-236, 253-269, 276-283 and 203-222 of Seq ID No 1210; 4-18, 31-48, 50-61, 81-88, 103-110, 114-121, 143-152, 154-164, 176-194, 199-210, 217-225, 234-240, 245-254, 264-279 and 138-153 of Seq ID No 1211; 4-20, 35-41, 51-61, 76-88, 107-115, 122-128 and 13-108 of Seq ID No 1212; 4-18, 25-31, 41-46, 61-70, 79-85, 126-133, 135-141, 166-172, 175-181, 191-199, 208-217, 250-258, 261-273, 312-320, 370-376, 385-394, 401-409, 411-416, 431-445, 457-478, 531-543, 560-567, 575-593, 607-615, 625-633, 679-689, 733-740, 746-752, 157-219 and 432-508 of Seq ID No 1213; 4-28, 47-53, 58-64, 88-97, 124-129, 136-141, 146-167, 174-184, 187-193, 195-203, 205-220, 231-237 and 64-92 of Seq ID No 1214; 7-39, 44-56, 60-65, 83-99, 117-132, 142-148, 162-179, 185-203, 213-231, 239-245, 248-259, 288-295, 330-335, 370-381, 391-396, 401-407, 443-462, 472-492, 505-512, 521-530, 549-557, 562-577, 647-653, 660-666, 673-680, 695-703, 710-717, 729-734, 745-751, 756-766, 769-778, 787-794, 812-819, 825-834, 55-76 and 667-736 of Seq ID No 1215; 5-11, 21-27, 48-56, 64-73, 75-85, 110-117, 124-130, 132-138, 145-163, 165-171, 186-198, 231-239, 246-254, 265-270, 289-296, 312-322 and 100-124 of Seq ID No 1216; 16-24, 26-63, 66-77, 91-97, 100-111, 148-169, 182-188, 205-212, 223-230, 235-244, 264-272, 291-297, 305-312 and 252-296 of Seq ID No 1217; 4-17, 19-31, 34-44, 47-59, 87-93, 99-105, 113-119, 124-137, 139-146, 150-163, 165-175, 185-191, 197-214, 222-227, 235-246, 257-270, 274-279 and 85-152 of Seq ID No 1218; 4-20, 40-52, 74-81, 110-117, 123-138, 144-150, 163-181, 185-195, 199-232, 234-248, 269-277, 280-286, 301-314, 324-329 and 33-55 of Seq ID No 1219; 4-19, 33-55, 67-74, 78-84, 91-105, 109-117, 132-138, 167-176, 190-196, 202-207, 210-217 and 151-162 of Seq ID No 1220; 4-14, 20-26, 28-37, 77-84, 89-94, 114-123, 174-180, 200-208, 222-239, 241-251, 263-271, 276-282, 291-302, 330-335, 374-390, 392-400, 432-441, 447-454, 462-467, 487-501, 516-526, 530-537, 550-566 and 77-110 of Seq ID No 1221; 16-23, 25-31, 42-48, 57-75, 81-95, 108-118, 124-151, 153-170, 178-185, 190-201, 216-222, 230-260, 290-297, 300-307, 312-318, 326-334, 348-363, 365-385 and 89-142 of Seq ID No 1222; 41-50, 52-70, 80-91, 123-131, 136-143, 146-158, 167-178, 207-213, 228-235, 242-264, 274-288, 302-307, 335-347, 349-355 and 59-203 of Seq ID No 1223; 5-14, 21-31, 42-71, 76-103, 111-119, 123-132, 138-144, 167-188, 194-200, 205-212, 230-246, 253-260, 263-269, 275-294, 300-319, 330-343, 349-354 and 335-347 of Seq ID No 1224; 6-14, 18-57, 67-81, 87-97, 104-109, 115-122, 125-139, 147-156, 178-188, 193-199, 211-216, 221-235, 265-287, 293-300, 312-323, 330-352, 369-379, 385-390, 392-404, 408-420, 437-457, 471-483, 489-494, 501-509, 525-535, 547-554 and 410-444 of Seq ID No 1225; 4-18, 55-82, 87-100, 105-117, 145-150, 168-175, 183-189, 198-203, 228-235, 247-256, 277-283, 293-301, 308-321, 337-362, 373-379, 392-397, 427-434, 457-463, 502-510, 539-552, 560-566, 615-629, 99-226 and 566-643 of Seq ID No 1226; 4-27, 59-75, 83-88, 95-105, 112-120, 128-144, 164-169, 200-205, 220-226, 237-242, 253-259, 264-270, 1-103 and 124-232 of Seq ID No 1227; 12-42, 69-77, 103-109, 120-128, 149-157, 161-166, 179-197, 201-218, 238-248, 253-261, 266-272, 278-286, 307-314, 138-216 and 272-287 of Seq ID No 1228; 4-26, 28-43, 50-56, 62-67, 103-109, 121-130, 155-163, 173-179, 197-202, 208-215, 221-233, 258-267, 296-305, 308-314, 321-328, 41-91 and 179-200 of Seq ID No 1229; 4-15, 23-32, 48-55, 61-67, 86-98, 102-111, 124-130, 137-157, 166-171, 178-186, 193-200, 218-232, 236-241, 246-264, 274-288, 291-310, 338-346, 348-359, 389-395, 402-414, 416-428, 430-443, 445-451, 455-469, 473-484, 489-499, 501-509, 511-525, 534-542, 556-561, 579-593, 601-621, 625-637 and 475-513 of Seq ID No 1230; 23-30, 33-50, 53-59, 106-114, 126-132, 140-146, 171-180, 196-204, 224-240, 242-254, 262-272, 274-282, 288-296, 326-332, 341-352, 378-387, 394-407, 412-418, 431-464, 489-496 and 88-176 of Seq ID No 1231; 4-35, 43-52, 60-79, 93-100, 120-139, 146-154, 157-171, 208-226, 234-247, 265-271, 273-283, 292-298, 309-323, 330-339, 355-382, 396-409, 445-453, 455-465, 484-505, 512-525, 528-535, 547-576, 584-594 and 488-559 of Seq ID No 1232; 5-28, 34-41, 47-54, 79-100, 112-117, 122-127, 130-139 and 43-96 of Seq ID No 1233; 4-23, 25-38, 45-50, 68-77, 105-113, 149-156, 177-188, 197-208, 222-229 and 84-145 of Seq ID No 1234; 11-16, 19-32, 37-43, 62-69, 72-80, 82-93, 97-128, 133-142, 156-166, 179-193, 199-205, 209-216, 239-256, 269-278, 295-325, 332-349, 369-379, 6-26 and 347-362 of Seq ID No 1235; 4-10, 17-28, 39-48, 67-82, 93-100, 122-128, 144-153, 177-184, 196-208, 263-285, 289-295, 297-310, 353-359, 367-377, 389-409, 413-421, 426-433, 436-442, 445-451, 453-459, 468-475, 477-488, 506-526, 544-554, 562-611, 639-650, 678-692, 697-706, 718-724, 733-744, 746-754, 766-782, 789-796, 808-822, 831-43, 847-856, 861-868, 887-902, 931-937, 939-951, 957-964, 970-976, 997-1003, 1017-1024, 1033-1039, 1048-1054, 1060-1066, 1075-1083, 1092-1130, 1136-1152, 1168-1176, 1210-1221, 1228-1238, 1242-1247 and 934-1001 of Seq ID No 1236; 73-86, 88-95, 113-118, 166-178, 223-230, 243-280 and 202-216 of Seq ID No 1237; 16-22, 40-46, 51-62, 77-83, 93-106, 116-124, 127-138, 151-158, 162-177, 185-207, 211-220, 223-234, 261-269, 271-280, 309-317, 339-350 and 50-127 of Seq ID No 1238; 11-26, 36-42, 74-85, 88-101, 106-121, 137-156, 186-198, 203-224, 226-254, 295-308, 311-318, 327-355, 373-385, 417-443, 445-464 and 95-163 of Seq ID No 1239; 5-11, 16-23, 34-43, 48-56, 71-83, 89-96, 101-110, 138-162, 164-180, 189-195, 204-214, 222-227, 229-250, 255-261, 272-278 and 19-90 of Seq ID No 1240; 13-19, 26-53, 67-73, 76-82, 87-102, 112-121, 123-148, 151-156, 162-170, 175-186, 199-209, 211-217, 230-244, 251-287, 300-305, 368-374, 403-415, 418-424 and 339-446 of Seq ID No 1241; 4-19, 22-30, 59-73, 84-92, 97-117, 128-140, 165-173, 224-234, 245-255, 276-284, 302-309, 323-330, 349-355, 364-376, 380-385, 391-402, 404-415, 424-432, 437-446, 449-477, 484-499, 516-526, 537-544, 550-555, 562-568, 573-601, 616-623, 653-663, 681-718, 720-728, 762-771, 779-799, 801-806, 814-820, 827-835, 851-870, 899-906, 950-957, 973-990, 995-1005, 1036-1052, 1071-1077, 1086-1098, 1147-1152, 1167-1194 and 866-789 of Seq ID No 1242; 27-55, 57-64, 66-75, 135-142, 172-178, 191-197, 229-235, 241-246 and 184-217 of Seq ID No 1243; 4-24, 33-39, 55-83, 91-114, 126-132, 137-159, 164-189, 202-224, 232-238, 241-253, 259-282, 285-308, 310-335, 359-364, 371-388 and 113-128 of Seq ID No 1244; 6-28, 54-59, 103-114, 122-130, 145-153, 167-175, 184-190, 193-204, 207-212, 215-223, 282-289, 291-300, 315-321, 327-339, 347-354 and 10-103 of Seq ID No 1245; 14-37, 52-57, 73-85, 96-113, 124-136, 142-148, 150-157, 166-172, 182-194, 199-215, 217-224 and 64-160 of Seq ID No 1246; 4-18, 20-33, 59-77, 93-100, 120-129, 131-137, 140-146, 148-158, 166-172, 185-191, 243-249, 253-258, 295-307, 327-333, 345-370, 387-394, 425-432, 483-489, 491-501, 521-527, 538-553, 560-570, 576-582, 629-637, 649-658 and 406-431 of Seq ID No 1247; 9-17, 34-39, 41-59, 71-84, 86-98, 148-169, 177-187, 194-203, 207-214, 222-228, 235-258, 265-273, 286-296, 300-307, 316-328, 338-361, 367-375, 394-401, 407-418, 425-434, 480-495, 502-522, 544-560, 568-575, 584-592, 600-622, 636-641, 661-667, 669-690, 700-707, 719-731, 733-739, 745-750, 767-776, 784-791, 795-811, 840-846, 853-866, 664-745 and 793-856 of Seq ID No 1248; 19-54, 86-91, 98-105, 109-124, 126-136, 145-150, 173-178, 196-204, 212-224, 229-239, 246-252, 279-299, 307-313, 323-329, 337-345, 349-361, 382-393, 399-406, 416-421 and 404-429 of Seq ID No 1249; 20-26, 66-71, 84-97, 105-111, 122-137, 145-165, 170-185, 204-210, 230-242 and 64-158 of Seq ID No 1250; 4-16, 28-69, 72-85, 87-99, 101-123, 128-136, 140-159, 161-173, 185-205, 207-220, 227-240, 242-252, 274-280, 290-296, 301-326, 356-379, 399-453, 461-473, 485-498, 501-525, 527-574 and 117-132 of Seq ID No 1251; 4-11, 37-52, 56-64, 71-82, 89-96, 108-116, 122-137, 144-162, 165-182, 184-194, 228-237, 252-267, 289-296, 473-487, 489-503, 511-516, 527-545, 553-570, 584-593, 604-611, 629-638, 640-649, 684-696 and 536-549 of Seq ID No 1252; 21-37, 81-97, 119-127, 130-143, 158-163, 175-180, 219-230, 245-256, 265-273, 293-299, 319-327, 425-434, 472-485, 493-498, 508-513, 552-557, 562-570, 574-580, 588-594, 597-604, 631-636, 640-646, 667-680, 699-718, 725-747 and 168-235 of Seq ID No 1253; 4-11, 19-29, 51-56, 65-73, 85-98, 109-121, 125-135, 145-161, 171-177, 204-219, 223-228, 252-258, 262-268, 286-313, 315-325, 327-332, 345-352, 395-410, 429-435, 443-451, 455-463, 465-475, 481-487, 516-522, 549-562, 585-591, 598-605, 607-614, 643-651, 673-682, 690-696, 700-705, 725-734, 738-744, 758-765, 769-779, 781-792, 830-844, 847-853 and 700-722 of Seq ID No 1254; 1040, 68-91, 95-104, 140-152, 158-170, 185-191, 196-201, 205-219, 240-246, 249-256, 262-268, 274-280, 293-313, 315-320, 326-332, 338-358, 402-453, 457-466, 476-516 and 31-94 of Seq ID No 1255; 10-34, 41-47, 50-66, 73-92, 100-107, 121-127, 133-139, 146-155, 159-175, 184-191, 223-230, 238-247, 273-286, 300-310, 328-336, 352-358, 365-372, 376-409, 415-423, 446-452, 459-465, 471-484, 509-522, 532-540, 543-554, 586-598, 600-610, 617-632, 670-689, 695-706, 711-727, 741-746, 752-760, 772-835, 843-882, 890-933, 958-973, 991-1022, 1024-1048, 1053-1070 and 277-365 of Seq ID No 1256; 4-31, 56-70, 77-96, 112-117, 124-137, 139-155, 160-169, 176-193, 228-234, 237-257, 271-288, 317-322, 337-371, 373-391 and 317-353 of Seq ID No 1257; 4-14, 20-28, 30-52, 54-62, 76-84, 94-100, 125-132, 140-181, 185-191, 208-222 and 181-199 of Seq ID No 1258; 9-46, 53-59, 74-80, 82-93, 97-103, 111-117, 130-142, 152-161, 188-197, 236-251, 264-271, 285-295, 307-340, 343-394, 397-412, 416-436, 440-472, 497-527, 540-548, 561-566, 573-583, 591-598, 607-616, 624-631, 639-649, 669-675, 683-689, 694-718, 728-736, 756-771, 782-803, 814-829, 831-871, 873-879, 882-897, 900-912, 920-928, 940-953, 963-998 and 243-318 of Seq ID No 1259; 5-16, 24-30, 34-39, 45-51, 59-72, 80-86, 100-108, 116-123, 133-140, 148-162, 176-181, 184-211, 219-228, 233-242, 257-278, 300-310, 320-338, 340-345, 352-360 and 226-320 of Seq ID No 1260; 5-12, 51-58, 61-74, 95-112, 124-137, 153-158, 163-189, 192-204, 209-236, 240-250, 255-273, 304-317, 320-326, 334-348, 350-356, 360-378, 384-416, 439-457, 465-470, 488-493, 496-505, 531-541, 548-557, 579-587, 593-601, 616-647, 649-659, 679-685, 693-702, 705-713, 715-734, 737-743, 751-758, 763-779, 781-788, 791-801, 856-862, 882-896, 903-914 and 38-55 of Seq ID No 1261; 8-34, 51-57, 68-76, 79-95, 110-116, 127-140, 142-154, 162-172, 174-202, 214-220, 228-239, 292-306, 314-320, 322-329, 337-344, 356-371, 374-380, 382-393, 416-421, 424-433, 444-453, 461-468, 470-478, 485-490, 497-503, 511-519, 537-543, 555-564, 566-579, 588-594, 603-609, 615-627, 63-640, 647-659, 663-689, 699-710, 728-735, 739-748, 750-756, 764-769, 776-788, 418-441 and 511-591 of Seq ID No 1262; 12-19, 21-30, 32-39, 43-49, 55-68, 76-87, 97-104, 114-123, 130-141, 153-168, 179-205, 207-216, 218-226 and 223-239 of Seq ID No 1263; 8-34, 72-121, 128-141, 153-172, 174-216, 221-256, 261-294, 307-315, 332-349, 354-370, 372-435, 452-457, 461-477, 487-492, 503-509, 511-520, 537-549, 559-565, 568-582, 584-591, 593-602, 607-617, 625-638, 655-674, 681-687, 696-703 and 254-268 of Seq ID No 1264; 4-13, 23-40, 79-98, 106-124, 126-149, 154-161, 163-176, 178-187, 199-226, 232-254, 256-276, 297-304, 308-326, 329-338, 364-373, 384-399, 404-432, 439-499, 502-518, 523-544, 557-568, 571-582, 584-590, 603-617, 621-627, 633-639, 641-651, 653-663, 675-684, 686-699, 705-729, 736-781 and 522-597 of Seq ID No 1265; 5-45, 49-62, 85-99, 114-122, 136-148, 151-171, 198-211, 253-260, 278-287, 297-303, 309-314, 318-324, 326-336, 348-363 and 1-115 of Seq ID No 1266; 5-13, 22-33, 88-95, 132-144, 160-166, 189-202, 210-223, 253-258, 269-282, 286-294, 83-98 and 244-269 of Seq ID No 1267; 18-25, 29-38, 72-95, 97-107, 110-139, 144-152, 155-161, 174-182, 198-203 and 44-56 of Seq ID No 1268; 5-14, 17-23, 29-50, 52-64, 72-98, 109-115, 120-135, 137-145, 152-158, 167-175, 178-185, 210-234, 241-255, 258-271, 276-281, 290-303, 307-312 and 10-34 of Seq ID No 1269;5-32, 50-56, 62-70, 78-84, 97-121, 132-171, 177-182, 188-195, 204-214, 241-250, 267-274, 276-281, 292-309, 311-320, 333-344, 349-361, 375-395, 398-405 and 383-400 of Seq ID No 1270; 4-10, 16-26, 59-80, 82-93, 97-115, 117-131, 148-160, 169-182, 184-210, 217-234, 241-254, 256-263, 265-276, 306-312, 344-350, 384-395, 400-409, 416-423, 428-440, 449-465, 496-504, 517-555, 335-360 and 427-541 of Seq ID No 1271; 4-20, 48-91, 96-115, 134-142, 171-187, 197-217, 222-242, 246-255, 264-270, 277-289, 305-320, 338-352, 354-373 and 21-130 of Seq ID No 1272; 6-23, 25-53, 61-76, 83-92, 107-121, 147-166, 186-201, 207-215, 243-251, 264-274, 282-326, 333-348, 357-366, 371-380, 401-423, 432-465, 471-477, 481-490, 500-506, 512-525, 540-560, 583-603, 605-612, 615-626, 647-656, 661-681, 687-693, 713-722 and 587-614 of Seq ID No 1273; 4-9, 15-21, 28-35, 39-54, 59-73, 76 86, 92-108, 120-134, 136-142, 145-161, 209-217, 220-228, 236-249, 258-267, 275-282, 293-304, 306-327, 330-340, 346-352, 354-360, 368-383, 386-392, 401-413 and 97-175 of Seq ID No 1274; 4-11, 20-30, 47-66, 72-97, 108-117, 119-129, 142-163, 211-219, 224-237, 243-249, 251-263, 270-288, 295-305, 311-316, 326-333, 341-346, 367-375 and 301-378 of Seq ID No 1275; 22-30, 38-45, 62-68, 78-90, 96-103, 107-114, 118-127, 134-148, 150-173, 179-193, 195-200, 205-219, 221-234, 239-248, 250-280, 282-296, 308-325, 334-351, 363-389, 425-432, 438-443, 468-481, 488-495, 499-517, 570-593, 602-610, 613-621, 629-637 and 536-559 of Seq ID No 1276; 12-23, 26-35, 51-63, 72-78, 86-91, 135-140, 183-190, 201-209, 211-219, 241-247, 260-266, 272-281, 295-301, 329-335, 339-345, 355-366, 387-402, 428-445, 453-459, 503-517, 519-527, 531-538, 546-553, 560-569, 3-131 and 232-331 of Seq ID No 1277; 8-13, 25-35, 51-56, 72-78, 86-91, 135-140, 183-190, 201-209, 211-219, 241-247, 260-265, 272-281, 295-301, 329-335, 339-345, 355-366, 387-402, 428-445, 453-459, 503-512, 519-529, 531-538, 546-553, 560-569, 1-145, 160-183, 212-334 and 376400 of Seq ID No 1278; 5-17, 62-71, 73-116, 118-131, 137-144, 151-158, 160 167, 169-175, 181-190, 193-210, 212-222, 231-262, 273-280, 300-329, 341-358, 363-368, 394-400, 403-409, 416-427, 450-437, 464-470, 478-484, 499-511, 513-529, 544-554, 558-565, 573-589, 597-604 and 335-348 of Seq ID No 1279; 4-18, 38-46, 52-60, 65-79, 93-115, 123-131, 144-154, 168-183, 191-196, 201-223, 225-236, 250-263, 273-278, 289-317, 328-338, 357-373, 384-399 and 159-248 of Seq ID No 1280; 11-39, 43-52, 57-69, 72-98, 112-142, 147-154, 159-165, 167-178, 198-210, 213-227, 244-250, 257-266, 268-286, 295-301, 305-311, 318-338, 340-346 and 262-323 of Seq ID No 1281; 4-9, 17-23, 40-49, 57-70, 72-87, 92-121, 124-133, 135-146, 158-164, 173-195, 204-213, 215-221, 230-246, 250-257, 260-273, 280-298, 304-309, 311-328, 336-343, 362-371, 373-406, 409-418, 433-446, 450-456, 490-496, 503-513, 526-542, 548-563, 569-592 and 185-212 of Seq ID No 1282; 4-17, 23-34, 36-44, 53-62, 72-85, 87-92, 110-115, 118-123, 129-150, 155-168, 172-180, 191-197, 205-211, 213-223, 225-233 and 176-201 of Seq ID No 1283; 8-26, 33-44, 52-72, 78-96, 145-151, 154-171, 204-212, 223-230, 236-251, 261-272, 280-341, 365-374, 385-394, 417-423, 434-447, 456-486, 494-500, 509-519, 530-546, 556-566, 568-579, 581-603 and 300-322 of Seq ID No 1284; 5-11, 14-21, 26-45, 52-67, 71-79, 82-97, 104-144, 151-159, 183-189, 194-205, 211-223, 241-252, 265-273, 275-280 and 93-116 of Seq ID No 1285; 13-20, 55-72, 102-109 and 10-107 of Seq ID No 1286; 5-14, 16-22, 38-45, 51-59, 61-78, 94-102, 133-142, 153-160, 187-195, 208-221, 240-254, 256-262, 270-275, 281-287, 294-299, 338-354, 356-364, 369-378, 446-452, 506-515, 557-564, 576-599 and 132-242 of Seq ID No 1287; 4-29, 41-57, 66-75, 86-93, 96-102, 109-116, 124-131, 164-171, 188-194, 199-208, 214-231, 289-295, 305-310, 314-319, 336-351, 362-368, 389-399, 403-412, 422-433, 435-441, 444-461, 463-469 and 250-326 of Seq ID No 1288; 4-16, 46-56, 59-73, 85-94, 97-105, 127-144, 160-166, 188-194, 233-238, 245-250, 270-275, 286-291 and 59-76 of Seq ID No 1289; 5-33, 58-64, 78-116, 119-127, 139-160, 171-190 and 33-56 of Seq ID No 1290; 6-11, 19-39, 50-56, 60-66, 83-133, 135-167, 195-216, 226-260, 271-319, 327-339, 342-355, 362-368, 373-394 and 170-191 of Seq ID No 1291; 4-37, 45-51, 106-119, 132-138, 150-156, 160-171, 176-182, 193-203, 207-215, 222-229, 237-244, 253-261, 296-372, 403-409, 416-422, 440-449, 451-460, 471-485, 504-510, 538-551, 560-569, 573-582, 585-597, 606-617, 632-646, 658-666, 668-676, 685-694 and 552-658 of Seq ID No 1292; 16-32, 53-79, 81-96, 102-111, 124-130, 145-152, 159-168, 176-189, 206-231, 236-244, 251-258, 286-292, 304-310, 315-326, 351-361, 383-390, 392-398, 405-411, 430-436, 456-464, 470-480, 482-496, 505-513, 531-540, 546-558, 583-589, 601-607, 621-634, 638-644, 667-673, 681-687, 693-702, 721-733, 737-744, 747-757, 760-767, 772-789, 796-807, 818-823, 846-852, 856-866, 868-880, 882-890, 913-919, 923-929 and 14-34 of Seq ID No 1293; 19-42, 55-67, 75-95, 144-156, 168-175, 183-189 and 98-109 of Seq ID No 1294; 7-17, 22-27, 34-61, 88-97, 110-117, 152-159, 175-191, 202-213, 220-232, 267-285, 296-315, 341-347, 376-392, 400-408, 421-430, 453-462, 464-470, 478-485 and 199-269 of Seq ID No 1295; 27-44, 71-80, 114-123, 127-140, 149-167, 175-188, 191-202, 205-217, 222-227, 270-276, 297-302, 308-318, 324-332, 349-368, 370-376, 382-393, 432-441, 445-459, 472-481, 489-496 and 3-25 of Seq ID No 1296; 16-49, 57-63, 84-92, 100-120, 124-130, 160-183, 189-200, 202-209, 236-244, 263-280, 283-334, 341-346, 377-389, 405-423, 452-458, 485-493, 505-513, 518-530, 547-559, 568-579, 595-601, 619-625, 638-649 and 474-504 of Seq ID No 1297; 4-18, 21-30, 43-54, 76-83, 85-106, 116-122, 124-160, 180-185, 198-204, 206-222, 230-241, 258-263, 270-302, 325-332, 359-371, 374-386, 391-402, 411-417, 435-443, 458-485 and 376-395 of Seq ID No 1298; 17-26, 28-34, 54-62, 83-93, 108-114, 119-125, 151-164, 175-187, 197-222, 224-232, 234-250, 265-270, 276-313, 315-342, 373-383 and 132-153 of Seq ID No 1299; 25-42, 47-53, 55-80, 86-113, 116-126, 128-135, 151-159, 167-174, 179-188, 207-221, 226-248, 257-266, 269-279, 297-304, 312-325 and 144-155 of Seq ID No 1300; 16-26, 61-71, 75-85, 95-113, 126-163, 175-184, 202-246, 286-291, 293-302, 320-349 and 57-76 of Seq ID No 1301; 4-19, 29-36, 48-59, 92-103, 117-139, 154-164, 190-201 and 87-113 of Seq ID No 1302; 43-51, 98-108, 123-130, 149-159, 168-179, 182-188, 201-207, 231-240, 334-340, 346-354, 369-378, 462-472, 493-503, 510-518, 520-538, 565-572, 584-590, 592-598, 616-628, 637-646, 659-675, 688-694, 699-705, 724-732, 740-755, 771-780, 792-801, 827-833, 846-856, 873-880, 882-906, 676-694 and 855-881 of Seq ID No 1303; 15-45 and 29-60 of Seq ID No 1304; 4-11, 15-34, 49-77, 92-121, 128-139, 169-176, 181-210, 216-222, 225-231, 233-246, 248-264, 271-276, 282-306, 319-326, 346-352 and 250-277 of Seq ID No 1305; 6-20, 46-54, 64-79, 81-87, 131-138, 156-162, 170-178, 190-198, 226-232, 254-263, 266-274, 293-303, 313-323, 363-369, 419-424, 426-432, 436-455, 507-513, 515-521, 534-547, 553-559, 596-610, 617-636, 638-647, 659-665, 677-683, 691-696, 710-718, 724-730, 750-755, 770-780, 802-808, 824-842, 857-862 and 436-529 of Seq ID No 1306; 11-25 and 21-35 of Seq ID No 1307; 1-12 of Seq ID No 1308; 4-10 and 2-11 of Seq ID No 1309; 12-20 and 5-14 of Seq ID No 1310; 3-10 of Seq ID No 1311; 4-22 and 10-21 of Seq ID No 1312; 4-30, 35-50 and 17-32 of Seq ID No 1313; 1-20 of Seq ID No 1314; 16-21 and 7-22 of Seq ID No 1315; 5-13 and 7-18 of Seq ID No 1316; 10-17 and 16-29 of Seq ID No 1317; 17-27 and 22-34 of Seq ID No 1318; 12-18 and 9-21 of Seq ID No 1319; 4-11, 13-94, 100-111, 115-134 and 89-106 of Seq ID No 1320; 4-24 and 10-27 of Seq ID No 1321; 4-14 and 12-23 of Seq ID No 1322; 18-33 and 13-33 of Seq ID No 1323; 16-23,2548 and 29-40 of Seq ID No 1324; 4-12, 25-64, 68-76 and 4-61 of Seq ID No 1325; 4-57, 59-66, 96-104 and 76-91 of Seq ID No 1326; 23-43, 45-75, 97-107, 112-121 and 26-62 of Seq ID No 1327; 10-24 of Seq ID No 1328; 4-50 and 30-44 of Seq ID No 1329; 4-20, 24-43 and 10-24 of Seq ID No 1330; 4-49, 56-66 and 5-54 of Seq ID No 1331; 33-44 and 15-38 of Seq ID No 1332; 4-36, 39-50 and 32-47 of Seq ID No 1333; 4-9, 16-30, 32-38 and 15-32 of Seq ID No 1334; 4-9 and 27-42 of Seq ID No 1335; 4-16, 32-43, 49-58, 64-72 and 14-27 of Seq ID No 1336; 12-19, 24-29, 37-43, 47-53, 65-72, 83-95, 112-122, 136-147, 162-168, 174-181, 189-195, 201-208, 216-221, 234-243, 270-276, 278-288, 305-316, 318-342, 350-356, 368-400, 420-428, 434-443, 471-477, 481-488, 530-535, 540-547, 566-575, 591-601, 603-609 and 624-629 of Seq ID No 763; 11-22, 28-34, 40-45, 65-86, 99-107, 115-125, 132-141, 143-150, 158-190, 203-211, 216-239, 246-257, 259-270, 272-279, 286-306, 313-332, 338-364, 371-380, 389-397, 410-418, 422-435, 467-510, 515-521, 532-538 and 547-563 of Seq ID No 764; 7-20, 28-45, 51-66, 81-104, 108-115, 124-137, 149-155, 161-206, 209-214, 222-239, 250-262, 274-282, 309-343, 351-363, 365-386, 405-413, 435-440, 446-454, 458-466, 470-477 and 482-492 of Seq ID No 765; 10-25, 29-35, 39-46, 54-71, 82-88, 102-111, 122-137, 139-145, 152-160, 162-172, 176-182, 193-201, 209-218, 226-232, 242-249, 258-268, 299-314, 318-344, 362-376, 393-399, 405-418, 426-463, 473-485, 487-492, 498-503, 518-544 and 561-567 of Seq ID No 766; 16-23, 66-90, 98-110, 125-131, 144-150, 194-200, 213-219, 221-232, 237-256, 263-281, 293-298, 311-318, 326-337, 339-354, 373-389, 396-402, 404-421, 427-439, 441-448, 452-462, 467-479, 508-530, 534-541, 544-550, 562-569, 575-581, 583-592, 595-628, 636-656, 658-672, 674-680, 687-697, 715-721, 731-736, 739-749, 754-761, 771-788, 790-797 and 813-824 of Seq ID No 767; 14-42, 51-57, 66-77, 84-96, 103-111, 129-148, 158-193, 198-208, 212-222 and 242-262 of Seq ID No 768; 4-23, 29-62, 65-84, 98-104, 128-135, 144-161, 167-173, 175-204, 219-240, 250-264, 266-278 and 280-290 of Seq ID No 769; 13-26, 33-45, 50-60, 75-81, 97-105, 123-131, 138-145, 158-166 and 168-177 of Seq ID No 770; 6-20, 23-44, 50-61, 67-82, 84-91, 104-125, 133-144, 149-156, 159-166, 173-180, 182-196, 200-206, 224-239, 245-288, 320-339, 342-349, 359-368, 377-385, 411-419, 427-435, 453-474, 481-489, 491-497, 505-514, 516-522, 536-564, 579-586, 618-631, 644-650, 654-661, 663-677, 679-689, 700-705, 710-753, 795-801, 809-814, 821-827, 842-851, 867-905, 920-925, 931-949, 954-961, 1017-1022, 1034-1040, 1047-1057, 1062-1075, 1081-1086, 1107-1117, 1119-1126, 1145-1154 and 1162-1172 of Seq ID No 771; 13-21, 47-57, 72-83, 97-102, 105-119, 125-133, 146-153, 170-177, 221-239, 245-273, 283-291, 299-305, 317-329, 335-343, 358-367, 374-380, 399-407, 430-438, 449-454, 473-479, 483-505, 517-527, 531-537, 555-560, 586-599, 601-616, 623-629, 639-647, 649-654, 658-667, 669-676, 690-709 and 714-729 of Seq ID No 772; 1428, 34-40, 45-54, 69-76, 78-83, 86-100, 116-123, 135-143, 146-161, 168-179, 187-200, 204-225, 236-250, 255-265, 271-292 and 298-314 of Seq ID No 773; 4-28, 36-42, 78-85, 106-122, 130-136, 144-150, 161-175, 180-190, 194-200, 226-234, 256-265, 274-294, 309-316, 324-333, 336-344, 373-379, 382-389, 398-404, 407-416, 422-446, 451-462 and 530-541 of Seq ID No 774; 4-15, 17-42, 71-77, 80-86, 90-116, 123-135, 144-150, 153-163 and 183-194 of Seq ID No 775; 7-13, 22-42, 56-62, 84-90, 102-112, 121-133, 140-148, 158-167, 173-181, 192-199, 227-234, 284-293, 301-307, 336-343, 345-353, 366-372, 376-397, 400-436, 439-450, 467-478, 504-510, 519-530, 532-547, 551-558, 564-575, 592-598, 619-630, 636-642, 655-661, 663-669, 671-679, 697-718, 724-736, 738-752, 759-773, 776-788, 805-823, 827-833, 842-852, 859-864, 874-881, 883-889, 905-914, 932-939, 941-957, 963-969, 978-991, 1011-1025, 1052-1062, 1067-1073, 1080-1088, 1106-1112, 1121-1132 and 1139-1152 of Seq ID No 776; 9-23, 32-38, 44-54, 64-71, 95-126, 146-154, 163-173, 190-196, 199-206, 249-256, 262-277, 291-302, 350-356, 380-398, 406-413, 420-430, 433-444, 471-478 and 492-507 of Seq ID No 777; 5-26, 57-66, 68-74, 81-87, 105-116, 122-132, 144-160, 185-212, 217-223, 228-234, 241-252, 269-274, 291-313, 318-328, 335-342, 368-375, 397-404, 411-422, 431-439, 462-472, 474-485, 493-499, 509-519, 521-527, 530-558, 563-579 and 590-602 of Seq ID No 778; 9-23, 32-38, 44-54, 64-71, 95-126, 146-154, 163-173, 190-196, 199-206, 249-256, 262-277, 291-302, 350-356, 380-398, 406-413, 420-430, 433-444, 471-478 and 492-507 of Seq ID No 779; 18-31, 34-53, 57-67, 74-81, 90-106, 136-144, 147-153, 157-163, 170-182, 192-207, 233-241, 245-251, 256-267, 274-281, 284-306, 318-330, 333-340, 345-351, 356-379, 388-404, 428-439, 455-466, 468-480, 488-505, 515-526, 553-564, 571-577, 594-600, 607-614, 616-628, 634-642, 644-651, 655-666, 672-678, 686-703, 732-738, 745-751, 755-768, 772-778, 785-805, 807-814, 817-825, 831-853, 858-868, 890-905, 918-926, 934-942, 957-970, 972-981, 990-1001, 1057-1067, 1069-1077, 1089-1109, 1116-1130, 1133-1141, 1154-1165, 1190-1206, 1208-1215, 1217-1225, 1228-1254, 1256-1263, 1271-1279, 1283-1305, 1333-1339, 1357-1367, 1373-1379, 1388-1405, 1432-1442, 1444-1451, 1453-1461, 1463-1479, 1488-1504, 1516-1524, 1532-1540, 1555-1568, 1589-1600, 1607-1613, 1633-1638, 1655-1665, 1687-1704, 1721-1728, 1731-1737, 1744-1763, 1793-1804, 1816-1823, 1833-1850, 1855-1865, 1868-1875, 1886-1902, 1916-1925, 1931-1937, 1954-1967, 1971-1977, 1987-2002, 2009-2015, 2030-2036, 2043-2049, 2053-2077, 2085-2098, 2114-2122, 2129-2136, 2154-2167, 2187-2203, 2232-2243, 2253-2274, 2286-2303, 2311-2323, 2344-2365, 2371-2378, 2388-2404, 2406-2413, 2415-2424, 2426-2450, 2454-2461, 2469-2475 and 2486-2505 of Seq ID No 780; 4-22, 24-30, 33-39, 60-72, 83-89, 128-137, 153-161, 165-174, 186-194, 212-218, 251-260, 275-284, 288-297, 309-318, 335-341, 374-383, 399-407, 411-420, 432-440, 467-482, 518-526, 572-579, 595-609, 649-663, 680-686, 691-702 and 708-714 of Seq ID No 781; 11-25, 39-57, 69-94, 100-107, 118-155, 158-171, 189-201, 226-233, 236-245, 249-262, 287-296, 298-312, 315-329, 333-342, 351-359, 364-374, 382-388, 399-407, 411-417, 419-449, 454-471, 486-492, 494-504, 515-541, 547-552, 582-600, 611-623, 625-641, 651-657, 678-692, 699-709, 713-720, 746-752, 772-781, 791-804, 829-844, 880-893, 900-910, 915-923, 936-942 and 953-970 of Seq ID No 782; 5-11, 17-24, 26-32, 36-43, 50-61, 67-73, 91-102, 111-126, 133-148, 154-161, 167-173, 179-195, 208-223, 230-240, 242-253, 270-286, 292-306, 308-347, 352-371, 373-380, 386-395, 404-410, 418-433, 436-444, 447-460, 463-477, 486-492, 522-533 and 548-553 of Seq ID No 783; 4-12, 15-27, 35-55, 68-95, 100-109, 117-122, 129-135 and 157-162 of Seq ID No 784; 25-46, 59-65, 68-75, 83-90, 93-100, 107-115, 124-135, 151-177, 183-189, 194-206, 209-215, 219-224, 251-263, 267-276, 305-311, 318-327, 332-338, 350-356, 380-396, 406-412, 414-423, 431-437, 453-461, 463-481, 483-491, 505-510, 513-523, 528-545 and 568-575 of Seq ID No 785; 10-35, 42-59, 65-70, 76-85, 92-104, 149-155, 184-191, 234-243, 248-259, 268-277, 279-287, 391-398, 410-430, 445-454, 488-494, 498-504, 518-523, 530-538, 574-590, 615-623, 627-633, 652-660, 662-670, 674-683, 703-714, 720-728, 731-737 and 751-757 of Seq ID No 786; 5-12, 39-51, 57-64, 67-84, 86-108, 124-130, 139-159, 167-179, 181-202 and 226-235 of Seq ID No 787; 12-20, 29-40, 57-77, 79-88, 97-103, 111-117, 119-137, 174-200, 202-218, 221-229, 231-238, 240-246, 254-264, 266-280, 296-308 and 321-331 of Seq ID No 788; 4-18, 20-48, 54-68, 80-105, 110-117, 120-130, 132-167, 179-214, 227-246, 259-295, 306-323, 332-339, 345-351, 357-363, 366-374 and 379-392 of Seq ID No 789; 8-21, 23-31, 53-66, 69-94, 99-113, 119-184, 190-214, 233-244, 268-274, 279-284, 289-298, 300-311, 315-337, 344-350, 364-383 and 385-397 of Seq ID No 790; 26-38, 43-63, 67-76, 79-98, 105-112, 115-121, 132-144, 148-153, 179-184, 194-203, 239-245, 261-278 and 282-315 of Seq ID No 791; 13-22, 24-30, 49-61, 65-72, 90-97, 99-105, 115-131, 152-160, 165-171, 176-188, 202-221, 231-250, 255-274, 280-286, 288-296, 331-337, 339-347, 350-358, 374-385, 391-408, 418-427, 438-453, 468-476, 482-490, 497-506, 526-532, 534-583, 696-702, 713-719, 730-748, 750-758, 762-778, 802-808, 825-857, 864-950, 963-1004, 1015-1023 and 1046-1058 of Seq ID No 792; 4-10, 22-28, 35-44, 58-66, 74-84, 86-98, 116-131, 138-143, 181-186, 224-230, 241-253, 282-292, 305-313, 325-331, 333-341, 348-360, 384-391, 395-408, 415-429, 431-445, 525-531, 558-564, 567-584, 601-612, 624-637, 645-652, 682-687 and 700-706 of Seq ID No 793; 4-13, 19-27, 33-40, 57-82, 107-115, 117-125, 162-173, 197-206, 215-226, 256-266, 291-298, 303-310, 318-323, 331-336, 345-360, 379-396, 405-410, 426-434, 454-462, 468-476, 482-497, 512-518, 529-541, 556-564, 570-581, 589-594, 601-610, 629-637, 639-648, 663-670, 704-711, 725-738, 746-768, 770-780, 787-804, 817-823, 830-837, 876-882, 930-936 and 939-968 of Seq ID No 794; 5-13, 20-65, 67-74, 107-115, 128-169, 171-195, 238-244, 256-287 and 292-298 of Seq ID No 795; 7-13, 16-77, 89-96, 104-114, 117-125, 148-160, 167-191, 193-202 and 227-236 of Seq ID No 796; 21-36, 41-47, 54-89, 122-129, 138-165, 173-190, 196-216 and 221-229 of Seq ID No 797; 8-45, 135-140, 172-182, 189-196, 206-216, 218-235, 260-269, 272-278, 307-313, 333-344, 352-359, 371-395, 403-414, 416-422, 426-438, 451-470, 478-484, 493-502, 504-511 and 514-533 of Seq ID No 798; 6-25, 49-59, 65-96, 107-115, 117-124, 135-151, 176-185 and 203-209 of Seq ID No 799; 5-15, 46-56, 58-81, 83-111, 118-138, 152-160 and 165-175 of Seq ID No 800; 7-16, 18-24, 36-43, 54-60, 65-73, 88-94, 107-113, 122-128, 134-141, 162-171, 182-216, 218-235, 249-263, 266-278, 290-301 and 308-338 of Seq ID No 801; 4-14, 19-24, 27-36, 38-51, 63-73, 90-96, 102-121, 138-150, 157-174, 176-202, 212-225, 229-245, 250-258, 261-268, 279-291, 293-310, 319-338, 358-368, 371-389, 393-398, 404-413, 416-433, 435-442 and 458-471 of Seq ID No 802; 17-40, 47-82, 85-93, 101-113, 153-172, 180-186, 190-208, 215-224, 252-261, 269-279, 283-289, 294-306, 311-328, 397-408, 416-423, 425-437, 492-499, 513-534, 542-548 and 550-555 of Seq ID No 803; 8-17, 29-43, 45-52, 58-69, 87-100, 102-112, 148-163, 172-187, 190-208, 210-227, 232-239, 245-253, 258-263, 286-299, 313-334, 346-362, 373-388, 391-410, 417-423, 425-430, 434-446, 457-472, 483-489, 496-502, 518-524, 537-546, 555-560, 602-610, 637-646, 676-689, 698-704, 706-742, 750-778, 780-791, 806-842, 864-879, 881-888, 890-899, 901-908, 910-921, 941-947, 953-959, 967-980, 990-995, 1000-1061, 1073-1079, 1081-1092, 1096-1118, 1121-1168, 1174-1185, 1195-1209, 1219-1232, 1237-1243, 1250-1274, 1276-1286, 1302-1319, 1324-1333, 1339-1344, 1349-1361, 1370-1376, 1418-1427, 1435-1449, 1453-1469, 1473-1478, 1482-1495, 1509-1517 and 1519-1526 of Seq ID No 804; 4-14, 16-32, 42-47, 65-71, 82-109, 128-145, 158-171, 177-191, 197-228 and 230-236 of Seq ID No 805; 16-22, 26-42, 52-72, 75-89, 97-102, 114-147, 154-160, 165-170, 172-200, 202-229, 231-244, 256-261, 267-278, 286-294, 312-319, 330-336, 340-346, 360-373, 375-383, 386-396, 420-441, 443-474, 484-491, 496-517, 535-574, 600-606, 608-624, 636-643, 646-658, 664-687, 692-703, 716-725, 733-750 and 755-764 of Seq ID No 806; 4-14, 24-34, 47-69, 81-90, 98-112, 144-153, 161-169, 189-196, 202-208, 213-220, 243-249, 256-262, 265-271, 279-286, 299-307, 310-324, 326-345, 356-369, 397-416, 424-429 and 432-441 of Seq ID No 807; 4-9, 14-23, 50-56, 59-68, 77-102, 111-120, 126-152, 161-167, 174-180, 189-202, 204-228, 237-245, 259-266, 278-285 and 300-309 of Seq ID No 808; 23-35, 71-77, 94-100, 134-140, 157-163, 189-195, 211-219, 221-231, 238-244, 246-253, 263-277, 298-306, 315-321, 337-342, 350-355, 369-377, 389-400, 408-416, 422-427, 441-449, 465-477, 481-488, 527-532 and 534-551 of Seq ID No 809; 14-20, 35-48, 53-63, 71-77, 95-101, 114-121, 123-133, 144-151, 153-160, 162-170, 190-197, and 201-211 of Seq ID No 810; 9-17, 26-46, 65-72 and 90-101 of Seq ID No 811; 21-39, 46-53, 68-96, 107-113, 118-124, 126-135, 158-185, 196-202, 204-213, 219-226, 246-253, 267-275, 277-285, 299-317, 319-338, 404-410, 421-428 and 435-463 of Seq ID No 812; 17-24, 29-40 and 47-56 of Seq ID No 813; 17-25, 32-77, 82-91, 100-128, 161-169, 194-207, 211-218, 227-232, 239-245, 255-260, 278-300, 311-325, 342-356, 382-390, 393-401, 416-460, 467-487, 491-503, 505-512, 516-532, 551-565, 568-575, 594-601, 610-632, 638-643, 647-670, 672-685, 699-710 and 712-726 of Seq ID No 814; 5-14, 25-46, 49-55, 59-65, 77-85, 98-107, 125-169, 181-186, 223-240, 271-281, 290-300, 306-313, 315-322, 330-337, 348-359, 370-377, 384-392, 416-424, 428-445, 456-469, 479-486, 502-510, 518-523, 525-535, 555-575, 578-585, 605-612, 624-637, 661-685, 693-700, 708-721, 723-729, 744-754 and 770-775 of Seq ID No 815; 4-33, 38-52, 92-106, 116-124, 133-138, 142-148, 153-159, 161-168, 245-257, 282-287, 314-321, 331-336, 355-361, 366-372, 374-390, 396-409, 447-455, 484-490, 498-504, 511-519, 531-538, 540-545, 574-581, 586-596, 625-631, 644-655, 668-674, 685-692, 718-723, 728-741, 771-778, 787-797, 801-807, 819-828 and 832-844 of Seq ID No 816; 5-25, 31-39, 72-79, 93-102, 104-110, 122-132, 138-146, 157-189, 192-198, 205-214, 226-233, 240-248, 269-275, 282-298, 304-310, 313-327 and 342-348 of Seq ID No 817; 12-39, 49-55, 59-69, 95-104, 106-111, 116-128, 161-184, 186-217, 229-237, 240-252, 254-269, 271-278, 311-326, 331-338, 348-356, 364-370, 375-408, 429-460, 471-482, 484-500, 508-516, 527-536, 539-548, 560-576, 583-605, 643-655, 662-676, 682-687, 691-697, 703-715, 726-734, 737-746, 757-768, 778-789, 791-814, 821-826, 834-854, 890-899, 914-925, 947-954, 959-967, 984-990, 993-1000, 1012-1021, 1039-1044, 1065-1070, 1081-1098, 1136-1142, 1149-1157, 1165-1170, 1175-1186, 1191-1201, 1225-1265, 1276-1285, 1292-1300, 1323-1333, 1351-1361, 1366-1372, 1383-1397, 1404-1412, 1417-1425, 1431-1448, 1468-1473, 1483-1494, 1496-1504 and 1506-1530 of Seq ID No 818; 18-53, 64-93, 95-105, 124-135, 143-148, 155-161, 163-171, 184-198, 238-245, 258-271, 273-284, 287-292, 302-310, 312-320, 322-341, 349-365, 377-403, 407-414, 417-423, 444-453, 455-469, 471-495, 503-511, 536-557, 579-586, 588-609, 619-626, 632-638, 643-649, 656-663, 669-680, 682-688, 699-714, 729-739, 755-761, 768-776, 781-793, 801-815, 821-826, 833-842 and 863-869 of Seq ID No 819; 8-15, 24-40, 51-65, 78-89, 102-111, 117-154, 164-177, 181-192, 198-209, 216-222, 230-237, 241-248, 254-268, 285-293, 298-321, 331-338, 366-373, 384-389, 392-415, 429-439, 441-451, 453-459, 471-486, 489-501 and 524-535 of Seq ID No 820; 10-18, 26-38, 48-54, 60-69, 77-83, 88-95, 119-126, 133-169, 172-185, 193-206, 214-225, 236-250, 269-275, 278-301, 320-329, 336-341, 347-353, 356-369 and 389-396 of Seq ID No 821; 27-32, 37-50, 68-82, 84-108, 134-145, 149-154, 162-170, 172-182, 194-200, 205-224, 232-270, 293-299 and 312-328 of Seq ID No 822; 8-14, 19-45, 65-75, 82-87, 95-105, 135-149, 154-160, 175-184, 205-226, 229-244, 249-256, 284-296, 298-304 and 321-348 of Seq ID No 823; 4-10, 15-24, 26-53, 55-71, 78-83, 90-112, 128-148, 156-163, 165-179, 203-213, 228-239, 250-259, 277-285, 292-314, 322-330, 335-340, 345-360, 363-370, 381-396, 404-409 and 416-427 of Seq ID No 824; 20-32, 37-46, 53-65, 75-83, 86-95, 99-105, 121-133, 139-151, 183-190, 199-205 and 216-227 of Seq ID No 825; 9-25, 29-48, 50-100, 102-126, 131-149, 167-173, 210-217, 224-256, 259-265, 275-292, 295-301, 308-313, 319-335, 337-346, 352-359, 362-382, 393-423, 436-449, 468-476, 481-487, 492-500, 526-534, 537-548, 560-567, 569-579, 590-598, 604-613, 629-636 and 644-656 of Seq ID No 826; 4-18, 25-31, 33-39, 42-53, 64-92, 97-111, 123-129, 134-146, 165-171, 173-190, 192-213, 226-239, 251-273, 283-298, 316-324, 339-345, 350-356, 361-376, 400-408, 418-440, 444-451, 476-481, 503-516, 524-542, 555-563, 581-594, 607-629, 634-641, 647-670, 711-719, 728-738, 755-765, 772-780, 800-815, 822-833, 842-852, 860-865, 874-880, 891-913, 926-938, 941-946, 961-978, 984-990, 1014-1024, 1038-1044, 1052-1092, 1099-1111, 1120-1140, 1153-1168, 1170-1190, 1193-1210, 1221-1233, 1253-1264, 1268-1274, 1283-1289, 1295-1300, 1303-1327, 1338-1351, 1362-1368, 1391-1396, 1410-1416, 1429-1441, 1471-1477, 1483-1513, 1526-1555, 1585-1591, 1596-1630 and 1632-1639 of Seq ID No 827; 10-25, 34-54, 57-67, 77-96, 111-121, 127-139, 151-157, 161-179, 183-198, 201-219, 233-239, 247-252, 268-276, 283-294, 299-309 and 319-324 of Seq ID No 828; 7-15, 20-32, 85-97, 102-109, 117-133, 137-161, 176-184, 186-203, 213-225, 227-251, 258-274, 280-290, 319-325, 335-364, 377-387, 403-410, 412-421, 436-454, 458-475, 478-504, 512-521, 541-567, 601-609, 614-622, 635-651, 657-669, 687-694, 702-708, 717-733, 735-741, 766-786, 788-800, 813-818 and 823-834 of Seq ID No 829; 4-38, 49-69, 75-85, 110-115, 127-134, 167-173, 203-211, 240-245, 258-264, 293-299, 301-316, 348-354, 356-362, 386-391, 405-410, 456-462, 474-483, 494-499, 511-516, 523-528, 533-538, 549-557, 579-585, 587-593, 607-612, 618-625, 627-634, 654-660, 664-670, 682-688, 697-702, 729-736, 749-756, 783-793, 804-812, 817-829, 862-868, 915-920, 944-950, 954-960, 1000-1031, 1044-1056, 1069-1077, 1079-1084, 1097-1118, 1139-1146, 1152-1158, 1165-1176, 1181-1186, 1201-1213, 1257-1263, 1268-1276, 1278-1285, 1354-1360, 1369-1378, 1386-1396, 1439-1446, 1491-1501, 1526-1548 and 1556-1564 of Seq ID No 830; 4-13, 20-34, 47-53, 58-65, 76-82, 89-106, 139-160, 165-182 and 191-205 of Seq ID No 831; 20-40, 42-49, 58-67, 71-80, 95-100, 116-122, 131-142, 145-152, 158-173, 179-186, 196-202, 229-241, 258-270, 289-300, 302-320, 345-351, 370-382, 391-404, 455-464, 504-514 and 516-527 of Seq ID No 832; 5-14, 26-35, 38-45, 54-60, 63-79, 121-127, 137-145, 152-162, 167-173, 175-183, 191-202, 218-228, 238-263, 278-295, 303-316, 320-335, 337-345, 359-365 and 382-400 of Seq ID No 833; 4-17, 31-38, 46-61, 68-73, 76-97, 128-139, 150-156, 166-172, 174-182, 184-212, 219-225, 238-245 and 249-262 of Seq ID No 834; 4-24, 31-42, 45-54, 59-71, 83-92, 108-115, 123-130, 149-156, 202-208 and 224-233 of Seq ID No 835; 4-16, 25-41, 44-52, 60-66, 73-82, 92-101, 108-114, 133-138, 145-155, 177-185 and 194-202 of Seq ID No 836; 4-9, 21-39, 72-78, 82-88, 99-131, 136-143, 151-162, 164-187, 189-204, 208-216, 223-229, 232-240, 246-256, 269-283, 288-299, 311-321 and 328-335 of Seq ID No 837; 26-33, 39-45, 50-62, 79-85, 87-101, 116-131, 142-152, 154-186, 193-199, 201-216, 221-243, 266-272, 281-297, 324-330, 335-342, 345-355, 375-383 and 407-413 of Seq ID No 838; 4-22, 27-36, 60-69, 90-98, 107-113, 117-123, 127-134, 137-151, 154-161, 169-178, 185-192, 202-208, 214-223, 230-239, 245-255, 266-275, 307-317, 323-337, 339-353, 361-379, 385-391, 393-401, 415-422, 424-429, 434-442, 444-449 and 470-480 of Seq ID No 839; 4-22, 29-34, 37-44, 53-80, 98-110, 127-142, 144-156 and 158-165 of Seq ID No 840; 4-12, 14-20, 27-34, 39-47, 51-67, 69-81, 89-97, 105-119, 121-133, 140-149 and 151-161 of Seq ID No 841; 17-24, 34-40, 78-85, 227-233, 294-315, 327-335, 345-351, 354-359, 363-368, 388-403, 405-411, 413-419, 425-434, 462-472, 480-500, 528-536, 542-560, 566-573, 579-589, 593-606, 614-646, 651-658, 663-669, 686-726, 734-747, 754-778, 787-806, 809-825, 827-839 and 876-887 of Seq ID No 842; 9-29, 35-40, 49-63, 69-76, 110-134, 141-147 and 160-169 of Seq ID No 843; 6-31, 33-47, 53-59, 62-78, 93-98, 105-114, 121-130, 136-169, 172-195, 197-208, 223-228, 236-267, 277-283, 295-307, 309-325, 330-339, 345-352, 358-370, 379-391, 419-450, 461-508, 510-519, 521-539, 547-575, 578-587, 589-603, 612-633, 644-657, 666-678, 694-706, 711-717, 728-742, 759-769, 777-784, 800-806, 820-838, 841-848, 851-856, 870-876, 887-895, 908-914, 923-940, 942-953 and 969-988 of Seq ID No 844; 12-39, 41-50, 68-74, 87-97, 113-136, 141-156, 167-180, 190-196, 204-223, 229-235 and 247-278 of Seq ID No 845; 10-16, 25-53 and 64-74 of Seq ID No 846; 5-43, 46-81, 88-96, 137-142, 163-191, 195-203, 210-235, 241-254, 256-276, 280-288, 292-305, 307-313, 317-333, 335-343, 347-353, 357-363, 372-381, 384-389 and 399-409 of Seq ID No 847; 8-14, 22-32, 35-46, 57-75, 83-91, 100-106, 108-114, 125-131, 133-142, 157-175, 186-211, 217-235, 246-361, 367-372, 382-394, 396-405, 414-438, 459-471, 504-510, 513-535 and 538-560 of Seq ID No 848; 8-20, 25-30, 46-62, 67-73, 98-103, 105-114, 119-141, 144-153, 168-178, 181-193, 198-204, 208-227, 236-242, 249-258, 281-288, 291-306, 327-336, 340-361, 368-380, 389-409, 417-426, 428-435, 442-453, 468-486, 488-496, 498-509, 511-523, 540-553, 566-579, 587-603, 629-636 and 677-682 of Seq ID No 849; 9-25, 41-61, 68-75, 81-102, 106-141, 158-165 and 172-191 of Seq ID No 850; 7-38, 43-58, 67-79, 92-99, 103-111, 118-128, 130-139, 152-165, 170-186, 192-223 and 225-251 of Seq ID No 851; 4-16, 21-36, 38-47, 54-64, 92-103, 117-126, 135-155, 157-200, 202-223, 231-239 and 246-262 of Seq ID No 852; 6-38, 44-57, 62-75, 82-96, 104-109, 115-122, 129-136, 147-160, 185-193, 200-206, 230-245, 248-269, 274-282, 289-298, 306-314, 321-335, 344-362, 372-377, 385-394, 422-431, 438-447, 479-505, 529-541, 547-558, 564-571, 573-579, 606-612, 621-632, 638-649, 656-664, 676-683, 695-704, 711-716, 728-734, 736-742, 776-781, 783-791, 809-816, 850-856, 866-872, 889-898, 906-912, 919-926, 944-953, 987-992, 1015-1022, 1024-1038, 1066-1072, 1097-1105, 1113-1119, 1121-1136 and 1147-1154 of Seq ID No 853; 5-14, 27-42, 48-67, 71-83, 85-91, 105-112, 114-135, 139-147, 159-165, 169-185, 188-195, 199-208, 212-221, 231-253, 264-272, 275-282, 290-303, 309-319, 324-331, 340-358, 380-405, 419-425, 438-444, 450-463, 468-477, 497-514, 520-533, 549-556, 568-574, 617-626, 637-643, 661-668, 674-684, 705-713, 718-733 and 735-769 of Seq ID No 854; 5-16, 29-53, 63-71, 74-93, 124-132, 140-192, 199-216, 227-258, 260-268, 272-282, 285-300, 323-331, 353-368, 389-396, 414-421, 429-441, 448-454, 459-467, 474-493, 501-508, 516-575, 593-612, 631-661, 665-693, 715-724, 736-751, 754-765, 771-777, 782-791, 809-820 and 823-853 of Seq ID No 855; 51-70, 81-95, 103-110, 117-123, 130-136, 142-160, 174-180, 199-207, 268-274, 280-296, 347-358, 361-369, 390-396, 401-409, 424-430, 442-448, 455-467, 501-508, 523-533 and 553-560 of Seq ID No 856; 4-10, 35-45, 56-92, 108-116, 127-133, 137-146, 156-186, 194-203, 225-234, 242-251, 272-283, 285-306, 310-315, 322-330, 358-371, 373-379 and 389-396 of Seq ID No 857; 4-32, 38-46, 66-83, 88-95, 110-118, 123-141, 169-180, 200-208, 217-225, 237-245, 247-261, 263-272, 275-282, 291-302, 310-338, 345-353, 360-369, 371-378, 386-394, 398-413, 416-422 and 437-448 of Seq ID No 858; 4-11, 18-26, 31-47, 59-68, 74-92, 98-144, 149-158, 173-180, 200-210, 216-223 and 239-250 of Seq ID No 859; 4-10, 29-56, 93-99, 119-124, 133-140, 159-171, 187-195, 200-214, 221-233, 249-255, 263-271, 285-291 and 310-316 of Seq ID No 860; 29-35, 68-85, 92-99, 107-122, 124-135, 138-144, 146-158, 173-205, 208-227, 232-259, 277-285, 317-324 and 326-333 of Seq ID No 861; 9-18, 21-27, 42-49, 58-75, 85-93, 100-106, 108-122, 125-131, 140-150, 155-162, 165-186, 201-206, 209-214, 220-249, 261-267, 279-289, 292-299, 304-310, 328-338, 343-355, 370-378, 381-389, 393-400, 411-418, 424-436, 438-449, 474-496, 524-531, 556-572, 586-592, 606-617, 623-629, 648-657, 659-676, 696-702, 709-717, 735-755, 763-775, 788-809 and 835-843 of Seq ID No 862; 20-32, 52-63, 75-88, 96-101, 116-154, 164-173, 187-199, 202-240, 253-279, 285-298, 305-318 and 324-339 of Seq ID No 863; 4-22, 24-29, 36-43, 63-75, 90-96, 118-128, 137-145, 168-182, 200-210, 212-221, 242-250, 289-316, 318-323, 327-339, 381-387, 401-411, 424-434, 443-449, 453-465, 485-498, 500-508, 510-515, 521-528, 538-545, 554-560, 574-606, 619-627, 645-658 and 681-688 of Seq ID No 864; 8-18, 45-50, 52-62, 76-82, 84-107, 109-116, 130-137, 141-150, 152-158, 164-170, 175-186 and 188-196 of Seq ID No 865; 11-19, 24-34, 41-48, 55-63, 68-81, 85-91, 100-106, 111-120, 131-138, 144-161, 168-176, 192-203, 213-225, 227-234, 236-243, 255-262, 265-274, 282-290, 296-301, 309-316, 349-359, 368-377, 384-390, 398-412, 417-433, 439-448, 467-475, 481-486, 501-508, 510-517, 521-532 and 538-545 of Seq ID No 866; 442, 48-59, 74-88, 92-119, 121-149, 163-180, 185-195 and 199-209 of Seq ID No 867; 5-26, 60-76, 104-114, 119-128, 136-141, 156-167, 186-198, 218-237, 260-267, 275-290, 309-318 and 328-335 of Seq ID No 868; 5-17, 23-48, 60-73, 75-82, 98-108, 110-128, 146-160, 168-180, 191-213, 229-237, 240-252, 269-277 and 305-313 of Seq ID No 869; 8-28, 53-62, 70-78, 81-89, 97-115, 125-148, 155-168, 174-186, 196-202, 207-214, 220-238, 241-256, 258-267, 284-290, 297-306, 312-317, 322-327, 330-344, 352-358, 367-385, 387-395, 400-414, 422-430, 438-455, 458-466, 491-507, 539-544, 561-566, 571-577, 598-604, 617-623 and 640-647 of Seq ID No 870; 7-14, 24-32, 56-72, 95-100, 108-114, 123-138, 143-153, 203-221, 224-230, 260-269, 290-297, 302-308, 321-355, 364-370, 398-427, 434-439, 446-473, 505-510, 512-518, 536-546, 573-587, 589-595, 629-636, 683-709, 728-734, 778-786, 795-802, 825-830, 911-934, 944-956, 960-970, 977-985, 987-993, 1009-1015, 1027-1035, 1047-1052 and 1058-1065 of Seq ID No 871; 4-16, 21-36, 38-47, 54-64, 92-103, 117-126, 135-155, 157-200, 202-223, 231-239 and 246-262 of Seq ID No 872; 4-10, 35-45, 56-92, 108-116, 127-133, 137-146, 156-186, 194-203, 225-234, 242-251, 272-283, 285-306, 310-315, 322-330, 358-371, 373-379 and 389-396 of Seq ID No 873; 4-40, 47-59, 62-75, 81-87 and 107-113 of Seq ID No 874; 6-13, 21-31, 44-54, 64-70 and 77-84 of Seq ID No 875; 17-24, 26-41 and 50-55 of Seq ID No 876; 24-32 of Seq ID No 877; 15-28 and 36-44 of Seq ID No 878; 4-25, 28-34, 37-43, 45-52, 59-69, 85-91 and 104-114 of Seq ID No 879; 13-39, 51-63, 80-101, 141-149, 165-176 and 191-210 of Seq ID No 880; 6-19 of Seq ID No 881; 4-19, 23-42, 57-66, 98-114, 121-127, 148-155, 164-181, 194-204, 217-222, 248-258, 266-285, 288-296, 309-329, 331-342, 344-353 and 361-378 of Seq ID No 882; 4-14 and 26-32 of Seq ID No 883; 10-18, 29-62, 72-91, 106-120, 147-154, 185-195, 203-210, 220-238, 262-267, 275-285, 315-365, 376-388, 390-399 and 414-423 of Seq ID No 884;8-17, 43-49, 63-70, 79-86 and 89-100 of Seq ID No 885; 4-19, 21-31 and 54-68 of Seq ID No 886; 4-10 and 26-31 of Seq ID No 887; 4-12, 21-30 and 37-51 of Seq ID No 888; 4-11 and 20-46 of Seq ID No 889; 4-10 and 23-28 of Seq ID No 890; 4-28 and 32-50 of Seq ID No 891; 4-25 of Seq ID No 892; 35-53 of Seq ID No 893; 14-20, 48-56, 63-68 and 79-87 of Seq ID No 894; 34-40 and 50-62 of Seq ID No 895; 23-34 and 59-72 of Seq ID No 896; 4-16, 28-39, 62-68 and 85-97 of Seq ID No 897; 10-20, 23-31, 35-42 and 48-62 of Seq ID No 898; 4-44, 46-52, 54-64 and 70-85 of Seq ID No 899; 4-12 and 24-40 of Seq ID No 900; 10-22, 24-46, 52-57 and 74-81 of Seq ID No 901; 10-20, 27-40 and 48-57 of Seq ID No 902; 24-37 of Seq ID No 903; 20-29, 39-45, 53-65 and 67-85 of Seq ID No 904; 6-22, 25-41, 43-66, 74-84, 87-94 and 107-113 of Seq ID No 905; 4-12, 17-23 and 39-62 of Seq ID No 906; 4-13, 21-45 and 51-70 of Seq ID No 907; 15-25 and 33-52 of Seq ID No 908; 8-19, 44-53, 60-70, 78-85 and 97-107 of Seq ID No 909; 13-23, 31-40, 59-66, 8490 and 96-110 of Seq ID No 910; 4-25, 37-48, 56-71, 83-97, 112-132, 140-150 and 152-157 of Seq ID No 911; 4-17, 19-26, 41-49, 63-87, 92-99 and 113-131 of Seq ID No 912; 4-10 and 17-23 of Seq ID No 913; 4-19, 26-35, 43-50 and 61-72 of Seq ID No 914; 5-10, 31-38, 42-70 and 84-99 of Seq ID. No 915; 12-26 and 48-55 of Seq ID No 916; 4-10, 13-63, 69-81, 83-105 and 143-150 of Seq ID No. 917; 4-12, 18-29 and 49-68 of Seq ID No 918; 19-25, 27-33, 43-84, 86-92, 111-118, 125-136, 138-147, 158-165 and 181-192 of Seq ID No 919; 20-29, 50-56, 63-85, 89-98 and 120-128 of Seq ID No 920; 4-11 and 41-47 of Seq ID No 921; 14-27 and 50-62 of Seq ID No 922; 10-19, 35-41 and 43-51 of Seq ID No 923; 17-40, 47-66 and 70-78 of Seq ID No 924; 549-576 and 595-622 of Seq ID No 1007; 592-616 of Seq ID No 1031; 9-22 of Seq ID No 1032; 896-923 of Seq ID No 317; 178-204, 222-248, 244-269 and 265-296 of Seq ID No 320; 180-209 and 205-233 of Seq ID No 321; 242-270 and 266-294 of Seq ID No 322; 718-740, 328; 197-229 and 225-256 of Seq ID No 345; 42-61 of Seq ID No 359; 34-53 of Seq ID No 425; 1522-1552 of Seq ID No 431; 415-440 of Seq ID No 462; 32-51 of Seq ID No 463; 342-369 and 457-484 of Seq ID No 466; 554-582, 578-606 and 632-659 of Seq ID No 467; 12-33 and 27-48 of Seq ID No 469; 52-81, 77-105 and 101-129 of Seq ID No 471; 76-102 of Seq ID No 472; 48-77 of Seq ID No 473; 6-35 of Seq ID No 474; 189-218 and 214-244 of Seq ID No 456; 122-149 of Seq ID No 459; 106-136 of Seq ID No 460; 204-224 of Seq ID No 477; 273-297 of Seq ID No 301; 70-88 of Seq ID No 302; 134-147 of Seq ID No 303; 290-317 and 367-388 of Seq ID No 304; 240-265 of Seq ID No 305; 796-824 of Seq ID No 306; 83-110 and 106-133 of Seq ID No 308; 120-148 of Seq ID No 309; 71-106 of Seq ID No 310; 278-305 of Seq ID No 312; 115-137 of Seq ID No 313; 378-400 of Seq ID No 315; 111-137 of Seq ID No 316;3-19 of Seq ID No 318; 103-119 of Seq ID No 323; 89-116, 112-139 and 135-162 of Seq ID No 326; 259-268 of Seq ID No 390; 2-19 of Seq ID No 501; 83-114 of Seq ID No 488; 15-33 of Seq ID No 496; 52-84 and 70-93 of Seq ID No 498; 28-54 of Seq ID No 502; 34-66 of Seq ID No 514; 4-32 and 28-56 of Seq ID No 528; 21-47 of Seq ID No 513; 32-57 of Seq ID No 490; 96-115 of Seq ID No 542; 161-176 of Seq ID No 543; 58-89, 85-116 and 112-143 of Seq ID No 557; 97-115 of Seq ID No 559; 84-106 of Seq ID No 565; 3-19 of Seq ID No 572; 38-63 of Seq ID No 575; 33-64, 60-91 and 87-119 of Seq ID No 580; 93-123 of Seq ID No 558; 16-34 of Seq ID No 563; 61-92 and 88-120 of Seq ID No 593; 48-76, 72-100, 96-124 and 120-147 of Seq ID No 568; 111-146 of Seq ID No 569; 26-50, 46-71 and 67-92 of Seq ID No 570; 11-46 of Seq ID No 573; 92-127 of Seq ID No 574; 46-78, 74-106 and 102-133 of Seq ID No 582; 94-124 of Seq ID No 584; 2-29 of Seq ID No 585; 10-43 of Seq ID No 597; 24-51, 47-74 and 70-98 of Seq ID No 600; 15-42 and 38-58 of Seq ID No 551; 259-268 of Seq ID No 390; 66-94 of Seq ID No 598; 17-43 of Seq ID No 594; 178-198 of Seq ID No 375; 441-473, 469-501 and 497-531 of Seq ID No 385; 105-127 of Seq ID No 387; 8-35, 36-63 and 64-91 of Seq ID No 388; 216-231 of Seq ID No 391; 203-222 of Seq ID No 395; 180-210, 206-236 and 232-261 of Seq ID No 404; 6-28 of Seq ID No 405; 224-238 of Seq ID No 408; 274-291 of Seq ID No 411; 134-148 of Seq ID No 413; 133-153 of Seq ID No 414; 258-284 of Seq ID No 416; 8-33, 28-53 and 49-74 of Seq ID No 419; 103-127, 123-147, 143-166 and 162-185 of Seq ID No 420; 179-206 of Seq ID No 421; 15-27 of Seq ID No 434; 240-261 of Seq ID No 435; 386-402 of Seq ID No 437; 57-74 of Seq ID No 438; 343-375, 371-403 and 399-432 of Seq ID No 448; 369-390 of Seq ID No 450; 276-292 of Seq ID No 452; 196-227, 223-254, 250-280, 284-311, 307-334, 330-356 and 352-378 of Seq ID No 453; 253-271 of Seq ID No 454; 419-432 of Seq ID No 455; 24-51 of Seq ID No 370; 259-268 of Seq ID No 390; 23-48 of Seq ID No 371; 179-198 of Seq ID No 372; 431-455 of Seq ID No 373; 319-349, 345-374 and 370-399 of Seq ID No 374; 455-479 and 475-498 of Seq ID No 376; 653-675 of Seq ID No 377; 75-108 of Seq ID No 378; 1362-1388 of Seq ID No 379; 8-36, 32-60 and 56-84 of Seq ID No 380; 1-25 of Seq ID No 381; 65-88 of Seq ID No 382; 102-134 of Seq ID No 383; 104-131 of Seq ID No 384; 206-231 of Seq ID No 386; 5-23 of Seq ID No 389; 510-536 and 546-577 of Seq ID No 390; 300-326 and 1320-1353 of Seq ID No 392; 205-240 of Seq ID No 393; 971-1003 of Seq ID No 394; 455-483 and 1232-1263 of Seq ID No 396; 12-40 of Seq ID No 397; 3-32 of Seq ID No 398; 68-103 of Seq ID No 399; 188-222 of Seq ID No 400; 25-55 of Seq ID No 401; 98-130 of Seq ID No 402; 211-236 of Seq ID No 403; 362-392 of Seq ID No 406; 365-389 of Seq ID No 407; 60-81 of Seq ID No 409; 69-104 of Seq ID No 410; 124-152 and 148-177 of Seq ID No 412; 74-102 of Seq ID No 415; 1019-1051, 1162-1190, 1186-1214, 1210-1238 and 1234-1261 of Seq ID No 417; 692-724 of Seq ID No 423; 259-268 of Seq ID No 390; 15-41, 37-62 and 58-83 of Seq ID No 418; 59-87, 83-110 and 111-140 of Seq ID No 422; 174-206 of Seq ID No 424; 96-119 of Seq ID No 426; 128-152 of Seq ID No 427; 104-130 of Seq ID No 428; 542-568 of Seq ID No 429; 107-129 of Seq ID No 430; 112-137 of Seq ID No 432; 52-80 of Seq ID No 433; 19-45 of Seq ID No 436; 1113-1144 of Seq ID No 441; 94-119, 110-135 and 136-160 of Seq ID No 439; 89-115 of Seq ID No 442; 474-496 and 492-515 of Seq ID No 443; 14-40 of Seq ID No 444; 422-448 of Seq ID No 445; 294-333 of Seq ID No 447; 113-140 of Seq ID No 449; 8-36, 32-61 and 57-86 of Seq ID No 451; 1-28, 120-147 and 143-170 of Seq ID No 440; -14-11 of Seq ID No 446; 96-124 of Seq ID No 1042; 550-575, 571-596 and 592-616 of Seq ID No1031; 144-170 of Seq ID No1046; 79-100 of Seq ID No1048; 73-96 of Seq ID No1050; 21-43 of Seq ID No1051; 18-42 of Seq ID No1052; 16-50 of Seq ID No1053; 7-25 of Seq ID No1055; 8-44 of Seq ID No1056; 22-51 of Seq ID No1057; 2-25 of Seq ID No1059; 9-44 of Seq ID No1060; 6-34, 30-58, 54-82 and 78-105 of Seq ID No1061; 9-32 of Seq ID No1062; 23-46 and 42-65 of Seq ID No1063; 1-25 of Seq ID No1064; 17-43 of Seq ID No1065; 11-38 of Seq ID No1067; 44-72, 68-95 and 91-118 of Seq ID No1009; 163-187 of Seq ID No1043; 586-612 of Seq ID No1011; 259-284, 283-313, 309-339 and 335-365 of Seq ID No1012; 311-333 of Seq ID No1016; 245-269, 474-504, 500-530 and 526-555 of Seq ID No1018; 270-302, 298-330 and 326-358 of Seq ID No1021; 58-86, 82-109, 105-133, 325-353, 349-378 and 374-403 of Seq ID No1023; 9-33 of Seq ID No1032; 96-124 of Seq ID No1042; 447-475, 471-498 and 494-521 of Seq ID No 1022; 285-316, 312-343, 339-371, 367-399, 515-541, 537-564 and 560-586 of Seq ID No 1027; 149-175, 171-197 and 193-217 of Seq ID No 997; 43-73, 69-99, 95-124, 163-189, 185-210, 206-231, 227-252, 248-273, 269-294, 313-340, 336-362, 358-384, 456-481, 477-502 and 498-523 of Seq ID No 998; 136-163, 159-186, 193-217, 253-279, 275-301, 447-474, 470-496, 492-518, 519-547 and 543-572 of Seq ID No 999; 31-58, 54-80, 76-102, 166-196, 222-246, 242-266, 284-310, 306-332 and; 328-354 of Seq ID No 1000; 96-124 of Seq ID No 1042; 35-58, 54-77 and; 73-96 of Seq ID No 1001; 200-229, 225-253, 249-277, 495-522, 518-544 and 540-566 of Seq ID No 1002; 238-267, 268-300 and; 301-330 of Seq ID No 1003; 6-36, 32-62, 58-87, 210-237, 233-260, 256-282, 496-522, 518-543 and; 539-564 of Seq ID No 1004; 26-53, 468-501, 497-530, 49-76, 72-98, 235-268, 253-283, 279-309, 305-334, 425-455 and; 439-472 of Seq ID No 1005; 7-40 and; 36-69 of Seq ID No 1006; 11-38, 34-61, 57-84, 126-153, 149-176, 172-198, 265-290, 286-311 and 307-331 of Seq ID No 1045; 115-137 of Seq ID No 313; 11-35 of Seq ID No 1307; 1-25 of Seq ID No 1312; 8-32 of Seq ID No 1313; 5-29 of Seq ID No 1317; 10-34 of Seq ID No 1318; 82-106 of Seq ID No 1320; 3-27 of Seq ID No 1321; 9-33 of Seq ID No 1322;1-25 of Seq ID No 1323; 16-40 of Seq ID No 1324; 4-35 and 31-61 of Seq ID No 1325; 67-91 of Seq ID No 1326; 25-52 of Seq ID No 1327; 20-44 of Seq ID No 1329; 1-25 of Seq ID No 1330; 5-32 and 28-54 of Seq ID No 1331; 14-38 of Seq ID No 1332; 23-47 of Seq ID No 1333; 8-32 of Seq ID No 1334; 18-42 of Seq ID No 1335; 3-27 of Seq ID No 1336; 181-203 of Seq ID No 1203; 450-474 of Seq ID No 1204; 200-224, 220-245 and 241-266 of Seq ID No 1205; 50-79 and 75-104 of Seq ID No 1206; 195-219 of Seq ID No 1207; 26-53 and 49-77 of Seq ID No 1208; 107-136 and 132-162 of Seq ID No 1209; 197-221 of Seq ID No 1210; 10-34 of Seq ID No 1211; 48-80 and 76-107 of Seq ID No 1212; 159-191, 187-218, 434-464 and 460-489 of Seq ID No 1213; 66-90 of Seq ID No 1214; 51-75 and 666-690 of Seq ID No 1215; 99-123 of Seq ID No 1216; 251-275 of Seq ID No 1217; 115-137 of Seq ID No 313; 102-129 and 125-151 of Seq ID No 1218; 30-54 of Seq ID No 1219; 138-162 of Seq ID No 1220; 79-103 of Seq ID No 1221; 101-125 of Seq ID No 1222; 126-154, 150-178 and 174-202 of Seq ID No 1223; 322-346 of Seq ID No 1224; 418-442 of Seq ID No 1225; 126-156, 152-182, 178-208, 436-465, 461-489 and 485-513 of Seq ID No 1226; 42-75, 71-104, 134-161, 157-184, 181-209 and 205-233 of Seq ID No 1227; 131-155 and 262-286 of Seq ID No 1228; 43-69 and 65-90 of Seq ID No 1229; 468-492 of Seq ID No 1230; 90-115, 111-135, 131-155 and 151-175 of Seq ID No 1231; 495-522 and 518-545 of Seq ID No 1232; 45-72 and 68-95 of Seq ID No 1233; 94-121 and 117-144 of Seq ID No 1234; 8-32 and 337-361 of Seq ID No 1235; 943-973 and 969-1000 of Seq ID No 1236; 191-215 of Seq ID No 1237; 70-97 and 93-120 of Seq ID No 1238; 800-824, 820-844 and 840-864 of Seq ID No 1242; 115-137 of Seq ID No 313; 341-363, 359-381, 376-402, 398-424 and 420-445 of Seq ID No 1241; 186-216 of Seq ID No 1243; 103-127 of Seq ID No 1244; 19-50 and 46-77 of Seq ID No 1245; 80-107, 103-129 and 125-151 of Seq ID No 1246; 406-430 of Seq ID No 1247; 795-827 and 823-855 of Seq ID No 1248; 404-428 of Seq ID No 1249; 66-90, 86-110 and 106-130 of Seq ID No 1250; 107-131 of Seq ID No 1251; 524-548 of Seq ID No 1252; 193-217, 293-323, 319-349, 345-375 and 371-400 of Seq ID No 1253; 481-505 of Seq ID No 1254; 33-58 and 54-78 of Seq ID No 1255; 281-308, 304-331 and 327-354 of Seq ID No 1256; 321-352 of Seq ID No 1257; 174-198 of Seq ID No 1258; 248-273, 269-293 and 289-313 of Seq ID No 1259; 249-281 and 277-310 of Seq ID No 1260; 31-55 of Seq ID No 1261; 415-439, 540-566 and 562-589 of Seq ID No 1262; 213-237 of Seq ID No 1263; 243-267 of Seq ID No 1264; 545-583 of Seq ID No 1265; 73-97 and 240-264 of Seq ID No 1267; 115-137 of Seq ID No 313; 48-73, 69-93 and 89-113 of Seq ID No 1266; 9-33 of Seq ID No 1269; 375-399 of Seq ID No 1270; 335-359, 461-489, 485-513 and 509-536 of Seq ID No 1271; 30-62 and 58-90 of Seq ID No 1272; 581-605 of Seq ID No 1273; 101-131 and 127-157 of Seq ID No 1274; 317-349 and 345-377 of Seq ID No 1275; 534-558 of Seq ID No 1276; 108-133, 234-261, 257-284, 280-307 and 303-330 of Seq ID No 1277; 2-27, 23-48, 44-68, 82-106, 104-128, 152-176 and 373-397 of Seq ID No 1278; 324-348 of Seq ID No 1279; 196-223 and 219-246 of Seq ID No 1280; 263-294 and 291-323 of Seq ID No 1281; 183-207 of Seq ID No 1282; 176-200 of Seq ID No 1283; 297-321 of Seq ID No 1284; 91-115 of Seq ID No 1285; 11-41, 37-67 and 63-92 of Seq ID No 1286; 157-190 and 186-219 of Seq ID No 1287; 240-264 of Seq ID No 1288; 51-75 of Seq ID No 1289; 31-55 of Seq ID No 1290; 166-190 of Seq ID No 1291; 10-34 of Seq ID No 1293; 115-137 of Seq ID No 313; 554-579, 575-600, 596-621 and 617-642 of Seq ID No 1292; 200-226, 222-248 and 244 269 of Seq ID No 1295; 4-28 of Seq ID No 1296; 476-503 of Seq ID No 1297; 370-394 of Seq ID No 1298; 129-153 of Seq ID No 1299; 51-75 of Seq ID No 1301; 88-112 of Seq ID No 1302; 670-694 and 848-872 of Seq ID No 1303; 164-196, 703-731, 764-796, 792-823, 192-223, 219-250, 438-463, 459-483, 479-503, 629-658, 654-683 and 679-707 of Seq ID No 1306; 5-36 of Seq ID No 461; 32-51 of Seq ID No 463; 16-46 of Seq ID No 468; 12-33 and 27-48 of Seq ID No 469; 10-39 of Seq ID No 470; 27-56, 52-81, 77-105 and 101-129 of Seq ID No 471; 30-57, 53-80 and 76-102 of Seq ID No 472; 48-77 of Seq ID No 473; 6-35 of Seq ID No 474; 233-257, 253-277, 273-297 and; 293-317 of Seq ID No 301; 290-317 and; 367-388 of Seq ID No 303; 240-265 of Seq ID No 305; 796-824 of Seq ID No 306; 624-653 of Seq ID No 307; 36-64, 60-87, 83-110 and; 106-133 of Seq ID No 308; 120-148 of Seq ID No 309; 71-106 of Seq ID No 310; 741-764 and; 760-783 of Seq ID No 311; 450-483 of Seq ID No 314; 111-137 of Seq ID No 316; 896-923 of Seq ID No 317; 565-595 of Seq ID No 319; 178-204, 200-226, 222-248, 244-269 and 265-296 of Seq ID No 320; 1343-1369, 180-209, 205-233 and; 229-258 of Seq ID No 321; 242-270, 266-294 and; 290-318 of Seq ID No 322; 259-268 of Seq ID No 360; 278-305 of Seq ID No 312; 115-137 of Seq ID No 313; 89-116, 112-139 and; 135-162 of Seq ID No 326; 76-97 and; 93-114 of Seq ID No 329; 39-66, 62-88 and; 84-110 of Seq ID No 331; 244-274 of Seq ID No 333; 548-572 of Seq ID No 335; 165-200 of Seq ID No 336; 1-35 of Seq ID No 338; 662-695 of Seq ID No 339; 232-256 and; 252-283 of Seq ID No 342; 588-620 of Seq ID No 343; 197-229, 225-256 and; 252-283 of Seq ID No 345; 212-244, 240-272 and; 395-429 of Seq ID No 347; 209-230 of Seq ID No 348; 78-106 and 102-130 of Seq ID No 349; 118-142 of Seq ID No 350; 105-133, 129-156 and 152-180 of Seq ID No 351; 147-175 of Seq ID No 352; 16-44 of Seq ID No 353; 102-131 of Seq ID No 354; 328-355 of Seq ID No 355; 436-465 of Seq ID No 357; 139-166 of Seq ID No 363; 373-401 of Seq ID No 365; 114-148 of Seq ID No 367; 23-48, 44-70 and 71-99 of Seq ID No 369; 34-53 of Seq ID No 425; 164-193, 189-218 and 214-244 of Seq ID No 456; 1522-1552 of Seq ID No 431; 17-49 of Seq ID No 475; 122-149 of Seq ID No 459; 106-136 of Seq ID No 460; 26-51, 47-72 and 68-92 of Seq ID No 476; 187-208 and 204-224 of Seq ID No 477; 654-682 of Seq ID No 361; 944-970, 966-992 and 988-1015 of Seq ID No 364; 242-256 and 60-88 of Seq ID No 457; 96-124 of Seq ID No 1042; 550-575, 571-596 and 592-616 of Seq ID No 1031; 9-22 of Seq ID No 1032; 139-163, 159-183, 179-203, 199-220, 549-576, 572-599 and 595-622 of Seq ID No 1007; 70-88 of Seq ID No 302;134-147 of Seq ID No 303; 378-400 of Seq ID No 315; 3-19 of Seq ID No 318; 103-119 of Seq ID No 323; 266-292 of Seq ID No 324; 179-193 of Seq ID No 325; 282-296 of Seq ID No 327; 718-740 of Seq ID No 328; 1-29, 30-58 and 59-87 of Seq ID No 330; 54-69 of Seq ID No 332; 33-56 of Seq ID No 334; 71-84 of Seq ID No 340; 60-70 of Seq ID No 341; 17-41 of Seq ID No 344; 110-127 of Seq ID No 346; 101-122 of Seq ID No 356; 408-427 of Seq ID No 358; 42-61 of Seq ID No 359; 40-59 of Seq ID No 362; 178-193 of Seq ID No 366; 518-545 and 541-568 of Seq ID No 368; 564-591 of Seq ID No 368; 415-440, 436-461, 457-482 and 478-503 of Seq ID No 462; 1-29, 53-80, 76-103 and 99-126 of Seq ID No 464; 342-366, 362-386, 382-406 and 402-428 of Seq ID No 465; 342-369, 365-392, 388-415, 411-438, 434-461 and 457-484 of Seq ID No 466; 530-558, 554-582, 578-606 and 632-659 of Seq ID No 467; 34-42, 56-87, 95-133, 136-146, 157-213, 219-235, 246-282, 313-333, 358-394 and 196-215 of Seq ID No 1365; 67-74, 88-94, 112-118, 127-138, 155-169, 171-180, 183-190, 196-205, 243-249, 260-271, 308-344, 346-373, 381-414, 416-457, 473-513, 515-524, 528-535, 539-544, 556-566, 572-580, 585-590 and 27-129 of Seq ID No 1366; 4-16, 21-36, 38-47, 54-64, 92-103, 117-126, 135-155, 157-200, 202-223, 231-239, 246-262 and 128-153 of Seq ID No 1367; 4-10, 16-30 and 11-43 of Seq ID No 1369; 7-22, 52-77, 83-93, 101-111, 125-136, 139-157, 212-221, 231-239, 241-247, 264-273, 275-294, 329-336, 349-357, 365-379, 389-405, 419-434, 439-445, 456-462, 467-481, 493-506, 508-516, 522-545, 547-556, 566-583, 611-627, 655-670, 678-693, 717-724, 734-748, 756-766, 772-790, 797-808, 815-820, 825-831, 833-838, 851-868, 891-917, 919-926, 933-940, 944-953 and 246-271 of Seq ID No 1370; 4-12, 14-31, 42-59, 61-69, 73-83, 96-103, 140-149, 153-165, 180-187, 199-208, 222-230, 232-240, 248-254, 256-270, 274-283, 289-299, 302-317, 322-328, 332-345, 351-365, 387-396, 419-432, 441-447, 453-466, 487-505, 508-524, 560-571, 580-590, 592-605, 625-633, 639-647, 652-658, 671-679, 722-728 and 660-694 of Seq ID No 1371; 4-13, 26-39, 53-59, 68-74, 88-95, 102-119, 125-132, 136-150, 153-162, 165-175, 188-228, 238-245, 268-283, 285-307, 317-324, 326-343, 350-359 and 79-101 of Seq ID No 1372; 10-37, 55-68, 71-78, 92-98, 115-122, 131-138, 149-158, 163-170, 172-189, 212-219, 239-257, 259-271, 289-302, 304-320, 322-340, 359-366, 373-384, 400-412, 444-453, 460-474, 485-527 and 187-224 of Seq ID No 1373; 13-21, 27-36, 41-50, 55-64, 66-72, 74-90, 103-112, 127-136, 153-164, 166-186, 193-219, 224-242, 260-273, 278-294, 298-306, 328-334 and 142-171 of Seq ID No 1374; 16-29, 31-41, 49-61, 63-81, 86-92, 107-114, 122-135, 155-177, 195-211, 245-252, 264-270, 273-279, 297-322, 327-334, 339-348, 371-378, 380-389, 402-408, 414-421, 424-430, 452-459, 481-488, 500-506, 543-556, 567-573, 588-594, 608-615, 628-633, 668-675, 683-689, 700-706, 735-750, 793-802, 816-822, 841-846, 848-855, 858-864, 894-913, 921-929, 941-948, 974-990, 993-1005, 1053-1068, 1096-1110, 1117-1123, 1126-1145, 1149-1168, 1191-1203, 1219-1225, 1239-1248, 1253-1265, 1297-1309, 1356-1370, 1373-1379, 1388-1395 and 372-393 of Seq ID No 1375; 5-15, 29-37, 39-46, 51-60, 65-71, 73-97, 105-131, 137-152, 154-161, 173-185, 193-210, 214-222, 224-232, 241-255, 266-274, 277-289, 291-303, 307-338, 345-352, 358-371, 389-395, 402-422, 433-440, 444-465, 471-478, 498-513, 524-536, 542-558, 561-576, 584-602, 604-623, 631-639, 643-658, 667-683, 689-716 and 315-397 of Seq ID No 1376; 7-25, 30-37, 39-60, 69-86 and 75-89 of Seq ID No 1377; 16-30 of Seq ID No 1378; 8-27 of Seq ID No 1379; 4-22, 29-48, 50-58, 62-69, 71-78 and 6-36 of Seq ID No 1380; 4-23 and 3-24 of Seq ID No 1381; 4-12, 30-66 and 11-33 of Seq ID No 1382; 4-9, 40-64, 68-82 and 54-72 of Seq ID No 1383; 4-42 and 11-39 of Seq ID No 1384; 15-24, 77-88, 90-103, 116-123, 134-144 and 23-33 of Seq ID No 1385; 17-25, 47-56, 68-79 and 64-74 of Seq ID No 1386; 4-12, 14-21, 27-33 and 7-25 of Seq ID No 1387; 4-10, 25-37 and 5-23 of Seq ID No 1388; 4-9, 14-29, 38-44, 49-67, 69-97, 118-128, 135-146, 151-157, 159-172, 196-203, 227-241, 253-259, 262-271, 284-295, 299-309, 343-353, 356-362, 392-406, 410-416, 429-441, 463-478, 503-509 and 495-511 of Seq ID No 1389; 4-12, 24-33, 39-51, 57-63, 78-87 and 32-56 of Seq ID No 1390; 8-15, 29-40, 48-54 and 33-56 of Seq ID No 1391; 12-61 and 35-61 of Seq ID No 1392; 8-27 of Seq ID No 1393 and fragments comprising at least 6, preferably more than 8, especially more than 10 aa of said sequences. All these fragments individually and each independently form a preferred selected aspect of the present invention.

All linear hyperimmune serum reactive fragments of a particular antigen may be identified by analysing the entire sequence of the protein antigen by a set of peptides overlapping by 1 amino acid with a length of at least 10 amino acids. Subsequently, non-linear epitopes can be identified by analysis of the protein antigen with hyperimmune sera using the expressed full-length protein or domain polypeptides thereof. Assuming that a distinct domain of a protein is sufficient to form the 3D structure independent from the native protein, the analysis of the respective recombinant or synthetically produced domain polypeptide with hyperimmune serum would allow the identification of conformational epitopes within the individual domains of multi-domain proteins. For those antigens where a domain possesses linear as well as conformational epitopes, competition experiments with peptides corresponding to the linear epitopes may be used to confirm the presence of conformational epitopes.

It will be appreciated that the invention also relates to, among others, nucleic acid molecules encoding the aforementioned fragments, nucleic acid molecules that hybridise to nucleic acid molecules encoding the fragments, particularly those that hybridise under stringent conditions, and nucleic acid molecules, such as PCR primers, for amplifying nucleic acid molecules that encode the fragments. In these regards, preferred nucleic acid molecules are those that correspond to the preferred fragments, as discussed above.

The present invention also relates to vectors, which comprise a nucleic acid molecule or nucleic acid molecules of the present invention, host cells which are genetically engineered with vectors of the invention and the production of hyperimmune serum reactive antigens and fragments thereof by recombinant techniques.

A great variety of expression vectors can be used to express a hyperimmune serum reactive antigen or fragment thereof according to the present invention. Generally, any vector suitable to maintain, propagate or express nucleic adds to express a polypeptide in a host may be used for expression in this regard. In accordance with this aspect of the invention the vector may be, for example, a plasmid vector, a single or double-stranded phage vector, a single or double-stranded RNA or DNA viral vector. Starting plasmids disposed herein are either commercially available, publicly available, or cat be constructed from available plasmids by routine application of well-known, published procedures. Preferred among vectors, in certain respects, are those for expression of nucleic acid molecules and hyperimmune serum reactive antigens or fragments thereof of the present invention. Nucleic acid constructs in host cells can be used in a conventional manner to produce the gene product encoded by the recombinant sequence. Alternatively, the hyperimmune serum reactive antigens and fragments thereof of the invention can be synthetically produced by conventional peptide synthesizers. Mature proteins can be expressed in mammalian cells, yeast, bacteria, or other cells under the control of appropriate promoters. Cell-free translation systems can also be employed to produce such proteins using RNAs derived from the DNA construct of the present invention.

Host cells can be genetically engineered to incorporate nucleic acid molecules and express nucleic acid molecules of the present invention Representative examples of appropriate hosts include bacterial cells, such as streptococci, staphylococci, E. coli, Streptomyces and Bacillus subtillis cells; fungal cells, such as yeast cells and Aspergillus cells; insect cells such as Drosophila S2 and Spodoptera Sf9 cells; animal cells such as CHO, COS, Hela, C127, 3T3, BHK, 293 and Bowes melanoma cells; and plant cells.

The invention also provides a process for producing an enteroaggregative E. coli, enterotoxigenic E. coli, S. flexneri and/or C. jejuni hyperimmune serum reactive antigen and a fragment thereof comprising expressing from the host cell a hyperimmune serum reactive antigen or fragment thereof encoded by the nucleic add molecules provided by the present invention. The invention further provides a process for producing a cell, which expresses an enteroaggregative E. coli, enterotoxigenic E. coli, S. flexneri and/or C. jejuni hyperimmune serum reactive antigen or a fragment thereof comprising transforming or transfecting a suitable host cell with the vector according to the present invention such that the transformed or transfected cell expresses the polypeptide encoded by the nucleic acid contained in the vector.

The polypeptide may be expressed in a modified form, such as a fusion protein, and may include not only secretion signals but also additional heterologous functional regions. Thus, for instance, a region of additional amino adds, particularly charged amino acids, may be added to the N— or C-terminus of the polypeptide to improve stability and persistence in the host cell, during purification or during subsequent handling and storage. Also, regions may be added to the polypeptide to facilitate purification. Such regions may be removed prior to final preparation of the polypeptide. The addition of peptide moieties to polypeptides to engender secretion or excretion, to improve stability or to facilitate purification, among others, are familiar and routine techniques in the art. A preferred fusion protein comprises a heterologous region from immunoglobulin that is useful to solubilize or purify polypeptides. For example, BP-A-O 464 533 (Canadian counterpart 2045869) discloses fusion proteins comprising various portions of constant region of immunoglobin molecules together with another protein or part thereof In drug discovery, for example, proteins have been fused with antibody Fc portions for the purpose of high-throughout screening assays to identify antagonists. See for example, {Bennett, D. et al., 1995} and {Johanson, K. et al., 1995}.

The enteroaggregative E. coli, enterotoxigenic E. coli, S. flexneri or C. jejuni hyperimmune serum reactive antigen or a fragment thereof can be recovered and purified from recombinant cell cultures by well-known methods including ammonium sulfate or ethanol precipitation, acid extraction, anion or cation exchange chromatography, phosphocellulose chromatography, hydrophobic interaction chromatography, hydroxylapatite chromatography and lectin chromatography.

The hyperimmune serum reactive antigens and fragments thereof according to the present invention can be produced by chemical synthesis as well as by biotechnological means. The latter comprise the transfection or transformation of a host cell with a vector containing a nucleic acid according to the present invention and the cultivation of the transfected or transformed host cell under conditions, which are known to the ones skilled in the art. The production method may also comprise a purification step in order to purify or isolate the polypeptide to be manufactured. In a preferred embodiment the vector is a vector according to the present invention.

The hyperimmune serum reactive antigens and fragments thereof according to the present invention may be used for the detection of the organism or organisms in a sample containing these organisms or polypeptides derived thereof. Preferably such detection is for diagnosis, more preferable for the diagnosis of a disease, most preferably for the diagnosis of a diseases related or linked to the presence or abundance of Gram-negative bacteria, especially bacteria selected from the group comprising Escherichia, Shigella and Campylobacter. More preferably, the microorganisms are selected from the group comprising Escherichia coli, Shigella flexneri, Shigella sonnei, Shigella dysenteriae, Campylobacter coli and Campylobacter jejuni, especially the microorganism is enteroaggregative E. coli, enterotoxigenic E. coli, S. flexneri or C. jejuni.

The present invention also relates to diagnostic assays such as quantitative and diagnostic assays for detecting levels of the hyperimmune serum reactive antigens and fragments thereof of the present invention in cells and tissues, including determination of normal and abnormal levels. Thus, for instance, a diagnostic assay in accordance with the invention for detecting over-expression of the polypeptide compared to normal control tissue samples may be used to detect the presence of an infection, for example, and to identify the infecting organism. Assay techniques that can be used to determine levels of a polypeptide, in a sample derived from a host are well known to those of skill in the art. Such assay methods include radioimmunoassays, competitive-binding assays, Western Blot analysis and ELISA assays. Among these, ELISAs frequently are preferred. An ELISA assay initially comprises preparing an antibody specific to the polypeptide, preferably a monoclonal antibody. In addition, a reporter antibody generally is prepared which binds to the monoclonal antibody. The reporter antibody is attached to a detectable reagent such as radioactive, fluorescent or enzymatic reagent, such as horseradish peroxidase enzyme.

The hyperimmune serum reactive antigens and fragments thereof according to the present invention may also be used for the purpose of or in connection with an array. More particularly, at least one of the hyperimmune serum reactive antigens and fragments thereof according to the present invention may be immobilized on a support. Said support typically comprises a variety of hyperimmune serum reactive antigens and fragments thereof whereby the variety may be created by using one or several of the hyperimmune serum reactive antigens and fragments thereof according to the present invention and/or hyperimmune serum reactive antigens and fragments thereof being different. The characterizing feature of such array as well as of any array in general is the fact that at a distinct or predefined region or position on said support or a surface thereof, a distinct polypeptide is immobilized. Because of this any activity at a distinct position or region of an array can be correlated with a specific polypeptide. The number of different hyperimmune serum reactive antigens and fragments thereof immobilized on a support may range from as little as 10 to several 1,000 different hyperimmune serum reactive antigens and fragments thereof. The density of hyperimmune serum reactive antigens and fragments thereof per cm² is in a preferred embodiment as little as 10 peptides/polypeptides per cm² to at least 400 different peptides/polypeptides per cm² and more particularly at least 1,000 different hyperimmune serum reactive antigens and fragments thereof per cm².

The manufacture of such arrays is known to the one skilled in the art and, for example, described in U.S. Pat. No. 5,744,309. The array preferably comprises a planar, porous or non-porous solid support having at least a first surface. The hyperimmune serum reactive antigens and fragments thereof as disclosed herein, are immobilized on said surface. Preferred support materials are, among others, glass or cellulose. It is also within the present invention that the array is used for any of the diagnostic applications described herein. Apart from the hyperimmune serum reactive antigens and fragments thereof according to the present invention also the nucleic acid molecules according to the present invention may be used for the generation of an array as described above. This applies as well to an array made of antibodies, preferably monoclonal antibodies as, among others, described herein.

In a further aspect the present invention relates to an antibody directed to any of the hyperimmune serum reactive antigens and fragments thereof, derivatives or fragments thereof according to the present invention. The present invention includes, for example, monoclonal and polyclonal antibodies, chimeric, single chain, and humanized antibodies, as well as Fab fragments, or the product of a Fab expression library. It is within the present invention that the antibody may be chimeric, i.e. that different parts thereof stem from different species or at least the respective sequences are taken from different species.

Antibodies generated against the hyperimmune serum reactive antigens and fragments thereof corresponding to a sequence of the present invention can be obtained by direct injection of the hyperimmune serum reactive antigens and fragments thereof into an animal or by administering the hyperimmune serum reactive antigens and fragments thereof to an animal, preferably a non-human. The antibody so obtained will then bind the hyperimmune serum reactive antigens and fragments thereof itself. In this manner, even a sequence encoding only a fragment of a hyperimmune serum reactive antigen and fragments thereof can be used to generate antibodies binding the whole native hyperimmune serum reactive antigen and fragments thereof. Such antibodies can then be used to isolate the hyperimmune serum reactive antigens and fragments thereof from tissue expressing those hyperimmune serum reactive antigens and fragments thereof.

For preparation of monoclonal antibodies, any technique known in the art, which provides antibodies produced by continuous cell line cultures can be used (as described originally in {Kohler, G. et al., 1975}.

Techniques described for the production of single chain antibodies (U.S. Pat. No. 4,946,778) can be adapted to produce single chain antibodies to immunogenic hyperimmune serum reactive antigens and fragments thereof according to this invention. Also, transgenic mice, or other organisms such as other mammals, may be used to express humanized antibodies to immunogenic hyperimmune serum reactive antigens and fragments thereof according to this invention.

Alternatively, phage display technology or ribosomal display could be utilized to select antibody genes with binding activities towards the hyperimmune serum reactive antigens and fragments thereof either from repertoires of PCR amplified v-genes of lymphocytes from humans screened for possessing respective target antigens or from naive libraries {McCafferty, I. et al., 1990}; {Marks, J. et al., 1992}. The affinity of these antibodies can also be improved by chain shuffling {Clackson, T. et al, 1991}.

If two antigen binding domains are present, each domain may be directed against a different epitope—termed ‘bispecific’ antibodies.

The above-described antibodies may be employed to isolate or to identify clones expressing the hyperimmune serum reactive antigens and fragments thereof or purify the hyperimmune serum reactive antigens and fragments thereof of the present invention by attachment of the antibody to a solid support for isolation and/or purification by affinity chromatography.

Thus, among others, antibodies against the hyperimmune serum reactive antigens and fragments thereof of the present invention may be employed to inhibit and/or treat infections, particularly bacterial infections and especially infections arising from enteroaggregative E. coli, enterotoxigenic E. coli, S. flexneri and/or C. jejuni.

Hyperimmune serum reactive antigens and fragments thereof include antigenically, epitopically or immunologically equivalent derivatives, which form a particular aspect of this invention. The term “antigenically equivalent derivative” as used herein encompasses a hyperimmune serum reactive antigen and fragments thereof or its equivalent which will be specifically recognized by certain antibodies which, when raised to the protein or hyperimmune serum reactive antigen and fragments thereof according to the present invention, interfere with the interaction between pathogen and mammalian host. The term “immunologically equivalent derivative” as used herein encompasses a peptide or its equivalent which when used in a suitable formulation to raise antibodies in a vertebrate, the antibodies act to interfere with the interaction between pathogen and mammalian host

The hyperimmune serum reactive antigens and fragments thereof, such as an antigenically or immunologically equivalent derivative or a fusion protein thereof can be used as an antigen to immunize a mouse or other animal such as a rat or chicken. The fusion protein may provide stability to the hyperimmune serum reactive antigens and fragments thereof. The antigen may be associated, for example by conjugation, with an immunogenic carrier protein, for example bovine serum albumin (BSA) or keyhole limpet haemocyanin (KLH). Alternatively, an antigenic peptide comprising multiple copies of the protein or hyperimmune serum reactive antigen and fragments thereof, or an antigenically or immunologically equivalent hyperimmune serum reactive antigen and fragments thereof, may be sufficiently antigenic to improve immunogenicity so as to obviate the use of a carrier.

Preferably the antibody or derivative thereof is modified to make it less immunogenic in the individual. For example, if the individual is human the antibody may most preferably be “humanized”, wherein the complimentarity determining region(s) of the hybridoma-derived antibody has been transplanted into a human monoclonal antibody, for example as described in {Jones, P. et al., 1986} or {Tempest, P. et al., 1991}.

The use of a polynucleotide of the invention in genetic immunization will preferably employ a suitable delivery method such as direct injection of plasmid DNA into muscle, delivery of DNA complexed with specific protein carriers, coprecipitation of DNA with calcium phosphate, encapsulation of DNA in various forms of liposomes, particle bombardment {Tang, D. et al., 1992}, {Eisenbraun, M. et al., 1993} and in vivo infection using cloned retroviral vectors {Seeger, C. et al., 1984}.

In a further aspect the present invention relates to a peptide binding to any of the hyperimmune serum reactive antigens and fragments thereof according to the present invention, and a method for the manufacture of such peptides whereby the method is characterized by the use of the hyperimmune serum reactive antigens and fragments thereof according to the present invention and the basic steps are known to the one skilled in the art.

Such peptides may be generated by using methods according to the state of the art such as phage display or ribosome display. In case of phage display, basically a library of peptides is generated, in form of phages, and this kind of library is contacted with the target molecule, in the present case a hyperimmune serum reactive antigen and fragments thereof according to the present invention. Those peptides binding to the target molecule are subsequently removed, preferably as a complex with the target molecule, from the respective reaction It is known to the one skilled in the art that the binding characteristics, at least to a certain extent, depend on the particularly realized experimental set-up such as the salt concentration and the like. After separating those peptides binding to the target molecule with a higher affinity or a bigger force, from the non-binding members of the library, and optionally also after removal of the target molecule from the complex of target molecule and peptide, the respective peptide(s) may subsequently be characterised. Prior to the characterisation optionally an amplification step is realized such as, e.g. by propagating the peptide encoding phages. The characterisation preferably comprises the sequencing of the target binding peptides. Basically, the peptides are not limited in their lengths, however preferably peptides having a length from about 8 to 20 amino acids are preferably obtained in the respective methods. The size of the libraries may be about 10² to 10¹⁸, preferably 10⁸ to 10¹⁵ different peptides, however, is not limited thereto.

A particular form of target binding hyperimmune serum reactive antigens and fragments thereof are the so-called “anticalines” which are, among others, described in German patent application DE 197 42 706.

In a further aspect the present invention relates to functional nucleic acids interacting with any of the hyperimmune serum reactive antigens and fragments thereof according to the present invention, and a method for the manufacture of such functional nucleic acids whereby the method is characterized by the use of the hyperimmune serum reactive antigens and fragments thereof according to the present invention and the basic steps are known to the one skilled in the art. The functional nucleic acids are preferably aptamers and spiegelmers.

Aptamers are D-nucleic acids, which are either single stranded or double stranded and which specifically interact with a target molecule. The manufacture or selection of aptamers is, e.g. described in European patent EP 0 533 838. Basically the following steps are realized. First, a mixture of nucleic acids, i.e. potential aptamers, is provided whereby each nucleic acid typically comprises a segment of several, preferably at least eight subsequent randomised nucleotides. This mixture is subsequently contacted with the target molecule whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture having an increased ratio of nucleic acids specifically binding to the target from which the final binding nucleic add is then optionally selected. These specifically binding nucleic acid(s) are referred to as aptamers. It is obvious that at any stage of the method for the generation or identification of the aptamers samples of the mixture of individual nucleic acids may be taken to determine the sequence thereof using standard techniques. It is within the present invention that the aptamers may be stabilized such as, e.g., by introducing defined chemical groups which are known to the one skilled in the art of generating aptamers. Such modification may for example reside in the introduction of an amino group at the 2′-position of the sugar moiety of the nucleotides. Aptamers are currently used as therapeutical agents. However, it is also within the present invention that the thus selected or generated aptamers may be used for target validation and/or as lead substance for the development of medicaments, preferably of medicaments based on small molecules. This is actually done by a competition assay whereby the specific interaction between the target molecule and the aptamer is inhibited by a candidate drug whereby upon replacement of the aptamer from the complex of target and aptamer it may be assumed that the respective drug candidate allows a specific inhibition of the interaction between target and aptamer, and if the interaction is specific, said candidate drug will, at least in principle, be suitable to block the target and thus decrease its biological availability or activity in a respective system comprising such target. The thus obtained small molecule may then be subject to further derivatisation and modification to optimise its physical, chemical, biological and/or medical characteristics such as toxicity, specificity, biodegradability and bioavailability.

Spiegelmers and their generation or manufacture is based on a similar principle. The manufacture of spiegelmers is described in international patent application WO 98/08856. Spiegelmers are L-nucleic acids, which means that they are composed of L-nucleotides rather than D-nucleotides as aptamers are. Spiegelmers are characterized by the fact that they have a very high stability in biological systems and, comparable to aptamers, specifically interact with the target molecule against which they are directed. In the process of generating spiegelmers, a heterogeonous population of D-nucleic acids is created and this population is contacted with the optical antipode of the target molecule, in the present case for example with the D-enantiomer of the naturally occurring L-enantiomer of the hyperimmune serum reactive antigens and fragments thereof according to the present invention. Subsequently, those D-nucleic adds are separated which do not interact with the optical antipode of the target molecule. But those D-nucleic adds interacting with the optical antipode of the target molecule are separated, optionally identified and/or sequenced and subsequently the corresponding L-nucleic acids are synthesized based on the nucleic acid sequence information obtained from the D-nucleic acids. These L-nucleic acids, which are identical in terms of sequence with the aforementioned D-nucleic adds interacting with the optical antipode of the target molecule, will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

In a further aspect the present invention relates to functional nucleic adds interacting with any of the nucleic acid molecules according to the present invention, and a method for the manufacture of such functional nucleic acids whereby the method is characterized by the use of the nucleic acid molecules and their respective sequences according to the present invention and the basic steps are known to the one skilled in the art. The functional nucleic acids are preferably ribozymes, antisense oligonucleotides and siRNA.

Ribozymes are catalytically active nucleic adds, which preferably consist of RNA, which basically comprises two moieties. The first moiety shows a catalytic activity whereas the second moiety is responsible for the specific interaction with the target nucleic acid, in the present case the nucleic acid coding for the hyperimmune serum reactive antigens and fragments thereof according to the present invention. Upon interaction between the target nucleic acid and the second moiety of the ribozyme, typically by hybridisation and Watson-Crick base pairing of essentially complementary stretches of bases on the two hybridising strands, the catalytically active moiety may become active which means that it catalyses, either intramolecularly or intermolecularly, the target nucleic acid in case the catalytic activity of the ribozyme is a phosphodiesterase activity. Subsequently, there may be a further degradation of the target nucleic acid, which in the end results in the degradation of the target nucleic acid as well as the protein derived from the said target nucleic acid. Ribozymes, their use and design principles are known to the one skilled in the art, and, for example described in {Doherty, E. et al. 2001} and {Lewin, A. et al., 2001}.

The activity and design of antisense oligonucleotides for the manufacture of a medicament and as a diagnostic agent, respectively, is based on a similar mode of action. Basically, antisense oligonucleotides hybridise based on base complementarity, with a target RNA, preferably with a mRNA, thereby activating RNase H. RNase H is activated by both phosphodiester and phosphorothioate-coupled DNA. Phosphodiester-coupled DNA, however, is rapidly degraded by cellular nucleases with the exception of phosphorothioate-coupled DNA. These resistant, non-naturally occurring DNA derivatives do not inhibit RNase H upon hybridisation with RNA. In other words, antisense polynudeotides are only effective as DNA RNA hybride complexes. Examples for this kind of antisense oligonucleotides are described, among others, in U.S. Pat. No. 5,849,902 and U.S. Pat. No. 5,989,912. In other words, based on the nucleic acid sequence of the target molecule which in the present case are the nucleic acid molecules for the hyperimmune serum reactive antigens and fragments thereof according to the present invention, either from the target protein from which a respective nucleic acid sequence may in principle be deduced, or by knowing the nucleic acid sequence as such, particularly the mRNA, suitable antisense oligonucleotides may be designed base on the principle of base complementarity.

Particularly preferred are antisense-oligonucleotides, which have a short stretch of phosphorothioate DNA (3 to 9 bases). A minimum of 3 DNA bases is required for activation of bacterial RNase H and a minimum of 5 bases is required for mammalian RNase H activation. In these chimeric oligonudeotides there is a central region that forms a substrate for RNase H that is flanked by hybridising “arms” comprised of modified nucleotides that do not form substrates for RNase H. The hybridising arms of the chimeric oligonucleotides may be modified such as by 2′-O-methyl or 2′-fluoro. Alternative approaches used methylphosphonate or phosphoramidate linkages in said arms. Further embodiments of the antisense oligonudeotide useful in the practice of the present invention are P-methoxyoligonucleotides, partial P-methoxyoligodeoxyribonudeotides or P-methoxyoligonudeotides.

Of particular relevance and usefulness for the present invention are those antisense oligonudeotides as more particularly described in the above two mentioned US patents. These oligonudeotides contain no naturally occurring 5′→3′-linked nucleotides. Rather the oligonudeotides have two types of nucleotides: 2′-deoxyphosphorothioate, which activate RNase H, and 2′-modified nucleotides, which do not. The linkages between the 2′-modified nucleotides can be phosphodiesters, phosphorothioate or P-ethoxyphosphodiester. Activation of RNase H is accomplished by a contiguous RNase H-activating region, which contains between 3 and 5 2′-deoxyphosphorothioate nucleotides to activate bacterial RNase H and between 5 and 10 2′- deoxyphosphorothioate nucleotides to activate eucaryotic and, particularly, mammalian RNase H. Protection from degradation is accomplished by making the 5′ and 3′ terminal bases highly nuclease resistant and, optionally, by placing a 3′ terminal blocking group.

More particularly, the antisense oligonucleotide comprises a 5′ terminus and a 3′ terminus; and from position 11 to 59 5′→3′-linked nucleotides independently selected from the group consisting of 2′-modified phosphodiester nucleotides and 2′-modified P-alkyloxyphosphotriester nucleotides; and wherein the 5′-terminal nucleoside is attached to an RNase H-activating region of between three and ten contiguous phosphorothioate4inked deoxyribonucleotides, and wherein the 3′-terminus of said oligonucleotide is selected from the group consisting of an inverted deoxyribonucleotide, a contiguous stretch of one to three phosphorothioate 2′-modified ribonucleotides, a biotin group and a P-alkyloxyphosphotriester nucleotide.

Also an antisense oligonudeotide may be used wherein not the 5′ terminal nucleoside is attached to an RNase H-activating region but the 3′ terminal nucleoside as specified above. Also, the 5′ terminus is selected from the particular group rather than the 3′ terminus of said oligonudeotide.

The nucleic acids as well as the hyperimmune serum reactive antigens and fragments thereof according to the present invention may be used as or for the manufacture of pharmaceutical compositions, especially vaccines. Preferably such pharmaceutical composition, preferably vaccine is for the prevention or treatment of diseases caused by, related to or associated with enteroaggregative E. coli, enterotoxigenic E. coli, S. flexneri and/or C. jejuni. In so far another aspect of the invention relates to a method for inducing an immunological response in an individual, particularly a mammal, which comprises inoculating the individual with the hyperimmune serum reactive antigens and fragments thereof of the invention, or a fragment or variant thereof, adequate to produce antibodies to protect said individual from infection, particularly an infection causing diarrhea among other symptoms and most particularly enteroaggregative E. coli, enterotoxigenic E. coli, S. flexneri and/or C. jejuni infections.

Yet another aspect of the invention relates to a method of inducing an immunological response in an individual which comprises, through gene therapy or otherwise, delivering a nucleic acid functionally encoding hyperimmune serum reactive antigens and fragments thereof, or a fragment or a variant thereof, for expressing the hyperimmune serum reactive antigens and fragments thereof, or a fragment or a variant thereof in vivo in order to induce an immunological response to produce antibodies or a cell mediated T cell response, either cytokine-producing T cells or cytotoxic T cells, to protect said individual from disease, whether that disease is already established within the individual or not. One-way of administering the gene is by accelerating it into the desired cells as a coating on particles or otherwise.

A further aspect of the invention relates to an immunological composition which, when introduced into a host capable of having induced within it an immunological response, induces an immunological response in such host, wherein the composition comprises recombinant DNA which codes for and expresses an antigen of the hyperimmune serum reactive antigens and fragments thereof of the present invention. The immunological response may be used therapeutically or prophylactically and may take the form of antibody immunity or cellular immunity such as that arising from CTh or CD4+ T cells.

The hyperimmune serum reactive antigens and fragments thereof of the invention or a fragment thereof may be fused with a co-protein which may not by-itself produce antibodies, but is capable of stabilizing the first protein and producing a fused protein which will have immunogenic and protective properties. This fused recombinant protein preferably further comprises an antigenic co-protein, such as Glutathione-S-transferase (GST) or beta-galactosidase, relatively large co-proteins which solubilise the protein and facilitate production and purification thereof. Moreover, the co-protein may act as an adjuvant in the sense of providing a generalized stimulation of the immune system. The co-protein may be attached to either the amino or carboxy terminus of the first protein.

Also, provided by this invention are methods using the described nucleic acid molecule or particular fragments thereof in such genetic immunization experiments in animal models of infection with enteroaggregative E. coli, enterotoxigenic E. coli, S. flexneri and/or C. jejuni. Such fragments will be particularly useful for identifying protein epitopes able to provoke a prophylactic or therapeutic immune response. This approach can allow for the subsequent preparation of monoclonal antibodies of particular value from the requisite organ of the animal successfully resisting or clearing infection for the development of prophylactic agents or therapeutic treatments of enteroaggregative E. coli, enterotoxigenic E. coli, S. flexneri and/or C. jejuni infection in mammals, particularly humans.

The hyperimmune serum reactive antigens and fragments thereof may be used as an antigen for vaccination of a host to produce specific antibodies which protect against invasion of bacteria, for example by blocking adherence of bacteria to damaged tissue. Examples of tissue damage include wounds in skin or connective tissue and mucosal tissues caused e.g. by viral infection (esp. respiratory, such as the flu) mechanical, chemical or thermal damage or by implantation of indwelling devices, or wounds in the mucous membranes, such as the mouth, mammary glands, urethra or vagina.

The present invention also includes a vaccine formulation, which comprises the immunogenic recombinant protein together with a suitable carrier. Since the protein may be broken down in the stomach, it is preferably administered parenterally, including, for example, administration that is subcutaneous, intramuscular, intravenous, intradermal intranasal or transdermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampoules and vials, and may be stored in a freeze-dried condition requiring only the addition of the sterile liquid carrier immediately prior to use. The vaccine formulation may also include adjuvant systems for enhancing the immunogenicity of the formulation, such as oil-in-water systems and other systems known in the art. The dosage will depend on the specific activity of the vaccine and can be readily determined by routine experimentation.

According to another aspect, the present invention relates to a pharmaceutical composition comprising such a hyperimmune serum-reactive antigen or a fragment thereof as provided in the present invention for enteroaggregative E. coli, enterotoxigenic E. coli, S. flexneri and/or C. jejune. Such a pharmaceutical composition may comprise one preferably at least two or more hyperimmune serum reactive antigens or fragments thereof against enteroaggregative E. coli, enterotoxigenic E. coli, S. flexneri and/or C. jejuni. Optionally, such enteroaggregative E. coli, enterotoxigenic E. coli, S. flexneri and/or C. jejuni hyperimmune serum reactive antigens or fragments thereof may also be combined with antigens against even further pathogens in a combination pharmaceutical composition. Preferably, said pharmaceutical composition is a vaccine for preventing or treating an infection caused by enteroaggregative E coli, enterotoxigenic E. coli, S. flexneri and/or C. jejuni and/or other pathogens against which the antigens have been included in the vaccine.

According to a further aspect, the present invention relates to a pharmaceutical composition comprising a nucleic acid molecule encoding a hyperimmune serum-reactive antigen or a fragment thereof as identified above for enteroaggregative E. coli, enterotoxigenic E. coli, S. flexneri and/or C. jejuni. Such a pharmaceutical composition may comprise one or more nucleic acid molecules encoding hyperimmune serum reactive antigens or fragments thereof against enteroaggregative E. coli, enterotoxigenic E. coli, S. flexneri and/or C. jejuni. Optionally, such enteroaggregative E. coli, enterotoxigenic E. coli, S. flexneri and/or C. jejuni nucleic acid molecules encoding hyperimmune serum reactive antigens or fragments thereof may also be combined with nucleic acid molecules encoding antigens against other pathogens in a combination pharmaceutical composition. Preferably, said pharmaceutical composition is a vaccine for preventing or treating an infection caused by enteroaggregative E. coli, enterotoxigenic E. coli, S. flexneri and/or C. jejuni and/or other pathogens against which the antigens have been included in the vaccine.

The pharmaceutical composition may contain any suitable auxiliary substances, such as buffer substances, stabilisers or further active ingredients, especially ingredients known in connection of pharmaceutical composition and/or vaccine production.

A preferable carrier/or excipient for the hyperimmune serum-reactive antigens, fragments thereof or a coding nucleic acid molecule thereof according to the present invention is an immunostimulatory compound for further stimulating the immune response to the given hyperimmune serum-reactive antigen, fragment thereof or a coding nucleic acid molecule thereof. Preferably the immunostimulatory compound in the pharmaceutical preparation according to the present invention is selected from the group of polycationic substances, especially polycationic peptides, immunostimulatory nucleic acids molecules, preferably immunostimulatory deoxynucleotides, alum, Freund's complete adjuvants, Freund's incomplete adjuvants, neuroactive compounds, especially human growth hormone, or combinations thereof.

It is also within the scope of the present invention that the pharmaceutical composition, especially vaccine, comprises apart from the hyperimmune serum reactive antigens, fragments thereof and/or coding nucleic acid molecules thereof according to the present invention other compounds which are biologically or pharmaceutically active. Preferably, the vaccine composition comprises at least one polycationic peptide. The polycationic compound(s) to be used according to the present invention may be any polycationic compound, which shows the characteristic effects according to the WO 97/30721. Preferred polycationic compounds are selected from basic polypeptides, organic polycations, basic polyamino acids or mixtures thereof. These polyamino acids should have a chain length of at least 4 amino acid residues (WO 97/30721). Especially preferred are substances like polylysine, polyarginine and polypeptides containing more than 20%, especially more than 50% of basic amino acids in a range of more than 8, especially more than 20, amino acid residues or mixtures thereof. Other preferred polycations and their pharmaceutical compositions are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be anti-microbial with properties as reviewed in {Ganz, T., 1999}. These (poly)peptides may be of prokaryotic or animal or plant origin or may be produced chemically or recombinantly (WO 02/13857). Peptides may also belong to the class of defensins (WO 02/13857). Sequences of such peptides can be, for example, found in the Antimicrobial Sequences Database under the following internet address:
http://www.bbcm.univ.trieste.it/˜tossi/pag2.html

Such host defence peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substances in the present invention are cathelicidin derived antimicrobial peptides or derivatives thereof (International patent application WO 02/13857, incorporated herein by reference), especially antimicrobial peptides derived from mammalian cathelicidin, preferably from human, bovine or mouse.

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide, which has the amino acid sequence NH₂-RLAGLLRKGGEKIGEKLKKIGOKIKNFFQKLVPQPE-COOH. Related or derived cathelin substances contain the whole or parts of the cathelin sequence with at least 15-20 amino add residues. Derivations may include the substitution or modification of the natural amino acids by amino acids, which are not among the 20 standard amino adds. Moreover, further cationic residues may be introduced into such cathelin molecules. These cathelin molecules are preferred to be combined with the antigen. These cathelin molecules surprisingly have turned out to be also effective as an adjuvant for an antigen without the addition of further adjuvants. It is therefore possible to use such cathelin molecules as efficient adjuvants in vaccine formulations with or without further immunactivating substances.

Another preferred polycationic substance to be used according to the present invention is a synthetic peptide containing at least 2 KLK-motifs separated by a linker of 3 to 7 hydrophobic amino acids (International patent application WO 02/32451, incorporated herein by reference).

The pharmaceutical composition of the present invention may further comprise immunostimulatory nucleic acid(s). Immunostimulatory nucleic adds are e.g. neutral or artificial CpG containing nucleic acids, short stretches of nucleic acids derived from non-vertebrates or in form of short oligonucleotides (ODNs) containing non-methylated cytosine-guanine di-nucleotides (CpG) in a certain base context (e.g. described in WO 96/02555). Alternatively, also nucleic acids based on inosine and cytidine as e.g. described in the WO 01/93903, or deoxynucleic acids containing deoxy-inosine and/or deoxyuridine residues (described in WO 01/93905 and PCT/EP 02/05448, incorporated herein by reference) may preferably be used as immunostimulatory nucleic acids for the present invention. Preferablly, the mixtures of different immunostimulatory nucleic acids may be used according to the present invention.

It is also within the present invention that any of the aforementioned polycationic compounds is combined with any of the immunostimulatory nucleic acids as aforementioned. Preferably, such combinations are according to the ones as described in WO 01/93905, WO 02/32451, WO 01/54720, WO 01/93903, WO 02/13857 and PCT/EP 02/05448 and the Austrian patent application A 1924/2001, incorporated herein by reference.

In addition or alternatively such vaccine composition may comprise apart from the hyperimmune serum reactive antigens and fragments thereof, and the coding nucleic add molecules thereof according to the present invention a neuroactive compound. Preferably, the neuroactive compound is human growth factor as, e.g. described in WO 01/24822. Also preferably, the neuroactive compound is combined with any of the polycationic compounds and/or immunostimulatory nucleic acids as afore-mentioned.

In a further aspect the present invention is related to a pharmaceutical composition. Such pharmaceutical composition is, for example, the vaccine described herein. Also a pharmaceutical composition is a pharmaceutical composition which comprises any of the following compounds or combinations thereof the nucleic acid molecules according to the present invention, the hyperimmune serum reactive antigens and fragments thereof according to the present invention, the vector according to the present invention, the cells according to the present invention, the antibody according to the present invention, the functional nucleic adds according to the present invention and the binding peptides such as the anticalines according to the present invention, any agonists and antagonists screened as described herein. In connection therewith any of these compounds may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a hyperimmune serum reactive antigen and fragments thereof of the invention and a pharmaceutically acceptable carrier or excipient. Such carriers may include, but are not limited to, saline, buffered saline, dextrose, water, glycerol, ethanol and combinations thereof. The formulation should suit the mode of administration.

The pharmaceutical compositions may be administered in any effective, convenient manner including, for instance, administration by topical, oral, anal, vaginal, intravenous, intraperitoneal, intramuscular, subcutaneous, intranasal, intratracheal or intradermal routes among others.

In therapy or as a prophylactic, the active agent may be administered to an individual as an injectable composition, for example as a sterile aqueous dispersion, preferably isotonic.

Alternatively the composition may be formulated for topical application, for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis.

A vaccine composition is conveniently in injectable form. Conventional adjuvants may be employed to enhance the immune response. A suitable unit dose for vaccination is 0.05-5 μg antigen/per kg of body weight, and such dose is preferably administered 1-3 times and with an interval of 1-3 weeks.

With the indicated dose range, no adverse toxicological effects should be observed with the compounds of the invention, which would preclude their administration to suitable individuals.

In a further embodiment the present invention relates to diagnostic and pharmaceutical packs and kits comprising one or more containers filled with one or more of the ingredients of the aforementioned compositions of the invention. The ingredient(s) can be present in a useful amount, dosage, formulation or combination. Associated with such container(s) can be a notice in the form prescribed by a governmental agency regulating the manufacture, use or sale of pharmaceuticals or biological products, reflecting approval by the agency of the manufacture, use or sale of the product for human administration.

In connection with the present invention any disease related use as disclosed herein such as, e.g. use of the pharmaceutical composition or vaccine, is particularly a disease or diseased condition which is caused by, linked or associated with Escherichia, Shigella and Campylobacter, more preferably, enteroaggregative E. coli, enterotoxigenic E. coli, S. flexneri and/or C. jejuni. In connection therewith it is to be noted that enteroaggregative E. coli, enterotoxigenic E. coli, S. flexneri and C. jejuni all comprise several strains including those disclosed herein. A disease related, caused or associated with the bacterial infection to be prevented and/or treated according to the present invention includes besides others diarrheal disease, shigellosis and Guillain-Barre syndrom.

In a still further embodiment the present invention is related to a screening method using any of the hyperimmune serum reactive antigens or nucleic acids according to the present invention. Screening methods as such are known to the one skilled in the art and can be designed such that an agonist or an antagonist is screened. Preferably an antagonist is screened which in the present case inhibits or prevents the binding of any hyperimmune serum reactive antigen and fragment thereof according to the present invention to an interaction partner. Such interaction partner can be a naturally occurring interaction partner or a non-naturally occurring interaction partner.

The invention also provides a method of screening compounds to identify those, which enhance (agonist) or block (antagonist) the function of hyperimmune serum reactive antigens and fragments thereof or nucleic acid molecules of the present invention, such as its interaction with a binding molecule. The method of screening may involve high-throughput.

For example, to screen for agonists or antagonists, the interaction partner of the nucleic acid molecule and nucleic acid, respectively, according to the present invention, maybe a synthetic reaction mix, a cellular compartment, such as a membrane, cell envelope or cell wall, or a preparation of any thereof, may be prepared from a cell that expresses a molecule that binds to the hyperimmune serum reactive antigens and fragments thereof of the present invention. The preparation is incubated with labelled hyperimmune serum reactive antigens and fragments thereof in the absence or the presence of a candidate molecule, which may be an agonist or antagonist. The ability of the candidate molecule to bind the binding molecule is reflected in decreased binding of the labelled ligand. Molecules which bind gratuitously, i.e., without inducing the functional effects of the hyperimmune serum reactive antigens and fragments thereof, are most likely to be good antagonists. Molecules that bind well and elicit functional effects that are the same as or closely related to the hyperimmune serum reactive antigens and fragments thereof are good agonists.

The functional effects of potential agonists and antagonists may be measured, for instance, by determining the activity of a reporter system following interaction of the candidate molecule with a cell or appropriate cell preparation, and comparing the effect with that of the hyperimmune serum reactive antigens and fragments thereof of the present invention or molecules that elicit the same effects as the hyperimmune serum reactive antigens and fragments thereof. Reporter systems that may be useful in this regard include but are not limited to colorimetric labelled substrate converted into product, a reporter gene that is responsive to changes in the functional activity of the hyperimmune serum reactive antigens and fragments thereof, and binding assays known in the art.

Another example of an assay for antagonists is a competitive assay that combines the hyperimmune serum reactive antigens and fragments thereof of the present invention and a potential antagonist with membrane-bound binding molecules, recombinant binding molecules, natural substrates or ligands, or substrate or ligand mimetics, under appropriate conditions for a competitive inhibition assay. The hyperimmune serum reactive antigens and fragments thereof can be labelled such as by radioactivity or a colorimetric compound, such that the molecule number of hyperimmune serum reactive antigens and fragments thereof bound to a binding molecule or converted to product can be determined accurately to assess the effectiveness of the potential antagonist.

Potential antagonists include small organic molecules, peptides, polypeptides and antibodies that bind to a hyperimmune serum reactive antigen and fragments thereof of the invention and thereby inhibit or extinguish its acitivity. Potential antagonists also may be small organic molecules, a peptide, a polypeptide such as a closely related protein or antibody that binds to the same sites on a binding molecule without inducing functional activity of the hyperimmune serum reactive antigens and fragments thereof of the invention.

Potential antagonists include a small molecule, which binds to and occupies the binding site of the hyperimmune serum reactive antigens and fragments thereof thereby preventing binding to cellular binding molecules, such that normal biological activity is prevented. Examples of small molecules include but are not limited to small organic molecules, peptides or peptide-like molecules.

Other potential antagonists include antisense molecules (see {Okano, H. et al., 1991}; OLIGODEOXYNUCLEOTIDES AS ANTISENSE INHIBITORS OF GENE EXPRESSION; CRC Press, Boca Raton, Fla. (1988), for a description of these molecules).

Preferred potential antagonists include derivatives of the hyperimmune serum reactive antigens and fragments thereof of the invention.

As used herein the activity of a hyperimmune serum reactive antigen and fragment thereof according to the present invention is its capability to bind to any of its interaction partner or the extent of such capability to bind to its or any interaction partner.

In a particular aspect, the invention provides the use of the hyperimmune serum reactive antigens and fragments thereof, nucleic acid molecules or inhibitors of the invention to interfere with the initial physical interaction between a pathogen and mammalian host responsible for sequelae of infection. In particular the molecules of the invention may be used: i) in the prevention of adhesion of enteroaggregative E. coli, enterotoxigenic E. coli, S. flexneri and/or C. jejuni to mammalian extracellular matrix proteins at mucosal surfaces and on in-dwelling devices or to extracellular matrix proteins in wounds; ii) to block bacterial adhesion between mammalian extracellular matrix proteins and bacterial proteins which mediate tissue damage or invasion iii) or lead to evasion of immune defense; iv) to block the normal progression of pathogenesis in infections initiated other than by the implantation of in-dwelling devices or by other surgical techniques, e.g. through inhibiting nutrient acquisition.

Each of the DNA coding sequences provided herein may be used in the discovery and development of antibacterial compounds. The encoded protein upon expression can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The antagonists and agonists may be employed, for instance, to inhibit diseases arising from infection with Escherichia, Shigella and Campylobacter, especially enteroaggregative E. coli, enterotoxigenic E. coli, S. flexneri and/or C. jejuni, such as diarrheal disease.

In a still further aspect the present invention is related to an affinity device such affinity device comprises as least a support material and any of the hyperimmune serum reactive antigens and fragments thereof according to the present invention, which is attached to the support material. Because of the specificity of the hyperimmune serum reactive antigens and fragments thereof according to the present invention for their target cells or target molecules or their interaction partners, the hyperimmune serum reactive antigens and fragments thereof allow a selective removal of their interaction partner(s) from any kind of sample applied to the support material provided that the conditions for binding are met. The sample may be a biological or medical sample, including but not limited to, fermentation broth, cell debris, cell preparation, tissue preparation, organ preparation, blood, urine, lymph liquid, liquor and the like.

The hyperimmune serum reactive antigens and fragments thereof may be attached to the matrix in a covalent or non-covalent manner. Suitable support material is known to the one skilled in the art and can be selected from the group comprising cellulose, silicon, glass, aluminium, paramagnetic beads, starch and dextrane.

The present invention is further illustrated by the following figures, examples and the sequence listing, from which further features, embodiments and advantages may be taken. It is to be understood that the present examples are given by way of illustration only and not by way of limitation of the disclosure.

In connection with the present invention

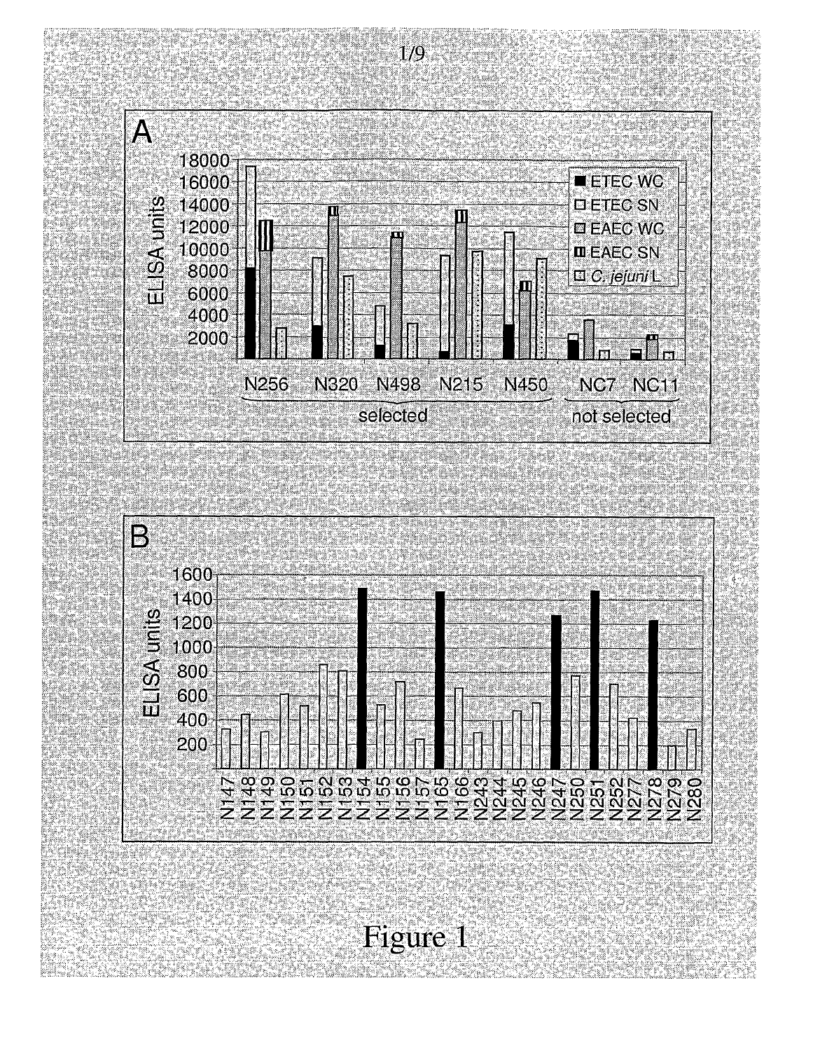

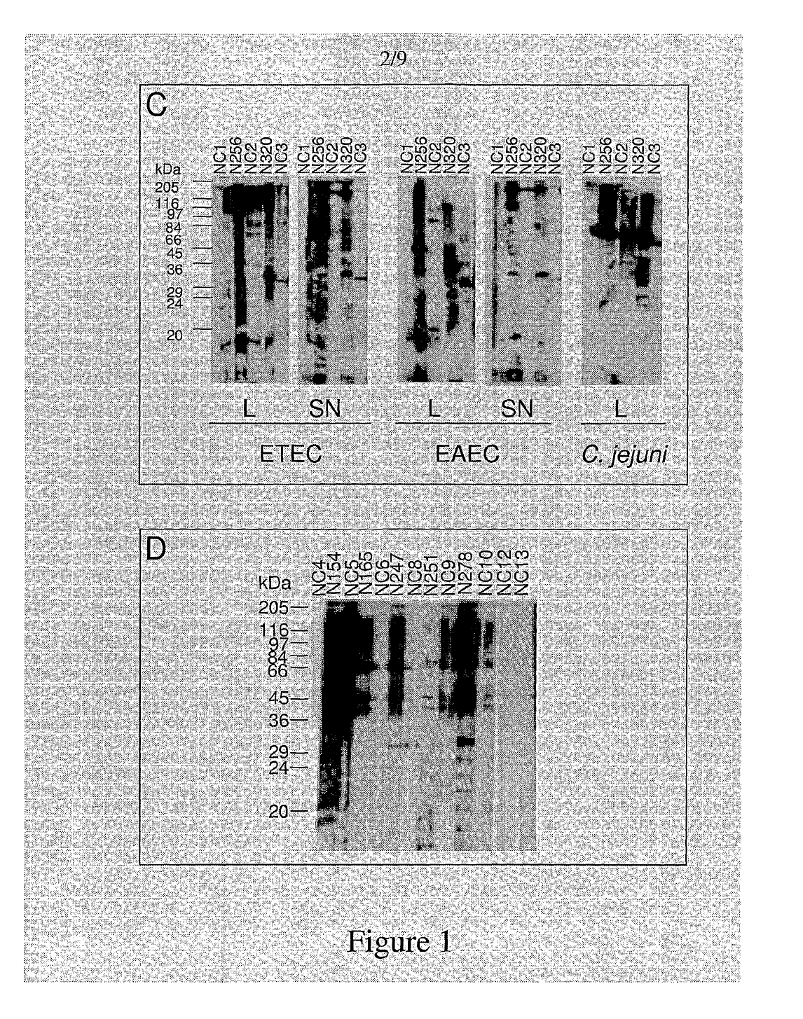

FIG. 1 shows the characterization of human sera as sources of pathogen specific antibodies.

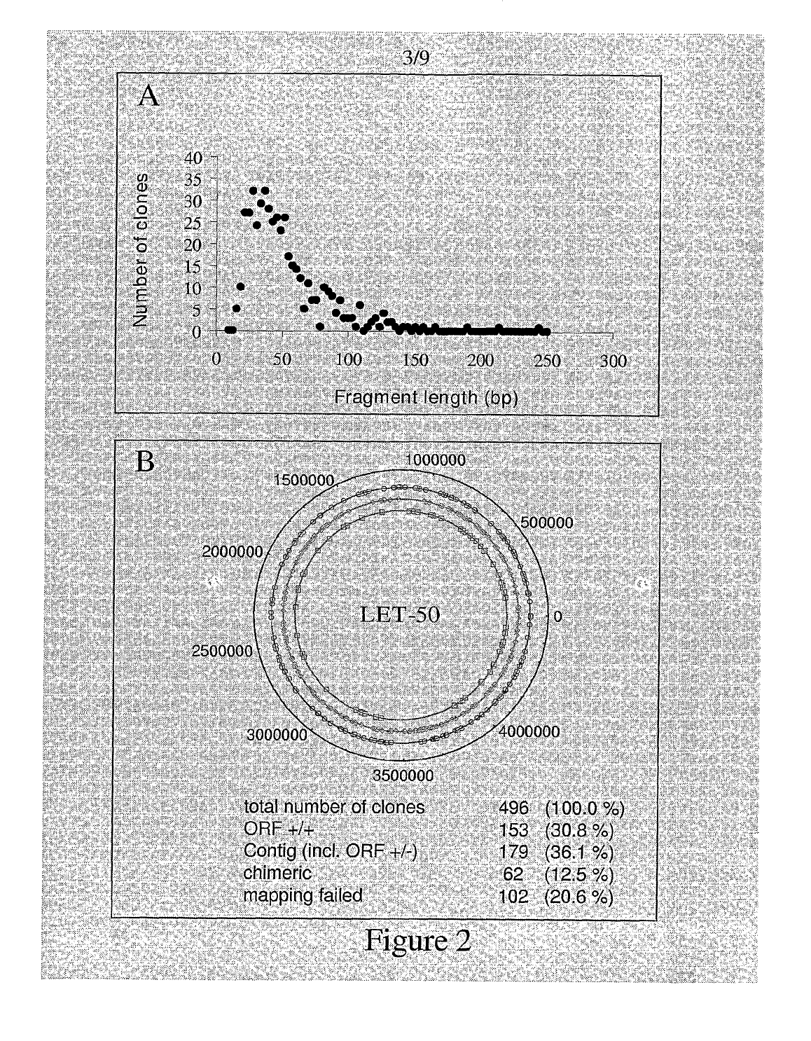

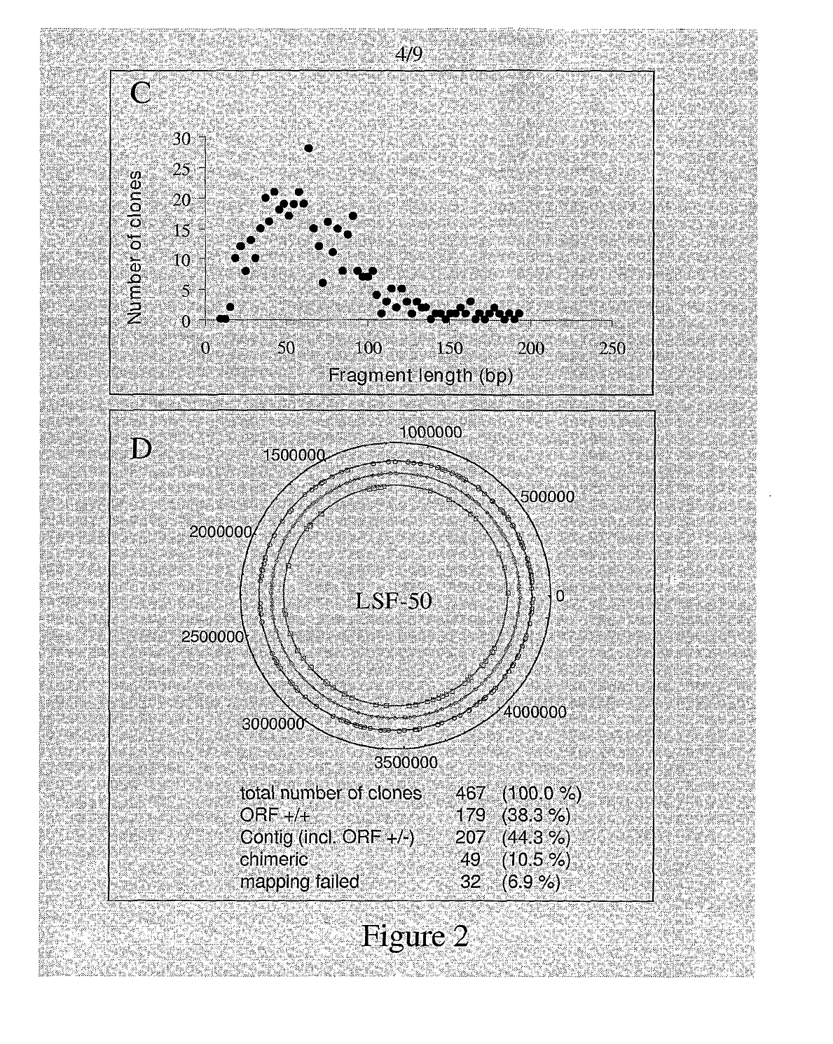

FIG. 2 shows the characterization of two small fragment genomic libraries.

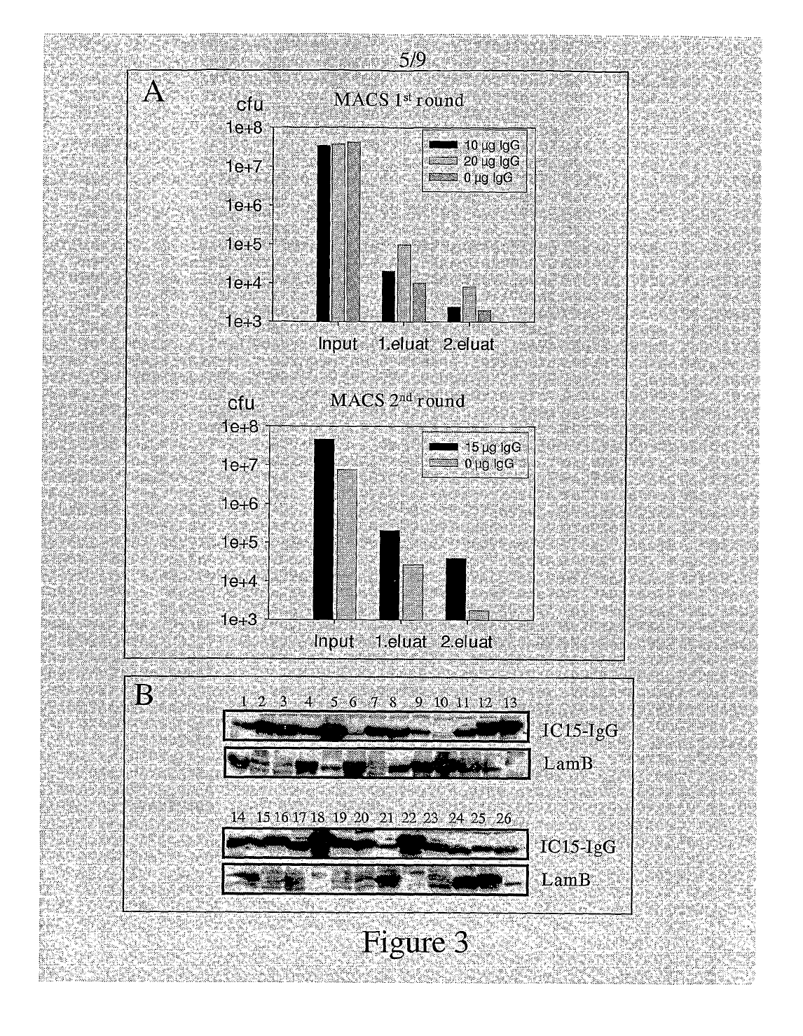

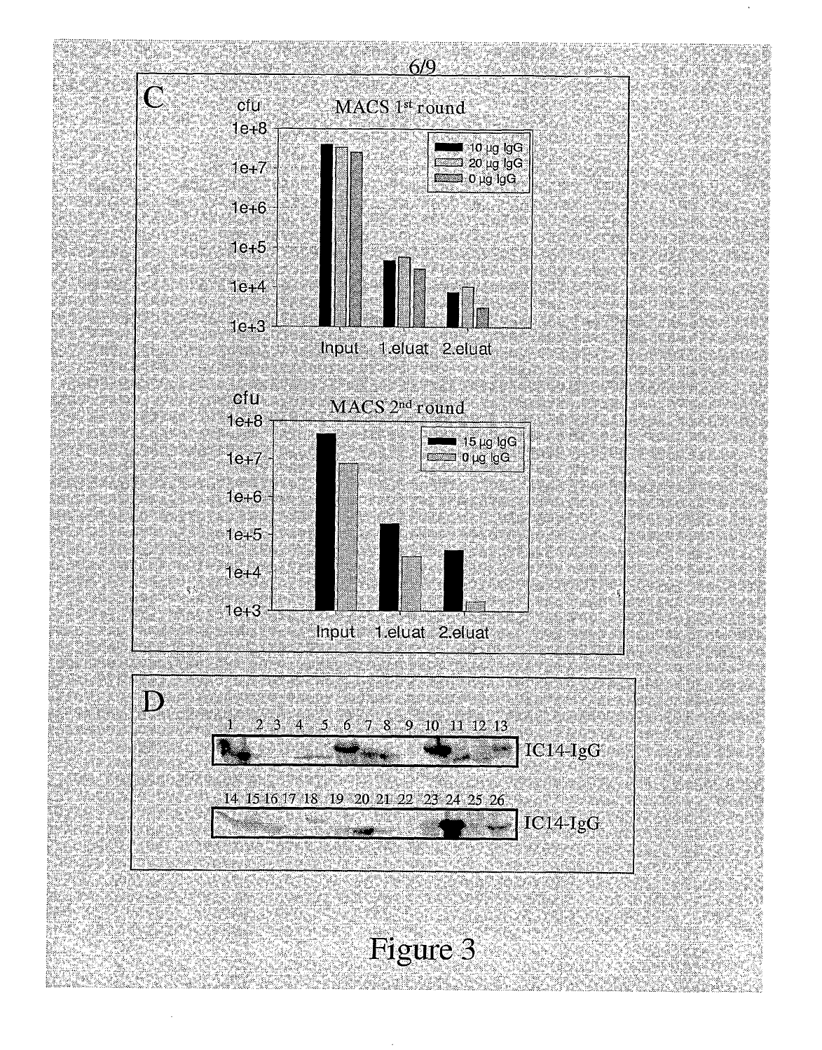

FIG. 3 shows the selection of bacterial cells by MACS using biotinylated human IgGs.

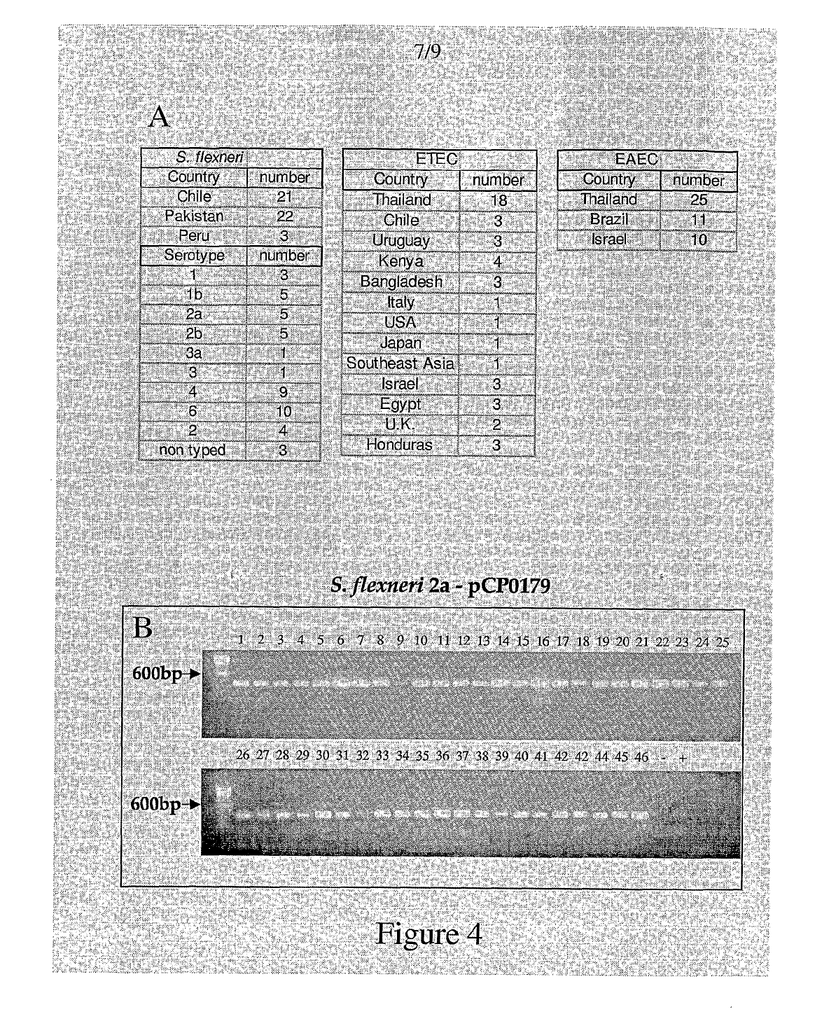

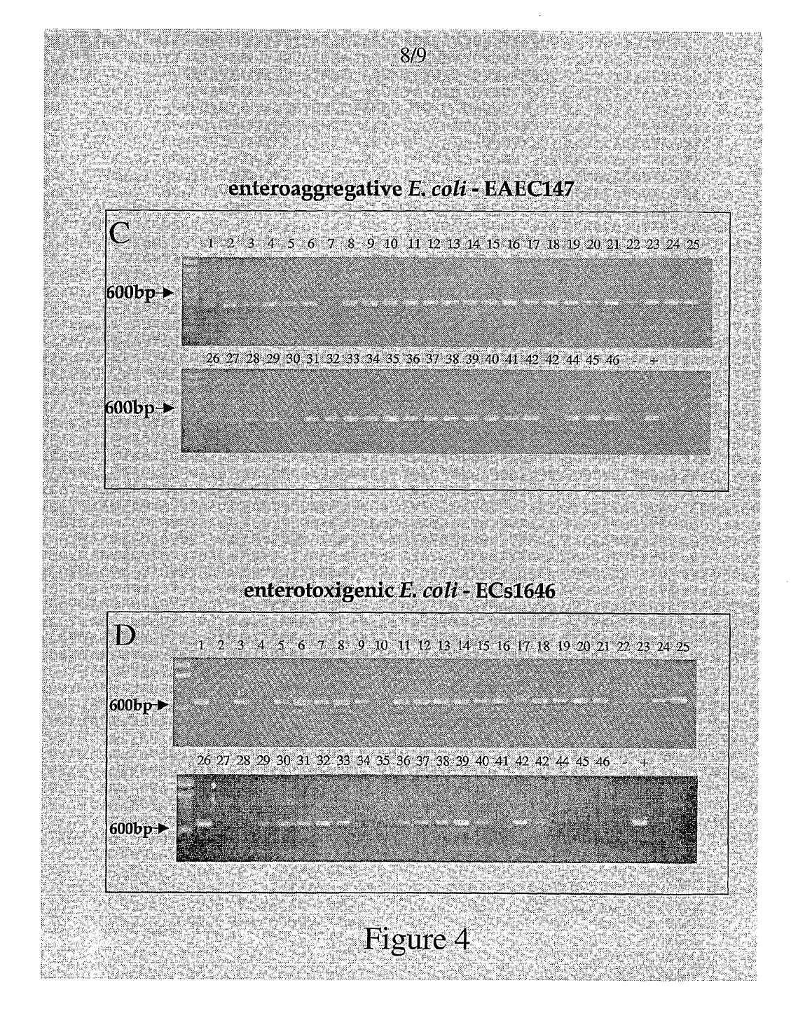

FIG. 4 shows the PCR analysis to determine the gene distribution of selected antigens in clinical isolates of the respective bacterial pathogen.

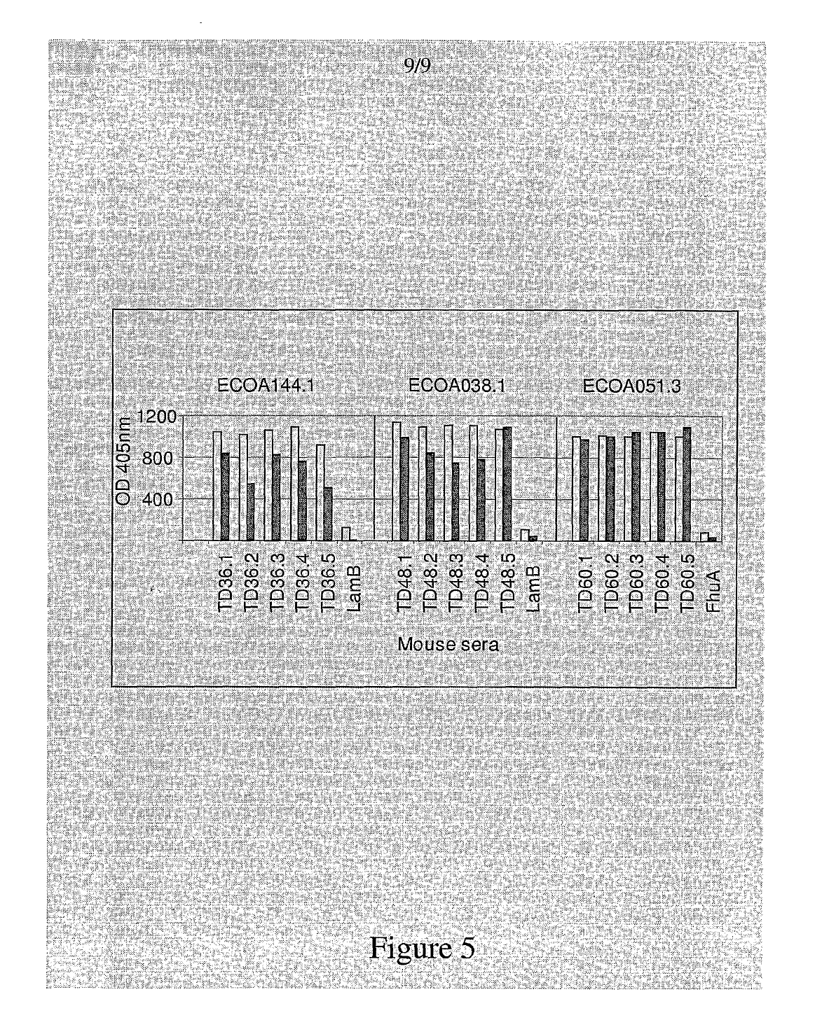

FIG. 5 shows examples for induction of epitope-specific antibodies in mice by immunization with E. coli lysates.

Table 1 shows the summary of all screens performed with genomic E. coli libraries and human serum.

Table 2 shows the summary of all screens performed with genomic S. flexneri libraries and human serum.

Table 3 shows the summary of all screens performed with genomic C. jejuni libraries and human serum.

Table 4 shows all genes identified from enteroaggregative E. coli as antigens by the genomic screens.

Table 5 shows the summary of the gene distribution analysis for a selected number of antigens in various strains of the respective bacterial species.

Table 6 shows the summary of mouse immunogenicity experiments.

Table 7 shows the summary of the peptide ELISA with human sera.

Table 8 shows the list of additional antigens identified by bacterial surface display screens.

The figures and tables to which it might be referred to in the specification are described in the following in more details.

FIG. 1 shows the characterization of human sera by measuring antibodies specific for ETEC, EAEC, C. jejuni and S. flexneri 2a by immune assays. Total IgG antibody levels were measured by standard ELISA (A) using bacterial whole cells (WC), total bacterial lysates (L) or culture supernant fractions (SN) prepared from ETEC, EAEC and C. jejuni or (B) S. flexneri whole cells. Serum samples from healthy adults living in endemic areas were analysed at two different serum dilutions. Results of representative experiments are shown for (A) with five selected and two non-selected serum samples and for (B) with 25 sera with five selected (filled bar) and 20 unselected (open bar) samples. Data are expressed as ELISA units calculated from absorbance at 405 nm at a serum dilution in the linear range of detection. (C, D) Immunoblot analysis was performed on sera pre-selected by ELISA in order to ensure multiple immune reactivity with protein antigens. Results of a representative experiment using total bacterial lysate prepared from ETEC, EAEC and C. jejuni (C) or from S. flexneri (D) reacted with human sera at 5,000× dilution are shown. IgG antibodies were detected by anti-human IgG specific secondary reagents. Not selected, low titer sera were included as negative controls (NC). Mw: molecular weight markers.

FIG. 2 (A) shows the fragment size distribution of the ETEC small fragment genomic library, LET-50. After sequencing 576 randomly selected clones, sequences were trimmed (496) to eliminate vector residues and the numbers of clones with various genomic fragment sizes were plotted. (B) shows the graphic illustration of the distribution of the same set of randomly sequenced clones of LET-50 over the E. coli K12 chromosome. (C) shows the fragment size distribution of the S. flexneri 2a small fragment genomic library, LSF-50. After sequencing 576 randomly selected clones, sequences were trimmed (467) to eliminate vector residues and the numbers of clones with various genomic fragment sizes were plotted. (D) shows the graphic illustration of the distribution of the same set of randomly sequenced clones of LSP-50 over the S. flexneri 2a chromosome. Circles indicate matching sequences to annotated ORFs in +/+ orientation and diamonds represent fully matched clones to annotated ORFs in ± orientation or to non-coding chromosomal sequences in +/+ or ± orientation. Rectangles position all clones with chimeric sequences. Numeric distances in base pairs are indicated over the circular genome for orientation. Partitioning of various clone sets within the library is given in numbers and percentage at the bottom of the figure.

FIG. 3 (A) shows the MACS selection with biotinylated human IgGs. The LET-50 library in pMAL9.1 was screened with 10-20 μg biotinylated IgG (IC15-IgG, purified from human serum). As negative control, no serum was added to the library cells for screening. Number of cells selected after the 1^{st} and 2^{nd} elution are shown for each selection round (upper and lower panel, respectively). (B) shows the reactivity of specific clones (1-26) selected by bacterial surface display as analysed by immunoblot analysis with the human serum IgG pool (IC15-IgG, 4 μl/μl) used for selection by MACS at a dilution of 1:3,000. As a loading control the same blot was also analysed with antibodies directed against the platform protein LamB at a dilution of 1:5,000. (C) shows the MACS selection with biotinylated human IC14-IgG and the LSF-50 library in pMAL9.1. (D) shows the reactivity of specific clones (1-26) selected by bacterial surface display as analysed by immunoblot analysis with the human serum IgG pool (IC14-IgG, 4 μg/μl) used for selection by MACS at a dilution of 1:3,000.

FIG. 4 (A) shows the representation of different strains of enteroaggregative E. coli, enterotoxigenic E. coli, or S. flexneri clinical isolates analysed for the gene distribution study. The geographic region is listed for all three strains, while the serotype is given in addition for S. flexneri strains. (B) to (D) shows an example for the PCR analysis for the gene distribution of one gene for each of the three analysed pathogenic species with the respective oligonucleotides and 46 clinical strains. (B) The predicted size of the PCR fragment derived from antigen pCP0179 from S. flexneri is 300 bp. (C) The predicted size of the PCR fragment derived from antigen EAEC147 from enteroaggregative E. coli is 400 bp. (D) The predicted size of the PCR fragment derived from antigen ECs1646 from enterotoxigenic E. coli is 716 bp. 1-46, strains or clinical isolates as shown under (A); −, no genomic DNA added; +, genomic DNA from the respective bacterial pathogen, which served as template for library construction.

FIG. 5 shows the measurement of epitope-specific mouse serum IgG antibody levels induced by total bacterial lysates of LamB or FhuA expressing E. coli clones with enteroaggregative or enterotoxigenic E. coli or S. flexneri 2a-derived epitopes. The figure shows a representative peptide ELISA experiment with three sets of mouse sera (5 mice in each group, 1-5) generated by epitopes expressed by bacterial clones ECOA144, ECOA038 and ECOA051, respectively. Sera were tested at two different dilutions. Open bars:, 200×; filled bars, 1000×. Biotin-labeled synthetic peptides corresponding to the respective epitopes were used in the peptide ELISA. Sera induced with E. coli lysate without pathogen-derived epitopes are indicated as PhuA or LamB.

Table 1: Immunogenic Proteins Identified from E. coli by Bacterial Surface Display.

A, 50 bp library of enteroaggregative E. coli O42 (EAEC) in LamB with IC11-IgG (819), B, 300 bp library of EAEC in fhuA with IC11-IgG (733), C, 50 bp library of EAEC in LamB with IC12-IgG (872), D, 300 bp library of EAEC in fhuA with IC12-IgG (747), E, 50 bp library of enterotoxigenic E. coli ATCC31705 (ETEC) in lamB with IC15-IgG (827), F, 300 bp library of ETEC in fhuA with IC15-IgG (503), G, 50 bp library of enteroaggregative E. coli O42 in lamB with IC16-IgG (708), H, 300 bp library of EAEC in fhuA with IC16-IgG (823), L, 50 bp library of EAEC in LamB with IC17-IgG (838), M, 300 bp library of EAEC in fhuA with IC17-IgG (783), N, 50 bp library of ETEC in lamB with IC16-IgG (777), O, 300 bp library of ETEC in fhuA with IC16-IgG (128), P, 50 bp library of ETEC in lamB with IC17-IgG (747), Q, 300 bp library of ETEC in fhuA with IC17-IgG (588); *, prediction of antigenic sequences longer than 5 amino acids was performed with the program ANTIGENIC {Kolaskar, A. et al., 1990}. Listed are the genes from E. coli O157:H7 as identified by BLAST of the determined epitope sequence against the genomic sequence of E. coli O157:H7 (http://www.tigr.org/tdb/mdb/mdbcomplete.html). In cases when the BLAST analysis could not identify a homologous sequence in E. coli O157:H7, the antigenic sequence listed was identified by BLAST against the unfinished genomic sequence of enteroaggregative E. coli O42 http://www.sanger.ac.uk/Projects/Escherichia Shigella e.g. EAEC11), or the epitope sequence was listed as such (e.g. ETLAB27).

Table 2: Immunogenic Proteins Identified from S. flexneri by Bacterial Surface Display.

A, 50 bp library of S. flexneri 2a in lamB with IC13-IgG (855), B, 300 bp library in fhuA with IC13-IgG (812), C, 50 bp library in lamB with IC14-IgG (745), D, 300 bp library in fhuA with IC14IgG (773); *, prediction of antigenic sequences longer than 5 amino acids was performed with the program ANTIGENIC {Kolaskar, A. et al. 1990}.

Table 3: Immunogenic Proteins Identified from C. jejuni by Bacterial Surface Display.

A, 50 bp library of C. jejuni NCTC 11168 in LamB with P12-IgG (628), B, 50 bp library in LamB with P12-IgG (705), C, 50 bp library in LamB with P12-IgA (691), D, 300 bp library in FhuA with P12-IgA (464), E, 50 bp library in lamB with IC11-IgG (698), F, 300 bp library in FhuA with IC11-IgG (815), *, prediction of antigenic sequences longer than 5 amino adds was performed with the program ANTIGENIC {Kolaskar, A. et al., 1990}.

Table 4: Immunogenic Proteins Identified from Enteroaggregative E. coli (EAEC) by Bacterial Surface Display.

All antigenic proteins from Table 1, for which a BLAST analysis obtained a match with the genomic sequence of E. coli O157:H7 were subjected to BLAST analysis against the unfinished and not annotated genomic sequence of enteroaggregative E. coli O42 (http://www.sanger.ac.uk/Projects/Escherichia Shigella). Listed are all sequences for which a BLAST match could be identified homologous to the enteropathogenic E. coli O157:H7 sequence. *Antigenic sequences longer than 5 amino acids were predicted with the program ANTIGENIC {Kolaskar, A. et al., 1990}.

Table 5: Gene Distribution in Enteroaggregative E. coli, Enterotoxigenic E. coli and S. flexneri Strains.

Fourty six enteroaggregative E. coli, enterotoxigenic E. coli, or S. flexneri strains as shown in FIG. 4 were tested by PCR with oligonudeotides specific for the genes encoding relevant antigens. The gene distribution lists the number of positive PCR results from all 46 tested strains and is an indication of the presence and conservation of the gene in diverse clinical isolates of the respective bacterial species. *, The source indicates the pathogenic organism from which the antigen was identified in the antigen screen. **, The column “homolog” lists homologous genes which are present in E. coli O157:H7, S. flexneri 2a or EAEC as determined by BLAST analysis. n.d., not determined.

Table 6: Immunogenicity of Antigenic Epitopes.

Enteroaggregative or enterotoxigenic E. coli or S. flexneri 2a antigens were tested for immunogenicity by immunization with E. coli clones harboring plasmids encoding the platform proteins LamB or FhuA fused to enteroaggregative or enterotoxigenic E. coli or S. flexneri 2a peptides. The presence of epitope-specific antibodies were detected and measured by peptide ELISA. Results are expressed as + to +++++, and calculated as the sum of the reactivity of individual mouse sera based on ELISA units (as indicated in FIG. 5). Location of synthetic peptides within the antigenic ORFs according to the genome annotation of the relevant strain is given in columns (aa from) and (aa to) indicating the first and last amino acid residue, respectively.

Table 7: Peptide ELISA with Peptides Derived from Enteric Pathogens.

The “Sum” represents the number of sera, for which the OD405 nm measurement was at least 0.1 OD units above the blank without coating. “From aa” and “To aa” denotes the position of the peptide relative to the full length protein as listed under the respective sequence identification number (SeqID). A, ELISA with peptides derived from E. coli and 22 human sera (N215, N256, N320, N450, N498, N159, N211, N230, N261, N353, N168, N229, N468, N502, N521, N394, N439, N165, P773, P776, P849, C24). B, ELISA with peptides derived from S. flexneri and 22 human sera (N154, N165, N247, N251, N501, N278, N423, N432, N471, N480, N211, N230, N394, N439, N468, N229, N521, N168, P773, P776, P849, C24). C, ELISA with peptides derived from C. jejuni and 22 human sera (P773, P774, P776, P817, P849, P775, P777, P850, P851, P852, P855, P856, N423, N432, N480, N211, N230, N394, N439, N468, P779, C1). D, ELISA with peptides derived from E. coli and S. flexneri and 42 human sera (N278, N423, N432, N471, N480, N159, N211, N230, N261, N353, N168, N229, N468, N502, N521, N394, N439, N542, P1277, P1303, P773, N215, N256, N320, N450, N498, P1292, P1310, P1312, P1316, N154, N165, N247, N251, N501, P773, P774, P776, P817, P849, N211, N480).

Table 8: Additional Immunogenic Proteins Identified from E. coli by Bacterial Surface Display.

A, 50 bp library of enteroaggregative E. coli O42 (EAEC) in lamB with IC11-IgG (819), B, 300 bp library of EAEC in fhuA with IC11-IgG (733), C, 50 bp library of EAEC in lamB with IC12-IgG (872), D, 300 bp library of EAEC in fhuA with IC12-IgG (747), E, 50 bp library of enterotoxigenic E. coli ATCC31705 (ETEC) in lamB with IC15-IgG (827), F, 300 bp library of ETEC in fhuA with IC15-IgG (503), G, 50 bp library of enteroaggregative E. coli O42 in lamB with IC16-IgG (708), H, 300 bp library of EAEC in fhuA with IC16-IgG (823), L, 50 bp library of EAEC in lamB with IC17-IgG (838), M 300 bp library of EAEC in fhuA with IC17-IgG (783), N, 50 bp library of ETEC in lamB with IC16-IgG (777), O, 300 bp library of ETEC in fhuA with IC16IgG (128), P, 50 bp library of ETEC in lamB with IC17-IgG (747), Q, 300 bp library of ETEC in fhuA with IC17-IgG (588). Listed are the genes from E. coli O157:H7 as identified by BLAST of the determined epitope sequence against the genomic sequence of E. coli O157:H7 (http://www.tigr.org/tdb/mdb/mdbcomplete.html). In cases when the BLAST analysis could not identify a homologous sequence in E. coli O157:H7, the antigenic sequence listed was identified by BLAST against the unfinished genomic sequence of enteroaggregative E. coli O42 (http://www.sanger.ac.uk/Projects/Escherichia Shigella, e.g. EAEC11), or the epitope sequence was listed as such (e.g. ETLAB27). *, prediction of antigenic sequences longer than 5 amino acids was performed with the program ANTIGENIC (Kolaskar and Tongaonkar, 1990).

### EXAMPLES

### Example 1

### Characterization and Selection of Human Serum Sources Based on Anti-E. coli, S. flexneri and C. jejuni Antibodies, Preparation of Antibody Screening Reagents

Experimental Procedures

Enzyme Linked Immune Assay (ELISA).

ELISA plates (Maxisorb, Millipore) were coated with 5-10 μg/ml total protein diluted in coating buffer (0.1M sodium carbonate pH 9.2). For whole cell ELISA, 10⁶ biotin-labeled and fixed bacteria were added to Streptavidin-coated ELISA plates. Two dilutions of sera (2,000×, 10,000×) were made in PBS-BSA. Highly specific Horse Radish Peroxidase (HRP)-conjugated anti-human IgG secondary antibodies (Southern Biotech) were used according to the manufacturers' recommendations (dilution: 1,000×). Antigen-antibody complexes were quantified by measuring the conversion of the substrate (ABTS) to colored product based on OD_{450 nm} readings by automatic ELISA reader (TECAN SUNRISE).

Preparation of Bacterial Antigen Extracts

Whole cells: Bacteria were grown until OD₆₀₀−0.5 and harvested by centrifugation at 4,000 rpm for 15 min at 4° C. After washing twice with PBS, bacteria was resuspended in PBS, and adjusted to a concentration of 2.5×10⁷/ml. Biotinylation was performed by incubation with biotin in PBS at room temperature for 30 min. Free biotin was removed by repeated washing and centrifugation, followed by fixation with 2% PFA at RT for 10 min.

Total bacterial lysate: Bacteria were grown overnight in LB or RPM medium (ETEC, EAEC), or Difco0001 medium (S. flexneri), or on LB agar plates and scraped off (C. jejuni) and lysed by repeated freeze-thaw cycles: incubation on dry ice/ethanol-mixture until frozen (1 min), then thawed at 37° C. (5 min): repeated 3 times. This was followed by sonication and collection of supernatant by centrifugation (4,000 rpm, 15 min, 4° C).

Culture supernatant: After removal of bacteria by centrifugation, the supernatant of overnight grown bacterial cultures was sterile filtered. Afterwards the supernatant was concentrated with AMICON ULTRA-tubes (Millipore) 10 to 100 fold depending on growth media. The protein concentration of samples was determined by Bradford assay.

Immunoblotting

Total bacterial lysate and culture supernatant samples were prepared from in vitro grown enteroaggregative or enterotoxigenic E. coli, S. flexneri 2a or C. jejuni NCTC11168. 10 to 25 μg total protein/lane was separated by SDS-PAGE using the BioRad Mini-Protean Cell electrophoresis system and proteins transferred to nitrocellulose membrane (ECL, Amersham Pharmacia). After overnight blocking in 5% milk, human sera were added at 2,000× dilution, and HRPO labeled anti-human IgG was used for detection.

Purification of Antibodies for Genomic Screening

Five sera per antibody pool were selected based on the overall anti-bacterial titers for serum used in the screening procedure. Antibodies against E. coli DH5alpha proteins were removed by incubating the heat-inactivated sera with whole cell E. coli DH5alpha cells (transformed with pHIE11, grown under the same condition as used for bacterial surface display). Highly enriched preparations of IgGs from the pooled, depleted sera were generated by protein G affinity chromatography, according to the manufacturer's instructions (UltraLink Immobilized Protein G, Pierce). IgA antibodies were purified also by affinity chromatography using biotin-labeled anti-human IgA (Southern Biotech) immobilized on Streptavidin-agarose (GIBCO BRL). The efficiency of depletion and purification was checked by ELISA measurements.

Results

The antibodies produced against enteroaggregative or enterotoxigenic E. coli, S. flexneri or C. jejuni by the human immune system and present in human sera are indicative of the in vivo expression of the antigenic proteins and their immunogenicity. These molecules are essential for the identification of individual antigens in the approach as described in the present invention, which is based on the interaction of the specific anti-bacterial antibodies and the corresponding enteroaggregative or enterotoxigenic E. coli, S. flexneri 2a or C. jejuni NCTC11168 peptides or proteins. To gain access to relevant antibody repertoires, human sera were collected from healthy adult individuals living in endemic areas (Bangladesh and Egypt). It is important to screen with antibodies from populations of at, least two different geographic regions, since enteric diseases affect individuals in various regions of the world, but prevalences of the causative pathogens vary from region to region. A distinct set of sera with 90 sampels was collected from patients with C. jejuni infections from Middle Europe, since Camplyobacter infections are common worldwide. Laboratory diagnosis of C. jejuni infections was established by Virion/Serion GmbH Campylobacter IgA ELISA.

Antibodies in serum and other body fluids induced in individuals exposed to the pathogens are crucial for antigen identification. Enteric infections are very common, and antibodies are present as a consequence of natural immunization from previous encounters, that are asymptomatic colonization, acute or chronic infections. It is likely that sera from adults living in endemic areas (multiple exposure) and having high antibody titers against enteroaggregative or enterotoxigenic E. coli, S. flexneri 2a or C. jejuni are immune (protected) from disease caused by these pathogens. Antibodies from these individuals seem to be especially valuable for the identification of the corresponding antigens.

450 endemic serum samples were collected and characterized for anti-enteroaggregative or enterotoxigenic E. coli, S. flexneri 2a or C. jejuni NCTC11168 antibodies and 90 C. jejuni-infected patient sera for C. jejuni NCTC11168 antibodies by a series of immune assays. Primary characterization was done by ELISA using different antigen preparations, such as bacterial whole cell and supernatant fractions for ETEC, EAEC and S. flexneri 2a, and total bacterial lysate for C. jejuni NCTC11168. Representative experiments are shown in FIGS. 1A and B. Antibody titers were measured and ELISA units calculated from serum dilutions in the linear range of response. Sera were ranked based on the antibody reactivity against the two complex antigenic mixtures, and the highest ones were selected for further testing by immunoblotting. This analysis confirmed a high antibody reactivity of the pre-selected sera against multiple enteroaggregative or enterotoxigenic E. coli, S. flexneri 2a or C. jejuni NCTC11168 proteins, especially when compared to not selected, low-titer sera (FIGS. 1C and D). The final selection of sera to be included in antibody-pools was based mainly on multiple immunogenic bands in immunoblotting experiments. This extensive antibody characterization approach has led to the unambiguous identification of anti-enteroaggregative or enterotoxigenic E. coli, S. flexneri 2a and C. jejuni NCTC11168 hyperimmune sera.

Selected sera were included in 8 different IgG and 1 IgA pools (5 sera in each pool) for antigen identification by bacterial surface display. IgG antibodies were purified from pooled sera by affinity chromatography and depleted of E. coli DH5alpha-reactive antibodies to avoid background in the bacterial surface display screens.

### Example 2

### Generation of Highly Random, Frame-Selected, Small-Fragment, Genomic DNA Libraries of Enteroaggregative or Enterotoxigenic E. coli, S. flexneri 2a and C. jejuni NCTC11168

Experimental Procedures

Preparation of genomic DNA. 50 ml culture medium (LB for enteroaggregative and enterotoxigenic E. coli; Difco0001 for S. flexneri 2a; modified CCDA-Preston for C. jejuni NCTC11168) were inoculated with respective bacteria from a frozen stab and grown with aeration and shaking for 18 h at 37° C. The culture was then harvested, centrifuged with 1,600× g for 15 min and the supernatant was removed. Bacterial pellets were washed 3× with PBS. Genomic DNA was prepared using the Wizard Genomic DNA Purification Kit for GRAM-negative bacteria from Promega according to the instructions of the manufacturer. After the final precipitation with ethanol, DNA was recovered by centrifuging the precipitates with 10-12,000× g, then dried on air and dissolved in ddH₂O.

Preparation of small genomic DNA fragments. Genomic DNA fragments were mechanically sheared into fragments ranging in size between 150 and 300 bp using a cup-horn sonicator (Bandelin Sonoplus UV 2200 sonicator equipped with a BB5 cup horn, 10 sec. pulses at 100% power output) or into fragments of size between 50 and 70 bp by mild DNase I treatment (Novagen). It was observed that sonication yielded a much tighter fragment size distribution when breaking the DNA into fragments of the 150-300 bp size range. However, despite extensive exposure of the DNA to ultrasonic wave-induced hydromechanical shearing force, subsequent decrease in fragment size could not be efficiently and reproducibly achieved. Therefore, fragments of 50 to 70 bp in size were obtained by mild DNase I treatment using Novagen's shotgun cleavage kit. A 1:20 dilution of DNase I provided with the kit was prepared and the digestion was performed in the presence of MnCl₂ in a 60 μl volume at 20° C. for 5 min to ensure double-stranded cleavage by the enzyme. Reactions were stopped with 2 μl of 0.5 M EDTA and the fragmentation efficiency was evaluated on a 2% TAE-agarose gel. This treatment resulted in total fragmentation of genomic DNA into near 50-70 bp fragments. Fragments were then blunt-ended twice using T4 DNA Polymerase in the presence of 100 μM each of dNTPs to ensure efficient flushing of the ends. Fragments were used immediately in ligation reactions or frozen at −20° C. for subsequent use.

Description of the vectors. The vector pMAL4.31 was constructed on a pASK-IBA backbone {Skerra, A., 1994} with the beta-lactamase (bla) gene exchanged with the Kanamycin resistance gene. In addition the bla gene was cloned into the multiple cloning site. The sequence encoding mature beta-lactamase is preceded by the leader peptide sequence of ompA to allow efficient secretion across the cytoplasmic membrane. Furthermore a sequence encoding the first 12 amino acids (spacer sequence) of mature beta-lactamase follows the ompA leader peptide sequence to avoid fusion of sequences immediately after the leader peptidase cleavage site, since e.g. clusters of positive charged amino acids in this region would decrease or abolish translocation across the cytoplasmic membrane {Kajava, A. et al., 2000}. A SmaI restriction site serves for library insertion. An upstream FseI site and a downstream NotI site, which were used for recovery of the selected fragment, flank the SmaI site. The three restriction sites are inserted after the sequence encoding the 12 amino acid spacer sequence in such a way that the bla gene is transcribed in the −1 reading frame resulting in a stop codon 15 bp after the NotI site. A +1 bp insertion restores the bla ORF so that beta-lactamase protein is produced with a consequent gain of Ampicillin resistance.

The vector pMAL9.1 was constructed by cloning the lamB gene into the multiple cloning site of pEH1 {Hashemzadeh-Bonehi, L. et al., 1998}. Subsequently, a sequence was inserted in lamB after amino acid 154, containing the restriction sites FseI, SmaI and NotI. The reading frame for this insertion was constructed in such a way that transfer of frame-selected DNA fragments excised by digestion with FseI and NotI from plasmid pMAL4.31 yields a continuous reading frame of lamB and the respective insert.

The vector pHIE11 was constructed by cloning the fluA gene into the multiple cloning site of pEH1. Thereafter, a sequence was inserted in fhuA after amino acid 405, containing the restriction site FseI, XbaI and NotI. The reading frame for this insertion was chosen in a way that transfer of frame-selected DNA fragments excised by digestion with FseI and NotI from plasmid pMAL4.31 yields a continuous reading frame of fhuA and the respective insert.

Cloning and evaluation of the library for frame selection. Genomic enteroaggregative or enterotoxigenic E. coli, S. flexneri 2a or C. jejuni NCTC11168 DNA fragments were ligated into the SmaI site of the vector pMAL4.31. Recombinant DNA was electroporated into DH10B electrocompetent E. coli cells (GIBCO BRL) and transformants plated on LB-agar supplemented with Kanamycin (50 μg/ml) and Ampicillin (50 μg/ml). Plates were incubated over night at 37° C. and colonies collected for large scale DNA extraction. A representative plate was stored and saved for collecting colonies for colony PCR analysis and large-scale sequencing. A simple colony PCR assay was used to initially determine the rough fragment size distribution as well as insertion efficiency. From sequencing data the precise fragment size was evaluated, junction intactness at the insertion site as well as the frame selection accuracy (3n+1 rule).

Cloning and evaluation of the library for bacterial surface display. Genomic DNA fragments were excised from the pMAL4.31 vector, containing the enteroaggregative or enterotoxigenic E. coli, S. flexneri 2a or C. jejuni NCTC11168 library with the restriction enzymes FseI and NotlI. The entire population of fragments was then transferred into plasmids pMAL9.1 (LamB) or pHIE11 (FhuA), which have been digested with FseI and NotI. Using these two restriction enzymes, which recognise an 8 bp GC rich sequence, the reading frame that was selected in the pMAL4.31 vector is maintained in each of the platform vectors. The plasmid library was then transformed into E. coli DH5alpha cells by electroporation. Cells were plated onto large LB-agar plates supplemented with 50 μg/ml Kanamycin and grown over night at 37° C. at a density yielding clearly visible single colonies. Cells were then scraped off the surface of these plates, washed with fresh LB medium and stored in aliquots for library screening at −80° C.

Results

Libraries for frame selection. Two libraries were generated for each bacterial pathogen in the pMAL4.31 vector with sizes of approximately 70 and 300 bp, respectively. For each library, ligation and subsequent transformation of approximately 1 μg of pMAL4.31 plasmid DNA and 50 ng of fragmented genomic enteroaggregative or enterotoxigenic E. coli, S. flexneri 2a or C. jejuni NCTC11168 DNA yielded 1.2×10⁵ to 2×10⁶ clones after frame selection. To assess the randomness of the libraries, approximately 500 to 600 randomly chosen clones of each library were sequenced. The representative bioinformatic analysis of two libraries (LET-50 and LSF-50) showed that of clones corresponding to these libraries only very few were present more than once. Furthermore, it was shown for the LET-50 library that approximately 83% of the clones fell in the size range between 25 and 100 bp with an average size of approximately 54 bp (FIG. 2A). Almost all sequences (97%) followed the 3n+1 rule, showing that the majority of clones were properly frame selected. Regarding the LSF-50 library, 98.5% of all clones possessed the proper reading-frame and 82.5% fell within the 25 to 100 bp range with an average size of 67 bp.

Bacterial surface display libraries. The display of peptides on the surface of E. coli required the transfer of the inserts from the LET-50, LEA-50, LCJ-50, LSF-50 and the LET-300, LEA-300, LCJ-300, LSF-300 libraries from the frame selection vector pMAL4.31 to the display plasmids pMAL9.1 (LamB) or pHIE11 (FhuA). Genomic DNA fragments were excised by FseI and NotI restriction and ligation of 5 ng inserts with 0.1 μg plasmtid DNA and subsequent transformation into DH5alpha cells resulted in 2.2×10⁵ to 3×10⁶ clones. The clones were scraped off the LB plates and frozen without further amplification.

### Example 3

### Identification of Highly Immunogenic Peptide Sequences from Enteroaggregative or Enterotoxigenic E. coli, S. flexneri 2a and C. jejuni NCTC11168 Using Bacterial Surface Displayed Genomic Libraries and Human Serum

Experimental Procedures

MACS screening. Approximately 2.5×10⁸ cells from a given library were grown in 5 ml LB-medium supplemented with 50 μg/ml Kanamycin for 2 h at 37° C. Expression was induced by the addition of 1 mM IPTG for 30 min. Cells were washed twice with fresh LB medium and approximately 2×10⁷ cells re-suspended in 100 μl LB medium and transferred to an Eppendorf tube.

10 to 20 μg of biotinylated, human IgGs purified from serum was added to the cells and the suspension incubated overnight at 4° C. with gentle shaking. 900 μl of LB medium was added, the suspension mixed and subsequently centrifuged for 10 min at 6,000 rpm at 4° C. (For IgA screens, 10 μg of purified IgAs were used and these captured with biotinylated anti-human-IgG secondary antibodies). Cells were washed once with 1 ml LB and then re-suspended in 100 μl LB medium. 10 μl of MACS microbeads coupled to streptavidin (Miltenyi Biotech, Germany) were added and the incubation continued for 20 min at 4° C. Thereafter 900 μl of LB medium was added and the MACS microbead cell suspension was loaded onto the equilibrated MS column (Miltenyi Biotech, Germany) which was fixed to the magnet. (The MS columns were equilibrated by washing once with 1 ml 70% EtOH and twice with 2 ml LB medium.)

The column was then washed three times with 3 ml LB medium. After removal of the magnet, cells were eluted by washing with 9 ml LB medium. After washing the column with 3 ml LB medium, the 2 ml eluate was loaded a second time on the same column and the washing and elution process repeated. The loading, washing and elution process was performed a third time, resulting in a final eluate of 2 ml.

A second and third round of screening was performed as follows. The cells from the final eluate were collected by centrifugation and re-suspended in 1 ml LB medium supplemented with 50 μg/ml Kanamycin. The culture was incubated at 37° C. for 90 min and then induced with 1 mM IPTG for 30 min. Cells were subsequently collected, washed once with 1 ml LB medium and suspended in 10 μl LB medium. 10 to 20 μg of human, biotinylated IgGs were added again and the suspension incubated over night at 4° C. with gentle shaking. All further steps were exactly the same as in the first selection round. Cells selected after two rounds of selection were plated onto LB-agar plates supplemented with 50 μg/ml Kanamycin and grown over night at 37° C.

Evaluation of selected clones by sequencing and Western blot analysis. Selected clones were grown overnight at 37° C. in 3 ml LB medium supplemented with 50 μg/ml Kanamycin to prepare plasmid DNA using standard procedures. Sequencing was performed at MWG (Germany).

For Western blot analysis approximately 10 to 20 μg of total cellular protein was separated by 10% SDS-PAGE and blotted onto HybondC membrane (Amersham Pharmacia Biotech, England). The LamB or FhuA fusion proteins were detected using human serum as the primary antibody at a dilution of approximately 1:3,000 to 1:5,000 and anti-human IgG or IgA antibodies coupled to HRP at a dilution of 1:5,000 as secondary antibodies. Detection was performed using the ECL detection kit (Amersham Pharmacia Biotech, England). Alternatively, rabbit anti-FhuA or rabbit anti-LamB polyclonal immune sera were used as primary antibodies in combination with the respective secondary antibodies coupled to HRP for the detection of the fusion proteins.

Results

Screening of bacterial surface display libraries by magnetic activated cell sorting (MACS) using biotinylated Igs. The libraries LET-50, LEA-50, LCJ-50, LSF-50 in pMAL9.1 and LET-300, LEA-300, LCJ-300, LSF-300 in pHIE11 were screened with pools of biotinylated, human IgGs and IgAs prepared from sera of healthy adults or patients (EAEC: IC11, IC12, IC16, IC17; ETEC: IC15, IC16, IC17; S. flexneri: IC13, IC14; C. jejuni: IC11, P12)) (see Example 1: Preparation of antibodies from human serum). The selection procedure was performed as described under Experimental procedures. FIG. 3A shows a representative example of a screen with the LET-50 library and IC15-IgGs. As can be seen from the colony count after the first selection cycle from MACS screening, the total number of cells recovered at the end is drastically reduced from 5×10⁷ cells to approximately 1×10⁴ cells, and the selection without antibodies showed a more pronounced reduction in cell numbers (FIG. 3A). After a second round of selection all cells from the first round were recovered with IC15-IgGs (6×10⁴), while only 2×10³ cells were recovered when no IgGs from human serum were added, dearly showing that selection was dependent on ETEC specific antibodies. To evaluate the performance of the screen, 26 selected clones were picked randomly and subjected to immunoblot analysis with the screening IC15-IgG pool (FIG. 3B). This analysis revealed that almost all selected clones showed reactivity with antibodies present in the relevant serum whereas the control strain expressing LamB without an ETEC specific insert did not react with the same serum. In general, the rate of reactivity was observed to lie within the range of 35 to 90%. Colony PCR analysis showed that all selected clones contained an insert in the expected size range.

Similar results were seen in screens with libraries from the other pathogenic organisms, S. flexneri 2a and C. jejuni. As a second example, FIGS. 3C and D show the data obtained with the small insert LSF-50 library from S. flexneri 2a in LamB and the IC14IgG antibody pool. Two rounds of MACS selection resulted in the enrichment of cells only in the presence, but not the absence of specific IgG (FIG. 3C), indicating that the selection was specific for the applied antibodies. The specific selection was then confirmed in the Western blot analysis of individual bacterial clones with the same IC14IgG antibody pool (FIG. 3D).

Subsequent sequencing of a larger number of randomly picked clones (600 to 1200) from each screen led to the identification of the gene and the corresponding peptide or protein sequence that was specifically recognized by the human serum antibodies used for screening. The frequency with which a specific clone is selected reflects at least in part the abundance and/or affinity of the specific antibodies in the serum used for selection and recognizing the epitope presented by this clone. In that regard it is striking that clones derived from some ORFs (e.g. virG, pCP0262 or ipaA from S. flexneri 2a) were picked more than 100 times, indicating their highly immunogenic property. Table 1 to 3 summarize the data obtained for all 24 performed screens for the four pathogenic bacteria. All clones that are presented in Tables 1 to 3 and Table 8 have been verified by immunoblot analysis using whole cellular extracts from single clones to show the indicated reactivity with the pool of human serum used in the respective screen. As can be seen from Tables 1 to 3 and Table 8, distinct regions of the identified ORF are identified as immunogenic, since variably sized fragments of the proteins are displayed on the surface by the platform proteins. Since the genomic sequence of enteroaggregative E. coli has been determined, but not annotated, all BLAST analyses with epitopes derived from the E. coli screens, have been first performed with the genomic sequence from enteropathogenic E. coli O157:H7. All these sequences have subsequentely also been blasted against the non-annotated preliminary sequence of enteroaggregative E. coli. Table 4 lists the genes as identified by this BLAST analysis. The identified antigenic epitopes are homologous to the ones listed in Table 1 for the E. coli O157:H7 sequences. The predicted antigenic sequences of these proteins are listed in Table 4.

It is further worth noticing that many of the genes identified by the bacterial surface display screen encode proteins that are either attached to the surface of the respective bacterium and/or are secreted. This is in accordance with the expected role of surface attached or secreted proteins in virulence of the enteric pathogens.

### Example 4

### Gene Distribution Studies with Highly Immunogenic Proteins Identified from Enteroaggregative or Enterotoxigenic E. coli, S. flexneri 2a and C. jejuni NCTC11168

Experimental Procedures

Gene distribution of antigens by PCR. An ideal vaccine antigen would be an antigen that is present in all, or the vast majority of strains of the target organism to which the vaccine is directed. In order to establish whether the genes encoding the identified enteroaggregative or enterotoxigenic E. coli, S. flexneri 2a and C. jejuni NCTC11168 antigens occur ubiquitously in the relevant strains, PCR was performed on a series of independent bacterial isolates with primers specific for the gene of interest Enteroaggregative or enterotoxigenic E. coli, S. flexneri 2a and C. jejuni NCTC11168 isolates were obtained covering the serotypes most frequently present in patients as shown in FIG. 4A. Oligonucleotide sequences as primers were designed for all identified ORFs yielding products of approximately 1,000 bp, if possible covering all identified immunogenic epitopes. Genomic DNA of all Enteroaggregative or enterotoxigenic E. coli, S. flexneri 2a and C. jejuni NCTC11168 strains was prepared as described under Example 2. PCR was performed in a reaction volume of 25 μl using Taq polymerase (1U), 200 nM dNTPs, 10 pMol of each oligonucleotide and the kit according to the manufacturers instructions (Invitrogen, The Netherlands). As standard, 30 cycles (1×: 5 min. 95° C., 30×: 30 sec. 95° C., 30 sec. 56° C., 30 sec. 72° C., 1× 4 min. 72° C.) were performed, unless conditions had to be adapted for individual primer pairs.

Results

Identified genes encoding immunogenic proteins were tested by PCR for their presence in 46 different strains of enteroaggregative or enterotoxigenic E. coli, and S. flexneri, repectively (FIG. 4A). As an example, FIG. 4B shows the PCR reaction for the S. flexneri pCP0179 antigen with all indicated 46 strains. As dearly visible, the gene is present in all strains analysed.

For the two genes as shown in FIGS. 4C and D, the analysis showed that both of these genes from enteroaggregative and enterotoxigenic E. coli, repectively are contained in most of the strains applied for this study. It must be anticipated that the PCR analysis is not capable of detected the same gene in every strain, since only small changes in sequence may abolish amplification by specific primers. Therefore the PCR analysis will only reveal the minimal number of strains that contain the antigen in question. All results with the selected antigens are summarized in Table 5. Importantly, some of the identified antigens are well conserved in all strains of the respective pathogen in sequence and size and are therefore novel vaccine candidates to prevent infections by enteric pathogens.

### Example 5

### Characterization of Immune Sera Obtained from Mice Immunized with Highly Immunogenic Proteins/Peptides from Enteroaggregative or Enterotoxigenic E. coli, S. flexneri 2a or C. jejuni NCTC11168 Displayed on the Surface of E. coli

Experimental Procedures

Generation of Immune Serafrom Mice

E. coli clones harboring plasmids encoding the platform protein fused to a enteroaggregative or enterotoxigenic E. coli, S. flexneri 2a or C. jejuni NCTC11168 peptide, were grown in LB medium supplemented with 50 μg/ml Kanamycin at 37° C. Overnight cultures were diluted 1:10, grown until an OD₆₀₀ of 0.5 and induced with 0.2 mM IPTG for 2 hours. Pelleted bacterial cells were suspended in PBS buffer and disrupted by sonication on ice, generating a crude cell extract. According to the OD₆₀₀ measurement, an aliquot corresponding to 5×10⁷ cells was injected into NMRI mice i.v., followed by a boost after 2 weeks. Serum was taken 1 week after the second injection. Epitope specific antibody levels were measured by peptide ELISA.

Results

Immunogenicity in mice. The presence of specific antibodies was determined by peptide ELISA as it is exemplified in FIG. 5, and summarized in Table 6. Fourty-one antigens from enteroaggregative or enterotoxigenic E. coli, S. flexneri 2a represented by 43 different epitope regions were shown to be immunogenic in mice. These experiments confirmed the bioinformatic prediction that many of the identified epitopes/proteins are immunogenic not only in humans, but also in experimental animals.

### Example 6

### Validation of Peptides from Enteroaggregative or Enterotoxigenic E. coli, S. flexneri 2a or C. jejuni NCTC11168 by Peptide ELISA

Enzynze Linked Immune Assay (ELISA).

ELISA plates (Maxisorb, Millipore) were coated with 5-10 μg/ml total protein diluted in coating buffer (0.1M sodium carbonate pH 9.2). Two dilutions of sera (400×, 2,000×) were made in PBS-BSA. Highly specific Horse Radish Peroxidase (HRP)-conjugated anti-human IgG secondary antibodies (Southern Biotech) were used according to the manufacturers' recommendations (dilutionr 1,000×). Antigen-antibody complexes were quantified by measuring the conversion of the substrate (ABTS) to colored product based on OD_{405 nm} readings by automatic ELISA reader (TECAN SUNRISE). The measurements at 400× dilution were used for the calculation of the results as displayed in Tables 7A-D.

Results

Immunogenicity in humans. The presence of specific antibodies in human sera was determined by peptide ELISA as summarized in Tables 7A-D. The human sera used for this analysis correspond to those that were included in the various serum pools applied for the identification of antigens by the bacterial surface display screens. Single or multiple peptides from individual antigens from enteroaggregative or enterotoxigenic E. coli, S. flexneri 2a and C. jejuni were analysed and many of these were shown to be immunogenic in humans. It is evident that some of the selected peptides are highly reactive with many or all of the investigated human sera (e.g. ECAA005.1, ECC2336.3, ECC4393.3), while others showed intermediate reactivities. For those antigens for which the selected epitope encompassed more than 30 amino adds, multiple peptides were designed with an overlap of 5 to 6 amino adds. For some of the antigens, it was observed that these multiple peptides from the same antigen showed different reactivities, further delineating the immunogenic region of the respective antigen (e.g. SF01383.1-4; ECO3832.1-3). These experiments confirmed that many of the identified epitopes/proteins are highly immunogenic in humans, indicating that they are expressed by the pathogen during infection and capable of inducing a strong immune response.

TABLE 1
Immunogenic proteins identified from E. coli by bacterial surface display.

| | | | No. of | Location of | | |
|---|---|---|---|---|---|---|
| | | | selected | identified | | Seq. |
| E. coli | | | clones per | immunogenic | Seq. | ID |
| antigenic | Putative function | | ORF and | region | ID | (Pro- |
| protein | (by homology) | predicted immunogenic aa* | screen | (aa) | (DNA) | tein) |
| ECs0014 | heat shock protein DnaK | 12-19, 24-29, 37-43, 47-53, 65-72, 83-95, | D: 4, M: 29, | 192-333 | 1 | 301 |
| | | 112-122, 136-147, 162-168, 174-181, | Q: 2 | | | |
| | | 189-195, 201-208, 216-221, 234-243, | | | | |
| | | 270-276, 278-288, 305-316, 318-342, | | | | |
| | | 350-356, 368-400, 420-428, 434-443, | | | | |
| | | 471-477, 481-488, 530-535, 540-547, | | | | |
| | | 566-575, 591-601, 603-609, 624-629 | | | | |
| ECs0041 | l-carnitine dehydratase | 9-22, 38-46, 51-61, 66-73, 108-126, | E: 5 | 69-88 | 2 | 302 |
| | | 136-154, 162-169, 177-186, 198-204, | | | | |
| | | 231-254, 256-272, 277-295, 297-311, | | | | |
| | | 314-328, 331-338, 379-385, 393-402 | | | | |
| ECs0063 | probable ATP-dependent | 18-36, 43-53, 80-86, 94-110, 112-118, | P: 2 | 134-148; 525-552; | 3 | 303 |
| | RNA helicase | 152-168, 170-182, 188-198, 200-220, | | 821-920 | | |
| | | 225-230, 237-243, 248-259, 265-289, | | | | |
| | | 298-317, 325-331, 338-344, 349-360, | | | | |
| | | 382-389, 400-407, 413-419, 433-453, | | | | |
| | | 494-501, 503-524, 530-536, 557-565, | | | | |
| | | 574-582, 586-592, 603-629, 631-637, | | | | |
| | | 643-657, 673-680, 699-705, 715-720, | | | | |
| | | 737-754, 764-771, 778-793, 800-844, | | | | |
| | | 853-858, 874-893, 899-905, 915-929, | | | | |
| | | 957-965 | | | | |
| ECs0067 | L-ribulokinase | 11-22, 28-34, 40-45, 65-86, 99-107, | L: 13 | 251-323; | 4 | 304 |
| | | 115-125, 132-141, 143-150, 158-190, | | 368-389 | | |
| | | 203-211, 216-239, 246-257, 259-270, | | | | |
| | | 272-279, 286-306, 313-332, 338-364, | | | | |
| | | 369-380, 387-397, 410-418, 422-435, | | | | |
| | | 449-455, 467-510, 515-521, 532-538, | | | | |
| | | 547-563 | | | | |
| ECs0089 | meso-diaminopimelate- | 7-20, 28-45, 51-66, 81-104, 108-115, | L: 9 | 235-269 | 5 | 305 |
| | adding enzyme | 124-137, 149-155, 161-206, 209-214, | | | | |
| | | 222-239, 250-262, 276-282, 309-343, | | | | |
| | | 351-363, 365-386, 405-413, 435-440, | | | | |
| | | 446-454, 458-466, 470-477, 482-492 | | | | |
| ECs0152 | helicase, ATP-dependent | 12-43, 51-60, 65-74, 76-86, 102-108, | C: 2 | 752-825 | 6 | 306 |
| | | 110-118, 129-139, 146-160, 164-172, | | | | |
| | | 180-192, 195-208, 212-220, 228-250, | | | | |
| | | 252-267, 271-277, 281-288, 296-313, | | | | |
| | | 344-353, 364-379, 381-387, 394-414, | | | | |
| | | 435-443, 451-460, 468-474, 484-491, | | | | |
| | | 500-510, 541-556, 560-586, 604-618, | | | | |
| | | 635-641, 647-657, 668-705, 715-727, | | | | |
| | | 729-734, 740-745, 760-780, 803-809 | | | | |
| ECs0153 | peptidoglycan synthetase | 16-23, 66-90, 98-110, 125-131, 144-150, | A: 1 | 623-653 | 7 | 307 |
| | MrcB | 194-200, 213-219, 221-232, 237-256, | | | | |
| | | 263-281, 293-298, 311-318, 326-337, | | | | |
| | | 339-354, 373-389, 396-402, 404-421, | | | | |
| | | 427-439, 441-448, 452-462, 467-479, | | | | |
| | | 508-530, 534-541, 544-550, 562-569, | | | | |
| | | 575-581, 583-592, 595-628, 636-656, | | | | |
| | | 658-672, 674-680, 687-697, 715-721, | | | | |
| | | 731-736, 739-749, 754-761, 771-788, | | | | |
| | | 790-797, 813-824 | | | | |
| ECs0155 | ATP-binding component | 14-42, 51-57, 66-77, 84-96, 103-111, | B: 2 | 31-133 | 8 | 308 |
| | of hydroxymate- | 129-148, 158-193, 198-208, 212-222, | | | | |
| | dependent iron transport | 242-262 | | | | |
| ECs0156 | ferrichrome-iron transport | 4-23, 36-62, 65-84, 98-104, 128-135, | C: 4 | 119-152 | 9 | 309 |
| | protein fhuD | 144-161, 175-204, 219-240, 250-264, | | | | |
| | | 266-278, 280-290 | | | | |
| ECs0174 | ribosome releasing factor | 13-26, 33-45, 50-60, 75-81, 97-105, | A: 2, B: 2 | 66-170 | 10 | 310 |
| | | 123-131, 138-145, 158-166, 168-177 | | | | |
| ECs0218 | IcmF-like protein | 20-26, 35-50, 52-67, 85-100, 106-149, | C: 9 | 881-924 | 11 | 311 |
| | | 189-199, 202-208, 217-226, 236-244, | | | | |
| | | 270-294, 310-332, 340-347, 350-356, | | | | |
| | | 364-373, 375-380, 401-428, 438-445, | | | | |
| | | 477-493, 513-548, 555-564, 568-596, | | | | |
| | | 600-612, 650-665, 667-674, 680-687, | | | | |
| | | 696-707, 715-722, 728-764, 779-784, | | | | |
| | | 795-809, 813-820, 837-843, 858-864, | | | | |
| | | 885-891, 894-900, 907-917, 922-935, | | | | |
| | | 941-946, 970-977, 979-986, 1022-1032 | | | | |
| ECs0314 | hypothetical protein | 13-21, 48-57, 72-83, 105-119, 125-133, | A: 1 | 277-306 | 12 | 312 |
| | | 146-153, 170-177, 221-239, 245-274, | | | | |
| | | 283-292, 299-305, 317-329, 335-343, | | | | |
| | | 358-367, 374-380, 399-407, 430-438, | | | | |
| | | 449-454, 473-479, 483-505, 517-527, | | | | |
| | | 531-537, 554-560, 586-599, 601-616, | | | | |
| | | 623-629, 639-647, 649-654, 658-667, | | | | |
| | | 669-676, 690-709, 714-729 | | | | |
| ECs0315 | hypothetical protein | 14-28, 34-40, 45-54, 69-83, 86-100, | L: 3 | 104-138 | 13 | 313 |
| | | 116-123, 135-143, 146-161, 168-179, | | | | |
| | | 187-200, 203-225, 237-250, 255-265, | | | | |
| | | 271-292, 298-314 | | | | |
| ECs0320 | putative receptor | 4-28, 36-42, 78-85, 106-122, 130-135, | A: 1 | 448-483 | 14 | 314 |
| | | 144-150, 161-175, 180-190, 194-200, | | | | |
| | | 226-234, 256-265, 274-294, 309-316, | | | | |
| | | 324-333, 373-379, 382-389, 398-404, | | | | |
| | | 407-416, 422-446, 451-462, 530-541 | | | | |
| ECs0321 | putative enzyme | 5-50, 53-64, 75-80, 110-116, 119-125, | E: 14 | 377-400 | 15 | 315 |
| | | 131-148, 150-156, 192-217, 224-229, | | | | |
| | | 260-267, 275-281, 283-300, 305-310, | | | | |
| | | 323-343, 358-365, 400-406, 422-429, | | | | |
| | | 447-464, 498-503, 512-518, 520-526, | | | | |
| | | 530-537, 552-562, 614-623, 697-703, | | | | |
| | | 705-711, 719-727, 729-743, 745-753, | | | | |
| | | 788-796, 802-810, 813-834 | | | | |
| ECs0322 | hypothetical protein | 4-26, 55-61, 64-70, 74-99, 107-119, | C: 3 | 108-137 | 16 | 316 |
| | | 128-134, 137-154, 167-178 | | | | |
| ECs0336 | putative invasin | 12-54, 70-76, 117-125, 135-143, 196-202, | A: 1 | 894-924 | 17 | 317 |
| | | 205-213, 243-261, 263-269, 278-298, | | | | |
| | | 312-318, 320-326, 334-346, 358-368, | | | | |
| | | 377-389, 396-404, 414-423, 429-438, | | | | |
| | | 448-455, 483-490, 540-549, 557-563, | | | | |
| | | 592-599, 601-609, 622-628, 632-653, | | | | |
| | | 656-692, 695-712, 714-730, 760-766, | | | | |
| | | 775-786, 788-803, 807-814, 820-831, | | | | |
| | | 848-854, 875-886, 892-899, 911-917, | | | | |
| | | 919-925, 927-935, 954-974, 980-992, | | | | |
| | | 1017-1026, 1032-1044, 1074-1079, | | | | |
| | | 1082-1089, 1098-1108, 1115-1120, | | | | |
| | | 1129-1137, 1139-1145, 1161-1170, | | | | |
| | | 1189-1195, 1197-1212, 1219-1225, | | | | |
| | | 1234-1250, 1267-1283, 1302-1318, | | | | |
| | | 1321-1329, 1362-1368, 1377-1388, | | | | |
| | | 1395-1408 | | | | |
| ECs0345 | putative iron-sulfur | 58-76, 82-87, 95-103, 107-114, 122-136, | E: 2 | 2-20 | 18 | 318 |
| | protein | 139-148, 150-162, 165-172, 184-190, | | | | |
| | | 203-220, 228-238, 245-258, 260-267, | | | | |
| | | 288-295, 299-328, 333-352, 357-386, | | | | |
| | | 424-429 | | | | |
| ECs0453 | maltodextrin glucosidase | 5-26, 59-66, 68-74, 81-87, 105-116, | G: 2 | 554-596 | 19 | 319 |
| | | 122-232, 144-160, 185-212, 217-223, | | | | |
| | | 228-234, 241-252, 269-274, 291-313, | | | | |
| | | 318-326, 335-342, 352-358, 368-375, | | | | |
| | | 397-404, 411-422, 431-439, 458-472, | | | | |
| | | 474-485, 493-499, 509-519, 521-527, | | | | |
| | | 530-558, 563-579, 590-601 | | | | |
| ECs0454 | hypothetical protein | 6-12, 18-28, 39-45, 69-102, 120-128, | H: 15 | 178-285 | 20 | 320 |
| | | 137-147, 164-170, 173-179, 223-230, | | | | |
| | | 236-254, 265-277, 320-326, 350-368, | | | | |
| | | 376-400, 404-416, 441-448, 462-477 | | | | |
| ECs0541 | hypothetical protein | 4-19, 21-47, 52-57, 59-73, 79-86, 88-95, | A: 3, D: 5 | 179-265, | 21 | 321 |
| | | 100-108, 114-129, 136-143, 145-151, | | 1343-1368 | | |
| | | 176-182, 235-242, 248-258, 273-281, | | | | |
| | | 301-308, 310-316, 329-340, 347-354, | | | | |
| | | 363-380, 384-400, 407-415, 430-441, | | | | |
| | | 469-480, 491-504, 507-526, 530-540, | | | | |
| | | 547-554, 563-579, 609-617, 620-626, | | | | |
| | | 630-636, 643-655, 665-680, 706-714, | | | | |
| | | 718-725, 729-740, 747-754, 756-779, | | | | |
| | | 790-803, 806-816, 818-824, 829-840, | | | | |
| | | 842-853, 862-877, 901-912, 928-939, | | | | |
| | | 941-952, 961-978, 988-999, 1026-1037, | | | | |
| | | 1067-1078, 1080-1087, 1089-1098, | | | | |
| | | 1104-1115, 1117-1124, 1128-1139, | | | | |
| | | 1143-1151, 1155-1176, 1180-1186, | | | | |
| | | 1205-1211, 1218-1224, 1228-1242, | | | | |
| | | 1244-1251, 1258-1278, 1280-1287, | | | | |
| | | 1290-1298, 1304-1326, 1331-1341, | | | | |
| | | 1363-1378, 1392-1399, 1407-1415, | | | | |
| | | 1430-1443, 1445-1454 | | | | |
| ECS0548 | adhesin/invasin-like | 4-24, 31-37, 61-75, 83-89, 94-102, 117-123, | H: 2 | 238-319 | 22 | 322 |
| | protein | 130-143, 184-191, 203-210, 212-228, | | | | |
| | | 270-284, 286-292, 301-307, 312-319, | | | | |
| | | 329-335 | | | | |
| ECs0557 | putative ATP-binding | 4-16, 22-35, 39-44, 50-59, 65-73, 86-104, | E: 2 | 102-120 | 23 | 323 |
| | component of a transport | 108-117, 128-137, 139-147, 153-159, | | | | |
| | system | 165-171, 185-192, 198-223 | | | | |
| ECs0565 | putative transcriptional | 8-16, 23-30, 32-40, 45-50, 61-68, 81-89, | P: 2 | 265-282 | 24 | 324 |
| | regulator LYSR-type | 91-114, 121-129, 131-149, 161-185, | | | | |
| | | 191-200, 217-224, 229-249, 253-262, | | | | |
| | | 266-273, 282-289, 297-303 | | | | |
| ECs0594 | putative outer membrane | 10-48, 64-71, 81-88, 100-112, 130-140, | P: 3 | 178-193 | 25 | 325 |
| | protein | 153-170, 177-184, 197-202, 236-250, | | | | |
| | | 266-272, 284-292, 294-300, 306-318, | | | | |
| | | 320-326, 346-357, 379-387, 389-396, | | | | |
| | | 405-416, 424-435, 447-453, 474-483, | | | | |
| | | 501-510, 529-536, 550-568, 582-594, | | | | |
| | | 606-611, 625-631, 633-645, 664-672, | | | | |
| | | 685-692, 703-711, 730-745, 761-775, | | | | |
| | | 782-790, 792-804, 816-825, 827-834, | | | | |
| | | 840-866 | | | | |
| ECs0600 | bacteriophage N4 | 11-25, 39-57, 69-94, 100-107, 118-155, | C: 10, G: 6, H: 3 | 88-167, 804-839, | 26 | 326 |
| | adsorption protein NfrA | 158-171, 189-201, 226-233, 236-245, | | | | |
| | | 249-263, 268-277, 287-312, 315-329, | | | | |
| | | 333-342, 351-357, 364-374, 382-388, | | | | |
| | | 399-407, 419-449, 454-471, 486-492, | | | | |
| | | 494-504, 515-541, 547-552, 578-600, | | | | |
| | | 611-623, 625-641, 651-657, 678-692, | | | | |
| | | 699-709, 713-720, 746-752, 772-781, | | | | |
| | | 791-801, 829-844, 880-893, 900-910, | | | | |
| | | 915-923, 936-942, 953-970 | | | | |
| ECs0602 | H repeat-associated | 6-16, 19-40, 55-61, 67-92, 103-108, | E: 8 | 281-297 | 27 | 327 |
| | protein | 125-142, 154-162, 190-196, 236-247, | | | | |
| | | 257-272, 280-290, 310-320, 331-343, | | | | |
| | | 367-375 | | | | |
| ECs0605 | Rhs core protein with | 42-48, 63-116, 151-172, 181-187, 189-196, | E: 52 | 717-741 | 28 | 328 |
| | extension | 200-205, 217-232, 256-261, 288-319, | | | | |
| | | 326-352, 360-374, 380-404, 412-435, | | | | |
| | | 453-466, 474-485, 500-507, 514-519, | | | | |
| | | 525-530, 558-564, 568-582, 590-595, | | | | |
| | | 600-610, 633-640, 666-671, 694-702, | | | | |
| | | 715-722, 730-737, 757-768, 774-785, | | | | |
| | | 820-827, 832-848, 873-878, 883-890, | | | | |
| | | 906-915, 918-930, 937-944, 948-956, | | | | |
| | | 960-966, 970-978, 1023-1040, | | | | |
| | | 1049-1056, 1065-1071, 1085-1090, | | | | |
| | | 1105-1117, 1122-1132, 1165-1171, | | | | |
| | | 1186-1195, 1210-1216, 1309-1342, | | | | |
| | | 1345-1352, 1354-1360, 1375-1385, | | | | |
| | | 1400-1407, 1414-1421, 1430-1439, | | | | |
| | | 1446-1467, 1479-1485, 1522-1530, | | | | |
| | | 1565-1572, 1577-1586, 1596-1608 | | | | |
| ECs0689 | putative dnaK protein | 5-11, 17-24, 26-32, 36-43, 50-61, 67-73, | C: 9, D: 7, E5 | 36-133 | 29 | 329 |
| | | 91-102, 111-126, 133-148, 154-161, | | | | |
| | | 167-173, 179-195, 208-223, 230-252, | | | | |
| | | 270-286, 292-306, 308-347, 352-371, | | | | |
| | | 373-380, 386-395, 404-410, 418-431, | | | | |
| | | 436-444, 447-460, 463-477, 486-492, | | | | |
| | | 522-533, 545-553 | | | | |
| ECs0776 | peptidoglycan-associated | 4-23, 68-78, 100-107, 135-149, 152-159 | F: 15 | 1-88 | 30 | 330 |
| | lipoprotein | | | | | |
| ECs0819 | endolysin | 5-12, 18-27, 35-55, 68-95, 100-109, | B: 2 | 37-98 | 31 | 331 |
| | | 117-122, 129-135, 157-162 | | | | |
| ECs0855 | putative enzyme BioC | 5-52, 64-80, 86-106, 108-155, 175-190, | P: 2 | 53-70 | 32 | 332 |
| | | 223-231, 234-248 | | | | |
| ECs0872 | putative ATP-binding | 25-46, 59-64, 69-75, 83-90, 93-100, | C: 2, E: 3, G: 5, | 226-275 | 33 | 333 |
| | component of a transport | 107-115, 124-135, 151-177, 183-189, | N: 4 | | | |
| | system | 194-206, 209-215, 219-224, 251-263, | | | | |
| | | 267-276, 305-311, 318-327, 332-338, | | | | |
| | | 350-356, 380-396, 406-412, 414-423, | | | | |
| | | 431-437, 453-461, 463-481, 483-491, | | | | |
| | | 505-510, 513-523, 528-545, 568-575 | | | | |
| ECs0880 | hypothetical protein | 5-29, 37-43, 47-54, 61-70 | E: 3 | 31-65 | 34 | 334 |
| ECs0883 | putative outer membrane | 10-35, 42-59, 65-70, 76-85, 92-104, | A: 1 | 547-572 | 35 | 335 |
| | receptor for iron | 149-155, 184-191, 234-243, 248-259, | | | | |
| | transporter | 268-277, 383-389, 391-398, 410-430, | | | | |
| | | 445-454, 488-504, 518-523, 530-538, | | | | |
| | | 574-590, 615-623, 627-633, 652-660, | | | | |
| | | 662-670, 674-683, 703-714, 720-728, | | | | |
| | | 731-737, 751-757 | | | | |
| ECs0931 | modulator of drug activity A | 5-12, 39-51, 57-64, 67-84, 86-108, 124-130, | A: 1, H: 2 | 133-214 | 36 | 336 |
| | | 138-159, 167-179, 181-202, 226-235 | | | | |
| Ecs0954 | putative epimerase or | 12-20, 29-40, 57-77, 79-88, 97-103, | A: 2 | 23-54 | 37 | 337 |
| | dehydratase (lies outside | 111-117, 119-137, 174-200, 202-218, | | | | |
| | the ORF) | 221-229, 231-238, 240-246, 254-264, | | | | |
| | | 266-280, 296-308, 321-331 | | | | |
| ECs0987 | putative formate | 7-17, 19-54, 66-101, 114-133, 146-182, | C: 4 | 1-31 | 38 | 338 |
| | transporter FocA | 193-210, 219-226, 232-238, 244-250, | | | | |
| | | 253-261, 266-279 | | | | |
| ECs1044 | hypothetical protein | 4-11, 17-32, 38-58, 68-111, 113-130, | A: 1 | 661-695 | 39 | 339 |
| | | 132-186, 200-212, 219-227, 240-249, | | | | |
| | | 256-265, 270-278, 285-305, 311-317, | | | | |
| | | 328-341, 343-351, 373-386, 389-414, | | | | |
| | | 419-433, 439-504, 510-535, 553-564, | | | | |
| | | 588-594, 599-604, 609-618, 620-631, | | | | |
| | | 635-657, 664-670, 684-703, 705-717 | | | | |
| ECs1140 | hypothetical protein | 5-33, 67-78, 122-129, 141-150, 172-185, | E: 2, | 70-85, 271-403 | 40 | 340 |
| | | 201-209, 217-223, 235-252, 289-295, | | | | |
| | | 303-316, 355-368, 383-389, 398-406, | | | | |
| | | 426-437, 445-451, 459-467, 479-496, | | | | |
| | | 512-517, 523-530, 535-562, 577-584, | | | | |
| | | 590-605, 610-616, 618-632, 644-654, | | | | |
| | | 663-669, 680-688 | | | | |
| ECs1392 | hypothetical protein | 4-53, 55-62 | E: 2 | 59-71 | 41 | 341 |
| ECs1433 | hypothetical protein | 26-38, 43-63, 67-76, 78-98, 105-112, | G: 3 | 231-283 | 42 | 342 |
| | | 115-121, 132-144, 148-153, 179-184, | | | | |
| | | 194-203, 239-245, 261-278, 282-315 | | | | |
| ECs1462 | RNase E | 13-22, 24-30, 49-61, 65-72, 90-97, 99-105, | C: 1, D: 6 | 571-665 | 43 | 343 |
| | | 115-131, 152-160, 165-171, 176-188, | | | | |
| | | 202-221, 231-250, 255-274, 280-286, | | | | |
| | | 288-296, 331-337, 339-347, 350-358, | | | | |
| | | 374-385, 391-408, 418-427, 438-453, | | | | |
| | | 468-476, 482-490, 497-506, 526-532, | | | | |
| | | 534-583, 696-702, 713-719, 730-748, | | | | |
| | | 750-758, 762-776, 802-808, 825-857, | | | | |
| | | 864-950, 963-1004, 1015-1023, | | | | |
| | | 1046-1058 | | | | |
| ECs1638 | minor tail protein | 5-13, 18-24, 29-45, 51-58, 78-85, 87-94, | P: 3 | 1-83 | 44 | 344 |
| | | 109-117, 122-128, 146-161, 175-189 | | | | |
| ECs1643 | tail length tape measure | 5-17, 40-46, 50-65, 73-86, 89-98, 114-139, | B: 9, | 204-283, 287-363 | 45 | 345 |
| | protein precursor | 151-157, 165-173, 186-195, 197-213, | | | | |
| | | 215-227, 245-260, 310-315, 364-369, | | | | |
| | | 405-415, 428-436, 456-464, 471-482, | | | | |
| | | 507-514, 518-531, 539-565, 648-654, | | | | |
| | | 681-687, 690-707, 720-731, 743-756, | | | | |
| | | 764-771, 801-806, 815-828, 830-836 | | | | |
| ECs1646 | tail assembly protein | 6-27, 33-40, 62-78, 83-89, 91-99, 102-109, | E: 2 | 109-127 | 46 | 346 |
| | | 115-134, 150-156, 160-170, 172-204, | | | | |
| | | 233-244 | | | | |
| ECs1650 | putative tail fiber protein | 4-11, 17-25, 31-38, 55-80, 105-113, | A: 3, M: 2 | 192-275, 394-430 | 47 | 347 |
| | | 115-123, 160-171, 195-201, 210-222, | | | | |
| | | 254-264, 290-296, 301-310, 315-321, | | | | |
| | | 329-334, 349-358, 377-394, 403-409, | | | | |
| | | 424-432, 452-460, 466-474, 480-495, | | | | |
| | | 510-516, 527-539, 554-562, 568-579, | | | | |
| | | 587-592, 599-608, 627-635, 637-646, | | | | |
| | | 661-668, 702-709, 723-736, 744-766, | | | | |
| | | 768-778, 785-802, 815-821, 828-835, | | | | |
| | | 862-868, 876-888, 892-906, 908-918, | | | | |
| | | 921-950 | | | | |
| ECs1736 | hypothetical protein | 18-26, 33-78, 80-87, 120-128, 141-182, | C: 2 | 208-230 | 48 | 348 |
| | | 184-208, 251-257, 269-300, 305-311 | | | | |
| ECs1752 | energy transducer TonB | 11-72, 84-91, 99-109, 112-120, 143-155, | M: 24, H: 7, | 30-176 | 49 | 349 |
| | | 162-183, 188-197, 222-231 | | | | |
| ECs1905 | putative carboxypeptidase | 4-17, 41-56, 61-67, 74-109, 142-149, | G: 7, N: 5 | 84-162 | 50 | 350 |
| | | 158-185, 193-210, 216-236, 241-249 | | | | |
| ECs2026 | methyl-accepting | 8-45, 135-140, 176-182, 189-196, 206-216, | H: 2 | 104-180 | 51 | 351 |
| | chemotaxis protein III | 218-235, 260-269, 272-278, 307-313, | | | | |
| | | 331-344, 352-359, 371-395, 403-414, | | | | |
| | | 416-422, 426-438, 451-470, 478-484, | | | | |
| | | 493-502, 504-511, 514-525, 527-534, | | | | |
| ECs2036 | hypothetical protein | 6-25, 49-59, 65-94, 107-115, 117-124, | G: 8 | 145-173 | 52 | 352 |
| | | 135-151, 176-185, 203-209 | | | | |
| ECs2037 | hypothetical protein | 5-15, 46-56, 58-81, 83-111, 118-138, | G: 4 | 9-46 | 53 | 353 |
| | | 146-158, 165-175 | | | | |
| ECs2044 | putative transport protein | 7-15, 36-43, 54-60, 65-73, 88-94, 107-113, | A: 3 | 100-141 | 54 | 354 |
| | | 122-128, 134-141, 162-171, 182-216, | | | | |
| | | 218-235, 249-258, 266-278, 290-301, | | | | |
| | | 308-338, 362-368 | | | | |
| ECs2048 | putative aldehyde | 4-14, 19-24, 27-36, 38-51, 59-73, 90-96, | L: 3 | 327-355 | 55 | 355 |
| | dehydrogenase | 102-121, 138-150, 157-174, 176-202, | | | | |
| | | 212-225, 229-241, 250-258, 261-268, | | | | |
| | | 279-291, 293-310, 319-338, 358-368, | | | | |
| | | 371-389, 393-398, 404-413, 416-433, | | | | |
| | | 435-442, 458-471 | | | | |
| ECs2062 | H repeat-associated | 10-15, 32-49, 61-69, 97-103, 128-134, | E: 8 | 99-124 | 56 | 356 |
| | protein | 143-154, 164-179 | | | | |
| ECs2091 | putative hemin-binding | 6-37, 40-48, 56-68, 108-127, 135-141, | A: 1 | 434-465 | 57 | 357 |
| | lipoprotein | 145-163, 170-179, 207-216, 224-234, | | | | |
| | | 238-244, 249-261, 266-283, 352-363, | | | | |
| | | 371-378, 380-392, 447-454, 468-489, | | | | |
| | | 497-503, 505-510 | | | | |
| ECs2099 | putative peptidase | 6-33, 46-68, 78-84, 120-131, 135-142, | E: 2 | 406-428 | 58 | 358 |
| | | 183-188, 201-219, 227-233, 236-244, | | | | |
| | | 246-252, 282-295, 307-315, 335-350, | | | | |
| | | 392-399, 409-414, 427-433, 435-447, | | | | |
| | | 468-482, 487-494, 500-506, 529-537, | | | | |
| | | 543-550, 555-575, 577-584, 606-611, | | | | |
| | | 619-625, 637-651, 682-711, 713-727, | | | | |
| | | 729-738, 756-764, 783-795, 801-815, | | | | |
| | | 817-830, 833-847, 861-890, 898-904, | | | | |
| | | 909-928 | | | | |
| ECs2178 | putative head-to-tail | 7-17, 36-53 | P: 14 | 41-65 | 59 | 359 |
| | joining protein | | | | | |
| ECs2330 | putative aminotransferase | 4-10, 17-24, 28-34, 42-49, 55-61, 70-106, | N: 2 | 258-268 | 60 | 360 |
| | | 112-127, 135-142, 154-172, 178-184, | | | | |
| | | 187-201, 213-219, 225-230, 235-246, | | | | |
| | | 253-260, 267-302, 318-331, 339-353, | | | | |
| | | 363-373, 376-387 | | | | |
| ECs2362 | probable ATP-dependent | 8-17, 29-43, 45-52, 58-69, 87-100, 102-111, | G: 6 | 611-689 | 61 | 361 |
| | helicase Lhr | 148-163, 172-187, 190-208, 210-227, | | | | |
| | | 232-239, 245-253, 258-263, 286-299, | | | | |
| | | 313-334, 346-362, 373-388, 391-411, | | | | |
| | | 425-430, 434-446, 457-489, 496-502, | | | | |
| | | 518-524, 537-546, 555-560, 602-610, | | | | |
| | | 637-646, 676-689, 698-704, 706-742, | | | | |
| | | 750-778, 780-791, 806-842, 864-879, | | | | |
| | | 881-888, 890-899, 901-908, 910-921, | | | | |
| | | 941-947, 953-959, 967-980, 990-995, | | | | |
| | | 1000-1061, 1073-1079, 1081-1092, | | | | |
| | | 1096-1118, 1121-1185, 1195-1209, | | | | |
| | | 1219-1232, 1237-1243, 1250-1274, | | | | |
| | | 1276-1282, 1302-1317, 1324-1333, | | | | |
| | | 1339-1344, 1349-1361, 1370-1376, | | | | |
| | | 1406-1413, 1415-1427, 1433-1450, | | | | |
| | | 1453-1469, 1473-1478, 1482-1495, | | | | |
| | | 1509-1517, 1519-1526 | | | | |
| ECs2364 | putative lipoprotein | 7-29, 74-83, 101-107, 115-124, 127-142, | E: 2 | 40-60 | 62 | 362 |
| | | 166-184, 209-215, 222-232, 245-252, | | | | |
| | | 255-262 | | | | |
| ECs2389 | putative ATP-binding | 4-14, 16-32, 42-47, 65-71, 82-109, 128-145, | C: 2 | 138-167 | 63 | 363 |
| | component of a transport | 158-171, 177-191, 197-228, 230-236, | | | | |
| | system | | | | | |
| ECs2394 | putative oxidase | 4-22, 47-59, 61-71, 76-82, 96-103, 119-146, | D: 7 | 943-1016 | 64 | 364 |
| | | 165-172, 174-188, 191-202, 214-232, | | | | |
| | | 240-247, 267-273, 278-293, 303-323, | | | | |
| | | 326-340, 348-353, 365-387, 389-398, | | | | |
| | | 405-411, 416-421, 423-451, 453-480, | | | | |
| | | 482-495, 507-512, 518-529, 537-545, | | | | |
| | | 563-570, 581-587, 591-597, 611-624, | | | | |
| | | 626-634, 637-647, 671-692, 694-725, | | | | |
| | | 735-742, 747-768, 786-825, 859-875, | | | | |
| | | 887-894, 897-909, 915-938, 943-954, | | | | |
| | | 967-976, 984-1001, 1006-1015 | | | | |
| ECs2467 | NADP-specific glutamate | 4-14, 24-34, 47-69, 81-90, 98-112, 144-153, | G: 23, N: 5 | 361-403 | 65 | 365 |
| | dehydrogenase | 161-169, 189-196, 202-208, 213-220, | | | | |
| | | 243-249, 256-262, 265-271, 279-285, | | | | |
| | | 299-307, 310-324, 326-345, 356-369, | | | | |
| | | 397-416, 424-429, 432-441 | | | | |
| ECs2491 | putative scaffolding | 4-22, 27-40, 44-54, 77-91, 112-127, | P: 3 | 171-210 | 66 | 366 |
| | protein in the formation of | 155-161, 196-207, 210-216, 228-234 | | | | |
| | a murein-synthesizing | | | | | |
| | holoenzyme | | | | | |
| ECs2527 | mannose-specific PTS | 4-9, 14-23, 50-56, 59-68, 77-102, 111-120, | C: 4 | 113-157 | 67 | 367 |
| | enzyme IIAB | 126-152, 161-167, 174-180, 189-202, | | | | |
| | | 204-228, 237-245, 259-266, 278-285, | | | | |
| | | 300-309 | | | | |
| ECs2540 | carboxy-terminal protease | 6-26, 31-37, 41-47, 62-69, 71-93, 106-119, | Q: 7 | 517-643 | 68 | 368 |
| | for penicillin-binding | 126-136, 167-180, 196-202, 210-216, | | | | |
| | protein 3 | 242-254, 258-264, 272-286, 292-298, | | | | |
| | | 300-309, 312-322, 346-352, 354-380, | | | | |
| | | 385-392, 401-420, 434-449, 451-459, | | | | |
| | | 465-473, 497-514, 555-562, 566-574, | | | | |
| | | 604-612, 642-648, 657-669 | | | | |
| ECs2662 | flagellin | 23-35, 71-77, 94-100, 134-140, 157-168 | B: 2, D: 3, H: 12 | 11-149, 507-557 | 69 | 369 |
| | | 185-191, 228-237, 255-265, 269-283, | | | | |
| | | 310-315, 334-340, 366-392, 395-400, | | | | |
| | | 404-411, 423-428, 434-445, 451-458, | | | | |
| | | 468-478, 496-508, 512-519, 558-563, | | | | |
| | | 565-582 | | | | |
| ECs2670 | hypothetical protein | 14-20, 35-48, 53-63, 71-77, 95-101, | G: 4 | 23-52 | 70 | 370 |
| | | 114-121, 123-130, 144-151, 153-160, | | | | |
| | | 162-170, 187-197, 201-211 | | | | |
| ECs2676 | flagellar hook-basal body | 7-17, 24-44, 63-70, 88-99 | G: 7, L: 3, N: 4, | 1-78 | 71 | 371 |
| | complex protein FliE | | P: 6 | | | |
| ECs2699 | DNA cytosine methylase | 21-39, 46-53, 68-96, 107-113, 118-124, | L: 12, P: 11 | 92-170, 178-199 | 72 | 372 |
| | | 126-135, 158-185, 196-202, 204-213, | | | | |
| | | 219-226, 246-253, 267-275, 277-285, | | | | |
| | | 299-317, 319-338, 404-410, 421-428, | | | | |
| | | 435-463 | | | | |
| Ecs2776 | hypothetical protein | 28-43, 47-55, 59-68, 72-79, 106-112, | E: 2, L: 5 | 403-455 | 73 | 373 |
| | | 121-139, 151-160, 168-175, 177-183, | | | | |
| | | 194-212, 223-229, 232-248, 254-263, | | | | |
| | | 270-276, 317-323, 331-338, 342-356, | | | | |
| | | 363-369, 378-391, 415-424, 432-441, | | | | |
| | | 443-456, 464-470, 499-505, 521-527, | | | | |
| | | 534-552, 586-599, 624-634, 639-647, | | | | |
| | | 651-667, 685-690, 694-702, 711-731, | | | | |
| | | 733-744, 752-776, 784-791, 801-807, | | | | |
| | | 837-859, 879-890, 906-914, 918-924, | | | | |
| | | 926-940, 945-958, 965-971, 980-1002, | | | | |
| | | 1010-1016, 1018-1028, 1034-1044, | | | | |
| | | 1046-1053, 1065-1075, 1079-1092, | | | | |
| | | 1095-1104, 1125-1142, 1154-1162, | | | | |
| | | 1176-1181, 1194-1207, 1233-1244, | | | | |
| | | 1252-1261, 1267-1274, 1283-1288, | | | | |
| | | 1318-1324, 1327-1342 | | | | |
| ECs2865 | hypothetical protein | 17-25, 32-77, 82-91, 100-128, 163-169, | H: 7 | 290-399 | 74 | 374 |
| | | 189-207, 211-218, 227-232, 239-245, | | | | |
| | | 255-260, 278-300, 311-325, 342-356, | | | | |
| | | 382-390, 393-401, 416-460, 467-487, | | | | |
| | | 491-497, 505-512, 516-532, 551-565, | | | | |
| | | 568-575, 594-601, 610-632, 638-643, | | | | |
| | | 647-670, 672-685, 699-710, 712-726 | | | | |
| ECs2882 | putative membrane | 4-39, 56-73, 107-128, 134-142, 144-153, | E: 14 | 177-199 | 75 | 375 |
| | protein | 155-183, 198-203, 205-212, 215-223, | | | | |
| | | 232-244, 248-265, 273-292, 294-301, | | | | |
| | | 304-311, 322-329, 338-343, 369-378, | | | | |
| | | 397-404, 408-416, 420-426, 436-443 | | | | |
| ECs3019 | beta-D-glucoside | 4-22, 25-31, 35-41, 53-61, 74-83, 101-145, | M: 4, Q: 2 | 393-543 | 76 | 376 |
| | glucohydrolase | 157-162, 199-216, 247-257, 266-276, | | | | |
| | | 282-289, 291-298, 306-313, 324-335, | | | | |
| | | 345-353, 360-368, 392-400, 404-421, | | | | |
| | | 432-445, 455-462, 478-486, 494-499, | | | | |
| | | 501-511, 525-551, 554-561, 581-588, | | | | |
| | | 600-613, 637-661, 669-676, 684-697, | | | | |
| | | 699-705, 720-730, 746-751 | | | | |
| ECs3081 | putative ATP-binding | 11-25, 65-79, 89-97, 106-112, 115-121, | C: 6 | 635-686 | 77 | 377 |
| | component of a transport | 126-132, 134-141, 218-230, 255-260, | | | | |
| | system | 287-294, 304-309, 328-334, 339-345, | | | | |
| | | 347-363, 366-382, 421-428, 457-463, | | | | |
| | | 471-477, 484-492, 504-511, 513-518, | | | | |
| | | 547-554, 559-572, 598-604, 617-628, | | | | |
| | | 641-647, 658-665, 691-696, 701-714, | | | | |
| | | 744-751, 760-770, 774-780, 792-801, | | | | |
| | | 805-817 | | | | |
| ECs3103 | hypothetical protein | 5-25, 31-39, 72-79, 93-102, 104-110, | A: 2 | 74-108 | 78 | 378 |
| | | 122-132, 138-146, 157-189, 192-198, | | | | |
| | | 205-214, 226-233, 240-248, 269-275, | | | | |
| | | 282-298, 304-310, 313-327, 342-348 | | | | |
| ECs3111 | putative membrane | 7-34, 44-50, 54-64, 90-99, 101-106, | C: 3 | 1352-1387 | 79 | 379 |
| | protein | 111-123, 156-175, 182-212, 224-232, | | | | |
| | | 235-247, 249-264, 266-273, 306-321, | | | | |
| | | 326-333, 343-351, 359-365, 370-403, | | | | |
| | | 424-455, 466-477, 481-495, 503-511, | | | | |
| | | 516-531, 534-543, 555-573, 578-600, | | | | |
| | | 638-650, 657-671, 677-682, 686-692, | | | | |
| | | 698-710, 721-729, 732-741, 752-763, | | | | |
| | | 773-784, 786-809, 816-821, 829-849, | | | | |
| | | 885-894, 911-920, 931-940, 942-949, | | | | |
| | | 954-962, 979-986, 988-995, 1007-1016, | | | | |
| | | 1034-1039, 1060-1065, 1076-1093, | | | | |
| | | 1131-1137, 1144-1152, 1160-1165, | | | | |
| | | 1170-1181, 1186-1196, 1220-1260, | | | | |
| | | 1271-1280, 1287-1295, 1318-1328, | | | | |
| | | 1346-1356, 1361-1367, 1378-1392, | | | | |
| | | 1401-1407, 1412-1420, 1426-1443, | | | | |
| | | 1478-1489, 1491-1499, 1501-1525 | | | | |
| ECs3114 | DNA gyrase, subunit A, | 18-53, 64-93, 95-105, 124-135, 143-148, | D: 37 | 7-84 | 80 | 380 |
| | type II topoisomerase | 155-161, 163-171, 184-198, 238-245, | | | | |
| | | 258-271, 273-284, 287-292, 302-310, | | | | |
| | | 312-320, 322-341, 349-365, 377-403, | | | | |
| | | 407-414, 417-423, 444-453, 455-469, | | | | |
| | | 471-495, 503-511, 536-557, 579-586, | | | | |
| | | 588-609, 619-626, 632-638, 643-649, | | | | |
| | | 656-663, 669-680, 682-688, 699-714, | | | | |
| | | 729-739, 755-761, 768-776, 781-793, | | | | |
| | | 801-815, 821-826, 833-842, 863-869 | | | | |
| ECs3126 | anaerobic sn-glycerol-3- | 8-15, 24-40, 51-65, 78-89, 102-111, | G: 10 | 1-26 | 81 | 381 |
| | phosphate dehydrogenase | 117-154, 164-177, 181-192, 198-209, | | | | |
| | la | 216-222, 230-237, 241-248, 254-268, | | | | |
| | | 285-293, 298-321, 331-338, 366-373, | | | | |
| | | 379-389, 392-415, 429-439, 441-451, | | | | |
| | | 453-459, 471-486, 489-501, 524-535 | | | | |
| ECs3137 | hypothetical protein | 10-18, 26-38, 48-54, 60-69, 77-83, 88-95, | C: 4 | 64-88 | 82 | 382 |
| | | 119-126, 133-169, 172-185, 193-206, | | | | |
| | | 214-225, 236-250, 255-261, 269-275, | | | | |
| | | 278-301, 320-329, 336-341, 345-353, | | | | |
| | | 356-369, 389-397 | | | | |
| ECs3205 | cell division protein | 27-32, 37-50, 68-82, 84-108, 134-145, | A: 2 | 86-134 | 83 | 383 |
| | | 147-154, 162-170, 172-182, 194-200, | | | | |
| | | 205-224, 232-270, 293-299, 312-328 | | | | |
| ECs3213 | chorismate synthase | 7-13, 18-44, 64-74, 81-86, 94-104, 134-148, | C: 2 | 100-141 | 84 | 384 |
| | | 153-159, 174-183, 204-225, 228-243, | | | | |
| | | 248-255, 283-295, 297-303, 320-347 | | | | |
| ECs3221 | putative outer membrane | 4-27, 36-42, 55-62, 64-73, 92-106, 112-118, | F: 1 | 344-369 | 85 | 385 |
| | protein | 120-127, 135-154, 170-179, 242-257, | | | | |
| | | 270-277, 286-325, 335-341, 359-365, | | | | |
| | | 381-387, 410-440, 470-478, 520-528, | | | | |
| | | 543-553, 575-582, 603-612, 617-623, | | | | |
| | | 628-649, 657-663, 685-691, 693-699, | | | | |
| | | 703-708, 712-719, 740-747, 755-763, | | | | |
| | | 766-782, 800-809, 811-833, 835-851, | | | | |
| | | 856-862, 864-876 | | | | |
| ECs3225 | putative acyltransferase | 4-10, 15-24, 26-53, 55-71, 78-83, 90-113, | A: 1 | 204-232 | 86 | 386 |
| | | 128-148, 156-163, 165-179, 203-213, | | | | |
| | | 228-239, 250-259, 277-285, 292-314, | | | | |
| | | 322-330, 334-340, 345-360, 381-396, | | | | |
| | | 404-409, 416-427 | | | | |
| ECs3264 | hypothetical protein | 4-17, 21-30, 42-49, 56-63, 67-73, 78-87, | N: 2 | 104-126 | 87 | 387 |
| | | 92-99, 105-111, 122-130, 151-160, 168-197, | | | | |
| | | 209-226, 243-276, 286-293, 295-301, | | | | |
| | | 306-319, 322-332, 335-342 | | | | |
| ECs3265 | putative peptidase | 4-10, 12-23, 28-34, 37-60, 65-84, 97-103, | M: 7, Q: 21 | 300-362 | 88 | 388 |
| | | 113-127, 135-143, 182-187, 200-223, | | | | |
| | | 227-233, 236-271, 274-279, 282-287, | | | | |
| | | 293-299, 314-329, 334-358 | | | | |
| ECs3322 | hypothetical protein | 20-32, 37-46, 48-65, 75-83, 86-95, 121-133, | A: 2, G: 7 | 1-38 | 89 | 389 |
| | | 138-151, 183-190, 199-205, 216-227 | | | | |
| ECs3330 | putative oxidoreductase | 9-25, 29-48, 50-100, 102-126, 131-149, | A: 3, | 506-577 | 90 | 390 |
| | Fe—S subunit | 167-173, 210-217, 224-256, 259-270, | | | | |
| | | 275-292, 295-301, 308-313, 319-335, | | | | |
| | | 337-359, 362-382, 393-423, 436-449, | | | | |
| | | 468-476, 481-487, 492-500, 526-534, | | | | |
| | | 537-548, 560-567, 569-579, 590-598, | | | | |
| | | 604-613, 629-636, 644-656 | | | | |
| ECs3378 | putative membrane | 25-45, 53-78, 80-102, 116-128, 161-167, | P: 10 | 214-233 | 91 | 391 |
| | protein | 180-186, 193-219, 235-258, 261-268, | | | | |
| | | 291-318 | | | | |
| ECs3386 | hypothetical protein | 4-18, 25-31, 33-39, 47-53, 64-92, 97-106, | A: 2 | 299-326 | 92 | 392 |
| | | 123-129, 134-146, 165-171, 173-190, | | | | |
| | | 192-213, 226-239, 251-273, 283-298, | | | | |
| | | 316-324, 339-345, 350-356, 361-376, | | | | |
| | | 400-408, 418-440, 444-451, 476-481, | | | | |
| | | 505-516, 524-542, 555-563, 581-594, | | | | |
| | | 607-629, 634-641, 647-670, 711-719, | | | | |
| | | 728-738, 755-765, 772-780, 800-815, | | | | |
| | | 822-833, 842-852, 860-865, 874-880, | | | | |
| | | 891-913, 926-938, 941-946, 961-978, | | | | |
| | | 984-990, 1013-1024, 1052-1092, | | | | |
| | | 1099-1111, 1120-1140, 1153-1168, | | | | |
| | | 1170-1190, 1193-1211, 1221-1233, | | | | |
| | | 1253-1264, 1268-1274, 1283-1289, | | | | |
| | | 1295-1300, 1303-1327, 1338-1351, | | | | |
| | | 1362-1368, 1391-1396, 1403-1416, | | | | |
| | | 1429-1436, 1471-1477, 1483-1513, | | | | |
| | | 1526-1555, 1585-1591, 1596-1630, | | | | |
| | | 1632-1639 | | | | |
| ECs3414 | putative LACI-type | 10-25, 34-54, 57-67, 77-96, 111-121, | G: 3, H: 6 | 156-268 | 93 | 393 |
| | transcriptional regulator | 127-139, 151-157, 161-179, 183-198, | | | | |
| | | 201-219, 233-239, 247-252, 268-276, | | | | |
| | | 283-294, 299-309, 319-324 | | | | |
| ECs3415 | hypothetical protein | 6-28, 34-45, 64-79, 88-95, 98-115, 120-141, | C: 4 | 934-1003 | 94 | 394 |
| | | 159-167, 174-179, 186-192, 198-208, | | | | |
| | | 216-225, 232-246, 248-273, 275-283, | | | | |
| | | 291-299, 304-316, 370-381, 386-393, | | | | |
| | | 401-417, 421-445, 460-468, 470-487, | | | | |
| | | 497-509, 511-535, 542-558, 564-574, | | | | |
| | | 603-609, 619-648, 661-675, 682-694, | | | | |
| | | 720-738, 742-759, 762-788, 793-805, | | | | |
| | | 825-851, 885-893, 898-906, 918-935, | | | | |
| | | 941-953, 971-978, 986-993, 1001-1017, | | | | |
| | | 1019-1026, 1050-1070, 1072-1089, | | | | |
| | | 1097-1102, 1107-1121 | | | | |
| ECs3431 | RecO protein | 6-13, 31-38, 47-60, 71-102, 107-124, | N: 3, P: 10 | 202-243 | 95 | 395 |
| | | 128-155, 173-180, 213-220 | | | | |
| ECs3515 | putative ATP-binding | 4-38, 49-71, 75-85, 110-115, 168-173, | A: 2, C: 1, F: 7, | 454-483, | 96 | 396 |
| | component of a transport | 202-210, 221-228, 240-245, 258-264, | L: 26, M: 6, | 1142-1349 | | |
| | system | 302-316, 348-362, 386-391, 456-462, | P: 4, Q: 13 | | | |
| | | 474-483, 494-499, 511-516, 523-528, | | | | |
| | | 533-539, 549-557, 579-585, 587-593, | | | | |
| | | 618-625, 627-634, 654-660, 664-670, | | | | |
| | | 682-688, 697-702, 729-735, 783-793, | | | | |
| | | 804-812, 817-829, 862-868, 908-920, | | | | |
| | | 954-960, 1000-1006, 1008-1031, 1044-1050, | | | | |
| | | 1069-1077, 1079-1084, 1097-1118, | | | | |
| | | 1139-1146, 1152-1158, 1165-1176, | | | | |
| | | 1181-1186, 1201-1213, 1261-1267, | | | | |
| | | 1272-1280, 1282-1289, 1358-1364, | | | | |
| | | 1373-1382, 1390-1400, 1443-1450, | | | | |
| | | 1497-1505, 1530-1552, 1560-1568 | | | | |
| ECs3597 | L-isoaspartate protein | 4-13, 20-34, 47-53, 58-65, 76-82, 89-106, | L: 2 | 10-41 | 97 | 397 |
| | carboxylmethyltransferase | 139-160, 165-182, 191-205 | | | | |
| ECs3736 | hypothetical protein | 31-42, 59-75, 91-102, 104-123, 147-153, | G: 6 | 4-34 | 98 | 398 |
| | | 172-184, 193-206, 257-266, 306-316, | | | | |
| | | 318-329 | | | | |
| ECs3745 | putative deacetylase | 5-14, 26-35, 38-45, 54-60, 63-79, 121-127, | L: 2, P: 2 | 64-148 | 99 | 399 |
| | | 137-145, 152-162, 167-173, 175-183, | | | | |
| | | 191-202, 218-228, 238-263, 278-295, | | | | |
| | | 303-316, 320-335, 337-345, 359-365, | | | | |
| | | 382-400 | | | | |
| ECs3811 | hypothetical protein | 4-17, 31-39, 46-61, 68-73, 76-97, 128-139, | C: 2 | 187-223 | 100 | 400 |
| | | 150-156, 166-172, 174-182, 184-215, | | | | |
| | | 219-225, 238-245, 249-262 | | | | |
| ECs3821 | DNA-specific | 4-23, 30-41, 44-53, 58-70, 82-91, 107-114, | G: 2 | 1-69 | 101 | 401 |
| | endonuclease I | 122-129, 148-155, 201-207, 223-232 | | | | |
| ECs3824 | hypothetical protein | 4-16, 28-41, 44-52, 60-66, 73-82, 92-101, | A: 3 | 89-130 | 102 | 402 |
| | | 108-114, 133-138, 145-155, 177-185, | | | | |
| | | 194-202 | | | | |
| ECs3827 | putative protein transport | 4-9, 21-39, 72-78, 82-88, 99-131, 136-143, | E: 2, G: 2, P: 3 | 209-237 | 103 | 403 |
| | | 151-162, 164-187, 189-204, 208-216, | | | | |
| | | 223-229, 232-240, 246-256, 269-283, | | | | |
| | | 288-299, 311-321, 328-335 | | | | |
| ECs3832 | putative alpha helix chain | 4-14, 36-48, 66-73, 75-89, 95-103, 115-123, | F: 1 | 178-262 | 104 | 404 |
| | | 128-133, 140-145, 151-158, 165-176, | | | | |
| | | 178-188, 224-254, 267-278, 289-297, | | | | |
| | | 302-311 | | | | |
| ECs3836 | hypothetical protein | 19-25, 55-70, 76-82, 88-107, 114-129, | P: 20, L: 3 | 1-35 | 105 | 405 |
| | | 136-145, 154-177, 205-219, 227-233 | | | | |
| ECs3843 | integrase | 26-33, 39-45, 50-62, 76-85, 87-101, | C: 4, H: 5, P: 3 | 254-315, 323-407 | 106 | 406 |
| | | 116-131, 142-152, 154-186, 193-199, | | | | |
| | | 201-217, 221-243, 266-272, 281-298, | | | | |
| | | 324-330, 335-342, 345-355, 375-383, | | | | |
| | | 407-413 | | | | |
| ECs3923 | outer membrane channel | 4-22, 27-36, 60-69, 90-98, 107-113, | C: 5 | 358-400 | 107 | 407 |
| | TolC | 117-123, 127-134, 137-151, 154-161, | | | | |
| | | 169-178, 185-192, 202-208, 214-223, | | | | |
| | | 230-239, 245-255, 266-275, 307-317, | | | | |
| | | 323-337, 339-353, 361-379, 385-391, | | | | |
| | | 393-401, 415-422, 424-429, 434-442, | | | | |
| | | 444-449, 470-480 | | | | |
| ECs3937 | hypothetical protein | 4-25, 31-42, 83-101, 109-123, 127-136, | N: 5 | 223-239 | 108 | 408 |
| | | 139-145, 154-166, 169-182, 194-201, | | | | |
| | | 210-220, 226-237, 251-275, 277-304, | | | | |
| | | 309-329, 341-362, 367-372, 377-393, | | | | |
| | | 400-406 | | | | |
| ECs3951 | hypothetical protein | 4-22, 29-34, 37-44, 48-78, 98-110, 127-142, | C: 2 | 59-127 | 109 | 409 |
| | | 144-156, 158-165 | | | | |
| ECs4037 | hypothetical protein | 4-12, 14-20, 27-34, 39-47, 51-67, 69-81, | L: 4 | 67-105 | 110 | 410 |
| | | 89-97, 105-119, 121-133, 140-149, 151-161 | | | | |
| ECs4041 | putative enzyme | 6-19, 25-35, 43-48, 56-69, 73-93, 137-146, | E: 3, N: 2 | 259-291 | 111 | 411 |
| | | 152-161, 164-207, 212-229, 236-241, | | | | |
| | | 244-250, 273-288, 292-299, 314-324 | | | | |
| ECs4049 | protein chain initiation | 17-24, 34-40, 78-85, 227-233, 294-315, | D: 3, M: 45, | 80-214 | 112 | 412 |
| | factor IF-2 | 327-335, 345-351, 354-359, 363-368, | Q: 7 | | | |
| | | 388-403, 405-411, 413-419, 425-434, | | | | |
| | | 462-472, 480-500, 528-536, 542-560, | | | | |
| | | 566-573, 579-589, 593-606, 614-646, | | | | |
| | | 651-658, 663-669, 686-726, 734-747, | | | | |
| | | 754-778, 787-806, 809-825, 827-839, | | | | |
| | | 876-887 | | | | |
| ECs4051 | hypothetical protein | 4-9, 15-29, 38-43, 50-81, 83-96, 98-108, | E: 3 | 133-148 | 113 | 413 |
| | | 116-122, 136-143 | | | | |
| ECs4061 | D-alanyl-D-alanine | 5-79, 98-105, 133-146, 158-182, 189-207, | N: 2 | 132-153 | 114 | 414 |
| | carboxypeptidase | 213-225, 231-252, 272-278, 283-303, | | | | |
| | | 312-317, 333-346, 361-367, 370-379, | | | | |
| | | 387-419, 421-433, 439-453, 460-468 | | | | |
| ECs4079 | hypothetical protein | 9-29, 35-40, 49-63, 69-76, 110-134, | C: 2 | 63-101 | 115 | 415 |
| | | 141-147, 160-169 | | | | |
| ECs4084 | hypothetical protein | 4-9, 13-20, 25-43, 50-56, 75-86, 102-115, | N: 3, H: 5 | 54-161, 256-285 | 116 | 416 |
| | | 120-126, 128-135, 139-145, 161-166, | | | | |
| | | 170-189, 202-210, 212-219, 221-232, | | | | |
| | | 240-248, 252-264 | | | | |
| ECs4091 | glutamate synthase large | 7-17, 19-33, 44-51, 56-70, 74-79, 85-93, | B: 2, L: 2 | 1018-1052, | 117 | 417 |
| | subunit | 96-102, 110-117, 124-132, 140-148, | | 1134-1262 | | |
| | | 157-176, 204-248, 256-269, 289-306, | | | | |
| | | 318-324, 331-339, 377-382, 389-397, | | | | |
| | | 399-411, 413-420, 424-432, 436-441, | | | | |
| | | 462-469, 499-506, 522-547, 549-563, | | | | |
| | | 569-575, 578-594, 609-614, 621-630, | | | | |
| | | 637-646, 652-685, 688-711, 713-724, | | | | |
| | | 739-744, 752-783, 793-799, 811-823, | | | | |
| | | 825-841, 846-855, 861-868, 874-886, | | | | |
| | | 895-907, 935-966, 977-1024, 1026-1035, | | | | |
| | | 1037-1055, 1063-1091, 1094-1103, | | | | |
| | | 1105-1119, 1128-1149, 1160-1173, | | | | |
| | | 1182-1194, 1210-1222, 1227-1234, | | | | |
| | | 1244-1258, 1275-1285, 1293-1300, | | | | |
| | | 1316-1322, 1336-1354, 1357-1364, | | | | |
| | | 1367-1372, 1386-1392, 1403-1411, | | | | |
| | | 1424-1430, 1439-1456, 1458-1469, | | | | |
| | | 1485-1504 | | | | |
| ECs4147 | dehydroshikimate | 4-30, 32-42, 59-65, 78-88, 104-127, | D: 2 | 6-90 | 118 | 418 |
| | reductase | 132-147, 158-171, 181-187, 195-214, | | | | |
| | | 220-226, 238-269 | | | | |
| ECs4174 | 50S ribosomal subunit | 10-16, 25-53, 64-74 | H: 6 | 1-83 | 119 | 419 |
| | protein L24 | | | | | |
| ECs4198 | FKBP-type peptidyl-prolyl | 4-29, 48-57, 75-86, 99-113, 146-155, | Q: 2 | 101-185 | 120 | 420 |
| | cis-trans isomerase | 164-174, 190-201, 215-234, 236-251 | | | | |
| ECs4216 | nitrite reductase | 4-9, 32-56, 58-67, 71-81, 90-95, 97-105, | N: 20 | 177-207 | 121 | 421 |
| | (NAD(P)H) subunit | 112-118, 124-132, 138-144, 147-167, | | | | |
| | | 170-177, 211-217, 231-241, 250-258, | | | | |
| | | 260-272, 274-282, 289-296, 299-309, | | | | |
| | | 319-331, 344-350, 356-362, 368-377, | | | | |
| | | 381-394, 399-406, 412-430, 432-450, | | | | |
| | | 459-473, 486-503, 508-515, 520-548, | | | | |
| | | 564-570, 581-587, 616-623, 628-635, | | | | |
| | | 638-660, 678-684, 691-696, 703-709, | | | | |
| | | 716-723, 760-772, 787-795, 835-844 | | | | |
| ECs4233 | putative transport protein | 5-43, 46-81, 88-95, 137-142, 163-191, | L: 3, M: 2, P: 5 | 58-179 | 122 | 422 |
| | | 195-203, 210-235, 241-254, 256-276, | | | | |
| | | 280-288, 292-305, 307-313, 317-333, | | | | |
| | | 335-343, 347-353, 357-363, 372-381, | | | | |
| | | 384-389, 399-409 | | | | |
| ECs4249 | hypothetical protein | 26-32, 38-44, 68-75, 85-100, 104-114, | A: 1, H: 3 | 542-627, 691-724 | 123 | 423 |
| | | 126-132, 140-150, 153-164, 175-193, | | | | |
| | | 200-209, 218-224, 226-232, 243-249, | | | | |
| | | 251-260, 275-293, 304-329, 335-353, | | | | |
| | | 364-479, 485-490, 500-512, 514-523, | | | | |
| | | 532-556, 577-589, 622-628, 631-653, | | | | |
| | | 656-678 | | | | |
| ECs4258 | 4-alpha- | 8-22, 25-30, 46-62, 67-73, 98-103, 105-114, | G: 5 | 170-207 | 124 | 424 |
| | glucanotransferase | 120-141, 144-153, 168-175, 181-193, | | | | |
| | | 198-204, 208-227, 235-242, 249-258, | | | | |
| | | 281-288, 291-306, 327-336, 340-361, | | | | |
| | | 368-380, 389-409, 417-426, 428-435, | | | | |
| | | 442-453, 468-486, 488-496, 498-509, | | | | |
| | | 511-523, 540-553, 566-579, 587-603, | | | | |
| | | 629-636, 677-682 | | | | |
| ECs4337 | hypothetical lipoprotein | 9-25, 41-61, 68-75, 81-102, 106-141, | A: 1 | 32-53 | 125 | 425 |
| | | 158-165, 173-191 | | | | |
| ECs4346 | ATP-binding protein of | 7-26, 28-37, 43-58, 67-79, 92-99, 103-111, | C: 2 | 95-122 | 126 | 426 |
| | nickel transport system | 118-128, 130-139, 152-165, 170-186, | | | | |
| | | 192-214, 216-223, 225-251 | | | | |
| ECs4347 | ATP-binding protein of | 4-16, 21-36, 38-47, 54-64, 92-103, 117-126, | L: 5, P: 2, Q: 2 | 70-163 | 127 | 427 |
| | nickel transport system | 134-155, 157-200, 202-223, 246-262 | | | | |
| ECs4410 | putative oxidoreductase | 30-38, 45-51, 75-90, 93-114, 119-127, | A: 6, H: 3 | 44-131, 484-615 | 128 | 428 |
| | subunit | 134-143, 151-159, 166-180, 189-207, | | | | |
| | | 217-222, 230-239, 267-276, 283-292, | | | | |
| | | 324-350, 374-386, 392-403, 409-416, | | | | |
| | | 418-424, 451-458, 466-477, 483-494, | | | | |
| | | 501-509, 521-528, 540-549, 556-561, | | | | |
| | | 573-579, 581-588, 621-626, 628-636, | | | | |
| | | 654-661, 695-701, 711-717, 734-743, | | | | |
| | | 751-757, 764-771, 789-798, 832-837, | | | | |
| | | 860-867, 869-883, 911-917, 942-950, | | | | |
| | | 958-964, 966-981, 992-999 | | | | |
| ECs4412 | hypothetical protein | 5-14, 27-42, 48-67, 71-83, 85-91, 105-112, | L: 2 | 541-569 | 129 | 429 |
| | | 114-135, 139-147, 159-165, 169-185, | | | | |
| | | 188-195, 199-208, 212-221, 231-253, | | | | |
| | | 264-272, 275-282, 290-303, 309-319, | | | | |
| | | 324-331, 340-358, 380-405, 419-425, | | | | |
| | | 438-444, 450-463, 468-477, 497-514, | | | | |
| | | 520-533, 549-556, 568-574, 617-626, | | | | |
| | | 637-643, 661-668, 674-684, 705-713, | | | | |
| | | 718-733, 735-775 | | | | |
| ECs4413 | putative cellulose | 13-19, 21-32, 45-69, 79-87, 90-109, | C: 3 | 104-137 | 130 | 430 |
| | synthase | 140-148, 156-208, 215-232, 243-274, | | | | |
| | | 276-284, 288-298, 301-316, 339-347, | | | | |
| | | 369-384, 405-412, 430-437, 445-457, | | | | |
| | | 464-470, 475-483, 490-509, 517-524, | | | | |
| | | 532-591, 609-628, 647-677, 681-709, | | | | |
| | | 731-740, 752-767, 770-781, 787-793, | | | | |
| | | 798-807, 825-836, 839-869 | | | | |
| ECs4480 | hypothetical protein | 4-10, 33-44, 73-78, 93-101, 123-129, | L: 2 | 1520-1553 | 131 | 431 |
| | | 135-165, 201-214, 251-261, 268-274, | | | | |
| | | 285-292, 316-322, 328-336, 342-350, | | | | |
| | | 353-360, 374-387, 391-399, 401-406, | | | | |
| | | 417-425, 437-443, 447-454, 511-522, | | | | |
| | | 530-535, 727-737, 762-774, 781-787, | | | | |
| | | 803-809, 827-838, 852-859, 877-883, | | | | |
| | | 901-907, 910-918, 951-956, 996-1002, | | | | |
| | | 1071-1079, 1086-1096, 1098-1104, | | | | |
| | | 1106-1119, 1129-1135, 1142-1148, | | | | |
| | | 1155-1161, 1167-1185, 1199-1205, | | | | |
| | | 1224-1232, 1242-1251, 1279-1287, | | | | |
| | | 1293-1299, 1305-1321, 1372-1396, | | | | |
| | | 1426-1435, 1467-1473, 1479-1492, | | | | |
| | | 1526-1533, 1548-1558, 1578-1585 | | | | |
| ECs4629 | hypothetical protein | 4-10, 36-45, 56-92, 108-114, 125-133, | C: 2, L: 19 | 95-189 | 132 | 432 |
| | | 137-146, 156-162, 164-186, 194-203, | | | | |
| | | 225-234, 242-251, 272-283, 285-306, | | | | |
| | | 310-315, 322-330, 358-371, 373-379, | | | | |
| | | 389-396 | | | | |
| ECs4674 | membrane-bound ATP | 4-32, 38-46, 66-83, 88-95, 110-118, | A: 1 | 51-81 | 133 | 433 |
| | synthase beta-subunit | 123-141, 169-180, 200-208, 217-225, | | | | |
| | AtpD | 237-245, 247-261, 263-272, 275-282, | | | | |
| | | 291-302, 310-338, 345-353, 360-369, | | | | |
| | | 371-378, 386-394, 398-413, 416-422, | | | | |
| | | 437-448 | | | | |
| ECs4691 | ATP-binding component | 4-10, 12-38, 59-64, 81-97, 122-132, | E: 2, N: 4 | 14-27 | 134 | 434 |
| | of D-ribose high-affinity | 136-142, 149-165, 180-187, 192-199, | | | | |
| | transporter | 205-216, 222-228, 244-251, 255-274, | | | | |
| | | 280-286, 294-320, 327-333, 339-346, | | | | |
| | | 353-359, 372-385, 402-408, 413-420, | | | | |
| | | 433-440, 443-453, 456-461, 464-472, | | | | |
| | | 483-495 | | | | |
| ECs4725 | putative cytochrome | 4-40, 42-56, 59-73, 76-110, 115-128, | E: 6 | 239-261 | 135 | 435 |
| | | 132-139, 148-170, 174-195, 197-207, | | | | |
| | | 214-220, 222-236, 238-246, 252-283, | | | | |
| | | 291-326, 334-413 | | | | |
| ECs4761 | uridine phosphorylase | 4-11, 18-26, 31-47, 59-68, 74-92, 98-144, | C: 5 | 18-66 | 136 | 436 |
| | | 149-158, 173-180, 200-210, 216-223, | | | | |
| | | 239-250 | | | | |
| ECs4762 | putative alpha helix chain | 4-30, 42-52, 59-67, 70-76, 80-86, 138-147, | E: 3 | 375-452 | 137 | 437 |
| | | 154-159, 206-217, 226-232, 240-248, | | | | |
| | | 250-257, 259-265, 280-294, 299-326, | | | | |
| | | 328-334, 340-355, 393-398, 415-422, | | | | |
| | | 440-447, 452-458 | | | | |
| ECs4784 | putative GTP-binding | 4-12, 20-31, 43-49, 100-118, 121-138, | N: 2 | 55-75 | 138 | 438 |
| | protein | 141-148, 153-161, 167-177, 206-213, | | | | |
| | | 225-231, 235-240, 256-267, 277-287, | | | | |
| | | 301-322, 325-333, 336-360, 381-388, | | | | |
| | | 400-415, 447-452, 459-472 | | | | |
| ECs4860 | essential cell division | 4-10, 29-56, 93-99, 119-124, 133-140, | M: 31 | 61-198 | 139 | 439 |
| | protein | 159-171, 187-195, 200-214, 221-232, | | | | |
| | | 249-255, 263-271, 285-291, 310-316 | | | | |
| ECs4901 | pantothenate kinase | 9-15, 48-65, 72-79, 87-102, 104-115, | H: 9 | 1-54 | 140 | 440 |
| | | 118-124, 126-138, 153-185, 188-207, | | | | |
| | | 212-239, 257-265, 297-304, 306-313 | | | | |
| ECs4910 | RNA polymerase beta | 18-38, 48-62, 68-107, 143-158, 167-181 | H: 4, M: 13 | 1056-1199 | 141 | 441 |
| | subunit | 193-198, 205-213, 220-231, 239-245, | | | | |
| | | 258-264, 279-300, 308-314, 318-328, | | | | |
| | | 343-354, 360-367, 425-433, 465-474, | | | | |
| | | 496-505, 508-514, 529-536, 545-562, | | | | |
| | | 572-580, 587-593, 595-609, 612-618, | | | | |
| | | 627-637, 642-649, 652-673, 688-693, | | | | |
| | | 696-701, 707-736, 748-754, 766-776, | | | | |
| | | 779-786, 791-797, 815-825, 830-842, | | | | |
| | | 857-865, 868-876, 880-887, 898-905, | | | | |
| | | 911-923, 925-936, 961-983, 1011-1018, | | | | |
| | | 1043-1059, 1073-1079, 1093-1104, | | | | |
| | | 1110-1116, 1135-1144, 1146-1163, | | | | |
| | | 1183-1189, 1196-1204, 1222-1242, | | | | |
| | | 1250-1262, 1275-1296, 1322-1330 | | | | |
| ECs4914 | thiamin biosynthesis | 15-27, 35-40, 47-55, 57-73, 77-93, 103-112, | C: 2 | 83-115 | 142 | 442 |
| | protein ThiG | 126-138, 141-179, 192-218, 224-237, | | | | |
| | | 244-257, 263-278 | | | | |
| ECs5012 | hypothetical protein | 4-22, 24-29, 36-43, 63-75, 90-96, 118-128, | D: 2, E: 2 | 70-79, 473-516 | 143 | 443 |
| | | 137-145, 168-182, 198-210, 212-221, | | | | |
| | | 242-250, 289-316, 318-323, 327-339, | | | | |
| | | 381-387, 401-411, 424-434, 443-449, | | | | |
| | | 453-465, 485-498, 500-508, 510-515, | | | | |
| | | 521-528, 538-545, 554-560, 574-606, | | | | |
| | | 619-627, 645-658, 681-688 | | | | |
| ECs5026 | regulator for SOS regulon | 8-18, 45-50, 52-62, 76-82, 84-107, 109-116 | L: 4, P: 14 | 9-73 | 144 | 444 |
| | | 130-137, 141-150, 152-158, 164-170, | | | | |
| | | 175-186, 188-196 | | | | |
| ECs5061 | selenopolypeptide subunit | 4-24, 39-46, 84-95, 133-151, 166-174, | C: 2, F: 4 | 40-166, 577-605 | 145 | 445 |
| | of formate dehydrogenase | 179-189, 196-203, 212-218, 223-236, | | | | |
| | | 240-246, 255-261, 266-275, 286-293, | | | | |
| | | 299-316, 323-331, 347-358, 368-380, | | | | |
| | | 382-389, 391-398, 410-417, 420-429, | | | | |
| | | 437-445, 451-456, 464-471, 504-514, | | | | |
| | | 523-532, 539-545, 553-567, 572-588, | | | | |
| | | 594-603, 620-630, 636-641, 656-663, | | | | |
| | | 665-672, 676-687, 693-700 | | | | |
| ECs5079 | ATP-binding component | 4-42, 48-59, 74-90, 92-119, 121-149, | L: 3 | 1-9 | 146 | 446 |
| | of phosphonate | 163-180, 185-192, 199-209 | | | | |
| | transporter | | | | | |
| ECs5087 | periplasmic binding | 5-26, 60-76, 104-114, 119-128, 136-141, | C: 8 | 294-334 | 147 | 447 |
| | protein component of Pn | 156-167, 186-198, 218-237, 260-267, | | | | |
| | transporter | 275-290, 328-335 | | | | |
| ECs5091 | putative vimentin | 4-10, 14-37, 40-47, 68-77, 87-95, 103-111, | O: 1 | 302-448 | 148 | 448 |
| | | 116-153, 170-237, 245-251, 253-274, | | | | |
| | | 280-299, 311-318, 321-338, 364-371, | | | | |
| | | 378-392, 395-430, 438-451, 458-475, | | | | |
| | | 479-507, 520-526, 542-560, 573-586, | | | | |
| | | 591-598, 608-614, 636-668, 678-690, | | | | |
| | | 692-698, 702-717, 724-731 | | | | |
| ECs5147 | delta(2)- | 5-17, 23-48, 60-73, 75-82, 98-108, 110-128, | G: 5, H: 2, N: 4 | 63-147 | 149 | 449 |
| | isopentenylpyrophosphate | 146-160, 168-180, 191-213, 229-237, | | | | |
| | tRNA-adenosine | 240-252, 269-277, 305-313 | | | | |
| | transferase | | | | | |
| ECs5153 | adenylosuccinate | 4-10, 19-35, 41-47, 51-60, 70-80, 91-115, | E: 3 | 356-391 | 150 | 450 |
| | synthetase | 170-191, 194-211, 226-232, 234-241, | | | | |
| | | 256-273, 289-294, 311-349, 358-363, | | | | |
| | | 392-400, 406-416 | | | | |
| ECs5191 | 2:3-cyclic-ucleotide 2- | 5-25, 50-57, 67-75, 78-86, 94-112, 122-145, | H: 6 | 7-127 | 151 | 451 |
| | phosphodiesterase | 152-165, 171-183, 193-199, 217-235, | | | | |
| | | 238-253, 255-264, 281-287, 294-303, | | | | |
| | | 309-314, 319-324, 327-341, 349-355, | | | | |
| | | 364-382, 384-392, 397-411, 419-427, | | | | |
| | | 435-452, 455-463, 488-504, 536-541, | | | | |
| | | 558-563, 568-574, 595-601, 614-620, | | | | |
| | | 637-644 | | | | |
| ECs5218 | repressor of treA, B, C | 10-16, 39-45, 62-91, 102-114, 120-127, | N: 2 | 224-295 | 152 | 452 |
| | | 136-147, 152-159, 163-173, 178-188, | | | | |
| | | 196-217, 223-231, 234-254, 257-267, | | | | |
| | | 270-283, 290-300, 306-312 | | | | |
| ECs5276 | export and assembly outer | 4-9, 12-21, 23-52, 54-65, 74-83, 103-117, | Q: 7 | 283-379 | 153 | 453 |
| | membrane protein | 131-141, 144-163, 171-177, 183-189, | | | | |
| | | 205-212, 252-262, 297-304, 308-314, | | | | |
| | | 320-329, 343-349, 357-370, 375-380, | | | | |
| | | 395-405, 434-441, 456-465, 474-484, | | | | |
| | | 505-514, 528-536, 540-549, 576-582, | | | | |
| | | 597-607, 616-622, 634-641, 648-654, | | | | |
| | | 690-713, 715-724, 743-751, 757-763, | | | | |
| | | 772-789, 791-802, 809-814, 828-837, | | | | |
| | | 840-846, 853-875 | | | | |
| ECs5315 | methyl-accepting | 4-27, 40-46, 55-62, 84-100, 108-114, | P: 4 | 252-272 | 154 | 454 |
| | chemotaxis protein | 118-123, 132-145, 165-171, 178-183, | | | | |
| | | 192-223, 226-232, 234-243, 276-282, | | | | |
| | | 299-305, 326-334, 340-351, 357-371, | | | | |
| | | 374-387, 395-406, 417-437, 443-452, | | | | |
| | | 470-478, 485-494, 496-503, 508-521, | | | | |
| | | 527-537, 541-546 | | | | |
| ECs5350 | soluble lytic murein | 4-36, 45-50, 58-63, 69-80, 89-97, 99-109, | P: 4 | 418-432 | 155 | 455 |
| | transglycosylase | 111-118, 126-132, 141-147, 172-184, | | | | |
| | | 188-197, 208-215, 220-231, 236-241, | | | | |
| | | 253-264, 271-280, 288-297, 342-347, | | | | |
| | | 361-367, 375-382, 388-394, 401-406, | | | | |
| | | 408-414, 441-447, 452-458, 466-476, | | | | |
| | | 483-491, 503-510, 521-528, 539-545, | | | | |
| | | 547-558, 566-576, 584-589, 606-617, | | | | |
| | | 624-636 | | | | |
| b1368 | putative alpha helix | 7-14, 5-32, 6-72, 95-100, 108-114, 123-135, | D: 4 | 149-248 | 156 | 456 |
| | protein | 143-153, 203-221, 224-230, 260-269, | | | | |
| | | 290-297, 302-308, 320-328, 333-339 | | | | |
| b2969 | putative general secretion | 21-27, 30-48, 55-65, 70-90, 97-107, | N: 4 | 240-257 | 157 | 457 |
| | pathway for protein | 122-128, 135-166, 172-180, 184-199, | | | | |
| | export | 205-224, 237-247, 252-269, 278-283 | | | | |
| b2986 | orf, hypothetical protein | 4-14, 20-33, 36-43, 49-60, 72-114, 117-123, | N: 2 | 58-89 | 158 | 458 |
| | | 125-132, 138-143, 157-175, 184-204, | | | | |
| | | 208-217 | | | | |
| b3480 | ATP-binding protein of | 4-15, 23-36, 38-47, 54-64, 92-103, 117-126, | L: 12 | 70-162 | 159 | 459 |
| | nickel transport system | 135-155, 157-200, 202-223, 231-239, | | | | |
| | | 246-261 | | | | |
| b3689 | orf, hypothetical protein | 4-10, 35-45, 56-92, 108-114, 127-146, | L: 26 | 93-188 | 160 | 460 |
| | | 160-186, 194-203, 225-234, 242-251, | | | | |
| | | 272-283, 285-306, 310-315, 322-330, | | | | |
| | | 358-371, 373-382, 389-396 | | | | |
| EAEC11 | 175 aa, hypothetical | 18-32, 35-82, 85-115, 119-142, 149-172 | N: 14 | 4-36 | 161 | 461 |
| | protein | | | | | |
| EAEC12 | Pic serin protease from | 6-14, 20-28, 35-55, 64-87, 100-109, | B: 3 | 414-503 | 162 | 462 |
| | Shigella flexneri 2a: | 144-149, 189-208, 210-218, 221-227, | | | | |
| | SF2973: 100% | 242-247, 254-264, 283-297, 301-308, | | | | |
| | | 310-322, 351-358, 372-378, 383-389, | | | | |
| | | 421-432, 447-460, 537-545, 550-558, | | | | |
| | | 581-593, 595-606, 645-658, 677-688 | | | | |
| EAEC15 | 75 aa, homology to part of | 9-34, 47-63 | N: 3 | 37-51 | 163 | 463 |
| | TraD (40 aa)[E. coli, strain | | | | | |
| | CR63], plasmid F | | | | | |
| EAEC16 | surface exclusion protein | 6-22, 50-58, 73-101, 119-128, 139-154, | F: 5 | 1-126 | 164 | 464 |
| | | 167-173, 209-217, 227-234 | | | | |
| EAEC17 | membrane protein traD - | 10-34, 37-44, 72-78, 87-100, 111-117, | F: 3 | 341-443 | 165 | 465 |
| | Escherichia coli | 122-143, 177-196, 207-229, 232-249, | | | | |
| | | 255-261, 268-278, 289-300, 315-349, | | | | |
| | | 351-358, 371-378, 386-394, 404-466, | | | | |
| | | 468-480 | | | | |
| EAEC18 | Sequence not present in | 16-22, 30-35, 45-52, 54-64, 74-84, 90-98, | D: 17 | 341-484 | 166 | 466 |
| | current EAEC genome, | 120-125, 135-148, 156-162, 166-186, | | | | |
| | but found in E. coli | 190-199, 201-215, 226-232, 262-270, | | | | |
| | plasmid pLG13. Conjugal | 281-289, 322-331, 335-340, 367-372, | | | | |
| | mobilization protein | 379-385, 415-426, 438-445, 455-466, | | | | |
| | MobA (plasmid transfer) | 472-479, 492-497 | | | | |
| | E. coli 75% | | | | | |
| EAEC19 | Sequence not present in | 4-31, 33-66, 77-83, 90-101, 118-135, | Q: 18 | 529-629 | 167 | 467 |
| | current EAEC genome. | 161-166, 168-189, 203-209, 216-222, | | | | |
| | Escherichia coil strain | 231-236, 269-277, 279-290, 298-310, | | | | |
| | H10407 plasmid pCS1 | 341-347, 371-376, 380-387, 389-395, | | | | |
| | secreted autotransporter | 410-419, 446-455, 465-471, 476-484, | | | | |
| | protein EatA (eatA) gene. | 513-522, 532-537, 545-556, 560-585, | | | | |
| | 100%. Homology to | 603-610, 630-657, 660-672, 694-704, | | | | |
| | (EAEC12) SepA Shigella | 717-728, 738-745, 754-766, 782-792, | | | | |
| | | 794-811, 813-819, 833-853, 901-906, | | | | |
| | | 912-917, 951-979, 985-991, 998-1004, | | | | |
| | | 1013-1019, 1052-1065, 1080-1086, | | | | |
| | | 1124-1130, 1142-1150, 1168-1176, | | | | |
| | | 1182-1193, 1209-1218, 1234-1245, | | | | |
| | | 1271-1277, 1284-1298, 1301-1308, | | | | |
| | | 1339-1345 | | | | |
| EAEC20 | Sequence not present in | 21-28, 57-71 | N: 10 | 15-46 | 168 | 468 |
| | current EAEC genome. | | | | | |
| | E. coli plasmid pCoID-157 | | | | | |
| | DNA 97%. 7.6 kd ORF | | | | | |
| EAEC21 | Hypothetical protein | 63-99, 101-109, 111-137, 143-172, 175-200 | G: 6 | 11-48 | 169 | 469 |
| EAEC23 | Hypothetical protein | 18-32, 35-82, 85-115, 119-142, 149-172 | G: 5 | 6-39 | 170 | 470 |
| EAEC24 | Hypothetical protein | 67-74, 88-94, 112-118, 127-138, 155-169, | H: 4 | 26-129 | 171 | 471 |
| | | 171-180, 183-190, 196-205, 243-249, | | | | |
| | | 260-271, 308-344, 346-373, 381-414, | | | | |
| | | 416-457, 473-513, 515-524, 528-535, | | | | |
| | | 539-544, 556-566, 572-580, 585-590 | | | | |
| EAEC25 | Hypothetical protein | 17-40, 47-66, 70-78 | H: 5 | 29-102 | 172 | 472 |
| EAEC27 | Hypothetical protein | 4-9, 23-41, 44-51, 58-64, 70-86, 94-111 | L: 6 | 47-77 | 173 | 473 |
| EAEC29 | Hypothetical protein | 4-22, 29-48, 50-58, 62-69, 71-78 | L: 7 | 6-35 | 174 | 474 |
| EAEC141 | Hypothetical ABC | 8-19, 31-47, 49-58, 64-79, 84-96 | L: 83 | 7-52 | 175 | 475 |
| | transporter | | | | | |
| EAEC147 | 139 aa, hypothetical | 4-17, 19-26, 41-49, 63-87, 92-99, 113-131 | D: 43 | 9-118 | 176 | 476 |
| | protein | | | | | |
| EAEC160 | dipeptide transport | 10-37, 55-68, 71-78, 92-98, 115-122, | A: 2 | 186-224 | 177 | 477 |
| | protein | 131-138, 149-158, 163-170, 172-189, | | | | |
| | | 212-219, 239-257, 259-271, 289-302, | | | | |
| | | 304-320, 322-340, 359-366, 373-384, | | | | |
| | | 400-412, 444-453, 460-474, 485-527 | | | | |
| ETLAB27 | No homology | 6-13 | E: 4 | Clone only | 178 | 478 |
| ETLAG82 | No homology | None | E: 6 | Clone only | 179 | 479 |
| ETLAD13 | No homology | 5-12 | E: 5 | Clone only | 180 | 480 |
| ETLAD81 | No homology | 6-14 | E: 3 | Clone only | 181 | 481 |
| ETLAP25 | No homology | None | P: 4 | Clone only | 182 | 482 |
| ETLAK26 | No homology | 4-10 | P: 3 | Clone only | 183 | 483 |
| ETLAT10 | No homology | None | P: 2 | Clone only | 184 | 484 |
| ETLAX86 | No homology | None | N: 6 | Clone only | 185 | 485 |
| ETLAY83 | No homology | 4-19 | N: 3 | Clone only | 186 | 486 |
| ETLBA51 | No homology | 9-14 | N: 2 | Clone only | 187 | 487 |
| ARF0254 | Hypothetical protein | 4-44, 47-60, 65-72, 92-98 | C: 2 | 61-95 | 188 | 488 |
| ARF0386 | Hypothetical protein | None | E: 5 | 2-17 | 189 | 489 |
| ARF0600 | Hypothetical protein | 19-29, 39-45, 52-59 | C: 10 | 7-32 | 190 | 490 |
| ARF0729 | Hypothetical protein | 6-20, 36-44 | N: 1 | 78-97 | 191 | 491 |
| ARF0842 | Hypothetical protein | 5-12 | N: 3 | 1-22 | 192 | 492 |
| ARF0965 | Hypothetical protein | 17-24, 26-41, 50-55 | C: 2 | 8-32 | 193 | 493 |
| ARF0968 | Hypothetical protein | 7-18 | E: 13 | 13-22 | 194 | 494 |
| ARF1004 | Hypothetical protein | 24-32 | C: 6 | 3-28 | 195 | 495 |
| ARF1396 | Hypothetical protein | 6-11, 14-35, 40-56 | C: 4 | 17-35 | 196 | 496 |
| ARF1480 | Hypothetical protein | 4-12, 17-23, 42-53, 69-81 | E: 10 | 45-64 | 197 | 497 |
| ARF1603 | Hypothetical protein | 4-25, 28-34, 37-43, 59-69, 104-114 | C: 2, G: 4, N: 5 | 52-117 | 198 | 498 |
| ARF1806 | Hypothetical protein | 4-12 | N: 2 | 4-22 | 199 | 499 |
| ARF2582 | Hypothetical protein | 4-9 | E: 5 | 1-19 | 200 | 500 |
| ARF2816 | Hypothetical protein | 4-22 | E: 37, P: 2 | 2-19 | 201 | 501 |
| ARF2885 | Hypothetical protein | 13-36, 48-63, 80-101, 141-149, 165-176, | A: 3 | 28-54 | 202 | 502 |
| | | 184-198 | | | | |
| ARF3066 | Hypothetical protein | 4-14, 21-26 | P: 6 | 21-29 | 203 | 503 |
| ARF3107 | Hypothetical protein | 4-26 | N: 2 | 20-36 | 204 | 504 |
| ARF3116 | Hypothetical protein | 4-13 | P: 2 | 7-22 | 205 | 505 |
| ARF3364 | Hypothetical protein | 7-13, 21-34 | D: 2 | 26-54 | 206 | 506 |
| ARF3558 | Hypothetical protein | 4-21, 25-44, 59-68 | B: 5 | 1-92 | 207 | 507 |
| ARF3629 | Hypothetical protein | 4-14, 26-33 | G: 3 | 12-33 | 208 | 508 |
| ARF3673 | Hypothetical protein | 12-31, 46-60, 87-94 | B: 3 | 1-98 | 209 | 509 |
| ARF3699 | Hypothetical protein | 5-14 | N: 3 | 4-22 | 210 | 510 |
| ARF3942 | Hypothetical protein | 6-15, 34-52 | E: 2 | 14-36 | 211 | 511 |
| ARF4105 | Hypothetical protein | 5-18, 27-33, 40-48 | C: 2 | 17-39 | 212 | 512 |
| ARF4206 | Hypothetical protein | 8-17, 63-70, 89-100 | A: 3, C: 12, | 21-52 | 213 | 513 |
| | | | L: 11 | | | |
| ARF4334 | Hypothetical protein | 4-18, 21-31, 51-68 | A: 5 | 34-66 | 214 | 514 |
| ARF4425 | Hypothetical protein | 4-9, 14-19 | L: 6 | 3-31 | 215 | 515 |
| ARF4467 | Hypothetical protein | 4-10, 21-30, 37-51 | L: 8 | 11-44 | 216 | 516 |
| ARF4491 | Hypothetical protein | 4-9, 32-38 | P: 5 | 7-22 | 217 | 517 |
| ARF4639 | Hypothetical protein | 4-22, 29-36, 38-47 | C: 4 | 12-43 | 218 | 518 |
| ARF4661 | Hypothetical protein | 4-12, 14-23 | N: 3 | 26-37 | 219 | 519 |
| ARF4692 | Hypothetical protein | 4-11, 20-46 | A: 1 | 32-59 | 220 | 520 |
| ARF4739 | Hypothetical protein | 4-10 | A: 6 | 1-30 | 221 | 521 |
| ARF4851 | Hypothetical protein | 4-28, 32-50 | A: 5 | 17-51 | 222 | 522 |
| ARF4921 | Hypothetical protein | 14-28 | N: 1 | 2-14 | 223 | 523 |
| ARF5002 | Hypothetical protein | 4-25 | A: 2 | 12-40 | 224 | 524 |
| ARF5020 | Hypothetical protein | 33-41, 43-53 | C: 2 | 16-39 | 225 | 525 |
| ARF5159 | Hypothetical protein | 9-16 | E: 1 | 7-18 | 226 | 526 |
| ARF5319 | Hypothetical protein | 4-11, 24-31 | E: 2 | 18-34 | 227 | 527 |
| ARF5347 | Hypothetical protein | 14-20, 48-56, 62-69, 81-87 | G: 23 | 4-56 | 228 | 528 |
| ARF-b1398 | Hypothetical protein | 8-16 | N: 2 | 13-39 | 229 | 529 |
| ARF-b3374 | Hypothetical protein | 11-16, 26-40, 50-63 | G: 2 | 6-48 | 230 | 530 |
| ARF-b3577 | Hypothetical protein | 23-34, 59-72 | G: 2 | 1-28 | 231 | 531 |
| ARFF11 | Hypothetical protein | 11-16, 26-82, 95-101, 103-113, 120-164, | A: 2 | 46-77 | 232 | 532 |
| (ARF4418) | | 179-185, 187-194, 224-248, 255-263, | | | | |
| | | 276-293, 296-301, 304-312, 314-320, | | | | |
| | | 351-374, 390-398, 401-407, 420-426, | | | | |
| | | 434-444, 454-475, 481-508, 521-531, | | | | |
| | | 535-549 | | | | |
| ARF20 | Hypothetical protein | 6-30, 32-53, 69-76, 78-86, 96-112, 121-135, | G: 3 | 6-30 | 233 | 533 |
| (ARF5062) | | 142-175, 177-199, 201-255, 263-269, | | | | |
| | | 277-288, 290-303, 307-345, 353-364, | | | | |
| | | 388-403, 443-455, 462-474, 480-485 | | | | |
| ARF21 | Hypothetical protein | 12-61 | A: 7 | 14-46 | 234 | 534 |
| ARF22 | Hypothetical protein | 4-12, 24-33, 39-51, 57-63, 78-87 | C: 3 | 26-51 | 235 | 535 |
| ARF23 | Hypothetical protein | 8-15, 29-40, 48-54 | C: 3 | 33-56 | 236 | 536 |
| ARF132 | Hypothetical protein | 22-31, 59-69 | C: 2 | 70-100 | 237 | 537 |
| (ARF4862) | | | | | | |
| ARF152 | Hypothetical protein | 12-61 | A: 6 | 12-43 | 238 | 538 |
| CRF0147 | Hypothetical protein | 4-16, 28-39, 62-71, 85-97 | L: 12 | 54-85 | 239 | 539 |
| CRF0173 | Hypothetical protein | 10-20, 23-31, 35-42, 48-62 | G: 4 | 30-53 | 240 | 540 |
| CRF0372 | Hypothetical protein | 9-41, 46-52, 70-85 | A: 5 | 63-89 | 241 | 541 |
| CRF0451 | Hypothetical protein | 18-34, 36-46, 71-83, 95-101, 130-143, | P: 7 | 96-115 | 242 | 542 |
| | | 149-161 | | | | |
| CRF0464 | Hypothetical protein | 4-18, 26-66, 68-95, 100-110, 120-135, | P: 5 | 160-176 | 243 | 543 |
| | | 143-165, 168-175, 177-198 | | | | |
| CRF0575 | Hypothetical protein | 4-16, 21-34, 51-56 | P: 2 | 10-29 | 244 | 544 |
| CRF0600 | Hypothetical protein | 4-23, 29-37, 40-70, 78-91, 98-111 | N: 3 | 89-115 | 245 | 545 |
| CRF1135 | Hypothetical protein | 4-34, 56-62 | P: 5 | 36-54 | 246 | 546 |
| CRF1408 | Hypothetical protein | 4-9 | G: 4 | 1-29 | 247 | 547 |
| CRF1476 | Hypothetical protein | 10-22, 24-37, 52-57, 74-81 | G: 5 | 16-69 | 248 | 548 |
| CRF1480 | Hypothetical protein | 4-16, 42-70, 78-93, 95-101, 103-111 | E: 2 | 64-88 | 249 | 549 |
| CRF1640 | Hypothetical protein | 4-9, 22-48, 51-59, 64-94, 100-124, 130-135 | N: 4 | 123-134 | 250 | 550 |
| CRF1683 | Hypothetical protein | 10-20, 27-40, 48-57 | C: 20, E: 75, | 15-58 | 251 | 551 |
| | | | G: 42 | | | |
| CRF1902 | Hypothetical protein | 24-37 | C: 1 | 12-30 | 252 | 552 |
| CRF2012 | Hypothetical protein | 30-42, 51-56, 67-76, 79-96 | C: 11 | 22-61 | 253 | 553 |
| CRF2015 | Hypothetical protein | 11-22, 28-35 | P: 9 | 18-33 | 254 | 554 |
| CRF2072 | Hypothetical protein | 4-9, 13-21, 37-42 | E: 14 | 23-36 | 255 | 555 |
| CRF2329 | Hypothetical protein | 4-12 | E: 4 | 12-22 | 256 | 556 |
| CRF2336 | Hypothetical protein | 39-50, 71-89, 95-106, 126-139, 154-162 | Q: 6 | 58-143 | 257 | 557 |
| CRF2570 | Hypothetical protein | 5-26, 38-46, 54-62, 69-81, 87-99, 103-117, | L: 16 | 136-167 | 258 | 558 |
| | | 120-136, 138-161, 168-189, 201-207 | | | | |
| CRF2633 | Hypothetical protein | 4-21, 33-39, 41-55, 61-68, 73-98, 104-110, | P: 2 | 97-115 | 259 | 559 |
| | | 121-127, 131-156 | | | | |
| CRF2645 | Hypothetical protein | 9-26, 36-48 | N: 10 | 21-40 | 260 | 560 |
| CRF2649 | Hypothetical protein | 4-12, 26-32 | E: 2 | 12-24 | 261 | 561 |
| CRF2696 | Hypothetical protein | 4-12, 17-23, 39-62 | A: 5 | 8-34 | 262 | 562 |
| CRF2937 | Hypothetical protein | 17-41, 47-66 | L: 19 | 12-30 | 263 | 563 |
| CRF3041 | Hypothetical protein | 4-11, 15-25, 33-52 | G: 6 | 1-27 | 264 | 564 |
| CRF3088 | Hypothetical protein | 4-11, 33-40, 48-76, 96-104 | E: 10 | 84-106 | 265 | 565 |
| CRF3100 | Hypothetical protein | 15-22 | E: 2 | 11-26 | 266 | 566 |
| CRF3160 | Hypothetical protein | 8-19, 44-53, 61-71, 78-85, 97-107 | C: 4 | 49-89 | 267 | 567 |
| CRF3314 | Hypothetical protein | 13-23, 31-44, 59-66, 84-90, 96-110 | B: 3 | 47-147 | 268 | 568 |
| CRF3625 | Hypothetical protein | 4-24, 56-73, 83-97, 112-132, 140-150, | G: 7 | 111-146 | 269 | 569 |
| | | 161-184 | | | | |
| CRF3797 | Hypothetical protein | 4-17, 19-26, 41-48, 63-87, 92-99, 113-131 | M: 11 | 10-136 | 270 | 570 |
| CRF3808 | Hypothetical protein | 4-11, 17-26 | C: 5 | 6-32 | 271 | 571 |
| CRF3936 | Hypothetical protein | 26-38, 48-55, 72-86, 90-107, 123-132, | N: 6 | 3-19 | 272 | 572 |
| | | 134-161, 181-187 | | | | |
| CRF4119 | Hypothetical protein | 4-19, 26-35 | C: 32 | 11-46 | 273 | 573 |
| CRF4266 | Hypothetical protein | 5-10, 21-38, 42-70, 84-103 | C: 6 | 92-127 | 274 | 574 |
| CRF4317 | Hypothetical protein | 6-17, 34-40, 42-51, 75-85, 94-100 | P: 5 | 38-63 | 275 | 575 |
| CRF4320 | Hypothetical protein | 6-13, 21-27, 29-35, 57-64 | C: 6 | 32-63 | 276 | 576 |
| CRF4336 | Hypothetical protein | 4-19, 22-48, 54-62, 73-91, 94-109 | P: 3 | 75-98 | 277 | 577 |
| CRF4344 | Hypothetical protein | 9-25 | N: 3 | 12-28 | 278 | 578 |
| CRF4364 | Hypothetical protein | 16-21 | P: 12 | 8-25 | 279 | 579 |
| CRF4393 | Hypothetical protein | 4-24, 26-32, 45-55, 58-67, 76-93, 104-111, | Q: 3 | 33-119 | 280 | 580 |
| | | 117-122, 128-136 | | | | |
| CRF4397 | Hypothetical protein | 5-11, 31-37, 56-63, 66-84 | P: 5 | 32-58 | 281 | 581 |
| CRF4483 | Hypothetical protein | 6-69, 75-87, 89-111, 149-156 | H: 4 | 52-139 | 282 | 582 |
| CRF4752 | Hypothetical protein | 4-13, 19-29, 49-68 | G: 12 | 24-49 | 283 | 583 |
| CRF4786 | Hypothetical protein | 19-25, 27-33, 43-84, 86-92, 111-118, | C: 6 | 95-124 | 284 | 584 |
| | | 125-136, 138-147 | | | | |
| CRF4885 | Hypothetical protein | 20-29, 50-56, 63-85, 89-98, 110-128 | G: 14 | 2-29 | 285 | 585 |
| CRF5063 | Hypothetical protein | 4-11, 41-47, 62-71 | G: 3 | 7-34 | 286 | 586 |
| CRF5138 | Hypothetical protein | 23-30, 48-57, 67-72, 81-89 | N: 5 | 11-31 | 287 | 587 |
| CRF5282 | Hypothetical protein | 14-27, 50-62 | A: 4 | 22-57 | 288 | 588 |
| CRF-b0497 | Hypothetical protein | 4-17, 23-37 | P: 10 | 13-33 | 289 | 589 |
| CRF-b1399 | Hypothetical protein | 5-33, 38-57, 60-68 | N: 10 | 18-37 | 290 | 590 |
| CRF-b2972 | Hypothetical protein | 4-19, 44-51 | G: 5 | 24-45 | 291 | 591 |
| CRF-b4332 | Hypothetical protein | 17-40, 47-66, 70-78 | G: 3 | 67-101 | 292 | 592 |
| CRF13 | Hypothetical protein | 4-20, 24-29, 43-55, 57-63, 74-83, 137-157, | H: 8 | 61-120 | 293 | 593 |
| | | 171-179, 181-192, 237-243, 273-279, | | | | |
| | | 300-310, 312-321, 324-330, 366-380, | | | | |
| | | 401-417 | | | | |
| CRF28 | Hypothetical protein | 6-21 | P: 30 | 17-43 | 294 | 594 |
| CRF29 | Hypothetical protein | 11-17 | N: 1 | 9-31 | 295 | 595 |
| CRF30 | Hypothetical protein | 18-28, 38-44, 53-59, 64-74, 78-85 | B: 3 | 10-96 | 296 | 596 |
| CRF31 | Hypothetical protein | 12-40, 49-58 | C: 23 | 5-49 | 297 | 597 |
| CRF32 | Hypothetical protein | 28-34, 46-52, 54-68, 72-85, 95-104 | C: 3 | 66-94 | 298 | 598 |
| CRF33 | Hypothetical protein | 4-14 | C: 3 | 1-25 | 299 | 599 |
| CRF34 | Hypothetical protein | 4-18, 30-37, 51-65, 83-89, 99-105, 108-136 | D: 3 | 24-98 | 300 | 600 |

TABLE 2
Immunogenic proteins identified from S. flexneri 2a by bacterial surface display.

| | | | No. of | Location | | |
|---|---|---|---|---|---|---|
| | | | selected | of | | |
| | | | clones | identified | | Seq. |
| S. flexneri | Putative | | per ORF | immunogenic | Seq. | ID |
| antigenic | function | | and | region | ID | (Pro- |
| protein | (by homology) | predicted immunogenic aa* | screen | (aa) | (DNA) | tein) |
| icsB | intercellular | 26-34, 36-44, 68-78, 85-92, 96-101, 127-134, 141-148, | D: 4 | 147-240 | 925 | 997 |
| | spread protein | 156-166, 186-192, 244-256, 281-287, 291-301, 308-316, | | | | |
| | | 321-343, 368-382, 385-391, 394-404, 414-429, | | | | |
| | | 453-465, 471-489 | | | | |
| ipaA | Invasion | 10-18, 26-48, 58-68, 72-78, 94-105, 115-130, 155-164, | A: 5, | 1-123, 161-546 | 926 | 998 |
| | plasmid | 170-177, 179-185, 201-219, 243-260, 267-277, 295-302, | B: 74, | | | |
| | antigen A | 350-376, 398-403, 429-437, 451-462, 471-478, | C: 8, D: 81 | | | |
| | | 504-512, 563-568, 570-583, 589-594, 614-629 | | | | |
| ipaB | Invasion | 10-20, 35-45, 59-93, 163-168, 190-196, 200-210, 233-261, | B: 22, | 100-301, | 927 | 999 |
| | plasmid | 270-279, 306-329, 331-353, 363-381, 388-394, | D: 17 | 446-518 | | |
| | antigen B | 399-425, 443-460, 462-474, 477-498, 506-516, 522-542, | | | | |
| | | 546-559, 570-577 | | | | |
| ipaC | Invasion | 4-13, 28-34, 52-71, 78-90, 118-140, 147-156, 167-187, | B: 155, | 31-117, | 928 | 1000 |
| | plasmid | 264-275, 286-292, 305-310, 328-334, 340-346, 351-362 | C: 6, | 165-354 | | |
| | antigen C | | D: 129 | | | |
| ipaD | Invasion | 38-45, 76-84, 91-103, 111-118, 147-162, 166-177, 187-201, | B: 3, D: 18 | 18-100, | 929 | 1001 |
| | plasmid | 208-215, 242-249, 267-274, 295-301, 309-322 | | 111-296 | | |
| | antigen D | | | | | |
| ipaH14 | invasion | 4-9, 25-39, 41-47, 70-78, 82-103, 206-140, 152-188, | A: 6, | 199-383, | 930 | 1002 |
| | plasmid | 192-198, 200-207, 211-217, 232-251, 271-277, 285-299, | B: 13, | 454-579 | | |
| | antigen H14 | 307-314, 323-335, 344-370, 376-382, 388-398, | C: 30, D: 9 | | | |
| | | 417-422, 428-446, 448-456, 462-468, 494-504, 526-533, | | | | |
| | | 565-573 | | | | |
| ipaH45 | invasion | 10-27, 50-87, 94-109, 115-128, 134-152, 155-169, 175-193, | B: 4, C: 1, | 206-363 | 931 | 1003 |
| | plasmid | 196-213, 216-233, 235-251, 259-270, 272-283, | D: 1 | | | |
| | antigen H45 | 293-303, 311-318, 327-339, 348-374, 380-386, 392-402, | | | | |
| | | 421-426, 432-450, 452-460, 466-472, 498-508, | | | | |
| | | 530-537 | | | | |
| ipaH78 | invasion | 9-16, 25-31, 48-86, 90-101, 109-127, 131-147, 150-187, | A: 3, B: 3, | 1-118, 208-283, | 932 | 1004 |
| | plasmid | 189-195, 204-211, 213-226, 237-248, 250-261, | D: 9 | 494-564 | | |
| | antigen H78 | 271-281, 289-296, 305-317, 326-352, 358-364, 370-380, | | | | |
| | | 399-404, 410-428, 430-438, 444-450, 476-486, | | | | |
| | | 508-515, 547-555 | | | | |
| ipaH98 | invasion | 31-37, 57-66, 73-97, 99-117, 119-137, 141-149, 157-172, | A: 7, | 1-98, 233-351, | 933 | 1005 |
| | plasmid | 179-185, 190-196, 203-212, 216-224, 226-238, | B: 16, | 423-538 | | |
| | antigen H98 | 240-251, 261-271, 279-286, 295-307, 316-342, 348-354, | C: 10, | | | |
| | | 360-370, 389-394, 400-418, 420-428, 434-440, | D: 11 | | | |
| | | 466-476, 498-505, 537-545 | | | | |
| mxiH | Type III | 27-37, 44-58, 66-80 | D: 5 | 5-70 | 934 | 1006 |
| | secretion | | | | | |
| | protein | | | | | |
| sepA | secreted | 4-32, 35-69, 92-98, 113-137, 168-190, 224-235, 269-277, | B: 5, D: 10 | 22-86, 138-260, | 935 | 1007 |
| | protease | 279-289, 322-329, 370-376, 380-387, 389-395, | | 490-641 | | |
| | | 411-420, 442-457, 463-473, 476-487, 510-521, 531-554, | | | | |
| | | 561-567, 570-585, 604-610, 612-624, 632-651, | | | | |
| | | 660-672, 690-702, 718-730, 738-745, 782-809, 813-820, | | | | |
| | | 823-829, 844-854, 901-906, 911-917, 959-967, | | | | |
| | | 985-991, 997-1006, 1010-1019, 1036-1045, 1053-1059, | | | | |
| | | 1079-1086, 1124-1132, 1137-1150, 1168-1180, 1182-1193, | | | | |
| | | 1209-1214, 1216-1226, 1231-1245, 1271-1277, | | | | |
| | | 1290-1298, 1301-1307, 1339-1345 | | | | |
| SF0022 | isoleucine | 37-43, 50-57, 65-82, 86-109, 123-129, 141-150, 152-157, | C: 2 | 214-233 | 936 | 1008 |
| | tRNA | 166-172, 179-203, 209-241, 249-296, 298-307, | | | | |
| | synthetase | 312-326, 329-335, 341-348, 364-377, 379-399, 401-409, | | | | |
| | | 411-417, 420-425, 438-444, 461-466, 473-480, | | | | |
| | | 497-505, 522-534, 541-550, 586-597, 608-614, 622-632, | | | | |
| | | 660-666, 679-694, 697-706, 708-731, 737-772, | | | | |
| | | 784-789, 810-825, 837-873, 882-895, 901-928 | | | | |
| SF0132 | orf, partial | 10-16, 18-27, 41-61, 81-90, 133-142 | D: 1 | 43-118 | 937 | 1009 |
| | conserved | | | | | |
| | hypothetical | | | | | |
| | protein | | | | | |
| SF0144 | hydroxamate- | 21-47, 50-69, 83-89, 113-120, 129-146, 152-158, 160-189, | A: 2 | 107-140 | 938 | 1010 |
| | dependent iron | 204-225, 235-249, 251-263, 265-275 | | | | |
| | uptake, | | | | | |
| | cytoplasmic | | | | | |
| | membrane | | | | | |
| SF0267 | Rhs-family | 7-12, 16-24, 28-46, 61-68, 79-85, 87-93, 95-102, 108-123, | C: 13 | 584-613 | 939 | 1011 |
| | protein | 148-164, 166-172, 177-202, 205-215, 231-246, | | | | |
| | | 254-261, 267-273, 280-294, 309-315, 322-329, 337-342, | | | | |
| | | 345-351, 386-394, 406-413, 449-455, 473-480, | | | | |
| | | 490-497, 501-508, 532-539, 576-583, 623-629, 657-665, | | | | |
| | | 681-708, 715-722, 751-757 | | | | |
| SF0722 | 65.4 KD antigen | 10-26, 52-88, 94-109, 115-172, 175-189, 196-210, 215-232, | A: 1, B: 1 | 258-365 | 940 | 1012 |
| | | 244-252, 260-270, 272-283, 293-303, 311-318, | | | | |
| | | 327-339, 348-374, 380-386, 392-402, 421-426, 432-450, | | | | |
| | | 452-460, 466-472, 498-508, 530-537, 569-577 | | | | |
| SF0744 | putative ATP- | 25-46, 59-65, 68-75, 83-90, 93-100, 107-115, 124-135, | A: 5 | 245-271 | 941 | 1013 |
| | binding | 151-177, 183-189, 194-206, 209-215, 219-224, 251-263, | | | | |
| | component of a | 267-276, 305-311, 318-327, 332-338, 350-356, | | | | |
| | transport | 380-396, 406-412, 414-423, 431-437, 453-461, 463-481, | | | | |
| | system | 483-491, 505-510, 513-523, 528-545, 568-575 | | | | |
| SF0765 | outer | 4-19, 21-29, 61-68, 87-95, 103-113, 145-154, 157-170 | A: 1 | 10-76 | 942 | 1014 |
| | membrane | | | | | |
| | protein X | | | | | |
| SF0817 | arginine 3rd | 4-18, 28-36, 41-65, 69-84, 96-103, 106-115, 118-124, | B: 3 | 1-63 | 943 | 1015 |
| | transport | 126-132, 148-156, 169-181, 187-194, 221-227 | | | | |
| | system | | | | | |
| | periplasmic | | | | | |
| | binding protein | | | | | |
| SF0986 | orf, conserved | 5-33, 67-78, 122-129, 141-150, 172-185, 201-209, 217-223, | A: 2 | 309-334 | 944 | 1016 |
| | hypothetical | 235-252, 303-316, 355-368, 383-389, 400-406, | | | | |
| | protein | 411-420, 426-437, 445-451, 459-467, 479-496, 512-517, | | | | |
| | | 523-530, 535-562, 577-584, 590-605, 610-632, | | | | |
| | | 644-654, 663-669, 680-686 | | | | |
| SF1202 | orf, conserved | 31-38, 73-84, 97-121, 124-135, 141-146, 156-170, 174-191, | B: 2 | 85-153 | 945 | 1017 |
| | hypothetical | 205-210 | | | | |
| | protein | | | | | |
| SF1383 | invasion | 24-30, 49-69, 71-111, 113-127, 133-145, 153-169, 172-203, | A: 2, B: 1, | 243-286 | 946 | 1018 |
| | plasmid | 220-236, 247-254, 256-267, 277-287, 295-302, | C: 1 | | | |
| | antigen | 311-323, 332-345, 348-358, 364-370, 376-386, 405-410, | | | | |
| | (ipaH14) | 416-434, 436-444, 450-456, 482-492, 514-521, | | | | |
| | | 548-561 | | | | |
| SF1462 | NADP-specific | 4-10, 22-31, 39-53, 85-94, 102-110, 130-137, 143-149, | C: 38 | 260-345 | 947 | 1019 |
| | glutamate | 154-161, 174-179, 184-190, 197-203, 206-212, 220-227, | | | | |
| | dehydrogenase | 240-248, 251-265, 267-286, 297-310, 338-357, | | | | |
| | | 365-370, 373-382 | | | | |
| SF1841 | putative | 9-48, 54-71, 84-92, 114-145, 160-188, 190-200, 216-236, | D: 12 | 1-94 | 948 | 1020 |
| | transport | 243-255, 261-268, 281-304, 315-322, 330-336, | | | | |
| | periplasmic | 342-351, 370-380, 383-393, 400-408, 412-420, 436-442, | | | | |
| | protein | 452-459, 473-480, 501-512, 518-525, 532-541 | | | | |
| SF1880 | IpaH9.8 | 50-59, 66-81, 90-126, 129-185, 187-205, 213-219, 228-240, | B: 10, D: 2 | 275-374 | 949 | 1021 |
| | | 254-266, 268-279, 289-299, 307-314, 323-335, | | | | |
| | | 344-370, 376-382, 388-398, 417-422, 428-446, 448-456, | | | | |
| | | 462-468, 494-504, 526-533, 565-573 | | | | |
| SF1889 | putative tail | 5-14, 61-74, 91-99, 110-116, 119-136, 138-149, 159-169, | B: 4, D: 3 | 443-534 | 950 | 1022 |
| | length tape | 188-194, 205-227, 236-244, 249-256, 294-305, | | | | |
| | measure | 321-342, 350-364, 373-378, 385-392, 404-420, 422-430, | | | | |
| | protein | 462-470, 491-500, 513-520, 583-591, 617-638, | | | | |
| | precursor | 652-663, 674-680, 684-698, 709-718, 745-753, 757-763, | | | | |
| | | 781-788, 835-841, 844-854, 861-869, 882-890, | | | | |
| | | 898-916, 934-940, 946-952, 979-990 | | | | |
| SF1966 | flagellin | 23-35, 71-77, 94-100, 134-140, 157-163, 188-212, 214-221, | D: 3 | 57-155, | 951 | 1023 |
| | | 262-272, 287-293, 323-331, 360-372, 374-380, | | 324-404 | | |
| | | 402-411, 421-429, 438-443, 462-473, 477-486, 523-528, | | | | |
| | | 530-547 | | | | |
| SF2290 | probable nitrate | 11-36, 43-59, 62-70, 78-90, 159-165, 176-188, 197-202, | C: 2 | 57-82, 445-461 | 952 | 1024 |
| | reductase | 206-213, 220-225, 234-251, 258-275, 286-293, | | | | |
| | | 300-311, 329-337, 352-361, 363-369, 383-396, 399-417, | | | | |
| | | 420-432, 451-466, 472-478, 504-526, 546-552, | | | | |
| | | 558-566, 576-583, 591-609, 621-628, 642-650, 657-663, | | | | |
| | | 676-681, 692-706, 713-724, 734-753, 763-778, | | | | |
| | | 787-807, 819-825 | | | | |
| SF2332 | putative | 14-26, 38-46, 50-57, 76-87, 89-104, 107-112, 123-134, | C: 2 | 133-157 | 953 | 1025 |
| | enzyme | 136-142, 148-162, 173-194, 200-206, 208-216, 226-233, | | | | |
| | | 243-256, 264-307, 338-343, 351-359, 366-376 | | | | |
| SF2423 | orf, conserved | 13-19, 30-37, 46-60, 75-82, 100-105, 109-115, 134-140, | D: 2 | 251-352 | 954 | 1026 |
| | hypothetical | 146-161, 186-192, 199-205, 207-215, 223-230, | | | | |
| | protein | 254-260, 281-289, 297-311, 344-352 | | | | |
| SF2610 | invasion | 5-16, 19-24, 43-54, 56-89, 104-122, 126-139, 144-157, | A: 1, B: 7, | 284-400, | 955 | 1027 |
| | plasmid | 165-202, 232-244, 252-265, 272-277, 281-287, 289-300, | C: 14, D: 5 | 486-600 | | |
| | antigen | 308-320, 344-355, 364-390, 396-402, 408-418, | | | | |
| | | 437-442, 448-466, 468-476, 482-488, 514-524, 546-553, | | | | |
| | | 585-593 | | | | |
| SF2797 | (p)ppGpp | 21-27, 29-46, 50-58, 60-70, 76-98, 100-110, 116-122, | C: 2 | 586-630 | 956 | 1028 |
| | synthetase I | 124-130, 145-166, 170-188, 199-209, 214-221, 229-236, | | | | |
| | (GTP | 244-259, 270-305, 308-314, 319-329, 348-355, | | | | |
| | pyrophosphokinase) | 376-383, 396-442, 446-456, 461-466, 479-485, 513-524, | | | | |
| | | 528-533, 539-546, 556-563, 574-585, 595-602, | | | | |
| | | 604-616, 618-625, 630-647, 649-656, 662-674, 680-686, | | | | |
| | | 689-700, 716-739 | | | | |
| SF2942 | putative | 4-19, 31-49, 82-88, 92-98, 111-141, 146-153, 161-172, | C: 2 | 223-248 | 957 | 1029 |
| | protein | 174-197, 199-214, 218-226, 233-239, 242-250, 256-266, | | | | |
| | transport | 279-293, 298-309, 321-331, 338-345 | | | | |
| | protein | | | | | |
| SF2953 | putative alpha | 24-41, 54-61, 63-77, 83-91, 100-111, 116-121, 128-133, | D: 6 | 144-277 | 958 | 1030 |
| | helix chain | 139-146, 153-164, 166-176, 212-242, 255-266, | | | | |
| | | 269-275, 277-285, 290-299 | | | | |
| SF2968 | exported serine | 4-29, 33-47, 93-109, 117-125, 152-168, 175-180, 213-219, | D: 2 | 549-657 | 959 | 1031 |
| | protease SigA | 224-234, 261-267, 270-284, 321-326, 328-342, | | | | |
| | | 359-365, 383-389, 401-407, 417-424, 471-478, 491-497, | | | | |
| | | 532-537, 545-555, 569-583, 646-652, 688-694, | | | | |
| | | 711-718, 735-745, 794-803, 813-823, 834-839, 851-857, | | | | |
| | | 860-867, 874-882, 895-900, 902-910, 917-923, | | | | |
| | | 933-940, 948-955, 960-966, 1001-1007, 1045-1053, | | | | |
| | | 1058-1066, 1087-1101, 1103-1114, 1130-1139, 1141-1149, | | | | |
| | | 1155-1166, 1192-1198, 1211-1219 | | | | |
| SF2972 | similar to a 523 | 13-48, 57-63, 73-81, 89-103, 107-114, 119-125, 130-140, | C: 1 | 10-23 | 960 | 1032 |
| | aa protein from | 146-167, 175-186, 202-210, 212-220, 233-239, | | | | |
| | Escherichia coli | 255-270, 272-286, 288-301 | | | | |
| SF3061 | suppressor of | 5-41, 48-56, 75-81, 86-93, 100-111, 126-134, 150-156, | C: 11 | 105-135 | 961 | 1033 |
| | ftsI | 168-188, 209-236, 250-257, 260-274, 291-305, 311-335, | | | | |
| | | 337-344, 373-379, 393-406, 423-450, 461-467 | | | | |
| SF3094 | adenylylating | 8-16, 37-88, 105-117, 141-169, 172-178, 189-197, 210-217, | C: 5 | 444-479 | 962 | 1034 |
| | enzyme for | 243-249, 253-260, 269-282, 293-299, 315-332, | | | | |
| | glutamine | 363-373, 378-401, 406-415, 417-423, 454-460, 465-471, | | | | |
| | synthetase | 503-510, 515-521, 538-576, 578-616, 619-625, | | | | |
| | | 636-643, 651-669, 671-686, 691-698, 719-726, 734-748, | | | | |
| | | 762-778, 782-795, 799-810, 821-845, 894-908, | | | | |
| | | 917-925, 937-943, 946-967, 969-975 | | | | |
| SF3482 | cell division | 31-45, 47-55, 68-82, 87-100, 103-108, 112-117, 134-140, | B: 2 | 1-84 | 963 | 1035 |
| | membrane | 157-163, 166-175, 178-185, 197-206, 213-219, | | | | |
| | protein | 234-244, 265-272, 280-289, 292-305, 312-317, 322-328, | | | | |
| | | 335-344, 349-356, 361-372, 376-383, 399-421, | | | | |
| | | 426-444, 455-475, 490-495 | | | | |
| SF3774 | orf, conserved | 4-10, 43-53, 64-100, 116-123, 135-141, 145-154, 164-194, | D: 2 | 70-188 | 964 | 1036 |
| | hypothetical | 202-211, 233-242, 250-259, 280-291, 293-314, | | | | |
| | protein | 318-323, 330-338, 366-379, 381-387, 397-404 | | | | |
| SF3909 | uridine | 4-13, 20-28, 33-49, 61-70, 76-94, 100-146, 151-163, | A: 7 | 12-53 | 965 | 1037 |
| | phosphorylase | 175-182, 202-212, 218-225, 241-252 | | | | |
| SF3939 | histidine | 12-46, 49-64, 81-97, 103-108, 119-140, 150-173, 179-193, | A: 16 | 26-49 | 966 | 1038 |
| | protein kinase | 196-213, 215-223, 225-235, 238-253, 259-264, | | | | |
| | sensor for GlnG | 267-274, 278-284, 292-305, 314-321, 340-346 | | | | |
| | regulator | | | | | |
| SF3969 | formate | 18-23, 28-50, 100-150, 157-186, 197-203, 219-231, | A: 3 | 10-45 | 967 | 1039 |
| | dehydrogenase- | 233-240, 257-280 | | | | |
| | O, iron-sulfur | | | | | |
| | subunit | | | | | |
| SF4060 | RNA | 10-16, 29-49, 59-73, 79-118, 154-169, 178-192, 204-209, | B: 2 | 1054-1156 | 968 | 1040 |
| | polymerase | 216-224, 231-242, 250-256, 269-275, 290-311, | | | | |
| | beta subunit | 319-325, 329-339, 354-365, 371-378, 436-444, 476-485, | | | | |
| | | 507-516, 519-525, 540-547, 556-573, 583-591, | | | | |
| | | 598-604, 606-620, 623-629, 638-648, 653-660, 663-684, | | | | |
| | | 699-704, 707-712, 718-747, 759-765, 777-787, | | | | |
| | | 790-797, 802-808, 826-836, 841-853, 868-876, 879-887, | | | | |
| | | 891-898, 909-916, 922-934, 936-947, 972-994, | | | | |
| | | 1022-1029, 1054-1070, 1084-1090, 1104-1115, 1121-1127, | | | | |
| | | 1146-1155, 1157-1174, 1194-1200, 1207-1215, | | | | |
| | | 1233-1253, 1261-1273, 1286-1307, 1333-1341 | | | | |
| SF4229 | aminopeptidase | 14-26, 28-37, 40-49, 58-78, 89-95, 107-118, 125-132, | C: 13, D: 2 | 113-187 | 969 | 1041 |
| | A-I | 135-142, 155-161, 175-185, 195-213, 215-225, 233-241, | | | | |
| | | 248-254, 263-271, 294-323, 334-340, 345-351, | | | | |
| | | 355-368, 371-394, 401-410, 419-424, 449-456, 463-469, | | | | |
| | | 483-496 | | | | |
| pCP0179 | putative | 10-35, 52-116, 133-143, 149-170, 176-184 | A: 7 | 94-134 | 970 | 1042 |
| | transposase | | | | | |
| pCP0254 | orf, conserved | 4-10, 13-19, 31-39, 57-72, 92-103, 109-122, 126-144, | A: 6, B: 2, | 36-62, 157-180, | 971 | 1043 |
| | hypothetical | 168-174, 179-192, 234-245, 257-262 | C: 28, D: 2 | 190-270 | | |
| | protein (IS | | | | | |
| | Element) | | | | | |
| pCP0262 | Hypothetical | 4-29, 36-52, 60-68, 70-90 | A: 161, | 5-73 | 972 | 1044 |
| | protein | | B: 8 | | | |
| virG | virulent protein | 5-12, 22-56, 58-63, 69-82, 140-146, 175-180, 204-212, | B: 114, | 1-335 | 973 | 1045 |
| | | 240-248, 276-283, 307-312, 324-329, 335-353, 372-385, | D: 95 | | | |
| | | 403-412, 436-443, 448-464, 468-474, 476-483, | | | | |
| | | 503-514, 566-573, 590-597, 601-611, 619-626, 630-639, | | | | |
| | | 647-655, 666-679, 689-697, 707-719, 721-728, | | | | |
| | | 761-769, 783-789, 797-803, 806-812, 845-853, 864-870, | | | | |
| | | 893-904, 917-923, 949-956, 967-985, 1005-1021, | | | | |
| | | 1027-1034, 1059-1065 | | | | |
| ARF0504 | Hypothetical | 4-16, 18-28, 48-55, 67-75, 81-87, 94-100, 108-124, | A: 2 | 110-136 | 974 | 1046 |
| | protein | 139-147, 150-156 | | | | |
| ARF1190 | Hypothetical | 4-22, 28-37, 39-46 | C: 4 | 28-38 | 975 | 1047 |
| | protein | | | | | |
| ARF1280 | Hypothetical | 4-10, 13-19, 27-34, 40-103, 106-113 | A: 2 | 79-100 | 976 | 1048 |
| | protein | | | | | |
| ARF1420 | Hypothetical | none | C: 7 | 11-23 | 977 | 1049 |
| | protein | | | | | |
| ARF3121 | Hypothetical | 4-10, 19-25, 45-51, 64-75, 79-89 | C: 4 | 73-96 | 978 | 1050 |
| | protein | | | | | |
| ARF3760 | Hypothetical | 4-22, 29-36, 38-47 | A: 4, C: 3 | 21-44 | 979 | 1051 |
| | protein | | | | | |
| ARF4075 | Hypothetical | 8-20, 39-44 | A: 4 | 26-42 | 980 | 1052 |
| | protein | | | | | |
| ARF4262 | Hypothetical | 8-19, 31-47, 49-58, 64-79, 84-96 | C: 8 | 16-50 | 981 | 1053 |
| | protein | | | | | |
| CRF0107 | Hypothetical | 20-26, 31-37 | C: 10 | 4-13 | 982 | 1054 |
| | protein | | | | | |
| CRF0109 | Hypothetical | 4-10, 16-32, 34-42, 60-66 | A: 4 | 7-25 | 983 | 1055 |
| | protein | | | | | |
| CRF0548 | Hypothetical | 4-9, 23-45 | A: 7 | 8-44 | 984 | 1056 |
| | protein | | | | | |
| CRF0598 | Hypothetical | 4-19, 34-40, 53-81, 103-117, 122-187 | A: 13 | 22-51 | 985 | 1057 |
| | protein | | | | | |
| CRF1557 | Hypothetical | 4-24 | A: 3 | 1-30 | 986 | 1058 |
| | protein | | | | | |
| CRF2403 | Hypothetical | none | A: 5 | 2-27 | 987 | 1059 |
| | protein | | | | | |
| CRF2748 | Hypothetical | 4-18 | A: 6 | 9-44 | 988 | 1060 |
| | protein | | | | | |
| CRF2911 | Hypothetical | 4-17, 19-26, 41-49, 63-87, 92-99, 113-131 | D: 16 | 6-105 | 989 | 1061 |
| | protein | | | | | |
| CRF3470 | Hypothetical | 6-12 | A: 4 | 9-32 | 990 | 1062 |
| | protein | | | | | |
| CRF3528 | Hypothetical | 4-44, 49-62 | A: 5 | 22-65 | 991 | 1063 |
| | protein | | | | | |
| CRF3798 | Hypothetical | 20-28, 34-40 | C: 18 | 1-14 | 992 | 1064 |
| | protein | | | | | |
| CRF3843 | Hypothetical | 4-29, 35-57 | A: 11 | 17-43 | 993 | 1065 |
| | protein | | | | | |
| CRF4047 | Hypothetical | 14-29 | A: 3 | 24-35 | 994 | 1066 |
| | protein | | | | | |
| CRF4125 | Hypothetical | 4-19, 31-47, 62-73, 76-83, 87-93, 99-106 | A: 5 | 10-38 | 995 | 1067 |
| | protein | | | | | |
| CRF4283 | Hypothetical | 4-10, 12-26 | A: 3 | 1-28 | 996 | 1068 |
| | protein | | | | | |

A, 50 bp library of S. flexneri 2a in lamB with IC13-IgG (855),
B, 300 bp library in fhuA with IC13-IgG (812),
C, 50 bp library in lamB with IC14-IgG (745),
D, 300 bp library in fhuA with IC14-IgG (773);
*prediction of antigenic sequences longer than 5 amino acids was performed with the program ANTIGENIC {Kolaskar, A. et al., 1990}.

TABLE 3
Immunogenic proteins identified from C. jejuni by bacterial surface display.

| | | | No. of | Location | | |
|---|---|---|---|---|---|---|
| | | | selected | of | | |
| C. jejuni | | | clones per | identified | Seq. | Seq. |
| antigenic | Putative function | | ORF and | immunogenic | ID | ID |
| protein | (by homology) | predicted immunogenic aa* | screen | region (aa) | (DNA) | (Protein) |
| Cj0005c | putative | 16-30, 38-45, 55-61, 69-75, 131-141, 159-165, | B: 2 | 148-226 | 1069 | 1203 |
| | molybdenum | 169-179, 182-191, 206-214, 230-245, 252-262, | | | | |
| | containing | 272-280, 299-305, 328-339, 362-370, 372-378, | | | | |
| | oxidoreductase | 388-393, 401-409 | | | | |
| Cj0007 | glutamate synthase | 13-18, 24-33, 36-49, 69-79, 93-101, 113-119, 129-136, | B: 3, D: 7 | 1-14; 1115-1214 | 1070 | 1204 |
| | (NADPH) large | 139-164, 167-175, 182-193, 195-217, 224-231, | | | | |
| | subunit | 282-293, 336-345, 351-358, 373-378, 382-387, | | | | |
| | | 407-415, 441-447, 451-459, 461-470, 479-485, | | | | |
| | | 499-526, 533-544, 559-569, 572-581, 583-592, | | | | |
| | | 598-609, 613-624, 632-649, 654-660, 666-694, | | | | |
| | | 700-706, 712-722, 738-752, 771-778, 800-816, | | | | |
| | | 824-839, 847-864, 879-885, 918-925, 927-946, | | | | |
| | | 957-967, 971-999, 1006-1014, 1018-1037, | | | | |
| | | 1046-1055, 1057-1071, 1090-1098, 1104-1113, | | | | |
| | | 1115-1128, 1181-1187, 1193-1202, 1205-1211, | | | | |
| | | 1223-1234, 1240-1248, 1259-1265, 1273-1281, | | | | |
| | | 1289-1301, 1336-1343, 1346-1352, 1356-1363, | | | | |
| | | 1365-1378, 1382-1391, 1401-1408, 1416-1423, | | | | |
| | | 1433-1443, 1450-1455, 1461-1467, 1475-1481, | | | | |
| | | 1486-1493 | | | | |
| Cj0029 | cytoplasmic L- | 6-11, 31-53, 69-84, 86-93, 102-108, 112-121, 135-145, | F: 5 | 199-267 | 1071 | 1205 |
| | asparaginase | 157-177, 185-202, 208-226, 232-241, 244-251, | | | | |
| | | 260-289, 306-316 | | | | |
| Cj0037c | putative | 4-25, 36-41, 44-49, 68-78, 83-89, 95-102, 113-124, | A: 3, B: 7 | 49-119 | 1072 | 1206 |
| | cytochrome c | 137-142, 163-175, 179-185, 209-218, 233-244, | | | | |
| | | 250-261, 279-288, 308-322, 330-336 | | | | |
| Cj0079c | cytolethal | 4-24, 42-50, 57-75, 101-107, 109-131, 153-161, | E: 2 | 197-220; | 1073 | 1207 |
| | distending toxin | 180-190, 214-223, 226-240, 248-265 | | 252-267 | | |
| Cj0093 | putative | 4-24, 56-62, 111-117, 125-130, 138-143, 153-167, | A: 2; F: 7 | 15-111 | 1074 | 1208 |
| | periplasmic | 169-175, 195-204, 207-217, 234-250, 284-298, | | | | |
| | protein | 303-308, 314-326, 351-356, 359-371 | | | | |
| Cj0113 | peptidoglycan | 4-31, 58-64, 74-80, 88-94, 116-127, 131-138, 141-149, | A: 8; F: 2 | 1-63; 105-163 | 1075 | 1209 |
| | associated | | | | | |
| | lipoprotein | | | | | |
| | (omp18) | | | | | |
| Cj0134 | homoserine kinase | 4-9, 15-26, 33-40, 57-68, 91-108, 112-124, 132-152, | E: 1 | 203-222 | 1076 | 1210 |
| | | 158-168, 186-213, 228-236, 253-269, 276-283 | | | | |
| Cj0143c | periplasmic solute | 4-18, 31-48, 50-61, 81-88, 103-110, 114-121, 143-152, | E: 2 | 138-153 | 1077 | 1211 |
| | binding protein for | 154-164, 176-194, 199-210, 217-225, 234-240, | | | | |
| | ABC transporter | 245-254, 264-279 | | | | |
| Cj0158c | putative haem- | 4-20, 35-41, 51-61, 76-88, 107-115, 122-128 | F: 5 | 13-108 | 1078 | 1212 |
| | binding | | | | | |
| | lipoprotein | | | | | |
| Cj0178 | putative outer | 4-18, 25-31, 41-46, 61-70, 79-85, 126-133, 135-141, | D: 6 | 157-219, | 1079 | 1213 |
| | membrane | 166-172, 175-181, 191-199, 208-217, 250-258, | | 432-508 | | |
| | siderophore | 261-273, 312-320, 370-376, 385-394, 401-409, | | | | |
| | receptor | 411-416, 431-445, 457-478, 531-543, 560-567, | | | | |
| | | 575-593, 607-615, 625-633, 679-689, 733-740, | | | | |
| | | 746-752 | | | | |
| Cj0181 | possible tonB | 4-28, 47-53, 58-64, 88-97, 124-129, 136-141, 146-167, | E: 4 | 64-92 | 1080 | 1214 |
| | transport protein | 174-184, 187-193, 195-203, 205-220, 231-237 | | | | |
| Cj0264c | molybdopterin- | 7-39, 44-56, 60-65, 83-99, 117-132, 142-148, 162-179, | E: 2, F: 5 | 55-76, 667-736 | 1081 | 1215 |
| | containing | 185-203, 213-231, 239-245, 248-259, 288-295, | | | | |
| | oxidoreductase | 330-335, 370-381, 391-396, 401-407, 443-462, | | | | |
| | | 472-492, 505-512, 521-530, 549-557, 562-577, | | | | |
| | | 647-653, 660-666, 673-680, 695-703, 710-717, | | | | |
| | | 729-734, 745-751, 756-766, 769-778, 787-794, | | | | |
| | | 812-819, 825-834 | | | | |
| Cj0281c | putative | 5-11, 21-27, 48-56, 64-73, 75-85, 110-117, 124-130, | B: 5 | 100-124 | 1082 | 1216 |
| | transaldolase | 132-138, 145-163, 165-171, 186-198, 231-239, | | | | |
| | | 246-254, 265-270, 289-296, 312-322 | | | | |
| Cj0285c | chemotaxis protein | 16-24, 26-63, 66-77, 91-97, 100-111, 148-169, | B: 1 | 252-296 | 1083 | 1217 |
| | | 182-188, 205-212, 223-230, 235-244, 264-272, | | | | |
| | | 291-297, 305-312 | | | | |
| Cj0297c | pantoate-beta- | 4-17, 19-31, 34-44, 47-59, 87-93, 99-105, 113-119, | D: 16 | 85-152 | 1084 | 1218 |
| | alanine ligase | 124-137, 139-146, 150-163, 165-175, 185-191, | | | | |
| | | 197-214, 222-227, 235-246, 257-270, 274-279 | | | | |
| Cj0396c | putative | 4-20, 40-52, 74-81, 110-117, 123-138, 144-150, | C: 7 | 33-55 | 1085 | 1219 |
| | lipoprotein | 163-181, 185-195, 199-232, 234-248, 269-277, | | | | |
| | | 280-286, 301-314, 324-329 | | | | |
| Cj0406c | putative | 4-19, 33-55, 67-74, 78-84, 91-105, 109-117, 132-138, | B: 1 | 151-162 | 1086 | 1220 |
| | lipoprotein | 167-176, 190-196, 202-207, 210-217 | | | | |
| Cj0415 | putative | 4-14, 20-26, 28-37, 77-84, 89-94, 114-123, 174-180, | E: 11 | 77-110 | 1087 | 1221 |
| | oxidoreductase | 200-208, 222-239, 241-251, 263-271, 276-282, | | | | |
| | subunit | 291-302, 330-335, 374-390, 392-400, 432-441, | | | | |
| | | 447-454, 462-467, 487-501, 516-526, 530-537, | | | | |
| | | 550-566 | | | | |
| Cj0432c | UDP-N- | 16-23, 25-31, 42-48, 57-75, 81-95, 108-118, 124-151, | B: 2 | 89-142 | 1088 | 1222 |
| | acetylmuramoylalanine- | 153-170, 178-185, 190-201, 216-222, 230-260, | | | | |
| | D-glutamate | 290-297, 300-307, 312-318, 326-334, 348-363, | | | | |
| | ligase | 365-385 | | | | |
| Cj0448c | putative MCP-type | 41-50, 52-70, 80-91, 123-131, 136-143, 146-158, | F: 4 | 59-203 | 1089 | 1223 |
| | signal transduction | 167-178, 207-213, 228-235, 242-264, 274-288, | | | | |
| | protein | 302-307, 335-347, 349-355 | | | | |
| Cj0505c | putative | 5-14, 21-31, 42-71, 76-103, 111-119, 123-132, | B: 2 | 335-347 | 1090 | 1224 |
| | aminotransferase | 138-144, 167-188, 194-200, 205-212, 230-246, | | | | |
| | (degT family) | 253-260, 263-269, 275-294, 300-319, 330-343, | | | | |
| | | 349-354 | | | | |
| Cj0543 | prolyl-tRNA | 6-14, 18-57, 67-81, 87-97, 104-109, 115-122, 125-139, | E: 9 | 410-444 | 1091 | 1225 |
| | synthetase | 147-156, 178-188, 193-199, 211-216, 221-235, | | | | |
| | | 265-287, 293-300, 312-323, 330-352, 369-379, | | | | |
| | | 385-390, 392-404, 408-420, 437-457, 471-483, | | | | |
| | | 489-494, 501-509, 525-535, 547-554 | | | | |
| Cj0548 | putative flagellar | 4-18, 55-82, 87-100, 105-117, 145-150, 168-175, | A: 15; | 99-226; | 1092 | 1226 |
| | hook-associated | 183-189, 198-203, 228-235, 247-256, 277-283, | B: 2; F: 12 | 566-643 | | |
| | protein | 293-301, 308-321, 337-362, 373-379, 392-397, | | | | |
| | | 427-434, 457-463, 502-510, 539-552, 560-566, | | | | |
| | | 615-629 | | | | |
| Cj0596 | major antigenic | 4-27, 59-75, 83-88, 95-105, 112-120, 128-144, | A: 3; F: 2 | 1-103; 124-232 | 1093 | 1227 |
| | peptide PEB3\cell | 164-169, 200-205, 220-226, 237-242, 253-259, | | | | |
| | binding factor 2 | 264-270 | | | | |
| Cj0599 | putative | 12-42, 69-77, 103-109, 120-128, 149-157, 161-166, | E: 3 | 138-216; | 1094 | 1228 |
| | periplasmic | 179-197, 201-218, 238-248, 253-261, 266-272, | | 272-287 | | |
| | protein | 278-286, 307-314 | | | | |
| Cj0613 | possible | 4-26, 28-43, 50-56, 62-67, 103-109, 121-130, 155-163, | A: 5 | 41-91; 179-200 | 1095 | 1229 |
| | periplasmic | 173-179, 197-202, 208-215, 221-233, 258-267, | | | | |
| | phosphate binding | 296-305, 308-314, 321-328 | | | | |
| | protein | | | | | |
| Cj0631c | putative | 4-15, 23-32, 48-55, 61-67, 86-98, 102-111, 124-130, | B: 4 | 475-513 | 1096 | 1230 |
| | ribonuclease | 137-157, 166-171, 178-186, 193-200, 218-232, | | | | |
| | | 236-241, 246-264, 274-288, 291-310, 338-346, | | | | |
| | | 348-359, 389-395, 402-414, 416-428, 430-443, | | | | |
| | | 445-451, 455-469, 473-484, 489-499, 501-509, | | | | |
| | | 511-525, 534-542, 556-561, 579-593, 601-621, | | | | |
| | | 625-637 | | | | |
| Cj0642 | putative DNA | 23-30, 33-50, 53-59, 106-114, 126-132, 140-146, | D: 13 | 88-176 | 1097 | 1231 |
| | repair protein | 171-180, 196-204, 224-240, 242-254, 262-272, | | | | |
| | | 274-282, 288-296, 326-332, 341-352, 378-387, | | | | |
| | | 394-407, 412-418, 431-464, 489-496 | | | | |
| Cj0652 | penicillin-binding | 4-35, 43-52, 60-79, 93-100, 120-139, 146-154, | F: 3 | 488-559 | 1098 | 1232 |
| | protein | 157-171, 208-226, 234-247, 265-271, 273-283, | | | | |
| | | 292-298, 309-323, 330-339, 355-382, 396-409, | | | | |
| | | 445-453, 455-465, 484-505, 512-525, 528-535, | | | | |
| | | 547-576, 584-594 | | | | |
| Cj0683 | putative | 5-28, 34-41, 47-54, 79-100, 112-117, 122-127, | F: 3 | 43-96 | 1099 | 1233 |
| | periplasmic | 130-139 | | | | |
| | protein | | | | | |
| Cj0687c | putative flagellar | 4-23, 25-38, 45-50, 68-77, 105-113, 149-156, 177-188, | F: 2 | 84-145 | 1100 | 1234 |
| | L-ring protein | 197-208, 222-229 | | | | |
| | precursor | | | | | |
| Cj0689 | acetate kinase | 11-16, 19-32, 37-43, 62-69, 72-80, 82-93, 97-128, | E: 3 | 6-26; 347-362 | 1101 | 1235 |
| | | 133-142, 156-166, 179-193, 199-205, 209-216, | | | | |
| | | 239-256, 269-278, 295-325, 332-349, 369-379 | | | | |
| Cj0690c | possible restriction/ | 4-10, 17-28, 39-48, 67-82, 93-100, 122-128, 144-153, | F: 2 | 934-1001 | 1102 | 1236 |
| | modification | 177-184, 196-208, 263-285, 289-295, 297-310, | | | | |
| | enzyme | 353-359, 367-377, 389-409, 413-421, 426-433, | | | | |
| | | 436-442, 445-451, 453-459, 468-475, 477-488, | | | | |
| | | 506-526, 544-554, 562-611, 639-650, 678-692, | | | | |
| | | 697-706, 718-724, 733-744, 746-754, 766-782, | | | | |
| | | 789-796, 808-822, 831-843, 847-856, 861-868, | | | | |
| | | 887-902, 931-937, 939-951, 957-964, 970-976, | | | | |
| | | 997-1003, 1017-1024, 1033-1039, 1048-1054, | | | | |
| | | 1060-1066, 1075-1083, 1092-1130, 1136-1152, | | | | |
| | | 1168-1176, 1210-1221, 1228-1238, 1242-1247 | | | | |
| Cj0692c | putative | 73-86, 88-95, 113-118, 166-178, 223-230, 243-280 | B: 1 | 202-216 | 1103 | 1237 |
| | membrane protein | | | | | |
| Cj0696 | cell division | 16-22, 40-46, 51-62, 77-83, 93-106, 116-124, 127-138, | F: 10 | 50-127 | 1104 | 1238 |
| | protein ftsZ | 151-158, 162-177, 185-207, 211-220, 223-234, | | | | |
| | | 261-269, 271-280, 309-317, 339-350 | | | | |
| Cj0699c | glutamine | 11-26, 36-42, 74-85, 88-101, 106-121, 137-156, | F: 4 | 95-163 | 1105 | 1239 |
| | synthetase | 186-198, 203-224, 226-254, 295-308, 311-318, | | | | |
| | | 327-355, 373-385, 417-443, 445-464 | | | | |
| Cj0704 | glycyl-tRNA | 5-11, 16-23, 34-43, 48-56, 71-83, 89-96, 101-110, | F: 3 | 19-90 | 1106 | 1240 |
| | synthetase alpha | 138-162, 164-180, 189-195, 204-214, 222-227, | | | | |
| | chain | 229-250, 255-261, 272-278 | | | | |
| Cj0709 | signal recognition | 13-19, 26-53, 67-73, 76-82, 87-102, 112-121, 123-148, | E: 3; F: 9 | 339-446 | 1107 | 1241 |
| | particle protein | 151-156, 162-170, 175-186, 199-209, 211-217, | | | | |
| | | 230-244, 251-287, 300-305, 368-374, 403-415, | | | | |
| | | 418-424 | | | | |
| Cj0718 | DNA polymerase | 4-19, 22-30, 59-73, 84-92, 97-117, 128-140, 165-173, | B: 6, F: 4 | 866-789 | 1108 | 1242 |
| | III, alpha chain | 224-234, 245-255, 276-284, 302-309, 323-330, | | | | |
| | | 349-355, 364-376, 380-385, 391-402, 404-415, | | | | |
| | | 424-432, 437-446, 449-477, 484-499, 516-526, | | | | |
| | | 537-544, 550-555, 562-568, 573-601, 616-623, | | | | |
| | | 653-663, 681-718, 720-728, 762-771, 779-799, | | | | |
| | | 801-806, 814-820, 827-835, 851-870, 899-906, | | | | |
| | | 950-957, 973-990, 995-1005, 1036-1052, | | | | |
| | | 1071-1077, 1086-1098, 1147-1152, 1167-1194 | | | | |
| Cj0720c | flagellin | 27-55, 57-64, 66-75, 135-142, 172-178, 191-197, | B: 6 | 184-217 | 1109 | 1243 |
| | | 229-235, 241-246 | | | | |
| Cj0723c | putative integral | 4-24, 33-39, 55-83, 91-114, 126-132, 137-159, | B: 1 | 113-128 | 1110 | 1244 |
| | membrane zinc- | 164-189, 202-224, 232-238, 241-253, 259-282, | | | | |
| | metalloprotease | 285-308, 310-335, 359-364, 371-388 | | | | |
| Cj0737 | putative | 6-28, 54-59, 103-114, 122-130, 145-153, 167-175, | D: 21 | 10-103 | 1111 | 1245 |
| | periplasmic | 184-190, 193-204, 207-212, 215-223, 282-289, | | | | |
| | protein | 291-300, 315-321, 327-339, 347-354 | | | | |
| Cj0753c | tonB transport | 14-37, 52-57, 73-85, 96-113, 124-136, 142-148, | F: 21 | 64-160 | 1112 | 1246 |
| | protein | 150-157, 166-172, 182-194, 199-215, 217-224 | | | | |
| Cj0755 | putative iron | 4-18, 20-33, 59-77, 93-100, 120-129, 131-137, | E: 4 | 406-431 | 1113 | 1247 |
| | uptake protein | 140-146, 148-158, 166-172, 185-191, 243-249, | | | | |
| | | 253-258, 295-307, 327-333, 345-370, 387-394, | | | | |
| | | 425-432, 483-489, 491-501, 521-527, 538-553, | | | | |
| | | 560-570, 576-582, 629-637, 649-658 | | | | |
| Cj0775c | valyI-tRNA | 9-17, 34-39, 41-59, 71-84, 86-98, 148-169, 177-187, | F: 3 | 664-745; | 1114 | 1248 |
| | synthetase | 194-203, 207-214, 222-228, 235-258, 265-273, | | 793-856 | | |
| | | 286-296, 300-307, 316-328, 338-361, 367-375, | | | | |
| | | 394-401, 407-418, 425-434, 480-495, 502-522, | | | | |
| | | 544-560, 568-575, 584-592, 600-622, 636-641, | | | | |
| | | 661-667, 669-690, 700-707, 719-731, 733-739, | | | | |
| | | 745-750, 767-776, 784-791, 795-811, 840-846, | | | | |
| | | 853-866 | | | | |
| Cj0802 | cysteinyI-tRNA | 19-54, 86-91, 98-105, 109-124, 126-136, 145-150, | E: 5 | 404-429 | 1115 | 1249 |
| | synthetase | 173-178, 196-204, 212-224, 229-239, 246-252, | | | | |
| | | 279-299, 307-313, 323-329, 337-345, 349-361, | | | | |
| | | 382-393, 399-406, 416-421 | | | | |
| Cj0814 | hypothetical | 20-26, 66-71, 84-97, 105-111, 122-137, 145-165, | F: 2 | 64-158 | 1116 | 1250 |
| | protein | 170-185, 204-210, 230-242 | | | | |
| Cj0832c | putative integral | 4-16, 28-69, 72-85, 87-99, 101-123, 128-136, 140-159, | B: 4 | 117-132 | 1117 | 1251 |
| | membrane protein | 161-173, 185-205, 207-220, 227-240, 242-252, | | | | |
| | | 274-280, 290-296, 301-326, 356-379, 399-453, | | | | |
| | | 461-473, 485-498, 501-525, 527-574 | | | | |
| Cj0849c | hypothetical | 4-11, 37-52, 56-64, 71-82, 89-96, 108-116, 122-137, | E: 1 | 536-549 | 1118 | 1252 |
| | protein | 144-162, 165-182, 184-194, 228-237, 252-267, | | | | |
| | | 289-296, 473-487, 489-503, 511-516, 527-545, | | | | |
| | | 553-570, 584-593, 604-611, 629-638, 640-649, | | | | |
| | | 684-696 | | | | |
| Cj0887c | putative flagellin | 21-37, 81-97, 119-127, 130-143, 158-163, 175-180, | A: 2, B: 5 | 168-235 | 1119 | 1253 |
| | | 219-230, 245-256, 265-273, 293-299, 319-327, | | | | |
| | | 425-434, 472-485, 493-498, 508-513, 552-557, | | | | |
| | | 562-570, 574-580, 588-594, 597-604, 631-636, | | | | |
| | | 640-646, 667-680, 699-718, 725-747 | | | | |
| Cj0942c | preprotein | 4-11, 19-29, 51-56, 65-73, 85-98, 109-121, 125-135, | B: 2 | 700-722 | 1120 | 1254 |
| | translocase SECA | 145-161, 171-177, 204-219, 223-228, 252-258, | | | | |
| | | 262-268, 286-313, 315-325, 327-332, 345-352, | | | | |
| | | 395-410, 429-435, 443-451, 455-463, 465-475, | | | | |
| | | 481-487, 516-522, 549-562, 585-591, 598-605, | | | | |
| | | 607-614, 643-651, 673-682, 690-696, 700-705, | | | | |
| | | 725-734, 738-744, 758-765, 769-779, 781-792, | | | | |
| | | 830-844, 847-853 | | | | |
| Cj0958c | putative | 10-40, 68-91, 95-104, 140-152, 158-170, 185-191, | F: 11 | 31-94 | 1121 | 1255 |
| | membrane protein | 196-201, 205-219, 240-246, 249-256, 262-268, | | | | |
| | | 274-280, 293-313, 315-320, 326-332, 338-358, | | | | |
| | | 402-453, 457-466, 476-516 | | | | |
| Cj1013c | putative | 10-34, 41-47, 50-66, 73-92, 100-107, 121-127, | D: 3 | 277-365 | 1122 | 1256 |
| | membrane protein | 133-139, 146-155, 159-175, 184-191, 223-230, | | | | |
| | | 238-247, 273-286, 300-310, 328-336, 352-358, | | | | |
| | | 365-372, 376-409, 415-423, 446-452, 459-465, | | | | |
| | | 471-484, 509-522, 532-540, 543-554, 586-598, | | | | |
| | | 600-610, 617-632, 670-689, 695-706, 711-727, | | | | |
| | | 741-746, 752-760, 772-835, 843-882, 890-933, | | | | |
| | | 958-973, 991-1022, 1024-1048, 1053-1070 | | | | |
| Cj1031 | putative outer | 4-31, 56-70, 77-96, 112-117, 124-137, 139-155, | B: 1 | 317-353 | 1123 | 1257 |
| | membrane | 160-169, 176-193, 228-234, 237-257, 271-288, | | | | |
| | component of | 317-322, 337-371, 373-391 | | | | |
| | efflux system | | | | | |
| Cj1032 | putative | 4-14, 20-28, 30-52, 54-62, 76-84, 94-100, 125-132, | B: 2 | 181-199 | 1124 | 1258 |
| | membrane fusion | 140-181, 185-191, 208-222 | | | | |
| | component of | | | | | |
| | efflux system | | | | | |
| Cj1033 | putative integral | 9-46, 53-59, 74-80, 82-93, 97-103, 111-117, 130-142, | F: 2 | 243-318 | 1125 | 1259 |
| | membrane | 152-161, 188-197, 236-251, 264-271, 285-295, | | | | |
| | component of | 307-340, 343-394, 397-412, 416-436, 440-472, | | | | |
| | efflux system | 497-527, 540-548, 561-566, 573-583, 591-598, | | | | |
| | | 607-616, 624-631, 639-649, 669-675, 683-689, | | | | |
| | | 694-718, 728-736, 756-771, 782-803, 814-829, | | | | |
| | | 831-871, 873-879, 882-897, 900-912, 920-928, | | | | |
| | | 940-953, 963-998 | | | | |
| Cj1048c | succinyl- | 5-16, 24-30, 34-39, 45-51, 59-72, 80-86, 100-108, | F: 5 | 226-320 | 1126 | 1260 |
| | diaminopimelate | 116-123, 133-140, 148-162, 176-181, 184-211, | | | | |
| | desuccinylase | 219-228, 233-242, 257-278, 300-310, 320-338, | | | | |
| | | 340-345, 352-360 | | | | |
| Cj1061c | isoleucyl-tRNA | 5-12, 51-58, 61-74, 95-112, 124-137, 153-158, | B: 4 | 38-55 | 1127 | 1261 |
| | synthetase | 163-189, 192-204, 209-236, 240-250, 255-273, | | | | |
| | | 304-317, 320-326, 334-348, 350-356, 360-378, | | | | |
| | | 384-416, 439-457, 465-470, 488-493, 496-505, | | | | |
| | | 531-541, 548-557, 579-587, 593-601, 616-647, | | | | |
| | | 649-659, 679-685, 693-702, 705-713, 715-734, | | | | |
| | | 737-743, 751-758, 763-779, 781-788, 791-801, | | | | |
| | | 856-862, 882-896, 903-914 | | | | |
| Cj1073c | ATP-dependent | 8-34, 51-57, 68-76, 79-95, 110-116, 127-140, 142-154, | E: 4, F: 4 | 418-441, | 1128 | 1262 |
| | protease La | 162-172, 174-202, 214-220, 228-239, 292-306, | | 511-591 | | |
| | | 314-320, 322-329, 337-344, 356-371, 374-380, | | | | |
| | | 382-393, 416-421, 424-433, 444-453, 461-468, | | | | |
| | | 470-478, 485-490, 497-503, 511-519, 537-543, | | | | |
| | | 555-564, 566-579, 588-594, 603-609, 615-627, | | | | |
| | | 634-640, 647-659, 663-689, 699-710, 728-735, | | | | |
| | | 739-748, 750-756, 764-769, 776-788 | | | | |
| Cj1076 | putative pyrroline- | 12-19, 21-30, 32-39, 43-49, 55-68, 76-87, 97-104, | B: 11 | 223-239 | 1129 | 1263 |
| | 5-carboxylate | 114-123, 130-141, 153-168, 179-205, 207-216, | | | | |
| | reductase | 218-226 | | | | |
| Cj1126c | putative integral | 8-34, 72-121, 128-141, 153-172, 174-216, 221-256, | B: 1 | 254-268 | 1130 | 1264 |
| | membrane protein | 261-294, 307-315, 332-349, 354-370, 372-435, | | | | |
| | | 452-457, 461-477, 487-492, 503-509, 511-520, | | | | |
| | | 537-549, 559-565, 568-582, 584-591, 593-602, | | | | |
| | | 607-617, 625-638, 655-674, 681-687, 696-703 | | | | |
| Cj1155c | putative cation- | 4-13, 23-40, 79-98, 106-124, 126-149, 154-161, | F: 10 | 522-597 | 1131 | 1265 |
| | transporting | 163-176, 178-187, 199-226, 232-254, 256-276, | | | | |
| | ATPase | 297-304, 308-326, 329-338, 364-373, 384-399, | | | | |
| | | 404-432, 439-499, 502-518, 523-544, 557-568, | | | | |
| | | 571-582, 584-590, 603-617, 621-627, 633-639, | | | | |
| | | 641-651, 653-663, 675-684, 686-699, 705-729, | | | | |
| | | 736-781 | | | | |
| Cj1184c | putative ubiquinol- | 5-45, 49-62, 85-99, 114-122, 136-148, 151-171, | A: 2 | 1-115 | 1132 | 1266 |
| | cytochrome C | 198-211, 253-260, 278-287, 297-303, 309-314, | | | | |
| | reductase | 318-324, 326-336, 348-363 | | | | |
| Cj1229 | putative curved- | 5-13, 22-33, 88-95, 132-144, 160-166, 189-202, | B: 5, E: 2 | 83-98, 244-269 | 1133 | 1267 |
| | DNA binding | 210-223, 253-258, 269-282, 286-294 | | | | |
| | protein | | | | | |
| Cj1233 | putative hydrolase | 18-25, 29-38, 72-95, 97-107, 110-139, 144-152, | B: 4 | 44-56 | 1134 | 1268 |
| | | 155-161, 174-182, 198-203 | | | | |
| Cj1236 | hypothetical | 5-14, 17-23, 29-50, 52-64, 72-98, 109-115, 120-135, | B: 5 | 10-34 | 1135 | 1269 |
| | protein | 137-145, 152-158, 167-175, 178-185, 210-234, | | | | |
| | | 241-255, 258-271, 276-281, 290-303, 307-312 | | | | |
| Cj1262 | two-component | 5-32, 50-56, 62-70, 78-84, 97-121, 132-171, 177-182, | C: 11 | 383-400 | 1136 | 1270 |
| | sensor (histidine | 188-195, 204-214, 241-250, 267-274, 276-281, | | | | |
| | kinase) | 292-309, 311-320, 333-344, 349-361, 375-395, | | | | |
| | | 398-405 | | | | |
| Cj1266c | Ni/Fe-hydrogenase | 4-10, 16-26, 59-80, 82-93, 97-115, 117-131, 148-160, | E: 1, F: 10 | 335-360, | 1137 | 1271 |
| | large subunit | 169-182, 184-210, 217-234, 241-254, 256-263, | | 427-541 | | |
| | | 265-276, 306-312, 344-350, 384-395, 400-409, | | | | |
| | | 416-423, 428-440, 449-465, 496-504, 517-555 | | | | |
| Cj1267c | Ni/Fe-hydrogenase | 4-20, 48-91, 96-115, 134-142, 171-187, 197-217, | D: 7 | 21-130 | 1138 | 1272 |
| | small chain | 222-242, 246-255, 264-270, 277-289, 305-320, | | | | |
| | | 388-352, 354-373 | | | | |
| Cj1272c | putative | 6-23, 25-53, 61-76, 83-92, 107-121, 147-166, 186-201, | C: 136 | 587-614 | 1139 | 1273 |
| | guanosine-3,5- | 207-215, 243-251, 264-274, 282-326, 333-348, | | | | |
| | bis(diphosphate) 3- | 357-366, 371-380, 401-423, 432-465, 471-477, | | | | |
| | pyrophoshatase | 481-490, 500-506, 512-525, 540-560, 583-603, | | | | |
| | | 605-612, 615-626, 647-656, 661-681, 687-693, | | | | |
| | | 713-722 | | | | |
| Cj1290c | biotin carboxylase | 4-9, 15-21, 28-35, 39-54, 59-73, 76-86, 92-108, | F: 18 | 97-175 | 1140 | 1274 |
| | | 120-134, 136-142, 145-161, 209-217, 220-228, | | | | |
| | | 236-249, 258-267, 275-282, 293-304, 306-327, | | | | |
| | | 330-340, 346-352, 354-360, 368-383, 386-392, | | | | |
| | | 401-413 | | | | |
| Cj1316c | hypothetical | 4-11, 20-30, 47-66, 72-97, 108-117, 119-129, 142-163, | E: 6, F: 13 | 301-378 | 1141 | 1275 |
| | protein | 211-219, 224-237, 243-249, 251-263, 270-288, | | | | |
| | | 295-305, 311-316, 326-333, 341-346, 367-375 | | | | |
| Cj1333 | hypothetical | 22-30, 38-45, 62-68, 78-90, 96-103, 107-114, 118-127, | E: 4 | 536-559 | 1142 | 1276 |
| | protein (1318 | 134-148, 150-173, 179-193, 195-200, 205-219, | | | | |
| | family) | 221-234, 239-248, 250-280, 282-296, 308-325, | | | | |
| | | 334-351, 363-389, 425-432, 438-443, 468-481, | | | | |
| | | 488-495, 499-517, 570-593, 602-610, 613-621, | | | | |
| | | 629-637 | | | | |
| Cj1338c | flagellin | 12-23, 26-35, 51-63, 72-78, 86-91, 135-140, 183-190, | A: 14, | 3-131, 232-331 | 1143 | 1277 |
| | | 201-209, 211-219, 241-247, 260-266, 272-281, | B: 2, | | | |
| | | 295-301, 329-335, 339-345, 355-366, 387-402, | D: 10 | | | |
| | | 428-445, 453-459, 503-517, 519-527, 531-538, | | | | |
| | | 546-553, 560-569 | | | | |
| Cj1339c | flagellin | 8-13, 25-35, 51-56, 72-78, 86-91, 135-140, 183-190, | A: 23, | 1-145, 160-183, | 1144 | 1278 |
| | | 201-209, 211-219, 241-247, 260-265, 272-281, | B: 19 | 212-334, | | |
| | | 295-301, 329-335, 339-345, 355-366, 387-402, | | 376-400 | | |
| | | 428-445, 453-459, 503-512, 519-529, 531-538, | | | | |
| | | 546-553, 560-569 | | | | |
| Cj1341c | hypothetical | 5-17, 62-71, 73-116, 118-131, 137-144, 151-158, | E: 2 | 335-348 | 1145 | 1279 |
| | protein (1318 | 160-167, 169-175, 181-190, 193-210, 212-222, | | | | |
| | family) | 231-262, 273-280, 300-329, 341-358, 363-368, | | | | |
| | | 394-400, 403-409, 416-427, 450-457, 464-470, | | | | |
| | | 478-484, 499-511, 513-529, 544-554, 558-565, | | | | |
| | | 573-589, 597-604 | | | | |
| Cj1342c | hypothetical | 4-18, 38-46, 52-60, 65-79, 93-115, 123-131, 144-154, | D: 8 | 159-243 | 1146 | 1280 |
| | protein (617 | 168-183, 191-196, 201-223, 225-236, 250-263, | | | | |
| | family) | 273-278, 289-317, 328-338, 357-373, 384-399 | | | | |
| Cj1346c | putative 1-deoxy- | 11-39, 43-52, 57-69, 72-98, 112-142, 147-154, | F: 2 | 262-323 | 1147 | 1281 |
| | D-xylulose 5- | 159-165, 167-178, 198-210, 213-227, 244-250, | | | | |
| | phosphate | 257-266, 268-286, 295-301, 305-311, 318-338, | | | | |
| | reductoisomerase | 340-346 | | | | |
| Cj1379 | putative | 4-9, 17-23, 40-49, 57-70, 72-87, 92-121, 124-133, | E: 4 | 185-212 | 1148 | 1282 |
| | selenocysteine- | 135-146, 158-164, 173-195, 204-213, 215-221, | | | | |
| | specific elongation | 230-246, 250-257, 260-273, 280-298, 304-309, | | | | |
| | factor | 311-328, 336-343, 362-371, 373-406, 409-418, | | | | |
| | | 433-446, 450-456, 490-496, 503-513, 526-542, | | | | |
| | | 548-563, 569-592 | | | | |
| Cj1380 | putative | 4-17, 23-34, 36-44, 53-62, 72-85, 87-92, 110-115, | B: 4 | 176-201 | 1149 | 1283 |
| | periplasmic | 118-123, 129-150, 155-168, 172-180, 191-197, | | | | |
| | protein | 205-211, 213-223, 225-233 | | | | |
| Cj1422c | possible sugar | 8-26, 33-44, 52-72, 78-96, 145-151, 154-171, 204-212, | E: 2 | 300-322 | 1150 | 1284 |
| | transferase | 223-230, 236-251, 261-272, 280-341, 365-374, | | | | |
| | | 385-394, 417-423, 434-447, 456-486, 494-500, | | | | |
| | | 509-519, 530-546, 556-566, 568-579, 581-603 | | | | |
| Cj1426c | hypothetical | 5-11, 14-21, 26-45, 52-67, 71-79, 82-97, 104-144, | E: 3 | 93-116 | 1151 | 1285 |
| | protein | 151-159, 183-189, 194-205, 211-223, 241-252, | | | | |
| | | 265-273, 275-280 | | | | |
| Cj1463 | hypothetical | 13-20, 55-72, 102-109 | C: 157, | 10-107 | 1152 | 1286 |
| | protein | | D: 37 | | | |
| Cj1466 | putative flagellar | 5-14, 16-22, 38-45, 51-59, 61-78, 94-102, 133-142, | A: 10 | 132-242 | 1153 | 1287 |
| | hook-associated | 153-160, 187-195, 208-221, 240-254, 256-262, | | | | |
| | protein | 270-275, 281-287, 294-299, 338-354, 356-364, | | | | |
| | | 369-378, 446-452, 506-515, 557-564, 576-599 | | | | |
| Cj1474c | putative type II | 4-29, 41-57, 66-75, 86-93, 96-102, 109-116, 124-131, | B: 1 | 250-326 | 1154 | 1288 |
| | protein secretion | 164-171, 188-194, 199-208, 214-231, 289-295, | | | | |
| | system D protein | 305-310, 314-319, 336-351, 362-368, 389-399, | | | | |
| | | 403-412, 422-433, 435-441, 444-461, 463-469 | | | | |
| Cj1478c | outer membrane | 4-16, 46-56, 59-73, 85-94, 97-105, 127-144, 160-166, | B: 1 | 59-76 | 1155 | 1289 |
| | fibronectin- | 188-194, 233-238, 245-250, 270-275, 286-291 | | | | |
| | binding protein | | | | | |
| Cj1484c | putative | 5-33, 58-64, 78-116, 119-127, 139-160, 171-190 | B: 1 | 33-56 | 1156 | 1290 |
| | membrane protein | | | | | |
| Cj1500 | putative integral | 6-11, 19-39, 50-56, 60-66, 83-133, 135-167, 195-216, | B: 1 | 170-191 | 1157 | 1291 |
| | membrane protein | 226-260, 271-319, 327-339, 342-355, 362-368, | | | | |
| | | 373-394 | | | | |
| Cj1506c | putative MCP-type | 4-37, 45-51, 106-119, 132-138, 150-156, 160-171, | F: 38 | 552-658 | 1158 | 1292 |
| | signal transduction | 176-182, 193-203, 207-215, 222-229, 237-244, | | | | |
| | protein | 253-261, 296-372, 403-409, 416-422, 440-449, | | | | |
| | | 451-460, 471-485, 504-510, 538-551, 560-569, | | | | |
| | | 573-582, 585-597, 606-617, 632-646, 658-666, | | | | |
| | | 668-676, 685-694 | | | | |
| Cj1511c | putative formate | 16-32, 53-79, 81-96, 102-111, 124-130, 145-152, | B: 3 | 14-34 | 1159 | 1293 |
| | dehydrogenase | 159-168, 176-189, 206-231, 236-244, 251-258, | | | | |
| | large subunit | 286-292, 304-310, 315-326, 351-361, 383-390, | | | | |
| | | 392-398, 405-411, 430-436, 456-464, 470-480, | | | | |
| | | 482-496, 505-513, 531-540, 546-558, 583-589, | | | | |
| | | 601-607, 621-634, 638-644, 667-673, 681-687, | | | | |
| | | 693-702, 721-733, 737-744, 747-757, 760-767, | | | | |
| | | 772-789, 796-807, 828-823, 846-852, 856-866, | | | | |
| | | 868-880, 882-890, 913-919, 923-929 | | | | |
| Cj1545c | MdaB protein | 19-42, 55-67, 75-95, 144-156, 168-175, 183-189 | B: 2 | 98-109 | 1160 | 1294 |
| | homolog | | | | | |
| Cj1552c | hypothetical | 7-17, 22-27, 34-61, 88-97, 110-117, 152-159, 175-191, | F: 4 | 199-269 | 1161 | 1295 |
| | protein | 202-213, 220-232, 267-285, 296-315, 341-347, | | | | |
| | | 376-392, 400-408, 421-430, 453-462, 464-470, | | | | |
| | | 478-485 | | | | |
| Cj1553c | putative type I | 27-44, 71-80, 114-123, 127-140, 149-167, 175-188, | B: 4 | 3-25 | 1162 | 1296 |
| | restriction enzyme | 191-202, 205-217, 222-227, 270-276, 297-302, | | | | |
| | M protein | 308-318, 324-332, 349-368, 370-376, 382-393, | | | | |
| | | 432-441, 445-459, 472-481, 489-496 | | | | |
| Cj1564 | putative methyl- | 16-49, 57-63, 84-92, 100-120, 124-130, 160-183, | B: 7 | 474-504 | 1163 | 1297 |
| | accepting | 189-200, 202-209, 236-244, 263-280, 283-334, | | | | |
| | chemotaxis signal | 341-346, 377-389, 405-423, 452-458, 485-493, | | | | |
| | transd. protein | 505-513, 518-530, 547-559, 568-579, 595-601, | | | | |
| | | 619-625, 638-649 | | | | |
| Cj1584c | putative peptide | 4-18, 21-30, 43-54, 76-83, 85-106, 116-122, 124-160, | B: 2 | 376-395 | 1164 | 1298 |
| | ABC-transport | 180-185, 198-204, 206-222, 230-242, 258-263, | | | | |
| | system periplasmic | 270-302, 325-332, 359-371, 374-386, 391-402, | | | | |
| | | 411-417, 435-443, 458-485 | | | | |
| Cj1609 | possible sulfate | 17-26, 28-34, 54-62, 83-93, 108-114, 119-125, | E: 2 | 132-153 | 1165 | 1299 |
| | adenylyltransferase | 151-164, 175-187, 197-222, 224-232, 234-250, | | | | |
| | | 265-270, 276-313, 315-342, 373-383 | | | | |
| Cj1622 | putative | 25-42, 47-53, 55-80, 86-113, 116-126, 128-135, | B: 2 | 144-155 | 1166 | 1300 |
| | riboflavin-specific | 151-159, 167-174, 179-188, 207-221, 226-248, | | | | |
| | deaminase | 257-266, 269-279, 297-304, 312-325 | | | | |
| Cj1634c | chorismate | 16-26, 61-71, 75-85, 95-113, 126-163, 175-184, | B: 1 | 57-76 | 1167 | 1301 |
| | synthase | 202-246, 286-291, 293-302, 320-349 | | | | |
| Cj1670c | putative | 4-19, 29-36, 48-59, 92-103, 117-139, 154-164, | B: 3 | 87-113 | 1168 | 1302 |
| | periplasmic | 190-201 | | | | |
| | protein | | | | | |
| Cj1678 | possible | 43-51, 98-108, 123-130, 149-159, 168-179, 182-188, | B: 4, E: 3 | 676-694, | 1169 | 1303 |
| | lipoprotein | 201-207, 231-240, 334-340, 346-354, 369-378, | | 855-881 | | |
| | | 462-472, 493-503, 510-518, 520-538, 565-572, | | | | |
| | | 584-590, 592-598, 616-628, 637-646, 659-675, | | | | |
| | | 688-694, 699-705, 724-732, 740-755, 771-780, | | | | |
| | | 792-801, 827-833, 846-856, 873-880, 882-906 | | | | |
| Cj1694c | 30S ribosomal | 15-45 | E: 2 | 29-60 | 1170 | 1304 |
| | protein S14 | | | | | |
| Cj1718c | 3-isopropylmalate | 4-11, 15-34, 49-77, 92-121, 128-139, 169-176, | B: 2 | 250-277 | 1171 | 1305 |
| | dehydrogenase | 181-210, 216-222, 225-231, 233-246, 248-264, | | | | |
| | | 271-276, 282-306, 319-326, 346-352 | | | | |
| Cj1729c | flagellar hook | 6-20, 46-54, 64-79, 81-87, 131-138, 156-162, 170-178, | D: 8 | 436-529 | 1172 | 1306 |
| | subunit protein | 190-198, 226-232, 254-263, 266-274, 293-303, | | | | |
| | | 313-323, 363-369, 419-424, 426-432, 436-455, | | | | |
| | | 507-513, 515-521, 534-547, 553-559, 596-610, | | | | |
| | | 617-636, 638-647, 659-665, 677-683, 691-696, | | | | |
| | | 710-718, 724-730, 750-755, 770-780, 802-808, | | | | |
| | | 824-842, 857-862 | | | | |
| ARF0021 | Hypothetical | 11-25 | E: 3 | 21-35 | 1173 | 1307 |
| | protein | | | | | |
| ARF0203 | Hypothetical | none | E: 5 | 1-12 | 1174 | 1308 |
| | protein | | | | | |
| ARF0266c | Hypothetical | 4-10 | E: 1 | 2-11 | 1175 | 1309 |
| | protein | | | | | |
| ARF0357c | Hypothetical | 12-20 | B: 5 | 5-14 | 1176 | 1310 |
| | protein | | | | | |
| ARF0585 | Hypothetical | none | E: 3 | 3-10 | 1177 | 1311 |
| | protein | | | | | |
| ARF0781 | Hypothetical | 4-22 | E: 3 | 10-21 | 1178 | 1312 |
| | protein | | | | | |
| ARF1045c | Hypothetical | 4-30, 35-50 | B: 3 | 17-32 | 1179 | 1313 |
| | protein | | | | | |
| ARF1169c | Hypothetical | none | E: 2 | 1-20 | 1180 | 1314 |
| | protein | | | | | |
| ARF1342c | Hypothetical | 16-21 | B: 7 | 7-22 | 1181 | 1315 |
| | protein | | | | | |
| ARF1379 | Hypothetical | 5-13 | B: 3 | 7-18 | 1182 | 1316 |
| | protein | | | | | |
| ARF1410c | Hypothetical | 10-17 | B: 2 | 16-29 | 1183 | 1317 |
| | protein | | | | | |
| ARF1454c | Hypothetical | 17-27 | E: 2 | 22-34 | 1184 | 1318 |
| | protein | | | | | |
| ARF1669c | Hypothetical | 12-18 | E: 2 | 9-21 | 1185 | 1319 |
| | protein | | | | | |
| CRF0036 | Hypothetical | 4-11, 13-94, 100-111, 115-134 | C: 10 | 89-106 | 1186 | 1320 |
| | protein | | | | | |
| CRF0037c | Hypothetical | 4-24 | C: 12 | 10-27 | 1187 | 1321 |
| | protein | | | | | |
| CRF0174c | Hypothetical | 4-14 | B: 15 | 12-23 | 1188 | 1322 |
| | protein | | | | | |
| CRF0337c | Hypothetical | 18-33 | B: 13 | 13-33 | 1189 | 1323 |
| | protein | | | | | |
| CRF0412 | Hypothetical | 16-23, 25-48 | E: 3 | 29-40 | 1190 | 1324 |
| | protein | | | | | |
| CRF0442 | Hypothetical | 4-12, 25-64, 68-76 | D: 61 | 4-61 | 1191 | 1325 |
| | protein | | | | | |
| CRF0631c | Hypothetical | 4-57, 59-66, 96-104 | C: 8 | 76-91 | 1192 | 1326 |
| | protein | | | | | |
| CRF0999c | Hypothetical | 23-43, 45-75, 97-107, 112-121 | F: 35 | 26-62 | 1193 | 1327 |
| | protein | | | | | |
| CRF1009c | Hypothetical | none | C: 12 | 10-24 | 1194 | 1328 |
| | protein | | | | | |
| CRF1059c | Hypothetical | 4-50 | E: 3 | 30-44 | 1195 | 1329 |
| | protein | | | | | |
| CRF1123c | Hypothetical | 4-20, 24-43 | E: 4 | 10-24 | 1196 | 1330 |
| | protein | | | | | |
| CRF1246c | Hypothetical | 4-49, 56-66 | D: 15 | 5-54 | 1197 | 1331 |
| | protein | | | | | |
| CRF1257c | Hypothetical | 33-44 | B: 8 | 15-38 | 1198 | 1332 |
| | protein | | | | | |
| CRF1287c | Hypothetical | 4-36, 39-50 | E: 3 | 32-47 | 1199 | 1333 |
| | protein | | | | | |
| CRF1502c | Hypothetical | 4-9, 16-30, 32-38 | B: 8 | 15-32 | 1200 | 1334 |
| | protein | | | | | |
| CRF1567c | Hypothetical | 4-9 | B: 8 | 27-42 | 1201 | 1335 |
| | protein | | | | | |
| CRF1582c | Hypothetical | 4-16, 32-43, 49-58, 64-72 | E: 4 | 14-27 | 1202 | 1336 |
| | protein | | | | | |

A, 50 bp library of C. jejuni NCTC 11168 in LamB with P12-IgG (628),
B, 50 bp library in LamB with P12-IgG (705),
C, 50 bp library in LamB with P12-IgA (691),
D, 300 bp library in FhuA with P12-IgA (464),
E, 50 bp library in lamB with IC11-IgG (698),
F, 300 bp library in FhuA with IC11-IgG (815),
*prediction of antigenic sequences longer than 5 amino acids was performed with the program ANTIGENIC {Kolaskar, A. et al., 1990}.

TABLE 4
Immunogenic proteins identified from enteroaggregative E. coli (EAEC) by bacterial surface display.

| Antigenic | Homolog | | Seq. | Seq. |
|---|---|---|---|---|
| protein in | in E. coli | | ID | ID |
| EAEC | O157:H7 | predicted immunogenic aa* | (DNA) | (Protein) |
| EAEC32 | ECs0014 | 12-19, 24-29, 37-43, 47-53, 65-72, 83-95, 112-122, 136-147, 162-168, 174-181, | 601 | 763 |
| | | 189-195, 201-208, 216-221, 234-243, 270-276, 278-288, 305-316, 318-342, | | |
| | | 350-356, 368-400, 420-428, 434-443, 471-477, 481-488, 530-535, 540-547, | | |
| | | 566-575, 591-601, 603-609, 624-629 | | |
| EAEC33 | ECs0067 | 11-22, 28-34, 40-45, 65-86, 99-107, 115-125, 132-141, 143-150, 158-190, | 602 | 764 |
| | | 203-211, 216-239, 246-257, 259-270, 272-279, 286-306, 313-332, 338-364, | | |
| | | 371-380, 389-397, 410-418, 422-435, 467-510, 515-521, 532-538, 547-563 | | |
| EAEC34 | ECs0089 | 7-20, 28-45, 51-66, 81-104, 108-115, 124-137, 149-155, 161-206, 209-214, | 603 | 765 |
| | | 222-239, 250-262, 274-282, 309-343, 351-363, 365-386, 405-413, 435-440, | | |
| | | 446-454, 458-466, 470-477, 482-492 | | |
| EAEC35 | ECs0152 | 10-25, 29-35, 39-46, 54-71, 82-88, 102-111, 122-137, 139-145, 152-160, | 604 | 766 |
| | | 162-172, 176-182, 193-201, 209-218, 226-232, 242-249, 258-268, 299-314, | | |
| | | 318-344, 362-376, 393-399, 405-418, 426-463, 473-485, 487-492, 498-503, | | |
| | | 518-544, 561-567 | | |
| EAEC36 | ECs0153 | 16-23, 66-90, 98-110, 125-131, 144-150, 194-200, 213-219, 221-232, 237-256, | 605 | 767 |
| | | 263-281, 293-298, 311-318, 326-337, 339-354, 373-389, 396-402, 404-421, | | |
| | | 427-439, 441-448, 452-462, 467-479, 508-530, 534-541, 544-550, 562-569, | | |
| | | 575-581, 583-592, 595-628, 636-656, 658-672, 674-680, 687-697, 715-721 | | |
| | | 731-736, 739-749, 754-761, 771-788, 790-797, 813-824 | | |
| EAEC37 | ECs0155 | 14-42, 51-57, 66-77, 84-96, 103-111, 129-148, 158-193, 198-208, 212-222, | 606 | 768 |
| | | 242-262 | | |
| EAEC38 | ECs0156 | 4-23, 29-62, 65-84, 98-104, 128-135, 144-161, 167-173, 175-204, 219-240, | 607 | 769 |
| | | 250-264, 266-278, 280-290 | | |
| EAEC39 | ECs0174 | 13-26, 33-45, 50-60, 75-81, 97-105, 123-131, 138-145, 158-166, 168-177 | 608 | 770 |
| EAEC40 | ECs0218 | 6-20, 23-44, 50-61, 67-82, 84-91, 104-125, 133-144, 149-156, 159-166, 173-180, | 609 | 771 |
| | | 182-196, 200-206, 224-239, 245-288, 320-339, 342-349, 359-368, 377-385, | | |
| | | 411-419, 427-435, 453-474, 481-489, 491-497, 505-514, 516-522, 536-564, | | |
| | | 579-586, 618-631, 644-650, 654-661, 663-677, 679-689, 700-705, 710-753, | | |
| | | 795-801, 809-814, 821-827, 842-851, 867-905, 920-925, 931-949, 954-961, | | |
| | | 1017-1022, 1034-1040, 1047-1057, 1062-1075, 1081-1086, 1107-1117, | | |
| | | 1119-1126, 1145-1154, 1162-1172 | | |
| EAEC41 | ECs0314 | 13-21, 47-57, 72-83, 97-102, 105-119, 125-133, 146-153, 170-177, 221-239, | 610 | 772 |
| | | 245-273, 283-291, 299-305, 317-329, 335-343, 358-367, 374-380, 399-407, | | |
| | | 430-438, 449-454, 473-479, 433-505, 517-527, 531-537, 555-560, 586-599, | | |
| | | 601-616, 623-629, 639-647, 649-654, 658-667, 669-676, 690-709, 714-729 | | |
| EAEC42 | ECs0315 | 14-28, 34-40, 45-54, 69-76, 78-83, 86-100, 116-123, 135-143, 146-161, 168-179, | 611 | 773 |
| | | 187-200, 204-225, 236-250, 255-265, 271-292, 298-314 | | |
| EAEC43 | ECs0320 | 4-28, 36-42, 78-85, 106-122, 130-136, 144-150, 161-175, 180-190, 194-200, | 612 | 774 |
| | | 226-234, 256-265, 274-294, 309-316, 324-333, 336-344, 373-379, 382-389, | | |
| | | 398-404, 407-416, 422-446, 451-462, 530-541 | | |
| EAEC44 | ECs0322 | 4-15, 17-42, 71-77, 80-86, 90-116, 123-135, 144-150, 153-163, 183-194 | 613 | 775 |
| EAEC45 | ECs0336 | 7-13, 22-42, 56-62, 84-90, 102-112, 121-133, 140-148, 158-167, 173-181, | 614 | 776 |
| | | 192-199, 227-234, 284-293, 301-307, 336-343, 345-353, 366-372, 376-397, | | |
| | | 400-436, 439-450, 467-478, 504-510, 519-530, 532-547, 551-558, 564-575, | | |
| | | 592-598, 619-630, 636-642, 655-661, 663-669, 671-679, 697-718, 724-736, | | |
| | | 738-752, 759-773, 776-788, 805-823, 827-833, 842-852, 859-864, 874-881, | | |
| | | 883-889, 905-914, 932-939, 941-957, 963-969, 978-991, 1011-1025, 1052-1062, | | |
| | | 1067-1073, 1080-1088, 1106-1112, 1121-1132, 1139-1152 | | |
| ETEC47 | ECs0345 | 9-23, 32-38, 44-54, 64-71, 95-126, 146-154, 163-173, 190-196, 199-206, | 615 | 777 |
| | | 249-256, 262-277, 291-302, 350-356, 380-398, 406-413, 420-430, 433-444, | | |
| | | 471-478, 492-507 | | |
| EAEC46 | ECs0453 | 5-26, 57-66, 68-74, 81-87, 105-116, 122-132, 144-160, 185-212, 217-223, | 616 | 778 |
| | | 228-234, 241-252, 269-274, 291-313, 318-328, 335-342, 368-375, 397-404, | | |
| | | 411-422, 431-439, 462-472, 474-485, 493-499, 509-519, 521-527, 530-558, | | |
| | | 563-579, 590-602 | | |
| EAEC47 | ECs0454 | 9-23, 32-38, 44-54, 64-71, 95-126, 146-154, 163-173, 190-196, 199-206, | 617 | 779 |
| | | 249-256, 262-277, 291-302, 350-356, 380-398, 406-413, 420-430, 433-444, | | |
| | | 471-478, 492-507 | | |
| EAEC48 | ECs0541 | 18-31, 34-53, 57-67, 74-81, 90-106, 136-144, 147-153, 157-163, 170-182, | 618 | 780 |
| | | 192-207, 233-241, 245-251, 256-267, 274-281, 284-306, 318-330, 333-340, | | |
| | | 345-351, 356-379, 388-404, 428-439, 455-466, 468-480, 488-505, 515-526, | | |
| | | 553-564, 571-577, 594-600, 607-614, 616-628, 634-642, 644-651, 655-666, | | |
| | | 672-678, 686-703, 732-738, 745-751, 755-768, 772-778, 785-805, 807-814, | | |
| | | 817-825, 831-853, 858-868, 890-905, 918-926, 934-942, 957-970, 972-981, | | |
| | | 990-1001, 1057-1067, 1069-1077, 1089-1109, 1116-1130, 1133-1141, 1154-1165, | | |
| | | 1190-1206, 1208-1215, 1217-1225, 1228-1254, 1256-1263, 1271-1279, | | |
| | | 1283-1305, 1333-1339, 1357-1367, 1373-1379, 1388-1405, 1432-1442, | | |
| | | 1444-1451, 1453-1461, 1463-1479, 1488-1504, 1516-1524, 1532-1540, | | |
| | | 1555-1568, 1589-1600, 1607-1613, 1633-1638, 1655-1665, 1687-1704, | | |
| | | 1721-1728, 1731-1737, 1744-1763, 1793-1804, 1816-1823, 1833-1850, | | |
| | | 1855-1865, 1868-1875, 1886-1902, 1916-1925, 1931-1937, 1954-1967, | | |
| | | 1971-1977, 1987-2002, 2009-2015, 2030-2036, 2043-2049, 2053-2077, | | |
| | | 2085-2098, 2114-2122, 2129-2136, 2154-2167, 2187-2203, 2232-2243, | | |
| | | 2253-2274, 2286-2303, 2311-2323, 2344-2365, 2371-2378, 2388-2404, | | |
| | | 2406-2413, 2415-2424, 2426-2450, 2454-2461, 2469-2475, 2486-2505 | | |
| EAEC50 | ECs0548 | 4-22, 24-30, 33-39, 60-72, 83-89, 128-137, 153-161, 165-174, 186-194, 212-218, | 619 | 781 |
| | | 251-260, 275-284, 288-297, 309-318, 335-341, 374-383, 399-407, 411-420, | | |
| | | 432-440, 467-482, 518-526, 572-579, 595-609, 649-663, 680-686, 691-702, | | |
| | | 708-714 | | |
| EAEC51 | ECs0600 | 11-25, 39-57, 69-94, 100-107, 118-155, 158-171, 189-201, 226-233, 236-245, | 620 | 782 |
| | | 249-262, 287-296, 298-312, 315-329, 333-342, 351-359, 364-374, 382-388, | | |
| | | 399-407, 411-417, 419-449, 454-471, 486-492, 494-504, 515-541, 547-552, | | |
| | | 582-600, 611-623, 625-641, 651-657, 678-692, 699-709, 713-720, 746-752, | | |
| | | 772-781, 791-804, 829-844, 880-893, 900-910, 915-923, 936-942, 953-970 | | |
| EAEC52 | ECs0689 | 5-11, 17-24, 26-32, 36-43, 50-61, 67-73, 91-102, 111-126, 133-148, 154-161, | 621 | 783 |
| | | 167-173, 179-195, 208-223, 230-240, 242-253, 270-286, 292-306, 308-347, | | |
| | | 352-371, 373-380, 386-395, 404-410, 418-433, 436-444, 447-460, 463-477, | | |
| | | 486-492, 522-533, 548-553 | | |
| EAEC53 | ECs0819 | 4-12, 15-27, 35-55, 68-95, 100-109, 117-122, 129-135, 157-162 | 622 | 784 |
| EAEC54 | ECs0872 | 25-46, 59-65, 68-75, 83-90, 93-100, 107-115, 124-135, 151-177, 183-189, | 623 | 785 |
| | | 194-206, 209-215, 219-224, 251-263, 267-276, 305-311, 318-327, 332-338, | | |
| | | 350-356, 380-396, 406-412, 414-423, 431-437, 453-461, 463-481, 483-491, | | |
| | | 505-510, 513-523, 528-545, 568-575 | | |
| EAEC55 | ECs0883 | 10-35, 42-59, 65-70, 76-85, 92-104, 149-155, 184-191, 234-243, 248-259, | 624 | 786 |
| | | 268-277, 279-287, 391-398, 410-430, 445-454, 488-494, 498-504, 518-523, | | |
| | | 530-538, 574-590, 615-623, 627-633, 652-660, 662-670, 674-683, 703-714, | | |
| | | 720-728, 731-737, 751-757 | | |
| EAEC56 | ECs0931 | 5-12, 39-51, 57-64, 67-84, 86-108, 124-130, 139-159, 167-179, 181-202, | 625 | 787 |
| | | 226-235 | | |
| EAEC57 | ECs0954 | 12-20, 29-40, 57-77, 79-88, 97-103, 111-117, 119-137, 174-200, 202-218, | 626 | 788 |
| | | 221-229, 231-238, 240-246, 254-264, 266-280, 296-308, 321-331 | | |
| EAEC58 | ECs0987 | 4-18, 20-48, 54-68, 80-105, 110-117, 120-130, 132-167, 179-214, 227-246, | 627 | 789 |
| | | 259-295, 306-323, 332-339, 345-351, 357-363, 366-374, 379-392 | | |
| EAEC59 | ECs1044 | 8-21, 23-31, 53-66, 69-94, 99-113, 119-184, 190-214, 233-244, 268-274, | 628 | 790 |
| | | 279-284, 289-298, 300-311, 315-337, 344-350, 364-383, 385-397 | | |
| EAEC60 | ECs1433 | 26-38, 43-63, 67-76, 79-98, 105-112, 115-121, 132-144, 148-153, 179-184, | 629 | 791 |
| | | 194-203, 239-245, 261-278, 282-315 | | |
| EAEC61 | ECs1462 | 13-22, 24-30, 49-61, 65-72, 90-97, 99-105, 115-131, 152-160, 165-171, 176-188, | 630 | 792 |
| | | 202-221, 231-250, 255-274, 280-286, 288-296, 331-337, 339-347, 350-358, | | |
| | | 374-385, 391-408, 418-427, 438-453, 468-476, 482-490, 497-506, 526-532, | | |
| | | 534-583, 696-702, 713-719, 730-748, 750-758, 762-778, 802-808, 825-857, | | |
| | | 864-950, 963-1004, 1015-1023, 1046-1058 | | |
| EAEC62 | ECs1643 | 4-10, 22-28, 35-44, 58-66, 74-84, 86-98, 116-131, 138-143, 181-186, 224-230, | 631 | 793 |
| | | 241-253, 282-292, 305-313, 325-331, 333-341, 348-360, 384-391, 395-408, | | |
| | | 415-429, 431-445, 525-531, 558-564, 567-584, 601-612, 624-637, 645-652, | | |
| | | 682-687, 700-706 | | |
| EAEC63 | ECs1650 | 4-13, 19-27, 33-40, 57-82, 107-115, 117-125, 162-173, 197-206, 215-226, | 632 | 794 |
| | | 256-266, 291-298, 303-310, 318-323, 331-336, 345-360, 379-396, 405-410, | | |
| | | 426-434, 454-462, 468-476, 482-497, 512-518, 529-541, 556-564, 570-581, | | |
| | | 589-594, 601-610, 629-637, 639-648, 663-670, 704-711, 725-738, 746-768, | | |
| | | 770-780, 787-804, 817-823, 830-837, 876-882, 930-936, 939-968 | | |
| EAEC64 | ECs1736 | 5-13, 20-65, 67-74, 107-115, 128-169, 171-195, 238-244, 256-287, 292-298 | 633 | 795 |
| EAEC65 | ECs1752 | 7-13, 16-77, 89-96, 104-114, 117-125, 148-160, 167-191, 193-202, 227-236 | 634 | 796 |
| EAEC66 | ECs1905 | 21-36, 41-47, 54-89, 122-129, 138-165, 173-190, 196-216, 221-229 | 635 | 797 |
| EAEC67 | ECs2026 | 8-45, 135-140, 172-182, 189-196, 206-216, 218-235, 260-269, 272-278, | 636 | 798 |
| | | 307-313, 333-344, 352-359, 371-395, 403-414, 416-422, 426-438, 451-470, | | |
| | | 478-484, 493-502, 504-511, 514-533 | | |
| EAEC68 | ECs2036 | 6-25, 49-59, 65-96, 107-115, 117-124, 135-151, 176-185, 203-209 | 637 | 799 |
| EAEC69 | ECs2037 | 5-15, 46-56, 58-81, 83-111, 118-138, 152-160, 165-175 | 638 | 800 |
| EAEC70 | ECs2044 | 7-16, 18-24, 36-43, 54-60, 65-73, 88-94, 107-113, 122-128, 134-141, 162-171, | 639 | 801 |
| | | 182-216, 218-235, 249-263, 266-278, 290-301, 308-338 | | |
| EAEC71 | ECs2048 | 4-14, 19-24, 27-36, 38-51, 63-73, 90-96, 102-121, 138-150, 157-174, 176-202, | 640 | 802 |
| | | 212-225, 229-245, 250-258, 261-268, 279-291, 293-310, 319-338, 358-368, | | |
| | | 371-389, 393-398, 404-413, 416-433, 435-442, 458-471 | | |
| EAEC72 | ECs2091 | 17-40, 47-82, 85-93, 101-113, 153-172, 180-186, 190-208, 215-224, 252-261, | 641 | 803 |
| | | 269-279, 283-289, 294-306, 311-328, 397-408, 416-423, 425-437, 492-499, | | |
| | | 513-534, 542-548, 550-555 | | |
| EAEC73 | ECs2362 | 8-17, 29-43, 45-52, 58-69, 87-100, 102-112, 148-163, 172-187, 190-208, | 642 | 804 |
| | | 210-227, 232-239, 245-253, 258-263, 286-299, 313-334, 346-362, 373-388, | | |
| | | 391-410, 417-423, 425-430, 434-446, 457-472, 483-489, 496-502, 518-524, | | |
| | | 537-546, 555-560, 602-610, 637-646, 676-689, 698-704, 706-742, 750-778, | | |
| | | 780-791, 806-842, 864-879, 881-888, 890-899, 901-908, 910-921, 941-947, | | |
| | | 953-959, 967-980, 990-995, 1000-1061, 1073-1079, 1081-1092, 1096-1118, | | |
| | | 1121-1168, 1174-1185, 1195-1209, 1219-1232, 1237-1243, 1250-1274, | | |
| | | 1276-1286, 1302-1319, 1324-1333, 1339-1344, 1349-1361, 1370-1376, | | |
| | | 1418-1427, 1435-1449, 1453-1469, 1473-1478, 1482-1495, 1509-1517, | | |
| | | 1519-1526 | | |
| EAEC74 | ECs2389 | 4-14, 16-32, 42-47, 65-71, 82-109, 128-145, 158-171, 177-191, 197-228, | 643 | 805 |
| | | 230-236 | | |
| EAEC75 | ECs2394 | 16-22, 26-42, 52-72, 75-89, 97-102, 114-147, 154-160, 165-170, 172-200, | 644 | 806 |
| | | 202-229, 231-244, 256-261, 267-278, 286-294, 312-319, 330-336, 340-346, | | |
| | | 360-373, 375-383, 386-396, 420-441, 443-474, 484-491, 496-517, 535-574, | | |
| | | 600-606, 608-624, 636-643, 646-658, 664-687, 692-703, 716-725, 733-750, | | |
| | | 755-764 | | |
| EAEC76 | ECs2467 | 4-14, 24-34, 47-69, 81-90, 98-112, 144-153, 161-169, 189-196, 202-208, | 645 | 807 |
| | | 213-220, 243-249, 256-262, 265-271, 279-286, 299-307, 310-324, 326-345, | | |
| | | 356-369, 397-416, 424-429, 432-441 | | |
| EAEC77 | ECs2527 | 4-9, 14-23, 50-56, 59-68, 77-102, 111-120, 126-152, 161-167, 174-180, 189-202, | 646 | 808 |
| | | 204-228, 237-245, 259-266, 278-285, 300-309 | | |
| EAEC78 | ECs2662 | 23-35, 71-77, 94-100, 134-140, 157-163, 189-195, 211-219, 221-231, 238-244, | 647 | 809 |
| | | 246-253, 263-277, 298-306, 315-321, 337-342, 350-355, 369-377, 389-400, | | |
| | | 408-416, 422-427, 441-449, 465-477, 481-488, 527-532, 534-551 | | |
| EAEC79 | ECs2670 | 14-20, 35-48, 53-63, 71-77, 95-101, 114-121, 123-133, 144-151, 153-160, | 648 | 810 |
| | | 162-170, 190-197, 201-211 | | |
| EAEC80 | ECs2676 | 9-17, 26-46, 65-72, 90-101 | 649 | 811 |
| EAEC81 | ECs2699 | 21-39, 46-53, 68-96, 107-113, 118-124, 126-135, 158-185, 196-202, 204-213, | 650 | 812 |
| | | 219-226, 246-253, 267-275, 277-285, 299-317, 319-338, 404-410, 421-428, | | |
| | | 435-463 | | |
| EAEC82 | ECs2776 | 17-24, 29-40, 47-56 | 651 | 813 |
| EAEC83 | ECs2865 | 17-25, 32-77, 82-91, 100-128, 161-169, 194-207, 211-218, 227-232, 239-245, | 652 | 814 |
| | | 255-260, 278-300, 311-325, 342-356, 382-390, 393-401, 416-460, 467-487, | | |
| | | 491-503, 505-512, 516-532, 551-565, 568-575, 594-601, 610-632, 638-643, | | |
| | | 647-670, 672-685, 699-710, 712-726 | | |
| EAEC84 | ECs3019 | 5-14, 25-46, 49-55, 59-65, 77-85, 98-107, 125-169, 181-186, 223-240, 271-281, | 653 | 815 |
| | | 290-300, 306-313, 315-322, 330-337, 348-359, 370-377, 384-392, 416-424, | | |
| | | 428-445, 456-469, 479-486, 502-510, 518-523, 525-535, 555-575, 578-585, | | |
| | | 605-612, 624-637, 661-685, 693-700, 708-721, 723-729, 744-754, 770-775 | | |
| EAEC85 | ECs3081 | 4-33, 38-52, 92-106, 116-124, 133-138, 142-148, 153-159, 161-168, 245-257, | 654 | 816 |
| | | 282-287, 314-321, 331-336, 355-361, 366-372, 374-390, 396-409, 447-455, | | |
| | | 484-490, 498-504, 511-519, 531-538, 540-545, 574-581, 586-596, 625-631, | | |
| | | 644-655, 668-674, 685-692, 718-723, 728-741, 771-778, 787-797, 801-807, | | |
| | | 819-828, 832-844 | | |
| EAEC86 | ECs3103 | 5-25, 31-39, 72-79, 93-102, 104-110, 122-132, 138-146, 157-189, 192-198, | 655 | 817 |
| | | 205-214, 226-233, 240-248, 269-275, 282-298, 304-310, 313-327, 342-348 | | |
| EAEC87 | ECs3111 | 12-39, 49-55, 59-69, 95-104, 106-111, 116-128, 161-184, 186-217, 229-237, | 656 | 818 |
| | | 240-252, 254-269, 271-278, 311-326, 331-338, 348-356, 364-370, 375-408, | | |
| | | 429-460, 471-482, 484-500, 508-516, 527-536, 539-548, 560-576, 583-605, | | |
| | | 643-655, 662-676, 682-687, 691-697, 703-715, 726-734, 737-746, 757-768, | | |
| | | 778-789, 791-814, 821-826, 834-854, 890-899, 914-925, 947-954, 959-967, | | |
| | | 984-990, 993-1000, 1012-1021, 1039-1044, 1065-1070, 1081-1098, 1136-1142, | | |
| | | 1149-1157, 1165-1170, 1175-1186, 1191-1201, 1225-1265, 1276-1285, | | |
| | | 1292-1300, 1323-1333, 1351-1361, 1366-1372, 1383-1397, 1404-1412, | | |
| | | 1417-1425, 1431-1448, 1468-1473, 1483-1494, 1496-1504, 1506-1530 | | |
| EAEC88 | ECs3114 | 18-53, 64-93, 95-105, 124-135, 143-148, 155-161, 163-171, 184-198, 238-245, | 657 | 819 |
| | | 258-271, 273-284, 287-292, 302-310, 312-320, 322-341, 349-365, 377-403, | | |
| | | 407-414, 417-423, 444-453, 455-469, 471-495, 503-511, 536-557, 579-586, | | |
| | | 588-609, 619-626, 632-638, 643-649, 656-663, 669-680, 682-688, 699-714, | | |
| | | 729-739, 755-761, 768-776, 781-793, 801-815, 821-826, 833-842, 863-869 | | |
| EAEC89 | ECs3126 | 8-15, 24-40, 51-65, 78-89, 102-111, 117-154, 164-177, 181-192, 198-209, | 658 | 820 |
| | | 216-222, 230-237, 241-248, 254-268, 285-293, 298-321, 331-338, 366-373, | | |
| | | 384-389, 392-415, 429-439, 441-451, 453-459, 471-486, 489-501, 524-535 | | |
| EAEC90 | ECs3137 | 10-18, 26-38, 48-54, 60-69, 77-83, 88-95, 119-126, 133-169, 172-185, 193-206, | 659 | 821 |
| | | 214-225, 236-250, 269-275, 278-301, 320-329, 336-341, 347-353, 356-369, | | |
| | | 389-396 | | |
| EAEC91 | ECs3205 | 27-32, 37-50, 68-82, 84-108, 134-145, 149-154, 162-170, 172-182, 194-200, | 660 | 822 |
| | | 205-224, 232-270, 293-299, 312-328 | | |
| EAEC92 | ECs3213 | 8-14, 19-45, 65-75, 82-87, 95-105, 135-149, 154-160, 175-184, 205-226, | 661 | 823 |
| | | 229-244, 249-256, 284-296, 298-304, 321-348 | | |
| EAEC93 | ECs3225 | 4-10, 15-24, 26-53, 55-71, 78-83, 90-112, 128-148, 156-163, 165-179, 203-213, | 662 | 824 |
| | | 228-239, 250-259, 277-285, 292-314, 322-330, 335-340, 345-360, 363-370, | | |
| | | 381-396, 404-409, 416-427 | | |
| EAEC94 | ECs3322 | 20-32, 37-46, 53-65, 75-83, 86-95, 99-105, 121-133, 139-151, 183-190, 199-205, | 663 | 825 |
| | | 216-227 | | |
| EAEC95 | ECs3330 | 9-25, 29-48, 50-100, 102-126, 131-149, 167-173, 210-217, 224-256, 259-265, | 664 | 826 |
| | | 275-292, 295-301, 308-313, 319-335, 337-346, 352-359, 362-382, 393-423, | | |
| | | 436-449, 468-476, 481-487, 492-500, 526-534, 537-548, 560-567, 569-579, | | |
| | | 590-598, 604-613, 629-636, 644-656 | | |
| EAEC96 | ECs3386 | 4-18, 25-31, 33-39, 42-53, 64-92, 97-111, 123-129, 134-146, 165-171, 173-190, | 665 | 827 |
| | | 192-213, 226-239, 251-273, 283-298, 316-324, 339-345, 350-356, 361-376, | | |
| | | 400-408, 418-440, 444-451, 476-481, 503-516, 524-542, 555-563, 581-594, | | |
| | | 607-629, 634-641, 647-670, 711-719, 728-738, 755-765, 772-780, 800-815, | | |
| | | 822-833, 842-852, 860-865, 874-880, 891-913, 926-938, 941-946, 961-978, | | |
| | | 984-990, 1014-1024, 1038-1044, 1052-1092, 1099-1111, 1120-1140, | | |
| | | 1153-1168, 1170-1190, 1193-1210, 1221-1233, 1253-1264, 1268-1274, | | |
| | | 1283-1289, 1295-1300, 1303-1327, 1338-1351, 1362-1368, 1391-1396, | | |
| | | 1410-1416, 1429-1441, 1471-1477, 1483-1513, 1526-1555, 1585-1591, | | |
| | | 1596-1630, 1632-1639 | | |
| EAEC97 | ECs3414 | 10-25, 34-54, 57-67, 77-96, 111-121, 127-139, 151-157, 161-179, 183-198, | 666 | 828 |
| | | 201-219, 233-239, 247-252, 268-276, 283-294, 299-309, 319-324 | | |
| EAEC98 | ECs3415 | 7-15, 20-32, 85-97, 102-109, 117-133, 137-161, 176-184, 186-203, 213-225, | 667 | 829 |
| | | 227-251, 258-274, 280-290, 319-325, 335-364, 377-387, 403-410, 412-421, | | |
| | | 436-454, 458-475, 478-504, 512-521, 541-567, 601-609, 614-622, 635-651, | | |
| | | 657-669, 687-694, 702-708, 717-733, 735-741, 766-786, 788-800, 813-818, | | |
| | | 823-834 | | |
| EAEC99 | ECs3515 | 4-38, 49-69, 75-85, 110-115, 127-134, 167-173, 203-211, 240-245, 258-264, | 668 | 830 |
| | | 293-299, 301-316, 348-354, 356-362, 386-391, 405-410, 456-462, 474-483, | | |
| | | 494-499, 511-516, 523-528, 533-538, 549-557, 579-585, 587-593, 607-612, | | |
| | | 618-625, 627-634, 654-660, 664-670, 682-688, 697-702, 729-736, 749-756, | | |
| | | 783-793, 804-812, 817-829, 862-868, 915-920, 944-950, 954-960, 1000-1031, | | |
| | | 1044-1056, 1069-1077, 1079-1084, 1097-1118, 1139-1146, 1152-1158, | | |
| | | 1165-1176, 1181-1186, 1201-1213, 1257-1263, 1268-1276, 1278-1285, | | |
| | | 1354-1360, 1369-1378, 1386-1396, 1439-1446, 1491-1501, 1526-1548, | | |
| | | 1556-1564 | | |
| EAEC100 | ECs3597 | 4-13, 20-34, 47-53, 58-65, 76-82, 89-106, 139-160, 165-182, 191-205 | 669 | 831 |
| EAEC101 | ECs3736 | 20-40, 42-49, 58-67, 71-80, 95-100, 116-122, 131-142, 145-152, 158-173, | 670 | 832 |
| | | 179-186, 196-202, 229-241, 258-270, 289-300, 302-320, 345-351, 370-382, | | |
| | | 391-404, 455-464, 504-514, 516-527 | | |
| EAEC102 | ECs3745 | 5-14, 26-35, 38-45, 54-60, 63-79, 121-127, 137-145, 152-162, 167-173, 175-183, | 671 | 833 |
| | | 191-202, 218-228, 238-263, 278-295, 303-316, 320-335, 337-345, 359-365, | | |
| | | 382-400 | | |
| EAEC103 | ECs3811 | 4-17, 31-38, 46-61, 68-73, 76-97, 128-139, 150-156, 166-172, 174-182, 184-212, | 672 | 834 |
| | | 219-225, 238-245, 249-262 | | |
| EAEC104 | ECs3821 | 4-24, 31-42, 45-54, 59-71, 83-92, 108-115, 123-130, 149-156, 202-208, 224-233 | 673 | 835 |
| EAEC105 | ECs3824 | 4-16, 25-41, 44-52, 60-66, 73-82, 92-101, 108-114, 133-138, 145-155, 177-185, | 674 | 836 |
| | | 194-202 | | |
| EAEC106 | ECs3827 | 4-9, 21-39, 72-78, 82-88, 99-131, 136-143, 151-162, 164-187, 189-204, 208-216, | 675 | 837 |
| | | 223-229, 232-240, 246-256, 269-283, 288-299, 311-321, 328-335 | | |
| EAEC107 | ECs3843 | 26-33, 39-45, 50-62, 79-85, 87-101, 116-131, 142-152, 154-186, 193-199, | 676 | 838 |
| | | 201-216, 221-243, 266-272, 281-297, 324-330, 335-342, 345-355, 375-383, | | |
| | | 407-413 | | |
| EAEC108 | ECs3923 | 4-22, 27-36, 60-69, 90-98, 107-113, 117-123, 127-134, 137-151, 154-161, | 677 | 839 |
| | | 169-178, 185-192, 202-208, 214-223, 230-239, 245-255, 266-275, 307-317, | | |
| | | 323-337, 339-353, 361-379, 385-391, 393-401, 415-422, 424-429, 434-442, | | |
| | | 444-449, 470-480 | | |
| EAEC109 | ECs3951 | 4-22, 29-34, 37-44, 53-80, 98-110, 127-142, 144-156, 158-165 | 678 | 840 |
| EAEC110 | ECs4037 | 4-12, 14-20, 27-34, 39-47, 51-67, 69-81, 89-97, 105-119, 121-133, 140-149, | 679 | 841 |
| | | 151-161 | | |
| EAEC111 | ECs4049 | 17-24, 34-40, 78-85, 227-233, 294-315, 327-335, 345-351, 354-359, 363-368, | 680 | 842 |
| | | 388-403, 405-411, 413-419, 425-434, 462-472, 480-500, 528-536, 542-560, | | |
| | | 566-573, 579-589, 593-606, 614-646, 651-658, 663-669, 686-726, 734-747, | | |
| | | 754-778, 787-806, 809-825, 827-839, 876-887 | | |
| EAEC112 | ECs4079 | 9-29, 35-40, 49-63, 69-76, 110-134, 141-147, 160-169 | 681 | 843 |
| EAEC113 | ECs4091 | 6-31, 33-47, 53-59, 62-78, 93-98, 105-114, 121-130, 136-169, 172-195, 197-208, | 682 | 844 |
| | | 223-228, 236-267, 277-283, 295-307, 309-325, 330-339, 345-352, 358-370, | | |
| | | 379-391, 419-450, 461-508, 510-519, 521-539, 547-575, 578-587, 589-603, | | |
| | | 612-633, 644-657, 666-678, 694-706, 712-717, 728-742, 759-769, 777-784, | | |
| | | 800-806, 820-838, 841-848, 851-856, 870-876, 887-895, 908-914, 923-940, | | |
| | | 942-953, 969-988 | | |
| EAEC115 | ECs4147 | 12-39, 41-50, 68-74, 87-97, 113-136, 141-156, 167-180, 290-196, 204-223, | 683 | 845 |
| | | 229-235, 247-278 | | |
| EAEC116 | ECs4174 | 10-16, 25-53, 64-74 | 684 | 846 |
| EAEC117 | ECs4233 | 5-43, 46-81, 88-96, 137-142, 163-191, 195-203, 210-235, 241-254, 256-276, | 685 | 847 |
| | | 280-288, 292-305, 307-313, 317-333, 335-343, 347-353, 357-363, 372-381, | | |
| | | 384-389, 399-409 | | |
| EAEC118 | ECs4249 | 8-14, 22-32, 35-46, 57-75, 83-91, 100-106, 108-114, 125-131, 133-142, 157-175, | 686 | 848 |
| | | 186-211, 217-235, 246-361, 367-372, 382-394, 396-405, 414-438, 459-471, | | |
| | | 504-510, 513-535, 538-560 | | |
| EAEC119 | ECs4258 | 8-20, 25-30, 46-62, 67-73, 98-103, 105-114, 119-141, 144-153, 168-178, | 687 | 849 |
| | | 181-193, 198-204, 208-227, 236-242, 249-258, 281-288, 291-306, 327-336, | | |
| | | 340-361, 368-380, 389-409, 417-426, 428-435, 442-453, 468-486, 488-496, | | |
| | | 498-509, 511-523, 540-553, 566-579, 587-603, 629-636, 677-682 | | |
| EAEC120 | ECs4337 | 9-25, 41-61, 68-75, 81-102, 106-141, 158-165, 172-191 | 688 | 850 |
| EAEC121 | ECs4346 | 7-38, 43-58, 67-79, 92-99, 103-111, 118-128, 130-139, 152-165, 170-186, | 689 | 851 |
| | | 192-223, 225-251 | | |
| EAEC122 | ECs4347 | 4-16, 21-36, 38-47, 54-64, 92-103, 117-126, 135-155, 157-200, 202-223, | 690 | 852 |
| | | 231-239, 246-262 | | |
| EAEC123 | ECs4410 | 6-38, 44-57, 62-75, 82-96, 104-109, 115-122, 129-136, 147-160, 185-193, | 691 | 853 |
| | | 200-206, 230-245, 248-269, 274-282, 289-298, 306-314, 321-335, 344-362, | | |
| | | 372-377, 385-394, 422-431, 438-447, 479-505, 529-541, 547-558, 564-571, | | |
| | | 573-579, 606-612, 621-632, 638-649, 656-664, 676-683, 695-704, 711-716, | | |
| | | 728-734, 736-742, 776-781, 783-791, 809-816, 850-856, 866-872, 889-898, | | |
| | | 906-912, 919-926, 944-953, 987-992, 1015-1022, 1024-1038, 1066-1072, | | |
| | | 1097-1105, 1113-1119, 1121-1136, 1147-1154 | | |
| EAEC124 | ECs4412 | 5-14, 27-42, 48-67, 71-83, 85-91, 105-112, 114-135, 139-147, 159-165, 169-185, | 692 | 854 |
| | | 188-195, 199-208, 212-221, 231-253, 264-272, 275-282, 290-303, 309-319, | | |
| | | 324-331, 340-358, 380-405, 419-425, 438-444, 450-463, 468-477, 497-514, | | |
| | | 520-533, 549-556, 568-574, 617-626, 637-643, 661-668, 674-684, 705-713, | | |
| | | 718-733, 735-769 | | |
| EAEC125 | ECs4413 | 5-16, 29-53, 63-71, 74-93, 124-132, 140-192, 199-216, 227-258, 260-268, | 693 | 855 |
| | | 272-282, 285-300, 323-331, 353-368, 389-396, 414-421, 429-441, 448-454, | | |
| | | 459-467, 474-493, 501-508, 516-575, 593-612, 631-661, 665-693, 715-724, | | |
| | | 736-751, 754-765, 771-777, 782-791, 809-820, 823-853 | | |
| EAEC126 | ECs4480 | 51-70, 81-95, 103-110, 117-123, 130-136, 142-160, 174-180, 199-207, 268-274, | 694 | 856 |
| | | 280-296, 347-358, 361-369, 390-396, 401-409, 424-430, 442-448, 455-467, | | |
| | | 501-508, 523-533, 553-560 | | |
| EAEC127 | ECs4629 | 4-10, 35-45, 56-92, 108-116, 127-133, 137-146, 156-186, 194-203, 225-234, | 695 | 857 |
| | | 242-251, 272-283, 285-306, 310-315, 322-330, 358-371, 373-379, 389-396 | | |
| EAEC129 | ECs4674 | 4-32, 38-46, 66-83, 88-95, 110-118, 123-141, 169-180, 200-208, 217-225, | 696 | 858 |
| | | 237-245, 247-261, 263-272, 275-282, 291-302, 310-338 345-353, 360-369, | | |
| | | 371-378, 386-394, 398-413, 416-422, 437-448 | | |
| EAEC130 | ECs4761 | 4-11, 18-26, 31-47, 59-68, 74-92, 98-144, 149-158, 173-180, 200-210, 216-223, | 697 | 859 |
| | | 239-250 | | |
| EAEC131 | ECs4860 | 4-10, 29-56, 93-99, 119-124, 133-140, 159-171, 187-195, 200-214, 221-233, | 698 | 860 |
| | | 249-255, 263-271, 285-291, 310-316 | | |
| EAEC161 | ECs4901 | 29-35, 68-85, 92-99, 107-122, 124-135, 138-144, 146-158, 173-205, 208-227, | 699 | 861 |
| | | 232-259, 277-285, 317-324, 326-333 | | |
| EAEC133 | ECs4910 | 9-18, 21-27, 42-49, 58-75, 85-93, 100-106, 108-122, 125-131, 140-150, 155-162, | 700 | 862 |
| | | 165-186, 201-206, 209-214, 220-249, 261-267, 279-289, 292-299, 304-310, | | |
| | | 328-338, 343-355, 370-378, 381-389, 393-400, 411-418, 424-436, 438-449, | | |
| | | 474-496, 524-531, 556-572, 586-592, 606-617, 623-629, 643-657, 659-676, | | |
| | | 696-702, 709-717, 735-755, 763-775, 788-809, 835-843 | | |
| EAEC134 | ECs4914 | 20-32, 52-63, 75-88, 96-101, 116-154, 164-173, 187-199, 202-240, 253-279, | 701 | 863 |
| | | 285-298, 305-318, 324-339 | | |
| EAEC135 | ECs5012 | 4-22, 24-29, 36-43, 63-75, 90-96, 118-128, 137-145, 168-182, 200-210, 212-221, | 702 | 864 |
| | | 242-250, 289-316, 318-323, 327-339, 381-387, 401-411, 424-434, 443-449, | | |
| | | 453-465, 485-498, 500-508, 510-515, 521-528, 538-545, 554-560, 574-606, | | |
| | | 619-627, 645-658, 681-688 | | |
| EAEC136 | ECs5026 | 8-18, 45-50, 52-62, 76-82, 84-107, 109-116, 130-137, 141-150, 152-158, | 703 | 865 |
| | | 164-170, 175-186, 188-196 | | |
| EAEC137 | ECs5061 | 11-19, 24-34, 41-48, 55-63, 68-81, 85-91, 100-106, 111-120, 131-138, 144-161, | 704 | 866 |
| | | 168-176, 192-203, 213-225, 227-234, 236-243, 255-262, 265-274, 282-290, | | |
| | | 296-301, 309-316, 349-359, 368-377, 384-390, 398-412, 417-433, 439-448, | | |
| | | 467-475, 481-486, 501-508, 510-517, 521-532, 538-545 | | |
| EAEC162 | ECs5079 | 4-42, 48-59, 74-88, 92-119, 121-149, 163-180, 185-195, 199-209 | 705 | 867 |
| EAEC138 | ECs5087 | 5-26, 60-76, 104-114, 119-128, 136-141, 156-167, 186-198, 218-237, 260-267, | 706 | 868 |
| | | 275-290, 309-318, 328-335 | | |
| EAEC139 | ECs5147 | 5-17, 23-48, 60-73, 75-82, 98-108, 110-128, 146-160, 168-180, 191-213, | 707 | 869 |
| | | 229-237, 240-252, 269-277, 305-313 | | |
| EAEC140 | ECs5191 | 8-28, 53-62, 70-78, 81-89, 97-115, 125-148, 155-168, 174-186, 196-202, | 708 | 870 |
| | | 207-214, 220-238, 241-256, 258-267, 284-290, 297-306, 312-317, 322-327, | | |
| | | 330-344, 352-358, 367-385, 387-395, 400-414, 422-430, 438-455, 458-466, | | |
| | | 491-507, 539-544, 561-566, 571-577, 598-604, 617-623, 640-647 | | |
| EAEC142 | b1368 | 7-14, 24-32, 56-72, 95-100, 108-114, 123-138, 143-153, 203-221, 224-230, | 709 | 871 |
| | | 260-269, 290-297, 302-308, 321-355, 364-370, 398-427, 434-439, 446-473, | | |
| | | 505-510, 512-518, 536-546, 573-587, 589-595, 629-636, 683-709, 728-734, | | |
| | | 778-786, 795-802, 825-830, 911-934, 944-956, 960-970, 977-985, 987-993, | | |
| | | 1009-1015, 1027-1035, 1047-1052, 1058-1065 | | |
| EAEC144 | b3480 | 4-16, 21-36, 38-47, 54-64, 92-103, 117-126, 135-155, 157-200, 202-223, | 710 | 872 |
| | | 231-239, 246-262 | | |
| EAEC146 | b3689 | 4-10, 35-45, 56-92, 108-116, 127-133, 137-146, 156-186, 194-203, 225-234, | 711 | 873 |
| | | 242-251, 272-283, 285-306, 310-315, 322-330, 358-371, 373-379, 389-396 | | |
| ARF1 | ARF0254 | 4-40, 47-59, 62-75, 81-87, 107-113 | 712 | 874 |
| ARF151 | ARF0600 | 6-13, 21-31, 44-54, 64-70, 77-84 | 713 | 875 |
| ARF2 | ARF0965 | 17-24, 26-41, 50-55 | 714 | 876 |
| ARF24 | ARF1004 | 24-32 | 715 | 877 |
| ARF3 | ARF1396 | 15-28, 36-44 | 716 | 878 |
| ARF4 | ARF1603 | 4-25, 28-34, 37-43, 45-52, 59-69, 85-91, 104-114 | 717 | 879 |
| ARF5 | ARF2885 | 13-39, 51-63, 80-101, 141-149, 165-176, 191-210 | 718 | 880 |
| ARF6 | ARF3364 | 6-19 | 719 | 881 |
| ARF7 | ARF3558 | 4-19, 23-42, 57-66, 98-114, 121-127, 148-155, 164-181, 194-204, 217-222, | 720 | 882 |
| | | 248-258, 266-285, 288-296, 309-329, 331-342, 344-353, 361-378 | | |
| ARF8 | ARF3629 | 4-14, 26-32 | 721 | 883 |
| ARF9 | ARF3673 | 10-18, 29-62, 72-91, 106-120, 147-154, 185-195, 203-210, 220-238, 262-267, | 722 | 884 |
| | | 275-285, 315-365, 376-388, 390-399, 414-423 | | |
| ARF150 | ARF4206 | 8-17, 43-49, 63-70, 79-86, 89-100 | 723 | 885 |
| ARF10 | ARF4334 | 4-19, 21-31, 54-68 | 724 | 886 |
| ARF13 | ARF4425 | 4-10, 26-31 | 725 | 887 |
| ARF14 | ARF4467 | 4-12, 21-30, 37-51 | 726 | 888 |
| ARF15 | ARF4692 | 4-11, 20-46 | 727 | 889 |
| ARF16 | ARF4739 | 4-10, 23-28 | 728 | 890 |
| ARF17 | ARF4851 | 4-28, 32-50 | 729 | 891 |
| ARF18 | ARF5002 | 4-25 | 730 | 892 |
| ARF19 | ARF5020 | 35-53 | 731 | 893 |
| ARF149 | ARF5347 | 14-20, 48-56, 63-68, 79-87 | 732 | 894 |
| ARF143 | ARF-b3374 | 34-40, 50-62 | 733 | 895 |
| ARF145 | ARF-b3577 | 23-34, 59-72 | 734 | 896 |
| CRF1 | CRF0147 | 4-16, 28-39, 62-68, 85-97 | 735 | 897 |
| CRF2 | CRF0173 | 10-20, 23-31, 35-42, 48-62 | 736 | 898 |
| CRF3 | CRF0372 | 4-44, 46-52, 54-64, 70-85 | 737 | 899 |
| CRF4 | CRF1408 | 4-12, 24-40 | 738 | 900 |
| CRF5 | CRF1476 | 10-22, 24-46, 52-57, 74-81 | 739 | 901 |
| CRF148 | CRF1683 | 10-20, 27-40, 48-57 | 740 | 902 |
| CRF6 | CRF1902 | 24-37 | 741 | 903 |
| CRF7 | CRF2012 | 20-29, 39-45, 53-65, 67-85 | 742 | 904 |
| CRF8 | CRF2570 | 6-22, 25-41, 43-66, 74-84, 87-94, 107-113 | 743 | 905 |
| CRF9 | CRF2696 | 4-12, 17-23, 39-62 | 744 | 906 |
| CRF10 | CRF2937 | 4-13, 21-45, 51-70 | 745 | 907 |
| CRF11 | CRF3041 | 15-25, 33-52 | 746 | 908 |
| CRF12 | CRF3160 | 8-19, 44-53, 60-70, 78-85, 97-107 | 747 | 909 |
| CRF14 | CRF3314 | 13-23, 31-40, 59-66, 84-90, 96-110 | 748 | 910 |
| CRF15 | CRF3625 | 4-25, 37-48, 56-71, 83-97, 112-132, 140-150, 152-157 | 749 | 911 |
| CRF16 | CRF3797 | 4-17, 19-26, 41-49, 63-87, 92-99, 113-131 | 750 | 912 |
| CRF17 | CRF3808 | 4-10, 17-23 | 751 | 913 |
| CRF18 | CRF4119 | 4-19, 26-35, 43-50, 61-72 | 752 | 914 |
| CRF19 | CRF4266 | 5-10, 31-38, 42-70, 84-99 | 753 | 915 |
| CRF20 | CRF4320 | 12-26, 48-55 | 754 | 916 |
| CRF21 | CRF4483 | 4-10, 13-63, 69-81, 83-105, 143-150 | 755 | 917 |
| CRF22 | CRF4752 | 4-12, 18-29, 49-68 | 756 | 918 |
| CRF23 | CRF4786 | 19-25, 27-33, 43-84, 86-92, 111-118, 125-136, 138-147, 158-165, 181-192 | 757 | 919 |
| CRF24 | CRF4885 | 20-29, 50-56, 63-85, 89-98, 120-128 | 758 | 920 |
| CRF25 | CRF5063 | 4-11, 41-47 | 759 | 921 |
| CRF27 | CRF5282 | 14-27, 50-62 | 760 | 922 |
| CRF35 | CRF-b2972 | 10-19, 35-41, 43-51 | 761 | 923 |
| CRF37 | CRF-b4332 | 17-40, 47-66, 70-78 | 762 | 924 |

TABLE 5
Gene distribution analysis for a selected number of antigens in various strains of the
respective bacterial species.

| | | | | Gene | Gene | Gene |
|---|---|---|---|---|---|---|
| Antigenic | Seq ID | | | distribution | distribution | distribution |
| protein | (DNA) | Source* | Homolog** | SF | EAEC | ETEC |
| SepA | 935 | S. flexneri | EAEC | 24/46 | n.d. | n.d. |
| SF2968 | 959 | S. flexneri | EAEC | 25/46 | n.d. | n.d. |
| SF2972 | 960 | S. flexneri | EAEC | 16/46 | n.d. | n.d. |
| pCP0179 | 970 | S. flexneri | EAEC | 46/46 | 7/46 | 23/46 |
| icsB | 997 | S. flexneri | n.d. | 37/46 | n.d. | n.d. |
| ipaA | 998 | S. flexneri | n.d. | 29/46 | n.d. | n.d. |
| ipaB | 999 | S. flexneri | n.d. | 31/46 | n.d. | n.d. |
| ipaC | 1000 | S. flexneri | n.d. | 26/46 | n.d. | n.d. |
| ipaD | 1001 | S. flexneri | n.d. | 32/46 | n.d. | n.d. |
| ipaH14 | 1002 | S. flexneri | n.d. | 46/46 | n.d. | n.d. |
| ipaH45 | 1003 | S. flexneri | n.d. | 46/46 | n.d. | n.d. |
| ipaH78 | 1004 | S. flexneri | n.d. | 38/46 | n.d. | n.d. |
| ipaH98 | 1005 | S. flexneri | n.d. | 45/46 | n.d. | n.d. |
| mxiH | 1006 | S. flexneri | n.d. | 38/46 | n.d. | n.d. |
| SF0267 | 1011 | S. flexneri | n.d. | 36/46 | n.d. | n.d. |
| SF0722 | 1012 | S. flexneri | n.d. | 46/46 | n.d. | n.d. |
| SF1383 | 1018 | S. flexneri | n.d. | 46/46 | n.d. | n.d. |
| SF1880 | 1021 | S. flexneri | n.d. | 46/46 | n.d. | n.d. |
| SF1889 | 1022 | S. flexneri | n.d. | 34/46 | n.d. | n.d. |
| SF1966 | 1023 | S. flexneri | n.d. | 46/46 | n.d. | n.d. |
| SF2610 | 1027 | S. flexneri | n.d. | 45/46 | n.d. | n.d. |
| pCP0254 | 1043 | S. flexneri | n.d. | 46/46 | n.d. | n.d. |
| pCP0262 | 1044 | S. flexneri | n.d. | 46/46 | n.d. | n.d. |
| virG | 1045 | S. flexneri | n.d. | 34/46 | n.d. | n.d. |
| CRF2911 | 1061 | S. flexneri | n.d. | 46/46 | n.d. | n.d. |
| b1368 | 456 | EAEC | n.d. | n.d. | 24/46 | n.d. |
| b2969 | 457 | EAEC | n.d. | n.d. | n.d. | 0/46 |
| b2986 | 458 | EAEC | n.d. | n.d. | n.d. | 45/46 |
| b3480 | 459 | EAEC | n.d. | n.d. | 44/46 | n.d. |
| b3689 | 460 | EAEC | n.d. | n.d. | 44/46 | n.d. |
| ECs0605 | 28 | ETEC | — | n.d. | n.d. | 26/46 |
| ECs0954 | 337 | EAEC | n.d. | n.d. | 45/46 | n.d. |
| ECs1392 | 41 | ETEC | EAEC | n.d. | n.d. | n.d. |
| ECs1638 | 44 | ETEC | SF708, EAEC | 27/46 | 25/46 | 30/46 |
| ECs1646 | 46 | ETEC | SF1886, | 46/46 | 44/46 | 39/46 |
| | | | EAEC | | | |
| ECs1650 | 347 | EAEC | n.d. | n.d. | 44/46 | n.d. |
| ECs2178 | 59 | ETEC | EAEC | 10/746 | 37/46 | 34/46 |
| ECs2662 | 369 | EAEC | n.d. | n.d. | 41/46 | n.d. |
| ECs3843 | 406 | EAEC | n.d. | n.d. | 7/46 | 13/46 |
| ECs5350 | 455 | ETEC | n.d. | n.d. | n.d. | 45/46 |
| EAEC11 | 161 | ETEC | — | n.d. | n.d. | 31/46 |
| EAEC12 | 162 | EAEC | SF2973 | 8/46 | 42/46 | 1/46 |
| EAEC15 | 163 | ETEC | — | n.d. | n.d. | 32/46 |
| EAEC16 | 164 | ETEC | — | n.d. | n.d. | 17/46 |
| EAEC17 | 165 | ETEC | — | n.d. | n.d. | 24/46 |
| EAEC18 | 166 | EAEC | — | n.d. | 13/46 | n.d. |
| EAEC19 | 167 | ETEC | SepA | n.d. | n.d. | 8/46 |
| EAEC20 | 168 | ETEC | — | n.d. | n.d. | 3/46 |
| EAEC21 | 169 | EAEC | — | n.d. | 15/46 | n.d. |
| EAEC23 | 170 | EAEC | — | 8/46 | 38/46 | 36/46 |
| EAEC24 | 171 | EAEC | — | n.d. | 7/46 | n.d. |
| EAEC25 | 172 | EAEC | — | 10/46 | 44/46 | 46/46 |
| EAEC27 | 173 | EAEC | — | n.d. | 15/46 | n.d. |
| EAEC29 | 174 | EAEC | — | n.d. | 14/46 | n.d. |
| EAEC40 | 609 | EAEC | ECs0218 | 6/46 | 39/46 | 36/46 |
| EAEC45 | 614 | EAEC | ECs0336 | 1/46 | 39/46 | 43/46 |
| EAEC47 | 617 | EAEC | Ecs0454 | n.d. | 17/46 | n.d. |
| EAEC48 | 618 | EAEC | Ecs0541 | n.d. | 15/46 | n.d. |
| EAEC49 | 21 | EAEC | ECs0541 | n.d. | 28/46 | n.d. |
| EAEC50 | 619 | EAEC | ECs0548 | n.d. | 16/46 | n.d. |
| EAEC62 | 631 | EAEC | ECs1643 | n.d. | 26/46 | n.d. |
| EAEC120 | 688 | EAEC | ECs4337 | n.d. | 7/46 | n.d. |
| EAEC126 | 694 | EAEC | SF3641, | 46/46 | 35/46 | 33/46 |
| | | | ECs4480 | | | |
| EAEC147 | 176 | EAEC | ECs3739, | 46/46 | 45/46 | 45/46 |
| | | | SF2911 | | | |

TABLE 6
Immunogenicity of epitopes in mice.

| | Seq ID | Seq ID | | peptide | Peptide | |
|---|---|---|---|---|---|---|
| ORF | DNA | Protein | clone injected | name | ELISA | aa (start-stop) |
| sepA | 935 | 1007 | SGGAS36 | SFOSepA.5 | + | 549-576 |
| | | | | SFOSepA.7 | + | 595-622 |
| SF2968 | 959 | 1031 | SFFAN65 | SFO2968.3 | +++++ | 592-616 |
| SF2972 | 960 | 1032 | SFLAM88 | SFO2972.1 | ++ | 9-22 |
| ECs0336 | 17 | 317 | EALAS19 | ECOA045.1 | +++ | 896-923 |
| ECs0454 | 20 | 320 | EAFBE64 | ECOA047.1 | + | 178-204 |
| | | | | ECOA047.3 | +++ | 222-248 |
| | | | | ECOA047.4 | ++ | 244-269 |
| | | | EAFBJ10 | ECOA047.5 | + | 265-296 |
| ECs0541 | 21 | 321 | EAFAA35 | ECOA049.1 | + | 180-209 |
| | | | | ECOA049.2 | ++ | 205-233 |
| ECS0548 | 22 | 322 | EAFBL63 | ECOA050.1 | +++++ | 242-270 |
| | | | | ECOA050.2 | ++++ | 266-294 |
| ECs0605 | 28 | 328 | ETLAD30 | ECO0605.1 | ++++ | 718-740 |
| ECs1643 | 45 | 345 | EAFAT63 | ECOA062.1 | ++ | 197-229 |
| | | | | ECOA062.3 | ++ | 225-256 |
| ECs2178 | 59 | 359 | ETLAR76 | ECO2178.1 | ++ | 42-61 |
| ECs4337 | 125 | 425 | EALAN11 | ECOA120.1 | + | 34-53 |
| ECs4480 | 131 | 431 | EALAV92 | ECOA126.1 | ++ | 1522-1552 |
| EAEC12 | 162 | 462 | EAFAP47 | ECOA012.1 | + | 415-440 |
| EAEC15 | 163 | 463 | ETLAU84 | ECOA015.1 | + | 32-51 |
| EAEC18 | 166 | 466 | EAFAI53 | ECOA018.1 | + | 342-369 |
| | | | | ECOA018.6 | ++++ | 457-484 |
| EAEC19 | 167 | 467 | ETFAP29 | ECOA019.2 | + | 554-582 |
| | | | | ECOA019.3 | + | 578-606 |
| | | | | ECOA019.4 | ++ | 632-659 |
| EAEC21 | 169 | 469 | EALBF79 | ECOA021.1 | +++ | 12-33 |
| | | | | ECOA021.2 | +++ | 27-48 |
| EAEC24 | 171 | 471 | EAFBN60 | ECOA024.2 | ++++ | 52-81 |
| | | | | ECOA024.3 | ++ | 77-105 |
| | | | | ECOA024.4 | ++ | 101-129 |
| EAEC25 | 172 | 472 | EAFBG06 | ECOA025.3 | + | 76-102 |
| EAEC27 | 173 | 473 | EALAX08 | ECOA027.1 | + | 48-77 |
| EAEC29 | 174 | 474 | EALAU94 | ECOA029.1 | + | 6-35 |
| b1368 | 156 | 456 | EAFAF23 | ECOA142.2 | + | 189-218 |
| | | | | ECOA142.3 | ++++ | 214-244 |
| b3480 | 159 | 459 | EALAX23 | ECOA144.1 | +++++ | 122-149 |
| b3689 | 160 | 460 | EALAV46 | ECOA146.1 | ++++ | 106-136 |
| EAEC160 | 177 | 477 | EALAR59 | ECOA160.2 | +++ | 204-224 |
| ECs0014 | 1 | 301 | EAFAA20 | ECOA032.3 | ++++ | 273-297 |
| ECs0041 | 2 | 302 | ETLAE30 | ECO0041.1 | ++++ | 70-88 |
| ECs0063 | 3 | 303 | ETLAT94 | ECO0063.1 | + | 134-147 |
| ECs0067 | 4 | 304 | EALAV72 | ECOA033.1 | ++ | 290-317 |
| | | | EALAY91 | ECOA033.2 | + | 367-388 |
| ECs0089 | 5 | 305 | EALAU75 | ECOA034.1 | +++ | 240-265 |
| ECs0152 | 6 | 306 | EALAD04 | ECOA035.1 | +++ | 796-824 |
| ECs0155 | 8 | 308 | EAFAQ23 | ECOA037.3 | +++ | 83-110 |
| | | | | ECOA037.4 | +++ | 106-133 |
| ECs0156 | 9 | 309 | EALAA33 | ECOA038.1 | +++++ | 120-148 |
| ECs0174 | 10 | 310 | EALAO18 | ECOA039.1 | +++ | 71-106 |
| ECs0314 | 12 | 312 | EALAO52 | ECOA041.1 | + | 278-305 |
| ECs0315 | 13 | 313 | EALAW09 | ECOA042.1 | ++++ | 115-137 |
| ECs0321 | 15 | 315 | ETLAF25 | ECO0321.1 | ++++ | 378-400 |
| ECs0322 | 16 | 316 | EALAE88 | ECOA044.1 | + | 111-137 |
| ECs0345 | 18 | 318 | ETLAD78 | ECO0345.1 | +++ | 3-19 |
| ECs0557 | 23 | 323 | ETLAD28 | ECO0557.1 | +++++ | 103-119 |
| ECs0600 | 26 | 326 | EAFBH15 | ECOA051.1 | ++ | 89-116 |
| | | | | ECOA051.2 | ++ | 112-139 |
| | | | | ECOA051.3 | +++++ | 135-162 |

Table 7: Peptide ELISA with Peptides Derived from Enteric Pathogens.

TABLE 7A

| Peptide | Sum | From aa | To aa | SeqID |
|---|---|---|---|---|
| ECO2330.1 | 2 | 259 | 268 | 390 |
| ECA2816.1 | 11 | 2 | 19 | 501 |
| ECAA001.1 | 18 | 83 | 114 | 488 |
| ECAA003.1 | 11 | 15 | 33 | 496 |
| ECAA004.1 | 17 | 52 | 84 | 498 |
| ECAA004.2 | 16 | 70 | 93 | 498 |
| ECAA005.1 | 22 | 28 | 54 | 502 |
| ECAA010.1 | 16 | 34 | 66 | 514 |
| ECAA149.1 | 6 | 4 | 32 | 528 |
| ECAA149.2 | 15 | 28 | 56 | 528 |
| ECAA150.1 | 14 | 21 | 47 | 513 |
| ECAA151.1 | 13 | 32 | 57 | 490 |
| ECC0451.1 | 3 | 96 | 115 | 542 |
| ECC0464.1 | 12 | 161 | 176 | 543 |
| ECC2336.1 | 13 | 58 | 89 | 557 |
| ECC2336.2 | 18 | 85 | 116 | 557 |
| ECC2336.3 | 22 | 112 | 143 | 557 |
| ECC2633.1 | 7 | 97 | 115 | 559 |
| ECC3088.1 | 19 | 84 | 106 | 565 |
| ECC3936.1 | 3 | 3 | 19 | 572 |
| ECC4317.1 | 5 | 38 | 63 | 575 |
| ECC4393.1 | 15 | 33 | 64 | 580 |
| ECC4393.2 | 15 | 60 | 91 | 580 |
| ECC4393.3 | 22 | 87 | 119 | 580 |
| ECCA008.1 | 3 | 93 | 123 | 558 |
| ECCA010.1 | 5 | 16 | 34 | 563 |
| ECCA013.1 | 1 | 61 | 92 | 593 |
| ECCA013.2 | 3 | 88 | 120 | 593 |
| ECCA014.1 | 2 | 48 | 76 | 568 |
| ECCA014.2 | 1 | 72 | 100 | 568 |
| ECCA014.3 | 0 | 96 | 124 | 568 |
| ECCA014.4 | 3 | 120 | 147 | 568 |
| ECCA015.1 | 2 | 111 | 146 | 569 |
| ECCA016.1 | 2 | 26 | 50 | 570 |
| ECCA016.2 | 8 | 46 | 71 | 570 |
| ECCA016.3 | 9 | 67 | 92 | 570 |
| ECCA018.1 | 6 | 11 | 46 | 573 |
| ECCA019.1 | 1 | 92 | 127 | 574 |
| ECCA021.1 | 5 | 46 | 78 | 582 |
| ECCA021.2 | 2 | 74 | 106 | 582 |
| ECCA021.3 | 3 | 102 | 133 | 582 |
| ECCA023.1 | 5 | 94 | 124 | 584 |
| ECCA024.1 | 3 | 2 | 29 | 585 |
| ECCA031.1 | 5 | 10 | 43 | 597 |
| ECCA034.1 | 1 | 24 | 51 | 600 |
| ECCA034.2 | 9 | 47 | 74 | 600 |
| ECCA034.3 | 0 | 70 | 98 | 600 |
| ECCA148.1 | 5 | 15 | 42 | 551 |
| ECCA148.2 | 3 | 38 | 58 | 551 |
| ECO2330.1 | 1 | 259 | 268 | 390 |
| ECCA032.1 | 20 | 66 | 94 | 598 |
| ECCT028.1 | 2 | 17 | 43 | 594 |
| ECO2882.1 | 5 | 178 | 198 | 375 |
| ECO3221.1 | 7 | 441 | 473 | 385 |
| ECO3221.2 | 5 | 469 | 501 | 385 |
| ECO3221.3 | 4 | 497 | 531 | 385 |
| ECO3264.1 | 3 | 105 | 127 | 387 |
| ECO3265.1 | 8 | 8 | 35 | 388 |
| ECO3265.2 | 4 | 36 | 63 | 388 |
| ECO3265.3 | 3 | 64 | 91 | 388 |
| ECO3378.1 | 3 | 216 | 231 | 391 |
| ECO3431.1 | 22 | 203 | 222 | 395 |
| ECO3832.1 | 9 | 180 | 210 | 404 |
| ECO3832.2 | 11 | 206 | 236 | 404 |
| ECO3832.3 | 1 | 232 | 261 | 404 |
| ECO3836.1 | 1 | 6 | 28 | 405 |
| ECO3937.1 | 1 | 224 | 238 | 408 |
| ECO4041.1 | 4 | 274 | 291 | 411 |
| ECO4051.1 | 2 | 134 | 148 | 413 |
| ECO4061.1 | 5 | 133 | 153 | 414 |
| ECO4084.1 | 0 | 258 | 284 | 416 |
| ECO4147.1 | 5 | 8 | 33 | 419 |
| ECO4147.2 | 6 | 28 | 53 | 419 |
| ECO4147.3 | 0 | 49 | 74 | 419 |
| ECO4198.1 | 2 | 103 | 127 | 420 |
| ECO4198.2 | 4 | 123 | 147 | 420 |
| ECO4198.3 | 1 | 143 | 166 | 420 |
| ECO4198.4 | 3 | 162 | 185 | 420 |
| ECO4216.1 | 1 | 179 | 206 | 421 |
| ECO4691.1 | 6 | 15 | 27 | 434 |
| ECO4725.1 | 2 | 240 | 261 | 435 |
| ECO4762.1 | 2 | 386 | 402 | 437 |
| ECO4784.1 | 15 | 57 | 74 | 438 |
| ECO5091.1 | 1 | 343 | 375 | 448 |
| ECO5091.2 | 9 | 371 | 403 | 448 |
| ECO5091.3 | 0 | 399 | 432 | 448 |
| ECO5153.1 | 3 | 369 | 390 | 450 |
| ECO5218.1 | 1 | 276 | 292 | 452 |
| ECO5276.1 | 3 | 196 | 227 | 453 |
| ECO5276.2 | 3 | 223 | 254 | 453 |
| ECO5276.3 | 0 | 250 | 280 | 453 |
| ECO5276.4 | 0 | 284 | 311 | 453 |
| ECO5276.5 | 1 | 307 | 334 | 453 |
| ECO5276.6 | 0 | 330 | 356 | 453 |
| ECO5276.7 | 1 | 352 | 378 | 453 |
| ECO5315.1 | 0 | 253 | 271 | 454 |
| ECO5350.1 | 2 | 419 | 432 | 455 |
| ECOA079.1 | 1 | 24 | 51 | 370 |
| ECO2330.1 | 3 | 259 | 268 | 390 |
| ECOA080.1 | 13 | 23 | 48 | 371 |
| ECOA081.1 | 16 | 179 | 198 | 372 |
| ECOA082.1 | 17 | 431 | 455 | 373 |
| ECOA083.1 | 14 | 319 | 349 | 374 |
| ECOA083.2 | 20 | 345 | 374 | 374 |
| ECOA083.3 | 11 | 370 | 399 | 374 |
| ECOA084.1 | 13 | 455 | 479 | 376 |
| ECOA084.2 | 3 | 475 | 498 | 376 |
| ECOA085.1 | 16 | 653 | 675 | 377 |
| ECOA086.1 | 13 | 75 | 108 | 378 |
| ECOA087.1 | 10 | 1362 | 1388 | 379 |
| ECOA088.1 | 3 | 8 | 36 | 380 |
| ECOA088.2 | 15 | 32 | 60 | 380 |
| ECOA088.3 | 19 | 56 | 84 | 380 |
| ECOA089.1 | 14 | 1 | 25 | 381 |
| ECOA090.1 | 7 | 65 | 88 | 382 |
| ECOA091.1 | 6 | 102 | 134 | 383 |
| ECOA092.1 | 7 | 104 | 131 | 384 |
| ECOA093.1 | 2 | 206 | 231 | 386 |
| ECOA094.1 | 4 | 5 | 23 | 389 |
| ECOA095.1 | 1 | 510 | 536 | 390 |
| ECOA095.2 | 3 | 546 | 577 | 390 |
| ECOA096.1 | 4 | 300 | 326 | 392 |
| ECOA096.2 | 1 | 1320 | 1353 | 392 |
| ECOA097.1 | 9 | 205 | 240 | 393 |
| ECOA098.1 | 1 | 971 | 1003 | 394 |
| ECOA099.1 | 2 | 455 | 483 | 396 |
| ECOA099.2 | 18 | 1232 | 1263 | 396 |
| ECOA100.1 | 0 | 12 | 40 | 397 |
| ECOA101.1 | 2 | 3 | 32 | 398 |
| ECOA102.1 | 2 | 68 | 103 | 399 |
| ECOA103.1 | 1 | 188 | 222 | 400 |
| ECOA104.1 | 11 | 25 | 55 | 401 |
| ECOA105.1 | 3 | 98 | 130 | 402 |
| ECOA106.1 | 19 | 211 | 236 | 403 |
| ECOA107.1 | 1 | 362 | 392 | 406 |
| ECOA108.1 | 2 | 365 | 389 | 407 |
| ECOA109.1 | 2 | 60 | 81 | 409 |
| ECOA110.1 | 5 | 69 | 104 | 410 |
| ECOA111.1 | 3 | 124 | 152 | 412 |
| ECOA111.2 | 1 | 148 | 177 | 412 |
| ECOA112.1 | 3 | 74 | 102 | 415 |
| ECOA113.1 | 5 | 1019 | 1051 | 417 |
| ECOA113.2 | 0 | 1162 | 1190 | 417 |
| ECOA113.3 | 1 | 1186 | 1214 | 417 |
| ECOA113.4 | 1 | 1210 | 1238 | 417 |
| ECOA113.5 | 3 | 1234 | 1261 | 417 |
| ECOA118.1 | 11 | 692 | 724 | 423 |
| ECO2330.1 | 2 | 259 | 268 | 390 |
| ECOA116.1 | 13 | 15 | 41 | 418 |
| ECOA116.2 | 9 | 37 | 62 | 418 |
| ECOA116.3 | 16 | 58 | 83 | 418 |
| ECOA117.1 | 10 | 59 | 87 | 422 |
| ECOA117.2 | 6 | 83 | 110 | 422 |
| ECOA117.3 | 14 | 111 | 140 | 422 |
| ECOA119.1 | 15 | 174 | 206 | 424 |
| ECOA121.1 | 4 | 96 | 119 | 426 |
| ECOA122.1 | 7 | 128 | 152 | 427 |
| ECOA123.1 | 6 | 104 | 130 | 428 |
| ECOA124.1 | 10 | 542 | 568 | 429 |
| ECOA125.1 | 10 | 107 | 129 | 430 |
| ECOA127.1 | 11 | 112 | 137 | 432 |
| ECOA129.1 | 16 | 52 | 80 | 433 |
| ECOA130.1 | 6 | 19 | 45 | 436 |
| ECOA131.1 | 0 | 1113 | 1144 | 441 |
| ECOA133.1 | 7 | 94 | 119 | 439 |
| ECOA133.2 | 0 | 110 | 135 | 439 |
| ECOA133.3 | 2 | 136 | 160 | 439 |
| ECOA134.1 | 0 | 89 | 115 | 442 |
| ECOA135.1 | 3 | 474 | 496 | 443 |
| ECOA135.2 | 1 | 492 | 515 | 443 |
| ECOA136.1 | 3 | 14 | 40 | 444 |
| ECOA137.1 | 0 | 422 | 448 | 445 |
| ECOA138.1 | 1 | 294 | 333 | 447 |
| ECOA139.1 | 6 | 113 | 140 | 449 |
| ECOA140.1 | 0 | 8 | 36 | 451 |
| ECOA140.2 | 1 | 32 | 61 | 451 |
| ECOA140.3 | 0 | 57 | 86 | 451 |
| ECOA161.1 | 1 | 1 | 28 | 440 |
| ECOA161.2 | 2 | 120 | 147 | 440 |
| ECOA161.3 | 0 | 143 | 170 | 440 |
| ECOA162.1 | 4 | −14 | 11 | 446 |

TABLE 7B

| Peptide | Sum | From aa | To aa | SeqID |
|---|---|---|---|---|
| SFO0179.1 | 20 | 96 | 124 | 1042 |
| SF2968.1 | 16 | 550 | 575 | 1031 |
| SF2968.2 | 18 | 571 | 596 | 1031 |
| SF2968.3 | 17 | 592 | 616 | 1031 |
| SFA0504.1 | 19 | 144 | 170 | 1046 |
| SFA1280.1 | 2 | 79 | 100 | 1048 |
| SFA3121.1 | 20 | 73 | 96 | 1050 |
| SFA3760.1 | 20 | 21 | 43 | 1051 |
| SFA4075.1 | 17 | 18 | 42 | 1052 |
| SFA4262.1 | 21 | 16 | 50 | 1053 |
| SFC0109.1 | 13 | 7 | 25 | 1055 |
| SFC0548.1 | 19 | 8 | 44 | 1056 |
| SFC0598.1 | 22 | 22 | 51 | 1057 |
| SFC2403.1 | 17 | 2 | 25 | 1059 |
| SFC2748.1 | 17 | 9 | 44 | 1060 |
| SFC2911.1 | 6 | 6 | 34 | 1061 |
| SFC2911.2 | 20 | 30 | 58 | 1061 |
| SFC2911.3 | 12 | 54 | 82 | 1061 |
| SFC2911.4 | 9 | 78 | 105 | 1061 |
| SFC3470.1 | 12 | 9 | 32 | 1062 |
| SFC3528.1 | 11 | 23 | 46 | 1063 |
| SFC3528.2 | 6 | 42 | 65 | 1063 |
| SFC3798.1 | 9 | 1 | 25 | 1064 |
| SFC3843.1 | 20 | 17 | 43 | 1065 |
| SFC4125.1 | 0 | 11 | 38 | 1067 |
| SFO0132.1 | 11 | 44 | 72 | 1009 |
| SFO0132.2 | 1 | 68 | 95 | 1009 |
| SFO0132.3 | 15 | 91 | 118 | 1009 |
| SFO0254.1 | 8 | 163 | 187 | 1043 |
| SFO0267.1 | 7 | 586 | 612 | 1011 |
| SFO0722.1 | 17 | 259 | 284 | 1012 |
| SFO0722.2 | 2 | 283 | 313 | 1012 |
| SFO0722.3 | 3 | 309 | 339 | 1012 |
| SFO0722.4 | 1 | 335 | 365 | 1012 |
| SFO0986.1 | 8 | 311 | 333 | 1016 |
| SFO1383.1 | 16 | 245 | 269 | 1018 |
| SFO1383.2 | 1 | 474 | 504 | 1018 |
| SFO1383.3 | 4 | 500 | 530 | 1018 |
| SFO1383.4 | 1 | 526 | 555 | 1018 |
| SFO1880.1 | 0 | 270 | 302 | 1021 |
| SFO1880.2 | 0 | 298 | 330 | 1021 |
| SFO1880.3 | 3 | 326 | 358 | 1021 |
| SFO1966.1 | 0 | 58 | 86 | 1023 |
| SFO1966.2 | 0 | 82 | 109 | 1023 |
| SFO1966.3 | 0 | 105 | 133 | 1023 |
| SFO1966.4 | 1 | 325 | 353 | 1023 |
| SFO1966.5 | 5 | 349 | 378 | 1023 |
| SFO1966.6 | 0 | 374 | 403 | 1023 |
| SFO2972.1 | 0 | 9 | 33 | 1032 |
| SFO0179.1 | 21 | 96 | 124 | 1042 |
| SFO1889.1 | 21 | 447 | 475 | 1022 |
| SFO1889.2 | 5 | 471 | 498 | 1022 |
| SFO1889.3 | 14 | 494 | 521 | 1022 |
| SFO2610.1 | 16 | 285 | 316 | 1027 |
| SFO2610.2 | 10 | 312 | 343 | 1027 |
| SFO2610.3 | 18 | 339 | 371 | 1027 |
| SFO2610.4 | 3 | 367 | 399 | 1027 |
| SFO2610.5 | 12 | 515 | 541 | 1027 |
| SFO2610.6 | 3 | 537 | 564 | 1027 |
| SFO2610.7 | 4 | 560 | 586 | 1027 |
| SFOIcsB.1 | 18 | 149 | 175 | 997 |
| SFOIcsB.2 | 3 | 171 | 197 | 997 |
| SFOIcsB.3 | 8 | 193 | 217 | 997 |
| SFOIpaA.1 | 9 | 43 | 73 | 998 |
| SFOIpaA.2 | 4 | 69 | 99 | 998 |
| SFOIpaA.3 | 4 | 95 | 124 | 998 |
| SFOIpaA.4 | 12 | 163 | 189 | 998 |
| SFOIpaA.5 | 4 | 185 | 210 | 998 |
| SFOIpaA.6 | 0 | 206 | 231 | 998 |
| SFOIpaA.7 | 14 | 227 | 252 | 998 |
| SFOIpaA.8 | 7 | 248 | 273 | 998 |
| SFOIpaA.9 | 17 | 269 | 294 | 998 |
| SFOIpaA.10 | 13 | 313 | 340 | 998 |
| SFOIpaA.11 | 7 | 336 | 362 | 998 |
| SFOIpaA.12 | 8 | 358 | 384 | 998 |
| SFOIpaA.13 | 1 | 456 | 481 | 998 |
| SFOIpaA.14 | 0 | 477 | 502 | 998 |
| SFOIpaA.15 | 4 | 498 | 523 | 998 |
| SFOIpaB.1 | 17 | 136 | 163 | 999 |
| SFOIpaB.2 | 18 | 159 | 186 | 999 |
| SFOIcsB.3 | 8 | 193 | 217 | 999 |
| SFOIpaB.4 | 0 | 253 | 279 | 999 |
| SFOIpaB.5 | 0 | 275 | 301 | 999 |
| SFOIpaB.6 | 10 | 447 | 474 | 999 |
| SFOIpaB.7 | 18 | 470 | 496 | 999 |
| SFOIpaB.8 | 6 | 492 | 518 | 999 |
| SFOIpaB.9 | 16 | 519 | 547 | 999 |
| SFOIpaB.10 | 3 | 543 | 572 | 999 |
| SFOIpaC.1 | 17 | 31 | 58 | 1000 |
| SFOIpaC.2 | 5 | 54 | 80 | 1000 |
| SFOIpaC.3 | 19 | 76 | 102 | 1000 |
| SFOIpaC.4 | 1 | 166 | 196 | 1000 |
| SFOIpaC.5 | 9 | 222 | 246 | 1000 |
| SFOIpaC.6 | 17 | 242 | 266 | 1000 |
| SFOIpaC.7 | 12 | 284 | 310 | 1000 |
| SFOIpaC.8 | 18 | 306 | 332 | 1000 |
| SFOIpaC.9 | 2 | 328 | 354 | 1000 |
| SFO0179.1 | 13 | 96 | 124 | 1042 |
| SFOIpaD.1 | 7 | 35 | 58 | 1001 |
| SFOIpaD.2 | 8 | 54 | 77 | 1001 |
| SFOIpaD.3 | 8 | 73 | 96 | 1001 |
| SFOIpaH14.1 | 4 | 200 | 229 | 1002 |
| SFOIpaH14.2 | 13 | 225 | 253 | 1002 |
| SFOIpaH14.3 | 20 | 249 | 277 | 1002 |
| SFOIpaH14.4 | 10 | 495 | 522 | 1002 |
| SFOIpaH14.5 | 7 | 518 | 544 | 1002 |
| SFOIpaH14.6 | 6 | 540 | 566 | 1002 |
| SFOIpaH45.1 | 11 | 238 | 267 | 1003 |
| SFOIpaH45.2 | 18 | 268 | 300 | 1003 |
| SFOIpaH45.3 | 11 | 301 | 330 | 1003 |
| SFOIpaH78.1 | 11 | 6 | 36 | 1004 |
| SFOIpaH78.2 | 8 | 32 | 62 | 1004 |
| SFOIpaH78.3 | 7 | 58 | 87 | 1004 |
| SFOIpaH78.4 | 7 | 210 | 237 | 1004 |
| SFOIpaH78.5 | 16 | 233 | 260 | 1004 |
| SFOIpaH78.6 | 1 | 256 | 282 | 1004 |
| SFOIpaH78.7 | 1 | 496 | 522 | 1004 |
| SFOIpaH78.8 | 6 | 518 | 543 | 1004 |
| SFOIpaH78.9 | 0 | 539 | 564 | 1004 |
| SFOIpaH98.1 | 5 | 26 | 53 | 1005 |
| SFOIpaH98.10 | 4 | 468 | 501 | 1005 |
| SFOIpaH98.11 | 9 | 497 | 530 | 1005 |
| SFOIpaH98.2 | 4 | 49 | 76 | 1005 |
| SFOIpaH98.3 | 6 | 72 | 98 | 1005 |
| SFOIpaH98.4 | 14 | 235 | 268 | 1005 |
| SFOIpaH98.5 | 7 | 253 | 283 | 1005 |
| SFOIpaH98.6 | 4 | 279 | 309 | 1005 |
| SFOIpaH98.7 | 0 | 305 | 334 | 1005 |
| SFOIpaH98.8 | 3 | 425 | 455 | 1005 |
| SFOIpaH98.9 | 2 | 439 | 472 | 1005 |
| SFOMxiH.1 | 0 | 7 | 40 | 1006 |
| SFOMxiH.2 | 0 | 36 | 69 | 1006 |
| SFOVirG.1 | 2 | 11 | 38 | 1045 |
| SFOVirG.2 | 3 | 34 | 61 | 1045 |
| SFOVirG.3 | 13 | 57 | 84 | 1045 |
| SFOVirG.4 | 11 | 126 | 153 | 1045 |
| SFOVirG.5 | 3 | 149 | 176 | 1045 |
| SFOVirG.6 | 1 | 172 | 198 | 1045 |
| SFOVirG.7 | 10 | 265 | 290 | 1045 |
| SFOVirG.8 | 0 | 286 | 311 | 1045 |
| SFOVirG.9 | 0 | 307 | 331 | 1045 |

TABLE 7C

| Peptide | Sum | From aa | To aa | SeqID |
|---|---|---|---|---|
| ECOA042.1 | 1 | 115 | 137 | 313 |
| CJA0021.1 | 20 | 11 | 35 | 1307 |
| CJA0791.1 | 14 | 1 | 25 | 1312 |
| CJA1045.1 | 17 | 8 | 32 | 1313 |
| CJA1410.1 | 15 | 5 | 29 | 1317 |
| CJA1454.1 | 13 | 10 | 34 | 1318 |
| CJC0036.1 | 12 | 82 | 106 | 1320 |
| CJC0037.1 | 10 | 3 | 27 | 1321 |
| CJC0174.1 | 20 | 9 | 33 | 1322 |
| CJC0337.1 | 10 | 1 | 25 | 1323 |
| CJC0412.1 | 17 | 16 | 40 | 1324 |
| CJC0442.1 | 6 | 4 | 35 | 1325 |
| CJC0442.2 | 4 | 31 | 61 | 1325 |
| CJC0631.1 | 14 | 67 | 91 | 1326 |
| CJC0999.1 | 7 | 25 | 52 | 1327 |
| CJC1059.1 | 4 | 20 | 44 | 1329 |
| CJC1123.1 | 8 | 1 | 25 | 1330 |
| CJC1246.1 | 0 | 5 | 32 | 1331 |
| CJC1246.2 | 3 | 28 | 54 | 1331 |
| CJC1257.1 | 3 | 14 | 38 | 1332 |
| CJC1287.1 | 3 | 23 | 47 | 1333 |
| CJC1502.1 | 7 | 8 | 32 | 1334 |
| CJC1567.1 | 1 | 18 | 42 | 1335 |
| CJC1582.1 | 8 | 3 | 27 | 1336 |
| CJO0005.1 | 5 | 181 | 203 | 1203 |
| CJO0007.1 | 2 | 450 | 474 | 1204 |
| CJO0029.1 | 4 | 200 | 224 | 1205 |
| CJO0029.2 | 1 | 220 | 245 | 1205 |
| CJO0029.3 | 0 | 241 | 266 | 1205 |
| CJO0037.1 | 0 | 50 | 79 | 1206 |
| CJO0037.2 | 1 | 75 | 104 | 1206 |
| CJO0079.1 | 4 | 195 | 219 | 1207 |
| CJO0093.1 | 2 | 26 | 53 | 1208 |
| CJO0093.2 | 0 | 49 | 77 | 1208 |
| CJO0113.1 | 1 | 107 | 136 | 1209 |
| CJO0113.2 | 2 | 132 | 162 | 1209 |
| CJO0134.1 | 1 | 197 | 221 | 1210 |
| CJO0143.1 | 0 | 10 | 34 | 1211 |
| CJO0158.1 | 0 | 48 | 80 | 1212 |
| CJO0158.2 | 0 | 76 | 107 | 1212 |
| CJO0178.1 | 0 | 159 | 191 | 1213 |
| CJO0178.2 | 0 | 187 | 218 | 1213 |
| CJO0178.3 | 0 | 434 | 464 | 1213 |
| CJO0178.4 | 0 | 460 | 489 | 1213 |
| CJO0181.1 | 0 | 66 | 90 | 1214 |
| CJO0264.1 | 0 | 51 | 75 | 1215 |
| CJO0264.2 | 1 | 666 | 690 | 1215 |
| CJO0281.1 | 1 | 99 | 123 | 1216 |
| CJO0285.1 | 2 | 251 | 275 | 1217 |
| ECOA042.1 | 5 | 115 | 137 | 313 |
| CJO0297.1 | 8 | 102 | 129 | 1218 |
| CJO0297.2 | 5 | 125 | 151 | 1218 |
| CJO0396.1 | 13 | 30 | 54 | 1219 |
| CJO0406.1 | 4 | 138 | 162 | 1220 |
| CJO0415.1 | 5 | 79 | 103 | 1221 |
| CJO0432.1 | 7 | 101 | 125 | 1222 |
| CJO0448.1 | 3 | 126 | 154 | 1223 |
| CJO0448.2 | 0 | 150 | 178 | 1223 |
| CJO0448.3 | 2 | 174 | 202 | 223 |
| C100505.1 | 6 | 322 | 346 | 1224 |
| CJO0543.1 | 6 | 418 | 442 | 1225 |
| CJO0548.1 | 5 | 126 | 156 | 1226 |
| CJO0548.2 | 1 | 152 | 182 | 1226 |
| CJO0548.3 | 4 | 178 | 208 | 1226 |
| CJO0548.4 | 1 | 436 | 465 | 1226 |
| C100548.5 | 0 | 461 | 489 | 1226 |
| CJO0548.6 | 0 | 485 | 513 | 1226 |
| CJO0596.1 | 0 | 42 | 75 | 1227 |
| CJO0596.2 | 3 | 71 | 104 | 1227 |
| CJO0596.3 | 0 | 134 | 161 | 1227 |
| CJO0596.4 | 0 | 157 | 184 | 1227 |
| CJO0596.5 | 0 | 181 | 209 | 1227 |
| CJO0596.6 | 1 | 205 | 233 | 1227 |
| CJO0599.1 | 1 | 131 | 155 | 1228 |
| CJO0599.2 | 0 | 262 | 286 | 1228 |
| CJO0613.1 | 3 | 43 | 69 | 1229 |
| CJO0613.2 | 0 | 65 | 90 | 1229 |
| CJO0631.1 | 2 | 468 | 492 | 1230 |
| CJO0642.1 | 12 | 90 | 115 | 1231 |
| CJO0642.2 | 0 | 111 | 135 | 1231 |
| CJO0642.3 | 0 | 131 | 155 | 1231 |
| CJO0642.4 | 0 | 151 | 175 | 1231 |
| CJO0652.1 | 0 | 495 | 522 | 1232 |
| CJO0652.2 | 2 | 518 | 545 | 1232 |
| CJO0683.1 | 0 | 45 | 72 | 1233 |
| CJO0683.2 | 0 | 68 | 95 | 1233 |
| CJO0687.1 | 2 | 94 | 121 | 1234 |
| CJO0687.2 | 0 | 117 | 144 | 1234 |
| CJO0689.1 | 2 | 8 | 32 | 1235 |
| CJO0689.2 | 0 | 337 | 361 | 1235 |
| CJO0690.1 | 0 | 943 | 973 | 1236 |
| CJO0690.2 | 0 | 969 | 1000 | 1236 |
| CJO0692.1 | 0 | 191 | 215 | 1237 |
| CJO0696.1 | 0 | 70 | 97 | 1238 |
| CJO0696.2 | 0 | 93 | 120 | 1238 |
| CJO0718.1 | 1 | 800 | 824 | 1242 |
| CJO0718.2 | 1 | 820 | 844 | 1242 |
| CJO0718.3 | 2 | 840 | 864 | 1242 |
| ECOA042.1 | 4 | 115 | 137 | 313 |
| CJO0709.1 | 3 | 341 | 363 | 1241 |
| CJO0709.2 | 1 | 359 | 381 | 1241 |
| CJO0709.3 | 9 | 376 | 402 | 1241 |
| CJO0709.4 | 13 | 398 | 424 | 1241 |
| CJO0709.5 | 9 | 420 | 445 | 1241 |
| CJO0720.1 | 7 | 186 | 216 | 1243 |
| CJO0723.1 | 10 | 103 | 127 | 1244 |
| CJO0737.1 | 6 | 19 | 50 | 1245 |
| CJO0737.2 | 3 | 46 | 77 | 1245 |
| CJO0753.1 | 19 | 80 | 107 | 1246 |
| CJO0753.2 | 4 | 103 | 129 | 1246 |
| CJO0753.3 | 1 | 125 | 151 | 1246 |
| CJO0755.1 | 2 | 406 | 430 | 1247 |
| CJO0775.1 | 2 | 795 | 827 | 1248 |
| CJO0775.2 | 3 | 823 | 855 | 1248 |
| CJO0802.1 | 3 | 404 | 428 | 1249 |
| CJO0814.1 | 3 | 66 | 90 | 1250 |
| CJO0814.2 | 1 | 86 | 110 | 1250 |
| CJO0814.3 | 0 | 106 | 130 | 1250 |
| CJO0832 | 17 | 107 | 131 | 1251 |
| CJO0849 | 4 | 524 | 548 | 1252 |
| CJO0887.1 | 3 | 193 | 217 | 1253 |
| CJO0887.2 | 1 | 293 | 323 | 1253 |
| CJO0887.3 | 0 | 319 | 349 | 1253 |
| CJO0887.4 | 0 | 345 | 375 | 1253 |
| CJO0887.5 | 0 | 371 | 400 | 1253 |
| CJO0942.1 | 2 | 481 | 505 | 1254 |
| CJO0958.1 | 7 | 33 | 58 | 1255 |
| CJO0958.2 | 0 | 54 | 78 | 1255 |
| CJO1013.1 | 1 | 281 | 308 | 1256 |
| CJO1013.2 | 3 | 304 | 331 | 1256 |
| CJO1013.3 | 1 | 327 | 354 | 1256 |
| CJO1031.1 | 2 | 321 | 352 | 1257 |
| CJO1032.1 | 2 | 174 | 198 | 1258 |
| CJO1033.1 | 0 | 248 | 273 | 1259 |
| CJO1033.2 | 3 | 269 | 293 | 1259 |
| CJO1033.3 | 0 | 289 | 313 | 1259 |
| CJO1048.1 | 3 | 249 | 281 | 1260 |
| CJO1048.2 | 4 | 277 | 310 | 1260 |
| CJO1061.1 | 2 | 31 | 55 | 1261 |
| CJO1073.1 | 2 | 415 | 439 | 1262 |
| CJO1073.2 | 8 | 540 | 566 | 1262 |
| CJO1073.3 | 8 | 562 | 589 | 1262 |
| CJO1076.1 | 0 | 213 | 237 | 1263 |
| CJO1126.1 | 1 | 243 | 267 | 1264 |
| CJO1155.1 | 1 | 545 | 583 | 1265 |
| CJO1229.1 | 0 | 73 | 97 | 1267 |
| CJO1229.2 | 3 | 240 | 264 | 1267 |
| ECOA042.1 | 2 | 115 | 137 | 313 |
| CJO1184.1 | 8 | 48 | 73 | 1266 |
| CJO1184.2 | 10 | 69 | 93 | 1266 |
| CJO1184.3 | 10 | 89 | 113 | 1266 |
| CJO1236.1 | 12 | 9 | 33 | 1269 |
| CJO1262.1 | 9 | 375 | 399 | 1270 |
| CJO1266.1 | 13 | 335 | 359 | 1271 |
| CJO1266.2 | 12 | 461 | 489 | 1271 |
| CJO1266.3 | 10 | 485 | 513 | 1271 |
| CJO1266.4 | 11 | 509 | 536 | 1271 |
| CJO1267.1 | 13 | 30 | 62 | 1272 |
| CJO1267.2 | 11 | 58 | 90 | 1272 |
| CJO1272.1 | 14 | 581 | 605 | 1273 |
| CJO1290.1 | 13 | 101 | 131 | 1274 |
| CJO1290.2 | 10 | 127 | 157 | 1274 |
| CJO1316.1 | 7 | 317 | 349 | 1275 |
| CJO1316.2 | 8 | 345 | 377 | 1275 |
| CJO1333.1 | 9 | 534 | 558 | 1276 |
| CJO1338.1 | 6 | 108 | 133 | 1277 |
| CJO1338.2 | 8 | 234 | 261 | 1277 |
| CJO1338.3 | 7 | 257 | 284 | 1277 |
| CJO1338.4 | 4 | 280 | 307 | 1277 |
| CJO1338.5 | 6 | 303 | 330 | 1277 |
| CJO1339.1 | 7 | 2 | 27 | 1278 |
| CJO1339.2 | 6 | 23 | 48 | 1278 |
| CJO1339.3 | 5 | 44 | 68 | 1278 |
| CJO1339.4 | 0 | 82 | 106 | 1278 |
| CJO1339.5 | 0 | 104 | 128 | 1278 |
| CJO1339.6 | 1 | 152 | 176 | 1278 |
| CJO1339.7 | 0 | 373 | 397 | 1278 |
| CJO1341.1 | 0 | 324 | 348 | 1279 |
| CJO1342.1 | 0 | 196 | 223 | 1280 |
| CJO1342.2 | 0 | 219 | 246 | 1280 |
| CJO1346.1 | 0 | 263 | 294 | 1281 |
| CJO1346.2 | 0 | 291 | 323 | 1281 |
| CJO1379.1 | 3 | 183 | 207 | 1282 |
| CJO1380.1 | 0 | 176 | 200 | 1283 |
| CJO1422.1 | 0 | 297 | 321 | 1284 |
| CJO1426.1 | 0 | 91 | 115 | 1285 |
| CJO1463.1 | 0 | 11 | 41 | 1286 |
| CJO1463.2 | 0 | 37 | 67 | 1286 |
| CJO1463.3 | 0 | 63 | 92 | 1286 |
| CJO1466.1 | 0 | 157 | 190 | 1287 |
| CJO1466.2 | 0 | 186 | 219 | 1287 |
| CJO1474.1 | 0 | 240 | 264 | 1288 |
| CJO1478.1 | 2 | 51 | 75 | 1289 |
| CJO1484.1 | 1 | 31 | 55 | 1290 |
| CJO1500.1 | 4 | 166 | 190 | 1291 |
| CJO1511.1 | 2 | 10 | 34 | 1293 |
| ECOA042.1 | 11 | 115 | 137 | 313 |
| CJO1506.1 | 5 | 554 | 579 | 1292 |
| CJO1506.2 | 8 | 575 | 600 | 1292 |
| CJO1506.3 | 6 | 596 | 621 | 1292 |
| CJO1506.4 | 6 | 617 | 642 | 1292 |
| CJO1552.1 | 8 | 200 | 226 | 1295 |
| CJO1552.2 | 20 | 222 | 248 | 1295 |
| CJO1552.3 | 17 | 244 | 269 | 1295 |
| CJO1553.1 | 7 | 4 | 28 | 1296 |
| CJO1564.1 | 6 | 476 | 503 | 1297 |
| CJO1584.1 | 8 | 370 | 394 | 1298 |
| CJO1609.1 | 15 | 129 | 153 | 1299 |
| CJO1634.1 | 7 | 51 | 75 | 1301 |
| CJO1670.1 | 3 | 88 | 112 | 1302 |
| CJO1678.1 | 4 | 670 | 694 | 1303 |
| CJO1678.2 | 5 | 848 | 872 | 1303 |
| CJO1729.1 | 8 | 164 | 196 | 1306 |
| CJO1729.10 | 1 | 703 | 731 | 1306 |
| CJO1729.11 | 1 | 764 | 796 | 1306 |
| CJO1729.12 | 2 | 792 | 823 | 1306 |
| CJO1729.2 | 2 | 192 | 223 | 1306 |
| CJO1729.3 | 2 | 219 | 250 | 1306 |
| CJO1729.4 | 2 | 438 | 463 | 1306 |
| CJO1729.5 | 2 | 459 | 483 | 1306 |
| CJO1729.6 | 2 | 479 | 503 | 1306 |
| CJO1729.7 | 1 | 629 | 658 | 1306 |
| CJO1729.8 | 0 | 654 | 683 | 1306 |
| CJO1729.9 | 0 | 679 | 707 | 1306 |

TABLE 7D

| Peptide | Sum | From aa | To aa | SeqID |
|---|---|---|---|---|
| ECOA011.1 | 22 | 5 | 36 | 461 |
| ECOA015.1 | 21 | 32 | 51 | 463 |
| ECOA020.1 | 13 | 16 | 46 | 468 |
| ECOA021.1 | 10 | 12 | 33 | 469 |
| ECOA021.2 | 9 | 27 | 48 | 469 |
| ECOA023.1 | 18 | 10 | 39 | 470 |
| ECOA024.1 | 18 | 27 | 56 | 471 |
| ECOA024.2 | 19 | 52 | 81 | 471 |
| ECOA024.3 | 9 | 77 | 105 | 471 |
| ECOA024.4 | 9 | 101 | 129 | 471 |
| ECOA025.1 | 30 | 30 | 57 | 472 |
| ECOA025.2 | 41 | 53 | 80 | 472 |
| ECOA025.3 | 27 | 76 | 102 | 472 |
| ECOA027.1 | 7 | 48 | 77 | 473 |
| ECOA029.1 | 9 | 6 | 35 | 474 |
| ECOA032.1 | 13 | 233 | 257 | 301 |
| ECOA032.2 | 1 | 253 | 277 | 301 |
| ECOA032.3 | 3 | 273 | 297 | 301 |
| ECOA032.4 | 9 | 293 | 317 | 301 |
| ECOA033.1 | 7 | 290 | 317 | 303 |
| ECOA033.2 | 9 | 367 | 388 | 303 |
| ECOA034.1 | 1 | 240 | 265 | 305 |
| ECOA035.1 | 30 | 796 | 824 | 306 |
| ECOA036.1 | 4 | 624 | 653 | 307 |
| ECOA037.1 | 17 | 36 | 64 | 308 |
| ECOA037.2 | 5 | 60 | 87 | 308 |
| ECOA037.3 | 1 | 83 | 110 | 308 |
| ECOA037.4 | 1 | 106 | 133 | 308 |
| ECOA038.1 | 13 | 120 | 148 | 309 |
| ECOA039.1 | 9 | 71 | 106 | 310 |
| ECOA040.1 | 6 | 741 | 764 | 311 |
| ECOA040.2 | 2 | 760 | 783 | 311 |
| ECOA043.1 | 2 | 450 | 483 | 314 |
| ECOA044.1 | 2 | 111 | 137 | 316 |
| ECOA045.1 | 3 | 896 | 923 | 317 |
| ECOA046.1 | 11 | 565 | 595 | 319 |
| ECOA047.1 | 1 | 178 | 204 | 320 |
| ECOA047.2 | 2 | 200 | 226 | 320 |
| ECOA047.3 | 2 | 222 | 248 | 320 |
| ECOA047.4 | 2 | 244 | 269 | 320 |
| ECOA047.5 | 5 | 265 | 296 | 320 |
| ECOA048.4 | 7 | 1343 | 1369 | 321 |
| ECOA049.1 | 5 | 180 | 209 | 321 |
| ECOA049.2 | 3 | 205 | 233 | 321 |
| ECOA049.3 | 1 | 229 | 258 | 321 |
| ECOA050.1 | 2 | 242 | 270 | 322 |
| ECOA050.2 | 2 | 266 | 294 | 322 |
| ECOA050.3 | 2 | 290 | 318 | 322 |
| ECO2330.1 | 4 | 259 | 268 | 360 |
| ECOA041.1 | 13 | 278 | 305 | 312 |
| ECOA042.1 | 35 | 115 | 137 | 313 |
| ECOA051.1 | 17 | 89 | 116 | 326 |
| ECOA051.2 | 16 | 112 | 139 | 326 |
| ECOA051.3 | 15 | 135 | 162 | 326 |
| ECOA052.1 | 21 | 76 | 97 | 329 |
| ECOA052.2 | 30 | 93 | 114 | 329 |
| ECOA053.1 | 17 | 39 | 66 | 331 |
| ECOA053.2 | 23 | 62 | 88 | 331 |
| ECOA053.3 | 32 | 84 | 110 | 331 |
| ECOA054.1 | 29 | 244 | 274 | 333 |
| ECOA055.1 | 15 | 548 | 572 | 335 |
| ECOA056.1 | 31 | 165 | 200 | 336 |
| ECOA058.1 | 15 | 1 | 35 | 338 |
| ECOA059.1 | 18 | 662 | 695 | 339 |
| ECOA060.1 | 23 | 232 | 256 | 342 |
| ECOA060.2 | 6 | 252 | 283 | 342 |
| ECOA061.1 | 9 | 588 | 620 | 343 |
| ECOA062.1 | 10 | 197 | 229 | 345 |
| ECOA062.2 | 10 | 225 | 256 | 345 |
| ECOA062.3 | 9 | 252 | 283 | 345 |
| ECOA063.1 | 11 | 212 | 244 | 347 |
| ECOA063.2 | 5 | 240 | 272 | 347 |
| ECOA063.3 | 7 | 395 | 429 | 347 |
| ECOA064.1 | 27 | 209 | 230 | 348 |
| ECOA065.1 | 36 | 78 | 106 | 349 |
| ECOA065.2 | 14 | 102 | 130 | 349 |
| ECOA066.1 | 8 | 118 | 142 | 350 |
| ECOA067.1 | 5 | 105 | 133 | 351 |
| ECOA067.2 | 9 | 129 | 156 | 351 |
| ECOA067.3 | 9 | 152 | 180 | 351 |
| ECOA068.1 | 2 | 147 | 175 | 352 |
| ECOA069.1 | 7 | 16 | 44 | 353 |
| ECOA070.1 | 4 | 102 | 131 | 354 |
| ECOA071.1 | 7 | 328 | 355 | 355 |
| ECOA072.1 | 7 | 436 | 465 | 357 |
| ECOA074.1 | 23 | 139 | 166 | 363 |
| ECOA076.1 | 8 | 373 | 401 | 365 |
| ECOA077.1 | 6 | 114 | 148 | 367 |
| ECOA078.1 | 6 | 23 | 48 | 369 |
| ECOA078.2 | 3 | 44 | 70 | 369 |
| ECOA078.3 | 0 | 71 | 99 | 369 |
| ECOA120.1 | 2 | 34 | 53 | 425 |
| ECOA142.1 | 2 | 164 | 193 | 456 |
| ECOA142.2 | 4 | 189 | 218 | 456 |
| ECOA142.3 | 10 | 214 | 244 | 456 |
| ECOA126.1 | 15 | 1522 | 1552 | 431 |
| ECOA141.1 | 17 | 17 | 49 | 475 |
| ECOA144.1 | 20 | 122 | 149 | 459 |
| ECOA146.1 | 38 | 106 | 136 | 460 |
| ECOA147.1 | 11 | 26 | 51 | 476 |
| ECOA147.2 | 30 | 47 | 72 | 476 |
| ECOA147.3 | 31 | 68 | 92 | 476 |
| ECOA160.1 | 28 | 187 | 208 | 477 |
| ECOA160.2 | 27 | 204 | 224 | 477 |
| ECOA73.1 | 10 | 654 | 682 | 361 |
| ECOA75.1 | 18 | 944 | 970 | 364 |
| ECOA75.2 | 26 | 966 | 992 | 364 |
| ECOA75.3 | 7 | 988 | 1015 | 364 |
| ECOb2969.1 | 18 | 242 | 256 | 457 |
| ECOb2986.1 | 14 | 60 | 88 | 457 |
| SFO0179.1 | 40 | 96 | 124 | 1042 |
| SFO2968.1 | 6 | 550 | 575 | 1031 |
| SFO2968.2 | 8 | 571 | 596 | 1031 |
| SFO2968.3 | 5 | 592 | 616 | 1031 |
| SFO2972.1 | 14 | 9 | 22 | 1032 |
| SFOSepA.1 | 0 | 139 | 163 | 1007 |
| SFOSepA.2 | 1 | 159 | 183 | 1007 |
| SFOSepA.3 | 38 | 179 | 203 | 1007 |
| SFOSepA.4 | 22 | 199 | 220 | 1007 |
| SFOSepA.5 | 13 | 549 | 576 | 1007 |
| SFOSepA.6 | 3 | 572 | 599 | 1007 |
| SFOSepA.7 | 0 | 595 | 622 | 1007 |
| ECO0041.1 | 11 | 70 | 88 | 302 |
| ECO0063.1 | 24 | 134 | 147 | 303 |
| ECO0321.1 | 23 | 378 | 400 | 315 |
| ECO0345.1 | 20 | 3 | 19 | 318 |
| ECO0557.1 | 15 | 103 | 119 | 323 |
| ECO0565.1 | 23 | 266 | 292 | 324 |
| ECO0594.1 | 20 | 179 | 193 | 325 |
| ECO0602.1 | 18 | 282 | 296 | 327 |
| ECO0605.1 | 19 | 718 | 740 | 328 |
| ECO0776.1 | 36 | 1 | 29 | 330 |
| ECO0776.2 | 12 | 30 | 58 | 330 |
| ECO0776.3 | 14 | 59 | 87 | 330 |
| ECO0855.1 | 26 | 54 | 69 | 332 |
| ECO0880.1 | 16 | 33 | 56 | 334 |
| ECO1140.1 | 23 | 71 | 84 | 340 |
| ECO1392.1 | 32 | 60 | 70 | 341 |
| ECO1638.1 | 10 | 17 | 41 | 344 |
| ECO1646.1 | 6 | 110 | 127 | 346 |
| ECO2062.1 | 18 | 101 | 122 | 356 |
| ECO2099.1 | 5 | 408 | 427 | 358 |
| ECO2178.1 | 5 | 42 | 61 | 359 |
| ECO2364.1 | 15 | 40 | 59 | 362 |
| ECO2491.1 | 9 | 178 | 193 | 366 |
| ECO2540.1 | 3 | 518 | 545 | 368 |
| ECO2540.2 | 9 | 541 | 568 | 368 |
| ECO2540.3 | 2 | 564 | 591 | 368 |
| ECOA012.1 | 1 | 415 | 440 | 462 |
| ECOA012.2 | 2 | 436 | 461 | 462 |
| ECOA012.3 | 3 | 457 | 482 | 462 |
| ECOA012.4 | 6 | 478 | 503 | 462 |
| ECOA016.1 | 33 | 1 | 29 | 464 |
| ECOA016.2 | 3 | 53 | 80 | 464 |
| ECOA016.3 | 1 | 76 | 103 | 464 |
| ECOA016.4 | 8 | 99 | 126 | 464 |
| ECOA017.1 | 1 | 342 | 366 | 465 |
| ECOA017.2 | 1 | 362 | 386 | 465 |
| ECOA017.3 | 0 | 382 | 406 | 465 |
| ECOA017.4 | 15 | 402 | 428 | 465 |
| ECOA018.1 | 0 | 342 | 369 | 466 |
| ECOA018.2 | 1 | 365 | 392 | 466 |
| ECOA018.3 | 1 | 388 | 415 | 466 |
| ECOA018.4 | 2 | 411 | 438 | 466 |
| ECOA018.5 | 1 | 434 | 461 | 466 |
| ECOA018.6 | 0 | 457 | 484 | 466 |
| ECOA019.1 | 8 | 530 | 558 | 467 |
| ECOA019.2 | 16 | 554 | 582 | 467 |
| ECOA019.3 | 2 | 578 | 606 | 467 |
| ECOA019.4 | 22 | 632 | 659 | 467 |

TABLE 8
Additional immunogenic proteins identified from E. coli by bacterial surface display.

| | | | No. of | Location of | | |
|---|---|---|---|---|---|---|
| | | | selected | identified | | |
| E. coli | | | clones per | immunogenic | Seq. | Seq. |
| antigenic | Putative function | | ORF and | region | ID | ID |
| protein | (by homology) | predicted immunogenic aa* | screen | (aa) | (DNA) | (Protein) |
| b3327 | putative general | 34-42, 56-87, 95-133, 136-146, 157-213, | E: 2 | 196-215 | 1337 | 1365 |
| | protein secretion | 219-235, 246-282, 313-333, | | | | |
| | protein | 358-394 | | | | |
| EAEC166 | 62K membrane antigen | 67-74, 88-94, 112-118, 127-138, | H: 4 | 27-129 | 1338 | 1366 |
| | ipaB | 155-169, 171-180, 183-190, 196-205, | | | | |
| | | 243-249, 260-271, 308-344, | | | | |
| | | 346-373, 381-414, 416-457, 473-513, | | | | |
| | | 515-524, 528-535, 539-544, | | | | |
| | | 556-566, 572-580, 585-590 | | | | |
| EAEC168 | Nickel transport ATP- | 4-16, 21-36, 38-47, 54-64, 92-103, | L: 7 | 128-153 | 1339 | 1367 |
| | binding protein nikE | 117-126, 135-155, 157-200, 202-223, | | | | |
| | | 231-239, 246-262 | | | | |
| EAEC169 | hypothetical protein | 4-10, 16-30 | L: 2 | 11-43 | 1340 | 1369 |
| EAEC170 | pitrilysin | 7-22, 52-77, 83-93, 101-111, 125-136, | C: 2 | 246-271 | 1341 | 1370 |
| (ECs3678) | | 139-157, 212-221, 231-239, | | | | |
| | | 241-247, 264-273, 275-294, 329-336, | | | | |
| | | 349-357, 365-379, 389-405, | | | | |
| | | 419-434, 439-445, 456-462, 467-481, | | | | |
| | | 493-506, 508-516, 522-545, | | | | |
| | | 547-556, 566-583, 611-627, 655-670, | | | | |
| | | 678-693, 717-724, 734-748, | | | | |
| | | 756-766, 772-790, 797-808, 815-820, | | | | |
| | | 825-831, 833-838, 851-868, | | | | |
| | | 891-917, 919-926, 933-940, 944-953 | | | | |
| EAEC172 | hypothetical protein | 4-12, 14-31, 42-59, 61-69, 73-83, | A: 1 | 660-694 | 1342 | 1371 |
| (ECs3900) | | 96-103, 140-149, 153-165, 180-187, | | | | |
| | | 199-208, 222-230, 232-240, 248-254, | | | | |
| | | 256-270, 274-283, 289-299, | | | | |
| | | 302-317, 322-328, 332-345, 351-365, | | | | |
| | | 387-396, 419-432, 441-447, | | | | |
| | | 453-466, 487-505, 508-524, 560-571, | | | | |
| | | 580-590, 592-605, 625-633, | | | | |
| | | 639-647, 652-658, 671-679, 722-728 | | | | |
| EAEC173 | yhcM | 4-13, 26-39, 53-59, 68-74, 88-95, | C: 2 | 79-101 | 1343 | 1372 |
| (ECs4105) | | 102-119, 125-132, 136-150, 153-162, | | | | |
| | | 165-175, 188-228, 238-245, | | | | |
| | | 268-283, 285-307, 317-324, 326-343, | | | | |
| | | 350-359 | | | | |
| EAEC174 | periplasmic dipeptide | 10-37, 55-68, 71-78, 92-98, 115-122, | A: 2 | 187-224 | 1344 | 1373 |
| (ECs4424) | transport protein | 131-138, 149-158, 163-170, | | | | |
| | precursor dppA | 172-189, 212-219, 239-257, 259-271, | | | | |
| | | 289-302, 304-320, 322-340, | | | | |
| | | 359-366, 373-384, 400-412, 444-453, | | | | |
| | | 460-474, 485-527 | | | | |
| EAEC175 | dTDPglucose 4,6- | 13-21, 27-36, 41-50, 55-64, 66-72, | C: 4 | 142-171 | 1345 | 1374 |
| (ECs4721) | dehydratase | 74-90, 103-112, 127-136, 153-164, | | | | |
| | | 166-186, 193-219, 224-242, 260-273, | | | | |
| | | 278-294, 298-306, 328-334 | | | | |
| ECs0560 | RhsD core protein with | 16-29, 31-41, 49-61, 63-81, 86-92, | E: 1 | 372-393 | 1346 | 1375 |
| | extension | 107-114, 122-135, 155-177, 195-211, | | | | |
| | | 245-252, 264-270, 273-279, | | | | |
| | | 297-322, 327-334, 339-348, 371-378, | | | | |
| | | 380-389, 402-408, 414-421, | | | | |
| | | 424-430, 452-459, 481-488, 500-506, | | | | |
| | | 543-556, 567-573, 588-594, | | | | |
| | | 608-615, 628-633, 668-675, 683-689, | | | | |
| | | 700-706, 735-750, 793-802, | | | | |
| | | 816-822, 841-846, 848-855, 858-864, | | | | |
| | | 894-913, 921-929, 941-948, | | | | |
| | | 974-990, 993-1005, 1053-1068, | | | | |
| | | 1096-1110, 1117-1123, 1126-1145, | | | | |
| | | 1149-1168, 1191-1203, 1219-1225, | | | | |
| | | 1239-1248, 1253-1265, 1297-1309, | | | | |
| | | 1356-1370, 1373-1379, 1388-1395 | | | | |
| ECs3841 | ornithine decarboxylase | 5-15, 29-37, 39-46, 51-60, 65-71, | F: 1 | 315-397 | 1347 | 1376 |
| | isozyme | 73-97, 105-131, 137-152, 154-161, | | | | |
| | | 173-185, 193-210, 214-222, 224-232, | | | | |
| | | 241-255, 266-274, 277-289, | | | | |
| | | 291-303, 307-338, 345-352, 358-371, | | | | |
| | | 389-395, 402-422, 433-440, | | | | |
| | | 444-465, 471-478, 498-513, 524-536, | | | | |
| | | 542-558, 561-576, 584-602, | | | | |
| | | 604-623, 631-639, 643-658, 667-683, | | | | |
| | | 689-716 | | | | |
| Non-ORF | | | | | | |
| ARF- | hypothetical protein | 7-25, 30-37, 39-60, 69-86 | P: 5 | 75-89 | 1348 | 1377 |
| b0289 | | | | | | |
| ARF153 | hypothetical protein | — | L: 1 | 16-30 | 1349 | 1378 |
| ARF154 | hypothetical protein | — | L: 16 | 8-27 | 1350 | 1379 |
| CRF82 | hypothetical protein | 4-22, 29-48, 50-58, 62-69, 71-78 | L: 5 | 6-36 | 1351 | 1380 |
| CRF3349 | hypothetical protein | 4-23 | E: 4 | 3-24 | 1352 | 1381 |
| CRF3370 | hypothetical protein | 4-12, 30-66 | E: 12 | 11-33 | 1353 | 1382 |
| CRF3376 | hypothetical protein | 4-9, 40-64, 68-82 | E: 9 | 54-72 | 1354 | 1383 |
| CRF3792 | hypothetical protein | 4-42 | E: 103 | 11-39 | 1355 | 1384 |
| CRF4475 | hypothetical protein | 15-24, 77-88, 90-103, 116-123, 134-144 | E: 2 | 23-33 | 1356 | 1385 |
| CRF4857 | hypothetical protein | 17-25, 47-56, 68-79 | E: 2 | 64-74 | 1357 | 1386 |
| CRF5082 | hypothetical protein | 4-12, 14-21, 27-33 | E: 2 | 7-25 | 1358 | 1387 |
| CRF5163 | hypothetical protein | 4-10, 25-37 | E: 17 | 5-23 | 1359 | 1388 |
| CRF5313 | hypothetical protein | 4-9, 14-29, 38-44, 49-67, 69-97, | E: 2 | 495-511 | 1360 | 1389 |
| | | 118-128, 135-146, 151-157, 159-172, | | | | |
| | | 196-203, 227-241, 253-259, | | | | |
| | | 262-271, 284-295, 299-309, 343-353, | | | | |
| | | 356-362, 392-406, 410-416, | | | | |
| | | 429-441, 463-478, 503-509 | | | | |
| NRF7 | hypothetical protein | 4-12, 24-33, 39-51, 57-63, 78-87 | C: 2 | 32-56 | 1361 | 1390 |
| NRF8 | hypothetical protein | 8-15, 29-40, 48-54 | C: 3 | 33-56 | 1362 | 1391 |
| NRF9 | hypothetical protein | 12-61 | E: 8 | 35-61 | 1363 | 1392 |
| NRF10 | hypothetical protein | — | B: 1 | 8-27 | 1364 | 1393 |

### REFERENCES

Altboum, Z., et al. (2001). Immun. 69:3150-8.
Altschul, S., et al. (1990). Journal of Molecular Biology 215: 403-10.
Bennett, D., et al. (1995). J. Mol Recognit 8: 52-8.
Clackson, T., et al. (1991). Nature 352: 624-8.
Cohen, M. (1991). J. Pediatr. 118: S34-9.
Devereux, J., et al. (1984). Nucleic acids research 12: 387-95.
Doherty, E., et al. (2001). Annu Rev Biophys Biomol Struct 30: 457-475.
Eisenbraun, M., et al. (1993). DNA Cell Biol 12: 791-7.
Etz, H., et al. (2001). J. Bacteriol 183: 6924-35.
Ganz, T. (1999). Science 286: 420-421.
Georgiou, G. (1997). Nature Biotechnology 15:29-34.
Hashemzadeh-Bonehi, L., et al. (1998). Mol Microbiol 30: 67-678.
Heinje, von G. (1987) e.g. Sequence Analysis in Molecular Biology, Academic Press.
Hemmer, B., et al. (1999). Nat Med 5: 1375-82.
Hoe, N., et al. (2001). J. Infect Dis 183: 633-9.
Hornef, M., et al. (2002). Nat Immunol 3: 1033-40.
Johanson, K., et al. (1995). J. Biol Chem 270: 9459-71.
Jones, P., et al. (1986). Nature 321: 522-5.
Kajava, A., et al. (2000). J. Bacteriol 182: 2163-9.
Kohler, G., et al. (1975). Nature 256: 495-7.
Kolaskar, A., et al. (1990). FEBS Lett 276: 172-4.
Lewin, A., et al. (2001). Trends Mol Med 7: 221-8.
Marks, J, et al. (1992). Biotechnology (NY) 10: 779-83.
McCafferty, J., et al. (1990). Nature 348:552-4.
Okano, H., et al. (1991). J. Neurochem 56: 560-7.
Oligodeoxynucleotides as Antisense Inhibitors of Gene Expression; CRC Press, Boca Raton, Fla. (1988).
Rammensee, H., et al. (1999). Immunogenetics 50: 213-9.
Seeger, C., et al. (1984). Proc Natl Acad Sci USA 81:5849-52.
Skerra, A. (1994). Gene 151: 131-5.
Tang, D., et al. (1992). Nature 356: 152-4.
Tempest, P., et al. (1991). Biotechnology (NY) 9:266-71.
Tourdot, S., et al. (2000). Eur J. Immunol 30: 3411-21.
Turbyfill, K., et al. (2000). Infect Immun. 68: 6624-32.
Wiley, J., et al. (1987) Current Protocols in Molecular Biology

1.-37. (canceled)

38. A hyperimmune serum-reactive antigen comprising an amino acid sequence encoded by a nucleic acid molecule of claim 38 or a fragment thereof and further defined as having an amino acid sequence of SEQ ID NO: 302-336, 338-600, 763-924, 997-1068, 1203-1336, or 1365-1393.

39. The hyperimmune serum-reactive antigen fragment of claim 38, further defined as a peptide comprising an amino acid sequence of column “predicted immunogenic aa” and/or “location of identified immunogenic region” of any of Table 1 to 4 and/or Table 8, “aa (start-stop)” of Table 6, and/or a serum reactive epitope of Table 7.

40. The hyperimmune serum-reactive antigen fragment of claim 39, further defined as comprising an amino acid sequence of amino acids: 12-19, 24-29, 37-43, 47-53, 65-72, 83-95, 112-122, 136-147, 162-168, 174-181, 189-195, 201-208, 216-221, 234-243, 270-276, 278-288, 305-316, 318-342, 350-356, 368-400, 420-428, 434-443, 471-477, 481-488, 530-535, 540-547, 566-575, 591-601, 603-609, 624-629, and/or 192-333 of SEQ ID NO: 301; 9-22, 38-46, 51-61, 66-73, 108-126, 136-154, 162-169, 177-186, 198-204, 231-254, 256-272, 277-295, 297-311, 314-328, 331-338, 379-385, 393-402, and/or 69-88 of SEQ ID NO: 302; 18-36, 43-53, 80-86, 94-110, 112-118, 152-168, 170-182, 188-198, 200-220, 225-230, 237-243, 248-259, 265-289, 298-317, 325-331, 338-344, 349-360, 382-389, 400-407, 413-419, 433-453, 494-501, 503-524, 530-536, 557-565, 574-582, 586-592, 603-629, 631-637, 643-657, 673-680, 699-705, 715-720, 737-754, 764-771, 778-793, 800-844, 853-858, 874-893, 899-905, 915-929, 957-965, 134-148, 525-552, and/or 821-920 of SEQ ID NO: 303; 11-22, 28-34, 40-45, 65-86, 99-107, 115-125, 132-141, 143-150, 158-190, 203-211, 216-239, 246-257, 259-270, 272-279, 286-306, 313-332, 338-364, 369-380, 387-397, 410-418, 422-435, 449-455, 467-510, 515-521, 532-538, 547-563, 251-323, and/or 368-389 of SEQ ID NO: 304; 7-20, 28-45, 51-66, 81-104, 108-115, 124-137, 149-155, 161-206, 209-214, 222-239, 250-262, 276-282, 309-343, 351-363, 365-386, 405-413, 435-440, 446-454, 458-466, 470-477, 482-492, and/or 235-269 of SEQ ID NO: 305; 12-43, 51-60, 65-74, 76-86, 102-108, 110-118, 129-139, 146-160, 164-172, 180-192, 195-208, 212-220, 228-250, 252-267, 271-277, 281-288, 296-313, 344-353, 364-379, 381-387, 394-414, 435-443, 451-460, 468-474, 484-491, 500-510, 541-556, 560-586, 604-618, 635-641, 647-657, 668-705, 715-727, 729-734, 740-745, 760-780, 803-809, and/or 752-825 of SEQ ID NO: 306; 16-23, 66-90, 98-110, 125-131, 144-150, 194-200, 213-219, 221-232, 237-256, 263-281, 293-298, 311-318, 326-337, 339-354, 373-389, 396-402, 404-421, 427-439, 441-448, 452-462, 467-479, 508-530, 534-541, 544-550, 562-569, 575-581, 583-592, 595-628, 636-656, 658-672, 674-680, 687-697, 715-721, 731-736, 739-749, 754-761, 771-788, 790-797, 813-824, and/or 623-653 of SEQ ID NO: 307; 14-42, 51-57, 66-77, 84-96, 103-111, 129-148, 158-193, 198-208, 212-222, 242-262, and/or 31-133 of SEQ ID NO: 308; 4-23, 36-62, 65-84, 98-104, 128-135, 144-161, 175-204, 219-240, 250-264, 266-278, 280-290, and/or 119-152 of SEQ ID NO: 309; 13-26, 33-45, 50-60, 75-81, 97-105, 123-131, 138-145, 158-166, 168-177, and/or 66-170 of SEQ ID NO: 310; 20-26, 35-50, 52-67, 85-100, 106-149, 189-199, 202-208, 217-226, 236-244, 270-294, 310-332, 340-347, 350-356, 364-373, 375-380, 401-428, 438-445, 477-493, 513-548, 555-564, 568-596, 600-612, 650-665, 667-674, 680-687, 696-707, 715-722, 728-764, 779-784, 795-809, 813-820, 837-843, 858-864, 885-891, 894-900, 907-917, 922-935, 941-946, 970-977, 979-986, 1022-1032, and/or 881-924 of SEQ ID NO: 311; 13-21, 48-57, 72-83, 105-119, 125-133, 146-153, 170-177, 221-239, 245-274, 283-292, 299-305, 317-329, 335-343, 358-367, 374-380, 399-407, 430-438, 449-454, 473-479, 483-505, 517-527, 531-537, 554-560, 586-599, 601-616, 623-629, 639-647, 649-654, 658-667, 669-676, 690-709, 714-729, and/or 277-306 of SEQ ID NO: 312; 14-28, 34-40, 45-54, 69-83, 86-100, 116-123, 135-143, 146-161, 168-179, 187-200, 203-225, 237-250, 255-265, 271-292, 298-314, and/or 104-138 of SEQ ID NO: 313; 4-28, 36-42, 78-85, 106-122, 130-135, 144-150, 161-175, 180-190, 194-200, 226-234, 256-265, 274-294, 309-316, 324-333, 373-379, 382-389, 398-404, 407-416, 422-446, 451-462, 530-541, and/or 448-483 of SEQ ID NO: 314; 5-50, 53-64, 75-80, 110-116, 119-125, 131-148, 150-156, 192-217, 224-229, 260-267, 275-281, 283-300, 305-310, 323-343, 358-365, 400-406, 422-429, 447-464, 498-503, 512-518, 520-526, 530-537, 552-562, 614-623, 697-703, 705-711, 719-727, 729-743, 745-753, 788-796, 802-810, 813-834, and/or 377-400 of SEQ ID NO: 315; 4-26, 55-61, 64-70, 74-99, 107-119, 128-134, 137-154, 167-178, and/or 108-137 of SEQ ID NO: 316; 12-54, 70-76, 117-125, 135-143, 196-202, 205-213, 243-261, 263-269, 278-298, 312-318, 320-326, 334-346, 358-368, 377-389, 396-404, 414-423, 429-438, 448-455, 483-490, 540-549, 557-563, 592-599, 601-609, 622-628, 632-653, 656-692, 695-712, 714-730, 760-766, 775-786, 788-803, 807-814, 820-831, 848-854, 875-886, 892-899, 911-917, 919-925, 927-935, 954-974, 980-992, 1017-1026, 1032-1044, 1074-1079, 1082-1089, 1098-1108, 1115-1120, 1129-1137, 1139-1145, 1161-1170, 1189-1195, 1197-1212, 1219-1225, 1234-1250, 1267-1283, 1302-1318, 1321-1329, 1362-1368, 1377-1388, 1395-1408, and/or 894-924 of SEQ ID NO: 317; 58-76, 82-87, 95-103, 107-114, 122-136, 139-148, 150-162, 165-172, 184-190, 203-220, 228-238, 245-258, 260-267, 288-295, 299-328, 333-352, 357-386, 424-429, and/or 2-20 of SEQ ID NO: 318; 5-26, 59-66, 68-74, 81-87, 105-116, 122-132, 144-160, 185-212, 217-223, 228-234, 241-252, 269-274, 291-313, 318-326, 335-342, 352-358, 368-375, 397-404, 411-422, 431-439, 458-472, 474-485, 493-499, 509-519, 521-527, 530-558, 563-579, 590-601, and/or 554-596 of SEQ ID NO: 319; 6-12, 18-28, 39-45, 69-102, 120-128, 137-147, 164-170, 173-179, 223-230, 236-254, 265-277, 320-326, 350-368, 376-400, 404-416, 441-448, 462-477, and/or 178-285 of SEQ ID NO: 320; 4-19, 21-47, 52-57, 59-73, 79-86, 88-95, 100-108, 114-129, 136-143, 145-151, 176-182, 235-242, 248-258, 273-281, 301-308, 310-316, 329-340, 347-354, 363-380, 384-400, 407-415, 430-441, 469-480, 491-504, 507-526, 530-540, 547-554, 563-579, 609-617, 620-626, 630-636, 643-655, 665-680, 706-714, 718-725, 729-740, 747-754, 756-779, 790-803, 806-816, 818-824, 829-840, 842-853, 862-877, 901-912, 928-939, 941-952, 961-978, 988-999, 1026-1037, 1067-1078, 1080-1087, 1089-1098, 1104-1115, 1117-1124, 1128-1139, 1143-1151, 1155-1176, 1180-1186, 1205-1211, 1218-1224, 1228-1242, 1244-1251, 1258-1278, 1280-1287, 1290-1298, 1304-1326, 1331-1341, 1363-1378, 1392-1399, 1407-1415, 1430-1443, 1445-1454, 179-265, and/or 1343-1368 of SEQ ID NO: 321; 4-24, 31-37, 61-75, 83-89, 94-102, 117-123, 130-143, 184-191, 203-210, 212-228, 270-284, 286-292, 301-307, 312-319, 329-335, and/or 238-319 of SEQ ID NO: 322; 4-16, 22-35, 39-44, 50-59, 65-73, 86-104, 108-117, 128-137, 139-147, 153-159, 165-171, 185-192, 198-223, and/or 102-120 of SEQ ID NO: 323; 8-16, 23-30, 32-40, 45-50, 61-68, 81-89, 91-114, 121-129, 131-149, 161-185, 191-200, 217-224, 229-249, 253-262, 266-273, 282-289, 297-303, and/or 265-282 of SEQ ID NO: 324; 10-48, 64-71, 81-88, 100-112, 130-140, 153-170, 177-184, 197-202, 236-250, 266-272, 284-292, 294-300, 306-318, 320-326, 346-357, 379-387, 389-396, 405-416, 424-435, 447-453, 474-483, 501-510, 529-536, 550-568, 582-594, 606-611, 625-631, 633-645, 664-672, 685-692, 703-711, 730-745, 761-775, 782-790, 792-804, 816-825, 827-834, 840-866, and/or 178-193 of SEQ ID NO: 325; 11-25, 39-57, 69-94, 100-107, 118-155, 158-171, 189-201, 226-233, 236-245, 249-263, 268-277, 287-312, 315-329, 333-342, 351-357, 364-374, 382-388, 399-407, 419-449, 454-471, 486-492, 494-504, 515-541, 547-552, 578-600, 611-623, 625-641, 651-657, 678-692, 699-709, 713-720, 746-752, 772-781, 791-801, 829-844, 880-893, 900-910, 915-923, 936-942, 953-970, 88-167, and/or 804-839 of SEQ ID NO: 326; 6-16, 19-40, 55-61, 67-92, 103-108, 125-142, 154-162, 190-196, 236-247, 257-272, 280-290, 310-320, 331-343, 367-375, and/or 281-297 of SEQ ID NO: 327; 42-48, 63-116, 151-172, 181-187, 189-196, 200-205, 217-232, 256-261, 288-319, 326-352, 360-374, 380-404, 412-435, 453-466, 474-485, 500-507, 514-519, 525-530, 558-564, 568-582, 590-595, 600-610, 633-640, 666-671, 694-702, 715-722, 730-737, 757-768, 774-785, 820-827, 832-848, 873-878, 883-890, 906-915, 918-930, 937-944, 948-956, 960-966, 970-978, 1023-1040, 1049-1056, 1065-1071, 1085-1090, 1105-1117, 1122-1132, 1165-1171, 1186-1195, 1210-1216, 1309-1342, 1345-1352, 1354-1360, 1375-1385, 1400-1407, 1414-1421, 1430-1439, 1446-1467, 1479-1485, 1522-1530, 1565-1572, 1577-1586, 1596-1608, and/or 717-741 of SEQ ID NO: 328; 5-11, 17-24, 26-32, 36-43, 50-61, 67-73, 91-102, 111-126, 133-148, 154-161, 167-173, 179-195, 208-223, 230-252, 270-286, 292-306, 308-347, 352-371, 373-380, 386-395, 404-410, 418-431, 436-444, 447-460, 463-477, 486-492, 522-533, 545-553, and/or 36-133 of SEQ ID NO: 329; 4-23, 68-78, 100-107, 135-149, 152-159, and/or 1-88 of SEQ ID NO: 330; 5-12, 18-27, 35-55, 68-95, 100-109, 117-122, 129-135, 157-162, and/or 37-98 of SEQ ID NO: 331; 5-52, 64-80, 86-106, 108-155, 175-190, 223-231, 234-248, and/or 53-70 of SEQ ID NO: 332; 25-46, 59-64, 69-75, 83-90, 93-100, 107-115, 124-135, 151-177, 183-189, 194-206, 209-215, 219-224, 251-263, 267-276, 305-311, 318-327, 332-338, 350-356, 380-396, 406-412, 414-423, 431-437, 453-461, 463-481, 483-491, 505-510, 513-523, 528-545, 568-575, and/or 226-275 of SEQ ID NO: 333; 5-29, 37-43, 47-54, 61-70, and/or 31-65 of SEQ ID NO: 334; 10-35, 42-59, 65-70, 76-85, 92-104, 149-155, 184-191, 234-243, 248-259, 268-277, 383-389, 391-398, 410-430, 445-454, 488-504, 518-523, 530-538, 574-590, 615-623, 627-633, 652-660, 662-670, 674-683, 703-714, 720-728, 731-737, 751-757, and/or 547-572 of SEQ ID NO: 335; 5-12, 39-51, 57-64, 67-84, 86-108, 124-130, 138-159, 167-179, 181-202, 226-235, and/or 133-214 of SEQ ID NO: 336; 12-20, 29-40, 57-77, 79-88, 97-103, 111-117, 119-137, 174-200, 202-218, 221-229, 231-238, 240-246, 254-264, 266-280, 296-308, 321-331, and/or 23-54 of SEQ ID NO: 337; 7-17, 19-54, 66-101, 114-133, 146-182, 193-210, 219-226, 232-238, 244-250, 253-261, 266-279, and/or 1-31 of SEQ ID NO: 338; 4-11, 17-32, 38-58, 68-111, 113-130, 132-186, 200-212, 219-227, 240-249, 256-265, 270-278, 285-305, 311-317, 328-341, 343-351, 373-386, 389-414, 419-433, 439-504, 510-535, 553-564, 588-594, 599-604, 609-618, 620-631, 635-657, 664-670, 684-703, 705-717, and/or 661-695 of SEQ ID NO: 339; 5-33, 67-78, 122-129, 141-150, 172-185, 201-209, 217-223, 235-252, 289-295, 303-316, 355-368, 383-389, 398-406, 426-437, 445-451, 459-467, 479-496, 512-517, 523-530, 535-562, 577-584, 590-605, 610-616, 618-632, 644-654, 663-669, 680-688, 70-85, and/or 271-403 of SEQ ID NO: 340; 4-53, 55-62, and/or 59-71 of SEQ ID NO: 341; 26-38, 43-63, 67-76, 78-98, 105-112, 115-121, 132-144, 148-153, 179-184, 194-203, 239-245, 261-278, 282-315, and/or 231-283 of SEQ ID NO: 342; 13-22, 24-30, 49-61, 65-72, 90-97, 99-105, 115-131, 152-160, 165-171, 176-188, 202-221, 231-250, 255-274, 280-286, 288-296, 331-337, 339-347, 350-358, 374-385, 391-408, 418-427, 438-453, 468-476, 482-490, 497-506, 526-532, 534-583, 696-702, 713-719, 730-748, 750-758, 762-776, 802-808, 825-857, 864-950, 963-1004, 1015-1023, 1046-1058, and/or 571-665 of SEQ ID NO: 343; 5-13, 18-24, 29-45, 51-58, 78-85, 87-94, 109-117, 122-128, 146-161, 175-189, and/or 1-83 of SEQ ID NO: 344; 5-17, 40-46, 50-65, 73-86, 89-98, 114-139, 151-157, 165-173, 186-195, 197-213, 215-227, 245-260, 310-315, 364-369, 405-415, 428-436, 456-464, 471-482, 507-514, 518-531, 539-565, 648-654, 681-687, 690-707, 720-731, 743-756, 764-771, 801-806, 815-828, 830-836, 204-283, and/or 287-363 of SEQ ID NO: 345; 6-27, 33-40, 62-78, 83-89, 91-99, 102-109, 115-134, 150-156, 160-170, 172-204, 233-244, and/or 109-127 of SEQ ID NO: 346; 4-11, 17-25, 31-38, 55-80, 105-113, 115-123, 160-171, 195-201, 210-222, 254-264, 290-296, 301-310, 315-321, 329-334, 349-358, 377-394, 403-409, 424-432, 452-460, 466-474, 480-495, 510-516, 527-539, 554-562, 568-579, 587-592, 599-608, 627-635, 637-646, 661-668, 702-709, 723-736, 744-766, 768-778, 785-802, 815-821, 828-835, 862-868, 876-888, 892-906, 908-918, 921-950, 192-275, and/or 394-430 of SEQ ID NO: 347; 18-26, 33-78, 80-87, 120-128, 141-182, 184-208, 251-257, 269-300, 305-311, and/or 208-230 of SEQ ID NO: 348; 11-72, 84-91, 99-109, 112-120, 143-155, 162-183, 188-197, 222-231, and/or 30-176 of SEQ ID NO: 349; 4-17, 41-56, 61-67, 74-109, 142-149, 158-185, 193-210, 216-236, 241-249, and/or 84-162 of SEQ ID NO: 350; 8-45, 135-140, 176-182, 189-196, 206-216, 218-235, 260-269, 272-278, 307-313, 331-344, 352-359, 371-395, 403-414, 416-422, 426-438, 451-470, 478-484, 493-502, 504-511, 514-525, 527-534, and/or 104-180 of SEQ ID NO: 351; 6-25, 49-59, 65-94, 107-115, 117-124, 135-151, 176-185, 203-209, and/or 145-173 of SEQ ID NO: 352; 5-15, 46-56, 58-81, 83-111, 118-138, 146-158, 165-175, and/or 9-46 of SEQ ID NO: 353; 7-15, 36-43, 54-60, 65-73, 88-94, 107-113, 122-128, 134-141, 162-171, 182-216, 218-235, 249-258, 266-278, 290-301, 308-338, 362-368, and/or 100-141 of SEQ ID NO: 354; 4-14, 19-24, 27-36, 38-51, 59-73, 90-96, 102-121, 138-150, 157-174, 176-202, 212-225, 229-241, 250-258, 261-268, 279-291, 293-310, 319-338, 358-368, 371-389, 393-398, 404-413, 416-433, 435-442, 458-471, and/or 327-355 of SEQ ID NO: 355; 10-15, 32-49, 61-69, 97-103, 128-134, 143-154, 164-179, and/or 99-124 of SEQ ID NO: 356; 6-37, 40-48, 56-68, 108-127, 135-141, 145-163, 170-179, 207-216, 224-234, 238-244, 249-261, 266-283, 352-363, 371-378, 380-392, 447-454, 468-489, 497-503, 505-510, and/or 434-465 of SEQ ID NO: 357; 6-33, 46-68, 78-84, 120-131, 135-142, 183-188, 201-219, 227-233, 236-244, 246-252, 282-295, 307-315, 335-350, 392-399, 409-414, 427-433, 435-447, 468-482, 487-494, 500-506, 529-537, 543-550, 555-575, 577-584, 606-611, 619-625, 637-651, 682-711, 713-727, 729-738, 756-764, 783-795, 801-815, 817-830, 833-847, 861-890, 898-904, 909-928, and/or 406-428 of SEQ ID NO: 358; 7-17, 36-53, and/or 41-65 of SEQ ID NO: 359; 4-10, 17-24, 28-34, 42-49, 55-61, 70-106, 112-127, 135-142, 154-172, 178-184, 187-201, 213-219, 225-230, 235-246, 253-260, 267-302, 318-331, 339-353, 363-373, 376-387, and/or 258-268 of SEQ ID NO: 360; 8-17, 29-43, 45-52, 58-69, 87-100, 102-111, 148-163, 172-187, 190-208, 210-227, 232-239, 245-253, 258-263, 286-299, 313-334, 346-362, 373-388, 391-411, 425-430, 434-446, 457-489, 496-502, 518-524, 537-546, 555-560, 602-610, 637-646, 676-689, 698-704, 706-742, 750-778, 780-791, 806-842, 864-879, 881-888, 890-899, 901-908, 910-921, 941-947, 953-959, 967-980, 990-995, 1000-1061, 1073-1079, 1081-1092, 1096-1118, 1121-1185, 1195-1209, 1219-1232, 1237-1243, 1250-1274, 1276-1282, 1302-1317, 1324-1333, 1339-1344, 1349-1361, 1370-1376, 1406-1413, 1415-1427, 1433-1450, 1453-1469, 1473-1478, 1482-1495, 1509-1517, 1519-1526, and/or 611-689of SEQ ID NO: 361; 7-29, 74-83, 101-107, 115-124, 127-142, 166-184, 209-215, 222-232, 245-252, 255-262, and/or 40-60 of SEQ ID NO: 362; 4-14, 16-32, 42-47, 65-71, 82-109, 128-145, 158-171, 177-191, 197-228, 230-236, and/or 138-167 of SEQ ID NO: 363; 4-22, 47-59, 61-71, 76-82, 96-103, 119-146, 165-172, 174-188, 191-202, 214-232, 240-247, 267-273, 278-293, 303-323, 326-340, 348-353, 365-387, 389-398, 405-411, 416-421, 423-451, 453-480, 482-495, 507-512, 518-529, 537-545, 563-570, 581-587, 591-597, 611-624, 626-634, 637-647, 671-692, 694-725, 735-742, 747-768, 786-825, 859-875, 887-894, 897-909, 915-938, 943-954, 967-976, 984-1001, 1006-1015, and/or 943-1016 of SEQ ID NO: 364; 4-14, 24-34, 47-69, 81-90, 98-112, 144-153, 161-169, 189-196, 202-208, 213-220, 243-249, 256-262, 265-271, 279-285, 299-307, 310-324, 326-345, 356-369, 397-416, 424-429, 432-441, and/or 361-403 of SEQ ID NO: 365; 4-22, 27-40, 44-54, 77-91, 112-127, 155-161, 196-207, 210-216, 228-234, and/or 171-210of SEQ ID NO: 366; 4-9, 14-23, 50-56, 59-68, 77-102, 111-120, 126-152, 161-167, 174-180, 189-202, 204-228, 237-245, 259-266, 278-285, 300-309, and/or 113-157 of SEQ ID NO: 367; 6-26, 31-37, 41-47, 62-69, 71-93, 106-119, 126-136, 167-180, 196-202, 210-216, 242-254, 258-264, 272-286, 292-298, 300-309, 312-322, 346-352, 354-380, 385-392, 401-420, 434-449, 451-459, 465-473, 497-514, 555-562, 566-574, 604-612, 642-648, 657-669, and/or 517-643 of SEQ ID NO: 368; 23-35, 71-77, 94-100, 134-140, 157-163, 185-191, 228-237, 255-265, 269-283, 310-315, 334-340, 366-392, 395-400, 404-411, 423-428, 434-445, 451-458, 468-478, 496-508, 512-519, 558-563, 565-582, 11-149, and/or 507-577 of SEQ ID NO: 369; 14-20, 35-48, 53-63, 71-77, 95-101, 114-121, 123-130, 144-151, 153-160, 162-170, 187-197, 201-211, and/or 23-52 of SEQ ID NO: 370; 7-17, 24-44, 63-70, 88-99, and/or 1-78 of SEQ ID NO: 371; 21-39, 46-53, 68-96, 107-113, 118-124, 126-135, 158-185, 196-202, 204-213, 219-226, 246-253, 267-275, 277-285, 299-317, 319-338, 404-410, 421-428, 435-463, 92-170, and/or 178-199 of SEQ ID NO: 372; 28-43, 47-55, 59-68, 72-79, 106-112, 121-139, 151-160, 168-175, 177-183, 194-212, 223-229, 232-248, 254-263, 270-276, 317-323, 331-338, 342-356, 363-369, 378-391, 415-424, 432-441, 443-456, 464-470, 499-505, 521-527, 534-552, 586-599, 624-634, 639-647, 651-667, 685-690, 694-702, 711-731, 733-744, 752-776, 784-791, 801-807, 837-859, 879-890, 906-914, 918-924, 926-940, 945-958, 965-971, 980-1002, 1010-1016, 1018-1028, 1034-1044, 1046-1053, 1065-1075, 1079-1092, 1095-1104, 1125-1142, 1154-1162, 1176-1181, 1194-1207, 1233-1244, 1252-1261, 1267-1274, 1283-1288, 1318-1324, 1327-1342, and/or 403-455 of SEQ ID NO: 373; 17-25, 32-77, 82-91, 100-128, 163-169, 189-207, 211-218, 227-232, 239-245, 255-260, 278-300, 311-325, 342-356, 382-390, 393-401, 416-460, 467-487, 491-497, 505-512, 516-532, 551-565, 568-575, 594-601, 610-632, 638-643, 647-670, 672-685, 699-710, 712-726, and/or 290-399 of SEQ ID NO: 374; 4-39, 56-73, 107-128, 134-142, 144-153, 155-183, 198-203, 205-212, 215-223, 232-244, 248-265, 273-292, 294-301, 304-311, 322-329, 338-343, 369-378, 397-404, 408-416, 420-426, 436-443, and/or 177-199 of SEQ ID NO: 375; 4-22, 25-31, 35-41, 53-61, 74-83, 101-145, 157-162, 199-216, 247-257, 266-276, 282-289, 291-298, 306-313, 324-335, 345-353, 360-368, 392-400, 404-421, 432-445, 455-462, 478-486, 494-499, 501-511, 525-551, 554-561, 581-588, 600-613, 637-661, 669-676, 684-697, 699-705, 720-730, 746-751, and/or 393-543 of SEQ ID NO: 376; 11-25, 65-79, 89-97, 106-112, 115-121, 126-132, 134-141, 218-230, 255-260, 287-294, 304-309, 328-334, 339-345, 347-363, 366-382, 421-428, 457-463, 471-477, 484-492, 504-511, 513-518, 547-554, 559-572, 598-604, 617-628, 641-647, 658-665, 691-696, 701-714, 744-751, 760-770, 774-780, 792-801, 805-817, and/or 635-686 of SEQ ID NO: 377; 5-25, 31-39, 72-79, 93-102, 104-110, 122-132, 138-146, 157-189, 192-198, 205-214, 226-233, 240-248, 269-275, 282-298, 304-310, 313-327, 342-348, and/or 74-108 of SEQ ID NO: 378; 7-34, 44-50, 54-64, 90-99, 101-106, 111-123, 156-175, 182-212, 224-232, 235-247, 249-264, 266-273, 306-321, 326-333, 343-351, 359-365, 370-403, 424-455, 466-477, 481-495, 503-511, 516-531, 534-543, 555-573, 578-600, 638-650, 657-671, 677-682, 686-692, 698-710, 721-729, 732-741, 752-763, 773-784, 786-809, 816-821, 829-849, 885-894, 911-920, 931-940, 942-949, 954-962, 979-986, 988-995, 1007-1016, 1034-1039, 1060-1065, 1076-1093, 1131-1137, 1144-1152, 1160-1165, 1170-1181, 1186-1196, 1220-1260, 1271-1280, 1287-1295, 1318-1328, 1346-1356, 1361-1367, 1378-1392, 1401-1407, 1412-1420, 1426-1443, 1478-1489, 1491-1499, 1501-1525, and/or 1352-1387 of SEQ ID NO: 379; 18-53, 64-93, 95-105, 124-135, 143-148, 155-161, 163-171, 184-198, 238-245, 258-271, 273-284, 287-292, 302-310, 312-320, 322-341, 349-365, 377-403, 407-414, 417-423, 444-453, 455-469, 471-495, 503-511, 536-557, 579-586, 588-609, 619-626, 632-638, 643-649, 656-663, 669-680, 682-688, 699-714, 729-739, 755-761, 768-776, 781-793, 801-815, 821-826, 833-842, 863-869, and/or 7-84 of SEQ ID NO: 380; 8-15, 24-40, 51-65, 78-89, 102-111, 117-154, 164-177, 181-192, 198-209, 216-222, 230-237, 241-248, 254-268, 285-293, 298-321, 331-338, 366-373, 379-389, 392-415, 429-439, 441-451, 453-459, 471-486, 489-501, 524-535, and/or 1-26 of SEQ ID NO: 381; 10-18, 26-38, 48-54, 60-69, 77-83, 88-95, 119-126, 133-169, 172-185, 193-206, 214-225, 236-250, 255-261, 269-275, 278-301, 320-329, 336-341, 345-353, 356-369, 389-397, and/or 64-88 of SEQ ID NO: 382; 27-32, 37-50, 68-82, 84-108, 134-145, 147-154, 162-170, 172-182, 194-200, 205-224, 232-270, 293-299, 312-328, and/or 86-134 of SEQ ID NO: 383; 7-13, 18-44, 64-74, 81-86, 94-104, 134-148, 153-159, 174-183, 204-225, 228-243, 248-255, 283-295, 297-303, 320-347, and/or 100-141 of SEQ ID NO: 384; 4-27, 36-42, 55-62, 64-73, 92-106, 112-118, 120-127, 135-154, 170-179, 242-257, 270-277, 286-325, 335-341, 359-365, 381-387, 410-440, 470-478, 520-528, 543-553, 575-582, 603-612, 617-623, 628-649, 657-663, 685-691, 693-699, 703-708, 712-719, 740-747, 755-763, 766-782, 800-809, 811-833, 835-851, 856-862, 864-876, and/or 344-369 of SEQ ID NO: 385; 4-10, 15-24, 26-53, 55-71, 78-83, 90-113, 128-148, 156-163, 165-179, 203-213, 228-239, 250-259, 277-285, 292-314, 322-330, 334-340, 345-360, 381-396, 404-409, 416-427, and/or 204-232 of SEQ ID NO: 386; 4-17, 21-30, 42-49, 56-63, 67-73, 78-87, 92-99, 105-111, 122-130, 151-160, 168-197, 209-226, 243-276, 286-293, 295-301, 306-319, 322-332, 335-342, and/or 104-126 of SEQ ID NO: 387; 4-10, 12-23, 28-34, 37-60, 65-84, 97-103, 113-127, 135-143, 182-187, 200-223, 227-233, 236-271, 274-279, 282-287, 293-299, 314-329, 334-358, and/or 300-362 of SEQ ID NO: 388; 20-32, 37-46, 48-65, 75-83, 86-95, 121-133, 138-151, 183-190, 199-205, 216-227, and/or 1-38 of SEQ ID NO: 389; 9-25, 29-48, 50-100, 102-126, 131-149, 167-173, 210-217, 224-256, 259-270, 275-292, 295-301, 308-313, 319-335, 337-359, 362-382, 393-423, 436-449, 468-476, 481-487, 492-500, 526-534, 537-548, 560-567, 569-579, 590-598, 604-613, 629-636, 644-656, and/or 506-577 of SEQ ID NO: 390; 25-45, 53-78, 80-102, 116-128, 161-167, 180-186, 193-219, 235-258, 261-268, 291-318, and/or 214-233 of SEQ ID NO: 391; 4-18, 25-31, 33-39, 47-53, 64-92, 97-106, 123-129, 134-146, 165-171, 173-190, 192-213, 226-239, 251-273, 283-298, 316-324, 339-345, 350-356, 361-376, 400-408, 418-440, 444-451, 476-481, 505-516, 524-542, 555-563, 581-594, 607-629, 634-641, 647-670, 711-719, 728-738, 755-765, 772-780, 800-815, 822-833, 842-852, 860-865, 874-880, 891-913, 926-938, 941-946, 961-978, 984-990, 1013-1024, 1052-1092, 1099-1111, 1120-1140, 1153-1168, 1170-1190, 1193-1211, 1221-1233, 1253-1264, 1268-1274, 1283-1289, 1295-1300, 1303-1327, 1338-1351, 1362-1368, 1391-1396, 1403-1416, 1429-1436, 1471-1477, 1483-1513, 1526-1555, 1585-1591, 1596-1630, 1632-1639, and/or 299-326 of SEQ ID NO: 392; 10-25, 34-54, 57-67, 77-96, 111-121, 127-139, 151-157, 161-179, 183-198, 201-219, 233-239, 247-252, 268-276, 283-294, 299-309, 319-324, and/or 156-268 of SEQ ID NO: 393; 6-28, 34-45, 64-79, 88-95, 98-115, 120-141, 159-167, 174-179, 186-192, 198-208, 216-225, 232-246, 248-273, 275-283, 291-299, 304-316, 370-381, 386-393, 401-417, 421-445, 460-468, 470-487, 497-509, 511-535, 542-558, 564-574, 603-609, 619-648, 661-675, 682-694, 720-738, 742-759, 762-788, 793-805, 825-851, 885-893, 898-906, 918-935, 941-953, 971-978, 986-993, 1001-1017, 1019-1026, 1050-1070, 1072-1089, 1097-1102, 1107-1121, and/or 934-1003 of SEQ ID NO: 394; 6-13, 31-38, 47-60, 71-102, 107-124, 128-155, 173-180, 213-220, and/or 202-243 of SEQ ID NO: 395; 4-38, 49-71, 75-85, 110-115, 168-173, 202-210, 221-228, 240-245, 258-264, 302-316, 348-362, 386-391, 456-462, 474-483, 494-499, 511-516, 523-528, 533-539, 549-557, 579-585, 587-593, 618-625, 627-634, 654-660, 664-670, 682-688, 697-702, 729-735, 783-793, 804-812, 817-829, 862-868, 908-920, 954-960, 1000-1006, 1008-1031, 1044-1050, 1069-1077, 1079-1084, 1097-1118, 1139-1146, 1152-1158, 1165-1176, 1181-1186, 1201-1213, 1261-1267, 1272-1280, 1282-1289, 1358-1364, 1373-1382, 1390-1400, 1443-1450, 1497-1505, 1530-1552, 1560-1568, 454-483, and/or 1142-1349 of SEQ ID NO: 396; 4-13, 20-34, 47-53, 58-65, 76-82, 89-106, 139-160, 165-182, 191-205, and/or 10-41 of SEQ ID NO: 397; 31-42, 59-75, 91-102, 104-123, 147-153, 172-184, 193-206, 257-266, 306-316, 318-329, and/or 4-34 of SEQ ID NO: 398; 5-14, 26-35, 38-45, 54-60, 63-79, 121-127, 137-145, 152-162, 167-173, 175-183, 191-202, 218-228, 238-263, 278-295, 303-316, 320-335, 337-345, 359-365, 382-400, and/or 64-148 of SEQ ID NO: 399; 4-17, 31-39, 46-61, 68-73, 76-97, 128-139, 150-156, 166-172, 174-182, 184-215, 219-225, 238-245, 249-262, and/or 187-223 of SEQ ID NO: 400; 4-23, 30-41, 44-53, 58-70, 82-91, 107-114, 122-129, 148-155, 201-207, 223-232, and/or 1-69 of SEQ ID NO: 401; 4-16, 28-41, 44-52, 60-66, 73-82, 92-101, 108-114, 133-138, 145-155, 177-185, 194-202, and/or 89-130 of SEQ ID NO: 402; 4-9, 21-39, 72-78, 82-88, 99-131, 136-143, 151-162, 164-187, 189-204, 208-216, 223-229, 232-240, 246-256, 269-283, 288-299, 311-321, 328-335, and/or 209-237 of SEQ ID NO: 403; 4-14, 36-48, 66-73, 75-89, 95-103, 115-123, 128-133, 140-145, 151-158, 165-176, 178-188, 224-254, 267-278, 289-297, 302-311, and/or 178-262 of SEQ ID NO: 404; 19-25, 55-70, 76-82, 88-107, 114-129, 136-145, 154-177, 205-219, 227-233, and/or 1-35 of SEQ ID NO: 405; 26-33, 39-45, 50-62, 76-85, 87-101, 116-131, 142-152, 154-186, 193-199, 201-217, 221-243, 266-272, 281-298, 324-330, 335-342, 345-355, 375-383, 407-413, 254-315, and/or 323-407 of SEQ ID NO: 406; 4-22, 27-36, 60-69, 90-98, 107-113, 117-123, 127-134, 137-151, 154-161, 169-178, 185-192, 202-208, 214-223, 230-239, 245-255, 266-275, 307-317, 323-337, 339-353, 361-379, 385-391, 393-401, 415-422, 424-429, 434-442, 444-449, 470-480, and/or 358-400 of SEQ ID NO: 407; 4-25, 31-42, 83-101, 109-123, 127-136, 139-145, 154-166, 169-182, 194-201, 210-220, 226-237, 251-275, 277-304, 309-329, 341-362, 367-372, 377-393, 400-406, and/or 223-239 of SEQ ID NO: 408; 4-22, 29-34, 37-44, 48-78, 98-110, 127-142, 144-156, 158-165, and/or 59-127 of SEQ ID NO: 409; 4-12, 14-20, 27-34, 39-47, 51-67, 69-81, 89-97, 105-119, 121-133, 140-149, 151-161, and/or 67-105 of SEQ ID NO: 410; 6-19, 25-35, 43-48, 56-69, 73-93, 137-146, 152-161, 164-207, 212-229, 236-241, 244-250, 273-288, 292-299, 314-324, and/or 259-291 of SEQ ID NO: 411; 17-24, 34-40, 78-85, 227-233, 294-315, 327-335, 345-351, 354-359, 363-368, 388-403, 405-411, 413-419, 425-434, 462-472, 480-500, 528-536, 542-560, 566-573, 579-589, 593-606, 614-646, 651-658, 663-669, 686-726, 734-747, 754-778, 787-806, 809-825, 827-839, 876-887, and/or 80-214 of SEQ ID NO: 412; 4-9, 15-29, 38-43, 50-81, 83-96, 98-108, 116-122, 136-143, and/or 133-148 of SEQ ID NO: 413; 5-79, 98-105, 133-146, 158-182, 189-207, 213-225, 231-252, 272-278, 283-303, 312-317, 333-346, 361-367, 370-379, 387-419, 421-433, 439-453, 460-468, and/or 132-153 of SEQ ID NO: 414; 9-29, 35-40, 49-63, 69-76, 110-134, 141-147, 160-169, and/or 63-101 of SEQ ID NO: 415; 4-9, 13-20, 25-43, 50-56, 75-86, 102-115, 120-126, 128-135, 139-145, 161-166, 170-189, 202-210, 212-219, 221-232, 240-248, 252-264, 54-161, and/or 256-285 of SEQ ID NO: 416; 7-17, 19-33, 44-51, 56-70, 74-79, 85-93, 96-102, 110-117, 124-132, 140-148, 157-176, 204-248, 256-269, 289-306, 318-324, 331-339, 377-382, 389-397, 399-411, 413-420, 424-432, 436-441, 462-469, 499-506, 522-547, 549-563, 569-575, 578-594, 609-614, 621-630, 637-646, 652-685, 688-711, 713-724, 739-744, 752-783, 793-799, 811-823, 825-841, 846-855, 861-868, 874-886, 895-907, 935-966, 977-1024, 1026-1035, 1037-1055, 1063-1091, 1094-1103, 1105-1119, 1128-1149, 1160-1173, 1182-1194, 1210-1222, 1227-1234, 1244-1258, 1275-1285, 1293-1300, 1316-1322, 1336-1354, 1357-1364, 1367-1372, 1386-1392, 1403-1411, 1424-1430, 1439-1456, 1458-1469, 1485-1504, 1018-1052, and/or 1134-1262 of SEQ ID NO: 417; 4-30, 32-42, 59-65, 78-88, 104-127, 132-147, 158-171, 181-187, 195-214, 220-226, 238-269, and/or 6-90 of SEQ ID NO: 418; 10-16, 25-53, 64-74, and/or 1-83 of SEQ ID NO: 419; 4-29, 48-57, 75-86, 99-113, 146-155, 164-174, 190-201, 215-234, 236-251, and/or 101-185 of SEQ ID NO: 420; 4-9, 32-56, 58-67, 71-81, 90-95, 97-105, 112-118, 124-132, 138-144, 147-167, 170-177, 211-217, 231-241, 250-258, 260-272, 274-282, 289-296, 299-309, 319-331, 344-350, 356-362, 368-377, 381-394, 399-406, 412-430, 432-450, 459-473, 486-503, 508-515, 520-548, 564-570, 581-587, 616-623, 628-635, 638-660, 678-684, 691-696, 703-709, 716-723, 760-772, 787-795, 835-844, and/or 177-207 of SEQ ID NO: 421; 5-43, 46-81, 88-95, 137-142, 163-191, 195-203, 210-235, 241-254, 256-276, 280-288, 292-305, 307-313, 317-333, 335-343, 347-353, 357-363, 372-381, 384-389, 399-409, and/or 58-179 of SEQ ID NO: 422; 26-32, 38-44, 68-75, 85-100, 104-114, 126-132, 140-150, 153-164, 175-193, 200-209, 218-224, 226-232, 243-249, 251-260, 275-293, 304-329, 335-353, 364-479, 485-490, 500-512, 514-523, 532-556, 577-589, 622-628, 631-653, 656-678, 542-627, and/or 691-724 of SEQ ID NO: 423; 8-22, 25-30, 46-62, 67-73, 98-103, 105-114, 120-141, 144-153, 168-175, 181-193, 198-204, 208-227, 235-242, 249-258, 281-288, 291-306, 327-336, 340-361, 368-380, 389-409, 417-426, 428-435, 442-453, 468-486, 488-496, 498-509, 511-523, 540-553, 566-579, 587-603, 629-636, 677-682, and/or 170-207 of SEQ ID NO: 424; 9-25, 41-61, 68-75, 81-102, 106-141, 158-165, 173-191, and/or 32-53 of SEQ ID NO: 425; 7-26, 28-37, 43-58, 67-79, 92-99, 103-111, 118-128, 130-139, 152-165, 170-186, 192-214, 216-223, 225-251, and/or 95-122 of SEQ ID NO: 426; 4-16, 21-36, 38-47, 54-64, 92-103, 117-126, 134-155, 157-200, 202-223, 246-262, and/or 70-163 of SEQ ID NO: 427; 30-38, 45-51, 75-90, 93-114, 119-127, 134-143, 151-159, 166-180, 189-207, 217-222, 230-239, 267-276, 283-292, 324-350, 374-386, 392-403, 409-416, 418-424, 451-458, 466-477, 483-494, 501-509, 521-528, 540-549, 556-561, 573-579, 581-588, 621-626, 628-636, 654-661, 695-701, 711-717, 734-743, 751-757, 764-771, 789-798, 832-837, 860-867, 869-883, 911-917, 942-950, 958-964, 966-981, 992-999, 44-131, and/or 484-615 of SEQ ID NO: 428; 5-14, 27-42, 48-67, 71-83, 85-91, 105-112, 114-135, 139-147, 159-165, 169-185, 188-195, 199-208, 212-221, 231-253, 264-272, 275-282, 290-303, 309-319, 324-331, 340-358, 380-405, 419-425, 438-444, 450-463, 468-477, 497-514, 520-533, 549-556, 568-574, 617-626, 637-643, 661-668, 674-684, 705-713, 718-733, 735-775, and/or 541-569 of SEQ ID NO: 429; 13-19, 21-32, 45-69, 79-87, 90-109, 140-148, 156-208, 215-232, 243-274, 276-284, 288-298, 301-316, 339-347, 369-384, 405-412, 430-437, 445-457, 464-470, 475-483, 490-509, 517-524, 532-591, 609-628, 647-677, 681-709, 731-740, 752-767, 770-781, 787-793, 798-807, 825-836, 839-869, and/or 104-137 of SEQ ID NO: 430; 4-10, 33-44, 73-78, 93-101, 123-129, 135-165, 201-214, 251-261, 268-274, 285-292, 316-322, 328-336, 342-350, 353-360, 374-387, 391-399, 401-406, 417-425, 437-443, 447-454, 511-522, 530-535, 727-737, 762-774, 781-787, 803-809, 827-838, 852-859, 877-883, 901-907, 910-918, 951-956, 996-1002, 1071-1079, 1086-1096, 1098-1104, 1106-1119, 1129-1135, 1142-1148, 1155-1161, 1167-1185, 1199-1205, 1224-1232, 1242-1251, 1279-1287, 1293-1299, 1305-1321, 1372-1396, 1426-1435, 1467-1473, 1479-1492, 1526-1533, 1548-1558, 1578-1585, and/or 1520-1553 of SEQ ID NO: 431; 4-10, 36-45, 56-92, 108-114, 125-133, 137-146, 156-162, 164-186, 194-203, 225-234, 242-251, 272-283, 285-306, 310-315, 322-330, 358-371, 373-379, 389-396, and/or 95-189 of SEQ ID NO: 432; 4-32, 38-46, 66-83, 88-95, 110-118, 123-141, 169-180, 200-208, 217-225, 237-245, 247-261, 263-272, 275-282, 291-302, 310-338, 345-353, 360-369, 371-378, 386-394, 398-413, 416-422, 437-448, and/or 51-81 of SEQ ID NO: 433; 4-10, 12-38, 59-64, 81-97, 122-132, 136-142, 149-165, 180-187, 192-199, 205-216, 222-228, 244-251, 255-274, 280-286, 294-320, 327-333, 339-346, 353-359, 372-385, 402-408, 413-420, 433-440, 443-453, 456-461, 464-472, 483-495, and/or 14-27 of SEQ ID NO: 434; 4-40, 42-56, 59-73, 76-110, 115-128, 132-139, 148-170, 174-195, 197-207, 214-220, 222-236, 238-246, 252-283, 291-326, 334-413, and/or 239-261 of SEQ ID NO: 435; 4-11, 18-26, 31-47, 59-68, 74-92, 98-144, 149-158, 173-180, 200-210, 216-223, 239-250, and/or 18-66 of SEQ ID NO: 436; 4-30, 42-52, 59-67, 70-76, 80-86, 138-147, 154-159, 206-217, 226-232, 240-248, 250-257, 259-265, 280-294, 299-326, 328-334, 340-355, 393-398, 415-422, 440-447, 452-458, and/or 375-452 of SEQ ID NO: 437; 4-12, 20-31, 43-49, 100-118, 121-138, 141-148, 153-161, 167-177, 206-213, 225-231, 235-240, 256-267, 277-287, 301-322, 325-333, 336-360, 381-388, 400-415, 447-452, 459-472, and/or 55-75 of SEQ ID NO: 438; 4-10, 29-56, 93-99, 119-124, 133-140, 159-171, 187-195, 200-214, 221-232, 249-255, 263-271, 285-291, 310-316, and/or61-198 of SEQ ID NO: 439; 9-15, 48-65, 72-79, 87-102, 104-115, 118-124, 126-138, 153-185, 188-207, 212-239, 257-265, 297-304, 306-313, and/or 1-54 of SEQ ID NO: 440; 18-38, 48-62, 68-107, 143-158, 167-181, 193-198, 205-213, 220-231, 239-245, 258-264, 279-300, 308-314, 318-328, 343-354, 360-367, 425-433, 465-474, 496-505, 508-514, 529-536, 545-562, 572-580, 587-593, 595-609, 612-618, 627-637, 642-649, 652-673, 688-693, 696-701, 707-736, 748-754, 766-776, 779-786, 791-797, 815-825, 830-842, 857-865, 868-876, 880-887, 898-905, 911-923, 925-936, 961-983, 1011-1018, 1043-1059, 1073-1079, 1093-1104, 1110-1116, 1135-1144, 1146-1163, 1183-1189, 1196-1204, 1222-1242, 1250-1262, 1275-1296, 1322-1330, and/or 1056-1199 of SEQ ID NO: 441; 15-27, 35-40, 47-55, 57-73, 77-93, 103-112, 126-138, 141-179, 192-218, 224-237, 244-257, 263-278, and/or 83-115 of SEQ ID NO: 442; 4-22, 24-29, 36-43, 63-75, 90-96, 118-128, 137-145, 168-182, 198-210, 212-221, 242-250, 289-316, 318-323, 327-339, 381-387, 401-411, 424-434, 443-449, 453-465, 485-498, 500-508, 510-515, 521-528, 538-545, 554-560, 574-606, 619-627, 645-658, 681-688, 70-79, and/or 473-516 of SEQ ID NO: 443; 8-18, 45-50, 52-62, 76-82, 84-107, 109-116, 130-137, 141-150, 152-158, 164-170, 175-186, 188-196, and/or 9-73 of SEQ ID NO: 444; 4-24, 39-46, 84-95, 133-151, 166-174, 179-189, 196-203, 212-218, 223-236, 240-246, 255-261, 266-275, 286-293, 299-316, 323-331, 347-358, 368-380, 382-389, 391-398, 410-417, 420-429, 437-445, 451-456, 464-471, 504-514, 523-532, 539-545, 553-567, 572-588, 594-603, 620-630, 636-641, 656-663, 665-672, 676-687, 693-700, 40-166, and/or 577-605 of SEQ ID NO: 445; 4-42, 48-59, 74-90, 92-119, 121-149, 163-180, 185-192, 199-209, and/or 1-9 of SEQ ID NO: 446; 5-26, 60-76, 104-114, 119-128, 136-141, 156-167, 186-198, 218-237, 260-267, 275-290, 328-335, and/or 294-334 of SEQ ID NO: 447; 4-10, 14-37, 40-47, 68-77, 87-95, 103-111, 116-153, 170-237, 245-251, 253-274, 280-299, 311-318, 321-338, 364-371, 378-392, 395-430, 438-451, 458-475, 479-507, 520-526, 542-560, 573-586, 591-598, 608-614, 636-668, 678-690, 692-698, 702-717, 724-731, and/or 302-448 of SEQ ID NO: 448; 5-17, 23-48, 60-73, 75-82, 98-108, 110-128, 146-160, 168-180, 191-213, 229-237, 240-252, 269-277, 305-313, and/or 63-147 of SEQ ID NO: 449; 4-10, 19-35, 41-47, 51-60, 70-80, 91-115, 170-191, 194-211, 226-232, 234-241, 256-273, 289-294, 311-349, 358-363, 392-400, 406-416, and/or 356-391 of SEQ ID NO: 450; 5-25, 50-57, 67-75, 78-86, 94-112, 122-145, 152-165, 171-183, 193-199, 217-235, 238-253, 255-264, 281-287, 294-303, 309-314, 319-324, 327-341, 349-355, 364-382, 384-392, 397-411, 419-427, 435-452, 455-463, 488-504, 536-541, 558-563, 568-574, 595-601, 614-620, 637-644, and/or 7-127 of SEQ ID NO: 451; 10-16, 39-45, 62-91, 102-114, 120-127, 136-147, 152-159, 163-173, 178-188, 196-217, 223-231, 234-254, 257-267, 270-283, 290-300, 306-312, and/or 224-295 of SEQ ID NO: 452; 4-9, 12-21, 23-52, 54-65, 74-83, 103-117, 131-141, 144-163, 171-177, 183-189, 205-212, 252-262, 297-304, 308-314, 320-329, 343-349, 357-370, 375-380, 395-405, 434-441, 456-465, 474-484, 505-514, 528-536, 540-549, 576-582, 597-607, 616-622, 634-641, 648-654, 690-713, 715-724, 743-751, 757-763, 772-789, 791-802, 809-814, 828-837, 840-846, 853-875, and/or 283-379 of SEQ ID NO: 453; 4-27, 40-46, 55-62, 84-100, 108-114, 118-123, 132-145, 165-171, 178-183, 192-223, 226-232, 234-243, 276-282, 299-305, 326-334, 340-351, 357-371, 374-387, 395-406, 417-437, 443-452, 470-478, 485-494, 496-503, 508-521, 527-537, 541-546, and/or 252-272 of SEQ ID NO: 454; 4-36, 45-50, 58-63, 69-80, 89-97, 99-109, 111-118, 126-132, 141-147, 172-184, 188-197, 208-215, 220-231, 236-241, 253-264, 271-280, 288-297, 342-347, 361-367, 375-382, 388-394, 401-406, 408-414, 441-447, 452-458, 466-476, 483-491, 503-510, 521-528, 539-545, 547-558, 566-576, 584-589, 606-617, 624-636, and/or 418-432 of SEQ ID NO: 455; 7-14, 5-32, 6-72, 95-100, 108-114, 123-135, 143-153, 203-221, 224-230, 260-269, 290-297, 302-308, 320-328, 333-339, and/or 149-248 of SEQ ID NO: 456; 21-27, 30-48, 55-65, 70-90, 97-107, 122-128, 135-166, 172-180, 184-199, 205-224, 237-247, 252-269, 278-283, and/or 240-257 of SEQ ID NO: 457; 4-14, 20-33, 36-43, 49-60, 72-114, 117-123, 125-132, 138-143, 157-175, 184-204, 208-217, and/or 58-89 of SEQ ID NO: 458; 4-15, 23-36, 38-47, 54-64, 92-103, 117-126, 135-155, 157-200, 202-223, 231-239, 246-261, and/or 70-162 of SEQ ID NO: 459; 4-10, 35-45, 56-92, 108-114, 127-146, 160-186, 194-203, 225-234, 242-251, 272-283, 285-306, 310-315, 322-330, 358-371, 373-382, 389-396, and/or 93-188 of SEQ ID NO: 460; 18-32, 35-82, 85-115, 119-142, 149-172, and/or 4-36 of SEQ ID NO: 461; 6-14, 20-28, 35-55, 64-87, 100-109, 144-149, 189-208, 210-218, 221-227, 242-247, 254-264, 283-297, 301-308, 310-322, 351-358, 372-378, 383-389, 421-432, 447-460, 537-545, 550-558, 581-593, 595-606, 645-658, 677-688, and/or 414-503 of SEQ ID NO: 462; 9-34, 47-63, and/or 37-51 of SEQ ID NO: 463; 6-22, 50-58, 73-101, 119-128, 139-154, 167-173, 209-217, 227-234, and/or 1-126 of SEQ ID NO: 464; 10-34, 37-44, 72-78, 87-100, 111-117, 122-143, 177-196, 207-229, 232-249, 255-261, 268-278, 289-300, 315-349, 351-358, 371-378, 386-394, 404-466, 468-480, and/or 341-443 of SEQ ID NO: 465; 16-22, 30-35, 45-52, 54-64, 74-84, 90-98, 120-125, 135-148, 156-162, 166-186, 190-199, 201-215, 226-232, 262-270, 281-289, 322-331, 335-340, 367-372, 379-385, 415-426, 438-445, 455-466, 472-479, 492-497, and/or 341-484 of SEQ ID NO: 466; 4-31, 33-66, 77-83, 90-101, 118-135, 161-166, 168-189, 203-209, 216-222, 231-236, 269-277, 279-290, 298-310, 341-347, 371-376, 380-387, 389-395, 410-419, 446-455, 465-471, 476-484, 513-522, 532-537, 545-556, 560-585, 603-610, 630-657, 660-672, 694-704, 717-728, 738-745, 754-766, 782-792, 794-811, 813-819, 833-853, 901-906, 912-917, 951-979, 985-991, 998-1004, 1013-1019, 1052-1065, 1080-1086, 1124-1130, 1142-1150, 1168-1176, 1182-1193, 1209-1218, 1234-1245, 1271-1277, 1284-1298, 1301-1308, 1339-1345, and/or 529-629 of SEQ ID NO: 467; 21-28, 57-71, and/or 15-46 of SEQ ID NO: 468; 63-99, 101-109, 111-137, 143-172, 175-200, and/or 11-48 of SEQ ID NO: 469; 18-32, 35-82, 85-115, 119-142, 149-172, and/or 6-39 of SEQ ID NO: 470; 67-74, 88-94, 112-118, 127-138, 155-169, 171-180, 183-190, 196-205, 243-249, 260-271, 308-344, 346-373, 381-414, 416-457, 473-513, 515-524, 528-535, 539-544, 556-566, 572-580, 585-590, and/or 26-129 of SEQ ID NO: 471; 17-40, 47-66, 70-78, and/or 29-102 of SEQ ID NO: 472; 4-9, 23-41, 44-51, 58-64, 70-86, 94-111, and/or 47-77 of SEQ ID NO: 473; 4-22, 29-48, 50-58, 62-69, 71-78, and/or 6-35 of SEQ ID NO: 474; 8-19, 31-47, 49-58, 64-79, 84-96, and/or 7-52 of SEQ ID NO: 475; 4-17, 19-26, 41-49, 63-87, 92-99, 113-131, and/or 9-118 of SEQ ID NO: 476; 10-37, 55-68, 71-78, 92-98, 115-122, 131-138, 149-158, 163-170, 172-189, 212-219, 239-257, 259-271, 289-302, 304-320, 322-340, 359-366, 373-384, 400-412, 444-453, 460-474, 485-527, and/or 186-224 of SEQ ID NO: 477; 6-13 of SEQ ID NO: 478; 5-12 of SEQ ID NO: 480; 6-14 of SEQ ID NO: 481; 4-10 of SEQ ID NO: 483; 4-19 of SEQ ID NO: 486; 9-14 of SEQ ID NO: 487; 4-44, 47-60, 65-72, 92-98, and/or 61-95 of SEQ ID NO: 488; 2-17 of SEQ ID NO: 489; 19-29, 39-45, 52-59, and/or 7-32 of SEQ ID NO: 490; 6-20, 36-44, and/or 78-97 of SEQ ID NO: 491; 5-12, and/or 1-22 of SEQ ID NO: 492; 17-24, 26-41, 50-55, and/or 8-32 of SEQ ID NO: 493; 7-18, and/or 13-22 of SEQ ID NO: 494; 24-32, and/or 3-28 of SEQ ID NO: 495; 6-11, 14-35, 40-56, and/or 17-35 of SEQ ID NO: 496; 4-12, 17-23, 42-53, 69-81, and/or 45-64 of SEQ ID NO: 497; 4-25, 28-34, 37-43, 59-69, 104-114, and/or 52-117 of SEQ ID NO: 498; 4-12, and/or 4-22 of SEQ ID NO: 499; 4-9, and/or 1-19 of SEQ ID NO: 500; 4-22, and/or 2-19 of SEQ ID NO: 501; 13-36, 48-63, 80-101, 141-149, 165-176, 184-198, and/or 28-54 of SEQ ID NO: 502; 4-14, 21-26, and/or 21-29 of SEQ ID NO: 503; 4-26, and/or 20-36 of SEQ ID NO: 504; 4-13, and/or 7-22 of SEQ ID NO: 505; 7-13, 21-34, and/or 26-54 of SEQ ID NO: 506; 4-21, 25-44, 59-68, and/or 1-92 of SEQ ID NO: 507; 4-14, 26-33, and/or 12-33 of SEQ ID NO: 508; 12-31, 46-60, 87-94, and/or 1-98 of SEQ ID NO: 509; 5-14, and/or 4-22 of SEQ ID NO: 510; 6-15, 34-52, and/or 14-36 of SEQ ID NO: 511; 5-18, 27-33, 40-48, and/or 17-39 of SEQ ID NO: 512; 8-17, 63-70, 89-100, and/or 21-52 of SEQ ID NO: 513; 4-18, 21-31, 51-68, and/or 34-66 of SEQ ID NO: 514; 4-9, 14-19, and/or 3-31 of SEQ ID NO: 515; 4-10, 21-30, 37-51, and/or 11-44 of SEQ ID NO: 516; 4-9, 32-38, and/or 7-22 of SEQ ID NO: 517; 4-22, 29-36, 38-47, and/or 12-43 of SEQ ID NO: 518; 4-12, 14-23, and/or 26-37 of SEQ ID NO: 519; 4-11, 20-46, and/or 32-59 of SEQ ID NO: 520; 4-10, and/or 1-30 of SEQ ID NO: 521; 4-28, 32-50, and/or 17-51 of SEQ ID NO: 522; 14-28, and/or 2-14 of SEQ ID NO: 523; 4-25, and/or 12-40 of SEQ ID NO: 524; 33-41, 43-53, and/or 16-39 of SEQ ID NO: 525; 9-16, and/or 7-18 of SEQ ID NO: 526; 4-11, 24-31, and/or 18-34 of SEQ ID NO: 527; 14-20, 48-56, 62-69, 81-87, and/or 4-56 of SEQ ID NO: 528; 8-16, and/or 13-39 of SEQ ID NO: 529; 11-16, 26-40, 50-63, and/or 6-48 of SEQ ID NO: 530; 23-34, 59-72, and/or 1-28 of SEQ ID NO: 531; 11-16, 26-82, 95-101, 103-113, 120-164, 179-185, 187-194, 224-248, 255-263, 276-293, 296-301, 304-312, 314-320, 351-374, 390-398, 401-407, 420-426, 434-444, 454-475, 481-508, 521-531, 535-549, and/or 46-77 of SEQ ID NO: 532; 6-30, 32-53, 69-76, 78-86, 96-112, 121-135, 142-175, 177-199, 201-255, 263-269, 277-288, 290-303, 307-345, 353-364, 388-403, 443-455, 462-474, 480-485, and/or 6-30 of SEQ ID NO: 533; 12-61, and/or 14-46 of SEQ ID NO: 534; 4-12, 24-33, 39-51, 57-63, 78-87, and/or 26-51 of SEQ ID NO: 535; 8-15, 29-40, 48-54, and/or 33-56 of SEQ ID NO: 536; 22-31, 59-69, and/or 70-100 of SEQ ID NO: 537; 12-61, and/or 12-43 of SEQ ID NO: 538; 4-16, 28-39, 62-71, 85-97, and/or 54-85 of SEQ ID NO: 539; 10-20, 23-31, 35-42, 48-62, and/or 30-53 of SEQ ID NO: 540; 9-41, 46-52, 70-85, and/or 63-89 of SEQ ID NO: 541; 18-34, 36-46, 71-83, 95-101, 130-143, 149-161, and/or 96-115 of SEQ ID NO: 542; 4-18, 26-66, 68-95, 100-110, 120-135, 143-165, 168-175, 177-198, and/or 160-176 of SEQ ID NO: 543; 4-16, 21-34, 51-56, and/or 10-29 of SEQ ID NO: 544; 4-23, 29-37, 40-70, 78-91, 98-111, and/or 89-115 of SEQ ID NO: 545; 4-34, 56-62, and/or 36-54 of SEQ ID NO: 546; 4-9, and/or 1-29 of SEQ ID NO: 547; 10-22, 24-37, 52-57, 74-81, and/or 16-69 of SEQ ID NO: 548; 4-16, 42-70, 78-93, 95-101, 103-111, and/or 64-88 of SEQ ID NO: 549; 4-9, 22-48, 51-59, 64-94, 100-124, 130-135, and/or 123-134 of SEQ ID NO: 550; 10-20, 27-40, 48-57, and/or 15-58 of SEQ ID NO: 551; 24-37, and/or 12-30 of SEQ ID NO: 552; 30-42, 51-56, 67-76, 79-96, and/or 22-61 of SEQ ID NO: 553; 11-22, 28-35, and/or 18-33 of SEQ ID NO: 554; 4-9, 13-21, 37-42, and/or 23-36 of SEQ ID NO: 555; 4-12, and/or 12-22 of SEQ ID NO: 556; 39-50, 71-89, 95-106, 126-139, 154-162, and/or 58-143 of SEQ ID NO: 557; 5-26, 38-46, 54-62, 69-81, 87-99, 103-117, 120-136, 138-161, 168-189, 201-207, and/or 136-167 of SEQ ID NO: 558; 4-21, 33-39, 41-55, 61-68, 73-98, 104-110, 121-127, 131-156, and/or 97-115 of SEQ ID NO: 559; 9-26, 36-48, and/or 21-40 of SEQ ID NO: 560; 4-12, 26-32, and/or 12-24 of SEQ ID NO: 561; 4-12, 17-23, 39-62, and/or 8-34 of SEQ ID NO: 562; 17-41, 47-66, and/or 12-30 of SEQ ID NO: 563; 4-11, 15-25, 33-52, and/or 1-27 of SEQ ID NO: 564; 4-11, 33-40, 48-76, 96-104, and/or 84-106 of SEQ ID NO: 565; 15-22, and/or 11-26 of SEQ ID NO: 566; 8-19, 44-53, 61-71, 78-85, 97-107, and/or 49-89 of SEQ ID NO: 567; 13-23, 31-44, 59-66, 84-90, 96-110, and/or 47-147 of SEQ ID NO: 568; 4-24, 56-73, 83-97, 112-132, 140-150, 161-184, and/or 111-146 of SEQ ID NO: 569; 4-17, 19-26, 41-48, 63-87, 92-99, 113-131, and/or 10-136 of SEQ ID NO: 570; 4-11, 17-26, and/or 6-32 of SEQ ID NO: 571; 26-38, 48-55, 72-86, 90-107, 123-132, 134-161, 181-187, and/or 3-19 of SEQ ID NO: 572; 4-19, 26-35, and/or 11-46 of SEQ ID NO: 573; 5-10, 21-38, 42-70, 84-103, and/or 92-127 of SEQ ID NO: 574; 6-17, 34-40, 42-51, 75-85, 94-100, and/or 38-63 of SEQ ID NO: 575; 6-13, 21-27, 29-35, 57-64, and/or 32-63 of SEQ ID NO: 576; 4-19, 22-48, 54-62, 73-91, 94-109, and/or 75-98 of SEQ ID NO: 577; 9-25, and/or 12-28 of SEQ ID NO: 578; 16-21, and/or 8-25 of SEQ ID NO: 579; 4-24, 26-32, 45-55, 58-67, 76-93, 104-111, 117-122, 128-136, and/or 33-119 of SEQ ID NO: 580; -11, 31-37, 56-63, 66-84, and/or 32-58 of SEQ ID NO: 581; 6-69, 75-87, 89-111, 149-156, and/or 52-139 of SEQ ID NO: 582; 4-13, 19-29, 49-68, and/or 24-49 of SEQ ID NO: 583; 19-25, 27-33, 43-84, 86-92, 111-118, 125-136, 138-147, and/or 95-124 of SEQ ID NO: 584; 20-29, 50-56, 63-85, 89-98, 110-128, and/or 2-29 of SEQ ID NO: 585; 4-11, 41-47, 62-71, and/or 7-34 of SEQ ID NO: 586; 23-30, 48-57, 67-72, 81-89, and/or 11-31 of SEQ ID NO: 587; 14-27, 50-62, and/or 22-57 of SEQ ID NO: 588; 4-17, 23-37, and/or 13-33 of SEQ ID NO: 589; 5-33, 38-57, 60-68, and/or 18-37 of SEQ ID NO: 590; 4-19, 44-51, and/or 24-45 of SEQ ID NO: 591; 17-40, 47-66, 70-78, and/or 67-101 of SEQ ID NO: 592; 4-20, 24-29, 43-55, 57-63, 74-83, 137-157, 171-179, 181-192, 237-243, 273-279, 300-310, 312-321, 324-330, 366-380, 401-417, and/or 61-120 of SEQ ID NO: 593; 6-21, and/or 17-43 of SEQ ID NO: 594; 11-17, and/or 9-31 of SEQ ID NO: 595; 18-28, 38-44, 53-59, 64-74, 78-85, and/or 10-96 of SEQ ID NO: 596; 12-40, 49-58, and/or 5-49 of SEQ ID NO: 597; 28-34, 46-52, 54-68, 72-85, 95-104, and/or 66-94 of SEQ ID NO: 598; 4-14, and/or 1-25 of SEQ ID NO: 599; 4-18, 30-37, 51-65, 83-89, 99-105, 108-136, and/or 24-98 of SEQ ID NO: 600; 26-34, 36-44, 68-78, 85-92, 96-101, 127-134, 141-148, 156-166, 186-192, 244-256, 281-287, 291-301, 308-316, 321-343, 368-382, 385-391, 394-404, 414-429, 453-465, 471-489, and/or 147-240 of SEQ ID NO: 997; 10-18, 26-48, 58-68, 72-78, 94-105, 115-130, 155-164, 170-177, 179-185, 201-219, 243-260, 267-277, 295-302, 350-376, 398-403, 429-437, 451-462, 471-478, 504-512, 563-568, 570-583, 589-594, 614-629, 1-123, and/or 161-546 of SEQ ID NO: 998; 10-20, 35-45, 59-93, 163-168, 190-196, 200-210, 233-261, 270-279, 306-329, 331-353, 363-381, 388-394, 399-425, 443-460, 462-474, 477-498, 506-516, 522-542, 546-559, 570-577, 100-301, and/or 446-518 of SEQ ID NO: 999; 4-13, 28-34, 52-71, 78-90, 118-140, 147-156, 167-187, 264-275, 286-292, 305-310, 328-334, 340-346, 351-362, 31-117, and/or 165-354 of SEQ ID NO: 1000; 38-45, 76-84, 91-103, 111-118, 147-162, 166-177, 187-201, 208-215, 242-249, 267-274, 295-301, 309-322, 18-100, and/or 111-296 of SEQ ID NO: 1001; 4-9, 25-39, 41-47, 70-78, 82-103, 106-140, 152-188, 192-198, 200-207, 211-217, 232-251, 271-277, 285-299, 307-314, 323-335, 344-370, 376-382, 388-398, 417-422, 428-446, 448-456, 462-468, 494-504, 526-533, 565-573, 199-383, and/or 454-579 of SEQ ID NO: 1002; 10-27, 50-87, 94-109, 115-128, 134-152, 155-169, 175-193, 196-213, 216-233, 235-251, 259-270, 272-283, 293-303, 311-318, 327-339, 348-374, 380-386, 392-402, 421-426, 432-450, 452-460, 466-472, 498-508, 530-537, and/or 206-363 of SEQ ID NO: 1003; 9-16, 25-31, 48-86, 90-101, 109-127, 131-147, 150-187, 189-195, 204-211, 213-226, 237-248, 250-261, 271-281, 289-296, 305-317, 326-352, 358-364, 370-380, 399-404, 410-428, 430-438, 444-450, 476-486, 508-515, 547-555, 1-118, 208-283, and/or 494-564 of SEQ ID NO: 1004; 31-37, 57-66, 73-97, 99-117, 119-137, 141-149, 157-172, 179-185, 190-196, 203-212, 216-224, 226-238, 240-251, 261-271, 279-286, 295-307, 316-342, 348-354, 360-370, 389-394, 400-418, 420-428, 434-440, 466-476, 498-505, 537-545, 1-98, 233-351, and/or 423-538 of SEQ ID NO: 1005; 27-37, 44-58, 66-80, and/or 5-70 of SEQ ID NO: 1006; 4-32, 35-69, 92-98, 113-137, 168-190, 224-235, 269-277, 279-289, 322-329, 370-376, 380-387, 389-395, 411-420, 442-457, 463-473, 476-487, 510-521, 531-554, 561-567, 570-585, 604-610, 612-624, 632-651, 660-672, 690-702, 718-730, 738-745, 782-809, 813-820, 823-829, 844-854, 901-906, 911-917, 959-967, 985-991, 997-1006, 1010-1019, 1036-1045, 1053-1059, 1079-1086, 1124-1132, 1137-1150, 1168-1180, 1182-1193, 1209-1214, 1216-1226, 1231-1245, 1271-1277, 1290-1298, 1301-1307, 1339-1345, 22-86, 138-260, and/or 490-641 of SEQ ID NO: 1007; 37-43, 50-57, 65-82, 86-109, 123-129, 141-150, 152-157, 166-172, 179-203, 209-241, 249-296, 298-307, 312-326, 329-335, 341-348, 364-377, 379-399, 401-409, 411-417, 420-425, 438-444, 461-466, 473-480, 497-505, 522-534, 541-550, 586-597, 608-614, 622-632, 660-666, 679-694, 697-706, 708-731, 737-772, 784-789, 810-825, 837-873, 882-895, 901-928, and/or 214-233 of SEQ ID NO: 1008; 10-16, 18-27, 41-61, 81-90, 133-142, and/or 43-118 of SEQ ID NO: 1009; 21-47, 50-69, 83-89, 113-120, 129-146, 152-158, 160-189, 204-225, 235-249, 251-263, 265-275, and/or 107-140 of SEQ ID NO: 1010; 7-12, 16-24, 28-46, 61-68, 79-85, 87-93, 95-102, 108-123, 148-164, 166-172, 177-202, 205-215, 231-246, 254-261, 267-273, 280-294, 309-315, 322-329, 337-342, 345-351, 386-394, 406-413, 449-455, 473-480, 490-497, 501-508, 532-539, 576-583, 623-629, 657-665, 681-708, 715-722, 751-757, and/or 584-613 of SEQ ID NO: 1011; 10-26, 52-88, 94-109, 115-172, 175-189, 196-210, 215-232, 244-252, 260-270, 272-283, 293-303, 311-318, 327-339, 348-374, 380-386, 392-402, 421-426, 432-450, 452-460, 466-472, 498-508, 530-537, 569-577, and/or 258-365 of SEQ ID NO: 1012; 25-46, 59-65, 68-75, 83-90, 93-100, 107-115, 124-135, 151-177, 183-189, 194-206, 209-215, 219-224, 251-263, 267-276, 305-311, 318-327, 332-338, 350-356, 380-396, 406-412, 414-423, 431-437, 453-461, 463-481, 483-491, 505-510, 513-523, 528-545, 568-575, and/or 245-271 of SEQ ID NO: 1013; 4-19, 21-29, 61-68, 87-95, 103-113, 145-154, 157-170, and/or 10-76 of SEQ ID NO: 1014; 4-18, 28-36, 41-65, 69-84, 96-103, 106-115, 118-124, 126-132, 148-156, 169-181, 187-194, 221-227, and/or 1-63 of SEQ ID NO: 1015; 5-33, 67-78, 122-129, 141-150, 172-185, 201-209, 217-223, 235-252, 303-316, 355-368, 383-389, 400-406, 411-420, 426-437, 445-451, 459-467, 479-496, 512-517, 523-530, 535-562, 577-584, 590-605, 610-632, 644-654, 663-669, 680-686, and/or 309-334 of SEQ ID NO: 1016; 31-38, 73-84, 97-121, 124-135, 141-146, 156-170, 174-191, 205-210, and/or 85-153 of SEQ ID NO: 1017; 24-30, 49-69, 71-111, 113-127, 133-145, 153-169, 172-203, 220-236, 247-254, 256-267, 277-287, 295-302, 311-323, 332-345, 348-358, 364-370, 376-386, 405-410, 416-434, 436-444, 450-456, 482-492, 514-521, 548-561, and/or 243-286 of SEQ ID NO: 1018; 4-10, 22-31, 39-53, 85-94, 102-110, 130-137, 143-149, 154-161, 174-179, 184-190, 197-203, 206-212, 220-227, 240-248, 251-265, 267-286, 297-310, 338-357, 365-370, 373-382, and/or 260-345 of SEQ ID NO: 1019; 9-48, 54-71, 84-92, 114-145, 160-188, 190-200, 216-236, 243-255, 261-268, 281-304, 315-322, 330-336, 342-351, 370-380, 383-393, 400-408, 412-420, 436-442, 452-459, 473-480, 501-512, 518-525, 532-541, and/or 1-94 of SEQ ID NO: 1020; 50-59, 66-81, 90-126, 129-185, 187-205, 213-219, 228-240, 254-266, 268-279, 289-299, 307-314, 323-335, 344-370, 376-382, 388-398, 417-422, 428-446, 448-456, 462-468, 494-504, 526-533, 565-573, and/or 275-374 of SEQ ID NO: 1021; 5-14, 61-74, 91-99, 110-116, 119-136, 138-149, 159-169, 188-194, 205-227, 236-244, 249-256, 294-305, 321-342, 350-364, 373-378, 385-392, 404-420, 422-430, 462-470, 491-500, 513-520, 583-591, 617-638, 652-663, 674-680, 684-698, 709-718, 745-753, 757-763, 781-788, 835-841, 844-854, 861-869, 882-890, 898-916, 934-940, 946-952, 979-990, and/or 443-534 of SEQ ID NO: 1022; 23-35, 71-77, 94-100, 134-140, 157-163, 188-212, 214-221, 262-272, 287-293, 323-331, 360-372, 374-380, 402-411, 421-429, 438-443, 462-473, 477-486, 523-528, 530-547, 57-155, and/or 324-404 of SEQ ID NO: 1023; 11-36, 43-59, 62-70, 78-90, 159-165, 176-188, 197-202, 206-213, 220-225, 234-251, 258-275, 286-293, 300-311, 329-337, 352-361, 363-369, 383-396, 399-417, 420-432, 451-466, 472-478, 504-526, 546-552, 558-566, 576-583, 591-609, 621-628, 642-650, 657-663, 676-681, 692-706, 713-724, 734-753, 763-778, 787-807, 819-825, 57-82, and/or 445-461 of SEQ ID NO: 1024; 14-26, 38-46, 50-57, 76-87, 89-104, 107-112, 123-134, 136-142, 148-162, 173-194, 200-206, 208-216, 226-233, 243-256, 264-307, 338-343, 351-359, 366-376, and/or 133-157 of SEQ ID NO: 1025; 13-19, 30-37, 46-60, 75-82, 100-105, 109-115, 134-140, 146-161, 186-192, 199-205, 207-215, 223-230, 254-260, 281-289, 297-311, 344-352, and/or 251-352 of SEQ ID NO: 1026; 5-16, 19-24, 43-54, 56-89, 104-122, 126-139, 144-157, 165-202, 232-244, 252-265, 272-277, 281-287, 289-300, 308-320, 344-355, 364-390, 396-402, 408-418, 437-442, 448-466, 468-476, 482-488, 514-524, 546-553, 585-593, 284-400, and/or 486-600 of SEQ ID NO: 1027; 21-27, 29-46, 50-58, 60-70, 76-98, 100-110, 116-122, 124-130, 145-166, 170-188, 199-209, 214-221, 229-236, 244-259, 270-305, 308-314, 319-329, 348-355, 376-383, 396-442, 446-456, 461-466, 479-485, 513-524, 528-533, 539-546, 556-563, 574-585, 595-602, 604-616, 618-625, 630-647, 649-656, 662-674, 680-686, 689-700, 716-739, and/or 586-630 of SEQ ID NO: 1028; 4-19, 31-49, 82-88, 92-98, 111-141, 146-153, 161-172, 174-197, 199-214, 218-226, 233-239, 242-250, 256-266, 279-293, 298-309, 321-331, 338-345, and/or 223-248 of SEQ ID NO: 1029; 24-41, 54-61, 63-77, 83-91, 100-111, 116-121, 128-133, 139-146, 153-164, 166-176, 212-242, 255-266, 269-275, 277-285, 290-299, and/or 144-277 of SEQ ID NO: 1030; 4-29, 33-47, 93-109, 117-125, 152-168, 175-180, 213-219, 224-234, 261-267, 270-284, 321-326, 328-342, 359-365, 383-389, 401-407, 417-424, 471-478, 491-497, 532-537, 545-555, 569-583, 646-652, 688-694, 711-718, 735-745, 794-803, 813-823, 834-839, 851-857, 860-867, 874-882, 895-900, 902-910, 917-923, 933-940, 948-955, 960-966, 1001-1007, 1045-1053, 1058-1066, 1087-1101, 1103-1114, 1130-1139, 1141-1149, 1155-1166, 1192-1198, 1211-1219, and/or 549-657 of SEQ ID NO: 1031; 13-48, 57-63, 73-81, 89-103, 107-114, 119-125, 130-140, 146-167, 175-186, 202-210, 212-220, 233-239, 255-270, 272-286, 288-301, and/or 10-23 of SEQ ID NO: 1032; 5-41, 48-56, 75-81, 86-93, 100-111, 126-134, 150-156, 168-188, 209-236, 250-257, 260-274, 291-305, 311-335, 337-344, 373-379, 393-406, 423-450, 461-467, and/or 105-135 of SEQ ID NO: 1033; 8-16, 37-88, 105-117, 141-169, 172-178, 189-197, 210-217, 243-249, 253-260, 269-282, 293-299, 315-332, 363-373, 378-401, 406-415, 417-423, 454-460, 465-471, 503-510, 515-521, 538-576, 578-616, 619-625, 636-643, 651-669, 671-686, 691-698, 719-726, 734-748, 762-778, 782-795, 799-810, 821-845, 894-908, 917-925, 937-943, 946-967, 969-975, and/or 444-479 of SEQ ID NO: 1034; 31-45, 47-55, 68-82, 87-100, 103-108, 112-117, 134-140, 157-163, 166-175, 178-185, 197-206, 213-219, 234-244, 265-272, 280-289, 292-305, 312-317, 322-328, 335-344, 349-356, 361-372, 376-383, 399-421, 426-444, 455-475, 490-495, and/or 1-84 of SEQ ID NO: 1035; 4-10, 43-53, 64-100, 116-123, 135-141, 145-154, 164-194, 202-211, 233-242, 250-259, 280-291, 293-314, 318-323, 330-338, 366-379, 381-387, 397-404, and/or 70-188 of SEQ ID NO: 1036; 4-13, 20-28, 33-49, 61-70, 76-94, 100-146, 151-163, 175-182, 202-212, 218-225, 241-252, and/or 12-53 of SEQ ID NO: 1037; 12-46, 49-64, 81-97, 103-108, 119-140, 150-173, 179-193, 196-213, 215-223, 225-235, 238-253, 259-264, 267-274, 278-284, 292-305, 314-321, 340-346, and/or 26-49 of SEQ ID NO: 1038; 18-23, 28-50, 100-150, 157-186, 197-203, 219-231, 233-240, 257-280, and/or 10-45 of SEQ ID NO: 1039; 10-16, 29-49, 59-73, 79-118, 154-169, 178-192, 204-209, 216-224, 231-242, 250-256, 269-275, 290-311, 319-325, 329-339, 354-365, 371-378, 436-444, 476-485, 507-516, 519-525, 540-547, 556-573, 583-591, 598-604, 606-620, 623-629, 638-648, 653-660, 663-684, 699-704, 707-712, 718-747, 759-765, 777-787, 790-797, 802-808, 826-836, 841-853, 868-876, 879-887, 891-898, 909-916, 922-934, 936-947, 972-994, 1022-1029, 1054-1070, 1084-1090, 1104-1115, 1121-1127, 1146-1155, 1157-1174, 1194-1200, 1207-1215, 1233-1253, 1261-1273, 1286-1307, 1333-1341, and/or 1054-1156 of SEQ ID NO: 1040; 14-26, 28-37, 40-49, 58-78, 89-95, 107-118, 125-132, 135-142, 155-161, 175-185, 195-213, 215-225, 233-241, 248-254, 263-271, 294-323, 334-340, 345-351, 355-368, 371-394, 401-410, 419-424, 449-456, 463-469, 483-496, and/or 113-187 of SEQ ID NO: 1041; 10-35, 52-116, 133-143, 149-170, 176-184, and/or 94-134 of SEQ ID NO: 1042; 4-10, 13-19, 31-39, 57-72, 92-103, 109-122, 126-144, 168-174, 179-192, 234-245, 257-262, 36-62, 157-180, and/or 190-270 of SEQ ID NO: 1043; 4-29, 36-52, 60-68, 70-90, and/or 5-73 of SEQ ID NO: 1044; 5-12, 22-56, 58-63, 69-82, 140-146, 175-180, 204-212, 240-248, 276-283, 307-312, 324-329, 335-353, 372-385, 403-412, 436-443, 448-464, 468-474, 476-483, 503-514, 566-573, 590-597, 601-611, 619-626, 630-639, 647-655, 666-679, 689-697, 707-719, 721-728, 761-769, 783-789, 797-803, 806-812, 845-853, 864-870, 893-904, 917-923, 949-956, 967-985, 1005-1021, 1027-1034, 1059-1065, and/or 1-335 of SEQ ID NO: 1045; 4-16, 18-28, 48-55, 67-75, 81-87, 94-100, 108-124, 139-147, 150-156, and/or 110-136 of SEQ ID NO: 1046; 4-22, 28-37, 39-46, and/or 28-38 of SEQ ID NO: 1047; 4-10, 13-19, 27-34, 40-103, 106-113, and/or 79-100 of SEQ ID NO: 1048; 11-23 of SEQ ID NO: 1049; 4-10, 19-25, 45-51, 64-75, 79-89, and/or 73-96 of SEQ ID NO: 1050; 4-22, 29-36, 38-47, and/or 21-44 of SEQ ID NO: 1051; 8-20, 39-44, and/or 26-42 of SEQ ID NO: 1052; 8-19, 31-47, 49-58, 64-79, 84-96, and/or 16-50 of SEQ ID NO: 1053; 20-26, 31-37, and/or 4-13 of SEQ ID NO: 1054; 4-10, 16-32, 34-42, 60-66, and/or 7-25 of SEQ ID NO: 1055; 4-9, 23-45, and/or 8-44 of SEQ ID NO: 1056; 4-19, 34-40, 53-81, 103-117, 122-187, and/or 22-51 of SEQ ID NO: 1057; 4-24, and/or 1-30 of SEQ ID NO: 1058; 2-27 of SEQ ID NO: 1059; 4-18, and/or 9-44 of SEQ ID NO: 1060; 4-17, 19-26, 41-49, 63-87, 92-99, 113-131, and/or 6-105 of SEQ ID NO: 1061; 6-12, and/or 9-32 of SEQ ID NO: 1062; 4-44, 49-62, and/or 22-65 of SEQ ID NO: 1063; 20-28, 34-40, and/or 1-14 of SEQ ID NO: 1064; 4-29, 35-57, and/or 17-43 of SEQ ID NO: 1065; 14-29, and/or 24-35 of SEQ ID NO: 1066; 4-19, 31-47, 62-73, 76-83, 87-93, 99-106, and/or 10-38 of SEQ ID NO: 1067; 4-10, 12-26, and/or 1-28 of SEQ ID NO: 1068; 16-30, 38-45, 55-61, 69-75, 131-141, 159-165, 169-179, 182-191, 206-214, 230-245, 252-262, 272-280, 299-305, 328-339, 362-370, 372-378, 388-393, 401-409, and/or 148-226 of SEQ ID NO: 1203; 13-18, 24-33, 36-49, 69-79, 93-101, 113-119, 129-136, 139-164, 167-175, 182-193, 195-217, 224-231, 282-293, 336-345, 351-358, 373-378, 382-387, 407-415, 441-447, 451-459, 461-470, 479-485, 499-526, 533-544, 559-569, 572-581, 583-592, 598-609, 613-624, 632-649, 654-660, 666-694, 700-706, 712-722, 738-752, 771-778, 800-816, 824-839, 847-864, 879-885, 918-925, 927-946, 957-967, 971-999, 1006-1014, 1018-1037, 1046-1055, 1057-1071, 1090-1098, 1104-1113, 1115-1128, 1181-1187, 1193-1202, 1205-1211, 1223-1234, 1240-1248, 1259-1265, 1273-1281, 1289-1301, 1336-1343, 1346-1352, 1356-1363, 1365-1378, 1382-1391, 1401-1408, 1416-1423, 1433-1443, 1450-1455, 1461-1467, 1475-1481, 1486-1493, 1-14, and/or 1115-1214 of SEQ ID NO: 1204; 6-11, 31-53, 69-84, 86-93, 102-108, 112-121, 135-145, 157-177, 185-202, 208-226, 232-241, 244-251, 260-289, 306-316, and/or 199-267 of SEQ ID NO: 1205; 4-25, 36-41, 44-49, 68-78, 83-89, 95-102, 113-124, 137-142, 163-175, 179-185, 209-218, 233-244, 250-261, 279-288, 308-322, 330-336, and/or 49-119 of SEQ ID NO: 1206; 4-24, 42-50, 57-75, 101-107, 109-131, 153-161, 180-190, 214-223, 226-240, 248-265, 197-220, and/or 252-267 of SEQ ID NO: 1207; 4-24, 56-62, 111-117, 125-130, 138-143, 153-167, 169-175, 195-204, 207-217, 234-250, 284-298, 303-308, 314-326, 351-356, 359-371, and/or 15-111 of SEQ ID NO: 1208; 4-31, 58-64, 74-80, 88-94, 116-127, 131-138, 141-149, 1-63, and/or 105-163 of SEQ ID NO: 1209; 4-9, 15-26, 33-40, 57-68, 91-108, 112-124, 132-152, 158-168, 186-213, 228-236, 253-269, 276-283, and/or 203-222 of SEQ ID NO: 1210; 4-18, 31-48, 50-61, 81-88, 103-110, 114-121, 143-152, 154-164, 176-194, 199-210, 217-225, 234-240, 245-254, 264-279, and/or 138-153 of SEQ ID NO: 1211; 4-20, 35-41, 51-61, 76-88, 107-115, 122-128, and/or 13-108 of SEQ ID NO: 1212; 4-18, 25-31, 41-46, 61-70, 79-85, 126-133, 135-141, 166-172, 175-181, 191-199, 208-217, 250-258, 261-273, 312-320, 370-376, 385-394, 401-409, 411-416, 431-445, 457-478, 531-543, 560-567, 575-593, 607-615, 625-633, 679-689, 733-740, 746-752, 157-219, and/or 432-508 of SEQ ID NO: 1213; 4-28, 47-53, 58-64, 88-97, 124-129, 136-141, 146-167, 174-184, 187-193, 195-203, 205-220, 231-237, and/or 64-92 of SEQ ID NO: 1214; 7-39, 44-56, 60-65, 83-99, 117-132, 142-148, 162-179, 185-203, 213-231, 239-245, 248-259, 288-295, 330-335, 370-381, 391-396, 401-407, 443-462, 472-492, 505-512, 521-530, 549-557, 562-577, 647-653, 660-666, 673-680, 695-703, 710-717, 729-734, 745-751, 756-766, 769-778, 787-794, 812-819, 825-834, 55-76, and/or 667-736 of SEQ ID NO: 1215; 5-11, 21-27, 48-56, 64-73, 75-85, 110-117, 124-130, 132-138, 145-163, 165-171, 186-198, 231-239, 246-254, 265-270, 289-296, 312-322, and/or 100-124 of SEQ ID NO: 1216; 16-24, 26-63, 66-77, 91-97, 100-111, 148-169, 182-188, 205-212, 223-230, 235-244, 264-272, 291-297, 305-312, and/or 252-296 of SEQ ID NO: 1217; 4-17, 19-31, 34-44, 47-59, 87-93, 99-105, 113-119, 124-137, 139-146, 150-163, 165-175, 185-191, 197-214, 222-227, 235-246, 257-270, 274-279, and/or 85-152 of SEQ ID NO: 1218; 4-20, 40-52, 74-81, 110-117, 123-138, 144-150, 163-181, 185-195, 199-232, 234-248, 269-277, 280-286, 301-314, 324-329, and/or 33-55 of SEQ ID NO: 1219; 4-19, 33-55, 67-74, 78-84, 91-105, 109-117, 132-138, 167-176, 190-196, 202-207, 210-217, and/or 151-162 of SEQ ID NO: 1220; 4-14, 20-26, 28-37, 77-84, 89-94, 114-123, 174-180, 200-208, 222-239, 241-251, 263-271, 276-282, 291-302, 330-335, 374-390, 392-400, 432-441, 447-454, 462-467, 487-501, 516-526, 530-537, 550-566, and/or 77-110 of SEQ ID NO: 1221; 16-23, 25-31, 42-48, 57-75, 81-95, 108-118, 124-151, 153-170, 178-185, 190-201, 216-222, 230-260, 290-297, 300-307, 312-318, 326-334, 348-363, 365-385, and/or 89-142 of SEQ ID NO: 1222; 41-50, 52-70, 80-91, 123-131, 136-143, 146-158, 167-178, 207-213, 228-235, 242-264, 274-288, 302-307, 335-347, 349-355, and/or 59-203 of SEQ ID NO: 1223; 5-14, 21-31, 42-71, 76-103, 111-119, 123-132, 138-144, 167-188, 194-200, 205-212, 230-246, 253-260, 263-269, 275-294, 300-319, 330-343, 349-354, and/or 335-347 of SEQ ID NO: 1224; 6-14, 18-57, 67-81, 87-97, 104-109, 115-122, 125-139, 147-156, 178-188, 193-199, 211-216, 221-235, 265-287, 293-300, 312-323, 330-352, 369-379, 385-390, 392-404, 408-420, 437-457, 471-483, 489-494, 501-509, 525-535, 547-554, and/or 410-444 of SEQ ID NO: 1225; 4-18, 55-82, 87-100, 105-117, 145-150, 168-175, 183-189, 198-203, 228-235, 247-256, 277-283, 293-301, 308-321, 337-362, 373-379, 392-397, 427-434, 457-463, 502-510, 539-552, 560-566, 615-629, 99-226, and/or 566-643 of SEQ ID NO: 1226; 4-27, 59-75, 83-88, 95-105, 112-120, 128-144, 164-169, 200-205, 220-226, 237-242, 253-259, 264-270, 1-103, and/or 124-232 of SEQ ID NO: 1227; 12-42, 69-77, 103-109, 120-128, 149-157, 161-166, 179-197, 201-218, 238-248, 253-261, 266-272, 278-286, 307-314, 138-216, and/or 272-287 of SEQ ID NO: 1228; 4-26, 28-43, 50-56, 62-67, 103-109, 121-130, 155-163, 173-179, 197-202, 208-215, 221-233, 258-267, 296-305, 308-314, 321-328, 41-91, and/or 179-200 of SEQ ID NO: 1229; 4-15, 23-32, 48-55, 61-67, 86-98, 102-111, 124-130, 137-157, 166-171, 178-186, 193-200, 218-232, 236-241, 246-264, 274-288, 291-310, 338-346, 348-359, 389-395, 402-414, 416-428, 430-443, 445-451, 455-469, 473-484, 489-499, 501-509, 511-525, 534-542, 556-561, 579-593, 601-621, 625-637, and/or 475-513 of SEQ ID NO: 1230; 23-30, 33-50, 53-59, 106-114, 126-132, 140-146, 171-180, 196-204, 224-240, 242-254, 262-272, 274-282, 288-296, 326-332, 341-352, 378-387, 394-407, 412-418, 431-464, 489-496, and/or 88-176 of SEQ ID NO: 1231; 4-35, 43-52, 60-79, 93-100, 120-139, 146-154, 157-171, 208-226, 234-247, 265-271, 273-283, 292-298, 309-323, 330-339, 355-382, 396-409, 445-453, 455-465, 484-505, 512-525, 528-535, 547-576, 584-594, and/or 488-559 of SEQ ID NO: 1232; 5-28, 34-41, 47-54, 79-100, 112-117, 122-127, 130-139, and/or 43-96 of SEQ ID NO: 1233; 4-23, 25-38, 45-50, 68-77, 105-113, 149-156, 177-188, 197-208, 222-229, and/or 84-145 of SEQ ID NO: 1234; 11-16, 19-32, 37-43, 62-69, 72-80, 82-93, 97-128, 133-142, 156-166, 179-193, 199-205, 209-216, 239-256, 269-278, 295-325, 332-349, 369-379, 6-26, and/or 347-362 of SEQ ID NO: 1235; 4-10, 17-28, 39-48, 67-82, 93-100, 122-128, 144-153, 177-184, 196-208, 263-285, 289-295, 297-310, 353-359, 367-377, 389-409, 413-421, 426-433, 436-442, 445-451, 453-459, 468-475, 477-488, 506-526, 544-554, 562-611, 639-650, 678-692, 697-706, 718-724, 733-744, 746-754, 766-782, 789-796, 808-822, 831-843, 847-856, 861-868, 887-902, 931-937, 939-951, 957-964, 970-976, 997-1003, 1017-1024, 1033-1039, 1048-1054, 1060-1066, 1075-1083, 1092-1130, 1136-1152, 1168-1176, 1210-1221, 1228-1238, 1242-1247, and/or 934-1001 of SEQ ID NO: 1236; 73-86, 88-95, 113-118, 166-178, 223-230, 243-280, and/or 202-216 of SEQ ID NO: 1237; 16-22, 40-46, 51-62, 77-83, 93-106, 116-124, 127-138, 151-158, 162-177, 185-207, 211-220, 223-234, 261-269, 271-280, 309-317, 339-350, and/or 50-127 of SEQ ID NO: 1238; 11-26, 36-42, 74-85, 88-101, 106-121, 137-156, 186-198, 203-224, 226-254, 295-308, 311-318, 327-355, 373-385, 417-443, 445-464, and/or 95-163 of SEQ ID NO: 1239; 5-11, 16-23, 34-43, 48-56, 71-83, 89-96, 101-110, 138-162, 164-180, 189-195, 204-214, 222-227, 229-250, 255-261, 272-278, and/or 19-90 of SEQ ID NO: 1240; 13-19, 26-53, 67-73, 76-82, 87-102, 112-121, 123-148, 151-156, 162-170, 175-186, 199-209, 211-217, 230-244, 251-287, 300-305, 368-374, 403-415, 418-424, and/or 339-446 of SEQ ID NO: 1241; 4-19, 22-30, 59-73, 84-92, 97-117, 128-140, 165-173, 224-234, 245-255, 276-284, 302-309, 323-330, 349-355, 364-376, 380-385, 391-402, 404-415, 424-432, 437-446, 449-477, 484-499, 516-526, 537-544, 550-555, 562-568, 573-601, 616-623, 653-663, 681-718, 720-728, 762-771, 779-799, 801-806, 814-820, 827-835, 851-870, 899-906, 950-957, 973-990, 995-1005, 1036-1052, 1071-1077, 1086-1098, 1147-1152, 1167-1194, and/or 866-789 of SEQ ID NO: 1242; 27-55, 57-64, 66-75, 135-142, 172-178, 191-197, 229-235, 241-246, and/or 184-217 of SEQ ID NO: 1243; 4-24, 33-39, 55-83, 91-114, 126-132, 137-159, 164-189, 202-224, 232-238, 241-253, 259-282, 285-308, 310-335, 359-364, 371-388, and/or 113-128 of SEQ ID NO: 1244; 6-28, 54-59, 103-114, 122-130, 145-153, 167-175, 184-190, 193-204, 207-212, 215-223, 282-289, 291-300, 315-321, 327-339, 347-354, and/or 10-103 of SEQ ID NO: 1245; 14-37, 52-57, 73-85, 96-113, 124-136, 142-148, 150-157, 166-172, 182-194, 199-215, 217-224, and/or 64-160 of SEQ ID NO: 1246; 4-18, 20-33, 59-77, 93-100, 120-129, 131-137, 140-146, 148-158, 166-172, 185-191, 243-249, 253-258, 295-307, 327-333, 345-370, 387-394, 425-432, 483-489, 491-501, 521-527, 538-553, 560-570, 576-582, 629-637, 649-658, and/or 406-431 of SEQ ID NO: 1247; 9-17, 34-39, 41-59, 71-84, 86-98, 148-169, 177-187, 194-203, 207-214, 222-228, 235-258, 265-273, 286-296, 300-307, 316-328, 338-361, 367-375, 394-401, 407-418, 425-434, 480-495, 502-522, 544-560, 568-575, 584-592, 600-622, 636-641, 661-667, 669-690, 700-707, 719-731, 733-739, 745-750, 767-776, 784-791, 795-811, 840-846, 853-866, 664-745, and/or 793-856 of SEQ ID NO: 1248; 19-54, 86-91, 98-105, 109-124, 126-136, 145-150, 173-178, 196-204, 212-224, 229-239, 246-252, 279-299, 307-313, 323-329, 337-345, 349-361, 382-393, 399-406, 416-421, and/or 404-429 of SEQ ID NO: 1249; 20-26, 66-71, 84-97, 105-111, 122-137, 145-165, 170-185, 204-210, 230-242, and/or 64-158 of SEQ ID NO: 1250; 4-16, 28-69, 72-85, 87-99, 101-123, 128-136, 140-159, 161-173, 185-205, 207-220, 227-240, 242-252, 274-280, 290-296, 301-326, 356-379, 399-453, 461-473, 485-498, 501-525, 527-574, and/or 117-132 of SEQ ID NO: 1251; 4-11, 37-52, 56-64, 71-82, 89-96, 108-116, 122-137, 144-162, 165-182, 184-194, 228-237, 252-267, 289-296, 473-487, 489-503, 511-516, 527-545, 553-570, 584-593, 604-611, 629-638, 640-649, 684-696, and/or 536-549 of SEQ ID NO: 1252; 21-37, 81-97, 119-127, 130-143, 158-163, 175-180, 219-230, 245-256, 265-273, 293-299, 319-327, 425-434, 472-485, 493-498, 508-513, 552-557, 562-570, 574-580, 588-594, 597-604, 631-636, 640-646, 667-680, 699-718, 725-747, and/or 168-235 of SEQ ID NO: 1253; 4-11, 19-29, 51-56, 65-73, 85-98, 109-121, 125-135, 145-161, 171-177, 204-219, 223-228, 252-258, 262-268, 286-313, 315-325, 327-332, 345-352, 395-410, 429-435, 443-451, 455-463, 465-475, 481-487, 516-522, 549-562, 585-591, 598-605, 607-614, 643-651, 673-682, 690-696, 700-705, 725-734, 738-744, 758-765, 769-779, 781-792, 830-844, 847-853, and/or 700-722 of SEQ ID NO: 1254; 10-40, 68-91, 95-104, 140-152, 158-170, 185-191, 196-201, 205-219, 240-246, 249-256, 262-268, 274-280, 293-313, 315-320, 326-332, 338-358, 402-453, 457-466, 476-516, and/or 31-94 of SEQ ID NO: 1255; 10-34, 41-47, 50-66, 73-92, 100-107, 121-127, 133-139, 146-155, 159-175, 184-191, 223-230, 238-247, 273-286, 300-310, 328-336, 352-358, 365-372, 376-409, 415-423, 446-452, 459-465, 471-484, 509-522, 532-540, 543-554, 586-598, 600-610, 617-632, 670-689, 695-706, 711-727, 741-746, 752-760, 772-835, 843-882, 890-933, 958-973, 991-1022, 1024-1048, 1053-1070, and/or 277-365 of SEQ ID NO: 1256; 4-31, 56-70, 77-96, 112-117, 124-137, 139-155, 160-169, 176-193, 228-234, 237-257, 271-288, 317-322, 337-371, 373-391, and/or 317-353 of SEQ ID NO: 1257; 4-14, 20-28, 30-52, 54-62, 76-84, 94-100, 125-132, 140-181, 185-191, 208-222, and/or 181-199 of SEQ ID NO: 1258; 9-46, 53-59, 74-80, 82-93, 97-103, 111-117, 130-142, 152-161, 188-197, 236-251, 264-271, 285-295, 307-340, 343-394, 397-412, 416-436, 440-472, 497-527, 540-548, 561-566, 573-583, 591-598, 607-616, 624-631, 639-649, 669-675, 683-689, 694-718, 728-736, 756-771, 782-803, 814-829, 831-871, 873-879, 882-897, 900-912, 920-928, 940-953, 963-998, and/or 243-318 of SEQ ID NO: 1259; 5-16, 24-30, 34-39, 45-51, 59-72, 80-86, 100-108, 116-123, 133-140, 148-162, 176-181, 184-211, 219-228, 233-242, 257-278, 300-310, 320-338, 340-345, 352-360, and/or 226-320 of SEQ ID NO: 1260; 5-12, 51-58, 61-74, 95-112, 124-137, 153-158, 163-189, 192-204, 209-236, 240-250, 255-273, 304-317, 320-326, 334-348, 350-356, 360-378, 384-416, 439-457, 465-470, 488-493, 496-505, 531-541, 548-557, 579-587, 593-601, 616-647, 649-659, 679-685, 693-702, 705-713, 715-734, 737-743, 751-758, 763-779, 781-788, 791-801, 856-862, 882-896, 903-914, and/or 38-55 of SEQ ID NO: 1261; 8-34, 51-57, 68-76, 79-95, 110-116, 127-140, 142-154, 162-172, 174-202, 214-220, 228-239, 292-306, 314-320, 322-329, 337-344, 356-371, 374-380, 382-393, 416-421, 424-433, 444-453, 461-468, 470-478, 485-490, 497-503, 511-519, 537-543, 555-564, 566-579, 588-594, 603-609, 615-627, 634-640, 647-659, 663-689, 699-710, 728-735, 739-748, 750-756, 764-769, 776-788, 418-441, and/or 511-591 of SEQ ID NO: 1262; 12-19, 21-30, 32-39, 43-49, 55-68, 76-87, 97-104, 114-123, 130-141, 153-168, 179-205, 207-216, 218-226, and/or 223-239 of SEQ ID NO: 1263; 8-34, 72-121, 128-141, 153-172, 174-216, 221-256, 261-294, 307-315, 332-349, 354-370, 372-435, 452-457, 461-477, 487-492, 503-509, 511-520, 537-549, 559-565, 568-582, 584-591, 593-602, 607-617, 625-638, 655-674, 681-687, 696-703, and/or 254-268 of SEQ ID NO: 1264; 4-13, 23-40, 79-98, 106-124, 126-149, 154-161, 163-176, 178-187, 199-226, 232-254, 256-276, 297-304, 308-326, 329-338, 364-373, 384-399, 404-432, 439-499, 502-518, 523-544, 557-568, 571-582, 584-590, 603-617, 621-627, 633-639, 641-651, 653-663, 675-684, 686-699, 705-729, 736-781, and/or 522-597 of SEQ ID NO: 1265; 5-45, 49-62, 85-99, 114-122, 136-148, 151-171, 198-211, 253-260, 278-287, 297-303, 309-314, 318-324, 326-336, 348-363, and/or 1-115 of SEQ ID NO: 1266; 5-13, 22-33, 88-95, 132-144, 160-166, 189-202, 210-223, 253-258, 269-282, 286-294, 83-98, and/or 244-269 of SEQ ID NO: 1267; 18-25, 29-38, 72-95, 97-107, 110-139, 144-152, 155-161, 174-182, 198-203, and/or 44-56 of SEQ ID NO: 1268; 5-14, 17-23, 29-50, 52-64, 72-98, 109-115, 120-135, 137-145, 152-158, 167-175, 178-185, 210-234, 241-255, 258-271, 276-281, 290-303, 307-312, and/or 10-34 of SEQ ID NO: 1269; 5-32, 50-56, 62-70, 78-84, 97-121, 132-171, 177-182, 188-195, 204-214, 241-250, 267-274, 276-281, 292-309, 311-320, 333-344, 349-361, 375-395, 398-405, and/or 383-400 of SEQ ID NO: 1270; 4-10, 16-26, 59-80, 82-93, 97-115, 117-131, 148-160, 169-182, 184-210, 217-234, 241-254, 256-263, 265-276, 306-312, 344-350, 384-395, 400-409, 416-423, 428-440, 449-465, 496-504, 517-555, 335-360, and/or 427-541 of SEQ ID NO: 1271; 4-20, 48-91, 96-115, 134-142, 171-187, 197-217, 222-242, 246-255, 264-270, 277-289, 305-320, 338-352, 354-373, and/or 21-130 of SEQ ID NO: 1272;6-23, 25-53, 61-76, 83-92, 107-121, 147-166, 186-201, 207-215, 243-251, 264-274, 282-326, 333-348, 357-366, 371-380, 401-423, 432-465, 471-477, 481-490, 500-506, 512-525, 540-560, 583-603, 605-612, 615-626, 647-656, 661-681, 687-693, 713-722, and/or 587-614 of SEQ ID NO: 1273; 4-9, 15-21, 28-35, 39-54, 59-73, 76-86, 92-108, 120-134, 136-142, 145-161, 209-217, 220-228, 236-249, 258-267, 275-282, 293-304, 306-327, 330-340, 346-352, 354-360, 368-383, 386-392, 401-413, and/or 97-175 of SEQ ID NO: 1274; 4-11, 20-30, 47-66, 72-97, 108-117, 119-129, 142-163, 211-219, 224-237, 243-249, 251-263, 270-288, 295-305, 311-316, 326-333, 341-346, 367-375, and/or 301-378 of SEQ ID NO: 1275; 22-30, 38-45, 62-68, 78-90, 96-103, 107-114, 118-127, 134-148, 150-173, 179-193, 195-200, 205-219, 221-234, 239-248, 250-280, 282-296, 308-325, 334-351, 363-389, 425-432, 438-443, 468-481, 488-495, 499-517, 570-593, 602-610, 613-621, 629-637, and/or 536-559 of SEQ ID NO: 1276; 12-23, 26-35, 51-63, 72-78, 86-91, 135-140, 183-190, 201-209, 211-219, 241-247, 260-266, 272-281, 295-301, 329-335, 339-345, 355-366, 387-402, 428-445, 453-459, 503-517, 519-527, 531-538, 546-553, 560-569, 3-131, and/or 232-331 of SEQ ID NO: 1277; 8-13, 25-35, 51-56, 72-78, 86-91, 135-140, 183-190, 201-209, 211-219, 241-247, 260-265, 272-281, 295-301, 329-335, 339-345, 355-366, 387-402, 428-445, 453-459, 503-512, 519-529, 531-538, 546-553, 560-569, 1-145, 160-183, 212-334, and/or 376-400 of SEQ ID NO: 1278; 5-17, 62-71, 73-116, 118-131, 137-144, 151-158, 160-167, 169-175, 181-190, 193-210, 212-222, 231-262, 273-280, 300-329, 341-358, 363-368, 394-400, 403-409, 416-427, 450-457, 464-470, 478-484, 499-511, 513-529, 544-554, 558-565, 573-589, 597-604, and/or 335-348 of SEQ ID NO: 1279; 4-18, 38-46, 52-60, 65-79, 93-115, 123-131, 144-154, 168-183, 191-196, 201-223, 225-236, 250-263, 273-278, 289-317, 328-338, 357-373, 384-399, and/or 159-248 of SEQ ID NO: 1280; 11-39, 43-52, 57-69, 72-98, 112-142, 147-154, 159-165, 167-178, 198-210, 213-227, 244-250, 257-266, 268-286, 295-301, 305-311, 318-338, 340-346, and/or 262-323 of SEQ ID NO: 1281; 4-9, 17-23, 40-49, 57-70, 72-87, 92-121, 124-133, 135-146, 158-164, 173-195, 204-213, 215-221, 230-246, 250-257, 260-273, 280-298, 304-309, 311-328, 336-343, 362-371, 373-406, 409-418, 433-446, 450-456, 490-496, 503-513, 526-542, 548-563, 569-592, and/or 185-212 of SEQ ID NO: 1282; 4-17, 23-34, 36-44, 53-62, 72-85, 87-92, 110-115, 118-123, 129-150, 155-168, 172-180, 191-197, 205-211, 213-223, 225-233, and/or 176-201 of SEQ ID NO: 1283; 8-26, 33-44, 52-72, 78-96, 145-151, 154-171, 204-212, 223-230, 236-251, 261-272, 280-341, 365-374, 385-394, 417-423, 434-447, 456-486, 494-500, 509-519, 530-546, 556-566, 568-579, 581-603, and/or 300-322 of SEQ ID NO: 1284; 5-11, 14-21, 26-45, 52-67, 71-79, 82-97, 104-144, 151-159, 183-189, 194-205, 211-223, 241-252, 265-273, 275-280, and/or 93-116 of SEQ ID NO: 1285; 13-20, 55-72, 102-109, and/or 10-107 of SEQ ID NO: 1286; 5-14, 16-22, 38-45, 51-59, 61-78, 94-102, 133-142, 153-160, 187-195, 208-221, 240-254, 256-262, 270-275, 281-287, 294-299, 338-354, 356-364, 369-378, 446-452, 506-515, 557-564, 576-599, and/or 132-242 of SEQ ID NO: 1287; 4-29, 41-57, 66-75, 86-93, 96-102, 109-116, 124-131, 164-171, 188-194, 199-208, 214-231, 289-295, 305-310, 314-319, 336-351, 362-368, 389-399, 403-412, 422-433, 435-441, 444-461, 463-469, and/or 250-326 of SEQ ID NO: 1288; 4-16, 46-56, 59-73, 85-94, 97-105, 127-144, 160-166, 188-194, 233-238, 245-250, 270-275, 286-291, and/or 59-76 of SEQ ID NO: 1289; 5-33, 58-64, 78-116, 119-127, 139-160, 171-190, and/or 33-56 of SEQ ID NO: 1290; 6-11, 19-39, 50-56, 60-66, 83-133, 135-167, 195-216, 226-260, 271-319, 327-339, 342-355, 362-368, 373-394, and/or 170-191 of SEQ ID NO: 1291; 4-37, 45-51, 106-119, 132-138, 150-156, 160-171, 176-182, 193-203, 207-215, 222-229, 237-244, 253-261, 296-372, 403-409, 416-422, 440-449, 451-460, 471-485, 504-510, 538-551, 560-569, 573-582, 585-597, 606-617, 632-646, 658-666, 668-676, 685-694, and/or 552-658 of SEQ ID NO: 1292; 16-32, 53-79, 81-96, 102-111, 124-130, 145-152, 159-168, 176-189, 206-231, 236-244, 251-258, 286-292, 304-310, 315-326, 351-361, 383-390, 392-398, 405-411, 430-436, 456-464, 470-480, 482-496, 505-513, 531-540, 546-558, 583-589, 601-607, 621-634, 638-644, 667-673, 681-687, 693-702, 721-733, 737-744, 747-757, 760-767, 772-789, 796-807, 818-823, 846-852, 856-866, 868-880, 882-890, 913-919, 923-929, and/or 14-34 of SEQ ID NO: 1293; 19-42, 55-67, 75-95, 144-156, 168-175, 183-189, and/or 98-109 of SEQ ID NO: 1294; 7-17, 22-27, 34-61, 88-97, 110-117, 152-159, 175-191, 202-213, 220-232, 267-285, 296-315, 341-347, 376-392, 400-408, 421-430, 453-462, 464-470, 478-485, and/or 199-269 of SEQ ID NO: 1295; 27-44, 71-80, 114-123, 127-140, 149-167, 175-188, 191-202, 205-217, 222-227, 270-276, 297-302, 308-318, 324-332, 349-368, 370-376, 382-393, 432-441, 445-459, 472-481, 489-496, and/or 3-25 of SEQ ID NO: 1296; 16-49, 57-63, 84-92, 100-120, 124-130, 160-183, 189-200, 202-209, 236-244, 263-280, 283-334, 341-346, 377-389, 405-423, 452-458, 485-493, 505-513, 518-530, 547-559, 568-579, 595-601, 619-625, 638-649, and/or 474-504 of SEQ ID NO: 1297; 4-18, 21-30, 43-54, 76-83, 85-106, 116-122, 124-160, 180-185, 198-204, 206-222, 230-241, 258-263, 270-302, 325-332, 359-371, 374-386, 391-402, 411-417, 435-443, 458-485, and/or 376-395 of SEQ ID NO: 1298; 17-26, 28-34, 54-62, 83-93, 108-114, 119-125, 151-164, 175-187, 197-222, 224-232, 234-250, 265-270, 276-313, 315-342, 373-383, and/or 132-153 of SEQ ID NO: 1299; 25-42, 47-53, 55-80, 86-113, 116-126, 128-135, 151-159, 167-174, 179-188, 207-221, 226-248, 257-266, 269-279, 297-304, 312-325, and/or 144-155 of SEQ ID NO: 1300; 16-26, 61-71, 75-85, 95-113, 126-163, 175-184, 202-246, 286-291, 293-302, 320-349, and/or 57-76 of SEQ ID NO: 1301; 4-19, 29-36, 48-59, 92-103, 117-139, 154-164, 190-201, and/or 87-113 of SEQ ID NO: 1302; 43-51, 98-108, 123-130, 149-159, 168-179, 182-188, 201-207, 231-240, 334-340, 346-354, 369-378, 462-472, 493-503, 510-518, 520-538, 565-572, 584-590, 592-598, 616-628, 637-646, 659-675, 688-694, 699-705, 724-732, 740-755, 771-780, 792-801, 827-833, 846-856, 873-880, 882-906, 676-694, and/or 855-881 of SEQ ID NO: 1303; 15-45, and/or 29-60 of SEQ ID NO: 1304; 4-11, 15-34, 49-77, 92-121, 128-139, 169-176, 181-210, 216-222, 225-231, 233-246, 248-264, 271-276, 282-306, 319-326, 346-352, and/or 250-277 of SEQ ID NO: 1305; 6-20, 46-54, 64-79, 81-87, 131-138, 156-162, 170-178, 190-198, 226-232, 254-263, 266-274, 293-303, 313-323, 363-369, 419-424, 426-432, 436-455, 507-513, 515-521, 534-547, 553-559, 596-610, 617-636, 638-647, 659-665, 677-683, 691-696, 710-718, 724-730, 750-755, 770-780, 802-808, 824-842, 857-862, and/or 436-529 of SEQ ID NO: 1306; 11-25, and/or 21-35 of SEQ ID NO: 1307; 1-12 of SEQ ID NO: 1308;4-10, and/or 2-11 of SEQ ID NO: 1309; 12-20, and/or 5-14 of SEQ ID NO: 1310; 3-10 of SEQ ID NO: 1311; 4-22, and/or 10-21 of SEQ ID NO: 1312; 4-30, 35-50, and/or 17-32 of SEQ ID NO: 1313; 1-20 of SEQ ID NO: 1314; 16-21, and/or 7-22 of SEQ ID NO: 1315; 5-13, and/or 7-18 of SEQ ID NO: 1316; 10-17, and/or 16-29 of SEQ ID NO: 1317; 17-27, and/or 22-34 of SEQ ID NO: 1318; 12-18, and/or 9-21 of SEQ ID NO: 1319; 4-11, 13-94, 100-111, 115-134, and/or 89-106 of SEQ ID NO: 1320; 4-24, and/or 10-27 of SEQ ID NO: 1321; 4-14, and/or 12-23 of SEQ ID NO: 1322; 18-33, and/or 13-33 of SEQ ID NO: 1323; 16-23, 25-48, and/or 29-40 of SEQ ID NO: 1324; 4-12, 25-64, 68-76, and/or 4-61 of SEQ ID NO: 1325; 4-57, 59-66, 96-104, and/or 76-91 of SEQ ID NO: 1326; 23-43, 45-75, 97-107, 112-121, and/or 26-62 of SEQ ID NO: 1327; 10-24 of SEQ ID NO: 1328; 4-50, and/or 30-44 of SEQ ID NO: 1329; 4-20, 24-43, and/or 10-24 of SEQ ID NO: 1330; 4-49, 56-66, and/or 5-54 of SEQ ID NO: 1331; 33-44, and/or 15-38 of SEQ ID NO: 1332; 4-36, 39-50, and/or 32-47 of SEQ ID NO: 1333; 4-9, 16-30, 32-38, and/or 15-32 of SEQ ID NO: 1334; 4-9, and/or 27-42 of SEQ ID NO: 1335; 4-16, 32-43, 49-58, 64-72, and/or 14-27 of SEQ ID NO: 1336; 12-19, 24-29, 37-43, 47-53, 65-72, 83-95, 112-122, 136-147, 162-168, 174-181, 189-195, 201-208, 216-221, 234-243, 270-276, 278-288, 305-316, 318-342, 350-356, 368-400, 420-428, 434-443, 471-477, 481-488, 530-535, 540-547, 566-575, 591-601, 603-609, and/or 624-629 of SEQ ID NO: 763; 11-22, 28-34, 40-45, 65-86, 99-107, 115-125, 132-141, 143-150, 158-190, 203-211, 216-239, 246-257, 259-270, 272-279, 286-306, 313-332, 338-364, 371-380, 389-397, 410-418, 422-435, 467-510, 515-521, 532-538, and/or 547-563 of SEQ ID NO: 764; 7-20, 28-45, 51-66, 81-104, 108-115, 124-137, 149-155, 161-206, 209-214, 222-239, 250-262, 274-282, 309-343, 351-363, 365-386, 405-413, 435-440, 446-454, 458-466, 470-477, and/or 482-492 of SEQ ID NO: 765; 10-25, 29-35, 39-46, 54-71, 82-88, 102-111, 122-137, 139-145, 152-160, 162-172, 176-182, 193-201, 209-218, 226-232, 242-249, 258-268, 299-314, 318-344, 362-376, 393-399, 405-418, 426-463, 473-485, 487-492, 498-503, 518-544, and/or 561-567 of SEQ ID NO: 766; 16-23, 66-90, 98-110, 125-131, 144-150, 194-200, 213-219, 221-232, 237-256, 263-281, 293-298, 311-318, 326-337, 339-354, 373-389, 396-402, 404-421, 427-439, 441-448, 452-462, 467-479, 508-530, 534-541, 544-550, 562-569, 575-581, 583-592, 595-628, 636-656, 658-672, 674-680, 687-697, 715-721, 731-736, 739-749, 754-761, 771-788, 790-797, and/or 813-824 of SEQ ID NO: 767; 14-42, 51-57, 66-77, 84-96, 103-111, 129-148, 158-193, 198-208, 212-222, and/or 242-262 of SEQ ID NO: 768; 4-23, 29-62, 65-84, 98-104, 128-135, 144-161, 167-173, 175-204, 219-240, 250-264, 266-278, and/or 280-290 of SEQ ID NO: 769; 13-26, 33-45, 50-60, 75-81, 97-105, 123-131, 138-145, 158-166, and/or 168-177 of SEQ ID NO: 770; 6-20, 23-44, 50-61, 67-82, 84-91, 104-125, 133-144, 149-156, 159-166, 173-180, 182-196, 200-206, 224-239, 245-288, 320-339, 342-349, 359-368, 377-385, 411-419, 427-435, 453-474, 481-489, 491-497, 505-514, 516-522, 536-564, 579-586, 618-631, 644-650, 654-661, 663-677, 679-689, 700-705, 710-753, 795-801, 809-814, 821-827, 842-851, 867-905, 920-925, 931-949, 954-961, 1017-1022, 1034-1040, 1047-1057, 1062-1075, 1081-1086, 1107-1117, 1119-1126, 1145-1154, and/or 1162-1172 of SEQ ID NO: 771; 13-21, 47-57, 72-83, 97-102, 105-119, 125-133, 146-153, 170-177, 221-239, 245-273, 283-291, 299-305, 317-329, 335-343, 358-367, 374-380, 399-407, 430-438, 449-454, 473-479, 483-505, 517-527, 531-537, 555-560, 586-599, 601-616, 623-629, 639-647, 649-654, 658-667, 669-676, 690-709, and/or 714-729 of SEQ ID NO: 772; 14-28, 34-40, 45-54, 69-76, 78-83, 86-100, 116-123, 135-143, 146-161, 168-179, 187-200, 204-225, 236-250, 255-265, 271-292, and/or 298-314 of SEQ ID NO: 773; 4-28, 36-42, 78-85, 106-122, 130-136, 144-150, 161-175, 180-190, 194-200, 226-234, 256-265, 274-294, 309-316, 324-333, 336-344, 373-379, 382-389, 398-404, 407-416, 422-446, 451-462, and/or 530-541 of SEQ ID NO: 774; 4-15, 17-42, 71-77, 80-86, 90-116, 123-135, 144-150, 153-163, and/or 183-194 of SEQ ID NO: 775; 7-13, 22-42, 56-62, 84-90, 102-112, 121-133, 140-148, 158-167, 173-181, 192-199, 227-234, 284-293, 301-307, 336-343, 345-353, 366-372, 376-397, 400-436, 439-450, 467-478, 504-510, 519-530, 532-547, 551-558, 564-575, 592-598, 619-630, 636-642, 655-661, 663-669, 671-679, 697-718, 724-736, 738-752, 759-773, 776-788, 805-823, 827-833, 842-852, 859-864, 874-881, 883-889, 905-914, 932-939, 941-957, 963-969, 978-991, 1011-1025, 1052-1062, 1067-1073, 1080-1088, 1106-1112, 1121-1132, and/or 1139-1152 of SEQ ID NO: 776; 9-23, 32-38, 44-54, 64-71, 95-126, 146-154, 163-173, 190-196, 199-206, 249-256, 262-277, 291-302, 350-356, 380-398, 406-413, 420-430, 433-444, 471-478, and/or 492-507 of SEQ ID NO: 777; 5-26, 57-66, 68-74, 81-87, 105-116, 122-132, 144-160, 185-212, 217-223, 228-234, 241-252, 269-274, 291-313, 318-328, 335-342, 368-375, 397-404, 411-422, 431-439, 462-472, 474-485, 493-499, 509-519, 521-527, 530-558, 563-579, and/or 590-602 of SEQ ID NO: 778; 9-23, 32-38, 44-54, 64-71, 95-126, 146-154, 163-173, 190-196, 199-206, 249-256, 262-277, 291-302, 350-356, 380-398, 406-413, 420-430, 433-444, 471-478, and/or 492-507 of SEQ ID NO: 779; 18-31, 34-53, 57-67, 74-81, 90-106, 136-144, 147-153, 157-163, 170-182, 192-207, 233-241, 245-251, 256-267, 274-281, 284-306, 318-330, 333-340, 345-351, 356-379, 388-404, 428-439, 455-466, 468-480, 488-505, 515-526, 553-564, 571-577, 594-600, 607-614, 616-628, 634-642, 644-651, 655-666, 672-678, 686-703, 732-738, 745-751, 755-768, 772-778, 785-805, 807-814, 817-825, 831-853, 858-868, 890-905, 918-926, 934-942, 957-970, 972-981, 990-1001, 1057-1067, 1069-1077, 1089-1109, 1116-1130, 1133-1141, 1154-1165, 1190-1206, 1208-1215, 1217-1225, 1228-1254, 1256-1263, 1271-1279, 1283-1305, 1333-1339, 1357-1367, 1373-1379, 1388-1405, 1432-1442, 1444-1451, 1453-1461, 1463-1479, 1488-1504, 1516-1524, 1532-1540, 1555-1568, 1589-1600, 1607-1613, 1633-1638, 1655-1665, 1687-1704, 1721-1728, 1731-1737, 1744-1763, 1793-1804, 1816-1823, 1833-1850, 1855-1865, 1868-1875, 1886-1902, 1916-1925, 1931-1937, 1954-1967, 1971-1977, 1987-2002, 2009-2015, 2030-2036, 2043-2049, 2053-2077, 2085-2098, 2114-2122, 2129-2136, 2154-2167, 2187-2203, 2232-2243, 2253-2274, 2286-2303, 2311-2323, 2344-2365, 2371-2378, 2388-2404, 2406-2413, 2415-2424, 2426-2450, 2454-2461, 2469-2475, and/or 2486-2505 of SEQ ID NO: 780; 4-22, 24-30, 33-39, 60-72, 83-89, 128-137, 153-161, 165-174, 186-194, 212-218, 251-260, 275-284, 288-297, 309-318, 335-341, 374-383, 399-407, 411-420, 432-440, 467-482, 518-526, 572-579, 595-609, 649-663, 680-686, 691-702, and/or 708-714 of SEQ ID NO: 781; 11-25, 39-57, 69-94, 100-107, 118-155, 158-171, 189-201, 226-233, 236-245, 249-262, 287-296, 298-312, 315-329, 333-342, 351-359, 364-374, 382-388, 399-407, 411-417, 419-449, 454-471, 486-492, 494-504, 515-541, 547-552, 582-600, 611-623, 625-641, 651-657, 678-692, 699-709, 713-720, 746-752, 772-781, 791-804, 829-844, 880-893, 900-910, 915-923, 936-942, and/or 953-970 of SEQ ID NO: 782; 5-11, 17-24, 26-32, 36-43, 50-61, 67-73, 91-102, 111-126, 133-148, 154-161, 167-173, 179-195, 208-223, 230-240, 242-253, 270-286, 292-306, 308-347, 352-371, 373-380, 386-395, 404-410, 418-433, 436-444, 447-460, 463-477, 486-492, 522-533, and/or 548-553 of SEQ ID NO: 783; 4-12, 15-27, 35-55, 68-95, 100-109, 117-122, 129-135, and/or 157-162 of SEQ ID NO: 784; 25-46, 59-65, 68-75, 83-90, 93-100, 107-115, 124-135, 151-177, 183-189, 194-206, 209-215, 219-224, 251-263, 267-276, 305-311, 318-327, 332-338, 350-356, 380-396, 406-412, 414-423, 431-437, 453-461, 463-481, 483-491, 505-510, 513-523, 528-545, and/or 568-575 of SEQ ID NO: 785; 10-35, 42-59, 65-70, 76-85, 92-104, 149-155, 184-191, 234-243, 248-259, 268-277, 279-287, 391-398, 410-430, 445-454, 488-494, 498-504, 518-523, 530-538, 574-590, 615-623, 627-633, 652-660, 662-670, 674-683, 703-714, 720-728, 731-737, and/or 751-757 of SEQ ID NO: 786; 5-12, 39-51, 57-64, 67-84, 86-108, 124-130, 139-159, 167-179, 181-202, and/or 226-235 of SEQ ID NO: 787; 12-20, 29-40, 57-77, 79-88, 97-103, 111-117, 119-137, 174-200, 202-218, 221-229, 231-238, 240-246, 254-264, 266-280, 296-308, and/or 321-331 of SEQ ID NO: 788; 4-18, 20-48, 54-68, 80-105, 110-117, 120-130, 132-167, 179-214, 227-246, 259-295, 306-323, 332-339, 345-351, 357-363, 366-374, and/or 379-392 of SEQ ID NO: 789; 8-21, 23-31, 53-66, 69-94, 99-113, 119-184, 190-214, 233-244, 268-274, 279-284, 289-298, 300-311, 315-337, 344-350, 364-383, and/or 385-397 of SEQ ID NO: 790; 26-38, 43-63, 67-76, 79-98, 105-112, 115-121, 132-144, 148-153, 179-184, 194-203, 239-245, 261-278, and/or 282-315 of SEQ ID NO: 791; 13-22, 24-30, 49-61, 65-72, 90-97, 99-105, 115-131, 152-160, 165-171, 176-188, 202-221, 231-250, 255-274, 280-286, 288-296, 331-337, 339-347, 350-358, 374-385, 391-408, 418-427, 438-453, 468-476, 482-490, 497-506, 526-532, 534-583, 696-702, 713-719, 730-748, 750-758, 762-778, 802-808, 825-857, 864-950, 963-1004, 1015-1023, and/or 1046-1058 of SEQ ID NO: 792; 4-10, 22-28, 35-44, 58-66, 74-84, 86-98, 116-131, 138-143, 181-186, 224-230, 241-253, 282-292, 305-313, 325-331, 333-341, 348-360, 384-391, 395-408, 415-429, 431-445, 525-531, 558-564, 567-584, 601-612, 624-637, 645-652, 682-687, and/or 700-706 of SEQ ID NO: 793; 4-13, 19-27, 33-40, 57-82, 107-115, 117-125, 162-173, 197-206, 215-226, 256-266, 291-298, 303-310, 318-323, 331-336, 345-360, 379-396, 405-410, 426-434, 454-462, 468-476, 482-497, 512-518, 529-541, 556-564, 570-581, 589-594, 601-610, 629-637, 639-648, 663-670, 704-711, 725-738, 746-768, 770-780, 787-804, 817-823, 830-837, 876-882, 930-936, and/or 939-968 of SEQ ID NO: 794; 5-13, 20-65, 67-74, 107-115, 128-169, 171-195, 238-244, 256-287, and/or 292-298 of SEQ ID NO: 795; 7-13, 16-77, 89-96, 104-114, 117-125, 148-160, 167-191, 193-202, and/or 227-236 of SEQ ID NO: 796; 21-36, 41-47, 54-89, 122-129, 138-165, 173-190, 196-216, and/or 221-229 of SEQ ID NO: 797; 8-45, 135-140, 172-182, 189-196, 206-216, 218-235, 260-269, 272-278, 307-313, 333-344, 352-359, 371-395, 403-414, 416-422, 426-438, 451-470, 478-484, 493-502, 504-511, and/or 514-533 of SEQ ID NO: 798; 6-25, 49-59, 65-96, 107-115, 117-124, 135-151, 176-185, and/or 203-209 of SEQ ID NO: 799; 5-15, 46-56, 58-81, 83-111, 118-138, 152-160, and/or 165-175 of SEQ ID NO: 800; 7-16, 18-24, 36-43, 54-60, 65-73, 88-94, 107-113, 122-128, 134-141, 162-171, 182-216, 218-235, 249-263, 266-278, 290-301, and/or 308-338 of SEQ ID NO: 801; 4-14, 19-24, 27-36, 38-51, 63-73, 90-96, 102-121, 138-150, 157-174, 176-202, 212-225, 229-245, 250-258, 261-268, 279-291, 293-310, 319-338, 358-368, 371-389, 393-398, 404-413, 416-433, 435-442, and/or 458-471 of SEQ ID NO: 802; 17-40, 47-82, 85-93, 101-113, 153-172, 180-186, 190-208, 215-224, 252-261, 269-279, 283-289, 294-306, 311-328, 397-408, 416-423, 425-437, 492-499, 513-534, 542-548, and/or 550-555 of SEQ ID NO: 803; 8-17, 29-43, 45-52, 58-69, 87-100, 102-112, 148-163, 172-187, 190-208, 210-227, 232-239, 245-253, 258-263, 286-299, 313-334, 346-362, 373-388, 391-410, 417-423, 425-430, 434-446, 457-472, 483-489, 496-502, 518-524, 537-546, 555-560, 602-610, 637-646, 676-689, 698-704, 706-742, 750-778, 780-791, 806-842, 864-879, 881-888, 890-899, 901-908, 910-921, 941-947, 953-959, 967-980, 990-995, 1000-1061, 1073-1079, 1081-1092, 1096-1118, 1121-1168, 1174-1185, 1195-1209, 1219-1232, 1237-1243, 1250-1274, 1276-1286, 1302-1319, 1324-1333, 1339-1344, 1349-1361, 1370-1376, 1418-1427, 1435-1449, 1453-1469, 1473-1478, 1482-1495, 1509-1517, and/or 1519-1526 of SEQ ID NO: 804; 4-14, 16-32, 42-47, 65-71, 82-109, 128-145, 158-171, 177-191, 197-228, and/or 230-236 of SEQ ID NO: 805; 16-22, 26-42, 52-72, 75-89, 97-102, 114-147, 154-160, 165-170, 172-200, 202-229, 231-244, 256-261, 267-278, 286-294, 312-319, 330-336, 340-346, 360-373, 375-383, 386-396, 420-441, 443-474, 484-491, 496-517, 535-574, 600-606, 608-624, 636-643, 646-658, 664-687, 692-703, 716-725, 733-750, and/or 755-764 of SEQ ID NO: 806; 4-14, 24-34, 47-69, 81-90, 98-112, 144-153, 161-169, 189-196, 202-208, 213-220, 243-249, 256-262, 265-271, 279-286, 299-307, 310-324, 326-345, 356-369, 397-416, 424-429, and/or 432-441 of SEQ ID NO: 807; 4-9, 14-23, 50-56, 59-68, 77-102, 111-120, 126-152, 161-167, 174-180, 189-202, 204-228, 237-245, 259-266, 278-285, and/or 300-309 of SEQ ID NO: 808; 23-35, 71-77, 94-100, 134-140, 157-163, 189-195, 211-219, 221-231, 238-244, 246-253, 263-277, 298-306, 315-321, 337-342, 350-355, 369-377, 389-400, 408-416, 422-427, 441-449, 465-477, 481-488, 527-532, and/or 534-551 of SEQ ID NO: 809; 14-20, 35-48, 53-63, 71-77, 95-101, 114-121, 123-133, 144-151, 153-160, 162-170, 190-197, and/or 201-211 of SEQ ID NO: 810; 9-17, 26-46, 65-72, and/or 90-101 of SEQ ID NO: 811; 21-39, 46-53, 68-96, 107-113, 118-124, 126-135, 158-185, 196-202, 204-213, 219-226, 246-253, 267-275, 277-285, 299-317, 319-338, 404-410, 421-428, and/or 435-463 of SEQ ID NO: 812; 17-24, 29-40, and/or 47-56 of SEQ ID NO: 813; 17-25, 32-77, 82-91, 100-128, 161-169, 194-207, 211-218, 227-232, 239-245, 255-260, 278-300, 311-325, 342-356, 382-390, 393-401, 416-460, 467-487, 491-503, 505-512, 516-532, 551-565, 568-575, 594-601, 610-632, 638-643, 647-670, 672-685, 699-710, and/or 712-726 of SEQ ID NO: 814; 5-14, 25-46, 49-55, 59-65, 77-85, 98-107, 125-169, 181-186, 223-240, 271-281, 290-300, 306-313, 315-322, 330-337, 348-359, 370-377, 384-392, 416-424, 428-445, 456-469, 479-486, 502-510, 518-523, 525-535, 555-575, 578-585, 605-612, 624-637, 661-685, 693-700, 708-721, 723-729, 744-754, and/or 770-775 of SEQ ID NO: 815; 4-33, 38-52, 92-106, 116-124, 133-138, 142-148, 153-159, 161-168, 245-257, 282-287, 314-321, 331-336, 355-361, 366-372, 374-390, 396-409, 447-455, 484-490, 498-504, 511-519, 531-538, 540-545, 574-581, 586-596, 625-631, 644-655, 668-674, 685-692, 718-723, 728-741, 771-778, 787-797, 801-807, 819-828, and/or 832-844 of SEQ ID NO: 816; 5-25, 31-39, 72-79, 93-102, 104-110, 122-132, 138-146, 157-189, 192-198, 205-214, 226-233, 240-248, 269-275, 282-298, 304-310, 313-327, and/or 342-348 of SEQ ID NO: 817; 12-39, 49-55, 59-69, 95-104, 106-111, 116-128, 161-184, 186-217, 229-237, 240-252, 254-269, 271-278, 311-326, 331-338, 348-356, 364-370, 375-408, 429-460, 471-482, 484-500, 508-516, 527-536, 539-548, 560-576, 583-605, 643-655, 662-676, 682-687, 691-697, 703-715, 726-734, 737-746, 757-768, 778-789, 791-814, 821-826, 834-854, 890-899, 914-925, 947-954, 959-967, 984-990, 993-1000, 1012-1021, 1039-1044, 1065-1070, 1081-1098, 1136-1142, 1149-1157, 1165-1170, 1175-1186, 1191-1201, 1225-1265, 1276-1285, 1292-1300, 1323-1333, 1351-1361, 1366-1372, 1383-1397, 1404-1412, 1417-1425, 1431-1448, 1468-1473, 1483-1494, 1496-1504, and/or 1506-1530 of SEQ ID NO: 818; 18-53, 64-93, 95-105, 124-135, 143-148, 155-161, 163-171, 184-198, 238-245, 258-271, 273-284, 287-292, 302-310, 312-320, 322-341, 349-365, 377-403, 407-414, 417-423, 444-453, 455-469, 471-495, 503-511, 536-557, 579-586, 588-609, 619-626, 632-638, 643-649, 656-663, 669-680, 682-688, 699-714, 729-739, 755-761, 768-776, 781-793, 801-815, 821-826, 833-842, and/or 863-869 of SEQ ID NO: 819; 8-15, 24-40, 51-65, 78-89, 102-111, 117-154, 164-177, 181-192, 198-209, 216-222, 230-237, 241-248, 254-268, 285-293, 298-321, 331-338, 366-373, 384-389, 392-415, 429-439, 441-451, 453-459, 471-486, 489-501, and/or 524-535 of SEQ ID NO: 820; 10-18, 26-38, 48-54, 60-69, 77-83, 88-95, 119-126, 133-169, 172-185, 193-206, 214-225, 236-250, 269-275, 278-301, 320-329, 336-341, 347-353, 356-369, and/or 389-396 of SEQ ID NO: 821; 27-32, 37-50, 68-82, 84-108, 134-145, 149-154, 162-170, 172-182, 194-200, 205-224, 232-270, 293-299, and/or 312-328 of SEQ ID NO: 822; 8-14, 19-45, 65-75, 82-87, 95-105, 135-149, 154-160, 175-184, 205-226, 229-244, 249-256, 284-296, 298-304, and/or 321-348 of SEQ ID NO: 823; 4-10, 15-24, 26-53, 55-71, 78-83, 90-112, 128-148, 156-163, 165-179, 203-213, 228-239, 250-259, 277-285, 292-314, 322-330, 335-340, 345-360, 363-370, 381-396, 404-409, and/or 416-427 of SEQ ID NO: 824; 20-32, 37-46, 53-65, 75-83, 86-95, 99-105, 121-133, 139-151, 183-190, 199-205, and/or 216-227 of SEQ ID NO: 825; 9-25, 29-48, 50-100, 102-126, 131-149, 167-173, 210-217, 224-256, 259-265, 275-292, 295-301, 308-313, 319-335, 337-346, 352-359, 362-382, 393-423, 436-449, 468-476, 481-487, 492-500, 526-534, 537-548, 560-567, 569-579, 590-598, 604-613, 629-636, and/or 644-656 of SEQ ID NO: 826; 4-18, 25-31, 33-39, 42-53, 64-92, 97-111, 123-129, 134-146, 165-171, 173-190, 192-213, 226-239, 251-273, 283-298, 316-324, 339-345, 350-356, 361-376, 400-408, 418-440, 444-451, 476-481, 503-516, 524-542, 555-563, 581-594, 607-629, 634-641, 647-670, 711-719, 728-738, 755-765, 772-780, 800-815, 822-833, 842-852, 860-865, 874-880, 891-913, 926-938, 941-946, 961-978, 984-990, 1014-1024, 1038-1044, 1052-1092, 1099-1111, 1120-1140, 1153-1168, 1170-1190, 1193-1210, 1221-1233, 1253-1264, 1268-1274, 1283-1289, 1295-1300, 1303-1327, 1338-1351, 1362-1368, 1391-1396, 1410-1416, 1429-1441, 1471-1477, 1483-1513, 1526-1555, 1585-1591, 1596-1630, and/or 1632-1639 of SEQ ID NO: 827; 10-25, 34-54, 57-67, 77-96, 111-121, 127-139, 151-157, 161-179, 183-198, 201-219, 233-239, 247-252, 268-276, 283-294, 299-309, and/or 319-324 of SEQ ID NO: 828; 7-15, 20-32, 85-97, 102-109, 117-133, 137-161, 176-184, 186-203, 213-225, 227-251, 258-274, 280-290, 319-325, 335-364, 377-387, 403-410, 412-421, 436-454, 458-475, 478-504, 512-521, 541-567, 601-609, 614-622, 635-651, 657-669, 687-694, 702-708, 717-733, 735-741, 766-786, 788-800, 813-818, and/or 823-834 of SEQ ID NO: 829; 4-38, 49-69, 75-85, 110-115, 127-134, 167-173, 203-211, 240-245, 258-264, 293-299, 301-316, 348-354, 356-362, 386-391, 405-410, 456-462, 474-483, 494-499, 511-516, 523-528, 533-538, 549-557, 579-585, 587-593, 607-612, 618-625, 627-634, 654-660, 664-670, 682-688, 697-702, 729-736, 749-756, 783-793, 804-812, 817-829, 862-868, 915-920, 944-950, 954-960, 1000-1031, 1044-1056, 1069-1077, 1079-1084, 1097-1118, 1139-1146, 1152-1158, 1165-1176, 1181-1186, 1201-1213, 1257-1263, 1268-1276, 1278-1285, 1354-1360, 1369-1378, 1386-1396, 1439-1446, 1491-1501, 1526-1548, and/or 1556-1564 of SEQ ID NO: 830; 4-13, 20-34, 47-53, 58-65, 76-82, 89-106, 139-160, 165-182, and/or 191-205 of SEQ ID NO: 831; 20-40, 42-49, 58-67, 71-80, 95-100, 116-122, 131-142, 145-152, 158-173, 179-186, 196-202, 229-241, 258-270, 289-300, 302-320, 345-351, 370-382, 391-404, 455-464, 504-514, and/or 516-527 of SEQ ID NO: 832; 5-14, 26-35, 38-45, 54-60, 63-79, 121-127, 137-145, 152-162, 167-173, 175-183, 191-202, 218-228, 238-263, 278-295, 303-316, 320-335, 337-345, 359-365, and/or 382-400 of SEQ ID NO: 833; 4-17, 31-38, 46-61, 68-73, 76-97, 128-139, 150-156, 166-172, 174-182, 184-212, 219-225, 238-245, and/or 249-262 of SEQ ID NO: 834; 4-24, 31-42, 45-54, 59-71, 83-92, 108-115, 123-130, 149-156, 202-208, and/or 224-233 of SEQ ID NO: 835; 4-16, 25-41, 44-52, 60-66, 73-82, 92-101, 108-114, 133-138, 145-155, 177-185, and/or 194-202 of SEQ ID NO: 836; 4-9, 21-39, 72-78, 82-88, 99-131, 136-143, 151-162, 164-187, 189-204, 208-216, 223-229, 232-240, 246-256, 269-283, 288-299, 311-321, and/or 328-335 of SEQ ID NO: 837; 26-33, 39-45, 50-62, 79-85, 87-101, 116-131, 142-152, 154-186, 193-199, 201-216, 221-243, 266-272, 281-297, 324-330, 335-342, 345-355, 375-383, and/or 407-413 of SEQ ID NO: 838; 4-22, 27-36, 60-69, 90-98, 107-113, 117-123, 127-134, 137-151, 154-161, 169-178, 185-192, 202-208, 214-223, 230-239, 245-255, 266-275, 307-317, 323-337, 339-353, 361-379, 385-391, 393-401, 415-422, 424-429, 434-442, 444-449, and/or 470-480 of SEQ ID NO: 839; 4-22, 29-34, 37-44, 53-80, 98-110, 127-142, 144-156, and/or 158-165 of SEQ ID NO: 840; 4-12, 14-20, 27-34, 39-47, 51-67, 69-81, 89-97, 105-119, 121-133, 140-149, and/or 151-161 of SEQ ID NO: 841; 17-24, 34-40, 78-85, 227-233, 294-315, 327-335, 345-351, 354-359, 363-368, 388-403, 405-411, 413-419, 425-434, 462-472, 480-500, 528-536, 542-560, 566-573, 579-589, 593-606, 614-646, 651-658, 663-669, 686-726, 734-747, 754-778, 787-806, 809-825, 827-839, and/or 876-887 of SEQ ID NO: 842; 9-29, 35-40, 49-63, 69-76, 110-134, 141-147, and/or 160-169 of SEQ ID NO: 843; 6-31, 33-47, 53-59, 62-78, 93-98, 105-114, 121-130, 136-169, 172-195, 197-208, 223-228, 236-267, 277-283, 295-307, 309-325, 330-339, 345-352, 358-370, 379-391, 419-450, 461-508, 510-519, 521-539, 547-575, 578-587, 589-603, 612-633, 644-657, 666-678, 694-706, 711-717, 728-742, 759-769, 777-784, 800-806, 820-838, 841-848, 851-856, 870-876, 887-895, 908-914, 923-940, 942-953, and/or 969-988 of SEQ ID NO: 844; 12-39, 41-50, 68-74, 87-97, 113-136, 141-156, 167-180, 190-196, 204-223, 229-235, and/or 247-278 of SEQ ID NO: 845; 10-16, 25-53, and/or 64-74 of SEQ ID NO: 846; 5-43, 46-81, 88-96, 137-142, 163-191, 195-203, 210-235, 241-254, 256-276, 280-288, 292-305, 307-313, 317-333, 335-343, 347-353, 357-363, 372-381, 384-389, and/or 399-409 of SEQ ID NO: 847; 8-14, 22-32, 35-46, 57-75, 83-91, 100-106, 108-114, 125-131, 133-142, 157-175, 186-211, 217-235, 246-361, 367-372, 382-394, 396-405, 414-438, 459-471, 504-510, 513-535, and/or 538-560 of SEQ ID NO: 848; 8-20, 25-30, 46-62, 67-73, 98-103, 105-114, 119-141, 144-153, 168-178, 181-193, 198-204, 208-227, 236-242, 249-258, 281-288, 291-306, 327-336, 340-361, 368-380, 389-409, 417-426, 428-435, 442-453, 468-486, 488-496, 498-509, 511-523, 540-553, 566-579, 587-603, 629-636, and/or 677-682 of SEQ ID NO: 849; 9-25, 41-61, 68-75, 81-102, 106-141, 158-165, and/or 172-191 of SEQ ID NO: 850; 7-38, 43-58, 67-79, 92-99, 103-111, 118-128, 130-139, 152-165, 170-186, 192-223, and/or 225-251 of SEQ ID NO: 851; 4-16, 21-36, 38-47, 54-64, 92-103, 117-126, 135-155, 157-200, 202-223, 231-239, and/or 246-262 of SEQ ID NO: 852; 6-38, 44-57, 62-75, 82-96, 104-109, 115-122, 129-136, 147-160, 185-193, 200-206, 230-245, 248-269, 274-282, 289-298, 306-314, 321-335, 344-362, 372-377, 385-394, 422-431, 438-447, 479-505, 529-541, 547-558, 564-571, 573-579, 606-612, 621-632, 638-649, 656-664, 676-683, 695-704, 711-716, 728-734, 736-742, 776-781, 783-791, 809-816, 850-856, 866-872, 889-898, 906-912, 919-926, 944-953, 987-992, 1015-1022, 1024-1038, 1066-1072, 1097-1105, 1113-1119, 1121-1136, and/or 1147-1154 of SEQ ID NO: 853; 5-14, 27-42, 48-67, 71-83, 85-91, 105-112, 114-135, 139-147, 159-165, 169-185, 188-195, 199-208, 212-221, 231-253, 264-272, 275-282, 290-303, 309-319, 324-331, 340-358, 380-405, 419-425, 438-444, 450-463, 468-477, 497-514, 520-533, 549-556, 568-574, 617-626, 637-643, 661-668, 674-684, 705-713, 718-733, and/or 735-769 of SEQ ID NO: 854; 5-16, 29-53, 63-71, 74-93, 124-132, 140-192, 199-216, 227-258, 260-268, 272-282, 285-300, 323-331, 353-368, 389-396, 414-421, 429-441, 448-454, 459-467, 474-493, 501-508, 516-575, 593-612, 631-661, 665-693, 715-724, 736-751, 754-765, 771-777, 782-791, 809-820, and/or 823-853 of SEQ ID NO: 855; 51-70, 81-95, 103-110, 117-123, 130-136, 142-160, 174-180, 199-207, 268-274, 280-296, 347-358, 361-369, 390-396, 401-409, 424-430, 442-448, 455-467, 501-508, 523-533, and/or 553-560 of SEQ ID NO: 856; 4-10, 35-45, 56-92, 108-116, 127-133, 137-146, 156-186, 194-203, 225-234, 242-251, 272-283, 285-306, 310-315, 322-330, 358-371, 373-379, and/or 389-396 of SEQ ID NO: 857; 4-32, 38-46, 66-83, 88-95, 110-118, 123-141, 169-180, 200-208, 217-225, 237-245, 247-261, 263-272, 275-282, 291-302, 310-338, 345-353, 360-369, 371-378, 386-394, 398-413, 416-422, and/or 437-448 of SEQ ID NO: 858; 4-11, 18-26, 31-47, 59-68, 74-92, 98-144, 149-158, 173-180, 200-210, 216-223, and/or 239-250 of SEQ ID NO: 859; 4-10, 29-56, 93-99, 119-124, 133-140, 159-171, 187-195, 200-214, 221-233, 249-255, 263-271, 285-291, and/or 310-316 of SEQ ID NO: 860; 29-35, 68-85, 92-99, 107-122, 124-135, 138-144, 146-158, 173-205, 208-227, 232-259, 277-285, 317-324, and/or 326-333 of SEQ ID NO: 861; 9-18, 21-27, 42-49, 58-75, 85-93, 100-106, 108-122, 125-131, 140-150, 155-162, 165-186, 201-206, 209-214, 220-249, 261-267, 279-289, 292-299, 304-310, 328-338, 343-355, 370-378, 381-389, 393-400, 411-418, 424-436, 438-449, 474-496, 524-531, 556-572, 586-592, 606-617, 623-629, 648-657, 659-676, 696-702, 709-717, 735-755, 763-775, 788-809, and/or 835-843 of SEQ ID NO: 862; 20-32, 52-63, 75-88, 96-101, 116-154, 164-173, 187-199, 202-240, 253-279, 285-298, 305-318, and/or 324-339 of SEQ ID NO: 863; 4-22, 24-29, 36-43, 63-75, 90-96, 118-128, 137-145, 168-182, 200-210, 212-221, 242-250, 289-316, 318-323, 327-339, 381-387, 401-411, 424-434, 443-449, 453-465, 485-498, 500-508, 510-515, 521-528, 538-545, 554-560, 574-606, 619-627, 645-658, and/or 681-688 of SEQ ID NO: 864; 8-18, 45-50, 52-62, 76-82, 84-107, 109-116, 130-137, 141-150, 152-158, 164-170, 175-186, and/or 188-196 of SEQ ID NO: 865; 11-19, 24-34, 41-48, 55-63, 68-81, 85-91, 100-106, 111-120, 131-138, 144-161, 168-176, 192-203, 213-225, 227-234, 236-243, 255-262, 265-274, 282-290, 296-301, 309-316, 349-359, 368-377, 384-390, 398-412, 417-433, 439-448, 467-475, 481-486, 501-508, 510-517, 521-532, and/or 538-545 of SEQ ID NO: 866; 4-42, 48-59, 74-88, 92-119, 121-149, 163-180, 185-195, and/or 199-209 of SEQ ID NO: 867; 5-26, 60-76, 104-114, 119-128, 136-141, 156-167, 186-198, 218-237, 260-267, 275-290, 309-318, and/or 328-335 of SEQ ID NO: 868; 5-17, 23-48, 60-73, 75-82, 98-108, 110-128, 146-160, 168-180, 191-213, 229-237, 240-252, 269-277, and/or 305-313 of SEQ ID NO: 869; 8-28, 53-62, 70-78, 81-89, 97-115, 125-148, 155-168, 174-186, 196-202, 207-214, 220-238, 241-256, 258-267, 284-290, 297-306, 312-317, 322-327, 330-344, 352-358, 367-385, 387-395, 400-414, 422-430, 438-455, 458-466, 491-507, 539-544, 561-566, 571-577, 598-604, 617-623, and/or 640-647 of SEQ ID NO: 870; 7-14, 24-32, 56-72, 95-100, 108-114, 123-138, 143-153, 203-221, 224-230, 260-269, 290-297, 302-308, 321-355, 364-370, 398-427, 434-439, 446-473, 505-510, 512-518, 536-546, 573-587, 589-595, 629-636, 683-709, 728-734, 778-786, 795-802, 825-830, 911-934, 944-956, 960-970, 977-985, 987-993, 1009-1015, 1027-1035, 1047-1052, and/or 1058-1065 of SEQ ID NO: 871;4-16, 21-36, 38-47, 54-64, 92-103, 117-126, 135-155, 157-200, 202-223, 231-239, and/or 246-262 of SEQ ID NO: 872; 4-10, 35-45, 56-92, 108-116, 127-133, 137-146, 156-186, 194-203, 225-234, 242-251, 272-283, 285-306, 310-315, 322-330, 358-371, 373-379, and/or 389-396 of SEQ ID NO: 873; 4-40, 47-59, 62-75, 81-87, and/or 107-113 of SEQ ID NO: 874; 6-13, 21-31, 44-54, 64-70, and/or 77-84 of SEQ ID NO: 875; 17-24, 26-41, and/or 50-55 of SEQ ID NO: 876; 24-32 of SEQ ID NO: 877; 15-28, and/or 36-44 of SEQ ID NO: 878; 4-25, 28-34, 37-43, 45-52, 59-69, 85-91, and/or 104-114 of SEQ ID NO: 879; 13-39, 51-63, 80-101, 141-149, 165-176, and/or 191-210 of SEQ ID NO: 880; 6-19 of SEQ ID NO: 881; 4-19, 23-42, 57-66, 98-114, 121-127, 148-155, 164-181, 194-204, 217-222, 248-258, 266-285, 288-296, 309-329, 331-342, 344-353, and/or 361-378 of SEQ ID NO: 882; 4-14, and/or 26-32 of SEQ ID NO: 883; 10-18, 29-62, 72-91, 106-120, 147-154, 185-195, 203-210, 220-238, 262-267, 275-285, 315-365, 376-388, 390-399, and/or 414-423 of SEQ ID NO: 884; 8-17, 43-49, 63-70, 79-86, and/or 89-100 of SEQ ID NO: 885; 4-19, 21-31, and/or 54-68 of SEQ ID NO: 886; 4-10, and/or 26-31 of SEQ ID NO: 887; 4-12, 21-30, and/or 37-51 of SEQ ID NO: 888; 4-11, and/or 20-46 of SEQ ID NO: 889; 4-10, and/or 23-28 of SEQ ID NO: 890; 4-28, and/or 32-50 of SEQ ID NO: 891; 4-25 of SEQ ID NO: 892; 35-53 of SEQ ID NO: 893; 14-20, 48-56, 63-68, and/or 79-87 of SEQ ID NO: 894; 34-40, and/or 50-62 of SEQ ID NO: 895; 23-34, and/or 59-72 of SEQ ID NO: 896; 4-16, 28-39, 62-68, and/or 85-97 of SEQ ID NO: 897; 10-20, 23-31, 35-42, and/or 48-62 of SEQ ID NO: 898; 4-44, 46-52, 54-64, and/or 70-85 of SEQ ID NO: 899; 4-12, and/or 24-40 of SEQ ID NO: 900; 10-22, 24-46, 52-57, and/or 74-81 of SEQ ID NO: 901; 10-20, 27-40, and/or 48-57 of SEQ ID NO: 902; 24-37 of SEQ ID NO: 903; 20-29, 39-45, 53-65, and/or 67-85 of SEQ ID NO: 904; 6-22, 25-41, 43-66, 74-84, 87-94, and/or 107-113 of SEQ ID NO: 905; 4-12, 17-23, and/or 39-62 of SEQ ID NO: 906; 4-13, 21-45, and/or 51-70 of SEQ ID NO: 907; 15-25, and/or 33-52 of SEQ ID NO: 908; 8-19, 44-53, 60-70, 78-85, and/or 97-107 of SEQ ID NO: 909; 13-23, 31-40, 59-66, 84-90, and/or 96-110 of SEQ ID NO: 910; 4-25, 37-48, 56-71, 83-97, 112-132, 140-150, and/or 152-157 of SEQ ID NO: 911; 4-17, 19-26, 41-49, 63-87, 92-99, and/or 113-131 of SEQ ID NO: 912; 4-10, and/or 17-23 of SEQ ID NO: 913; 4-19, 26-35, 43-50, and/or 61-72 of SEQ ID NO: 914; 5-10, 31-38, 42-70, and/or 84-99 of SEQ ID NO: 915; 12-26, and/or 48-55 of SEQ ID NO: 916; 4-10, 13-63, 69-81, 83-105, and/or 143-150 of SEQ ID NO: 917; 4-12, 18-29, and/or 49-68 of SEQ ID NO: 918; 19-25, 27-33, 43-84, 86-92, 111-118, 125-136, 138-147, 158-165, and/or 181-192 of SEQ ID NO: 919; 20-29, 50-56, 63-85, 89-98, and/or 120-128 of SEQ ID NO: 920; 4-11, and/or 41-47 of SEQ ID NO: 921; 14-27, and/or 50-62 of SEQ ID NO: 922; 10-19, 35-41, and/or 43-51 of SEQ ID NO: 923; 17-40, 47-66, and/or 70-78 of SEQ ID NO: 924; 549-576, and/or 595-622 of SEQ ID NO: 1007; 592-616 of SEQ ID NO: 1031; 9-22 of SEQ ID NO: 1032; 896-923 of SEQ ID NO: 317; 178-204, 222-248, 244-269, and/or 265-296 of SEQ ID NO: 320; 180-209, and/or 205-233 of SEQ ID NO: 321; 242-270, and/or 266-294 of SEQ ID NO: 322; 718-740, 328; 197-229, and/or 225-256 of SEQ ID NO: 345; 42-61 of SEQ ID NO: 359; 34-53 of SEQ ID NO: 425; 1522-1552 of SEQ ID NO: 431; 415-440 of SEQ ID NO: 462; 32-51 of SEQ ID NO: 463; 342-369, and/or 457-484 of SEQ ID NO: 466; 554-582, 578-606, and/or 632-659 of SEQ ID NO: 467; 12-33, and/or 27-48 of SEQ ID NO: 469; 52-81, 77-105, and/or 101-129 of SEQ ID NO: 471; 76-102 of SEQ ID NO: 472; 48-77 of SEQ ID NO: 473; 6-35 of SEQ ID NO: 474; 189-218, and/or 214-244 of SEQ ID NO: 456; 122-149 of SEQ ID NO: 459; 106-136 of SEQ ID NO: 460; 204-224 of SEQ ID NO: 477; 273-297 of SEQ ID NO: 301; 70-88 of SEQ ID NO: 302; 134-147 of SEQ ID NO: 303; 290-317, and/or 367-388 of SEQ ID NO: 304; 240-265 of SEQ ID NO: 305; 796-824 of SEQ ID NO: 306; 83-110, and/or 106-133 of SEQ ID NO: 308; 120-148 of SEQ ID NO: 309; 71-106 of SEQ ID NO: 310; 278-305 of SEQ ID NO: 312; 115-137of SEQ ID NO: 313; 378-400 of SEQ ID NO: 315; 111-137 of SEQ ID NO: 316; 3-19 of SEQ ID NO: 318; 103-119 of SEQ ID NO: 323; 89-116, 112-139, and/or 135-162 of SEQ ID NO: 326; 259-268 of SEQ ID NO: 390; 2-19 of SEQ ID NO: 501; 83-114 of SEQ ID NO: 488; 15-33 of SEQ ID NO: 496; 52-84, and/or 70-93 of SEQ ID NO: 498; 28-54 of SEQ ID NO: 502; 34-66 of SEQ ID NO: 514; 4-32, and/or 28-56 of SEQ ID NO: 528; 21-47 of SEQ ID NO: 513; 32-57 of SEQ ID NO: 490; 96-115 of SEQ ID NO: 542; 161-176 of SEQ ID NO: 543; 58-89, 85-116, and/or 112-143 of SEQ ID NO: 557; 97-115 of SEQ ID NO: 559; 84-106 of SEQ ID NO: 565; 3-19 of SEQ ID NO: 572; 38-63 of SEQ ID NO: 575; 33-64, 60-91, and/or 87-119 of SEQ ID NO: 580; 93-123 of SEQ ID NO: 558; 16-34 of SEQ ID NO: 563; 61-92, and/or 88-120 of SEQ ID NO: 593; 48-76, 72-100, 96-124, and/or 120-147 of SEQ ID NO: 568; 111-146 of SEQ ID NO: 569; 26-50, 46-71, and/or 67-92 of SEQ ID NO: 570; 11-46 of SEQ ID NO: 573; 92-127 of SEQ ID NO: 574; 46-78, 74-106, and/or 102-133 of SEQ ID NO: 582; 94-124 of SEQ ID NO: 584; 2-29 of SEQ ID NO: 585; 10-43 of SEQ ID NO: 597; 24-51, 47-74, and/or 70-98 of SEQ ID NO: 600; 15-42, and/or 38-58 of SEQ ID NO: 551; 259-268 of SEQ ID NO: 390; 66-94 of SEQ ID NO: 598; 17-43 of SEQ ID NO: 594; 178-198 of SEQ ID NO: 375; 441-473, 469-501, and/or 497-531 of SEQ ID NO: 385; 105-127 of SEQ ID NO: 387; 8-35, 36-63, and/or 64-91 of SEQ ID NO: 388; 216-231 of SEQ ID NO: 391; 203-222 of SEQ ID NO: 395; 180-210, 206-236, and/or 232-261 of SEQ ID NO: 404; 6-28 of SEQ ID NO: 405; 224-238 of SEQ ID NO: 408; 274-291 of SEQ ID NO: 411; 134-148 of SEQ ID NO: 413; 133-153 of SEQ ID NO: 414; 258-284 of SEQ ID NO: 416; 8-33, 28-53, and/or 49-74 of SEQ ID NO: 419; 103-127, 123-147, 143-166, and/or 162-185 of SEQ ID NO: 420; 179-206 of SEQ ID NO: 421; 15-27 of SEQ ID NO: 434; 240-261 of SEQ ID NO: 435; 386-402 of SEQ ID NO: 437; 57-74 of SEQ ID NO: 438; 343-375, 371-403, and/or 399-432 of SEQ ID NO: 448; 369-390 of SEQ ID NO: 450; 276-292 of SEQ ID NO: 452; 196-227, 223-254, 250-280, 284-311, 307-334, 330-356, and/or 352-378 of SEQ ID NO: 453; 253-271 of SEQ ID NO: 454; 419-432 of SEQ ID NO: 455; 24-51 of SEQ ID NO: 370; 259-268 of SEQ ID NO: 390; 23-48 of SEQ ID NO: 371; 179-198 of SEQ ID NO: 372; 431-455 of SEQ ID NO: 373; 319-349, 345-374, and/or 370-399 of SEQ ID NO: 374; 455-479, and/or 475-498 of SEQ ID NO: 376; 653-675 of SEQ ID NO: 377; 75-108 of SEQ ID NO: 378; 1362-1388 of SEQ ID NO: 379; 8-36, 32-60, and/or 56-84 of SEQ ID NO: 380; 1-25 of SEQ ID NO: 381; 65-88 of SEQ ID NO: 382; 102-134 of SEQ ID NO: 383; 104-131 of SEQ ID NO: 384; 206-231 of SEQ ID NO: 386; 5-23 of SEQ ID NO: 389; 510-536, and/or 546-577 of SEQ ID NO: 390; 300-326, and/or 1320-1353 of SEQ ID NO: 392; 205-240 of SEQ ID NO: 393; 971-1003 of SEQ ID NO: 394; 455-483, and/or 1232-1263 of SEQ ID NO: 396; 12-40 of SEQ ID NO: 397; 3-32 of SEQ ID NO: 398; 68-103 of SEQ ID NO: 399; 188-222 of SEQ ID NO: 400; 25-55 of SEQ ID NO: 401; 98-130 of SEQ ID NO: 402; 211-236 of SEQ ID NO: 403; 362-392 of SEQ ID NO: 406; 365-389 of SEQ ID NO: 407; 60-81 of SEQ ID NO: 409; 69-104 of SEQ ID NO: 410; 124-152, and/or 148-177 of SEQ ID NO: 412;74-102 of SEQ ID NO: 415; 1019-1051, 1162-1190, 1186-1214, 1210-1238, and/or 1234-1261 of SEQ ID NO: 417; 692-724 of SEQ ID NO: 423; 259-268 of SEQ ID NO: 390; 15-41, 37-62, and/or 58-83 of SEQ ID NO: 418; 59-87, 83-110, and/or 111-140 of SEQ ID NO: 422; 174-206 of SEQ ID NO: 424; 96-119 of SEQ ID NO: 426; 128-152 of SEQ ID NO: 427; 104-130 of SEQ ID NO: 428; 542-568 of SEQ ID NO: 429; 107-129 of SEQ ID NO: 430; 112-137 of SEQ ID NO: 432; 52-80 of SEQ ID NO: 433; 19-45 of SEQ ID NO: 436; 1113-1144 of SEQ ID NO: 441; 94-119, 110-135, and/or 136-160 of SEQ ID NO: 439; 89-115 of SEQ ID NO: 442; 474-496, and/or 492-515 of SEQ ID NO: 443; 14-40 of SEQ ID NO: 444; 422-448 of SEQ ID NO: 445; 294-333 of SEQ ID NO: 447; 113-140 of SEQ ID NO: 449; 8-36, 32-61, and/or 57-86 of SEQ ID NO: 451; 1-28, 120-147, and/or 143-170 of SEQ ID NO: 440; -14-11 of SEQ ID NO: 446; 96-124 of SEQ ID NO: 1042; 550-575, 571-596, and/or 592-616 of SEQ ID NO: 1031; 144-170 of SEQ ID NO: 1046; 79-100 of SEQ ID NO: 1048; 73-96 of SEQ ID NO: 1050; 21-43 of SEQ ID NO: 1051; 18-42 of SEQ ID NO: 1052; 16-50 of SEQ ID NO: 1053; 7-25 of SEQ ID NO: 1055; 8-44 of SEQ ID NO: 1056; 22-51 of SEQ ID NO: 1057; 2-25 of SEQ ID NO: 1059; 9-44 of SEQ ID NO: 1060; 6-34, 30-58, 54-82, and/or 78-105 of SEQ ID NO: 1061; 9-32 of SEQ ID NO: 1062; 23-46, and/or 42-65 of SEQ ID NO: 1063; 1-25 of SEQ ID NO: 1064; 17-43 of SEQ ID NO: 1065; 11-38 of SEQ ID NO: 1067; 44-72, 68-95, and/or 91-118 of SEQ ID NO: 1009; 163-187 of SEQ ID NO: 1043; 586-612 of SEQ ID NO: 1011; 259-284, 283-313, 309-339, and/or 335-365 of SEQ ID NO: 1012; 311-333 of SEQ ID NO: 1016; 245-269, 474-504, 500-530, and/or 526-555 of SEQ ID NO: 1018; 270-302, 298-330, and/or 326-358 of SEQ ID NO: 1021; 58-86, 82-109, 105-133, 325-353, 349-378, and/or 374-403 of SEQ ID NO: 1023; 9-33 of SEQ ID NO: 1032; 96-124 of SEQ ID NO: 1042; 447-475, 471-498, and/or 494-521 of SEQ ID NO: 1022; 285-316, 312-343, 339-371, 367-399, 515-541, 537-564, and/or 560-586 of SEQ ID NO: 1027; 149-175, 171-197, and/or 193-217 of SEQ ID NO: 997; 43-73, 69-99, 95-124, 163-189, 185-210, 206-231, 227-252, 248-273, 269-294, 313-340, 336-362, 358-384, 456-481, 477-502, and/or 498-523 of SEQ ID NO: 998; 136-163, 159-186, 193-217, 253-279, 275-301, 447-474, 470-496, 492-518, 519-547, and/or 543-572 of SEQ ID NO: 999; 31-58, 54-80, 76-102, 166-196, 222-246, 242-266, 284-310, 306-332, and/or; 328-354 of SEQ ID NO: 1000; 96-124 of SEQ ID NO: 1042; 35-58, 54-77, and/or; 73-96 of SEQ ID NO: 1001; 200-229, 225-253, 249-277, 495-522, 518-544, and/or 540-566 of SEQ ID NO: 1002; 238-267, 268-300, and/or; 301-330 of SEQ ID NO: 1003; 6-36, 32-62, 58-87, 210-237, 233-260, 256-282, 496-522, 518-543, and/or; 539-564 of SEQ ID NO: 1004; 26-53, 468-501, 497-530, 49-76, 72-98, 235-268, 253-283, 279-309, 305-334, 425-455, and/or; 439-472 of SEQ ID NO: 1005; 7-40, and/or; 36-69 of SEQ ID NO: 1006; 11-38, 34-61, 57-84, 126-153, 149-176, 172-198, 265-290, 286-311, and/or 307-331 of SEQ ID NO: 1045; 115-137 of SEQ ID NO: 313; 11-35 of SEQ ID NO: 1307; 1-25 of SEQ ID NO: 1312;8-32 of SEQ ID NO: 1313; 5-29 of SEQ ID NO: 1317; 10-34 of SEQ ID NO: 1318; 82-106 of SEQ ID NO: 1320; 3-27 of SEQ ID NO: 1321; 9-33 of SEQ ID NO: 1322; 1-25 of SEQ ID NO: 1323; 16-40 of SEQ ID NO: 1324; 4-35, and/or 31-61 of SEQ ID NO: 1325; 67-91 of SEQ ID NO: 1326; 25-52 of SEQ ID NO: 1327; 20-44 of SEQ ID NO: 1329; 1-25 of SEQ ID NO: 1330; 5-32, and/or 28-54 of SEQ ID NO: 1331; 14-38 of SEQ ID NO: 1332; 23-47 of SEQ ID NO: 1333; 8-32 of SEQ ID NO: 1334; 18-42 of SEQ ID NO: 1335; 3-27 of SEQ ID NO: 1336; 181-203 of SEQ ID NO: 1203; 450-474 of SEQ ID NO: 1204; 200-224, 220-245, and/or 241-266 of SEQ ID NO: 1205; 50-79, and/or 75-104 of SEQ ID NO: 1206; 195-219 of SEQ ID NO: 1207; 26-53, and/or 49-77 of SEQ ID NO: 1208; 107-136, and/or 132-162 of SEQ ID NO: 1209; 197-221 of SEQ ID NO: 1210; 10-34 of SEQ ID NO: 1211;48-80, and/or 76-107 of SEQ ID NO: 1212; 159-191, 187-218, 434-464, and/or 460-489 of SEQ ID NO: 1213; 66-90 of SEQ ID NO: 1214; 51-75, and/or 666-690 of SEQ ID NO: 1215; 99-123 of SEQ ID NO: 1216; 251-275 of SEQ ID NO: 1217; 115-137 of SEQ ID NO: 313; 102-129, and/or 125-151 of SEQ ID NO: 1218; 30-54 of SEQ ID NO: 1219; 138-162 of SEQ ID NO: 1220; 79-103 of SEQ ID NO: 1221; 101-125 of SEQ ID NO: 1222; 126-154, 150-178, and/or 174-202 of SEQ ID NO: 1223; 322-346 of SEQ ID NO: 1224; 418-442 of SEQ ID NO: 1225; 126-156, 152-182, 178-208, 436-465, 461-489, and/or 485-513 of SEQ ID NO: 1226; 42-75, 71-104, 134-161, 157-184, 181-209, and/or 205-233 of SEQ ID NO: 1227; 131-155, and/or 262-286 of SEQ ID NO: 1228; 43-69, and/or 65-90 of SEQ ID NO: 1229; 468-492 of SEQ ID NO: 1230; 90-115, 111-135, 131-155, and/or 151-175 of SEQ ID NO: 1231; 495-522, and/or 518-545 of SEQ ID NO: 1232; 45-72, and/or 68-95 of SEQ ID NO: 1233; 94-121, and/or 117-144 of SEQ ID NO: 1234; 8-32, and/or 337-361 of SEQ ID NO: 1235; 943-973, and/or 969-1000 of SEQ ID NO: 1236; 191-215 of SEQ ID NO: 1237; 70-97, and/or 93-120 of SEQ ID NO: 1238; 800-824, 820-844, and/or 840-864 of SEQ ID NO: 1242; 115-137 of SEQ ID NO: 313; 341-363, 359-381, 376-402, 398-424, and/or 420-445 of SEQ ID NO: 1241; 186-216 of SEQ ID NO: 1243; 103-127 of SEQ ID NO: 1244; 19-50, and/or 46-77 of SEQ ID NO: 1245; 80-107, 103-129, and/or 125-151 of SEQ ID NO: 1246; 406-430 of SEQ ID NO: 1247; 795-827, and/or 823-855 of SEQ ID NO: 1248; 404-428 of SEQ ID NO: 1249; 66-90, 86-110, and/or 106-130 of SEQ ID NO: 1250; 107-131 of SEQ ID NO: 1251; 524-548 of SEQ ID NO: 1252; 193-217, 293-323, 319-349, 345-375, and/or 371-400 of SEQ ID NO: 1253; 481-505 of SEQ ID NO: 1254; 33-58, and/or 54-78 of SEQ ID NO: 1255; 281-308, 304-331, and/or 327-354 of SEQ ID NO: 1256; 321-352 of SEQ ID NO: 1257; 174-198 of SEQ ID NO: 1258; 248-273, 269-293, and/or 289-313 of SEQ ID NO: 1259; 249-281, and/or 277-310 of SEQ ID NO: 1260; 31-55 of SEQ ID NO: 1261; 415-439, 540-566, and/or 562-589 of SEQ ID NO: 1262; 213-237 of SEQ ID NO: 1263; 243-267 of SEQ ID NO: 1264; 545-583 of SEQ ID NO: 1265; 73-97, and/or 240-264 of SEQ ID NO: 1267; 115-137 of SEQ ID NO: 313; 48-73, 69-93, and/or 89-113 of SEQ ID NO: 1266; 9-33 of SEQ ID NO: 1269; 375-399 of SEQ ID NO: 1270; 335-359, 461-489, 485-513, and/or 509-536 of SEQ ID NO: 1271; 30-62, and/or 58-90 of SEQ ID NO: 1272; 581-605 of SEQ ID NO: 1273; 101-131, and/or 127-157 of SEQ ID NO: 1274; 317-349, and/or 345-377 of SEQ ID NO: 1275; 534-558 of SEQ ID NO: 1276; 108-133, 234-261, 257-284, 280-307, and/or 303-330 of SEQ ID NO: 1277; 2-27, 23-48, 44-68, 82-106, 104-128, 152-176, and/or 373-397 of SEQ ID NO: 1278; 324-348 of SEQ ID NO: 1279; 196-223, and/or 219-246 of SEQ ID NO: 1280; 263-294, and/or 291-323 of SEQ ID NO: 1281; 183-207 of SEQ ID NO: 1282; 176-200 of SEQ ID NO: 1283; 297-321 of SEQ ID NO: 1284; 91-115 of SEQ ID NO: 1285; 11-41, 37-67, and/or 63-92 of SEQ ID NO: 1286; 157-190, and/or 186-219 of SEQ ID NO: 1287; 240-264 of SEQ ID NO: 1288; 51-75 of SEQ ID NO: 1289; 31-55 of SEQ ID NO: 1290; 166-190 of SEQ ID NO: 1291; 10-34 of SEQ ID NO: 1293; 115-137 of SEQ ID NO: 313; 554-579, 575-600, 596-621, and/or 617-642 of SEQ ID NO: 1292; 200-226, 222-248, and/or 244-269 of SEQ ID NO: 1295; 4-28 of SEQ ID NO: 1296; 476-503 of SEQ ID NO: 1297; 370-394 of SEQ ID NO: 1298; 129-153 of SEQ ID NO: 1299; 51-75 of SEQ ID NO: 1301; 88-112 of SEQ ID NO: 1302; 670-694, and/or 848-872 of SEQ ID NO: 1303; 164-196, 703-731, 764-796, 792-823, 192-223, 219-250, 438-463, 459-483, 479-503, 629-658, 654-683, and/or 679-707 of SEQ ID NO: 1306; 5-36 of SEQ ID NO: 461; 32-51 of SEQ ID NO: 463; 16-46 of SEQ ID NO: 468; 12-33, and/or 27-48 of SEQ ID NO: 469; 10-39 of SEQ ID NO: 470; 27-56, 52-81, 77-105, and/or 101-129 of SEQ ID NO: 471; 30-57, 53-80, and/or 76-102 of SEQ ID NO: 472; 48-77 of SEQ ID NO: 473; 6-35 of SEQ ID NO: 474; 233-257, 253-277, 273-297, and/or; 293-317 of SEQ ID NO: 301; 290-317, and/or; 367-388 of SEQ ID NO: 303; 240-265 of SEQ ID NO: 305; 796-824 of SEQ ID NO: 306; 624-653 of SEQ ID NO: 307; 36-64, 60-87, 83-110, and/or; 106-133 of SEQ ID NO: 308; 120-148 of SEQ ID NO: 309; 71-106 of SEQ ID NO: 310; 741-764, and/or; 760-783 of SEQ ID NO: 311; 450-483 of SEQ ID NO: 314; 111-137 of SEQ ID NO: 316; 896-923 of SEQ ID NO: 317; 565-595 of SEQ ID NO: 319; 178-204, 200-226, 222-248, 244-269, and/or 265-296 of SEQ ID NO: 320; 1343-1369, 180-209, 205-233, and/or; 229-258 of SEQ ID NO: 321; 242-270, 266-294, and/or; 290-318 of SEQ ID NO: 322; 259-268 of SEQ ID NO: 360; 278-305 of SEQ ID NO: 312; 115-137 of SEQ ID NO: 313;89-116, 112-139, and/or; 135-162 of SEQ ID NO: 326; 76-97, and/or; 93-114 of SEQ ID NO: 329; 39-66, 62-88, and/or; 84-110 of SEQ ID NO: 331; 244-274 of SEQ ID NO: 333; 548-572 of SEQ ID NO: 335; 165-200 of SEQ ID NO: 336; 1-35 of SEQ ID NO: 338; 662-695 of SEQ ID NO: 339; 232-256, and/or; 252-283 of SEQ ID NO: 342; 588-620 of SEQ ID NO: 343; 197-229, 225-256, and/or; 252-283 of SEQ ID NO: 345; 212-244, 240-272, and/or; 395-429 of SEQ ID NO: 347; 209-230 of SEQ ID NO: 348; 78-106, and/or 102-130 of SEQ ID NO: 349; 118-142 of SEQ ID NO: 350; 105-133, 129-156, and/or 152-180 of SEQ ID NO: 351; 147-175 of SEQ ID NO: 352; 16-44 of SEQ ID NO: 353; 102-131 of SEQ ID NO: 354; 328-355 of SEQ ID NO: 355; 436-465 of SEQ ID NO: 357; 139-166 of SEQ ID NO: 363; 373-401 of SEQ ID NO: 365; 114-148 of SEQ ID NO: 367; 23-48, 44-70, and/or 71-99 of SEQ ID NO: 369; 34-53 of SEQ ID NO: 425; 164-193, 189-218, and/or 214-244 of SEQ ID NO: 456; 1522-1552 of SEQ ID NO: 431; 17-49 of SEQ ID NO: 475; 122-149 of SEQ ID NO: 459; 106-136 of SEQ ID NO: 460; 26-51, 47-72, and/or 68-92 of SEQ ID NO: 476; 187-208, and/or 204-224 of SEQ ID NO: 477; 654-682 of SEQ ID NO: 361; 944-970, 966-992, and/or 988-1015 of SEQ ID NO: 364; 242-256, and/or 60-88 of SEQ ID NO: 457; 96-124 of SEQ ID NO: 1042; 550-575, 571-596, and/or 592-616 of SEQ ID NO: 1031; 9-22 of SEQ ID NO: 1032; 139-163, 159-183, 179-203, 199-220, 549-576, 572-599, and/or 595-622 of SEQ ID NO: 1007; 70-88 of SEQ ID NO: 302; 134-147 of SEQ ID NO: 303; 378-400 of SEQ ID NO: 315; 3-19 of SEQ ID NO: 318; 103-119 of SEQ ID NO: 323; 266-292 of SEQ ID NO: 324; 179-193 of SEQ ID NO: 325; 282-296 of SEQ ID NO: 327; 718-740 of SEQ ID NO: 328; 1-29, 30-58, and/or 59-87 of SEQ ID NO: 330; 54-69 of SEQ ID NO: 332; 33-56 of SEQ ID NO: 334; 71-84 of SEQ ID NO: 340; 60-70 of SEQ ID NO: 341; 17-41 of SEQ ID NO: 344; 110-127 of SEQ ID NO: 346; 101-122 of SEQ ID NO: 356; 408-427 of SEQ ID NO: 358; 42-61 of SEQ ID NO: 359; 40-59 of SEQ ID NO: 362; 178-193 of SEQ ID NO: 366; 518-545, and/or 541-568 of SEQ ID NO: 368; 564-591 of SEQ ID NO: 368; 415-440, 436-461, 457-482, and/or 478-503 of SEQ ID NO: 462; 1-29, 53-80, 76-103, and/or 99-126 of SEQ ID NO: 464; 342-366, 362-386, 382-406, and/or 402-428 of SEQ ID NO: 465; 342-369, 365-392, 388-415, 411-438, 434-461, and/or 457-484 of SEQ ID NO: 466; 530-558, 554-582, 578-606, and/or 632-659 of SEQ ID NO: 467; 34-42, 56-87, 95-133, 136-146, 157-213, 219-235, 246-282, 313-333, 358-394, and/or 196-215 of SEQ ID NO: 1365; 67-74, 88-94, 112-118, 127-138, 155-169, 171-180, 183-190, 196-205, 243-249, 260-271, 308-344, 346-373, 381-414, 416-457, 473-513, 515-524, 528-535, 539-544, 556-566, 572-580, 585-590, and/or 27-129 of SEQ ID NO: 1366; 4-16, 21-36, 38-47, 54-64, 92-103, 117-126, 135-155, 157-200, 202-223, 231-239, 246-262, and/or 128-153 of SEQ ID NO: 1367; 4-10, 16-30, and/or 11-43 of SEQ ID NO: 1369; 7-22, 52-77, 83-93, 101-111, 125-136, 139-157, 212-221, 231-239, 241-247, 264-273, 275-294, 329-336, 349-357, 365-379, 389-405, 419-434, 439-445, 456-462, 467-481, 493-506, 508-516, 522-545, 547-556, 566-583, 611-627, 655-670, 678-693, 717-724, 734-748, 756-766, 772-790, 797-808, 815-820, 825-831, 833-838, 851-868, 891-917, 919-926, 933-940, 944-953, and/or 246-271 of SEQ ID NO: 1370; 4-12, 14-31, 42-59, 61-69, 73-83, 96-103, 140-149, 153-165, 180-187, 199-208, 222-230, 232-240, 248-254, 256-270, 274-283, 289-299, 302-317, 322-328, 332-345, 351-365, 387-396, 419-432, 441-447, 453-466, 487-505, 508-524, 560-571, 580-590, 592-605, 625-633, 639-647, 652-658, 671-679, 722-728, and/or 660-694 of SEQ ID NO: 1371; 4-13, 26-39, 53-59, 68-74, 88-95, 102-119, 125-132, 136-150, 153-162, 165-175, 188-228, 238-245, 268-283, 285-307, 317-324, 326-343, 350-359, and/or 79-101 of SEQ ID NO: 1372; 10-37, 55-68, 71-78, 92-98, 115-122, 131-138, 149-158, 163-170, 172-189, 212-219, 239-257, 259-271, 289-302, 304-320, 322-340, 359-366, 373-384, 400-412, 444-453, 460-474, 485-527, and/or 187-224 of SEQ ID NO: 1373; 13-21, 27-36, 41-50, 55-64, 66-72, 74-90, 103-112, 127-136, 153-164, 166-186, 193-219, 224-242, 260-273, 278-294, 298-306, 328-334, and/or 142-171 of SEQ ID NO: 1374; 16-29, 31-41, 49-61, 63-81, 86-92, 107-114, 122-135, 155-177, 195-211, 245-252, 264-270, 273-279, 297-322, 327-334, 339-348, 371-378, 380-389, 402-408, 414-421, 424-430, 452-459, 481-488, 500-506, 543-556, 567-573, 588-594, 608-615, 628-633, 668-675, 683-689, 700-706, 735-750, 793-802, 816-822, 841-846, 848-855, 858-864, 894-913, 921-929, 941-948, 974-990, 993-1005, 1053-1068, 1096-1110, 1117-1123, 1126-1145, 1149-1168, 1191-1203, 1219-1225, 1239-1248, 1253-1265, 1297-1309, 1356-1370, 1373-1379, 1388-1395, and/or 372-393 of SEQ ID NO: 1375; 5-15, 29-37, 39-46, 51-60, 65-71, 73-97, 105-131, 137-152, 154-161, 173-185, 193-210, 214-222, 224-232, 241-255, 266-274, 277-289, 291-303, 307-338, 345-352, 358-371, 389-395, 402-422, 433-440, 444-465, 471-478, 498-513, 524-536, 542-558, 561-576, 584-602, 604-623, 631-639, 643-658, 667-683, 689-716, and/or 315-397 of SEQ ID NO: 1376; 7-25, 30-37, 39-60, 69-86, and/or 75-89 of SEQ ID NO: 1377; 16-30 of SEQ ID NO: 1378; 8-27 of SEQ ID NO: 1379; 4-22, 29-48, 50-58, 62-69, 71-78, and/or 6-36 of SEQ ID NO: 1380; 4-23, and/or 3-24 of SEQ ID NO: 1381; 4-12, 30-66, and/or 11-33 of SEQ ID NO: 1382; 4-9, 40-64, 68-82, and/or 54-72 of SEQ ID NO: 1383; 4-42, and/or 11-39 of SEQ ID NO: 1384; 15-24, 77-88, 90-103, 116-123, 134-144, and/or 23-33 of SEQ ID NO: 1385; 17-25, 47-56, 68-79, and/or 64-74 of SEQ ID NO: 1386; 4-12, 14-21, 27-33, and/or 7-25 of SEQ ID NO: 1387; 4-10, 25-37, and/or 5-23 of SEQ ID NO: 1388; 4-9, 14-29, 38-44, 49-67, 69-97, 118-128, 135-146, 151-157, 159-172, 196-203, 227-241, 253-259, 262-271, 284-295, 299-309, 343-353, 356-362, 392-406, 410-416, 429-441, 463-478, 503-509, and/or 495-511 of SEQ ID NO: 1389; 4-12, 24-33, 39-51, 57-63, 78-87, and/or 32-56 of SEQ ID NO: 1390; 8-15, 29-40, 48-54, and/or 33-56 of SEQ ID NO: 1391; 12-61, and/or 35-61 of SEQ ID NO: 1392; 8-27 of SEQ ID NO: 1393.

41. The hyperimmune serum-reactive antigen or fragment of claim 38, comprising at least 6 contiguous amino acids of any of SEQ ID NOs: 302-336, 338-600, 763-924, 997-1068, 1203-1336, or 1365-1393.

42. The hyperimmune serum-reactive antigen or fragment of claim 38, comprising at least 8 contiguous amino acids of any of SEQ ID NOs: 302-336, 338-600, 763-924, 997-1068, 1203-1336, or 1365-1393.

43. The hyperimmune serum-reactive antigen or fragment of claim 38, comprising at least 10 contiguous amino acids of any of SEQ ID NOs: 302-336, 338-600, 763-924, 997-1068, 1203-1336, or 1365-1393.

44. The hyperimmune serum-reactive antigen or fragment of claim 38, further defined as directed against an enteroaggregative E. coli, enterotoxigenic E. coli, S. flexneri and/or C. jejuni.

45. A pharmaceutical composition comprising a hyperimmune serum-reactive antigen or fragment of claim 38.

46. The pharmaceutical composition of claim 45, wherein the hyperimmune serum-reactive antigen or fragment is directed against an enteroaggregative E. coli, enterotoxigenic E. coli, S. flexneri and/or C. jejuni.

47. The pharmaceutical composition of claim 45, further defined as comprising at least two different hyperimmune serum-reactive antigens and/or fragments.

48. The pharmaceutical composition of claim 47, wherein the at least two different hyperimmune serum-reactive antigens and/or fragments are both directed against an enteroaggregative E. coli, enterotoxigenic E. coli, S. flexneri and/or C. jejuni.

49. The pharmaceutical composition of claim 45, further comprising an immunostimulatory substance.

50. The pharmaceutical composition of claim 49, wherein the immunostimulatory substance is a polycationic polymer, an immunostimulatory deoxynucleotide (ODN), a peptide containing at least two LysLeuLys motifs, a neuroactive compound, alum, or a Freund's complete or incomplete adjuvant.

51. The pharmaceutical composition of claim 50, wherein the polycationic polymer is a polycationic peptide.

52. The pharmaceutical composition of claim 50, wherein the neuroactive compound is human growth hormone.

53. The pharmaceutical composition of claim 45, further defined as a vaccine.

54. The pharmaceutical composition of claim 53, further defined as a vaccine for treatment and/or prevention of an enteroaggregative E. coli, enterotoxigenic E. coli, S. flexneri and/or C. jejuni infection.

55. A method of vaccinating a subject comprising:
obtaining a pharmaceutical composition of claim 45; and
administering the pharmaceutical composition to a subject; wherein the subject is vaccinated.

56. The method of claim 55, wherein the subject is a human.

57. The method of claim 55, further defined as a method of treating and/or preventing an enteroaggregative E. coli, enterotoxigenic E. coli, S. flexneri and/or C. jejuni infection in the subject.

58. The method of claim 55, wherein the hyperimmune serum-reactive antigen or fragment is directed against an enteroaggregative E. coli, enterotoxigenic E. coli, S. flexneri and/or C. jejuni.
